(19) United States
(12) Patent Application Publication
(10) Pub. No.: US 2025/0215456 A1
(43) Pub. Date: Jul. 3, 2025

(54) POXVIRUS-BASED VECTORS PRODUCED BY NATURAL OR SYNTHETIC DNA AND USES THEREOF

(71) Applicant: CITY OF HOPE, Duarte, CA (US)

(72) Inventors: Don J. DIAMOND, Glendora, CA (US); Felix WUSSOW, Glendora, CA (US)

(73) Assignee: CITY OF HOPE, Duarte, CA (US)

(21) Appl. No.: 19/076,904

(22) Filed: Mar. 11, 2025

Related U.S. Application Data
(63) Continuation of application No. 17/759,254, filed on Jul. 19, 2023.
(60) Provisional application No. 63/112,803, filed on Nov. 12, 2020.
(60) Provisional application No. 62/969,628, filed on Feb. 3, 2020.

(51) Int. Cl. C12N 15/86; A61K 39/00; A61K 39/215; A61K 39/245; A61P 31/14; A61P 31/22; A61P 35/00

(52) U.S. Cl. CPC C12N 15/86; A61K 39/001151; A61K 39/215; A61K 39/245; A61P 31/14; A61P 31/22; A61P 35/00; A61K 2039/5256; C12N 2710/16134; C12N 2710/16171; C12N 2710/24134; C12N 2710/24143; C12N 2710/24152; C12N 2710/24171; C12N 2770/20034; C12N 2770/20071

(57) ABSTRACT

Disclosed are methods of producing poxvirus-based vectors or recombinant poxvirus-based vectors from naturally derived, chemically synthesized DNA fragments, or a combination of naturally derived and chemically synthesized DNA fragments. One or more DNA sequences encoding one or more antigens, subunits or fragments thereof or other heterologous gene sequences are inserted in one or more poxvirus insertion sites in one or more DNA fragments. The methods include transfecting a host cell with one or more circular or linear DNA fragments such that a poxvirus or recombinant poxvirus is reconstituted in the host cell, the reconstituted poxvirus or recombinant poxvirus comprising the genome of a desired poxvirus. Also disclosed are poxviruses or recombinant poxviruses produced by the technology and uses thereof.

### CROSS-REFERENCE TO RELATED APPLICATION(S)

This application is a continuation of U.S. patent application Ser. No. 17/759,254, filed Jul. 21, 2022, which is a United State National Phase Application of International Patent Application No. PCT/US2021/016247, filed Feb. 2, 2021, which claims priority to U.S. Provisional Patent Application No. 62/969,628, filed Feb. 3, 2020, and U.S. Provisional Patent Application No. 63/113,803, filed Nov. 13, 2020, the contents of which are hereby incorporated by reference in their entirety.

### SEQUENCE LISTING

This application contains a ST.26 compliant Sequence Listing, which was submitted in XML format via Patent Center, and is hereby incorporated by reference in its entirety. The XML copy, created on Mar. 10, 2025, is named 0544358200US03.xml and is 526,000 bytes in size.

### BACKGROUND

Poxviruses are large double-stranded, enveloped DNA viruses that replicate entirely in the cytoplasm of infected cells by encoding their own enzymes for DNA transcription and replication^{20,27}. One of the greatest achievements in medical history was the eradication of the causative agent of smallpox (Variola virus) by mass vaccination with replication-competent, attenuated Vaccinia virus vaccine strains. Vaccinia virus is used as the prototype member to study poxvirus replication and has been further developed to generate recombinant vaccines and oncolytic agents.

Modified Vaccinia Ankara (MVA) is a highly attenuated orthopoxvirus that was derived from its parental strain Chorioallantois Vaccinia Ankara (CVA) by 570 passages on chicken embryo fibroblasts (CEF)¹. As a result of the attenuation process MVA has acquired six major genome deletions (Del1-6) as well as multiple shorter deletions, insertions, and point mutations, leading to gene fragmentation, truncation, short internal deletions, and amino acid substitutions². While all of these mutations are likely to contribute to the highly-attenuated phenotype of MVA, the precise genetic determinants that are associated with the MVA assembly deficiency remain obscure^{2,3}. MVA has severely restricted host cell tropism, allowing productive assembly only in avian cells, e.g. CEF and baby hamster kidney (BHK) cells, whereas in human and most other mammalian cells, MVA assembly is abortive due to a late block in virus assembly^{4,5}. Although non-pathogenic and highly attenuated, MVA maintains excellent immunogenicity as demonstrated in various animal models and humans^{6,7}. In the late phase of the smallpox eradication campaign, MVA was used as a priming vector for the replication competent vaccinia-based vaccine in over 120,000 individuals in Germany and no adverse events were reported⁸. In the past decades, MVA has been developed as a stand-alone smallpox vaccine and is currently pursued by the United States (US) government as a safer alternative to substitute the existing vaccinia-based vaccine stocks as a preventative countermeasure in case of a smallpox outbreak⁹⁻¹¹. The FDA approved MVA, under the trade name Jynneos (Bavarian Nordic) on Sep. 24, 2019 to prevent both smallpox and monkey pox. Previously, the identical MVA vaccine using the trade name Imvamune was approved in Europe as a smallpox vaccine.

All currently used MVA vectors or derivatives thereof are licensed or owned by academic, commercial, or governmental entities, which greatly restricts their use to develop MVA-based vaccine vectors. Therefore, there is a need to develop alternative MVA vectors for various research, prophylactic, and therapeutic uses. In addition, novel technologies are urgently needed to accelerate the development of recombinant poxvirus vectors for pathogen preparedness and disease prevention.

### SUMMARY

In one aspect, this disclosure relates to a method of producing a poxvirus vector or a recombinant poxvirus vector. The method entails the steps of transfecting one or more DNA fragments into a host cell, wherein the one or more DNA fragments comprise the entire genomic DNA sequence of a desired poxvirus, such that the poxvirus is reconstituted in the host cell. In certain embodiments, two or more DNA fragments are co-transfected into the host cell, each DNA fragment comprises a partial sequence of the poxvirus genome such that the two or more DNA fragments are assembled sequentially by homologous recombination and comprise the full-length sequence of the poxvirus genome when reconstituted in the host cell. In certain embodiments, the method further entails infecting the host cell with a helper virus before, during, or after the transfection of the one or more DNA fragments to initiate the transcription of the one or more DNA fragments. In certain embodiments, the helper virus is Fowl pox virus (FPV), sheep fibroma virus, vaccinia virus, or cowpox virus. In certain embodiments, the one or more DNA fragments are circularized before transfection or transfected in circular forms into the host cell. In certain embodiments, the one or more DNA fragments are cloned into a plasmid or a bacterial artificial chromosome (BAC) vector. In certain embodiments, the one or more DNA fragments are linearized before co-transfection or transfected in linearized forms into the host cell. In certain embodiments, the one or more DNA fragments are naturally derived, chemically synthesized, or a combination of naturally derived and chemically synthesized DNA fragments. In certain embodiments, the poxvirus genomic sequence comprises the sequence of Modified Vaccinia Ankara (MVA) Accession No. #U94848 or #AY603355. In certain embodiments, the poxvirus genomic sequence comprises the sequence of Vaccinia virus genome. In certain embodiments, two adjacent DNA fragments have an overlapping sequence to facilitate homologous recombination. In certain embodiments, the overlapping sequence is between about 100 bp and about 5000 bp in length. In certain embodiments, the one or more DNA fragments further comprise an inverted terminal repeat (ITR) region. In certain embodiments, the one or more DNA fragments further comprise a poxvirus terminal hairpin loop (HL) sequence, a poxvirus genome resolution (CR) sequence, or both, wherein the HL or the CR sequence is added to one or both ends of the DNA fragment as single stranded or double stranded DNA sequences in sense or antisense orientation. In certain embodiments, the one or more DNA fragments further comprise one or more HL sequences and one or more CR sequences. In certain embodiments, each HL sequence is flanked by two CR sequences at both ends of the HL sequence. In certain embodiments, wherein only a subset of the one or more DNA fragments comprise the HL or CR sequence. In certain embodiments, the one or more DNA fragments further comprise one or more DNA sequences encoding one or more antigens, subunits, or fragments thereof or other heterologous DNA sequences. In certain embodiments, two or more DNA fragments comprise the DNA sequence of the same antigen, a subunit or fragment thereof or the same heterologous DNA sequence. In certain embodiments, two or more DNA fragments comprise the DNA sequences of different antigens, subunits, or fragments thereof or other heterologous DNA sequences. In certain embodiments, the DNA sequences of the antigens, subunits, or fragments thereof or other heterologous DNA sequences are codon optimized for expression in the host cell. In certain embodiments, the one or more DNA fragments further comprise a virus promoter upstream of the DNA sequences of the antigens, subunits, or fragments thereof or other heterologous DNA sequences, a transcription termination signal downstream the DNA sequences of the antigens, subunits, or fragments thereof or other heterologous DNA sequences, or both. In certain embodiments, the DNA sequences encoding the antigens, subunits, or fragments thereof or other heterologous DNA sequences are inserted in one or more poxvirus insertion sites such as intergenic regions, non-essential genes and regions, and deletion sites.

In another aspect, disclosed herein is an expression system comprising: (i) a single DNA fragment comprising the entire genome of a desired poxvirus, or two or more DNA fragments each comprising a partial sequence of the genome of the desired poxvirus such that the two or more DNA fragments, when transferred into the host cell upon co-transfection, are assembled sequentially and comprise the full-length sequence of the poxvirus genome and enable reconstitution of the poxvirus, and (ii) one or more DNA sequences encoding one or more antigens, subunits, or fragments thereof or other heterologous DNA sequences inserted in one or more insertion sites of the poxvirus, wherein the antigens or subunits thereof or other heterologous DNA sequences are expressed in the host cell upon transfection of the one or more poxvirus DNA fragments and reconstitution of the poxvirus. In certain embodiments, the one or more DNA fragments are circularized before transfection or transfected in circular forms into the host cell. In certain embodiments, the one or more DNA fragments are cloned into a plasmid or a BAC vector. In certain embodiments, the one or more DNA fragments are linearized before transfection or transfected in linearized forms into the host cell. In certain embodiments, the one or more DNA fragments are naturally derived, chemically synthesized, or a combination of naturally derived and chemically synthesized DNA fragments. In certain embodiments, the genomic sequence of the poxvirus comprises the sequence of MVA Accession No. #U94848 or #AY603355. In certain embodiments, the two adjacent DNA fragments have an overlapping sequence to facilitate homologous recombination. In certain embodiments, the overlapping sequence is between about 100 bp and about 5000 bp in length. In certain embodiments, the one or more DNA fragments further comprise an inverted terminal repeat (ITR) region. In certain embodiments, the one or more DNA fragments further comprise a poxvirus terminal hairpin loop (HL) sequence, a poxvirus genome resolution (CR) sequence, or both, wherein the HL or the CR sequence is added to one or both ends of the DNA fragment as single stranded or double stranded DNA sequences in sense or antisense orientation. In certain embodiments, the one or more DNA fragments further comprise one or more HL sequences and one or more CR sequences. In certain embodiments, each HL sequence is flanked by two CR sequences at both ends of the HL sequence. In certain embodiments, only a subset of the one or more DNA fragments comprises the HL or CR sequence. In certain embodiments, the one or more DNA fragments further comprise a virus promoter upstream of the DNA sequences encoding the antigens, subunits or fragments thereof or other heterologous DNA sequences, a transcription termination signal downstream of the DNA sequences encoding the antigens, subunits or fragments thereof or other heterologous DNA sequences, or both. In certain embodiments, the DNA sequences encoding one or more antigens, subunits or fragments thereof or other heterologous DNA sequences are inserted in one or more poxvirus insertion sites.

In another aspect, disclosed herein is a vaccine composition for preventing or treating cancer or an infectious disease comprising: (i) a single DNA fragment comprising the entire genome of a desired poxvirus, or two or more DNA fragments each comprising a partial sequence of the genome of the desired poxvirus such that the two or more DNA fragments, when transferred into the host cell upon co-transfection, are assembled sequentially and comprise the full-length sequence of the poxvirus genome and enable reconstitution of the poxvirus, and (ii) one or more DNA sequences encoding one or more antigens, subunits, or fragments thereof or other heterologous DNA sequences inserted in one or more insertion sites of the poxvirus, wherein the antigens, subunits or fragments thereof or other heterologous DNA sequences are expressed in the host cell upon transfection of the one or more DNA fragments and reconstitution of the poxvirus. In certain embodiments, the antigens, subunits or fragments thereof or other heterologous DNA sequences are inserted in one or more poxvirus insertion sites. In certain embodiments, the vaccine composition further comprises a pharmaceutically acceptable carrier, adjuvant, additive or combination thereof.

In yet another aspect, disclosed herein is a method of preventing or treating cancer or a viral infection in a subject comprising administering a prophylactically or therapeutically effective amount of a vaccine composition to the subject, wherein the vaccine comprises: (i) a single DNA fragment comprising the entire genome of a desired poxvirus, or two or more DNA fragments each comprising a partial sequence of the genome of the desired poxvirus such that the two or more DNA fragments, when transferred into the host cell upon co-transfection, are assembled sequentially and comprise the full-length sequence of the poxvirus genome and enable reconstitution of the poxvirus, and (ii) one or more DNA sequences encoding one or more antigens, subunits or fragments thereof or other heterologous DNA sequences inserted in one or more insertion sites of the poxvirus, wherein the antigens, subunits, or fragments thereof or other heterologous DNA sequences are expressed in the host cell upon transfection of the one or more DNA fragments and reconstitution of the poxvirus. In certain embodiments, the antigens, subunits, or fragments thereof or other heterologous DNA sequences are inserted in one or more poxvirus insertion sites.

### BRIEF DESCRIPTION OF THE DRAWINGS

FIGS. 1A-1C show the design of the sMVA construction. FIG. 1A: Schematic of MVA genome. The MVA genome is approximately 178 kbp in length and contains an internal unique region (UR) flanked by large ˜9.6 kbp long inverted terminal repeats (ITRs). FIG. 1B: Schematic of sMVA fragments. Each of the three sMVA fragments is approximately 60 kbp in length. sMVA fragment 1 (F1) contains the sequence of the left part of the MVA genome, including the left ITR; sMVA fragment 2 (F2) contains the sequence of the middle part of the MVA genome; and sMVA fragment 3 (F3) contains the sequence of the right part of the MVA genome, including the right ITR. sMVA F1 and F2 as well as sMVA F2 and F3 share ˜3 kbp overlapping sequences for homologous recombination (dotted crossed lines). Indicated are the approximate positions of commonly used MVA insertion sites, including Del2, IGR44/45 (44/45), and IGR64/65 (69/70) within sMVA F1, IGR69/70 (69/70) and the TK insertion site within sMVA F2, and Del3 within sMVA F3. FIG. 1C: Schematic of the terminal HL and CR sequences. Each of the sMVA fragments contains at both ends a sequence composition comprising a duplex copy of the MVA terminal HL and flanking CR sequences to promote genome resolution and packaging. Sfil and Fsel restriction sites are included as indicated at both ends of each of the sMVA fragments to release the fragments from the bacterial vector backbone (pCCI-Brick) with or without the terminal CR/HL/CR sequences by enzymatic digestion.

FIG. 2 shows reconstitution of sMVA. A schematic for the generation of sMVA (sMVA) using the three sMVA fragments (F1-F3) as shown in FIG. 1 is depicted. The three sMVA fragments are stably maintained in E. coli, then isolated from the bacteria and transferred by transfection as circular or linearized DNA molecules into MVA permissive BHK or CEF cells. The transfected cells are subsequently infected with Fowl pox virus (FPV) as a helper virus to initiate MVA genome transcription and replication and consequently the reconstitution of the viral form of sMVA.

FIG. 3 depicts the construction of recombinant sMVA with one antigen. A schematic is shown for an example of the construction of recombinant sMVA (rsMVA) with one introduced antigen sequence, heterologous gene sequence, or other genome alteration. The antigen or heterologous gene sequence (black circle) is inserted into the MVA genome sequence of F2 (or one of the other sMVA fragments (F1 or F3)) by bacterial recombination methods in E. coli using linear PCR-derived products or other forms of linear DNA constructs. The modified sMVA fragment F1 and the unmodified sMVA fragments F2 and F3 are isolated from E. coli and co-transfected as circular or linearized forms into FPV-infected BHK or CEF cells to initiate the reconstitution of rsMVA expressing one antigen or heterologous gene sequence. Nucl.=cell nucleus.

FIG. 4 illustrates the construction of recombinant sMVA with three antigens. A schematic is shown for an example of the generation of a recombinant sMVA (rsMVA) with 3 introduced antigen sequences, heterologous gene sequences, or other genome alterations. An antigen or heterologous gene sequence (shown in square, circle, or rectangle) is inserted into each of the three sMVA fragments (F1-F3) by bacterial recombination techniques in E. coli using linear PCR-derived products or other forms of linear DNA constructs. The modified F1, F2, and F3 sMVA fragments are isolated from E. coli and co-transfected in circular or linearized forms into permissive BHK or CEF cells, where rsMVA virus expressing three antigen or heterologous gene sequences are reconstituted in the presence of FPV as a helper virus. Nucl.=cell nucleus.

FIG. 5 illustrates the sMVA reconstitution process. A schematic is shown for an example of the tissue culture reconstitution process of sMVA or rsMVA using the three sMVA fragments (F1-F3) or modified forms thereof. BHK cells seeded in 6-well tissue culture format are co-transfected with the three sMVA fragments (unmodified, modified, or a combination thereof) and subsequently infected with FPV as a helper virus to initiate the transcription and replication of the sMVA fragments and, consequently, the reconstitution of sMVA virus. The transfected/infected BHK cells are transferred every other day (at 2, 4, and 6 days post transfection/infection (dpt/i)) into larger tissue culture formats as indicated to promote sMVA reconstitution. Plaque development and progression can be detected after the day 2 and day 4 cell transfers, resulting in 90-100% infected BHK cells at 7 or 8 dpt/i after the day 6 transfer.

FIGS. 6A-6B show sMVA infectivity analysis. FIG. 6A is a plaque analysis showing representative bright field microscopy images of 5 individual plaques in BHK cell monolayers infected with sMVA or MVA NIH clone 1 at 1 day post infection (dpi). FIG. 6B shows the progression of infection. Shown are representative bright field microscopy images of BHK cell monolayers infected with sMVA or MVA NIH clone 1 at 1, 2, and 3 dpi. Mock-infected (uninfected) BHK cell monolayers were analyzed as control. Plaques and infected areas were visualized by immunostaining for the Vaccinia virus B5R protein.

FIGS. 7A-7L show PCR analysis of the reconstituted sMVA. BHK cells were infected at MOI 5 with sMVA virus reconstituted from the three sMVA fragments (F1-F3) or MVA NIH clone 1 and DNA extracted from infected BHK cells was evaluated by PCR. DNA extracted from mock-infected (uninfected) BHK cells (BHK) as well as H₂O only (Ctrl) was analyzed for control. The PCR analysis included sequence detection of the inverted terminal repeats (ITR) (FIG. 7A); the transition from the left ITR into the internal unique region (LITR/UR) (FIG. 7B) and the MVA Deletion 2 site (Del2) (FIG. 7C) located in sMVA F1-derived DNA; the intergenic region (IGR) between MVA 69R and 70L (IGR69/70) (FIG. 7D) located in sMVA F2-derived DNA; the MVA Deletion 3 site (Del3) (FIG. 7E) and the transition from the internal UR into the right ITR (UR/RITR) (FIG. 7F) located in sMVA F3-derived DNA; the reconstitution of the recombination site of sMVA F1 and F2 (F1/F2) (FIG. 7G) as well as of F2 and F3 (F2/F3) (FIG. 7H); and the MVA genome positions containing the five nucleotide polymorphism (Polym. 1, 2, 3, 4/5) (FIGS. 7I-7L) specific for MVA strain Antoine. The expected PCR products are indicated by the arrows at the right side of each panel. The expected sizes of the PCR products are given in the brackets below each panel.

FIG. 8 shows reconstitution of sMVA with a single fluorescence marker. Shown are representative immunofluorescence and bright field microscopy images of BHK cell monolayers at 6 and 7 days post transfection/infection (dpt/i) using unmodified sMVA fragments F1 and F3 and modified sMVA fragment F2 with inserted red fluorescence protein (RFP) marker (F2-RFP). The sMVA reconstitution procedure from these fragments is illustrated in FIG. 5 and FPV was used as a helper virus. Mock-transfected/infected BHK cell monolayers were analyzed as control.

FIG. 9 shows reconstitution of sMVA with double fluorescence markers. Shown are representative immunofluorescence and bright field microscopy images of BHK cell monolayers at 6 and 7 days post transfection/infection (dpt/i) using unmodified sMVA fragment F1, modified sMVA fragment F2 with inserted red fluorescence protein (RFP) marker (F2-RFP), and modified sMVA fragment F3 with inserted blue fluorescence protein (BFP) marker (F3-BFP). The sMVA reconstitution procedure from these fragments is illustrated in FIG. 5 and FPV was used as a helper virus. Mock-transfected/infected BHK cell monolayers were analyzed as control.

FIGS. 10A-10H illustrate various examples of rsMVA HCMV vector construction. Shown are examples for the use of the sMVA fragments F1-F3 to generate rsMVA vectors with inserted HCMV antigen sequences based on the five subunits of the pentamer complex (PC; including UL128, UL130, UL131A, gH, and gL), glycoprotein B (gB), phosphoprotein 65 (pp65), and/or the immediately-early proteins 1 and 2 (IE1 and IE2). The HCMV antigen sequences are inserted as indicated either separately or combined as 2A-linked polycistronic expression constructs into different MVA insertion sites, including the MVA Deletion 3 (Del3) site or intergenic regions between open reading frames 044/045 (44/45), 064/065 (64/65), or 069/070 (69/70). Examples are provided for rsMVA vectors expressing all PC subunits (sMVA-PC1 (FIG. 10A) and sMVA-PC2 (FIG. 10B)), all PC subunits together with gB and pp65 (sMVA-7Ag1 (FIG. 10C) and sMVA-7Ag2 (FIG. 10D)), or all PC subunits, gB, and pp65 together with IE1/IE2 antigens (sMVA-8Ag1/2 (FIGS. 10E-10F)) and sMVA-9Ag2 (FIGS. 10G-10H)). mH5 represents Vaccinia modified H5 promoter, and ITR represents inverted terminal repeats.

FIGS. 11A-11B show sMVA characterization. FIG. 11A: PCR analysis. CEF infected with sMVA, derived with FPV HP1.441 (sMVA hp) or TROVAC from two independent virus reconstitutions (sMVA tv1 and sMVA tv2), were investigated by PCR for several MVA genome positions (ITR sequences, transition left or right ITR into internal unique region (left ITR/UR; UR/right ITR), Del2, IGR69/70 and Del3 insertion sites, and F1/F2 and F2/F3 recombination sites) and absence of BAC vector sequences. PCR reactions with wtMVA-infected and uninfected cells, without sample (mock), or with MVA BAC were performed as controls. FIG. 11B: Restriction fragment length analysis. Viral DNA isolated from ultra-purified sMVA (sMVA tv1 and sMVA tv2) or wtMVA virus was compared by Kpnl and Xhol restriction enzyme digestion.

FIGS. 12A-12D show sMVA replication properties. The replication properties of sMVA derived with FPV HP1.441 (sMVA hp) or TROVAC from two independent sMVA virus reconstitution (sMVA tv1 and sMVA tv2) were compared with wtMVA. FIG. 12A: Viral foci. CEF infected at low multiplicity of infection (MOI) with the reconstituted sMVA virus or wtMVA were immunostained using anti-Vaccinia polyclonal antibody (aVAC). FIG. 12B: Replication kinetics. BHK or CEF cells were infected at 0.02 MOI with sMVA or wtMVA and viral titers of the inoculum and infected cells at 24 and 48 hours post infection were determined on CEF. Mixed-effects model with the Geisser-Greenhouse correction was applied; at 24 and 48 hours post-infection differences between groups were not significant. FIG. 12C: Viral foci size analysis. BHK or CEF cell monolayers were infected at 0.002 MOI with sMVA or wtMVA and areas of viral foci were determined at 24 hours post infection following immunostaining with aVAC antibody. FIG. 12D: Host cell range analysis. Various human cell lines (HEK293, A549, 143b, and HeLa), CEF or BHK cells were infected at 0.01 MOI with sMVA or wtMVA and virus titers were determined at 48 hours post infection on CEF. Dotted lines indicate the calculated virus titer of the inoculum based on 0.01 MOI. Differences between groups in FIGS. 12C-12D were calculated using one-way ANOVA followed by Tukey's (2C) or Dunnett's (2D) multiple comparison tests. ns=not significant.

FIGS. 13A-13D demonstrate sMVA in vivo immunogenicity. sMVA derived either with FPV HP1.441 (sMVA hp) or TROVAC from two independent virus reconstitution (sMVA tv1 and sMVA tv2) was compared by in vitro analysis with wtMVA. C57BL/6 mice were immunized twice at three-week interval with low (1×10⁷ PFU) or high (5×10⁷ PFU) dose of sMVA or wtMVA. Mock-immunized mice were used as controls. FIG. 13A: Binding antibodies. MVA-specific binding antibodies (IgG titer) stimulated by sMVA or wtMVA were measured after the first and second immunization by ELISA. FIG. 13B: NAb responses. MVA-specific NAb titers induced by sMVA or wtMVA were measured after the booster immunization against recombinant wtMVA expressing a GFP marker. FIGS. 13C-13D: T cell responses. MVA-specific IFNγ, TNFα, IL-4, and IL-10-secreting CD8+ (13C) and CD4+ (13D) T cell responses induced by sMVA or wtMVA after two immunizations were measured by flow cytometry following ex vivo antigen stimulation using B8R immunodominant peptides. Differences between groups were evaluated using one-way ANOVA with Tukey's multiple comparison test. ns=not significant.

FIGS. 14A-14D demonstrate sMVA immunogenicity in vivo. sMVA derived either with FPV strain HP1.441 (sMVA hp) or with FPV strain TROVAC from two independent virus reconstitution (sMVA tv1 and sMVA tv2) was compared by in vitro analysis with wtMVA. C57BL/6 mice (N=4) were immunized twice in a three-week interval with low (1×10⁷ PFU) or high (5×10⁷ PFU) dose of sMVA or wtMVA. Mock-immunized mice were used as controls. FIG. 14A: Binding antibodies. Shown is the absorbance at 450 nm at different serum dilutions of MVA-specific binding antibodies (IgG titer) measured by ELISA after the first and second immunization in mice receiving sMVA or wtMVA. FIG. 14B: NAb responses. MVA-specific NAb titers induced by sMVA or wtMVA were measured after the booster immunization against wtMVA expressing a GFP marker. Shown is the measured GFP area of infected cells in square pixels (pix²×10³) at different serum dilutions. FIGS. 14C-14D: T cell responses. MVA-specific CD8+ (14C) and CD4+ (14D) T cells expressing IFNγ, TNFα, IL-4, and IL-10 were measured after two immunizations with sMVA or wtMVA by flow cytometry following ex vivo antigen stimulation using Vaccinia A19L immunodominant peptides. Differences between groups were evaluated using one-way ANOVA with Tukey's multiple comparison test. ns=not significant.

### DETAILED DESCRIPTION

Disclosed herein are methods of producing poxvirus-based vectors and recombinant poxvirus vectors from circularized or linearized, naturally derived or chemically synthesized DNA. Specific examples are provided herein to produce a fully synthetic version of MVA (sMVA) from circularized synthetic DNA fragments as well as recombinant sMVA (rsMVA) expressing one or more heterologous gene sequences, including fluorescence markers or infectious disease and cancer antigens.

Because of its excellent safety profile in addition to its versatile expression system and large capacity to accommodate foreign DNA sequences (up 30 kbp)¹, MVA is widely used to develop recombinant vaccine vectors against infections disease and cancer^{7,12,13}. MVA has been pursued to develop different vaccine strategies for cancer treatment^{14,15} as well as various vaccine approaches to prevent human cytomegalovirus (HCMV) infection¹⁶⁻¹⁸, a common cause of permanent birth defects in newborns and complications in transplant recipients¹⁹. Some of these vaccines have completed clinical phase I or II evaluation^{14,17}. As a member of the poxvirus family, MVA replicates entirely in the cytoplasm of infected cells, providing its own enzymes for transcription and DNA replication^{1,20}. Although MVA virus production is abortive because of a late block in assembly in mammalian cells, MVA can efficiently infect most mammalian cells, including human cells, and initiate robust gene expression and DNA replication, making MVA an ideal vehicle to efficiently deliver and express foreign antigens in vitro and in vivo^{1,5}. The most commonly used method to generate MVA recombinants is based on the so-called transfection/infection method using a transfer plasmid, which is co-delivered together with MVA into permissive cells (CEF or BHK), thereby inserting a desired antigen together with an upstream promoter sequence and downstream transcription termination signal into the MVA genome by spontaneous homologous recombination^{1,5,21}.

Although this method is widely used, it can be laborious and is hampered by several rounds of selection during antigen insertion and additionally during subsequent marker removal to obtain homogenous populations of recombinant MVA²¹. These drawbacks in the conventional transfection/infection method can be particular problematic for the generation of multiantigenic MVA vectors with antigens inserted into two or more insertion sites, which can reduce MVA vaccine stability^{18,22}. As an alternative to the conventional transfection/infection method and to facilitate the generation of multiantigenic MVA vectors, methods have been developed to generate MVA recombinants using bacterial artificial chromosome (BAC) technology²³⁻²⁵. These methods allow repeated manipulation to insert antigens into the MVA genome by highly efficient and versatile mutagenesis techniques and to reconstitute homogenous virus populations of recombinant MVA in BHK cells with Fowl poxvirus (FPV) as a helper virus, which is required to “jump-start” the transcription of the non-infectious MVA genome²³.

### Construction of Synthetic Poxviruses

Disclosed herein are methods of producing poxvirus-based vectors or recombinant poxvirus vectors from naturally derived or chemically synthesized DNA. In certain embodiments, a single DNA fragment is derived from viral DNA or chemically synthesized and comprises the entire genome sequence of a poxvirus. This single DNA fragment can be used to transfect a host cell such that the poxvirus is reconstituted. In other embodiments, two or more naturally derived or chemically synthesized DNA fragments, or a combination thereof, are used to co-transfect a host cell, wherein each DNA fragment comprises a partial sequence of the poxvirus genomic DNA with overlapping sequences at the ends of two adjacent DNA fragments, such that when the two or more DNA fragments are co-transfected into the host cell, they assemble with each other by homologous recombination to form a poxvirus comprising a full-length sequence of the desired poxvirus genome. In certain embodiments, the overlapping sequence is between about 100 bp and about 5000 bp in length.

In certain embodiments, a shortened genomic sequence rather than the entire genomic sequence or an altered genomic sequence with deletion(s) or modification(s) in non-essential genes or regions of the genomic sequence of a poxvirus, or a hybrid derivative comprising the genomic sequences from two or multiple different poxviruses can be used to produce vectors disclosed herein.

In certain embodiments, one or more naturally derived or chemically synthesized DNA fragment(s) comprising the poxvirus genome or subgenomic DNA may be further modified to form artificial hybrid fragments composed of natural and synthetic poxvirus genomic DNA sequences. In other embodiments, the naturally derived or chemically synthesized one or more DNA fragment(s) maybe composed of sequences derived from two different poxviruses to form poxvirus hybrid sequences. One or more DNA fragment(s) can be composed of poxvirus sequences or sequences composed of different poxvirus sequences derived from MVA (NCBI accession #U94848, #AY603355), Vaccinia virus (#NC_006998, #LT966077), Camelpox virus (#NC_003391), Cowpox virus (#NC_003663) Ectromelia virus (#NC_004105), Monkeypox virus (#NC_003310), Racoonpox virus (#NC_027213), Skunkpox virus (#NC_031038), Taterapox virus (#NC_008291), Variola virus (#NC_001611, #L22579), Velopox virus (#NC_031033), Canarypox virus (#NC_005309), Swinepox virus (#NC_003389), FPV (#NC_002188, #MH734528), Myxoma virus (#GQ409969), Sheeppox virus (NC_004002), Goatpox virus (#NC_004003), (Orf virus #NC_005336), Rabbit fibroma virus (#NC_001266), any strain variations of these poxviruses, or any other poxvirus or strain variations thereof.

In certain embodiments, the host cell is infected with a helper virus such as FPV before, during, or after the transfection of one or more DNA fragments comprising the sequence of the poxvirus genome or subgenomic DNA. A helper virus can be any suitable virus that infects the host cell and allows initiation of the transcription and replication of the poxvirus. Used herein as an example is FPV in the host cell without undergoing homologous recombination with the poxvirus DNA. The helper virus is unable to replicate in the host cell. Furthermore, the helper virus itself is not a component of the reconstituted poxvirus, e.g., FPV for the purposes of this application. In certain embodiments, cowpox virus, Shope fibroma virus, or other suitable poxviruses can be used as a helper virus. In certain embodiments, one or more DNA fragments for transfection can be linearized, circularized, or a combination of linearized and circularized DNA fragments. In certain embodiments, one or more DNA fragments for transfection are cloned into a vector such as a plasmid or a BAC and/or maintained in a host cell such as a bacterial cell, e.g., E. coli.

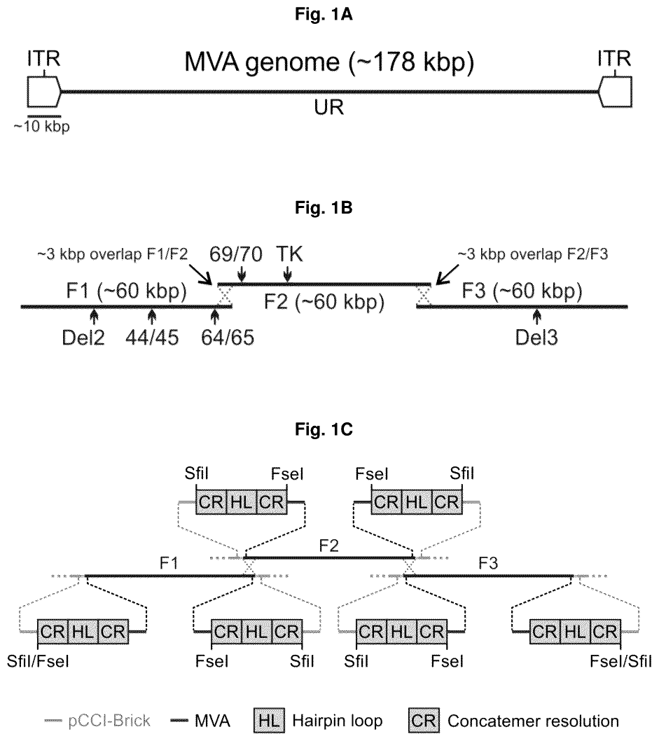

In certain embodiments, one or more DNA fragment(s) further comprise one or both of the inverted terminal repeat (ITR) regions of the poxvirus. In certain embodiments, the sequence of the ITR may contain one or more alterations or variations, which do not affect the design scheme of the reconstituted poxvirus. In one embodiment, the one or more DNA fragments that reconstitute the poxvirus may contain only parts of the ITR sequences. For example, the sMVA fragments F1 and F3 as well as the reconstituted sMVA vectors or recombinant sMVA vectors (FIG. 1-4) may contain only parts of the MVA ITR sequences. In certain embodiments, one or more DNA fragment(s) are further modified to add a poxvirus terminal hairpin loop (HL) sequence at one end or both ends as a duplex copy (double stranded DNA) or the 5′ and/or 3′ ends as a single stranded nucleotide sequence. For example, the HL sequence can be added to either end or both ends of a synthetic DNA fragment as double-stranded or single-stranded DNA sequence in sense (5′→3′) or anti-sense (3′→5′) orientation. In certain embodiments, one or more DNA fragment(s) are further modified to add a poxvirus concatemeric resolution (CR) sequence at one end or both ends as double stranded DNA sequence or at the 5′ and/or 3′ ends as a single stranded nucleotide sequence. The CR sequence can be based on any sequence derived from the consensus sequence 5′-T₆-N₇₋₉-T/C-A₃-T/A-3′, wherein A is adenine, C is cytosine, G is guanine, T is thymine, and N is any nucleotide. In certain embodiments, two CR sequences can be added to both ends of the HL sequence to form a CR-HL-CR sequence. The CR-HL-CR sequence can be added to one or more DNA fragment(s) to one end or both ends as double stranded DNA sequences, or to the 5′ end, 3′ end, or both ends as single stranded nucleotide sequences. In other embodiments, only the HL or CR sequence is added to one end or both ends of one or more DNA fragment(s) as double or single stranded DNA sequence. In certain embodiments, all the DNA fragments contain the HL or CR sequence or a combination thereof at one or both ends. In other embodiments, not all the DNA fragments contain the HL or CR sequence. In certain embodiments, only a subset or a subpopulation of the DNA fragments contain the HL or CR sequence, or a combination thereof. The ITR, HL or CR sequences maybe derived from MVA (NCBI accession #U94848, #AY603355), Vaccinia virus (#NC_006998, #LT966077), Camelpox virus (#NC_003391), Cowpox virus (#NC_003663) Ectromelia virus (#NC_004105), Monkeypox virus (#NC_003310), Racoonpox virus (#NC_027213), Skunkpox virus (#NC_031038), Taterapox virus (#NC_008291), Variola virus (#NC_001611, #L22579), Velopox virus (#NC_031033), Canarypox virus (#NC_005309), Swinepox virus (#NC_003389), FPV (#NC_002188, #MH734528), Myxoma virus (#GQ409969), Sheeppox virus (NC_004002), Goatpox virus (#NC_004003), (Orf virus #NC_005336), Rabbit fibroma virus (#NC_001266), any strain variations of these poxviruses, or any other poxvirus or strain variation thereof.

In certain embodiments, the HL or CR sequences used herein are disclosed as follows:

Sequence of terminal CR-HL-CR sequences containing a duplex copy of the MVA terminal hairpin loop (HL) flanked by MVA concatemeric resolution sequences (215 bp in length, 5′→3′) (SEQ ID NO: 1), wherein the CR sequences are underlined and the HL sequence is italicized and double underlined:

tttttttctaaacactaaatagtaagattaaattaattataa
aattatgtatataatattaattataaaattatgtatatgatttact
aactttagttagataaattaataatacataaattttagtatattaa
tattataaattaataatacataaattttagtatattaatattatat
ttttaaatatttatttagtgtctagaaaaaaa

Sequence of terminal CR-HL-CR sequences containing a duplex copy of the complementary form of the MVA terminal hairpin loop (HL) flanked by concatemeric resolution sequences (215 bp in length, 5′->3′) (SEQ ID NO: 2), wherein the CR sequences are underlined and the HL sequence is italicized and double underlined:

tttttttctagacactaaataaatatttaaaatataatatt
aatatactaaaatttatgtattattaatttataatattaat
atactaaaatttatgtattattaatttatctaactaaagtt
agtaaatcatatacataattttataattaatattatataca
taattttataattaatttaatcttactatttatttagtgtc
tagaaaaaaa

Sequence of the MVA terminal hairpin loop (HL, 165 nt in length, 5′->3′) (SEQ ID NO: 3):

gtaagattaaattaattataaaattatgtatataatattaattataaaa
ttatgtatatgatttactaactttagttagataaattaataatacataa
attttagtatattaatattataaattaataatacataaattttagtata
ttaatattatattttaaa

Sequence of the complementary form of the MVA terminal hairpin loop (HL, 165 nt in length, 5′->3′) (SEQ ID NO: 4):

tttaaaatataatattaatatactaaaatttatgtattattaatttata
atattaatatactaaaatttatgtattattaatttatctaactaaagtt
agtaaatcatatacataattttataattaatattatatacataatttta
taattaatttaatcttac

Sequence of terminal CR-HL-CR sequences containing a duplex copy of the Vaccinia terminal hairpin loop (HL; S-Form) flanked by concatemeric resolution sequences (154 bp in length, 5′->3′) (SEQ ID NO: 5), wherein the CR sequences are underlined and the HL sequence is italicized and double underlined:

ttttttttctagacactaaataaaatattaaaataatattaatgtactaa
aacttatatattattaatttatctaactaaagttagtaaattatatatat
aattttataattaatttaattttactaattttatttagtgtctagaaaaa
aa

Sequence of terminal CR-HL-CR sequences containing a duplex copy of the Vaccinia terminal hairpin loop (HL; F-Form) flanked by concatemeric resolution sequences (154 bp in length, 5′->3′) (SEQ ID NO: 6), wherein the CR sequences are underlined and the HL sequence is italicized and double underlined:

tttttttctagacactaaataaaattagtaaaattaaattaattataaaat
tatatatataatttactaactttagttagataaattaataatatataagtt
ttagtacattaatattatattttaaatattttattagtgtctagaaaaaaa

Sequence 1 of the Vaccinia virus terminal hairpin loop (HL, S-form, 104 nt in length, 5′→3′) (SEQ ID NO: 7):

tagtaaaattaaattaattataaaattatatatataatttactaacttt
agttagataaattaataatatataagttttagtacattaatattatatt
ttaaat

Sequence 2 of the Vaccinia virus terminal hairpin loop (HL, F-form, 104 nt in length, 5′→3′) (SEQ ID NO: 8):

atttaaaatataatattaatgtactaaaacttatatattattaatttat
ctaactaaagttagtaaattatatatataattttataattaatttaatt
ttacta

Sequence 1 of the MVA concatemer resolution sequences (CR, 20 bp in length, 5′→3′) (SEQ ID NO: 9, sense orientation), included at the left end of the hairpin duplex copy in the working example:

tttttttctagacactaaat

Sequence 2 of the MVA concatemer resolution sequences (CR, 20 bp in length, 5′→3′) (SEQ ID NO: 10, antisense orientation), included at the right end of the hairpin duplex copy in the working example:

atttagtgtctagaaaaaaa

As demonstrated in the working examples, sMVA and rsMVA recombinants disclosed herein are generated based on chemical synthesis of three ˜60 kbp long DNA fragments that encompass the entire ˜178 kbp of the MVA genome published by Antoine and colleagues (Accession #U94848) 26. This includes the internal unique region (UR) and the flanking ˜9.6 kbp long inverted terminal repeat (ITR) regions as illustrated in FIG. 1. The MVA genome sequence published by Antoine et al. (Accession #U94848) 26, herein referred to as MVA strain Antoine, differs precisely in five base pairs in the internal UR from the MVA genome of the licensed and commercially available National Institute of Health clone 1 from 1974 (MVA NIH clone 1), which is identical in sequence to the published genome of MVA strain Acambis (Accession #AY603355). sMVA fragment 1 (F1) encompasses the left ITR and ˜50 kbp of the left end of internal UR of the MVA genome; sMVA fragment 2 (F2) contains ˜60 kbp of the middle part of the internal UR of the MVA genome; and sMVA fragment 3 (F3) encompasses ˜50 kbp of the right end of the internal UR and the right ITR of the MVA genome (FIG. 1). sMVA F1 and F2 as well as sMVA F2 and F3 are designed to share ˜3 kbp overlapping sequences to allow the reconstitution of the complete MVA genome by homologous recombination (FIG. 1). A duplex copy of the 165-nucleotide long MVA terminal HL flanked by MVA CR sequences is added to both ends of each of the three fragments to promote MVA genome resolution and packaging²⁶ (FIG. 1). In addition, Sfil and Fsel restriction sites are included at both ends of each of the three fragments in a way that the fragments can be released or linearized with or without the HL and CR sequences. The sole exception is the HL and CR sequences at the ITR of F1 and F3 that are fused to the ends in identical arrangement as it occurs at the junctions of MVA concatemeric replication intermediates²⁶ (FIG. 1). The three sMVA fragments with flanking HL and CR sequences as well as overlapping homologous recombination sequences as depicted in FIG. 1 were synthesized and generated using a yeast-based recombination system by Genscript Biotech. All three sMVA fragments were cloned into a yeast shuttle vector, termed pCCI-Brick, which contains a bacterial mini-F replicon element that can be used as a BAC vector to stably propagate the three fragments at low copy number in bacteria (FIG. 1). The sMVA fragments were ultimately cloned into recombination defective DH10B or EPI300 E. coli cells.

In certain embodiments, a duplex copy of the HL with flanking CR sequences at both ends of each of the three MVA fragments is included—as opposed to only at the ends at the ITR of F1 and F3 where they occur naturally in concatemeric replication intermediates. This design is based on the intrinsic functions that these sequence elements have during poxvirus DNA replication. The terminal HL in packaged poxvirus genomes connects the two DNA strands at the genomic termini to a continuous polynucleotide chain, where they exist at both ends in inverted and complementary forms that are incompletely base-paired and AT-rich²⁶⁻²⁹. Poxvirus HL sequences are important for the replication of the double-stranded DNA genomes into multimeric head-to-tail or head-to-head concatemeric replication intermediates, in which the HL sequences are present at the concatemeric junctions as precise duplex copies³⁰⁻³². The CR elements are comprised of a highly conserved poxvirus resolution sequence and can be found in packaged genomes at both ends of the large ITRs directly adjacent to the terminal HL^{26,27,33,34} Poxvirus CR elements in concatemeric replication intermediates are present on either site of the HL duplex copies at the genomic junctions, and these CR/HL/CR sequence arrangements are essential for the resolution of unit-length genomes and subsequent genome packaging³³⁻³⁶. When circular plasmids containing poxvirus concatemeric junctions, which are composed of an HL duplex copy flanked by CR elements, are transfected into poxvirus infected cells they are spontaneously resolved into linear minichromosomes with terminal HL^{36,37}. In certain embodiments, the circularized sMVA fragments, when transfected into the host cell, can immediately replicate in the host cell in an origin independent manner. It was reported that any circular DNA molecule transfected into poxvirus infected cells is replicated in an origin independent manner, and this non-specific sequence replication is not enhanced by insertion of any viral DNA fragments³⁸. In other embodiments, the circularized sMVA fragments, when transfected into the host cell, can be replicated by a eukaryotic or viral origin of replication inserted into the vector or MVA sequences.

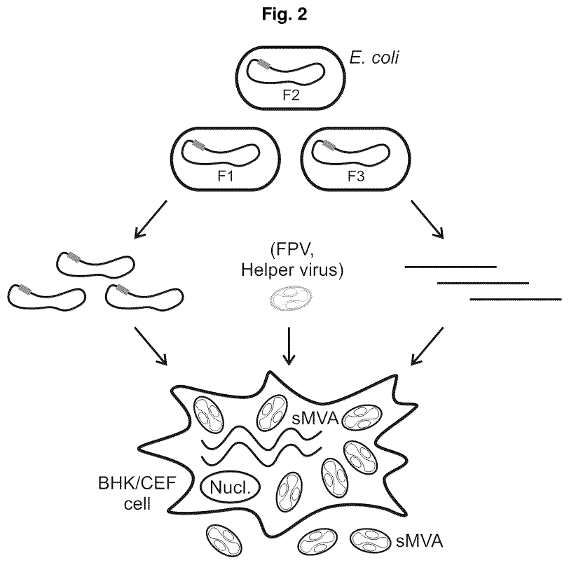

The disclosed construction technique of the synthetic poxvirus such as sMVA includes the HL and CR sequences such that transfection of circular plasmids or DNA molecules containing the three synthetic MVA fragments with flanking concatemeric genomic junctions (CR-HL-CR) and overlapping genome sequences into FPV-infected BHK or CEF cells will promote (1) the transcription and replication of the three sMVA fragments, (2) the resolution of the three sMVA fragments from the plasmid vector sequences, and (3) the recombination of the three sMVA fragments into full-length genomes, ultimately leading to the packaging of vector-free genomes with terminal HL into preformed virus particles (FIGS. 1 and 2). These events may be initiated immediately as a consequence of the FPV infection or they may occur in a step-wise process following transcription initiation and replication of the three sMVA fragments. In another embodiment, the reconstitution process of the sMVA from the three sMVA fragments may be initiated without FPV or other helper virus. In an alternative to this strategy to reconstitute sMVA from the three MVA fragments using circular plasmids or DNA molecules, the reconstitution of sMVA may also be promoted by an approach based on linearized forms of the three sMVA fragments using for example the added Fsel and Sfil restriction sites as shown in FIGS. 1 and 2.

Regardless of whether using circular or linearized forms of the three MVA fragments or a combination of circular and linearized forms, the ends of the fragments may or may not contain HL and CR sequences. In certain embodiments, only a subset of the fragments may contain the HL and CR sequences, or they may be added to only one end or two ends of the fragments as single or double stranded DNA sequences in sense or antisense orientation, for example in a way that they are only present at the MVA partial sequences of F1 and F3 where they occur naturally in putative concatemeric replication intermediates. In another example, not all F1 fragments contain the HL and CR sequences; rather, F1 fragments with or without the HL and CR sequences may be mixed in the construction process. Likewise, not all F2 or F3 fragments are required to contain the HL and CR sequences but a subset or a subpopulation of F2 or F3 fragments may contain the HL and CR sequences. In certain embodiments, HL and/or CR sequences may also be chemically ligated as single or double stranded DNA sequences in linearized forms of the three fragments.

In another embodiment, the sMVA virus reconstituted from the sMVA fragments may be modified by introducing insertions, deletions, or point mutations, or by insertion with one or more heterologous DNA sequences encoding one or more antigens, subunits or fragments thereof. These modifications or antigen sequences may be introduced into the sMVA DNA fragment by conventional transfection/infection methods using a transfer plasmid with homology flanks that mediate homologous recombination. In certain embodiments, the one or more nucleotide sequences encoding the one or more antigens, subunits or fragments thereof may be codon optimized for eukaryotic or vaccinia expression. For example, the antigens, subunits or fragments thereof may be optimized for stability in transcription or expression in the host cell. Various codon optimization techniques may be used, including but not limited to the alteration of four of the same nucleotides in a row (e.g. GGGG, CCCC, TTTT, AAAA) by introducing silent point mutations that do not lead to amino acid changes in the encoded protein, or the adaption of the codon usage to a specific host species.

In another embodiment, the sMVA virus reconstituted from the sMVA fragments in a host cell such as BHK or CEF cells may be used to generate an sMVA bacterial artificial chromosome (BAC) containing a full length sMVA genome. The BAC vector sequences may be inserted into the sMVA genome by a transfer construct containing the BAC sequences with flanking homology sequences that mediate homologous recombination. Circular replication intermediates with inserted BAC sequences may be isolated from host cells such as BHK or CEF cells and transferred by electroporation or chemical transformation into E. coli cells that allow stable propagation of large DNA constructs, such as DH10B or EPI300. In another embodiment, the sMVA BAC may be transferred into GS1783 E. coli cells and manipulated by Red-recombination techniques such as En passant mutagenesis.

In another embodiment, the sMVA fragments may be used to reconstitute a complete or full-length sMVA genome by in vitro ligation methods or by other in vitro DNA assembly methods such as Gibson or Golden Gate assembly.

### Expression of Antigens or Other Heterologous Gene Sequences

The technology disclosed herein has the flexibility of inserting various antigens, subunits or fragments thereof, or other heterologous DNA sequences into the one or more naturally derived or chemically synthesized DNA fragment(s) before transfection such that upon reconstitution, a synthetic poxvirus expressing the antigens, subunits or fragments thereof is obtained. These antigens, subunits or fragments thereof maybe derived from or based on viruses such as cytomegalovirus (CMV), Epstein-Barr virus (EBV), Kaposi-Sarcoma-associated herpesviruses (KSHV), other herpesviruses, Zika virus, Lassa virus, Hepatitis C virus (HCV), Hepatitis (HBV), Coronaviruses (such as 2019-nCOV, SARS, MERS), Influenza, or any other viral, bacterial, or other forms of infectious pathogen. The antigen sequences may also be derived from or based on cancer-associated proteins (e.g., p53, Retinoblastioma, neoantigens). Likewise, other heterologous gene sequences may be inserted into the naturally derived or chemically synthesized poxvirus DNA fragment(s). Such heterologous gene sequences include but are not limited to fluorescence markers, cDNA copies of RNAs such as RNAi, shRNA, LNCRNA, miRNA, etc., interferons, cytokines, antibodies or fragments thereof, or other proteins expressed in prokaryotic or eukaryotic cells. In certain embodiments, the antigen sequences or heterologous gene sequences maybe inserted into the sMVA fragments with an upstream natural or synthetic poxvirus promoter (pSyn, P11, H5, mH5, P28, ATI, pHyb, p7.5) and a downstream transcription termination signal (TTTTTAT), such that the antigen sequences or heterologous gene sequences are expressed when the poxvirus fragments are transfected into a host cell.

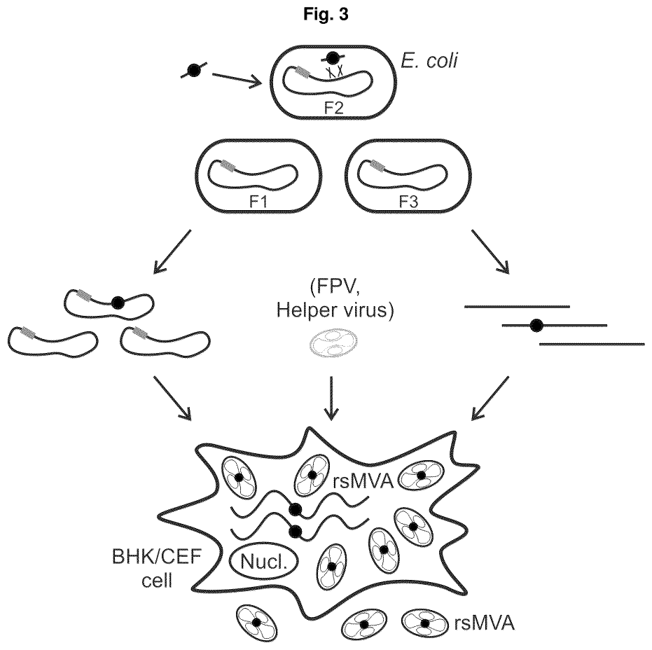

The DNA sequences of the antigens, subunits or fragments thereof, or other heterologous gene sequences, can be inserted into one or more poxvirus insertion sites within the one or more DNA fragment(s). Using sMVA as an example, the DNA sequence of one antigen or fragment thereof can be inserted in a single MVA insertion site located on one sMVA DNA fragment, e.g., sMVA F2, before transfection of the host cell (FIG. 3). In another example, the DNA sequences of two or more antigens or fragments thereof can be inserted into a single MVA insertion site located on one DNA fragment, e.g., sMVA F2, before transfection of the host cell. These antigen DNA sequences may be under the control of a single promoter or two or more promoters. Likewise, the antigen DNA sequences may share the same transcription termination signal or have different transcription termination signals. The nucleotide sequences encoding two or more antigens may also be linked by 2A sequences of picornaviruses (P2A, T2A, F2A, etc.) mediating ribosomal skipping or by internal ribosomal entry sites (IRES) such that the antigens are processed following translation and self-assembled to form a multi-component antigen complex.

In certain embodiments, one or more DNA sequences encoding one or more antigens, subunits or fragments thereof, or other heterologous DNA sequences are inserted in frame at the 5′ end, 3′ end, or any internal position of one or more essential or non-essential poxvirus open reading frames (ORFs) such that when the one or more naturally-derived or chemically synthesized poxvirus DNA fragments are transfected into the host cell, one or more fusion proteins composed of a poxvirus protein with one more antigens, subunits or fragments thereof, or other heterologous protein sequences added to the C-terminus, N-terminus, or any internal position of the poxvirus proteins are expressed. In some embodiments, the one or more DNA sequences encoding one or more antigens, subunits or fragments thereof, or other heterologous DNA sequences are linked to the one or more poxvirus ORFs by 2A encoding sequences of picornaviruses (P2A, F2A, T2A etc,) such that the expressed one or more fusion proteins composed of a poxvirus protein with one more antigens, subunits or fragments thereof, or other heterologous protein sequences added to the C-terminus, N-terminus, or any internal position of the poxvirus proteins are processed (“cleaved”) into the individual components at the 2A linker sequences by a ribosomal skipping mechanism. In other embodiments, the one or more DNA sequences encoding one or more antigens, subunits or fragments thereof, or other heterologous DNA sequences are linked to one or more essential or non-essential poxvirus ORFs by internal ribosomal entry site sequences such that when the one or more naturally-derived or chemically synthesized poxvirus DNA fragments are transfected into the host cell, one or more poxvirus proteins and one more antigens, subunits or fragments thereof, or other heterologous protein sequences are simultaneously expressed through chimeric polycistronic expression constructs with multiple translation initiation sites at the 5′ end of each of the ORFs within the expression constructs.

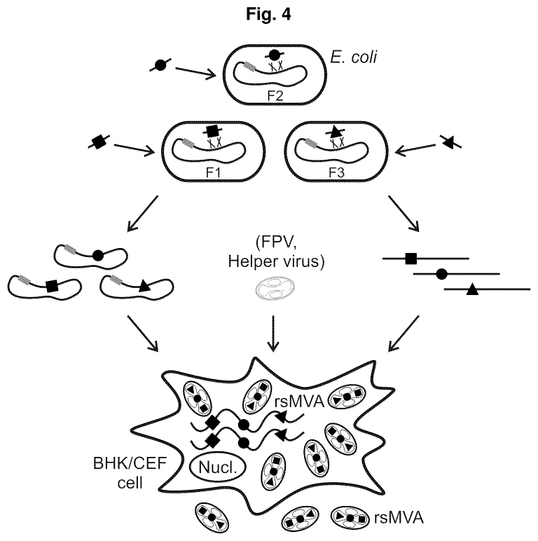

In certain embodiments, the DNA sequences of two or more antigens, subunits or fragments thereof or other heterologous gene sequences may be inserted into two or more MVA insertion sites, which may be located on the same sMVA fragment or on different sMVA fragments. For example, the DNA sequences of two or more antigens, subunits or fragments thereof can be inserted in two different MVA insertion sites, both located on sMVA F1. In another example, the DNA sequences of two or more antigens, subunits or fragments thereof can be inserted into two different MVA insertion sites, one located on sMVA F1 and the other located on sMVA F2 (FIG. 4). It is understood that different DNA fragments may be inserted with the DNA sequence of the same antigen, subunits or fragments thereof. Alternatively, different sMVA fragments may be inserted with the DNA sequences of different antigens, subunits or fragments thereof such that upon reconstitution after transfection, the different antigens, subunits or fragments thereof expressed by the synthetic MVA are self-processed and self-assembled into a viral particle, a viral complex, or a complete antigen comprising the subunits or fragments thereof.

As demonstrated in the working examples, previously established methods of generating recombinant MVA vectors using BAC technology^{16,18,24,25} were adapted to generate rsMVA recombinants using the three sMVA fragments. Because the three sMVA fragments have been cloned into a BAC vector with mini-F replicon they can be stably propagated in bacteria and, consequently, they can be manipulated by highly efficient recombination methods such as En Passant mutagenesis or other E. coli-based manipulation procedures^{39,40} Using these methods, naturally derived or synthetic heterologous antigen sequences together with an upstream vaccinia virus promoter and downstream transcription termination signal can be inserted into only one, two, or all three of the sMVA fragments in a parallel, successive, or repeated manner into virtually every MVA genome position (FIGS. 3 and 4). These insertion sites may include commonly used insertion sites such as the MVA deletion 2 (Del2) site, the intergenic region (IGR) between open reading frame (ORF) 44L and 45L (IGR44/45), the IGR between ORF 69R and 70L (IGR69/70), the IGR between 64L and 65L (IGR64/65), the Thymidine Kinase (TK) gene insertion site, or the MVA Deletion 3 (Del3) site (FIG. 1), or any other MVA deletion site, intergenic region, or gene insertion site (ORF numbers are based on MVA strain Antoine (Accession #U94848)). These methods may also be used to generate one or multiple point mutations, insertions, or deletions at one or multiple positions of one or more of the sMVA fragments. The modified sMVA fragments can be then isolated from E. coli and co-transferred as circular or linearized DNA molecules by various transfection methods into BHK or CEF cells infected with a helper virus to initiate the reconstitution of rsMVA with single, double, or multiple antigen insertions or genome alterations (FIGS. 3 and 4). In certain embodiments, the rsMVA reconstitution can be initiated without the addition of a helper virus. In certain embodiments, the three sMVA fragments may also be used to generate sMVA with single or multiple antigen insertion or genome alterations by in vitro ligation methods. In certain embodiments, one or more antigens, subunits or fragments thereof can be inserted into one or more MVA insertion sites of the sMVA fragments. In certain embodiments, only one sMVA fragment is inserted with one or more antigens or fragments thereof (FIG. 3). In certain embodiments, two or more sMVA fragments are inserted with one or more antigens or fragments thereof (FIG. 4). In certain embodiments, all sMVA fragments are inserted with one or more antigens, subunits or fragments thereof (FIG. 4). When two or more sMVA fragments are inserted with antigens, subunits or fragments thereof, the antigens, subunits or fragments thereof may be the same or different for different sMVA fragments. For example, sMVA F1 may be inserted with one type of antigen or fragment thereof, and sMVA F2 may be inserted with a different type of antigen or fragment thereof. Alternatively, sMVA F1 and F2 may be inserted with the same type of antigen or fragment thereof and sMVA F3 may be inserted with the same type of antigen or fragment thereof or with a different type of antigen, subunit or fragment thereof.

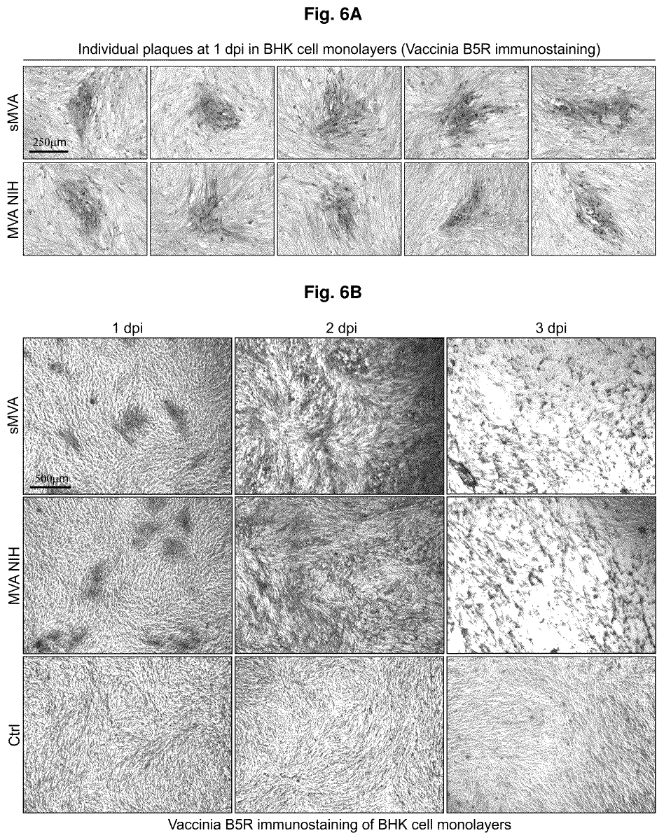

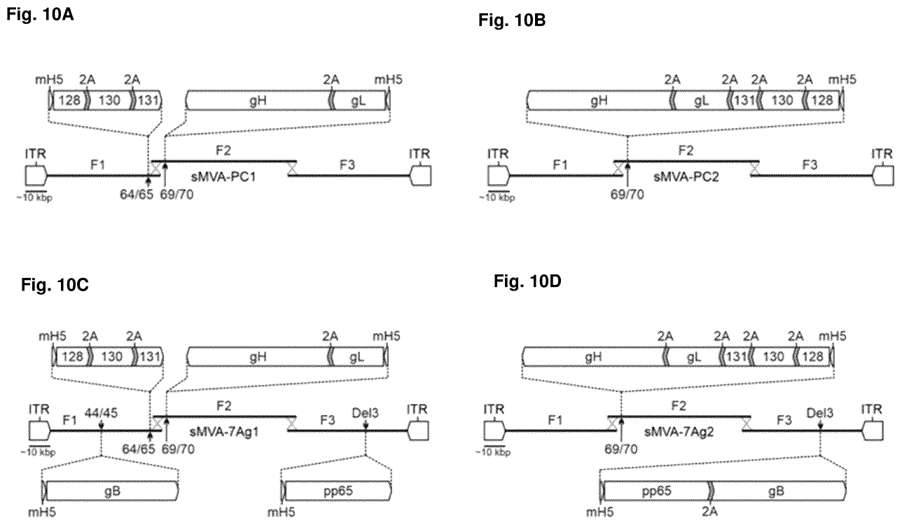

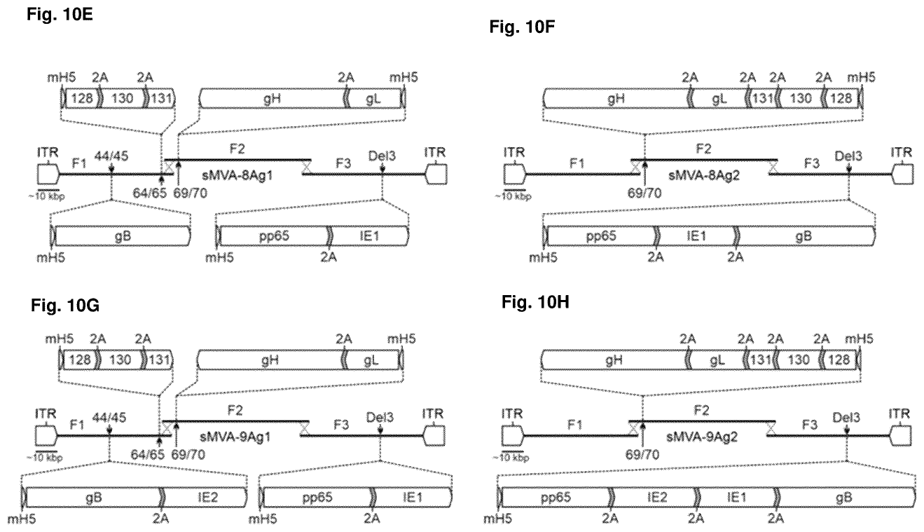

The poxvirus vectors produced by the technology disclosed herein may be used, for example, to generate multi-antigenic vaccine vectors to stimulate polyfunctional humoral and cellular immune response against various conditions such as viral infections and cancer. As an example, the disclosed technology based on the three sMVA fragments F1-F3 may be used to generate multi-antigenic rsMVA vaccine vectors to stimulate polyfunctional humoral and cellular immune response against human cytomegalovirus (HCMV). This may include immunodominant antigen sequences based on the five subunits of the HCMV pentamer complex (PC), glycoprotein B (gB), phosphoprotein 65 (pp65), and the immediate-early 1 and 2 proteins (IE1 and IE2). The antigen sequences may be inserted separately or combined as 2A-linked polycistronic expression constructs into the sMVA fragments at different commonly used MVA insertion sites (Del2, Del3, IGR44/45, IGR69/70, IGR64/65) to generate rsMVA vaccine vectors expressing 5, 6, 7, 8, or 9 HCMV antigens, as illustrated in FIG. 6 and FIG. 10. rsMVA vectors expressing only single HCMV antigens or any other number or combination of the above-mentioned nine HCMV antigens may also be generated. All antigen sequences or expression constructs may be inserted together with an upstream mH5 promoter and downstream transcription termination signal. These vectors may be used to stimulate immune responses against HCMV in animal models or humans.

### Expression Systems and Vaccines

According to the embodiments described herein, an MVA expression system is provided herein. In certain embodiments, the expression system may express one or more desired antigens, subunits or fragments thereof or other heterologous protein sequences.

As described above, one or more sMVA fragments may be inserted with the DNA sequences encoding one or more antigens, subunits or fragments thereof such that the reconstituted sMVA simultaneously expresses the antigens, subunits and fragments thereof.

In certain embodiments, the antigen DNA sequences inserted into the sMVA fragments may be based on the natural DNA sequence or from chemical synthesis. In other embodiments, the antigen DNA sequences may be optimized for expression and stability within the expression system.

The sMVA described herein may be part of a vaccine composition that may be used in methods to treat or prevent viral infection or to treat cancer, depending on the antigens expressed by the sMVA. The vaccine composition as described herein may comprise a therapeutically effective amount of the sMVA as described herein, and further comprising a pharmaceutically acceptable carrier according to a standard method. Examples of acceptable carriers include physiologically acceptable solutions, such as sterile saline and sterile buffered saline.

In some embodiments, the vaccine or pharmaceutical composition may be used in combination with a pharmaceutically effective amount of an adjuvant to enhance the prophylactic or therapeutic effects. Any immunologic adjuvant that may stimulate the immune system and increase the response to a vaccine, without having any specific antigenic effect itself may be used as the adjuvant. Many immunologic adjuvants mimic evolutionarily conserved molecules known as pathogen-associated molecular patterns (PAMPs) and are recognized by a set of immune receptors known as Toll-like Receptors (TLRs). Examples of adjuvants that may be used in accordance with the embodiments described herein include Freund's complete adjuvant, Freund's incomplete adjuvant, double stranded RNA (a TLR3 ligand), LPS, LPS analogs such as monophosphoryl lipid A (MPL) (a TLR4 ligand), flagellin (a TLR5 ligand), lipoproteins, lipopeptides, single stranded RNA, single stranded DNA, imidazoquinolin analogs (TLR7 and TLR8 ligands), CpG DNA (a TLR9 ligand), Ribi's adjuvant (monophosphoryl-lipid A/trehalose dicorynoycolate), glycolipids (α-GalCer analogs), unmethylated CpG islands, oil emulsion, liposomes, virosomes, saponins (active fractions of saponin such as QS21), muramyl dipeptide, alum, aluminum hydroxide, squalene, BCG, cytokines such as GM-CSF and IL-12, chemokines such as MIP 1-α and RANTES, activating cell surface ligands such as CD40L, N-acetylmuramine-L-alanyl-D-isoglutamine (MDP), and thymosin α1. The amount of adjuvant used can be suitably selected according to the degree of symptoms, such as softening of the skin, pain, erythema, fever, headache, and muscular pain, which might be expressed as part of the immune response in humans or animals after the administration of this type of vaccine.

In further embodiments, use of various other adjuvants, drugs or additives with the vaccine of the invention, as discussed above, may enhance the therapeutic effect achieved by the administration of the vaccine or pharmaceutical composition. The pharmaceutically acceptable carrier may contain a trace amount of additives, such as substances that enhance the isotonicity and chemical stability. Such additives should be non-toxic to a human or other mammalian subject in the dosage and concentration used, and examples thereof include buffers such as phosphoric acid, citric acid, succinic acid, acetic acid, and other organic acids, and salts thereof; antioxidants such as ascorbic acid; low molecular weight (e.g., less than about 10 residues) polypeptides (e.g., polyarginine and tripeptide) proteins (e.g., serum albumin, gelatin, and immunoglobulin); amino acids (e.g., glycine, glutamic acid, aspartic acid, and arginine); monosaccharides, disaccharides, and other carbohydrates (e.g., cellulose and derivatives thereof, glucose, mannose, and dextrin), chelating agents (e.g., EDTA); sugar alcohols (e.g., mannitol and sorbitol); counterions (e.g., sodium); nonionic surfactants (e.g., polysorbate and poloxamer); antibiotics; and PEG.

The vaccine or pharmaceutical composition containing the sMVA described herein may be stored as an aqueous solution or a lyophilized product in a unit or multiple dose container such as a sealed ampoule or a vial.

### Preventing or Treating Viral Infections or Cancer

The poxvirus reconstituted from the one or more naturally derived or chemically synthesized DNA fragment(s), with or without the inserted antigens, subunits or fragments thereof, or other heterologous sequences may be used as a vaccine composition for preventing or treating various viral infections or cancer. One of ordinary skill in the art would know how to select a particular viral or cancer antigen for the conditions or diseases to be prevented or treated. This may include but is not limited to any infectious disease or cancer antigen that is capable of eliciting an immune response, such as viral envelope glycoproteins or glycoprotein complexes, immunodominant T cell antigens, or mutated cancer neoantigens. These antigen sequences or portions thereof maybe derived from or based on viruses such as CMV, EBV, KSHV, other herpesviruses, Zika virus, Lassa virus, HCV, HBV, Coronaviruses, Influenza, or any other viral, bacterial, or other forms of infectious pathogen.

The following examples are intended to illustrate various embodiments of the invention. As such, the specific embodiments discussed are not to be constructed as limitations on the scope of the invention. It will be apparent to one skilled in the art that various equivalents, changes, and modifications may be made without departing from the scope of invention, and it is understood that such equivalent embodiments are to be included herein. Further, all references cited in the disclosure are hereby incorporated by reference in their entirety, as if fully set forth herein.

### EXAMPLES

### Example 1: Reconstitution of sMVA Using Circular Forms of the Synthetic MVA Fragments

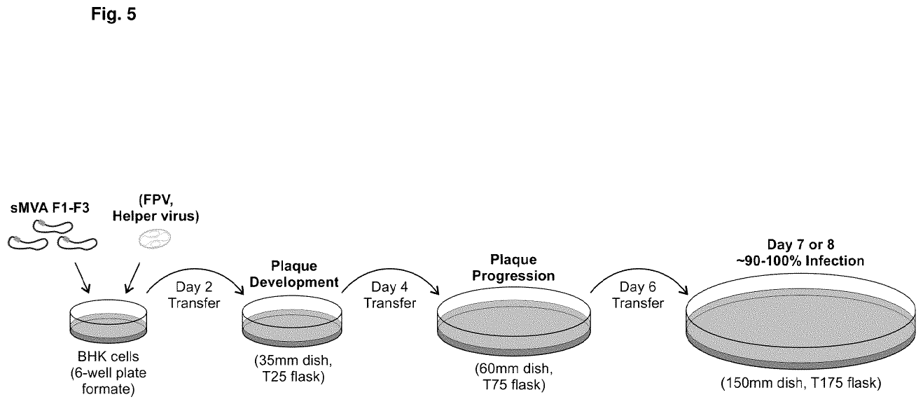

As an initial test to validate the sMVA platform, a procedure as illustrated in FIG. 2 was used to evaluate the reconstitution of sMVA from circular forms of the three sMVA fragments. The three sMVA fragments (F1 to F3) were isolated by alkaline lysis⁴¹ from EPI300 or DH10B E. coli, which allows stable propagation of large DNA fragments cloned as a BAC, and co-transfected (˜1-2 μg/fragment) as circular plasmids by lipofection into monolayers of BHK cells (6 well plate format) (FIG. 5). At 4 hours post infection (hpi), the BHK cells were infected with a helper FPV at 0.1 multiplicity of infection (MOI) to initiate MVA transcription and DNA replication. FPV-infected BHK cells transfected with only one or a combination of only two of the MVA fragments, mock-infected (uninfected) BHK cells transfected with all three MVA fragments, and mock-transfected BHK cells infected with FPV were used as controls. Transfected/infected BHK cells were grown under appropriate conditions and every 2 days dispersed (passaged) in ˜1:2 to 1:3 ratios into larger tissue culture formats using a procedure as illustrated in FIG. 5. Cytopathic effects (CPE) and signs of viral plaque formation and progression demonstrating reconstitution of sMVA were detected in FPV-infected BHK cell monolayers transfected with all three synthetic MVA fragments at 3-6 days post transfection/infection (dpt/i). At 7 to 8 dpt/i following dispersion of the FPV-infected BHK cells transfected with all three MVA fragments, the derived BHK cell monolayers showed 90-100% infection. In contrast, CPE or signs of characteristic MVA viral plaque formation were not observed in any of the controls. sMVA was prepared from BHK cell monolayers with 100% CPE by conventional freeze/thaw method, titrated on BHK cells, and used to infect BHK cell monolayers at low MOI to evaluate MVA infection and spreading at 16-72 hpi by immunostaining for the vaccinia virus glycoprotein B5R^{16,42}.

This analysis indicated that the reconstituted sMVA had similar capacity compared to MVA NIH clone 1 to form characteristic MVA viral foci and to spread in BHK cell monolayers (FIG. 6), confirming the formation of sMVA from the three sMVA fragments. Characteristic viral foci were detected in sMVA and MVA NIH-infected BHK cell monolayers at 1 day post infection (dpi), while at 2 dpi MVA viral infection was visible in the entire BHK cell monolayers infected with sMVA or MVA NIH, and at 3 dpi most cells of the BHK cell monolayers infected with sMVA or MVA NIH were detached. In contrast, no characteristic MVA viral infection or CPE was detected in mock-infected controls. Other research groups conclusively demonstrated that FPV, used as a helper virus, contributes no genetic material or recombines with the MVA genome when introduced into the transfection/infection incubation on BHK or CEF cells^{43,44}.

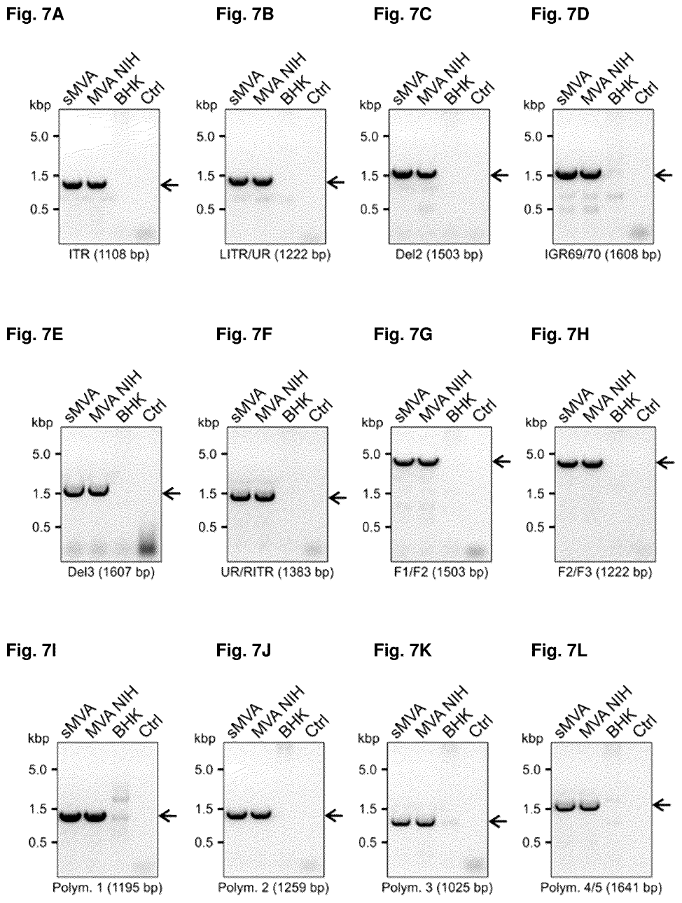

To characterize the genome of the reconstituted sMVA, viral DNA prepared from sMVA-infected BHK cells was evaluated by PCR using primers specific for different genome positions within the three synthetic MVA fragments. DNA prepared from mock-infected (uninfected) BHK cells as well as BHK cells infected with the NIH MVA clone 1 were used as controls. This PCR analysis revealed the presence of DNA derived from all three sMVA fragments within the genomic DNA of the reconstituted sMVA (FIG. 7). All PCR products derived from sMVA-infected BHK cells were similar (in molecular weight) compared to those derived from MVA NIH-infected BHK cells. This included (1) PCR products derived from the ITR sequences located within sMVA F1 and F3 (FIG. 7A); (2) PCR products corresponding to sMVA F1-derived DNA such as the transition of the left ITR into the left end of the internal UR (FIG. 7B), and the Del2 insertion site (FIG. 7C); (3) PCR products corresponding to sMVA F2-derived DNA such as the G1L/18R insertion site (IGR69/70) (FIG. 7D); and PCR products corresponding to sMVA F3-derived DNA such as the Del3 insertion site (FIG. 7E), the transition of the right end of the internal UR into the right ITR (FIG. 7F). In addition, PCR analysis using primers with binding sites flanking the sMVA F1/F2 or sMVA F2/F3 overlapping homologous sequences (FIG. 1) showed similar product sizes for MVA and MVA NIH-infected BHK cells (FIGS. 7G-7H), indicating successful recombination of the three sMVA fragments and removal of the vector sequences in sMVA.

Sanger sequencing analysis of all PCR products derived from sMVA-infected BHK cells revealed sequences at these genome positions that were identical to the published sequence of MVA strain Antoine²⁶, including the recombination sites of F1/F2 and F2/F3. The only exception to this was the PCR product derived from the Del2 site of sMVA showing a single T to A nucleotide alteration located in the IGR at three base pairs downstream of open reading frame (ORF) 021L²⁶. BLAST analysis showed that this sequence alteration within sMVA DNA is not present in any published MVA or vaccinia virus genome sequence. Sanger sequences analysis of sMVA F1 purified from E. coli indicated that this specific nucleotide alteration at the Del2 site was already present before the sMVA reconstitution in BHK cells, suggesting that it has its origin in the chemical synthesis of F1 or is a result of the cloning or propagation of sMVA F1 in E. coli. Additional sequencing analysis of PCR products (FIGS. 7I-7L) derived from sMVA-infected BHK cells indicated that the sMVA genome sequence contained all five Antoine specific nucleotide polymorphisms in the internal UR that distinguishes it from the genome sequence of the MVA NIH clone 1.

The following DNA sequences are given as the nucleotide sequences (5′ to 3′) of the sense strands of the DNA molecules. Sequence of sMVA fragment 1 (F1, 60021 bp in length) (SEQ ID NO: 11):

ggcctagcaggccggcctttttttctagacactaaataaatagtaagattaaattaattataaaattatgtatataatattaa
ttataaaattatgtatatgatttactaactttagttagataaattaataatacataaattttagtatattaatattataaattaata
atacataaattttagtatattaatattatattttaaatatttatttagtgtctagaaaaaaatgtgtgaccaacgaccgtagga
aactctagagggtaagaaaaatcaatcgctttatagagaccatcagaaagaggtttaatatttttgtgagaccatcgaa
ggagaaagagataaaacttttttacgactccatcagaaagaggtttaatatttttgtgagaccatcgaagagagaaag
agataaaacttttttacgactccatcagaaagaggtttaatatttttgtgagaccatcgaagagagaaagagataaaac
ttttttacgactccatcagaaagaggtttaatatttttgtgagaccatcgaaggagaaagagataaaacttttttacgactc
catcagaaagaggtttaatatttttgtgagaccatcgaaggagaaagagataaaacttttttacgactccatcagaaag
aggtttaatatttttgtgagaccatcgaaggagaaagagataaaacttttttacgactccatcagaaagaggtttaatattt
ttgtgagaccatcgaagagagaaagagataaaacttttttacgactccatcagaaagaggtttaatatttttgtgagacc
atcgaaggagaaagagataaaacttttttacgactccatcagaaagaggtttaatatttttgtgagaccatcgaagaga
gaaagagaataaaaatattttagtgacaccatcagaaagaggtttaatatttttgtgagaccatcgaagagagaaag
agataaaacttttttacgactccatcagaaagaggtttaatatttttgtgagaccatcgaaggagaaagagataaaactt
ttttacgactccatcagaaagaggtttaatatttttgtgagaccatcgaagagagaaagagataaaacttttttacgactc
catcagaaagaggtttaatatttttgtgagaccatcgaagagagaaagagataaaacttttttacgactccatcagaaa
gaccatcgaagagagaaagagaaagagatagttagtctagatatttttcttagtacaaaagtcaatgttttaaaatatat
ggacaagaatttgtctgtataaaaacttgtgtgaaattttgtaccaaagaaaaaatgtgagcagtatcccctacatggat
tttactagatcatttatataccaaaaaatattatacgatctacgttttattatatgattttaacgtgtaaattataaacattatttta
tgatatacaattgtctggtaacctagatgggcataggggatgagtatatgttgttggacgttattgtttaagaaatagttgat
gcatcagaaagaggtttaatatttttgtgagaccatcgaagagagaaagagataaaacttttttacgactccatcagaa
agaggtttaatatttttgtgagaccatcgaagagagaaagagataaaacttttttatgactccattgaagagagaatga
gaataaaaatattttagtgacaccatcagaaagaggtttaatattttttatgagaccatcaaagagagaaagagaata
aaaatattttatgactccattgaagagagaaagagaaaatgagaataaaaatattttagtgacaccatcagaaagag
gtttaatattttttatgagaccatcaaagagagaaagagaataaaaatatttttgtaaaactttttttatgagaccatcaaa
gagagaaagagaataaaaatatttttgtaaaactttttttatgagaccatcaaagagagaaagagaataaaaatattttt
gtaaaactttttttatgagaccatcaaagagagaaagagaataaaaatatttttgtaaaactttttttatgagaccatcaa
agagagaaagagaataaaaatatttttgtaaaactttttttatgagaccatcaaagagagaaagagaataaaaatattt
ttgtaaaactttttttatgagaccatcaaagagagaaagagaataaaaatatttttgtaaaactttttttatgagaccatca
aagagagaaagagaataaaaatattttatgactccattgaagagagaaagagaataaaaatattttagtgacaccat
cagaaagaggtttaatatttttgtgagaccatcgaagagagaaagagaataaaaatattttatgactccattgaagag
agaaagagaataaaaatattttagtgacaccatcagaaagaggtttaatattttttatgagaccatcaaagagagaaa
gagaataaaaatatttttgtaaaactttttttatgagaccatcaaagagagaaagagaataaaaatatttttgtaaaacttt
ttttatgagaccatcaaagagagaaagagaataaaaatatttttgtaaaactttttttatgagaccatcaaagagagaa
agagaataaaaatatttttgtaaaactttttttatgagaccatcaaagagagaaagagaataaaaatatttttgtaaaact
ttttttatgagaccatcaaagagagaaagagaataaaaatattttatgactccattgaagagagaatgagaataaaaa
tattttagtgacaccatcagaaagaggtttaatatttttgtgagaccatcgaagagagaaagagaataaaaatattttat
gactccattgaagagagaaagagaataaaaatattttagtgacaccatcagaaagaggtttaatattttttatgagacc
atcaaagagagaaagagaataaaaatatttttgtaaaactttttttatgagaccatcaaagagagaaagagaataaa
aatatttttgtaaaactttttttatgagaccatcaaagagagaaagagaataaaaatatttttgtaaaactttttttatgagac
catcaaagagagaaagagaataaaaatatttttgtaaaactttttttatgagaccatcaaagagagaaagagaataa
aaatatttttgtaaaactttttttatgagaccatcaaagagagaaagagaataaaaatatttttgtaaaactttttttatgaga
ccatcaaagagagaaagagaataaaaatatttttgtaaaactttttttatgagaccatcaaagagagaaagagaata
aaaatattttatgactccattgaagagagaatgagaataaaaatattttagtgacaccatcagaaagaggtttaatatttt
tgtgagaccatcgaagagagaaagagaataaaaatattttatgactccattgaagagagaatgagaataaaaatatt
ttagtgacaccatcagaaagaggtttaatattttttatgagaccatcaaagagagaaagagaataaaaatatttttgtaa
aactttttttatgagaccatcaaagagagaaagagaataaaaatatttttgtaaaaattataaacattattttatgatatac
aattgtctggtaacctagatgggcataggggatgttgataagctcgacgagtatatgttgttggacgttattgtttaagaa
atagttgatgcatcagaaagagaataaaaaatattttagtgagaccatcgaagagagaaagagataaaacttttttac
gactccatcagaaagaggtttaatatttttgtgagaccatcgaagagagaaagagataaaacttttttacgactccatc
agaaagaggtttaatatttttgtgagaccatcgaaggagaaagagataaaacttttttacgactccatcagaaagaggt
ttaatatttttgtgagaccatcaaagagagaaagagaataaaaatatttttgtaaaactttttttatgagaccatcaaaga
gagaaagagaataaaaatatttttgtaaaactttttttatgagaccatcaaagagagaaagagaataaaaatatttttgt
aaaactttttttatgagaccatcaaagagagaaagagaataaaaatatttttgtaaaactttttttatgagaccatcaaag
agagaaagagaataaaaatattttatgactccattgaagagagaatgagaataaaaatattttagtgacaccatcag
aaagaggtttaatatttttgtgagaccatcgaagagagaaagagaataaaaatattttatgactccattgaagagaga
atgagaataaaaatattttagtgacaccatcagaaagaggtttaatattttttatgagaccatcaaagagagaaagag
aataaaaatatttttgtaaaactttttttatgagaccatcaaagagagaaagagaataaaaatatttttgtaaaacttttttta
tgagaccatcaaagagagaaagagaataaaaatatttttgtaaaactttttttatgagaccatcaaagagagaaaga
gaataaaaatatttttgtaaaactttttttatgagaccatcaaagagagaaagagaataaaaatattttatgactccattg
aagagagaatgagaataaaaatattttagtgacaccatcagaaagaggtttaatatttttgtgagaccatcgaagaga
gaaagagaataaaaatattttatgactccattgaagagagaaagagaataaaaatattttagtgacaccatcagaaa
gaggtttaatattttttatgagaccatcaaagagagaaagagaataaaaatatttttgtaaaactttttttatgagaccatc
aaagagagaaagagaataaaaatattttatgactccattgaagagagaatgagaataaaaatattttagtgacacca
tcagaaagaggtttaatatttttgtgagaccatcgaagagagaaagagaataaaaatattttatgactccattgaagag
agaatgagaataaaaatattttagtgacaccatcagaaagaggtttaatatttttgtgagaccatcgaagagagaaag
agaataaaaatattttatgactccattgaagagagaatgagaataaaaatattttagtgacaccatcagaaagaggttt
aatattttttatgagaccatcaaagagagaaagagaataaaaatatttttgtaaaactttttttatgagaccatcaaagag
agaaagagaataaaaatatttttgtaaaactttttttatgagaccatcaaagagagaaagagaataaaaatatttttgta
aaactttttttatgagaccatcaaagagagaaagagaataaaaatatttttgtgagaccatcaaagagagaaagaga
ataaaaatatttttgtgagaccatcaaagagagaaagagaataaaaatatttttgtgagaccatcaaagagagaaag
agaataaaaatattttatgactccattgaagagagaaagagaataaaaatattttagtgacaccatcagaaagaggtt
taatatttttgtgagaccatcgaagagagaaagagaataaaaatattttatgactccattgaagagagaaagagaata
aaaatattttagtgacaccatcagaaagaggtttaatattttttatgagaccatcaaagagagaaagagaataaaaat
attttttatgagaccatcaaagagagaaagagaataaaaatattttatgagaccatcaaagagagaaagagaataa
aaatattttttatgagaccatcaaagagagaaagagaataaaaatattttatgagaccatcaaagagagaaagaga
ataaaaatattttttatgagaccatcaaagagagaaagagaataaaaatatttttgtatgagaccatcagaaagaggtt
taatatttttgtgataccctgaaaggaaataggaatagtgtcataatcgtatcacactattgagacagaaaaagaagaa
gtcgcgagaggtaactttttgttttgcaaaccggaatatagtgtccggtacacttttttaattcgtggtgtgcctgaatcgttc
gattaaccctactcatccaatttcagatgaatagagttatcgattcagacacacgctttgagttttgttgaatcgatgagtg
aagtatcatcggttgcaccttcagatgccgatccgtcgacatacttgacctcaagttcagatgattccttgcacatgtctcc
gatacgaacgctaaactctagattcttgacacattttgtatcgacgatcgttgaaccgatgatatcttcgtaactcactttctt
atgagagatgttagacccgagtactggatgggtcttgatgtcgctgtctttctcttcttcgctacatctgatgtcgatagaca
cctcacagtctttccatcagcggattctgagatggatttaatctgaggacatttggtgaatccaaagttcattctcagacct
ccaccgatgatggagtaataagtggtaggaggatctacatcctcgactgattccacctcgggatctggatctgactcgg
actctgtaatttccgttacggattggcaaatcttatcatcggtcggtgtttggtcttgctttgtgactttgataataacatcgatt
cccatatgatgtttgttttcttcttccgtacacgatgaggatgattgctgaagactggcaggcacatgcatgccagtacga
tatattgtttcatgattgctattgattgagtactgttctttatgattctacttccttaccgtgcaataaattagaatatattttctactt
ttacgagaaattaattattgtatttatgggtgaaaaacttactataaaaagcgggtgggtttggaattagtgatcagtttatg
tatatcgcaactaccgggcatatggctacattacccacatgataagagattgtatcagtttcgtagtcttgagtattggtatt
actatatagtatatagatgtcgacgctagagttactgtctccgaatgcggcatgatagtatcattctttgctttcgttaactgtt
tggaggaagaatctttgttattgcatttaatctcgaaattcagagtgcacacctttctcctgtaaagaaacctgaagtcgct
accttattaagaagacgggatcgcagtctttatgattcatagtaatagttagttccgacgttgagatggattcgctgagac
cggtagtggtcgtccgagtacacgatgtgtcgttaactggatacaggttaatttccacatcgatatagttaaaggtatttct
gggtacgggttcgcatttatctgcggaagagacggtgtgagaatatgttccgagaccacacggagaacagatgacgt
ctccggatactccgtatcctattccacattttgtttgggaaacacatgccttgcatccatgatcgggagagcattcacaga
ttctattgtgagtcgtgttacacgatcgcgtcgacattgttgacagaaacgtgaccttcattcttaccgtcgtccataaatac
gttaggtatgtaccacatactgtcgcgaacgatgcgtccatctcataatgatttactttttcataattaaagatgtgaaaga
aaaccgaacaatatatttttttagtaatgtttatgcgagacatataaaataaactccgtgtttatgatgccggtaaatgttttt
atcatcttggacggaatcgattttgtaatatgccatggaaacaggacattatcactccatgataaattatttaatggagtc
gatcctctcattgttctttgcgtatctcaatctgtggcgtttgcttcgtttaaataatatatcaaacatggagacgcctgatatg
taggcattcttcattctattaatgtctgctctatagcgctttagttccttatgacgaccggcgatatcatacttactttagaagg
aaaatcatcatctaggattaaggcgtatctgatacaggcgaataatggttcaggatatagatagcgtatatctctattaa
atgcgtcaatcatagtctctagagtgggatggtaactcagtaataaatcaactagcttctctttggtaactgcttttctggat
ggccgtattgattatcgagcgtgacactcgctccatattccaataaccgctttgcaaattgtatattattgacatcgaccgc
gtaatatagtagagttatcgatcatatctatatcatccatgtacttgcttagtatatcaaatacatcttcataacagtgatacc
cgcaattattaaatctcgataatatcagaccgtacatacatagacggccattgttagatatgtgatttacagccgcgtgtc
catattttccacgataaaccttacgacgtttacatcgacgagattattattaacaaagttgttgtccgtcgtcttatccaacat
gcattgaatgataggtatacttaccatatcgccgtaatgtaagtagtttatcagtatggcttgtacgatggattcatcctgtt
gtctaaatctctttagaatgttatcgatgatgtagtggttatattctctggaatcgtacgaagtaatactacgcattacgtcg
acaagagtatgacgtctctcaataagaagattaacgatttccatgtctacattatatggggttactctaaatcgcttgtttag
ataatacgcctctaatatagggctgacgtcgtatactctacacgtgtccacatcctttattaataatctctatatctatggttg
agcaagaccagtagtattggatggaaacattgttatcgatcaaacatttaattacatccttggatagagattctctatgag
acgatatatagtaatgaagagagttcttacacatatcactgttgtacatacaggtacgaaatacgtaaccggtgctgta
acattctgatttaagaagccatagcaatacttctggtctcggattaggcgtcgttacgtatatatccaccaatccgagacc
attgattgcataattcgtattcttggacggacgtatccgtttatccacaattaggtattttagcagacgtaagtcgaaatcatt
tatattcgacttgagttcgttagaggaattcgaatagctggatatcagtagatgcacaatctgagattttacgtatctatgct
tactgtatgctcctagcggagttaatccttcgttgtttctacaaagtctctcgactccgcgagagagtaacagtcgaacaa
tcttaatgtctgtatcgcatttattggagacgtaacaatgtagcgcattgtttcctcgtctatctatatgttttgataagttgtgac
acgtttcaatttctagttttatttttttgtacgtcacatcttcatccagtagacgacatagaatacatgtgcaatccatagctatt
ctggtgctaattattcctcataagatgataaaaagtgtagtgagagagcatgaaggagatttagtatttagcagtgcgg
atatgatccaagagggtgagatagtcgttctcgttcagaatctttcgcagcataagtagtatgtcgatatacttatcgttga
agactcttccagagacgatagctgattgagtacaaagtccaatgattgcacgaagttcttcggcggttttcatggagtca
tttctgatgaaacatttaatgatctaaatttcagtttatgtttgtaccccgtattcatacttaacaaattggtattacataccatta
ataatgcaagcataaaaaatcgttagtagatgtttctaaatataggttccgtaagcaaagaatataagaatgaagcgg
taatgataaaatcaatcgttatctaaaatgatcatactcatttattttattctattatattaacacatacatttttaacagcaac
acattcaatattgtattgttatttttatattatttacacaattaacaatatattattagtttatattactgaattaataatataaaatt
cccaatcttgtcataaacacacactgagaaacagcataaacacaaaatccatcaaaaatgttgataaattatctgatg
ttgttgttcgctgctatgataatcagatcattcgccgatagtggtaacgctatcgaaacgacatcgccagaaattacaaa
cgctacaacagatattccagctatcagattatgcggtccagagggagatggatattgtttacacggtgactgtatccac
gctagagatatcgacggtatgtattgtagatgctctcatggttatacaggcattagatgtcagcatgtagtattagtagact
atcaacgttcagaaaaaccaaacactacaacgtcatatatcccatctcccggtattatgcttgtattagtaggcattattat
tattacgtgttgtctattatctgtttataggttcactcgacgaactaaactacctatacaagatatggttgtgccataatttttat
aaattttttttatgagtatttttacaaaaatgtataaagtgtatgtcttatgtatatttataaaaatgctaaatatgcgatgtatct
atgttatttgtatttatctaaacaatacctctacctctagatattatacaaaaattttttatttcggcatattaaagtaaaatcta
gttaccttgaaaatgaatacagtgggtggttccgtatcaccagtaagaacataatagtcgaatacagtatccgattgag
attttgcatacaatactagtctagaaagaaatttgtaatcatcttctgtgacgggagtccatatatctgtatcatcgtcccat
gctatattcctgttatcatcattagttaatgaaaataactctcgtgcttcagaaaagtcaaatattgtatccatacatacatct
ccaaaactatcgcttatacgtttatctttaacgatacctatacctagatggttatttactaacagacattttccagatctattg
actataactcctatagtttccacatcaaccaagtaatgatcatctattgttatataacaataacataactcttttccgtttttatc
agtatgtatatctatatcaacgtcgtcgttgtagtgaatagtagtcattgatctattatatgaaacggatatgtctagaacgg
caattgttttacgtccagttaacactttcgttgatttaaagtctagagtctttgcaaacataatatccttatccgactttatatttc
ctgtagggtggtataattttattttgcctccacatatcggtgtttccaaatatattactagacaatattccatatagttattagtt
aagggtacccaattagaacacgtacgcttattatcatcatttggatcgtatttcataaaagttattgtactatcgatgtcaac
acattctacattttttaatcgtctatatagtatttttctgatattttctataatatcagaattgtcttccatcggaagttgtatactatc
ggaatcagttacatgtttaaataattctctgatgtcattccttatacaatcaaattcattattaaacagtttaatagtctgtaga
cctttatcgtcgtaaatatccattgtcttattagttacgcttatttttatgtgtttttacgttgctttattatattttataagaatgattgtt
tgacgaatcacgagaactattaagacacattattaggtatatattataaaaaagtttttgattacgatgttataagaggaa
agaggacacattaacatcatacatcaattaactacattcttataacatcgtaatcaaaagaattgcaattttgatgtataa
caactgtcaatgggttatggaattgtatattacatattatacggtatgttggtaacgacaaataccgatcggtaattgtctg
ccggtgtaatagaattatatatatctatctattacaccggccttgtatacataataataagttgtggtagtatgatctccatat
ttataatttaggactttgtattcagtttttttggaatcataaaaaataaaaaaaagttttactaatttaaaattatttacatttttttc
actgtttagtcgcggatatggaattcgatcctgccaaaatcaatacatcatctatagatcatgtaacaatattacaataca
tagatgaaccaaatgatataagactaacagtatgcattatcacaaaaataaatccacatttggctaatcaatttcgggct
tggaaaaaacgtatcgccggaagggactatatgactaacttatctagagatacaggaatacaacaatcaaaacttac
tgaaactgtcaaaaaaatagaaacatatatggtctatatatacactacaatttagttattaattggataaccgatgtgatta
tcaatcaatattaagaaggttggtaaattggtacatagctaataatacctatacacccaataatacaacaaccatttctg
agttggatatcatcaaaatactggataaatacgaggacgtgtatagagtaagtaaagaaaaagaatgtgaaatttgct
atgaagttgtttactcaaaacgatagatactttggtttattggattcgtgtaatcatatattttgcataacatgcatcaatatat
ggcatagaacacgaagagaaaccggtgcgtcggataattgtcctatatgtcgtacccgttttagaaacataacaatga
gcaagttaactaataaataaaaagtttaatttgttgacgacgtatgtcgttattttttctcgtataaaagattaatttgattcta
atataatctttagtattggataaatatcaattcaaattaattccattagattatatcataaataaaaatagtagcacgcacta
cttcagccaaatattcttttttgaaacgccatctatcgtagtgaggacacaagtgaacctataatgagcaaatttattagta
tcggttacatgaaggactttacgtagagtggtgattccactatctgtggtacgaacggtttcatcttctttgatgccatcacc
cagatgttctataaacttggtatcctttgccaaccaatacatatagctaaactcaggcatatgttccacacatcctgaaca
atgaaattctccagaagatgttacaatgtctagatttggacatttggtttcaaccgcgttaacatatgagtgaacacaccc
atacatgaaagcgatgagaaataggattttcatcttgccaaaatatcactagaaaaaatttatttatcaattttaaaggtat
aaaaaatacttattgttgctcgaatattttgtatttgatggtatacggaagattagaaatgtaggtattatcatcaactgattc
tatggttttatgtattctatcatgtttcactattgcgttggaaataatatcatatgcttccacatatattttattttgttttaactcata
atactcacgtaattctggattattgacatatctatgaataattttagctccatgatcagtaaatattaatgagaacatagtatt
accacctaccattatttttttcatctcattcaattcttaattgcaaagatctatataatcattatagcgttgacttatggactctg
gaatcttagacgatgtacagtcatctataatcatggcatatttaatacattgttttatagcatagtcgttatctacgatgttag
atatttctctcaatgaatcaatcacacaatctaatgtaggtttatgacataatagcattttcagcagttcaatgtttttagattc
gttgatggcaatggctatacatgtatatccgttatttgatctaatgttgacatctgaaccggattctagcagtaaagatact
agagattgtttattatatctaacagccttgtgaagaagtgtttctcctcgtttgtcaatcatgttaatgtctttaagataaggta
ggcaaatgtttatagtactaagaattgggcaagcataagacatgtcacaaagaccctttttgtatgtataagtgtaaaaa
ttataacattcatagttggatttacataggtgtccaatcgggatctctccatcatcgagataattgatggcatctcccttccttt
tttagtagatatttcatcgtgtaagaatcaatattaatatttctaaagtatccgtgtatagcctctttatttaccacagttccatat
tccactagagggatatcgccgaatgtcatatactcaattagtatatgttggaggacatccgagttcattgttttcaatatca
aaaagatggtttccttatcatttctccatagtggtacaatactacacattattccgtgcggctttccattttccaaaaacaattt
gaccaaatctacatctttattgtatctataatcactatttagataatcagccataattactcgagtgcaacatgttagatcgt
ctatatatgaataagccgtgttatctattcctttcattaacaatttaacgatgtctatatctatatgagatgacttaatataatat
tgaagagctgtacaatagtttttatctataaaagacggcttgattccgtgattaattagacatttaacaacttccggacgca
catatgctctcgtatccgactctgaatacagatgagagatgatatacagatgcaatacggtaccgcaatttcgtagttga
taatcatcatacgcgtatcagtactcgtcctcataaagaacactgcagccattttctatgaacaaatcaataatttcagga
acaggatcatctgtcattacataattttctataactgaacgatggttttcacatttaacactcaagtcaaatccatgttctacc
aacacctttatcaagtcaacgtctacatttttggatttcatatagctgaatatattaaagtcatttatgttgctaaatccagtgg
cttctagtagagccatcgctatatcctttaactttaacatgtctactatttgtgtattcttctaatggggtagctgtctccaattttt
gcgtaatggattagtgccactgtctagtagtagtttgacgacctcgacattattacaatgctcattaaaaaggtatgcgtgt
aaagcattattcttgaattggttcctggtatcattaggatctctgtctctcaacatctgtttaagttcatcgagagccacctcct
cattttccaaatagtcaaacattttgactgaatgagctactgtgaactctatacacccacacaactaatgtcattaaatatc
atgtcaaaaacttgtacaattattaataaaaataatttagtgtttaaattttaccagttccagattttacacctccgttaaccc
cactttttacaccactggacgatcctcctccccacattccaccgccaccagatgtataagttttagatcctttattactacca
tcatgtccatggataaagacactccacatgccgccactaccccctttagaagacatattaataagacttaaggacaag
tttaacaataaaattaatcacgagtaccctactaccaacctacactattatatgattatagtttctatttttacagtaccttaa
ctaaagtctctagtcacaagagcaatactaccaacctacactattatatgattatagtttctatttttataggaacgcgtac
gagaaaatcaaatgtctaatttctaacggtagtgttgataaacgattatcgtcaatggatacctcctctatcatgtcgtctat
tttcttactttgttctattaacttattagcattatatattatttgattataaaacttatattgcttattagcccaatctgtaaatatcgg
attattaacatatcgtttctttgtaggtttatttaacatgtacatcactgtaagcatgtccgtaccatttattttaatttgacgcata
tccgcaatttctttttcgcagtcggttataaattctatatatgatggatacatgctacatgtgtacttataatcgactaatatga
agtacttgatacatattttcagtaacgatttattattaccacctatgaataagtacctgtgatcgtctaggtaatcaactgtttt
cttaatacattcgatggttggtaatttactcagaataatttccaatatcttaatatataattctgctatttctgggatatatttatct
gccagtataacacaaatagtaatacatgtaaacccatattttgttattatattaatgtctgcgccattatctattaaccattct
actaggctgacactatgcgacttaatacaatgataaagtatactacatccatgtttatatcatcaatatacggcttacaaa
gttttagtatcgataacacatccaactcacgcatagagaaggtagggaataatggcataatatttattaggttatcatcat
tgtcattatctacaactaagtttccattttttaaaatatactcgacaactttaggatctctattgccaaatttttgaaaatatttatt
tatatgcttaaatctatataatgtagctccttcatcaatcatacatttaataacattgatgtatactgtatgataagatacatat
tctaacaatagatcttgtatagaatctgtatatcttttaagaattgtggatattattacgtaaactattacacaattctaaaata
taaaacgtatcacggtcgaataatagttgatcaactatataattatcgattttgtgatttttcttcctaaactgtttacgtaaat
agttagatagaatattcattagttcatgaccactatagttactatcgaataacgcgtcaaatatttcccgtttaatatcgcatt
tgtcaagataataatagagtgtggtatgttcacgataagtataataacgcatctctttttcgtgtgaaattaaatagtttatta
cgtccaaagatgtagcataaccatcttgtgacctagtaataatataataatagagaactgttttacccattctatcatcata
atcagtggtgtagtcgtaatcgtaattgtctaattcatcatcccaattataatattcaccagcacgtctaatctgttctattttg
atcttgtatccatactgtatgttgctacatgtaggtattcctttatccaataatagtttaaacacatctacattgggatttgatgtt
gtagcgtatttttctacaatattaataccatttttgatactatttatttctatacctttcgaaattagtaatttcaataagtctatatc
gatgttatcagaacatagatattcgagtatatcaaaatcattgatatttttatagtcgactgacgacaataacaaaatcac
aacatcgtttttgatattattatttttcttggtaacgtatgcctttaatggagtttcaccatcatactcatataatggatttgcacc
actttctatcaatgattgtgcactgctggcatcgatgttaaatgttttacaactatcatagagtatcttatcgttaaccatgatt
ggttgttgatgctatcgcattttttggtttctttcatttcagttatgtatggatttagcacgtttgggaagcatgagctcatatgatt
tcagtactgtagtgtcagtactattagtttcaataagatcaatctctagatctatagaatcaaaacacgataggtcagaa
gataatgaatatctgtaggcttcttgttgtactgtaacttctcgttttgttagatgtttgcatcgtgctttaacatcaatggtaca
aattttatcctcgctttgtgtatcatattcgtccctactataaaattgtatattcagattatcatgagatgtgtatacgctaacgg
tatcaataaacggagcacaccatttagtcataaccgtaatccaaaaatttttaaagtatatcttaacgaaagaagttgtg
tcattgtctacggtgtatggtactagatcctcataagtgtatatatctagagtaatgtttaatttatcaaatggttgataatatg
gatcctcatgacaatttccgaagatggaaatgagatatagacatgcaataaatctaattgcggacatggttactccttaa
aaaaatacgaataatcaccttggctatttagtaagtgtcatttaacactatactcatattaatccatggactcataatctcta
tacgggattaacggatgttctatatacggggatgagtagttttcttctttaactttatactttttactaatcatatttagactgatg
tatgggtaatagtgtttaaagagttcgttctcatcatcagaataaatcaatatctctgtttttttgttatacagatgtattacagc
ctcatatattacgtaatagaacgtgtcatctaccttattaactttcaccgcatagttgtttgcaaatacggttaatcctttgacc
tcgtcgatttccgaccaatctgggcgtataatgaatctaaactttaatttcttgtaatcattcgaaataatttttagtttgcatcc
gtagttatcccctttatgtaactgtaaatttctcaacgcgatatctccattaataatgatgtcgaattcgtgctgtatacccat
actgaatggatgaacgaataccgacggcgttaatagtaatttactttttcatctttacatattgggtactagttttactatcat
aagtttataaattccacaagctactatggaataagccaaccatcttagtataacacacatgtcttaaagtttattaattaatt
acatgttgttttatatatcgctacgaatttaaacagagaaatcagtttaggaaaaaaaaatatctatctacatcatcacgt
ctctgtattctacgatagagtgctactttaagatgagacatatccgtgtcatcaaaaatatactccattaaaatgattattcc
ggcagcgaacttgatattggatatatcacaacctttgttaatatctacgacaatagacagcagtcccatggttccataaa
cagtgagtttatctttctttgaagagatattttgtagagatcttataaaactgtcgaatgacatcgcatttatatctttagctaa
atcgtatatgttaccatcgtaatatctaaccgcgtctatcttaaacgtttccatcgctttaaagacgtttccgatagatggtct
catttcatcagtcatactgagccaacaaatataatcgtgtataacatctttgatagaatcagactctaaagaaaacgaat
cggctttattatacgcattcatgataaacttaatgaaaaatgtttttcgttgtttaagttggatgaatagtatgtcttaataattg
ttattatttcattaattaatatttagtaacgagtacactctataaaaacgagaatgacataactagttatcaaagtgtctagg
acgcgtaattttcatatggtatagatcctgtaagcattgtctgtattctggagctattttctctatatctaatttctgaacgttcac
caatgtctctagccactttggcactaatagcgatcattcgcttagcgtcttctatattattaactggttgattcaatctatctag
caatggaccgtcggacagcgtcattctcatgttcttaatcaatgtacatacatcgccgtcatctaccaattcatccaacaa
cataagctttttaaaatcatcattataataggtttgatcgttgtcatttctccaaagaatatatctaataagtagagtcctcat
gattagttaacaactattttttatgttaaatcaattagtacaccgctatgtttaatacttattcatattttagtttttaggattgaga
atcaatacaaaaattaatgcatcattaattttagaaatacttagtttccacgtagtcaatgaaacatttgaactcatcgtac
aggacgttctcgtacaggacgtaactataaaccggtttatatttgttcaagatagatacaaatccgataactttttttacga
attctacgggatccactttaaaagtgtcataccgggttctttttatttttttaaacagattaatggtgtgatgttgattaggtctttt
acgaatttgatatagaatagcgtttacatattctccataatggtcaatcgccatttgttcgtatgtcataaattctttaattatat
gacactgtgtattatttagttcatccttgttcatcattaggaatctatccaatatggcaattatactagaactataggtgcgttg
tatacacatattgatgtgtctgtttatacaatccatgctactaccttcgggtaaaattgtagcatcatataccatttctagtact
ttaggttcattgttatccattgcagaggacgtcatgatcgcatcctaaaaaaatatattatttttatgttattttgttaaaaataa
tcatcgaatacgaatcatccagtccactgaatagcaaaatctttactattttggtatcttccaatgtggctgcctgatgtaat
ggaaattcattctctagaagatttttcaatgctccagcgttcaacaacgtacatactagacgcacgttattatcagctattg
cataatacaaggcactatgtccatggacatccgccttaaatgcatctttgctagagagaaagcttttcagctgcttagact
tccaagtattaattcgtgacagatccatgtctgaaacgagacgctaattagtgtataatttttgtcatattgcaccagaatta
ataatatctctaatagatctgattagtagatacatggctatcgcaaaacaacatatacacatttaataaaaataatatttat
taagaaaattcagatttcacgtacccatcaatataaataaaataatgattccttacaccgtacccatattaaggagattct
accttacccataaacaatataaatccagtaatatcatgtctgatgatgaacacaaatggtgtattaaattccagtttttcag
gagatgatctcgccgtagctaccataatagtagatgcctctgctacagttccttgttcgtcgacatctatctttgcattctga
aacattttataaatatataatgggtccctagtcatatgtttaaacgacgcattatctggattaaacatactaggagccatca
tttcggctatcgacttaatatccctcttattttcgatagaaaatttagggagtttaagattgtacactttattccctaattgaaac
gaccaatagtctaattttgcagccgtaatagaatctgtgaaatgggtcatattatcacctattgccaggtacatactaatat
tagcatccttatacggaaggcgtaccatgtcatattctttgtcatcgattgtgattgtatttccttgcaatttagtaactacgttc
atcatgggaaccgttttcgtaccgtacttattagtaaaactagcattgcgtgttttagtgatatcaaacggatattgccatat
acctttaaaatatatagtattaatgattgcccatagagtattattgtcgagcatattagaatctactacattagacataccg
gatctacgttctactatagaattaattttattaaccgcatctcgtctaaagtttaatctatataggccgaatctatgatattgttg
ataatacgacggtttaatacacacagtattatctacgaaactttgataagttagatcagtgtacgtatatttagatgttttca
gcttagctaatcctgatattaattctgtaaatgctggacccagatctctttttctcaaatccatagtcttcaataattctattcta
gtattacctgatgcaggcaatagcgacataaacatagaaaacgaataaccaaacggtgagaagacaatattatcat
cttgaatatttttatacgctactataccggcattggtaaatccttgtagacgataggtagacgctgaacacgttaacgata
gtatcaataacgcaatcatgattttatggtattaataattaaccttatttttatgttcggtataaaaattattgatgtctacacat
ccttttgtaattgacatctatatatccttttgtataatcaactctaatcactttaacttttacagttttccctaccagtttatccctat
attcaacatatctatccatatgcatcttaacactctctgccaagatagcttcagagtgaggatagtcaaaaagataaata
tatagagcataatcattctcgtatactctgccctttattacatcgcccgcattgggcaacgaataacaaaatgcaagcat
cttgttaacgggctcgtaaattgggataaaaattatgtttttatatctattttattcaagagaatattcaggaatttctttttccgg
ttgtatctcatcgcagtatatatcatttgtacattgtttcatattttttaatagtttacaccttttagtaggactagtatcgtacaatt
catagctgtattttgaattccaatcacgcataaaaatatcttctaattgttgacgaagacctaatccatcatccggtgtaat
attaatagatgctccacatgtatccgtaaagtaatttcctgtccaatttgaggtacctatataggccgttttatcggttaccat
atatttggcatggtttaccctagaatacggaatgggaggatcagcatctggtacaataaatagctttacttctatatttatgt
ttttagattttagcatagcgatagatcttaaaaagtttctcatgataaacgaagatcgttgccagcaactaatcaatagctt
aacggatacttgtctgtctatagcggatcttcttaattcatcttctatataaggccaaaacaaaattttacccgccttcgaat
aaataatagggataaagttcataacagatacataaacgaatttactcgcatttctaatacatgacaataaagcggttaa
atcattggttctttccatagtacatagttgttgcggtgcagaagcaataaatacagagtgtggaacaccacttacgttaat
actaagaggatgatctgtattataatacgacggataaaagtttttccaattatatggtagattgttaactccaagatacca
gtatacctcaaaaatttgagtgagatccgctgccaagttcctattattgaagatcgcaatacccaattctttgacctgagtt
agtgatctccaatccatgttagcgcttcctaaataaatatgtgtattatcagatatccaaaattttgtatgaagaactcctcc
taggatatttgtaatatctatgtatcgtacttcaactccggccatttgtagtctttcaacatcctttaatggtttgttagatttattg
acggctactctaactcgtactcctcttttgggtaattgtacaatctcgtttaatattatcgtgccgaaattcgtacccacttcat
ccgataaactccaataaaaagatgatatatctagtgtttttgtggtattggatagaatttccctccacatgttaaatgtaga
caaatatactttatcaaattgcatacctataggaatagtctctgtaatcactgcgattgtattatccggattcattttatttgtta
aaagaataatcctatatcacttcactctattaaaaatccaagtttctatttctttcatgactgattttttaacttcatccgtttcctt
atgaagatgatgtttggcaccttcataaatttttatttctctattacaatttgcatgttgcatgaaataatatgcacctaaaac
atcgctaatctcattgtttgttccctggagtatgagagtcggggtgttaatcttggaaattatttttctaaccttgttggtagcctt
caagacctgactagcaaatccagccttaattttttcatgattgattaatgggtcgtattggtatttataaactttatccatatct
ctagatactgattctggacatagctttccgactggcgcatttggtgtgatggttcccataagtttggcagctagcagattca
gtcttgaaacagcatctgcattaactagaggagacattagaatcattgctgtaaacaagtttggattatcgtaagaggct
agtatagaaattgttgctcccatggaatgacccaataagtagatttaatagttaccacgtgctgtaccaaagtcatcaat
catcattttttcaccattacttcttccatgtccaatatgatcatgtgagaatactaaaattcctaacgatgatatgttttcagct
agttcgtcataacgtccagaatgtttaccagctccatgacttatgaatactaatgccttaggatatgtaatcattgtccaga
ttgaacatacagtttgcactcatgattcacgttatataactatcaatattaacagttcgtttgatgatcatattatttttatgtttta
ttgataattgtaaaaacatacaattaaatcaatatagaggaaggagacggctactgtcttttgtgagatagtcatggcga
ctaaattagattatgaggatgctgttttttactttgtggatgatgataaaatatgtagtcgcgactccatcatcgatctaatag
atgaatatattacgtggagaaatcatgttatagtgtttaacaaagatattaccagttgtggaagactgtacaaggaattg
atgaagttcgatgatgtcgctatacggtactatggtattgataaaattaatgagattgtcgaagctatgagcgaaggag
accactacatcaattttacaaaagtccatgatcaggaaagtttattcgctaccataggaatatgtgctaaaatcactgaa
cattggggatacaaaaagatttcagaatctagattccaatcattgggaaacattacagatctgatgaccgacgataat
ataaacatcttgatactttttctagaaaaaaaattgaattgatgatataggggtcttcataacgcataattattacgttagca
ttctatatccgtgttaaaaaaaattatcctatcatgtatttgagagttttatatgtagcaaacatgatagctgtgatgccaata
agctttagatattcacgcgtgctagtgttagggatggtattatctggtggtgaaatgtccgttatataatctacaaaacaat
catcgcatatagtatgcgatagtagagtaaacatttttatagtttttactggattcatacatcgtctacccaattcggttatga
atgaaattgtcgccaatcttacacccaaccccttgttatccattagtatagtattaacttcgttatttatgtcataaactgtaa
atgattttgtagatgccatatcatacatgatattcatgtccctattataatcattactaactttatcacaatatatgttgataata
tctatatatgatctagtctttgtgggcaactgtctatacaagtcgtctaaacgttgtttactcatatagtatcgaacagccatc
attacatggtcccgttccgttgatagataatcgagtatgttagtggacttgtcaaatctatataccatattttctggaagtgg
atatacatagtcgtgatcaacattattgctagcctcatcttctatatcctgtactataccatctacataatctacgatattatta
cacataaacatcgacaacatactattgtttattatctaagtcctgttgatccaaacccttgatctcctctatttgtactatctag
agattgtacttcttccagttctggataatatatacgttgatagattagctgagctattctatctccagtatttacattaaacgta
cattttccattattaataagaatgactcctatgtttcccctataatcttcgtctattacaccacctcctatatcaatgccttttagt
gacagaccagacctaggagctattctaccatagcaaatcttaggcatggacatactaatatctgtcttaattaactgtctt
tctcctggagggatagtataatcgtaagcgctatacaaatcatatccggcagcacccggcgattgcctagtaggagat
ttagctctgttagtttccttaacaaatctaactggtgagttaatattcatgttgaacataaaactaatattttatttcaaaattatt
taccatcccatatattccatgaataagtgtgatgattgtacacttctatagtatctatatacgattcacgataaaatcctcct
atcaatagcagtttattatccactatgatcaattctggattatccctcggataaataggatcatctatcagagtccatgtatt
gctggattcacaataaaattccgcatttctaccaaccaagaataaccttctaccgaacactaacgcgcatgatttataat
gaggataataagtggatggtccaaactgccactgatcatgattgggtagcaaatattctgtagttgtatcagtttcagaat
gtcctcccattacgtatataacattgtttatggatgccactgctggattacatctaggtttcagaagactcggcatattaacc
caagcagcatccccgtggaaccaacgctcaacagatgtgggatttggtagacctcctactacgtataatttattgttagc
gggtatcccgctagcatacagtctggggctattcatcggaggaattggaatccaattgtttgatatataatttacagctata
gcattgttatgtatttcattgttcatccatccaccgatgagatatactacttctccaacatgagtacttgtacacatatggaat
atatctataatttgatccatgttcataggatactctatgaatggatacttgtatgatttgcgtggttgtttatcacaatgaaatat
tttggtacagtctagtatccattttacattatttatacctctgggagaaagataatttgacctgattacatttttgataaggagt
agcagatttcctaatttatttcttcgctttatataccacttaatgacaaaatcctcatctggaacatttagttcatcgctttctag
aataagtttcatagatagataatcaaaattgtctatgatgtcatcttccagttccaaaaagtgtttggcaataaagtttttagt
atgacataagagattggatagtccgtattctatacccatcatgtaacactcgacacaatattcctttctaaaatctcgtag
gataaagtttatacaagtgtagatgataaattctacagaggttaatatagaagcacgtaataaattgacgacgttatga
ctatctatatatacctttccagtatatgagtaaataactatagaagttagactgtgaatgtcaaggtctagacaaaccctc
gtaactggatctttatttttcgtgtatttttgacgtaaatgtgtgcgaaagtaaggagataactttttcaatatcgtagaattga
ctattatattgcctcctatggcatcaataattgttttgaatttcttagtcatagacaatgctaatatattcttacagtacacagta
ttgacaaatatcggcatttatgtttctttaaaagtcaacatctaaagaaaaatgattatcttcttgagacataactcccattttt
tggtattcacccacacgtttttcgaaaaaattagttttaccttctaatgatatattttccatgaaatcaaacggattggtaaca
ttataaatttttttaaatcccaattcagaaatcaatctatccgcgacgaattctatatatgttttcatcatttcacaattcattcct
ataagtttaactggaagagccgcagtaagaaattcttgttcaatggataccgcatctgttataatagatctaacggtttctt
cactcggtggatgcaataaatgtttaaacatcaaacatgcgaagtcgcagtgtagaccctcgtctctactaatcaattcg
ttggaaaacgtgagtccgggcattaggccacgctttttaagccaaaatatggaagcgaatgatccggaaaagaaga
ttccttctactgcagcaaaggcaataagtctctctccataaccggcgctgtcatgtatccacttttgagcccaatcggcctt
cttttttacacaaggcatcgtttctatggcattaaagagatagtttttttcattactatctttaacataagtatcgatcaaaaga
ctatacatttccgaatgaatgttttcaatggccatctgaaatccgtagaaacatctagcctcggtaatctgtacttctgtaca
aaatcgttccgccaaattttcattcactattccgtcactggctgcaaaaaacgccaatacatgttttataaaatatttttcgtc
tggtgttagtttattccaatcattgatatctttagatatatctacttcttccactgtccaaaatgatgcctctgcctttttatacatg
ttccagatgtcatgatattggattgggaaaataacaaatctatttggatttggtgcaaggatgggttccataactaaattaa
caataacaataaattttttttcagttatctatatgcctgtacttggatcttttgtacatcgatatcgccgcaatcactacaataa
ttacaagtattattgatagcattgttattagtactatcataattaaattatcgttattatcattttgtaattgtgacatcatactaga
taaatcgtttgcgagattgttgtgggaagcgggcatggaggatgaattatcgttattattatttaaagcctcccattcggatt
cacaaatatggcgcgcgttcaacattttatggaaacagataacaagaaaactcgtcatcgttcaaatttttaacgatagt
aaaccgattaaacgtcgagctaatttctaacgctagcgactctgttggatatgggtttccagatatatatcttttcagttccc
ctacgtatctataatcatctgtaggaaatggaagatatttccatttatctactgttcctaatatcatatgtggtggtgtagtag
aaccattaagcgcgaaagatgttatttcgcatcgtattttaacttcgcaataatttctggttagataacgcactctaccagt
caagtcaatgatattagcctttacagatatattcatagtagtcgtaacgatgactccatcttttagatgcgatactcctttgta
tgtaccagaatcttcgtacctcaaactcgatatatttaaacaagttaatgagatattaacgcgttttatgaatgatgatatat
aaccagaagttttatcctcggtggctagcgctataaccttatcattataataccaactagtgtgattaatatgtgacacgtt
agtgtgggtacaaatatgtacattatcgtctacgtcgtattcgatacatccgcatacagccaacaaatataaaatgaca
aatactctaacgccgttcgtacccatcttgatgcggtttaataaatgttttgatttcaatttattgtaaaaaaagattcggtttt
atactgttcgatattctcattgcttatattttcatctatcatctccacacagtcaaatccgtggttagcatgcacctcatcaacc
ggtaaaagactatcggactcttctatcattataactctagaatatttaatttggtcattattaatcaagtcaattatcttattttta
acaaacgtgagtattttactcattttttataaaaacttttagaaatatacagactctatcgtgtgtctatatcttctttttatatcca
atgtatttatgtctgatttttcttcatttatcatatataatggtccaaattctacacgtgcttcggattcatccagatcattaaggtt
cttataattgtaacatccttctcttccctcttctacatcttccttcttattcttattcttagcgtcacagaatctaccacagcagga
tcccatgacgagcgtcatattaaactaatccattttcaattataatatacgattagtaatgaccattaaaataaaaatattct
tcataaccggcaagaaagtgaaaagttcacattgaaactatgtcagtagtatacatcatgaaatgatgatatatatata
ctctattttggtggaggattatatgatataattcgtggataatcattcttaagacacatttcttcattcgtaaatcttttcacgtta
aatgagtgtccatattttgcaatttcttcatatgatggcggtgtacgtggacgaggctgctcctgttcttgttgtagtcgccga
ctgtcgtgtctgcgtttagatccctccattatcgcgattgcgtagatggagtactattttataccttgtaattaaatttttttattaa
ttaaacgtataaaaacgttccgtatctgtatttaagagccagatttcgtctaatagaacaaatagctacagtaaaaataa
ctagaataattgctacacccactagaaaccacggatcgtaatacggcaatcggttttcgataataggtggaacgtatat
tttatttaaggacttaacaattgtctgtaaaccacaatttgcttccgcggatcctgtattaactatctgtaaaagcatatgttg
accgggcggagccgaacattctccgatatccaatttctgtatatctataatattattaacctccgcatacgcattacagttc
ttttctagcttggataccgcactaggtacatcgtctagatctattcctatttcctcagcgatagctcttctatccttttccggaa
gcaatgaaatcacttcaataaatgattcaaccatgagtgtgaaactaagtcgagaattactcatgcatttgttagttattc
ggagcgcgcaatttttaaactgtcctataacctctcctatatgaatagcacaagtgacattagtagggatagaatgttga
gctaatttttgtaaataactatctataaaaagattatacaaagttttaaactctttagtttccgccatttatccagtctgagaa
aatgtctctcataataaatttttccaagaaactaattgggtgaagaatggaaacctttaatctatatttatcacagtctgtttt
ggtacacatgatgaattcttccaatgccgtactaaattcgatatctttttcgatttctggatatgtttttaataaagtatgaaca
aagaaatggaaatcgtaataccagttatgtttaactttgaaattgttttttattttcttgttaatgattccagccacttgggaaa
agtcaaagtcgtttaatgccgatttaatacgttcattaaaaacaaactttttatcctttagatgaattattattggttcattgga
atcaaaaagtaagatattatcgggtttaagatctgcgtgtaaaaagttgtcgcagcatggtagttcgtaaattttaatgtat
aacagagccatctgtaaaaagataaactttatgtattgtaccaaagatttaaatcctaatttgatagctagctcggtatct
actttatctgccgaatacagtgctaggggaaaaattataatatttcctctttcgtattcgtagttagttctcttttcatgttcgaa
aaagtgaaacatgcggttaaaatagtttataacattaatattactgttaataactgccggataaaagtgggatagtaattt
cacgaatttgatactgtcctttctctcgttaaacgcctttaaaaaaactttagaagaatatctcaatgatagttcctgaccat
ccatagtttgtatcaataatagcaacatatgaagaacacgtttatacagagtatgtaaaaatgttaatttatagtttaatcc
catggcccacgcacacacgattaattttttttcatctccctttagattgttgtatagaaatttgggtactgtgaactccgccgt
agtttccatgggactatataattttgtggcctcgaatacaaattttactacatagttatctatcttaaagactataccatatcct
cctgtagatatgtgataaaaatcgtcgtttataggataaaatcgtttatccttttgttggaaaaaggatgaattaatgtaatc
attctcttctatctttagtagtgtttccttattaaaattcttaaaataatttaacaatctaactgatggagcccaattttggtgtaa
atctaattgggacattatattgttaaaatacaaacagtctcctaatataacagtatctgataatctatggggagacatcca
ttgatattcaggggatgaatcattggcaacacccatttattgtacaaaaagccccaatttacaaacgaaagtccaggttt
gatagagacaaactattaactattttgtctctgtttttaatttctttggtaatgaaattattcacaatatcagtatcttctttatcta
ccagagattttactaacttgataaccttggctgtctcattcaatagggtagtaatatttgtatgtgtgatattgatatcttttaga
agtgattctttgatggtgccagcatacgaattacaataatgcagaaactcggttaacatgcaggaattatagtaagcca
attccaattgttgcctgtgttgtattagagtgtcaatatgagcaatggtgtccttgcgtttctctgatagaatgcgagcagcg
attttggcgttatcatttgacgatatttctggaatgacgaatcctgtttctactaactttttggtaggacaaagtgaaacaatc
aagaagatagcttctcctcctatttgtggaagaaattgaactcctctagatgatctccttgacagatattggaccgaatta
cagaagtacctggaatgtaaagccctgaaaccccctcattttttaagcagattgttgccgtaaatcctgcactatgccca
agatagagagctcctttggtgaatccatctctatgtttcagtttaaccaagaaacagtcagctggtctaaaatttccatctc
tatctaatacagcatctaacttgatgtcaggaactatgaccggttatgttatatgtaacattgagtaaatccttaagttcata
atcatcactgtcatcagttatgtacgatccaaacaatgtttctactggcatagtggatacgaagatgctatccatcagaat
gtttccctgattagtattttctatatagctattcttctttaaacgattttccaaatcagtaactatgttcatttttttaggagtaggac
gcctagccagtatggaagaggattttctagatcctctcttcaacatctttgatctcaatggaatgcaaaaccccatagtgt
aacaaccaacgataaaaataatattgtttttcactttttataattttaccatctgactcatggattcattaatatctttataaga
gctactaacgtataattctttataactgaactgagatatatacaccggatctatggtttccataattgagtaaatgaatgctc
ggcaataactaatggcaaatgtataaaacaacgaaattatactagagttgttaaagttaatattttctatgagctgttcca
ataaattatttgttgtaactgcgttcaagtcataaatcatcttgatactatccagtaaaccgtttttaagttctggaatattatta
tcccattgtaaagcccctaattcgactatcgaatatcctgctctgatagcagtttcaatatcgacggacgtcaatactgta
ataaaggtggtagtattgtcatcatcgtgataaactactggaatatggtcgttagtaggtacggtaactttacacaacgc
gatatataactttccttttgtaccatttttaacgtagttgggacgtcctgcagggtattgttttgaagaaatgatatcgagaac
agatttgatacgatatttgttggattcctgattatttactataatataatctagacagatagatgattcgataaatagagaag
gtatatcgttggtaggataatacatccccattccagtattctcggatactctattaatgacactagttaagaacatgtcttct
attctagaaaacgaaaacatcctacatggactcattaaaacttctaacgctcctgattgtgtctcgaatgcctcgtacaa
ggatttcaaggatgccatagattctttgaccaacgatttagaattgcgtttagcatctgatttttttattaaatcgaatggtcg
gctctctggtttgctaccccaatgataacaatagtcttgtaaagataaaccgcaagaaaatttatacgcatccatccaaa
taaccctagcaccatcggatgatattaatgtattattatagattttccatccacagttattgggccagtatactgttagcaac
ggtatatcgaatagattactcatgtaacctactagaatgatagttcgtgtactagtcataatatctttaatccaatctaaga
aatttaaaattagattttttacactgttaaagttaacaaaggtattacccggatacgtggatatcatatatggtattggtccat
tatcagtaatagctccataaactgatacggcgatggtttttatatgtgtttgatctaacgaggaagaaattcgcgcccaca
attcatctctagatatgtatttaatatcaaacggtaacacatcaatttcgggacgcgtatatgtttctaaatttttaatccaaat
ataatgatgacctatatgccctattatcatactgtcaactatagtacacctagagaacttacgatacatctgtttcctataat
cgttaaattttacaaatctataacatgctaaaccttttgacgacaaccattcattaatttctgatatggaatctgtattctcaat
accgtatcgttctaaagccagtgctatatctccctgttcgtgagaacgctttcgtataatatcgatcaacggataatctgaa
gtttttggagaataatatgactcatgatctatttcgtccataaacaatctagacataggaattggaggcgatgatcttaattt
tgtgcaatgagtcgtcaatcctataacttctaatattgtaatattcatcatcgacataacactatctatgttatcatcgtatatt
agtataccatgaccttcttcatttcgtgccaaaatgatatacagtcttaaatagttacgcaatatctcaatagtttcataattg
ttagctgttttcatcaaggtttgtatcctgtttaacatgatggcgttctatacgtttctattttttaaatttttaacgatttactgtggc
tagatacccaatctctctcaaatatttttttagcctcgcttacaagctgtttatctatactattaaaactgacgaatccgtgattt
tggtaatgggttccgtcgaaatttgccgaagtgatatgaacatattcgtcgtcgactatcaacaattttgtattattctgaat
agtgaaaaccttcacagatagatcattttgaacacacaacgcgtctagacttctggcggttgccatagaatatacgtcg
ttcttatcccaattaccaactagaagtctgatcttaactcctctattaatggctgcttctataatggagttgtaaatgtcgggc
caatagtagctattaccgtcgacacgtgtagtgggaactatggccaaatgttcaatatctatactagtcttagccgacttg
agtttatcaataactacatcagtgtctagatctctagaatatcccaataggtgttccggagaatcagtaaagaacactcc
acctataggattcttaatatgatacgcagtgctaactggcagacaacaagccgcagagcataaattcaaccatgaatt
ttttgcgctattaaaggctttaaaagtatcaaatcttctacgaagatctgtggccagcgggggataatcagaatatacac
ctaacgttttaatcgtatgtatagatcctccagtaaatgacgcgtttcctacataacatctttcattatctgacacccaaaaa
caaccgagtagtagtcccacattattttttttatctatattaacggttataaaatttatatccgggcagtgactttgtagctctc
ccagatttcttttccctcgttcatctagcaaaactattattttaatccctttttcagatgcctcttttagtttatcaaaaataagcg
ctcccctagtcgtactcagaggattacaacaaaaagatgctatgtatatatatttcttagctagagtgataatttcgttaaa
acattcaaatgttgttaaatgatcggatctaaaatccatattttctggtagtgtttctaccagcctacattttgctcccgcagg
taccggtgcaaatggccacatttagttaacataaaaacttatacatcctgttctatcaacgattctagaatatcatcggct
atatcgctaaaattttcatcaaagtcgacatcacaacctaactcagtcaatatattaagaagttccatgatgtcatcttcgt
ctatttctatatccgtatccattgtagattgttgaccgattatcgagtttaaatcattactaatactcaatccttcagaatacaa
tctgtgtttcattgtaaatttataggcggtgtatttaagttggtagattttcaattatgtatcaatatagcaacagtagttcttgct
cctccttgattctagcatcctcttcattattttcttctacgtacataaacatgtccaatacgttagacaacacaccgacgatg
gcggccgccacagacacgaatatgactaaaccgatgaccatttaaaaacccctctctagctttcacttaaactgtatcg
attattcttttagaacatgtataatataaaaacattattctatttcgaatttaggcttccaaaaatttttcatccgtaaaccgat
aataatatatatagacttgttaatagtcggaataaatagattaatgcttaaactatcatcatctccacgattagagataca
atatttacattttttttgctgtttcgaaactttatcaatacacgttaatacaaacccaggaaggagatattgaaactgaggct
gttgaaaatgaaacggtgaatacaataattcagataatgtaaaatcatgattccgtattctgatgatattagaactgctaa
tggatgtcgatggtatgtatctaggagtatctattttaacaaagcatcgatttgctaatatacaattatcattttgattaattgtt
attttattcatattcttaaaaggtttcatatttatcaattcttctacattaaaaatttccatttttaatttatgtagccccgcaatact
cctcattacgtttcattttttgtctataatatccattttgttcatctcggtacatagattatccaattgagaagcgcatttagtagtt
ttgtacattttaagtttattgacgaatcgtcgaaaactagttatagttaacattttattatttgataccctgatattaatacccct
gccgttactattatttataactgatgtaatccacgtaacattggaattaactatcgatagtaatgcatcgacgcttccaaaa
ttgtctattataaactcaccgataatttttttattacatgttttcatattcattaggattattaaatctttaatcttactacgattgtat
gcgttgatattgcaagacgtcattctaaaagacggaggatctccatcaaatgccagacaatcacgtacaaagtacat
ggaaataggttttgttctattgcgcatcatagatttatatagaacacccgtagaaatactaatttgttttactctataaaatac
taatgcatctatttcatcgttttgtataacgtctttccaagtgtcaaattccaaatttttttcattgatagtaccaaattcttctatct
ctttaactacttgcatagataggtaattacagtgatgcctacatgccgttttttgaaactgaatagatgcgtctagaagcga
tgctacgctagtcacaatcaccactttcatatttagaatatatatatgtaaaaatatagtagaatttcattttgttttttttctatg
ctataaatgaattctcattttgcatctgctcatactccgttttatatcaataccaaagaaggaagatatctggttctaaaagc
cgttaaagtatgcgatgttagaactgtagaatgcgaaggaagtaaagcttcctgcgtactcaaagtagataaaccctc
atcacccgcgtgtgagagaagaccttcgtccccttccagatgcgagagaatgaataacccaggaaaacaagttccg
tttatgaggacggacatgctacaaaatatgttcgcggctaatcgcgataatgtagcttctagacttttgtcctaaaataca
attatatccttttcgatattaataaatccgtgtcgtccaggttttttatctctttcagtatgtgaatagataggtattttatctctattc
atcatcgaatttaagagatccgataaacattgtttgtattctccagatgtcagcatctgatacaacaatatatgtgcacata
aacctctggcacttatttcatgtaccttccccttatcactaaggagaatagtatttgagaaatatgtatacatgatattatcat
gaattagatatacagaatttgtaacactctcgaaatcacacgatgtgtcggcgttaagatctaatatatcactcgataac
acattttcatctagatacactagacattttttaaagctaaaatagtctttagtagtaacagtaactatgcgattattttcatcg
atgatacatttcatcggcatattattacgcttaccatcaaagactataccatgtgtatatctaacgtattctagcatggttgc
catacgcgcattaaacttttcaggatctttggatagatcttccaatctatctatttgagaaaacatttttatcatgttcaatagtt
gaaacgtcggatccactatatagatattatctataaagattttaggaactacgttcatggtatcctggcgaatattaaaac
tatcaatgatatgattatcgttttcatcttttatcaccatatagtttctaagatatgggattttacttaatataatattatttcccgta
ataaattttattagaaatgccaaatctataagaaaagtcctcgaattagtttgaagaatatctatatcgccgtaccgtatat
ttggattaattagatatagagaatatgatccgtaacatatacaacttttattatggcgtctaagatattcttccatcaacttatt
aacatttttgactagggaagatacattatgacgtcccattacttttgccttgtctattactgcgacgttcatagaatttagcat
atctcttgccaattcttccattgatgttacattataagaaattttagatgaaattacatttggagctttaatagtaagaactcct
aatatgtccgtgtatgtggtcactaatacagattgtagttctataatcgtaaataatttacctatattatatgtttgagtctgttta
gaaaagtagctaagtatacgatcttttatttctgatgcagatgtatcaacatcggaaaaaaatctttttttattcttttttactaa
agatacaaatatgtctttgttaaaaacagttattttctgaatatttctagcttgtaattttaacatatgatattcgttcacactag
gtactctgcctaaataggtttctataatctttaatgtaatattaggaaaagtattctgatcaggattcctattcattttgaggatt
taaaactctgattattgtctaatatggtctcaacacaaactttttcacagagcgatagagtttttgataactcgtttttcttaag
aaatataaaactactgtctccagagctcgctctatcttttattttatttaattcgatacaaactcctgatactggttcagaaag
taattcattaattttcagtcctttatagaagatatttaatatagataatacaaaatcttcagtttttgatatcgatctgattgatcc
tagaactagatatattaataacgtgctcattaggcagtttatggcagcttgataattagatatagtatattccagttcatattt
attagataccgcattgcccagattttgatattctatgaattcctctgaaaataaatccaaaataactagacattctattttttgt
ggattagtgtactctcttccctctatcatgttcactactggtgtccacgatgataaatatctagagggaatataatatagtcc
ataggatgccaatctagcaatgtcgaataactgtaattttattcttcgctcttcattatgaattgattcttgaggtataaaccta
acacaaattatattattagacttttcgtatgtaatgtctttcatgttataagtttttaatcctggaatagaatctattttaatgagg
cttttaaacgcagagttctccaacgagtcaaagcataatactctgttggttttcttatatacgatgttacgattttcttctttgaa
tggaataggtttttgaattagtttataattacaacataatagataaggaagtgtgcaaatagtacgcggaaaaaacata
atagctcccctgttttcatccatggttttaagtaaatgatcactggcttctttagtcaatggatattcgaacattaaccgtttca
tcatcattggacagaatccatatttcttaatgtaaagagtgatcaaatcattgtgtttattgtaccatcttgttgtaaatgtgtat
tcggttatcggatctgctcctttttctattaaagtatcgatgtcgatctcgtctaagaattcaactatatcgacatatttcatttgt
atacacataaccattactaacgtagaatgtataggaagagatgtaacgggaacagggtttgttgattcgcaaactattc
taatacataattcttctgttaatacgtcttgcacgtaatctattatagatgccaagatatctatataattattttgtaagatgatg
ttaactatgtgatctatataagtagtgtaataattcatgtatttcgatatatgttccaactctgtctttgtgatgtctagtttcgtaa
tatctatagcatcctcaaaaaatatattcgcatatattcccaagtcttcagttctatcttctaaaaaatcttcaacgtatgga
atataataatctattttacctcttctgatatcattaatgatatagtttttgacactatcttctgtcaattgattcttattcactatatct
aagaaacggatagcgtccctaggacgaactactgccattaatatctctattatagcttctggacataattcatctattata
ccagaattaatgggaactattccgtatctatctaacatagttttaagaaagtcagaatctaagacctgatgttcatatattg
gttcatacatgaaatgatctctattgatgatagtgactatttcattctctgaaaattggtaactcattctatatatgctttccttgtt
gatgaaggatagaatatactcaatagaatttgtaccaacaaactgttctcttatgaatcgtatatcatcatctgaaataat
catgtaaggcatacatttaacaattagagacttgtctcctgttatcaatatactattcttgtgataatttatgtgtgaggcaaa
tttgtccacgttctttaattttgttatagtagatatcaaatccaatggagctacagttcttggcttaaacagatatagtttttctg
gaacgaattctacaacattattataaaggactttgggtagataagtgggatgaaatcctattttaattaatgcgatagcctt
gtcctcgtgcagatatccaaacgcttttgtgatagtatggcattcattgtctagaaacgctctacgaatatctgtgacagat
atcatctttagagaatatactagtcgcgttaatagtactacaatttgtattttttaatctatctcaataaaaaaattaatatgtat
gattcaatgtataactaaactactaactgttattgataactagaatcagaatctaatgatgacgtaaccaagaagtttatc
tactgccaatttagctgcattatttttagcatctcgtttagattttccatctgccttatcgaatactcttccgtcgatatctacaca
ggcataaaatgtaggagagttactaggccccactgattcaatacgaaaagaccaatctctcttagttatttggcagtact
cattaataatggtgacagggttagcatctttccaatcaataatttttttagccggaataacatcatcaaaagacttatgatc
ctctctcattgatttttcgcgggatacatcatctattatggcgtcagccataacatcagcatccggcttatccgcctccgttgt
cataaaccaacgaggaggaatatcgtcggagctgtacaccatagcactacgttgaagatcgtacagagctttattaa
cttctcgcttctccatattaagttgtctagttagttgtgcagcagtagctccttcgattccaatgtttttaatagccgcacacac
aatctctgcgtcagaacgctcgtcaatatagatcttagacatttttagagagaactaacacaaccagcaataaaacta
atttattttatcatttttttattcatcatcctctggtggttcgtcgtttctatcgaatgtggatctgattaacccgtcatctataggtg
atgctggttctggagattctggaggagatggattattatctggaagaatctctgttatttccttgttttcatgtatcgattgcgtt
gtaacattaagattgcgaaatgctctaaatttgggaggcttaaagtgttgtttgcaatctctacacgcatgtctaactagtg
gaggttcgtcagcggctctagtttgaatcatcatcggcgtagtattcctacttttacagttaggacacggtgtattgtatttct
cgtcgagaacgttaaaataatcgttgtaactcacatcctttattttatctatattgtattctactcctttcttaatgcattttatacc
gaataagagatagcgaaggaattctttttcggtgccgctagtacccttaatcatatcacatagtgttttatattccaaatttgt
ggcaatagacggtttatttctatacgatagtttgtttctggaatcctttgagtattctataccaatattattctttgattcgaattta
gtttcttcgatattagattttgtattacctatattcttgatgtagtactttgatgatttttccatggcccattctattaagtcttccaag
ttggcatcatccacatattgtgatagtaattctcggatatcagtagcggctaccgccattgatgtttgttcattggatgagta
actactaatgtatacattttccatttataacacttatgtattaactttgttcatttatattttttcattattatgttgatattaacaaaa
gtgaatatatatgttaataattgtattgtggttatacggctacaatttcataatgagtggaagtcagtgtccgatgatcaatg
acgatagctttactctgaaaagaaagtatcaaatcgatagtgcggagtcaacaataaaaatggataagaagaggat
aaagtttcagaatagagccaaaatggtaaaagaaataaatcagacaataagagcagcacaaactcattacgaga
cattgaaactaggatacataaaatttaagagaatgattaggactactactctagaagatatagcaccatctattccaaa
taatcagaaaacttataaactattctcggacatttcagccatcggcaaagcatcacagaatccgagtaagatggtatat
gctctgctgctttacatgtttcccaatttgtttggagatgatcatagattcattcgttatagaatgcatccaatgagtaaaatc
aaacacaagatcttctctcctttcaaacttaatcttattagaatattagtggaagaaagattctataataatgaatgcagat
ctaataaatggagaataattggaacacaagttgataaaatgttgatagctgaatctgataaatatacaatagatgcaa
ggtataacctaaaacccatgtatagaatcaagggaaaatctgaagaagataccctctttatcaaacagatggtagaa
caatgtgtgacatcccaggaattggtggaaaaagtgttgaagatactgtttagagatttgttcaagagtggagaataca
aagcgtacagatacgatgatgatgtagaaaatggatttattggattggatacactaaaattaaacattgttcatgatata
gttgaaccatgtatgcctgttcgtaggccagtggctaagatactgtgtaaagaaatggtaaataaatactttgagaatcc
gctacatattattggtaaaaatcttcaagagtgcattgactttgttagtgaataggcatttcatctttctccaatactaattcaa
attgttaaattaataatggatagtataaatagttattagtgataaaatagtaaaaataattattagaataagagtgtagtat
catagataactctcttctataaaaatggattttattcgtagaaagtatcttatatacacagtagaaaataatatagatttttta
aaggatgatacattaagtaaagtaaacaattttaccctcaatcatgtactagctctcaagtatctagttagcaattttcctc
aacacgttattactaaggatgtattagctaataccaatttttttgttttcatacatatggtacgatgttgtaaagtgtacgaag
cggttttacgacacgcatttgatgcacccacgttgtacgttaaagcattgactaagaattatttatcgtttagtaacgcaat
acaatcgtacaaggaaaccgtgcataaactaacacaagatgaaaaatttttagaggttgccgaatacatggacgaa
ttaggagaacttataggcgtaaattatgacttagttcttaatccattatttcacggaggggaacccatcaaagatatgga
aatcatttttttaaaactgtttaagaaaacagacttcaaagttgttaaaaaattaagtgttataagattacttatttgggctta
cctaagcaagaaagatacaggcatagagtttgcggataatgatagacaagatatatacactctatttcaacaaactg
gtagaatagtccatagcaatctaacagaaacgtttagagattatatctttcccggagataagactagctattgggtgtgg
ttaaacgaaagtatagctaatgatgcggatattgttcttaatagacacgccattaccatgtatgataaaattcttagttatat
atactctgagataaaacaaggacgcgttaataaaaacatgcttaagttagtttatatctttgagcctgaaaaagatatca
gagaacttctgctagaaatcatatatgatattcctggagatatcctatctattattgatgcaaaaaacgacgattggaaa
aaatattttattagtttttataaagctaattttattaacggtaatacatttattagtgatagaacgtttaacgaggacttattcag
agttgttgttcaaatagatcccgaatatttcgataatgaacgaattatgtctttattctctacgagtgctgcggacattaaac
gatttgatgagttagatattaataacagttatatatctaatataatttatgaggtgaacgatatcacattagatacaatggat
gatatgaagaagtgtcaaatctttaacgaggatacgtcgtattatgttaaggaatacaatacatacctgtttttgcacgag
tcggatcccatggtcatagagaacggaatactaaagaaactgtcatctataaaatccaagagtagacggctgaactt
gtttagcaaaaacattttaaaatattatttagacggacaattggctcgtctaggtcttgtgttagatgattataaaggagact
tgttagttaaaatgataaaccatcttaagtctgtggaggatgtatccgcattcgttcgattttctacagataaaaaccctagt
attcttccatcgctaatcaaaactattttagctagttataatatttccatcatcgtcttatttcaaaggtttttgagagataatcta
tatcatgtagaagaattcttggataaaagcatccatctaaccaagacggataagaaatatatacttcaattgataagac
acggtagatcatagaacagaccaaatatattattaataatttgtatatacatagatataattatcacacatttttgataaatg
ggaactgctgcaacaattcagactcccaccaaattaatgaataaagaaaatgcagaaatgattttggaaaaaattgtt
gatcatatagttatgtatattagtgacgaatcaagtgattcagaaaataatcctgaatatattgattttcgtaacagatacg
aagactatagatctctcattataaaaagtgatcacgagtttgtaaagctatgtaaaaatcatgcagagaaaagttctcc
agaaacgcaacaaatgattatcaaacacatatacgaacaatatcttattccagtatctgaagtactattaaaacctata
atgtccatgggtgacataattacatataacggatgtaaagacaatgaatggatgctagaacaactctctaccctaaact
ttaacaatctccgcacatggaactcatgtagcataggcaatgtaacgcgtctgttttatacattttttagttatctgatgaaa
gataaactaaatatataagtataatcccattctaatactttaacctgatgtattacctgcatcttattagaatattaacctaac
taaaagacataacatagttgataaaaagcggtaggatataaatattatggctgccaccgttccgcgttttgacgacgtg
tacaaaaatgcacaaagaagaattctagatcaagaaacattttttagtagaggtctaagtagaccgttaatgaaaaac
acatatctatttgataattacgcgtatggatggataccagaaactgcaatttggagtagtagatacgcaaacttagatgc
aagtgactattatcccatttcgttgggattacttaaaaagttcgagtttctcatgtctctatataaaggtcctattcccgtatat
gaagaaaaagtaaatactgaattcattgctaatggatcgttctctggtagatacgtatcatatcttcgaaagttttctgccct
tccaacaaacgagtttattagttttttgttactgacttccattccaatctataatatcttgttctggtttaaaaatactcagtttgat
attactaaacacacattattcagatacgtctatacagataatgccaaacacctggcgttggctaggtatatgcatcaaa
caggagactataagcctttgtttagtcgtctcaaagagaattatatatttaccggtcccgttccaataagtatcaaagatat
agatcaccctaatcttagtagagcaagaagtccatccgattatgagacattagctaatattagtactatattgtactttacc
aagtatgatccggtattaatgtttttattgttttacgtacctgggtattcaattactacaaaaattactccagccgtagaatatc
taatggataaactgaatctaacaaagagcgacgtacaactgttgtaaattattttatgcttcgtaaaatgtaggttttgaac
caaacattctttcaaagaatgagatgcataaaactttattatccaatagattgactatttcggacgtcaatcgtttaaagta
aacttcgtaaaatattctttgatcactgccgagtttaaaacttctatcgataattgtttcatatgttttaatatttacaagttttttg
gtccatggtacattagccggacaaatatatgcaaaataatatcgttctccaagttctatagtttctggattatttttattatattc
agtaaccaaatacatattagggttatctgcggatttataatttgagtgatgcattcgactcaacataaataattctagagg
agacgatctactatcaaattcggatcgtaaatctgtttctaaagaacggagaatatctatacatacctgattagaattcat
ccgtccttcagacaacatctcagacagtctggtcttgtatgtcttaatcatattcttatgaaacttggaaacatctcttctagtt
tcactagtacctttattaattctctcaggtacagattttgaattcgacgatgctgagtatttcatcgttgtatatttcttcttcgatt
gcataatcagattcttatataccgcctcaaactctattttaaaattattaaacaatactctattattaatcagtcgttctaactc
tttcgctatttctatagacttatcgacatcttgactgtctatctctgtaaacacggagtcggtatctccatacacgctacgaa
aacgaaatctgtaatctataggcaacgatgttttcacaatcggattaatatctctatcgtccatataaaatggattacttaat
ggattggcaaaccgtaacataccgttagataactctgctccatttagtaccgattctagatacaagatcattctacgtcct
atggatgtgcaactcttagccgaagcgtatgagtatagagcactatttctaaatcccatcagaccatatactgagttggc
tactatcttgtacgtatattgcatggaatcatagatggccttttcagttgaactggtagcctgttttagcatctttttatatctggc
tctctctgccaaaaatgttcttaatagtctaggaatggttccttctatcgatctatcgaaaattgctatttcagagatgaggtt
cggtagtctaggttcacaatgaaccgtaatatatctaggaggtggatatttctgaagcaagagctgattatttatttcttctt
ccaatctattggtactaacaacgacaccgactaatgtttccggagatagatttccaaagatacacacattaggataca
gactgttataatcaaagattaatacattattactaaacattttttgttttggagcaaataccttaccgccttcataaggaaact
tttgttttgtttctgatctaactaagatagttttagtttccaacaatagctttaacagtggacccttgatgactgtactcgctctat
attcgaataccatggattgaggaagcacatatgttgacgcacccgcgtctgtttttgtttctactccataatactcccacaa
atactgacacaaacaagcatcatgaatacagtatctagccatatctaaagctatgtttagattataatccttatacatctg
agctaaatcaacgtcatcctttccgaaagataatttatatgtatcattaggtaaagtaggacatgatagtacgactttaaa
tccattttcccaaatatctttacgaattactttacatataatatcctcatcaacagtcacataattacctgtggttaaaaccttt
gcaaatgcagcggctttgcctttcgcgtccgtagtatcgtcaccgatgaacgtcatttctctaactcctctatttaatacttta
cccatgcaactgaacgcgttcttggatatagaatccaatttgtacgaatccaatttttcagatttttgaatgaatgaatatag
atcgaaaaatatagttccattattgttattaacgtgaaacgtagtattggccatgccgcctactcccttatgactagactga
tttctctcataaatacagagatgtacagcttcctttttgtccggagatctaaagataatcttctctcctgttaataactctagac
gattagtaatatatctcagatcaaagttatgtccgttaaaggtaacgacgtagtcgaacgttagttccaacaattgtttag
ctattcgtaacaaaactatttcagaacatagaactagttctcgttcgtaatccatttccattagtgactgtatcctcaaacat
cctctatcgacggcttcttgtatttcctgttccgttaacatctcttcattaatgagcgtaaacaataatcgtttaccacttaaat
cgatataacagtaacttgtatgcgagattgggttaataaatacagaaggaaacttcttatcgaagtgacactctatatct
agaaataagtacgatcttgggatatcgaatctaggtatttttttagcgaaacagttacgtggatcgtcacaatgataacat
ccattgttaatctttgtcaaatattgctcgtccaacgagtaacatccgtctggagatatcccgttagaaatataaaaccaa
ctaatattgagaaattcatccatggtggcattttgtatgctgcgtttctttggctcttctatcaaccacatatctgcgacggag
cattttctatctttaatatctagattataacttattgtctcgtcaatgtctatagttctcatctttcccaacggcctcgcattaaatg
gaggaggagacaatgactgatatatttcgtccgtaactacgtaataaaagtaatgaggaaatcgtataaatacggtct
cgccatttcgacatctggatttcagatataaaaatctgttttcaccgtgactttcaaaccaattaatgcaccgaacatccat
ttatagaatttagaaatatattttcatttaaatgaatcccaaacattggggaagagccgtatggaccattatttttatagtact
ttcgcaagcgggtttagacggcaacatagaagcgtgtaaacgaaaactatatactatagtcagcactcttccatgtcct
gcatgtagacggcacgcgactatcgctatagaggacaataatgtcatgtctagcgatgatctgaattatatttattatttttt
catcagattatttaacaatttggcatctgatcccaaatacgcgatcgatgtgacaaaggttaaccctttataaacttaacc
cattataaaacttatgattagtcacgactgaaataaccgcgtgattattttttggtataattctacacggcatggtttctgtaa
ctatgaattcaacccccgttacattagtgaaatctttaacaaacagcaagggttcgtcaaagacataaaactcattgttt
acaatcgaaatagaccccctatcacacttaaaataaaaaatatccttatcctttaccaccaaataaaattctgattggtc
aatgtgaatgtattcacttaacagttccacaaatttatttattaactccgaggcacatacatcgtcggtattttttatggcaaa
ctttactcttccagcatccgtttctaaaaaaatattaacgagttccatttatatcatccaatattattgaaatgacgttgatgg
acagatgatacaaataagaaggtacggtacctttgtccaccatctcctccaattcatgctctattttgtcattaactttaatgt
atgaaaacagtacgccacatgcttccatgacagtgtgtaacactttggatacaaaatgtttgacattagtataattgtcca
agactgtcaatctataatagatagtagctataatatattctatgatggtattgaagaagatgacaatcttggcatattgatc
atttaacacagacatggtatcaacagatagcttgaatgaaagagaatcagtaattggaataagcgtcttctcgataga
gtgtccgtataccaacatgtctgatattttgatgtattccattaaattatttagttttttctttttattctcgttaaacagcatttctgtc
aacggaccccaacatcgttgaccgattaagttttgattgatttttccgtgtaaggcgtatctagtcagatcgtatagcctat
ccaataatccatcatctgtgcgtagatcacatcgtacactttttaattctctatagaagagcgacagacagcaatttctttat
tctctacagatgtaagatacttgaagacattcctatgatgatgcagaattttggataacacggtattgatggtatctgttac
cataattcctttgatggctgatagtgtcagagcacaagatttccaatctttgttttgatatctatatcagacagcatggtgcgt
ctgacaacacaaggattaagacggaaagatgaaatgattctctcaacatcttcaatggataccttgctattttttctggca
ttatctatatgtgcgagaatatcctctagagaatcagtatcctttttgatgatagtggatctcaatgacatgggacgtctaa
accttcttattctatcaccagattgcatggtgatttgtcttctttcttttatcataatgtaatctctaaattcatcggcaaattgtct
atatctaaaatcataatatgagatgtttacctctacaaatatctgttcgtccaatgttagagtatctacatcagttttgtattcc
aaattaaacatggcaacggatttaattttatattcctctattaagtcctcgtcgataataacagaatgtagataatcatttaa
tccatcgtacatggttggaagatgcttgttgacaaaatctttaattgtcttgatgaaggtgggactatatctaacatcttgatt
aataaaatttataacattgtccataggatactttgtaactagttttatacacatctcttcatcggtaagtttagacagaatatc
gtgaacaggtggtatattatattcatcagatatacgaagaacaatgtccaaatctatattgtttaatatattatatagatgta
gcgtagctcctacaggaatatctttaactaagtcaatgatttcatcaaccgttagatctattttaaagttaatcatataggca
ttgatttttaaaaggtatgtagccttgactacattctcattaattaaccattccaagtcactgtgtgtaagaagattatattcta
tcataagcttgactacatttggtcccgataccattaaagaattcttatgatataaggaaacagattttaggtactcatctact
ctacaagaattttggagagccttaacgatatcagtgacgtttattatttcaggaggaaagaatctaacattgagaatatc
ggaattaatagcttccagatacagtgattttggcaatagtccgtgtaatccataatccagtaacacgagctggtgcttgct
agacaccttttcaatgtttaatttttttgaaataagctttgataaagccttcctcgcaaattccggatacatgaacatgtcgg
cgacatgattaagtattgttttttcattattttctcaacaagttctcaataccccaatagatgatagaatatcacccaatgcgt
ccatgttgtctatttccaacaggtcgctatatccaccaatagaagtttttccaaaaaagattctaggaacagttctaccac
cagtaatttgttcaaaatagtcacgcaattcattttcgggtttaaattctttaatatcgacaatttcatacgctcctcttttgaaa
ctaaacttatttagaatatccagtgcatttctacaaaaaggacatgtatacttgacaaaaattgtcactttgttattggccaa
cctttgttgtacaaattcctcggccattttaatatttaagtgatataaaactatctcgacttatttaactctttagtcgagatatat
ggacgcagatagctatatgatagccaactacagaaggcaaacgctataaaaaacataattacgacgagcatatttat
aaatatttttattcagcattacttgatatagtaatattaggcacagtcaaacattcaaccactctcgatacattaactctctc
attttctttaacaaattctacaatatcttcgtaaaaagattcttgaaactttttagaatatctatcgactctagatgaaatagc
gttcgtcaacatactatgttttgtatacataaaggcgcccattttaacagtttctagtgacaaaatgctagcgatcctagga
tcctttagaatcacatagattgacgattcgtctctcttagtaactctagtaaaataatcatacaatctagtacgcgaaataa
tattatccttgacttgaggagatctaaacaatctagttttgagaacatcgataagttcatcgggaatgacatacatactatc
tttaatagaactcttttcatccagttgaatggattcgtccttaaccaactgattaatgagatcttctattttatcattttccagatg
atatgtatgtccattaaagttaaattgtgtagcgcttctttttagtctagcagccaatactttaacatcactaatatcgatatac
aaaggagatgatttatctatggtattaagaattcgtttttcgacatctgtcaaaaccaattcctttttgcctgtatcatccagttt
tccatcctttgtaaagaaattattttctactagactattaataagactgataaggattcctccataattgcacaatccaaact
ttttaacaaaactagactttacaagatctacaggaatgcgtaattcaggtttcttagcttgtgattttttcttttgtggacattttc
ttgtgaccaactcatctaccatttcattgattttagcagtgaaataagctttcaatgcacgggcactgatactattgaaaac
gagttgatcttcaaattccgccatttaagttcaccaaacaacttttaaatacaaatatatcaatagtagtagaataagaac
tataaaaaaaataataattaaccaataccaaccccaacaaccggtattattagttgatgtgactgttttctcatcacttag
aacagatttaacaatttctataaagtctgtcaaatcatcttccggagaccccataaatacaccaaatatagcggcgtac
aacttatccatttatacattgaatattggcttttctttatcgctatcttcatcatattcatcatcaatatcaacaagtcccagatta
cgagccagatcttcttctacattttcagtcattgatacacgttcactatctccagagagtccgataacgttagccaccactt
ctctatcaatgattagtttcttgagtgcgaatgtaatttttgtttccgttccggatctatagaagacgataggtgtgataattgc
cttggccaattgtctttctcttttactgagtgattctagttcaccttctatagatctgagaatggatgattctccagtcgaaaca
tattctaccatggatccgtttaatttgttgatgaagatggattcatccttaaatgttttctctgtaatagtttccaccgaaagact
atgcaaagaatttggaatgcgttccttgtgcttaatgtttccatagacggcttctagaagttgatacaacataggactagc
cgcggtaacttttatttttagaaagtatccatcgcttctatcttgtttagatttatttttataaagtttagtctctccttccaacataa
taaaagtggaagtcatttgactagataaactatcagtaagttttatagagatagacgaacaattagcgtattgagaagc
atttagtgtaacgtattcgatacattttgcattagatttactaatcgattttgcatactctataacacccgcacaagtctgtag
agaatcgctagatgcagtaggtcttggtgaagtttcaactctcttcttgattaccttactcatgattaaacctaaataattgta
ctttgtaatataatgatatatattttcactttatctcatttgagaataaaaatgtttttgtttaaccactgcatgatgtacagatttc
ggaatcgcaaaccaccagtggttttattttatccttgtccaatgtgaattgaatgggagcggatgcgggtttcgtacgtag
atagtacattcccgtttttagaccgagactccatccgtaaaaatgcatactcgttagtttggaataactcggatctgctata
tggatattcatagattgactttgatcgatgaaggctcccctgtctgcagccatttttatgatcgtcttttgtggaatttcccaaa
tagttttataaactcgcttaatatcttctggaaggtttgtattctgaatggatccaccatctgccataatcctattcttgatctca
tcattccataattttctctcggttaaaactctaaggagatgcggattaactacttgaaattctccagacaatactctccgagt
gtaaatattactggtatacggttccaccgactcattatttcccaaaatttgagcagttgatgcagtcggcataggtgccac
caataaactatttctaagaccgtatgttctgattttatcttttagaggttcccaattccaaagatccgacggtacaacattcc
aaagatcatattgtagaataccgttactggcgtacgatcctacatatgtatcgtatggtccttccttctcagctagttcacaa
ctcgcctctaatgcaccgtaataaatggtttcgaagatcttcttatttagatcttgtgcttccaggctatcaaatggataattt
aagagaataaacgcgtccgctaatccttgaacaccaataccgataggtctatgtctcttattagagatttcagcttctgg
aataggataataattaatatctataattttattgagatttctgacaattactttgaccacatccttcagtttgagaaaatcaaa
tcgcccatctattacaaacatgttcaaggcaacagatgccagattacaaacggctacctcattagcatccgcatattgt
attatctcagtgcaaagattactacacttgatagttcctaaattttgttgattactctttttgttacacgcatccttataaagaat
gaatggagtaccagtttcaatctgagattctataatcgctttccagacgactcgagcctttattatagatttgtatctcctttct
ctttcgtatagtgtatacaatcgttcgaactcgtctccccaaacattgtccaatccaggacattcatccggacacatcaac
gaccactctccgtcatccttcactcgtttcataaagagatcaggaatccaaagagctataaatagatctctggttctatgtt
cctcgtttcctgtattctttttaagatcgaggaacgccataatatcagaatgccacggttccaagtatatggccataactcc
aggccgtttgtttcctccctgatctatgtatctagcggtgttattataaactctcaacattggaataataccgtttgatatacc
attggtaccggagatatagcttccactggcacgaatattactaattgatagacctattccccctgccattttagagattaat
gcgcatcgttttaacgtgtcatagataccctctatgctatcatcgatcatgttaagtagaaaacagctagacatttggtga
cgactagttcccgcattaaataaggtaggagaagcgtgcgtaaaccatttttcagaaagtagattgtacgtctcaatag
ctgagtctatatcccattgatgaattcctactgcgacacgcattaacatgtgctgaggtctttcaacgatcttgttgtttattttc
aacaagtaggatttttccaaagttttaaaaccaaaatagttgtatgaaaagtctcgttcgtaaataataaccgagttgagt
ttatccttatatttgttaactatatccatggtgatacttgaaataatcggagaatgtttcccatttttaggattaacatagttgaa
taaatcctccatcacttcactaaatagtttttttgtttccttgtgtagatttgatacggctattctggcggctagaatggcataat
ccggatgttgtgtagtacaagtggctgctatttcggctgccagagtgtccaattctaccgttgttactccattatatattccttg
aataaccttcatagctattttaataggatctatatgatccgtgtttaagccataacataattttctaatacgagacgtgatttt
atcaaacatgacattttccttgtatccatttcgtttaatgacaaacatttttgttggtgtaataaaaaaaattatttaacttttcat
taatagggatttgacgtatgtagcgtacaaaattatcgttcctggtatatagataaagagtcctatatatttgaaaatcgtta
cggctcgattaaactttaatgattgcatagtgaatatatcattaggatttaactccttgactatcatggcggcgccagaaat
taccatcaaaagcattaatacagttatgccgatcgcagttagaacggttatagcatccaccatttatatctaaaaattag
atcaaagaatatgtgacaaagtcctagttgtatactgagaattgacgaaacaatgtttcttacatattttttttttattagtaac
cgacttaatagtaggaactggaaaactagacttgattattctataagtatagatacccttccaaataatattctctttgata
aaagttccagaaaatgtagaattttttaaaaagttatcttttgctattaccaagattgtgtttagacgcttattattaatatgag
tgatgaaatccacaccgcctctagatatcgcttttatttccacattagatggtaaatccaatagtgaaactatctttttagga
atgtatggactcgcgtttagaggagtgaacgtcttaggcgtcggaaaggggccggcctttttttctagacactaaataaa
tagtaagattaaattaattataaaattatgtatataatattaattataaaattatgtatatgatttactaactttagttagataaa
ttaataatacataaattttagtatattaatattataaattaataatacataaattttagtatattaatattatattttaaatatttattt
agtgtctagaaaaaaaggcctagcaggcc

Any of the sequences of the sMVA fragments F1, F2, and F3 may contain one or more alterations or variations. For example, the sequence of sMVA fragment F1 (deposited at NCBI under Accession No. MW023923, www.ncbi.nlm.nih.gov/nuccore/MW023923.1/) contains 1 nucleotide alteration in a non-coding determining region downstream of open reading frame 021 (SEQ ID NO: 12):

ggcctagcaggccggcctttttttctagacactaaataaatagtaagattaaattaattataaaattat
gtatataatattaattataaaattatgtatatgatttactaactttagttagataaattaataatacataaattttagtatattaat
attataaattaataatacataaattttagtatattaatattatattttaaatatttatttagtgtctagaaaaaaatgtgtgacca
acgaccgtaggaaactctagagggtaagaaaaatcaatcgctttatagagaccatcagaaagaggtttaatatttttgt
gagaccatcgaaggagaaagagataaaacttttttacgactccatcagaaagaggtttaatatttttgtgagaccatcg
aagagagaaagagataaaacttttttacgactccatcagaaagaggtttaatatttttgtgagaccatcgaagagaga
aagagataaaacttttttacgactccatcagaaagaggtttaatatttttgtgagaccatcgaaggagaaagagataaa
acttttttacgactccatcagaaagaggtttaatatttttgtgagaccatcgaaggagaaagagataaaacttttttacga
ctccatcagaaagaggtttaatatttttgtgagaccatcgaaggagaaagagataaaacttttttacgactccatcaga
aagaggtttaatatttttgtgagaccatcgaagagagaaagagataaaacttttttacgactccatcagaaagaggttta
atatttttgtgagaccatcgaaggagaaagagataaaacttttttacgactccatcagaaagaggtttaatatttttgtgag
accatcgaagagagaaagagaataaaaatattttagtgacaccatcagaaagaggtttaatatttttgtgagaccatc
gaagagagaaagagataaaacttttttacgactccatcagaaagaggtttaatatttttgtgagaccatcgaaggaga
aagagataaaacttttttacgactccatcagaaagaggtttaatatttttgtgagaccatcgaagagagaaagagataa
aacttttttacgactccatcagaaagaggtttaatatttttgtgagaccatcgaagagagaaagagataaaacttttttac
gactccatcagaaagaccatcgaagagagaaagagaaagagatagttagtctagatatttttcttagtacaaaagtc
aatgttttaaaatatatggacaagaatttgtctgtataaaaacttgtgtgaaattttgtaccaaagaaaaaatgtgagcag
tatcccctacatggattttactagatcatttatataccaaaaaatattatacgatctacgttttattatatgattttaacgtgtaa
attataaacattattttatgatatacaattgtctggtaacctagatgggcataggggatgagtatatgttgttggacgttattg
tttaagaaatagttgatgcatcagaaagaggtttaatatttttgtgagaccatcgaagagagaaagagataaaactttttt
acgactccatcagaaagaggtttaatatttttgtgagaccatcgaagagagaaagagataaaacttttttatgactccat
tgaagagagaatgagaataaaaatattttagtgacaccatcagaaagaggtttaatattttttatgagaccatcaaaga
gagaaagagaataaaaatattttatgactccattgaagagagaaagagaaaatgagaataaaaatattttagtgaca
ccatcagaaagaggtttaatattttttatgagaccatcaaagagagaaagagaataaaaatatttttgtaaaacttttttta
tgagaccatcaaagagagaaagagaataaaaatatttttgtaaaactttttttatgagaccatcaaagagagaaaga
gaataaaaatatttttgtaaaactttttttatgagaccatcaaagagagaaagagaataaaaatatttttgtaaaacttttttt
atgagaccatcaaagagagaaagagaataaaaatatttttgtaaaactttttttatgagaccatcaaagagagaaag
agaataaaaatatttttgtaaaactttttttatgagaccatcaaagagagaaagagaataaaaatatttttgtaaaacttttt
ttatgagaccatcaaagagagaaagagaataaaaatattttatgactccattgaagagagaaagagaataaaaata
ttttagtgacaccatcagaaagaggtttaatatttttgtgagaccatcgaagagagaaagagaataaaaatattttatga
ctccattgaagagagaaagagaataaaaatattttagtgacaccatcagaaagaggtttaatattttttatgagaccatc
aaagagagaaagagaataaaaatatttttgtaaaactttttttatgagaccatcaaagagagaaagagaataaaaat
atttttgtaaaactttttttatgagaccatcaaagagagaaagagaataaaaatatttttgtaaaactttttttatgagaccat
caaagagagaaagagaataaaaatatttttgtaaaactttttttatgagaccatcaaagagagaaagagaataaaaa
tatttttgtaaaactttttttatgagaccatcaaagagagaaagagaataaaaatattttatgactccattgaagagagaa
tgagaataaaaatattttagtgacaccatcagaaagaggtttaatatttttgtgagaccatcgaagagagaaagagaa
taaaaatattttatgactccattgaagagagaaagagaataaaaatattttagtgacaccatcagaaagaggtttaata
ttttttatgagaccatcaaagagagaaagagaataaaaatatttttgtaaaactttttttatgagaccatcaaagagaga
aagagaataaaaatatttttgtaaaactttttttatgagaccatcaaagagagaaagagaataaaaatatttttgtaaaa
ctttttttatgagaccatcaaagagagaaagagaataaaaatatttttgtaaaactttttttatgagaccatcaaagagag
aaagagaataaaaatatttttgtaaaactttttttatgagaccatcaaagagagaaagagaataaaaatatttttgtaaa
actttttttatgagaccatcaaagagagaaagagaataaaaatatttttgtaaaactttttttatgagaccatcaaagaga
gaaagagaataaaaatattttatgactccattgaagagagaatgagaataaaaatattttagtgacaccatcagaaa
gaggtttaatatttttgtgagaccatcgaagagagaaagagaataaaaatattttatgactccattgaagagagaatga
gaataaaaatattttagtgacaccatcagaaagaggtttaatattttttatgagaccatcaaagagagaaagagaata
aaaatatttttgtaaaactttttttatgagaccatcaaagagagaaagagaataaaaatatttttgtaaaaattataaacat
tattttatgatatacaattgtctggtaacctagatgggcataggggatgttgataagctcgacgagtatatgttgttggacgt
tattgtttaagaaatagttgatgcatcagaaagagaataaaaaatattttagtgagaccatcgaagagagaaagagat
aaaacttttttacgactccatcagaaagaggtttaatatttttgtgagaccatcgaagagagaaagagataaaactttttt
acgactccatcagaaagaggtttaatatttttgtgagaccatcgaaggagaaagagataaaacttttttacgactccatc
agaaagaggtttaatatttttgtgagaccatcaaagagagaaagagaataaaaatatttttgtaaaactttttttatgaga
ccatcaaagagagaaagagaataaaaatatttttgtaaaactttttttatgagaccatcaaagagagaaagagaata
aaaatatttttgtaaaactttttttatgagaccatcaaagagagaaagagaataaaaatatttttgtaaaactttttttatgag
accatcaaagagagaaagagaataaaaatattttatgactccattgaagagagaatgagaataaaaatattttagtg
acaccatcagaaagaggtttaatatttttgtgagaccatcgaagagagaaagagaataaaaatattttatgactccatt
gaagagagaatgagaataaaaatattttagtgacaccatcagaaagaggtttaatattttttatgagaccatcaaaga
gagaaagagaataaaaatatttttgtaaaactttttttatgagaccatcaaagagagaaagagaataaaaatatttttgt
aaaactttttttatgagaccatcaaagagagaaagagaataaaaatatttttgtaaaactttttttatgagaccatcaaag
agagaaagagaataaaaatatttttgtaaaactttttttatgagaccatcaaagagagaaagagaataaaaatattttat
gactccattgaagagagaatgagaataaaaatattttagtgacaccatcagaaagaggtttaatatttttgtgagaccat
cgaagagagaaagagaataaaaatattttatgactccattgaagagagaaagagaataaaaatattttagtgacac
catcagaaagaggtttaatattttttatgagaccatcaaagagagaaagagaataaaaatatttttgtaaaactttttttat
gagaccatcaaagagagaaagagaataaaaatattttatgactccattgaagagagaatgagaataaaaatatttta
gtgacaccatcagaaagaggtttaatatttttgtgagaccatcgaagagagaaagagaataaaaatattttatgactcc
attgaagagagaatgagaataaaaatattttagtgacaccatcagaaagaggtttaatatttttgtgagaccatcgaag
agagaaagagaataaaaatattttatgactccattgaagagagaatgagaataaaaatattttagtgacaccatcag
aaagaggtttaatattttttatgagaccatcaaagagagaaagagaataaaaatatttttgtaaaactttttttatgagacc
atcaaagagagaaagagaataaaaatatttttgtaaaactttttttatgagaccatcaaagagagaaagagaataaa
aatatttttgtaaaactttttttatgagaccatcaaagagagaaagagaataaaaatatttttgtgagaccatcaaagag
agaaagagaataaaaatatttttgtgagaccatcaaagagagaaagagaataaaaatatttttgtgagaccatcaaa
gagagaaagagaataaaaatattttatgactccattgaagagagaaagagaataaaaatattttagtgacaccatca
gaaagaggtttaatatttttgtgagaccatcgaagagagaaagagaataaaaatattttatgactccattgaagagag
aaagagaataaaaatattttagtgacaccatcagaaagaggtttaatattttttatgagaccatcaaagagagaaaga
gaataaaaatattttttatgagaccatcaaagagagaaagagaataaaaatattttatgagaccatcaaagagagaa
agagaataaaaatattttttatgagaccatcaaagagagaaagagaataaaaatattttatgagaccatcaaagaga
gaaagagaataaaaatattttttatgagaccatcaaagagagaaagagaataaaaatatttttgtatgagaccatcag
aaagaggtttaatatttttgtgataccctgaaaggaaataggaatagtgtcataatcgtatcacactattgagacagaaa
aagaagaagtcgcgagaggtaactttttgttttgcaaaccggaatatagtgtccggtacacttttttaattcgtggtgtgcc
tgaatcgttcgattaaccctactcatccaatttcagatgaatagagttatcgattcagacacacgctttgagttttgttgaat
cgatgagtgaagtatcatcggttgcaccttcagatgccgatccgtcgacatacttgacctcaagttcagatgattccttgc
acatgtctccgatacgaacgctaaactctagattcttgacacattttgtatcgacgatcgttgaaccgatgatatcttcgta
actcactttcttatgagagatgttagacccgagtactggatgggtcttgatgtcgctgtctttctcttcttcgctacatctgatgt
cgatagacacctcacagtctttccatcagcggattctgagatggatttaatctgaggacatttggtgaatccaaagttcat
tctcagacctccaccgatgatggagtaataagtggtaggaggatctacatcctcgactgattccacctcgggatctgga
tctgactcggactctgtaatttccgttacggattggcaaatcttatcatcggtcggtgtttggtcttgctttgtgactttgataat
aacatcgattcccatatgatgtttgttttcttcttccgtacacgatgaggatgattgctgaagactggcaggcacatgcatg
ccagtacgatatattgtttcatgattgctattgattgagtactgttctttatgattctacttccttaccgtgcaataaattagaata
tattttctacttttacgagaaattaattattgtatttatgggtgaaaaacttactataaaaagcgggtgggtttggaattagtg
atcagtttatgtatatcgcaactaccgggcatatggctacattacccacatgataagagattgtatcagtttcgtagtcttg
agtattggtattactatatagtatatagatgtcgacgctagagttactgtctccgaatgcggcatgatagtatcattctttgct
ttcgttaactgtttggaggaagaatctttgttattgcatttaatctcgaaattcagagtgcacacctttctcctgtaaagaaac
ctgaagtcgctaccttattaagaagacgggatcgcagtctttatgattcatagtaatagttagttccgacgttgagatgga
ttcgctgagaccggtagtggtcgtccgagtacacgatgtgtcgttaactggatacaggttaatttccacatcgatatagtt
aaaggtatttctgggtacgggttcgcatttatctgcggaagagacggtgtgagaatatgttccgagaccacacggaga
acagatgacgtctccggatactccgtatcctattccacattttgtttgggaaacacatgccttgcatccatgatcgggaga
gcattcacagattctattgtgagtcgtgttacacgatcgcgtcgacattgttgacagaaacgtgaccttcattcttaccgtc
gtccataaatacgttaggtatgtaccacatactgtcgcgaacgatgcgtccatctcataatgatttactttttcataattaaa
gatgtgaaagaaaaccgaacaatatatttttttagtaatgtttatgcgagacatataaaataaactccgtgtttatgatgcc
ggtaaatgtttttatcatcttggacggaatcgattttgtaatatgccatggaaacaggacattatcactccatgataaattat
ttaatggagtcgatcctctcattgttctttgcgtatctcaatctgtggcgtttgcttcgtttaaataatatatcaaacatggaga
cgcctgatatgtaggcattcttcattctattaatgtctgctctatagcgctttagttccttatgacgaccggcgatatcatactt
actttagaaggaaaatcatcatctaggattaaggcgtatctgatacaggcgaataatggttcaggatatagatagcgta
tatctctattaaatgcgtcaatcatagtctctagagtgggatggtaactcagtaataaatcaactagcttctctttggtaact
gcttttctggatggccgtattgattatcgagcgtgacactcgctccatattccaataaccgctttgcaaattgtatattattga
catcgaccgcgtaatatagtagagttatcgatcatatctatatcatccatgtacttgcttagtatatcaaatacatcttcata
acagtgatacccgcaattattaaatctcgataatatcagaccgtacatacatagacggccattgttagatatgtgatttac
agccgcgtgtccatattttccacgataaaccttacgacgtttacatcgacgagattattattaacaaagttgttgtccgtcgt
cttatccaacatgcattgaatgataggtatacttaccatatcgccgtaatgtaagtagtttatcagtatggcttgtacgatgg
attcatcctgttgtctaaatctctttagaatgttatcgatgatgtagtggttatattctctggaatcgtacgaagtaatactacg
cattacgtcgacaagagtatgacgtctctcaataagaagattaacgatttccatgtctacattatatggggttactctaaat
cgcttgtttagataatacgcctctaatatagggctgacgtcgtatactctacacgtgtccacatcctttattaataatctctat
atctatggttgagcaagaccagtagtattggatggaaacattgttatcgatcaaacatttaattacatccttggatagaga
ttctctatgagacgatatatagtaatgaagagagttcttacacatatcactgttgtacatacaggtacgaaatacgtaacc
ggtgctgtaacattctgatttaagaagccatagcaatacttctggtctcggattaggcgtcgttacgtatatatccaccaat
ccgagaccattgattgcataattcgtattcttggacggacgtatccgtttatccacaattaggtattttagcagacgtaagt
cgaaatcatttatattcgacttgagttcgttagaggaattcgaatagctggatatcagtagatgcacaatctgagattttac
gtatctatgcttactgtatgctcctagcggagttaatccttcgttgtttctacaaagtctctcgactccgcgagagagtaaca
gtcgaacaatcttaatgtctgtatcgcatttattggagacgtaacaatgtagcgcattgtttcctcgtctatctatatgttttgat
aagttgtgacacgtttcaatttctagttttatttttttgtacgtcacatcttcatccagtagacgacatagaatacatgtgcaat
ccatagctattctggtgctaattattcctcataagatgataaaaagtgtagtgagagagcatgaaggagatttagtattta
gcagtgcggatatgatccaagagggtgagatagtcgttctcgttcagaatctttcgcagcataagtagtatgtcgatata
cttatcgttgaagactcttccagagacgatagctgattgagtacaaagtccaatgattgcacgaagttcttcggcggtttt
catggagtcatttctgatgaaacatttaatgatctaaatttcagtttatgtttgtaccccgtattcatacttaacaaattggtatt
acataccattaataatgcaagcataaaaaatcgttagtagatgtttctaaatataggttccgtaagcaaagaatataag
aatgaagcggtaatgataaaatcaatcgttatctaaaatgatcatactcatttattttattctattatattaacacatacattttt
aacagcaacacattcaatattgtattgttatttttatattatttacacaattaacaatatattattagtttatattactgaattaata
atataaaattcccaatcttgtcataaacacacactgagaaacagcataaacacaaaatccatcaaaaatgttgataa
attatctgatgttgttgttcgctgctatgataatcagatcattcgccgatagtggtaacgctatcgaaacgacatcgccag
aaattacaaacgctacaacagatattccagctatcagattatgcggtccagagggagatggatattgtttacacggtga
ctgtatccacgctagagatatcgacggtatgtattgtagatgctctcatggttatacaggcattagatgtcagcatgtagta
ttagtagactatcaacgttcagaaaaaccaaacactacaacgtcatatatcccatctcccggtattatgcttgtattagta
ggcattattattattacgtgttgtctattatctgtttataggttcactcgacgaactaaactacctatacaagatatggttgtgc
cataatttttataaattttttttatgagtatttttacaaaaatgtataaagtgtatgtcttatgtatatttataaaaatgctaaatatg
cgatgtatctatgttatttgtatttatctaaacaatacctctacctctagatattatacaaaaattttttatttcggcatattaaag
taaaatctagttaccttgaaaatgaatacagtgggtggttccgtatcaccagtaagaacataatagtcgaatacagtat
ccgattgagattttgcatacaatactagtctagaaagaaatttgtaatcatcttctgtgacgggagtccatatatctgtatca
tcgtcccatgctatattcctgttatcatcattagttaatgaaaataactctcgtgcttcagaaaagtcaaatattgtatccata
catacatctccaaaactatcgcttatacgtttatctttaacgatacctatacctagatggttatttactaacagacattttcca
gatctattgactataactcctatagtttccacatcaaccaagtaatgatcatctattgttatataacaataacataactctttt
ccgtttttatcagtatgtatatctatatcaacgtcgtcgttgtagtgaatagtagtcattgatctattatatgaaacggatatgt
ctagaacggcaattgttttacgtccagttaacactttcgttgatttaaagtctagagtctttgcaaacataatatccttatccg
actttatatttcctgtagggtggtataattttattttgcctccacatatcggtgtttccaaatatattactagacaatattccatat
agttattagttaagggtacccaattagaacacgtacgcttattatcatcatttggatcgtatttcataaaagttattgtactat
cgatgtcaacacattctacattttttaatcgtctatatagtatttttctgatattttctataatatcagaattgtcttccatcggaag
ttgtatactatcggaatcagttacatgtttaaataattctctgatgtcattccttatacaatcaaattcattattaaacagtttaa
tagtctgtagacctttatcgtcgtaaatatccattgtcttattagttacgcttatttttatgtgtttttacgttgctttattatattttata
agaatgattgtttgacgaatcacgagaactattaagacacattattaggtatatattataaaaaagtttttgattacgatgtt
ataagaggaaagaggacacattaacatcatacatcaattaactacattcttataacatcgtaatcaaaagaattgcaa
ttttgatgtataacaactgtcaatgggttatggaattgtatattacatattatacggtatgttggtaacgacaaataccgatc
ggtaattgtctgccggtgtaatagaattatatatatctatctattacaccggccttgtatacataataataagttgtggtagta
tgatctccatatttataatttaggactttgtattcagtttttttggaatcataaaaaataaaaaaaagttttactaatttaaaatt
atttacatttttttcactgtttagtcgcggatatggaattcgatcctgccaaaatcaatacatcatctatagatcatgtaacaa
tattacaatacatagatgaaccaaatgatataagactaacagtatgcattatcacaaaaataaatccacatttggctaa
tcaatttcgggcttggaaaaaacgtatcgccggaagggactatatgactaacttatctagagatacaggaatacaaca
atcaaaacttactgaaactgtcaaaaaaatagaaacatatatggtctatatatacactacaatttagttattaattggata
accgatgtgattatcaatcaatattaagaaggttggtaaattggtacatagctaataatacctatacacccaataataca
acaaccatttctgagttggatatcatcaaaatactggataaatacgaggacgtgtatagagtaagtaaagaaaaaga
atgtgaaatttgctatgaagttgtttactcaaaacgatagatactttggtttattggattcgtgtaatcatatattttgcataaca
tgcatcaatatatggcatagaacacgaagagaaaccggtgcgtcggataattgtcctatatgtcgtacccgttttagaa
acataacaatgagcaagttaactaataaataaaaagtttaatttgttgacgacgtatgtcgttattttttctcgtataaaaga
ttaatttgattctaatataatctttagtattggataaatatcaattcaaattaattccattagattatatcataaataaaaatagt
agcacgcactacttcagccaaatattcttttttgaaacgccatctatcgtagtgaggacacaagtgaacctataatgagc
aaatttattagtatcggttacatgaaggactttacgtagagtggtgattccactatctgtggtacgaacggtttcatcttcttt
gatgccatcacccagatgttctataaacttggtatcctttgccaaccaatacatatagctaaactcaggcatatgttccac
acatcctgaacaatgaaattctccagaagatgttacaatgtctagatttggacatttggtttcaaccgcgttaacatatga
gtgaacacacccatacatgaaagcgatgagaaataggattttcatcttgccaaaatatcactagaaaaaatttatttatc
aattttaaaggtataaaaaatacttattgttgctcgaatattttgtatttgatggtatacggaagattagaaatgtaggtattat
catcaactgattctatggttttatgtattctatcatgtttcactattgcgttggaaataatatcatatgcttccacatatattttatttt
gttttaactcataatactcacgtaattctggattattgacatatctatgaataattttagctccatgatcagtaaatattaatga
gaacatagtattaccacctaccattatttttttcatctcattcaattcttaattgcaaagatctatataatcattatagcgttgac
ttatggactctggaatcttagacgatgtacagtcatctataatcatggcatatttaatacattgttttatagcatagtcgttatc
tacgatgttagatatttctctcaatgaatcaatcacacaatctaatgtaggtttatgacataatagcattttcagcagttcaat
gtttttagattcgttgatggcaatggctatacatgtatatccgttatttgatctaatgttgacatctgaaccggattctagcagt
aaagatactagagattgtttattatatctaacagccttgtgaagaagtgtttctcctcgtttgtcaatcatgttaatgtctttaa
gataaggtaggcaaatgtttatagtactaagaattgggcaagcataagacatgtcacaaagaccctttttgtatgtataa
gtgtaaaaattataacattcatagttggatttacataggtgtccaatcgggatctctccatcatcgagataattgatggcat
ctcccttccttttttagtagatatttcatcgtgtaagaatcaatattaatatttctaaagtatccgtgtatagcctctttatttacca
cagttccatattccactagagggatatcgccgaatgtcatatactcaattagtatatgttggaggacatccgagttcattgt
tttcaatatcaaaaagatggtttccttatcatttctccatagtggtacaatactacacattattccgtgcggctttccattttcca
aaaacaatttgaccaaatctacatctttattgtatctataatcactatttagataatcagccataattactcgagtgcaacat
gttagatcgtctatatatgaataagccgtgttatctattcctttcattaacaatttaacgatgtctatatctatatgagatgactt
aatataatattgaagagctgtacaatagtttttatctataaaagacggcttgattccgtgattaattagacatttaacaactt
ccggacgcacatatgctctcgtatccgactctgaatacagatgagagatgatatacagatgcaatacggtaccgcaat
ttcgtagttgataatcatcatacgcgtatcagtactcgtcctcataaagaacactgcagccattttctatgaacaaatcaat
aatttcaggaacaggatcatctgtcattacataattttctataactgaacgatggttttcacatttaacactcaagtcaaatc
catgttctaccaacacctttatcaagtcaacgtctacatttttggatttcatatagctgaatatattaaagtcatttatgttgcta
aatccagtggcttctagtagagccatcgctatatcctttaactttaacatgtctactatttgtgtattcttctaatggggtagct
gtctccaatttttgcgtaatggattagtgccactgtctagtagtagtttgacgacctcgacattattacaatgctcattaaaa
aggtatgcgtgtaaagcattattcttgaattggttcctggtatcattaggatctctgtctctcaacatctgtttaagttcatcga
gagccacctcctcattttccaaatagtcaaacattttgactgaatgagctactgtgaactctatacacccacacaactaa
tgtcattaaatatcatgtcaaaaacttgtacaattattaataaaaataatttagtgtttaaattttaccagttccagattttaca
cctccgttaaccccactttttacaccactggacgatcctcctccccacattccaccgccaccagatgtataagttttagat
cctttattactaccatcatgtccatggataaagacactccacatgccgccactaccccctttagaagacatattaataag
acttaaggacaagtttaacaataaaattaatcacgagtaccctactaccaacctacactattatatgattatagtttctattt
ttacagtaccttaactaaagtctctagtcacaagagcaatactaccaacctacactattatatgattatagtttctatttttat
aggaacgcgtacgagaaaatcaaatgtctaatttctaacggtagtgttgataaacgattatcgtcaatggatacctcctc
tatcatgtcgtctattttcttactttgttctattaacttattagcattatatattatttgattataaaacttatattgcttattagcccaa
tctgtaaatatcggattattaacatatcgtttctttgtaggtttatttaacatgtacatcactgtaagcatgtccgtaccatttattt
taatttgacgcatatccgcaatttctttttcgcagtcggttataaattctatatatgatggatacatgctacatgtgtacttataa
tcgactaatatgaagtacttgatacatattttcagtaacgatttattattaccacctatgaataagtacctgtgatcgtctagg
taatcaactgttttcttaatacattcgatggttggtaatttactcagaataatttccaatatcttaatatataattctgctatttctg
ggatatatttatctgccagtataacacaaatagtaatacatgtaaacccatattttgttattatattaatgtctgcgccattatc
tattaaccattctactaggctgacactatgcgacttaatacaatgataaagtatactacatccatgtttatatcatcaatata
cggcttacaaagttttagtatcgataacacatccaactcacgcatagagaaggtagggaataatggcataatatttatt
aggttatcatcattgtcattatctacaactaagtttccattttttaaaatatactcgacaactttaggatctctattgccaaatttt
tgaaaatatttatttatatgcttaaatctatataatgtagctccttcatcaatcatacatttaataacattgatgtatactgtatg
ataagatacatattctaacaatagatcttgtatagaatctgtatatcttttaagaattgtggatattattacgtaaactattaca
caattctaaaatataaaacgtatcacggtcgaataatagttgatcaactatataattatcgattttgtgatttttcttcctaaa
ctgtttacgtaaatagttagatagaatattcattagttcatgaccactatagttactatcgaataacgcgtcaaatatttccc
gtttaatatcgcatttgtcaagataataatagagtgtggtatgttcacgataagtataataacgcatctctttttcgtgtgaaa
ttaaatagtttattacgtccaaagatgtagcataaccatcttgtgacctagtaataatataataatagagaactgttttacc
cattctatcatcataatcagtggtgtagtcgtaatcgtaattgtctaattcatcatcccaattataatattcaccagcacgtct
aatctgttctattttgatcttgtatccatactgtatgttgctacatgtaggtattcctttatccaataatagtttaaacacatctac
attgggatttgatgttgtagcgtatttttctacaatattaataccatttttgatactatttatttctatacctttcgaaattagtaattt
caataagtctatatcgatgttatcagaacatagatattcgagtatatcaaaatcattgatatttttatagtcgactgacgac
aataacaaaatcacaacatcgtttttgatattattatttttcttggtaacgtatgcctttaatggagtttcaccatcatactcata
taatggatttgcaccactttctatcaatgattgtgcactgctggcatcgatgttaaatgttttacaactatcatagagtatctta
tcgttaaccatgattggttgttgatgctatcgcattttttggtttctttcatttcagttatgtatggatttagcacgtttgggaagca
tgagctcatatgatttcagtactgtagtgtcagtactattagtttcaataagatcaatctctagatctatagaatcaaaacac
gataggtcagaagataatgaatatctgtaggcttcttgttgtactgtaacttctcgttttgttagatgtttgcatcgtgctttaac
atcaatggtacaaattttatcctcgctttgtgtatcatattcgtccctactataaaattgtatattcagattatcatgagatgtgt
atacgctaacggtatcaataaacggagcacaccatttagtcataaccgtaatccaaaaatttttaaagtatatcttaacg
aaagaagttgtgtcattgtctacggtgtatggtactagatcctcataagtgtatatatctagagtaatgtttaatttatcaaat
ggttgataatatggatcctcatgacaatttccgaagatggaaatgagatatagacatgcaataaatctaattgcggaca
tggttactccttaaaaaaatacgaataatcaccttggctatttagtaagtgtcatttaacactatactcatattaatccatgg
actcataatctctatacgggattaacggatgttctatatacggggatgagtagttttcttctttaactttatactttttactaatca
tatttagactgatgtatgggtaatagtgtttaaagagttcgttctcatcatcagaataaatcaatatctctgtttttttgttataca
gatgtattacagcctcatatattacgtaatagaacgtgtcatctaccttattaactttcaccgcatagttgtttgcaaatacg
gttaatcctttgacctcgtcgatttccgaccaatctgggcgtataatgaatctaaactttaatttcttgtaatcattcgaaata
atttttagtttgcatccgtagttatcccctttatgtaactgtaaatttctcaacgcgatatctccattaataatgatgtcgaattc
gtgctgtatacccatactgaatggatgaacgaataccgacggcgttaatagtaatttactttttcatctttacatattgggta
ctagttttactatcataagtttataaattccacaagctactatggaataagccaaccatcttagtataacacacatgtctta
aagtttattaattaattacatgttgttttatatatcgctacgaatttaaacagagaaatcagtttaggaaaaaaaaatatcta
tctacatcatcacgtctctgtattctacgatagagtgctactttaagatgagacatatccgtgtcatcaaaaatatactccat
taaaatgattattccggcagcgaacttgatattggatatatcacaacctttgttaatatctacgacaatagacagcagtcc
catggttccataaacagtgagtttatctttctttgaagagatattttgtagagatcttataaaactgtcgaatgacatcgcatt
tatatctttagctaaatcgtatatgttaccatcgtaatatctaaccgcgtctatcttaaacgtttccatcgctttaaagacgtttc
cgatagatggtctcatttcatcagtcatactgagccaacaaatataatcgtgtataacatctttgatagaatcagactcta
aagaaaacgaatcggctttattatacgcattcatgataaacttaatgaaaaatgtttttcgttgtttaagttggatgaatagt
atgtcttaataattgttattatttcattaattaatatttagtaacgagtacactctataaaaacgagaatgacataactagttat
caaagtgtctaggacgcgtaattttcatatggtatagatcctgtaagcattgtctgtattctggagctattttctctatatctaat
ttctgaacgttcaccaatgtctctagccactttggcactaatagcgatcattcgcttagcgtcttctatattattaactggttga
ttcaatctatctagcaatggaccgtcggacagcgtcattctcatgttcttaatcaatgtacatacatcgccgtcatctacca
attcatccaacaacataagctttttaaaatcatcattataataggtttgatcgttgtcatttctccaaagaatatatctaataa
gtagagtcctcatgattagttaacaactattttttatgttaaatcaattagtacaccgctatgtttaatacttattcatattttagtt
tttaggattgagaatcaatacaaaaattaatgcatcattaattttagaaatacttagtttccacgtagtcaatgaaacatttg
aactcatcgtacaggacgttctcgtacaggacgtaactataaaccggtttatatttgttcaagatagatacaaatccgat
aactttttttacgaattctacgggatccactttaaaagtgtcataccgggttctttttatttttttaaacagattaatggtgtgatg
ttgattaggtcttttacgaatttgatatagaatagcgtttacatattctccataatggtcaatcgccatttgttcgtatgtcataa
attctttaattatatgacactgtgtattatttagttcatccttgttcatcattaggaatctatccaatatggcaattatactagaac
tataggtgcgttgtatacacatattgatgtgtctgtttatacaatccatgctactaccttcgggtaaaattgtagcatcatata
ccatttctagtactttaggttcattgttatccattgcagaggacgtcatgaacgcatcctaaaaaaatatattatttttatgttat
tttgttaaaaataatcatcgaatacgaatcatccagtccactgaatagcaaaatctttactattttggtatcttccaatgtgg
ctgcctgatgtaatggaaattcattctctagaagatttttcaatgctccagcgttcaacaacgtacatactagacgcacgtt
attatcagctattgcataatacaaggcactatgtccatggacatccgccttaaatgcatctttgctagagagaaagctttt
cagctgcttagacttccaagtattaattcgtgacagatccatgtctgaaacgagacgctaattagtgtataatttttgtcata
ttgcaccagaattaataatatctctaatagatctgattagtagatacatggctatcgcaaaacaacatatacacatttaat
aaaaataatatttattaagaaaattcagatttcacgtacccatcaatataaataaaataatgattccttacaccgtaccca
tattaaggagattctaccttacccataaacaatataaatccagtaatatcatgtctgatgatgaacacaaatggtgtatta
aattccagtttttcaggagatgatctcgccgtagctaccataatagtagatgcctctgctacagttccttgttcgtcgacatc
tatctttgcattctgaaacattttataaatatataatgggtccctagtcatatgtttaaacgacgcattatctggattaaacat
actaggagccatcatttcggctatcgacttaatatccctcttattttcgatagaaaatttagggagtttaagattgtacacttt
attccctaattgaaacgaccaatagtctaattttgcagccgtaatagaatctgtgaaatgggtcatattatcacctattgcc
aggtacatactaatattagcatccttatacggaaggcgtaccatgtcatattctttgtcatcgattgtgattgtatttccttgca
atttagtaactacgttcatcatgggaaccgttttcgtaccgtacttattagtaaaactagcattgcgtgttttagtgatatcaa
acggatattgccatatacctttaaaatatatagtattaatgattgcccatagagtattattgtcgagcatattagaatctact
acattagacataccggatctacgttctactatagaattaattttattaaccgcatctcgtctaaagtttaatctatataggcc
gaatctatgatattgttgataatacgacggtttaatacacacagtattatctacgaaactttgataagttagatcagtgtac
gtatatttagatgttttcagcttagctaatcctgatattaattctgtaaatgctggacccagatctctttttctcaaatccatagt
cttcaataattctattctagtattacctgatgcaggcaatagcgacataaacatagaaaacgaataaccaaacggtga
gaagacaatattatcatcttgaatatttttatacgctactataccggcattggtaaatccttgtagacgataggtagacgct
gaacacgttaacgatagtatcaataacgcaatcatgattttatggtattaataattaaccttatttttatgttcggtataaaaa
ttattgatgtctacacatccttttgtaattgacatctatatatccttttgtataatcaactctaatcactttaacttttacagttttccc
taccagtttatccctatattcaacatatctatccatatgcatcttaacactctctgccaagatagcttcagagtgaggatagt
caaaaagataaatatatagagcataatcattctcgtatactctgccctttattacatcgcccgcattgggcaacgaataa
caaaatgcaagcatcttgttaacgggctcgtaaattgggataaaaattatgtttttatatctattttattcaagagaatattca
ggaatttctttttccggttgtatctcatcgcagtatatatcatttgtacattgtttcatattttttaatagtttacaccttttagtagga
ctagtatcgtacaattcatagctgtattttgaattccaatcacgcataaaaatatcttctaattgttgacgaagacctaatcc
atcatccggtgtaatattaatagatgctccacatgtatccgtaaagtaatttcctgtccaatttgaggtacctatataggcc
gttttatcggttaccatatatttggcatggtttaccctagaatacggaatgggaggatcagcatctggtacaataaatagc
tttacttctatatttatgtttttagattttagcatagcgatagatcttaaaaagtttctcatgataaacgaagatcgttgccagc
aactaatcaatagcttaacggatacttgtctgtctatagcggatcttcttaattcatcttctatataaggccaaaacaaaatt
ttacccgccttcgaataaataatagggataaagttcataacagatacataaacgaatttactcgcatttctaatacatga
caataaagcggttaaatcattggttctttccatagtacatagttgttgcggtgcagaagcaataaatacagagtgtggaa
caccacttacgttaatactaagaggatgatctgtattataatacgacggataaaagtttttccaattatatggtagattgtta
actccaagataccagtatacctcaaaaatttgagtgagatccgctgccaagttcctattattgaagatcgcaataccca
attctttgacctgagttagtgatctccaatccatgttagcgcttcctaaataaatatgtgtattatcagatatccaaaattttgt
atgaagaactcctcctaggatatttgtaatatctatgtatcgtacttcaactccggccatttgtagtctttcaacatcctttaat
ggtttgttagatttattgacggctactctaactcgtactcctcttttgggtaattgtacaatctcgtttaatattatcgtgccgaa
attcgtacccacttcatccgataaactccaataaaaagatgatatatctagtgtttttgtggtattggatagaatttccctcc
acatgttaaatgtagacaaatatactttatcaaattgcatacctataggaatagtctctgtaatcactgcgattgtattatcc
ggattcattttatttgttaaaagaataatcctatatcacttcactctattaaaaatccaagtttctatttctttcatgactgatttttt
aacttcatccgtttccttatgaagatgatgtttggcaccttcataaatttttatttctctattacaatttgcatgttgcatgaaata
atatgcacctaaaacatcgctaatctcattgtttgttccctggagtatgagagtcggggtgttaatcttggaaattatttttcta
accttgttggtagccttcaagacctgactagcaaatccagccttaattttttcatgattgattaatgggtcgtattggtatttat
aaactttatccatatctctagatactgattctggacatagctttccgactggcgcatttggtgtgatggttcccataagtttgg
cagctagcagattcagtcttgaaacagcatctgcattaactagaggagacattagaatcattgctgtaaacaagtttgg
attatcgtaagaggctagtatagaaattgttgctcccatggaatgacccaataagtagatttaatagttaccacgtgctgt
accaaagtcatcaatcatcattttttcaccattacttcttccatgtccaatatgatcatgtgagaatactaaaattcctaacg
atgatatgttttcagctagttcgtcataacgtccagaatgtttaccagctccatgacttatgaatactaatgccttaggatat
gtaatcattgtccagattgaacatacagtttgcactcatgattcacgttatataactatcaatattaacagttcgtttgatgat
catattatttttatgttttattgataattgtaaaaacatacaattaaatcaatatagaggaaggagacggctactgtcttttgt
gagatagtcatggcgactaaattagattatgaggatgctgttttttactttgtggatgatgataaaatatgtagtcgcgactc
catcatcgatctaatagatgaatatattacgtggagaaatcatgttatagtgtttaacaaagatattaccagttgtggaag
actgtacaaggaattgatgaagttcgatgatgtcgctatacggtactatggtattgataaaattaatgagattgtcgaagc
tatgagcgaaggagaccactacatcaattttacaaaagtccatgatcaggaaagtttattcgctaccataggaatatgt
gctaaaatcactgaacattggggatacaaaaagatttcagaatctagattccaatcattgggaaacattacagatctg
atgaccgacgataatataaacatcttgatactttttctagaaaaaaaattgaattgatgatataggggtcttcataacgca
taattattacgttagcattctatatccgtgttaaaaaaaattatcctatcatgtatttgagagttttatatgtagcaaacatgat
agctgtgatgccaataagctttagatattcacgcgtgctagtgttagggatggtattatctggtggtgaaatgtccgttatat
aatctacaaaacaatcatcgcatatagtatgcgatagtagagtaaacatttttatagtttttactggattcatacatcgtcta
cccaattcggttatgaatgaaattgtcgccaatcttacacccaaccccttgttatccattagtatagtattaacttcgttattta
tgtcataaactgtaaatgattttgtagatgccatatcatacatgatattcatgtccctattataatcattactaactttatcaca
atatatgttgataatatctatatatgatctagtctttgtgggcaactgtctatacaagtcgtctaaacgttgtttactcatatagt
atcgaacagccatcattacatggtcccgttccgttgatagataatcgagtatgttagtggacttgtcaaatctatataccat
attttctggaagtggatatacatagtcgtgatcaacattattgctagcctcatcttctatatcctgtactataccatctacata
atctacgatattattacacataaacatcgacaacatactattgtttattatctaagtcctgttgatccaaacccttgatctcct
ctatttgtactatctagagattgtacttcttccagttctggataatatatacgttgatagattagctgagctattctatctccagt
atttacattaaacgtacattttccattattaataagaatgactcctatgtttcccctataatcttcgtctattacaccacctccta
tatcaatgccttttagtgacagaccagacctaggagctattctaccatagcaaatcttaggcatggacatactaatatct
gtcttaattaactgtctttctcctggagggatagtataatcgtaagcgctatacaaatcatatccggcagcacccggcgat
tgcctagtaggagatttagctctgttagtttccttaacaaatctaactggtgagttaatattcatgttgaacataaaactaat
attttatttcaaaattatttaccatcccatatattccatgaataagtgtgatgattgtacacttctatagtatctatatacgattca
cgataaaatcctcctatcaatagcagtttattatccactatgatcaattctggattatccctcggataaataggatcatctat
cagagtccatgtattgctggattcacaataaaattccgcatttctaccaaccaagaataaccttctaccgaacactaac
gcgcatgatttataatgaggataataagtggatggtccaaactgccactgatcatgattgggtagcaaatattctgtagtt
gtatcagtttcagaatgtcctcccattacgtatataacattgtttatggatgccactgctggattacatctaggtttcagaag
actcggcatattaacccaagcagcatccccgtggaaccaacgctcaacagatgtgggatttggtagacctcctactac
gtataatttattgttagcgggtatcccgctagcatacagtctggggctattcatcggaggaattggaatccaattgtttgat
atataatttacagctatagcattgttatgtatttcattgttcatccatccaccgatgagatatactacttctccaacatgagta
cttgtacacatatggaatatatctataatttgatccatgttcataggatactctatgaatggatacttgtatgatttgcgtggtt
gtttatcacaatgaaatattttggtacagtctagtatccattttacattatttatacctctgggagaaagataatttgacctgat
tacatttttgataaggagtagcagatttcctaatttatttcttcgctttatataccacttaatgacaaaatcctcatctggaaca
tttagttcatcgctttctagaataagtttcatagatagataatcaaaattgtctatgatgtcatcttccagttccaaaaagtgtt
tggcaataaagtttttagtatgacataagagattggatagtccgtattctatacccatcatgtaacactcgacacaatattc
ctttctaaaatctcgtaggataaagtttatacaagtgtagatgataaattctacagaggttaatatagaagcacgtaataa
attgacgacgttatgactatctatatatacctttccagtatatgagtaaataactatagaagttagactgtgaatgtcaagg
tctagacaaaccctcgtaactggatctttatttttcgtgtatttttgacgtaaatgtgtgcgaaagtaaggagataactttttc
aatatcgtagaattgactattatattgcctcctatggcatcaataattgttttgaatttcttagtcatagacaatgctaatatatt
cttacagtacacagtattgacaaatatcggcatttatgtttctttaaaagtcaacatctaaagaaaaatgattatcttcttga
gacataactcccattttttggtattcacccacacgtttttcgaaaaaattagttttaccttctaatgatatattttccatgaaatc
aaacggattggtaacattataaatttttttaaatcccaattcagaaatcaatctatccgcgacgaattctatatatgttttcat
catttcacaattcattcctataagtttaactggaagagccgcagtaagaaattcttgttcaatggataccgcatctgttata
atagatctaacggtttcttcactcggtggatgcaataaatgtttaaacatcaaacatgcgaagtcgcagtgtagaccctc
gtctctactaatcaattcgttggaaaacgtgagtccgggcattaggccacgctttttaagccaaaatatggaagcgaat
gatccggaaaagaagattccttctactgcagcaaaggcaataagtctctctccataaccggcgctgtcatgtatccact
tttgagcccaatcggccttcttttttacacaaggcatcgtttctatggcattaaagagatagtttttttcattactatctttaacat
aagtatcgatcaaaagactatacatttccgaatgaatgttttcaatggccatctgaaatccgtagaaacatctagcctcg
gtaatctgtacttctgtacaaaatcgttccgccaaattttcattcactattccgtcactggctgcaaaaaacgccaatacat
gttttataaaatatttttcgtctggtgttagtttattccaatcattgatatctttagatatatctacttcttccactgtccaaaatgat
gcctctgcctttttatacatgttccagatgtcatgatattggattgggaaaataacaaatctatttggatttggtgcaaggat
gggttccataactaaattaacaataacaataaattttttttcagttatctatatgcctgtacttggatcttttgtacatcgatatc
gccgcaatcactacaataattacaagtattattgatagcattgttattagtactatcataattaaattatcgttattatcattttg
taattgtgacatcatactagataaatcgtttgcgagattgttgtgggaagcgggcatggaggatgaattatcgttattatta
tttaaagcctcccattcggattcacaaatatggcgcgcgttcaacattttatggaaacagataacaagaaaactcgtca
tcgttcaaatttttaacgatagtaaaccgattaaacgtcgagctaatttctaacgctagcgactctgttggatatgggtttcc
agatatatatcttttcagttcccctacgtatctataatcatctgtaggaaatggaagatatttccatttatctactgttcctaata
tcatatgtggtggtgtagtagaaccattaagcgcgaaagatgttatttcgcatcgtattttaacttcgcaataatttctggtta
gataacgcactctaccagtcaagtcaatgatattagcctttacagatatattcatagtagtcgtaacgatgactccatcttt
tagatgcgatactcctttgtatgtaccagaatcttcgtacctcaaactcgatatatttaaacaagttaatgagatattaacg
cgttttatgaatgatgatatataaccagaagttttatcctcggtggctagcgctataaccttatcattataataccaactagt
gtgattaatatgtgacacgttagtgtgggtacaaatatgtacattatcgtctacgtcgtattcgatacatccgcatacagcc
aacaaatataaaatgacaaatactctaacgccgttcgtacccatcttgatgcggtttaataaatgttttgatttcaatttattg
taaaaaaagattcggttttatactgttcgatattctcattgcttatattttcatctatcatctccacacagtcaaatccgtggtta
gcatgcacctcatcaaccggtaaaagactatcggactcttctatcattataactctagaatatttaatttggtcattattaat
caagtcaattatcttatttttaacaaacgtgagtattttactcattttttataaaaacttttagaaatatacagactctatcgtgt
gtctatatcttctttttatatccaatgtatttatgtctgatttttcttcatttatcatatataatggtccaaattctacacgtgcttcgg
attcatccagatcattaaggttcttataattgtaacatccttctcttccctcttctacatcttccttcttattcttattcttagcgtcac
agaatctaccacagcaggatcccatgacgagcgtcatattaaactaatccattttcaattataatatacgattagtaatg
accattaaaataaaaatattcttcataaccggcaagaaagtgaaaagttcacattgaaactatgtcagtagtatacatc
atgaaatgatgatatatatatactctattttggtggaggattatatgatataattcgtggataatcattcttaagacacatttct
tcattcgtaaatcttttcacgttaaatgagtgtccatattttgcaatttcttcatatgatggcggtgtacgtggacgaggctgct
cctgttcttgttgtagtcgccgactgtcgtgtctgcgtttagatccctccattatcgcgattgcgtagatggagtactattttata
ccttgtaattaaatttttttattaattaaacgtataaaaacgttccgtatctgtatttaagagccagatttcgtctaatagaaca
aatagctacagtaaaaataactagaataattgctacacccactagaaaccacggatcgtaatacggcaatcggttttc
gataataggtggaacgtatattttatttaaggacttaacaattgtctgtaaaccacaatttgcttccgcggatcctgtattaa
ctatctgtaaaagcatatgttgaccgggcggagccgaacattctccgatatccaatttctgtatatctataatattattaac
ctccgcatacgcattacagttcttttctagcttggataccgcactaggtacatcgtctagatctattcctatttcctcagcgat
agctcttctatccttttccggaagcaatgaaatcacttcaataaatgattcaaccatgagtgtgaaactaagtcgagaatt
actcatgcatttgttagttattcggagcgcgcaatttttaaactgtcctataacctctcctatatgaatagcacaagtgacat
tagtagggatagaatgttgagctaatttttgtaaataactatctataaaaagattatacaaagttttaaactctttagtttccg
ccatttatccagtctgagaaaatgtctctcataataaatttttccaagaaactaattgggtgaagaatggaaacctttaatc
tatatttatcacagtctgttttggtacacatgatgaattcttccaatgccgtactaaattcgatatctttttcgatttctggatatgt
ttttaataaagtatgaacaaagaaatggaaatcgtaataccagttatgtttaactttgaaattgttttttattttcttgttaatgat
tccagccacttgggaaaagtcaaagtcgtttaatgccgatttaatacgttcattaaaaacaaactttttatcctttagatga
attattattggttcattggaatcaaaaagtaagatattatcgggtttaagatctgcgtgtaaaaagttgtcgcagcatggta
gttcgtaaattttaatgtataacagagccatctgtaaaaagataaactttatgtattgtaccaaagatttaaatcctaatttg
atagctagctcggtatctactttatctgccgaatacagtgctaggggaaaaattataatatttcctctttcgtattcgtagtta
gttctcttttcatgttcgaaaaagtgaaacatgcggttaaaatagtttataacattaatattactgttaataactgccggata
aaagtgggatagtaatttcacgaatttgatactgtcctttctctcgttaaacgcctttaaaaaaactttagaagaatatctca
atgatagttcctgaccatccatagtttgtatcaataatagcaacatatgaagaacacgtttatacagagtatgtaaaaat
gttaatttatagtttaatcccatggcccacgcacacacgattaattttttttcatctccctttagattgttgtatagaaatttgggt
actgtgaactccgccgtagtttccatgggactatataattttgtggcctcgaatacaaattttactacatagttatctatctta
aagactataccatatcctcctgtagatatgtgataaaaatcgtcgtttataggataaaatcgtttatccttttgttggaaaaa
ggatgaattaatgtaatcattctcttctatctttagtagtgtttccttattaaaattcttaaaataatttaacaatctaactgatgg
agcccaattttggtgtaaatctaattgggacattatattgttaaaatacaaacagtctcctaatataacagtatctgataat
ctatggggagacatccattgatattcaggggatgaatcattggcaacacccatttattgtacaaaaagccccaatttac
aaacgaaagtccaggtttgatagagacaaactattaactattttgtctctgtttttaatttctttggtaatgaaattattcacaa
tatcagtatcttctttatctaccagagattttactaacttgataaccttggctgtctcattcaatagggtagtaatatttgtatgtg
tgatattgatatcttttagaagtgattctttgatggtgccagcatacgaattacaataatgcagaaactcggttaacatgca
ggaattatagtaagccaattccaattgttgcctgtgttgtattagagtgtcaatatgagcaatggtgtccttgcgtttctctga
tagaatgcgagcagcgattttggcgttatcatttgacgatatttctggaatgacgaatcctgtttctactaactttttggtagg
acaaagtgaaacaatcaagaagatagcttctcctcctatttgtggaagaaattgaactcctctagatgatctccttgaca
gatattggaccgaattacagaagtacctggaatgtaaagccctgaaaccccctcattttttaagcagattgttgccgtaa
atcctgcactatgcccaagatagagagctcctttggtgaatccatctctatgtttcagtttaaccaagaaacagtcagctg
gtctaaaatttccatctctatctaatacagcatctaacttgatgtcaggaactatgaccggttatgttatatgtaacattgagt
aaatccttaagttcataatcatcactgtcatcagttatgtacgatccaaacaatgtttctactggcatagtggatacgaag
atgctatccatcagaatgtttccctgattagtattttctatatagctattcttctttaaacgattttccaaatcagtaactatgttc
atttttttaggagtaggacgcctagccagtatggaagaggattttctagatcctctcttcaacatctttgatctcaatggaat
gcaaaaccccatagtgtaacaaccaacgataaaaataatattgtttttcactttttataattttaccatctgactcatggatt
cattaatatctttataagagctactaacgtataattctttataactgaactgagatatatacaccggatctatggtttccataa
ttgagtaaatgaatgctcggcaataactaatggcaaatgtataaaacaacgaaattatactagagttgttaaagttaat
attttctatgagctgttccaataaattatttgttgtaactgcgttcaagtcataaatcatcttgatactatccagtaaaccgttttt
aagttctggaatattattatcccattgtaaagcccctaattcgactatcgaatatcctgctctgatagcagtttcaatatcga
cggacgtcaatactgtaataaaggtggtagtattgtcatcatcgtgataaactactggaatatggtcgttagtaggtacg
gtaactttacacaacgcgatatataactttccttttgtaccatttttaacgtagttgggacgtcctgcagggtattgttttgaag
aaatgatatcgagaacagatttgatacgatatttgttggattcctgattatttactataatataatctagacagatagatgat
tcgataaatagagaaggtatatcgttggtaggataatacatccccattccagtattctcggatactctattaatgacacta
gttaagaacatgtcttctattctagaaaacgaaaacatcctacatggactcattaaaacttctaacgctcctgattgtgtct
cgaatgcctcgtacaaggatttcaaggatgccatagattctttgaccaacgatttagaattgcgtttagcatctgattttttta
ttaaatcgaatggtcggctctctggtttgctaccccaatgataacaatagtcttgtaaagataaaccgcaagaaaatttat
acgcatccatccaaataaccctagcaccatcggatgatattaatgtattattatagattttccatccacagttattgggcca
gtatactgttagcaacggtatatcgaatagattactcatgtaacctactagaatgatagttcgtgtactagtcataatatctt
taatccaatctaagaaatttaaaattagattttttacactgttaaagttaacaaaggtattacccggatacgtggatatcat
atatggtattggtccattatcagtaatagctccataaactgatacggcgatggtttttatatgtgtttgatctaacgaggaag
aaattcgcgcccacaattcatctctagatatgtatttaatatcaaacggtaacacatcaatttcgggacgcgtatatgtttc
taaatttttaatccaaatataatgatgacctatatgccctattatcatactgtcaactatagtacacctagagaacttacgat
acatctgtttcctataatcgttaaattttacaaatctataacatgctaaaccttttgacgacaaccattcattaatttctgatat
ggaatctgtattctcaataccgtatcgttctaaagccagtgctatatctccctgttcgtgagaacgctttcgtataatatcgat
caacggataatctgaagtttttggagaataatatgactcatgatctatttcgtccataaacaatctagacataggaattgg
aggcgatgatcttaattttgtgcaatgagtcgtcaatcctataacttctaatattgtaatattcatcatcgacataacactatc
tatgttatcatcgtatattagtataccatgaccttcttcatttcgtgccaaaatgatatacagtcttaaatagttacgcaatatc
tcaatagtttcataattgttagctgttttcatcaaggtttgtatcctgtttaacatgatggcgttctatacgtttctattttttaaatttt
taacgatttactgtggctagatacccaatctctctcaaatatttttttagcctcgcttacaagctgtttatctatactattaaaac
tgacgaatccgtgattttggtaatgggttccgtcgaaatttgccgaagtgatatgaacatattcgtcgtcgactatcaaca
attttgtattattctgaatagtgaaaaccttcacagatagatcattttgaacacacaacgcgtctagacttctggcggttgc
catagaatatacgtcgttcttatcccaattaccaactagaagtctgatcttaactcctctattaatggctgcttctataatgga
gttgtaaatgtcgggccaatagtagctattaccgtcgacacgtgtagtgggaactatggccaaatgttcaatatctatact
agtcttagccgacttgagtttatcaataactacatcagtgtctagatctctagaatatcccaataggtgttccggagaatc
agtaaagaacactccacctataggattcttaatatgatacgcagtgctaactggcagacaacaagccgcagagcat
aaattcaaccatgaattttttgcgctattaaaggctttaaaagtatcaaatcttctacgaagatctgtggccagcggggga
taatcagaatatacacctaacgttttaatcgtatgtatagatcctccagtaaatgacgcgtttcctacataacatctttcatt
atctgacacccaaaaacaaccgagtagtagtcccacattattttttttatctatattaacggttataaaatttatatccgggc
agtgactttgtagctctcccagatttcttttccctcgttcatctagcaaaactattattttaatccctttttcagatgcctcttttagt
ttatcaaaaataagcgctcccctagtcgtactcagaggattacaacaaaaagatgctatgtatatatatttcttagctaga
gtgataatttcgttaaaacattcaaatgttgttaaatgatcggatctaaaatccatattttctggtagtgtttctaccagcctac
attttgctcccgcaggtaccggtgcaaatggccacatttagttaacataaaaacttatacatcctgttctatcaacgattct
agaatatcatcggctatatcgctaaaattttcatcaaagtcgacatcacaacctaactcagtcaatatattaagaagttc
catgatgtcatcttcgtctatttctatatccgtatccattgtagattgttgaccgattatcgagtttaaatcattactaatactca
atccttcagaatacaatctgtgtttcattgtaaatttataggcggtgtatttaagttggtagattttcaattatgtatcaatatag
caacagtagttcttgctcctccttgattctagcatcctcttcattattttcttctacgtacataaacatgtccaatacgttagac
aacacaccgacgatggcggccgccacagacacgaatatgactaaaccgatgaccatttaaaaacccctctctagct
ttcacttaaactgtatcgattattcttttagaacatgtataatataaaaacattattctatttcgaatttaggcttccaaaaatttt
tcatccgtaaaccgataataatatatatagacttgttaatagtcggaataaatagattaatgcttaaactatcatcatctcc
acgattagagatacaatatttacattttttttgctgtttcgaaactttatcaatacacgttaatacaaacccaggaaggaga
tattgaaactgaggctgttgaaaatgaaacggtgaatacaataattcagataatgtaaaatcatgattccgtattctgatg
atattagaactgctaatggatgtcgatggtatgtatctaggagtatctattttaacaaagcatcgatttgctaatatacaatt
atcattttgattaattgttattttattcatattcttaaaaggtttcatatttatcaattcttctacattaaaaatttccatttttaatttatg
tagccccgcaatactcctcattacgtttcattttttgtctataatatccattttgttcatctcggtacatagattatccaattgaga
agcgcatttagtagttttgtacattttaagtttattgacgaatcgtcgaaaactagttatagttaacattttattatttgataccct
gatattaatacccctgccgttactattatttataactgatgtaatccacgtaacattggaattaactatcgatagtaatgcat
cgacgcttccaaaattgtctattataaactcaccgataatttttttattacatgttttcatattcattaggattattaaatctttaat
cttactacgattgtatgcgttgatattgcaagacgtcattctaaaagacggaggatctccatcaaatgccagacaatca
cgtacaaagtacatggaaataggttttgttctattgcgcatcatagatttatatagaacacccgtagaaatactaatttgttt
tactctataaaatactaatgcatctatttcatcgttttgtataacgtctttccaagtgtcaaattccaaatttttttcattgatagta
ccaaattcttctatctctttaactacttgcatagataggtaattacagtgatgcctacatgccgttttttgaaactgaatagat
gcgtctagaagcgatgctacgctagtcacaatcaccactttcatatttagaatatatatatgtaaaaatatagtagaatttc
attttgttttttttctatgctataaatgaattctcattttgcatctgctcatactccgttttatatcaataccaaagaaggaagatat
ctggttctaaaagccgttaaagtatgcgatgttagaactgtagaatgcgaaggaagtaaagcttcctgcgtactcaaa
gtagataaaccctcatcacccgcgtgtgagagaagaccttcgtccccttccagatgcgagagaatgaataacccag
gaaaacaagttccgtttatgaggacggacatgctacaaaatatgttcgcggctaatcgcgataatgtagcttctagactt
ttgtcctaaaatacaattatatccttttcgatattaataaatccgtgtcgtccaggttttttatctctttcagtatgtgaatagata
ggtattttatctctattcatcatcgaatttaagagatccgataaacattgtttgtattctccagatgtcagcatctgatacaac
aatatatgtgcacataaacctctggcacttatttcatgtaccttccccttatcactaaggagaatagtatttgagaaatatgt
atacatgatattatcatgaattagatatacagaatttgtaacactctcgaaatcacacgatgtgtcggcgttaagatctaa
tatatcactcgataacacattttcatctagatacactagacattttttaaagctaaaatagtctttagtagtaacagtaactat
gcgattattttcatcgatgatacatttcatcggcatattattacgcttaccatcaaagactataccatgtgtatatctaacgta
ttctagcatggttgccatacgcgcattaaacttttcaggatctttggatagatcttccaatctatctatttgagaaaacattttt
atcatgttcaatagttgaaacgtcggatccactatatagatattatctataaagattttaggaactacgttcatggtatcctg
gcgaatattaaaactatcaatgatatgattatcgttttcatcttttatcaccatatagtttctaagatatgggattttacttaatat
aatattatttcccgtaataaattttattagaaatgccaaatctataagaaaagtcctcgaattagtttgaagaatatctatat
cgccgtaccgtatatttggattaattagatatagagaatatgatccgtaacatatacaacttttattatggcgtctaagatat
tcttccatcaacttattaacatttttgactagggaagatacattatgacgtcccattacttttgccttgtctattactgcgacgtt
catagaatttagcatatctcttgccaattcttccattgatgttacattataagaaattttagatgaaattacatttggagcttta
atagtaagaactcctaatatgtccgtgtatgtggtcactaatacagattgtagttctataatcgtaaataatttacctatatta
tatgtttgagtctgtttagaaaagtagctaagtatacgatcttttatttctgatgcagatgtatcaacatcggaaaaaaatctt
tttttattcttttttactaaagatacaaatatgtctttgttaaaaacagttattttctgaatatttctagcttgtaattttaacatatgat
attcgttcacactaggtactctgcctaaataggtttctataatctttaatgtaatattaggaaaagtattctgatcaggattcct
attcattttgaggatttaaaactctgattattgtctaatatggtctcaacacaaactttttcacagagcgatagagtttttgata
actcgtttttcttaagaaatataaaactactgtctccagagctcgctctatcttttattttatttaattcgatacaaactcctgat
actggttcagaaagtaattcattaattttcagtcctttatagaagatatttaatatagataatacaaaatcttcagtttttgatat
cgatctgattgatcctagaactagatatattaataacgtgctcattaggcagtttatggcagcttgataattagatatagtat
attccagttcatatttattagataccgcattgcccagattttgatattctatgaattcctctgaaaataaatccaaaataacta
gacattctattttttgtggattagtgtactctcttccctctatcatgttcactactggtgtccacgatgataaatatctagaggg
aatataatatagtccataggatgccaatctagcaatgtcgaataactgtaattttattcttcgctcttcattatgaattgattctt
gaggtataaacctaacacaaattatattattagacttttcgtatgtaatgtctttcatgttataagtttttaatcctggaataga
atctattttaatgaggcttttaaacgcagagttctccaacgagtcaaagcataatactctgttggttttcttatatacgatgtta
cgattttcttctttgaatggaataggtttttgaattagtttataattacaacataatagataaggaagtgtgcaaatagtacg
cggaaaaaacataatagctcccctgttttcatccatggttttaagtaaatgatcactggcttctttagtcaatggatattcga
acattaaccgtttcatcatcattggacagaatccatatttcttaatgtaaagagtgatcaaatcattgtgtttattgtaccatct
tgttgtaaatgtgtattcggttatcggatctgctcctttttctattaaagtatcgatgtcgatctcgtctaagaattcaactatatc
gacatatttcatttgtatacacataaccattactaacgtagaatgtataggaagagatgtaacgggaacagggtttgttg
attcgcaaactattctaatacataattcttctgttaatacgtcttgcacgtaatctattatagatgccaagatatctatataatt
attttgtaagatgatgttaactatgtgatctatataagtagtgtaataattcatgtatttcgatatatgttccaactctgtctttgtg
atgtctagtttcgtaatatctatagcatcctcaaaaaatatattcgcatatattcccaagtcttcagttctatcttctaaaaaat
cttcaacgtatggaatataataatctattttacctcttctgatatcattaatgatatagtttttgacactatcttctgtcaattgatt
cttattcactatatctaagaaacggatagcgtccctaggacgaactactgccattaatatctctattatagcttctggacat
aattcatctattataccagaattaatgggaactattccgtatctatctaacatagttttaagaaagtcagaatctaagacct
gatgttcatatattggttcatacatgaaatgatctctattgatgatagtgactatttcattctctgaaaattggtaactcattcta
tatatgctttccttgttgatgaaggatagaatatactcaatagaatttgtaccaacaaactgttctcttatgaatcgtatatca
tcatctgaaataatcatgtaaggcatacatttaacaattagagacttgtctcctgttatcaatatactattcttgtgataattta
tgtgtgaggcaaatttgtccacgttctttaattttgttatagtagatatcaaatccaatggagctacagttcttggcttaaaca
gatatagtttttctggaacgaattctacaacattattataaaggactttgggtagataagtgggatgaaatcctattttaatt
aatgcgatagccttgtcctcgtgcagatatccaaacgcttttgtgatagtatggcattcattgtctagaaacgctctacga
atatctgtgacagatatcatctttagagaatatactagtcgcgttaatagtactacaatttgtattttttaatctatctcaataa
aaaaattaatatgtatgattcaatgtataactaaactactaactgttattgataactagaatcagaatctaatgatgacgta
accaagaagtttatctactgccaatttagctgcattatttttagcatctcgtttagattttccatctgccttatcgaatactcttcc
gtcgatatctacacaggcataaaatgtaggagagttactaggccccactgattcaatacgaaaagaccaatctctctt
agttatttggcagtactcattaataatggtgacagggttagcatctttccaatcaataatttttttagccggaataacatcatc
aaaagacttatgatcctctctcattgatttttcgcgggatacatcatctattatggcgtcagccataacatcagcatccggc
ttatccgcctccgttgtcataaaccaacgaggaggaatatcgtcggagctgtacaccatagcactacgttgaagatcgt
acagagctttattaacttctcgcttctccatattaagttgtctagttagttgtgcagcagtagctccttcgattccaatgttttta
atagccgcacacacaatctctgcgtcagaacgctcgtcaatatagatcttagacatttttagagagaactaacacaac
cagcaataaaactaatttattttatcatttttttattcatcatcctctggtggttcgtcgtttctatcgaatgtggatctgattaacc
cgtcatctataggtgatgctggttctggagattctggaggagatggattattatctggaagaatctctgttatttccttgttttc
atgtatcgattgcgttgtaacattaagattgcgaaatgctctaaatttgggaggcttaaagtgttgtttgcaatctctacacg
catgtctaactagtggaggttcgtcagcggctctagtttgaatcatcatcggcgtagtattcctacttttacagttaggaca
cggtgtattgtatttctcgtcgagaacgttaaaataatcgttgtaactcacatcctttattttatctatattgtattctactcctttct
taatgcattttataccgaataagagatagcgaaggaattctttttcggtgccgctagtacccttaatcatatcacatagtgtt
ttatattccaaatttgtggcaatagacggtttatttctatacgatagtttgtttctggaatcctttgagtattctataccaatattat
tctttgattcgaatttagtttcttcgatattagattttgtattacctatattcttgatgtagtactttgatgatttttccatggcccattct
attaagtcttccaagttggcatcatccacatattgtgatagtaattctcggatatcagtagcggctaccgccattgatgtttg
ttcattggatgagtaactactaatgtatacattttccatttataacacttatgtattaactttgttcatttatattttttcattattatgtt
gatattaacaaaagtgaatatatatgttaataattgtattgtggttatacggctacaatttcataatgagtggaagtcagtgt
ccgatgatcaatgacgatagctttactctgaaaagaaagtatcaaatcgatagtgcggagtcaacaataaaaatgga
taagaagaggataaagtttcagaatagagccaaaatggtaaaagaaataaatcagacaataagagcagcacaa
actcattacgagacattgaaactaggatacataaaatttaagagaatgattaggactactactctagaagatatagca
ccatctattccaaataatcagaaaacttataaactattctcggacatttcagccatcggcaaagcatcacagaatccga
gtaagatggtatatgctctgctgctttacatgtttcccaatttgtttggagatgatcatagattcattcgttatagaatgcatcc
aatgagtaaaatcaaacacaagatcttctctcctttcaaacttaatcttattagaatattagtggaagaaagattctataat
aatgaatgcagatctaataaatggagaataattggaacacaagttgataaaatgttgatagctgaatctgataaatata
caatagatgcaaggtataacctaaaacccatgtatagaatcaagggaaaatctgaagaagataccctctttatcaaa
cagatggtagaacaatgtgtgacatcccaggaattggtggaaaaagtgttgaagatactgtttagagatttgttcaaga
gtggagaatacaaagcgtacagatacgatgatgatgtagaaaatggatttattggattggatacactaaaattaaaca
ttgttcatgatatagttgaaccatgtatgcctgttcgtaggccagtggctaagatactgtgtaaagaaatggtaaataaat
actttgagaatccgctacatattattggtaaaaatcttcaagagtgcattgactttgttagtgaataggcatttcatctttctcc
aatactaattcaaattgttaaattaataatggatagtataaatagttattagtgataaaatagtaaaaataattattagaat
aagagtgtagtatcatagataactctcttctataaaaatggattttattcgtagaaagtatcttatatacacagtagaaaat
aatatagattttttaaaggatgatacattaagtaaagtaaacaattttaccctcaatcatgtactagctctcaagtatctagt
tagcaattttcctcaacacgttattactaaggatgtattagctaataccaatttttttgttttcatacatatggtacgatgttgta
aagtgtacgaagcggttttacgacacgcatttgatgcacccacgttgtacgttaaagcattgactaagaattatttatcgtt
tagtaacgcaatacaatcgtacaaggaaaccgtgcataaactaacacaagatgaaaaatttttagaggttgccgaat
acatggacgaattaggagaacttataggcgtaaattatgacttagttcttaatccattatttcacggaggggaacccatc
aaagatatggaaatcatttttttaaaactgtttaagaaaacagacttcaaagttgttaaaaaattaagtgttataagattac
ttatttgggcttacctaagcaagaaagatacaggcatagagtttgcggataatgatagacaagatatatacactctattt
caacaaactggtagaatagtccatagcaatctaacagaaacgtttagagattatatctttcccggagataagactagct
attgggtgtggttaaacgaaagtatagctaatgatgcggatattgttcttaatagacacgccattaccatgtatgataaaa
ttcttagttatatatactctgagataaaacaaggacgcgttaataaaaacatgcttaagttagtttatatctttgagcctgaa
aaagatatcagagaacttctgctagaaatcatatatgatattcctggagatatcctatctattattgatgcaaaaaacga
cgattggaaaaaatattttattagtttttataaagctaattttattaacggtaatacatttattagtgatagaacgtttaacgag
gacttattcagagttgttgttcaaatagatcccgaatatttcgataatgaacgaattatgtctttattctctacgagtgctgcg
gacattaaacgatttgatgagttagatattaataacagttatatatctaatataatttatgaggtgaacgatatcacattag
atacaatggatgatatgaagaagtgtcaaatctttaacgaggatacgtcgtattatgttaaggaatacaatacatacctg
tttttgcacgagtcggatcccatggtcatagagaacggaatactaaagaaactgtcatctataaaatccaagagtaga
cggctgaacttgtttagcaaaaacattttaaaatattatttagacggacaattggctcgtctaggtcttgtgttagatgattat
aaaggagacttgttagttaaaatgataaaccatcttaagtctgtggaggatgtatccgcattcgttcgattttctacagata
aaaaccctagtattcttccatcgctaatcaaaactattttagctagttataatatttccatcatcgtcttatttcaaaggtttttg
agagataatctatatcatgtagaagaattcttggataaaagcatccatctaaccaagacggataagaaatatatacttc
aattgataagacacggtagatcatagaacagaccaaatatattattaataatttgtatatacatagatataattatcacac
atttttgataaatgggaactgctgcaacaattcagactcccaccaaattaatgaataaagaaaatgcagaaatgatttt
ggaaaaaattgttgatcatatagttatgtatattagtgacgaatcaagtgattcagaaaataatcctgaatatattgattttc
gtaacagatacgaagactatagatctctcattataaaaagtgatcacgagtttgtaaagctatgtaaaaatcatgcaga
gaaaagttctccagaaacgcaacaaatgattatcaaacacatatacgaacaatatcttattccagtatctgaagtacta
ttaaaacctataatgtccatgggtgacataattacatataacggatgtaaagacaatgaatggatgctagaacaactct
ctaccctaaactttaacaatctccgcacatggaactcatgtagcataggcaatgtaacgcgtctgttttatacattttttagtt
atctgatgaaagataaactaaatatataagtataatcccattctaatactttaacctgatgtattacctgcatcttattagaa
tattaacctaactaaaagacataacatagttgataaaaagcggtaggatataaatattatggctgccaccgttccgcgtt
ttgacgacgtgtacaaaaatgcacaaagaagaattctagatcaagaaacattttttagtagaggtctaagtagaccgtt
aatgaaaaacacatatctatttgataattacgcgtatggatggataccagaaactgcaatttggagtagtagatacgca
aacttagatgcaagtgactattatcccatttcgttgggattacttaaaaagttcgagtttctcatgtctctatataaaggtcct
attcccgtatatgaagaaaaagtaaatactgaattcattgctaatggatcgttctctggtagatacgtatcatatcttcgaa
agttttctgcccttccaacaaacgagtttattagttttttgttactgacttccattccaatctataatatcttgttctggtttaaaaa
tactcagtttgatattactaaacacacattattcagatacgtctatacagataatgccaaacacctggcgttggctaggta
tatgcatcaaacaggagactataagcctttgtttagtcgtctcaaagagaattatatatttaccggtcccgttccaataagt
atcaaagatatagatcaccctaatcttagtagagcaagaagtccatccgattatgagacattagctaatattagtactat
attgtactttaccaagtatgatccggtattaatgtttttattgttttacgtacctgggtattcaattactacaaaaattactccag
ccgtagaatatctaatggataaactgaatctaacaaagagcgacgtacaactgttgtaaattattttatgcttcgtaaaat
gtaggttttgaaccaaacattctttcaaagaatgagatgcataaaactttattatccaatagattgactatttcggacgtca
atcgtttaaagtaaacttcgtaaaatattctttgatcactgccgagtttaaaacttctatcgataattgtttcatatgttttaatat
ttacaagttttttggtccatggtacattagccggacaaatatatgcaaaataatatcgttctccaagttctatagtttctggatt
atttttattatattcagtaaccaaatacatattagggttatctgcggatttataatttgagtgatgcattcgactcaacataaat
aattctagaggagacgatctactatcaaattcggatcgtaaatctgtttctaaagaacggagaatatctatacatacctg
attagaattcatccgtccttcagacaacatctcagacagtctggtcttgtatgtcttaatcatattcttatgaaacttggaaa
catctcttctagtttcactagtacctttattaattctctcaggtacagattttgaattcgacgatgctgagtatttcatcgttgtat
atttcttcttcgattgcataatcagattcttatataccgcctcaaactctattttaaaattattaaacaatactctattattaatca
gtcgttctaactctttcgctatttctatagacttatcgacatcttgactgtctatctctgtaaacacggagtcggtatctccata
cacgctacgaaaacgaaatctgtaatctataggcaacgatgttttcacaatcggattaatatctctatcgtccatataaa
atggattacttaatggattggcaaaccgtaacataccgttagataactctgctccatttagtaccgattctagatacaaga
tcattctacgtcctatggatgtgcaactcttagccgaagcgtatgagtatagagcactatttctaaatcccatcagaccat
atactgagttggctactatcttgtacgtatattgcatggaatcatagatggccttttcagttgaactggtagcctgttttagcat
ctttttatatctggctctctctgccaaaaatgttcttaatagtctaggaatggttccttctatcgatctatcgaaaattgctatttc
agagatgaggttcggtagtctaggttcacaatgaaccgtaatatatctaggaggtggatatttctgaagcaagagctga
ttatttatttcttcttccaatctattggtactaacaacgacaccgactaatgtttccggagatagatttccaaagatacacac
attaggatacagactgttataatcaaagattaatacattattactaaacattttttgttttggagcaaataccttaccgccttc
ataaggaaacttttgttttgtttctgatctaactaagatagttttagtttccaacaatagctttaacagtggacccttgatgact
gtactcgctctatattcgaataccatggattgaggaagcacatatgttgacgcacccgcgtctgtttttgtttctactccata
atactcccacaaatactgacacaaacaagcatcatgaatacagtatctagccatatctaaagctatgtttagattataat
ccttatacatctgagctaaatcaacgtcatcctttccgaaagataatttatatgtatcattaggtaaagtaggacatgatag
tacgactttaaatccattttcccaaatatctttacgaattactttacatataatatcctcatcaacagtcacataattacctgtg
gttaaaacctttgcaaatgcagcggctttgcctttcgcgtccgtagtatcgtcaccgatgaacgtcatttctctaactcctct
atttaatactttacccatgcaactgaacgcgttcttggatatagaatccaatttgtacgaatccaatttttcagatttttgaatg
aatgaatatagatcgaaaaatatagttccattattgttattaacgtgaaacgtagtattggccatgccgcctactcccttat
gactagactgatttctctcataaatacagagatgtacagcttcctttttgtccggagatctaaagataatcttctctcctgtta
ataactctagacgattagtaatatatctcagatcaaagttatgtccgttaaaggtaacgacgtagtcgaacgttagttcca
acaattgtttagctattcgtaacaaaactatttcagaacatagaactagttctcgttcgtaatccatttccattagtgactgta
tcctcaaacatcctctatcgacggcttcttgtatttcctgttccgttaacatctcttcattaatgagcgtaaacaataatcgttt
accacttaaatcgatataacagtaacttgtatgcgagattgggttaataaatacagaaggaaacttcttatcgaagtga
cactctatatctagaaataagtacgatcttgggatatcgaatctaggtatttttttagcgaaacagttacgtggatcgtcac
aatgataacatccattgttaatctttgtcaaatattgctcgtccaacgagtaacatccgtctggagatatcccgttagaaat
ataaaaccaactaatattgagaaattcatccatggtggcattttgtatgctgcgtttctttggctcttctatcaaccacatatc
tgcgacggagcattttctatctttaatatctagattataacttattgtctcgtcaatgtctatagttctcatctttcccaacggcct
cgcattaaatggaggaggagacaatgactgatatatttcgtccgtaactacgtaataaaagtaatgaggaaatcgtat
aaatacggtctcgccatttcgacatctggatttcagatataaaaatctgttttcaccgtgactttcaaaccaattaatgcac
cgaacatccatttatagaatttagaaatatattttcatttaaatgaatcccaaacattggggaagagccgtatggaccatt
atttttatagtactttcgcaagcgggtttagacggcaacatagaagcgtgtaaacgaaaactatatactatagtcagcac
tcttccatgtcctgcatgtagacggcacgcgactatcgctatagaggacaataatgtcatgtctagcgatgatctgaatt
atatttattattttttcatcagattatttaacaatttggcatctgatcccaaatacgcgatcgatgtgacaaaggttaacccttt
ataaacttaacccattataaaacttatgattagtcacgactgaaataaccgcgtgattattttttggtataattctacacggc
atggtttctgtaactatgaattcaacccccgttacattagtgaaatctttaacaaacagcaagggttcgtcaaagacata
aaactcattgtttacaatcgaaatagaccccctatcacacttaaaataaaaaatatccttatcctttaccaccaaataaa
attctgattggtcaatgtgaatgtattcacttaacagttccacaaatttatttattaactccgaggcacatacatcgtcggtat
tttttatggcaaactttactcttccagcatccgtttctaaaaaaatattaacgagttccatttatatcatccaatattattgaaat
gacgttgatggacagatgatacaaataagaaggtacggtacctttgtccaccatctcctccaattcatgctctattttgtca
ttaactttaatgtatgaaaacagtacgccacatgcttccatgacagtgtgtaacactttggatacaaaatgtttgacattag
tataattgtccaagactgtcaatctataatagatagtagctataatatattctatgatggtattgaagaagatgacaatcttg
gcatattgatcatttaacacagacatggtatcaacagatagcttgaatgaaagagaatcagtaattggaataagcgtct
tctcgatagagtgtccgtataccaacatgtctgatattttgatgtattccattaaattatttagttttttctttttattctcgttaaaca
gcatttctgtcaacggaccccaacatcgttgaccgattaagttttgattgatttttccgtgtaaggcgtatctagtcagatcg
tatagcctatccaataatccatcatctgtgcgtagatcacatcgtacactttttaattctctatagaagagcgacagacag
caatttctttattctctacagatgtaagatacttgaagacattcctatgatgatgcagaattttggataacacggtattgatg
gtatctgttaccataattcctttgatggctgatagtgtcagagcacaagatttccaatctttgttttgatatctatatcagacag
catggtgcgtctgacaacacaaggattaagacggaaagatgaaatgattctctcaacatcttcaatggataccttgcta
ttttttctggcattatctatatgtgcgagaatatcctctagagaatcagtatcctttttgatgatagtggatctcaatgacatgg
gacgtctaaaccttcttattctatcaccagattgcatggtgatttgtcttctttcttttatcataatgtaatctctaaattcatcggc
aaattgtctatatctaaaatcataatatgagatgtttacctctacaaatatctgttcgtccaatgttagagtatctacatcagtt
ttgtattccaaattaaacatggcaacggatttaattttatattcctctattaagtcctcgtcgataataacagaatgtagata
atcatttaatccatcgtacatggttggaagatgcttgttgacaaaatctttaattgtcttgatgaaggtgggactatatctaa
catcttgattaataaaatttataacattgtccataggatactttgtaactagttttatacacatctcttcatcggtaagtttagac
agaatatcgtgaacaggtggtatattatattcatcagatatacgaagaacaatgtccaaatctatattgtttaatatattata
tagatgtagcgtagctcctacaggaatatctttaactaagtcaatgatttcatcaaccgttagatctattttaaagttaatcat
ataggcattgatttttaaaaggtatgtagccttgactacattctcattaattaaccattccaagtcactgtgtgtaagaagat
tatattctatcataagcttgactacatttggtcccgataccattaaagaattcttatgatataaggaaacagattttaggtac
tcatctactctacaagaattttggagagccttaacgatatcagtgacgtttattatttcaggaggaaagaatctaacattg
agaatatcggaattaatagcttccagatacagtgattttggcaatagtccgtgtaatccataatccagtaacacgagctg
gtgcttgctagacaccttttcaatgtttaatttttttgaaataagctttgataaagccttcctcgcaaattccggatacatgaa
catgtcggcgacatgattaagtattgttttttcattattttctcaacaagttctcaataccccaatagatgatagaatatcacc
caatgcgtccatgttgtctatttccaacaggtcgctatatccaccaatagaagtttttccaaaaaagattctaggaacagt
tctaccaccagtaatttgttcaaaatagtcacgcaattcattttcgggtttaaattctttaatatcgacaatttcatacgctcct
cttttgaaactaaacttatttagaatatccagtgcatttctacaaaaaggacatgtatacttgacaaaaattgtcactttgtt
attggccaacctttgttgtacaaattcctcggccattttaatatttaagtgatataaaactatctcgacttatttaactctttagt
cgagatatatggacgcagatagctatatgatagccaactacagaaggcaaacgctataaaaaacataattacgacg
agcatatttataaatatttttattcagcattacttgatatagtaatattaggcacagtcaaacattcaaccactctcgatacat
taactctctcattttctttaacaaattctacaatatcttcgtaaaaagattcttgaaactttttagaatatctatcgactctagat
gaaatagcgttcgtcaacatactatgttttgtatacataaaggcgcccattttaacagtttctagtgacaaaatgctagcg
atcctaggatcctttagaatcacatagattgacgattcgtctctcttagtaactctagtaaaataatcatacaatctagtac
gcgaaataatattatccttgacttgaggagatctaaacaatctagttttgagaacatcgataagttcatcgggaatgaca
tacatactatctttaatagaactcttttcatccagttgaatggattcgtccttaaccaactgattaatgagatcttctattttatc
attttccagatgatatgtatgtccattaaagttaaattgtgtagcgcttctttttagtctagcagccaatactttaacatcacta
atatcgatatacaaaggagatgatttatctatggtattaagaattcgtttttcgacatctgtcaaaaccaattcctttttgcctg
tatcatccagttttccatcctttgtaaagaaattattttctactagactattaataagactgataaggattcctccataattgca
caatccaaactttttaacaaaactagactttacaagatctacaggaatgcgtaattcaggtttcttagcttgtgattttttctttt
gtggacattttcttgtgaccaactcatctaccatttcattgattttagcagtgaaataagctttcaatgcacgggcactgata
ctattgaaaacgagttgatcttcaaattccgccatttaagttcaccaaacaacttttaaatacaaatatatcaatagtagta
gaataagaactataaaaaaaataataattaaccaataccaaccccaacaaccggtattattagttgatgtgactgttttc
tcatcacttagaacagatttaacaatttctataaagtctgtcaaatcatcttccggagaccccataaatacaccaaatata
gcggcgtacaacttatccatttatacattgaatattggcttttctttatcgctatcttcatcatattcatcatcaatatcaacaag
tcccagattacgagccagatcttcttctacattttcagtcattgatacacgttcactatctccagagagtccgataacgtta
gccaccacttctctatcaatgattagtttcttgagtgcgaatgtaatttttgtttccgttccggatctatagaagacgataggt
gtgataattgccttggccaattgtctttctcttttactgagtgattctagttcaccttctatagatctgagaatggatgattctcc
agtcgaaacatattctaccatggatccgtttaatttgttgatgaagatggattcatccttaaatgttttctctgtaatagtttcca
ccgaaagactatgcaaagaatttggaatgcgttccttgtgcttaatgtttccatagacggcttctagaagttgatacaaca
taggactagccgcggtaacttttatttttagaaagtatccatcgcttctatcttgtttagatttatttttataaagtttagtctctcct
tccaacataataaaagtggaagtcatttgactagataaactatcagtaagttttatagagatagacgaacaattagcgt
attgagaagcatttagtgtaacgtattcgatacattttgcattagatttactaatcgattttgcatactctataacacccgcac
aagtctgtagagaatcgctagatgcagtaggtcttggtgaagtttcaactctcttcttgattaccttactcatgattaaacct
aaataattgtactttgtaatataatgatatatattttcactttatctcatttgagaataaaaatgtttttgtttaaccactgcatgat
gtacagatttcggaatcgcaaaccaccagtggttttattttatccttgtccaatgtgaattgaatgggagcggatgcgggtt
tcgtacgtagatagtacattcccgtttttagaccgagactccatccgtaaaaatgcatactcgttagtttggaataactcg
gatctgctatatggatattcatagattgactttgatcgatgaaggctcccctgtctgcagccatttttatgatcgtcttttgtgg
aatttcccaaatagttttataaactcgcttaatatcttctggaaggtttgtattctgaatggatccaccatctgccataatcct
attcttgatctcatcattccataattttctctcggttaaaactctaaggagatgcggattaactacttgaaattctccagaca
atactctccgagtgtaaatattactggtatacggttccaccgactcattatttcccaaaatttgagcagttgatgcagtcgg
cataggtgccaccaataaactatttctaagaccgtatgttctgattttatcttttagaggttcccaattccaaagatccgacg
gtacaacattccaaagatcatattgtagaataccgttactggcgtacgatcctacatatgtatcgtatggtccttccttctca
gctagttcacaactcgcctctaatgcaccgtaataaatggtttcgaagatcttcttatttagatcttgtgcttccaggctatca
aatggataatttaagagaataaacgcgtccgctaatccttgaacaccaataccgataggtctatgtctcttattagagatt
tcagcttctggaataggataataattaatatctataattttattgagatttctgacaattactttgaccacatccttcagtttga
gaaaatcaaatcgcccatctattacaaacatgttcaaggcaacagatgccagattacaaacggctacctcattagcat
ccgcatattgtattatctcagtgcaaagattactacacttgatagttcctaaattttgttgattactctttttgttacacgcatcct
tataaagaatgaatggagtaccagtttcaatctgagattctataatcgctttccagacgactcgagcctttattatagatttg
tatctcctttctctttcgtatagtgtatacaatcgttcgaactcgtctccccaaacattgtccaatccaggacattcatccgga
cacatcaacgaccactctccgtcatccttcactcgtttcataaagagatcaggaatccaaagagctataaatagatctc
tggttctatgttcctcgtttcctgtattctttttaagatcgaggaacgccataatatcagaatgccacggttccaagtatatgg
ccataactccaggccgtttgtttcctccctgatctatgtatctagcggtgttattataaactctcaacattggaataataccgt
ttgatataccattggtaccggagatatagcttccactggcacgaatattactaattgatagacctattccccctgccatttta
gagattaatgcgcatcgttttaacgtgtcatagataccctctatgctatcatcgatcatgttaagtagaaaacagctagac
atttggtgacgactagttcccgcattaaataaggtaggagaagcgtgcgtaaaccatttttcagaaagtagattgtacgt
ctcaatagctgagtctatatcccattgatgaattcctactgcgacacgcattaacatgtgctgaggtctttcaacgatcttgt
tgtttattttcaacaagtaggatttttccaaagttttaaaaccaaaatagttgtatgaaaagtctcgttcgtaaataataacc
gagttgagtttatccttatatttgttaactatatccatggtgatacttgaaataatcggagaatgtttcccatttttaggattaac
atagttgaataaatcctccatcacttcactaaatagtttttttgtttccttgtgtagatttgatacggctattctggcggctagaa
tggcataatccggatgttgtgtagtacaagtggctgctatttcggctgccagagtgtccaattctaccgttgttactccatta
tatattccttgaataaccttcatagctattttaataggatctatatgatccgtgtttaagccataacataattttctaatacgag
acgtgattttatcaaacatgacattttccttgtatccatttcgtttaatgacaaacatttttgttggtgtaataaaaaaaattattt
aacttttcattaatagggatttgacgtatgtagcgtacaaaattatcgttcctggtatatagataaagagtcctatatatttga
aaatcgttacggctcgattaaactttaatgattgcatagtgaatatatcattaggatttaactccttgactatcatggcggcg
ccagaaattaccatcaaaagcattaatacagttatgccgatcgcagttagaacggttatagcatccaccatttatatcta
aaaattagatcaaagaatatgtgacaaagtcctagttgtatactgagaattgacgaaacaatgtttcttacatatttttttttt
attagtaaccgacttaatagtaggaactggaaaactagacttgattattctataagtatagatacccttccaaataatatt
ctctttgataaaagttccagaaaatgtagaattttttaaaaagttatcttttgctattaccaagattgtgtttagacgcttattatt
aatatgagtgatgaaatccacaccgcctctagatatcgcttttatttccacattagatggtaaatccaatagtgaaactat
ctttttaggaatgtatggactcgcgtttagaggagtgaacgtcttaggcgtcggaaaggggccggcctttttttctagaca
ctaaataaatagtaagattaaattaattataaaattatgtatataatattaattataaaattatgtatatgatttactaacttta
gttagataaattaataatacataaattttagtatattaatattataaattaataatacataaattttagtatattaatattatatttt
aaatatttatttagtgtctagaaaaaaaggcctagcaggcc

Sequence of sMVA fragment 2 (F2, 63035 bp in length) (deposited with NCBI under Accession No. MW023924, www.ncbi.nlm.nih.gov/nuccore/MW023924.1/) (SEQ ID NO: 13):

ggcctagcaggcctttttttctagacactaaataaatagtaagattaaattaattataaaattatgtatataatattaattata
aaattatgtatatgatttactaactttagttagataaattaataatacataaattttagtatattaatattataaattaataatac
ataaattttagtatattaatattatattttaaatatttatttagtgtctagaaaaaaaggccggccataaaaatgtttttgtttaa
ccactgcatgatgtacagatttcggaatcgcaaaccaccagtggttttattttatccttgtccaatgtgaattgaatgggag
cggatgcgggtttcgtacgtagatagtacattcccgtttttagaccgagactccatccgtaaaaatgcatactcgttagttt
ggaataactcggatctgctatatggatattcatagattgactttgatcgatgaaggctcccctgtctgcagccatttttatga
tcgtcttttgtggaatttcccaaatagttttataaactcgcttaatatcttctggaaggtttgtattctgaatggatccaccatct
gccataatcctattcttgatctcatcattccataattttctctcggttaaaactctaaggagatgcggattaactacttgaaat
tctccagacaatactctccgagtgtaaatattactggtatacggttccaccgactcattatttcccaaaatttgagcagttg
atgcagtcggcataggtgccaccaataaactatttctaagaccgtatgttctgattttatcttttagaggttcccaattccaa
agatccgacggtacaacattccaaagatcatattgtagaataccgttactggcgtacgatcctacatatgtatcgtatgg
tccttccttctcagctagttcacaactcgcctctaatgcaccgtaataaatggtttcgaagatcttcttatttagatcttgtgctt
ccaggctatcaaatggataatttaagagaataaacgcgtccgctaatccttgaacaccaataccgataggtctatgtct
cttattagagatttcagcttctggaataggataataattaatatctataattttattgagatttctgacaattactttgaccacat
ccttcagtttgagaaaatcaaatcgcccatctattacaaacatgttcaaggcaacagatgccagattacaaacggcta
cctcattagcatccgcatattgtattatctcagtgcaaagattactacacttgatagttcctaaattttgttgattactctttttgtt
acacgcatccttataaagaatgaatggagtaccagtttcaatctgagattctataatcgctttccagacgactcgagcctt
tattatagatttgtatctcctttctctttcgtatagtgtatacaatcgttcgaactcgtctccccaaacattgtccaatccagga
cattcatccggacacatcaacgaccactctccgtcatccttcactcgtttcataaagagatcaggaatccaaagagcta
taaatagatctctggttctatgttcctcgtttcctgtattctttttaagatcgaggaacgccataatatcagaatgccacggtt
ccaagtatatggccataactccaggccgtttgtttcctccctgatctatgtatctagcggtgttattataaactctcaacattg
gaataataccgtttgatataccattggtaccggagatatagcttccactggcacgaatattactaattgatagacctattc
cccctgccattttagagattaatgcgcatcgttttaacgtgtcatagataccctctatgctatcatcgatcatgttaagtaga
aaacagctagacatttggtgacgactagttcccgcattaaataaggtaggagaagcgtgcgtaaaccatttttcagaa
agtagattgtacgtctcaatagctgagtctatatcccattgatgaattcctactgcgacacgcattaacatgtgctgaggt
ctttcaacgatcttgttgtttattttcaacaagtaggatttttccaaagttttaaaaccaaaatagttgtatgaaaagtctcgtt
cgtaaataataaccgagttgagtttatccttatatttgttaactatatccatggtgatacttgaaataatcggagaatgtttcc
catttttaggattaacatagttgaataaatcctccatcacttcactaaatagtttttttgtttccttgtgtagatttgatacggctat
tctggcggctagaatggcataatccggatgttgtgtagtacaagtggctgctatttcggctgccagagtgtccaattctac
cgttgttactccattatatattccttgaataaccttcatagctattttaataggatctatatgatccgtgtttaagccataacata
attttctaatacgagacgtgattttatcaaacatgacattttccttgtatccatttcgtttaatgacaaacatttttgttggtgtaa
taaaaaaaattatttaacttttcattaatagggatttgacgtatgtagcgtacaaaattatcgttcctggtatatagataaag
agtcctatatatttgaaaatcgttacggctcgattaaactttaatgattgcatagtgaatatatcattaggatttaactccttg
actatcatggcggcgccagaaattaccatcaaaagcattaatacagttatgccgatcgcagttagaacggttatagca
tccaccatttatatctaaaaattagatcaaagaatatgtgacaaagtcctagttgtatactgagaattgacgaaacaatg
tttcttacatattttttttttattagtaaccgacttaatagtaggaactggaaaactagacttgattattctataagtatagatac
ccttccaaataatattctctttgataaaagttccagaaaatgtagaattttttaaaaagttatcttttgctattaccaagattgt
gtttagacgcttattattaatatgagtgatgaaatccacaccgcctctagatatcgcttttatttccacattagatggtaaatc
caatagtgaaactatctttttaggaatgtatggactcgcgtttagaggagtgaacgtcttaggcgtcggaaaggatgatt
catcaaacgaataaacaatttcacaaatggatgttaatgtattagtaggaaattttttgacgctagtggaattgaagattct
aatggatgatgttctacctatttcatccgataacatgttaatttccgacaccaacggttttaatatttcgatgatatacggtag
tctctctttcggacttatatagcttattccacaatacgagtcattatatactccaaaaaacaaaataactagtataaaatct
gtatcgaatgggaaaaacgaaattatcgacataggtatagaatctggaacattgaacgtattaatacttaattctttttctg
tggtaagtaccgataggttattgacattgtatggttttaaatattctataacttgagacttgatagatattagtgatgaattga
aaattatttttatcaccacgtgtgtttcaggatcatcgtcgacgcccgtcaaccaaccgaatggagtaaaataaatatca
ttaatatatgctctagatattagtatttttatcaatcctttgattatcatcttctcgtaggcgaatgattccatgatcaagagtga
tttaagaacatcctccggagtattaatgggcttagtaaacagtccatcgttgcaataataaaagttatccaagttaaagg
atattatgcattcgtttaaagatatcacctcatctgacggagacaattttttggtaggttttagagactttgaagctacttgttt
aacaaagttattcatcgtcgtttactattctatttaattttgtagttaatttatcacatatcacattaattgactttttggtccatttttc
catacgtttatattcttttaatcctgcgttatccgtttccgttatatccagggatagatcttgcaagttaaatagaatgctcttaa
ataatgtcattttcttatccgctaaaaatttaaagaatgtataaacctttttcagagatttgaaactcttaggtggtgtcctagt
acacaatatcataaacaaactaataaacattccacattcagattccaacagctgattaacttctacattaatacagccta
ttttcgctccaaatgtacattcgaaaaatctgaataaaacatcgatgtcacaatttgtattatccaatacagaatgtttgtga
ttcgtgttaaaaccatcggagaaggaataaaaataaaaattattatagtggtggaattcagttggaatattgcctccgga
gtcataaaaggatactaaacattgttttttatcataaattacacatttccaatgagacaaataacaaaatccaaacattac
aaatctagaggtagaacttttaattttgtctttaagtatatacgataagatatgtttattcataaacgcgtcaaatttttcatga
atcgctaaggagtttaagaatctcatgtcaaattgtcctatataatccacttcggatccataagcaaactgagagactaa
gttcttaatacttcgattgctcatccaggctcctctctcaggctctattttcatcttgacgacctttggattttcaccagtatgtat
tcctttacgtgataaatcatcgattttcaaatccatttgtgagaagtctatcgccttagatactttttcccgtagtcgaggttta
aagaaatacgctaacggtatactagtaggtaactcaaagacatcatatatagaatggtaacgcgtctttaactcgtcgg
ttaactctttcttttgatcgagttcgtcgctactattgggtctgctcaggtgccccgactctactagttccaacatcataccgat
aggaatacaagacactttgccggcggttgtagatttatcatatttctccactacatatccgttacaatttgttaaaaatttag
atacatctatattgctacataatccagctagtgaatatatatgacataataaattggtaaatcctagttctggtattttactaa
ttactaaatctgtatatctttccatttatcatggaaaagaatttaccagatatcttcttttttccaaactgcgttaatgtattctctt
acaaatattcacaagatgaattcagtaatatgagtaaaacggaacgtgatagtttctcattggcggtgtttccagttataa
aacatagatggcataacgcacacgttgtaaaacataaaggaatatacaaagttagtacagaagcacgtggaaaaa
aagtatctcctccatcactaggaaaacccgcacacataaacctaaccgcgaagcaatatatatacagtgaacacac
aataagctttgaatgttatagttttctaaaatgtataacaaatacagaaatcaattcgttcgatgagtatatattaagagga
ctattagaagctggtaatagtttacagatattttccaattccgtaggtaaacgaacagatactataggtgtactagggaat
aagtatccatttagcaaaattccattggcctcattaactcctaaagcacaacgagagatattttcagcgtggatttctcat
agacctgtagttttaactggaggaactggagtgggtaagacgtcacaggtacccaagttattgctttggtttaattatttatt
tggtggattctctactctagataaaatcactgactttcacgaaagaccagtcattctatctcttcctaggatagctttagtta
gattgcatagcaataccattttaaaatcattgggatttaaggtactagatggatctcctatttctttacggtacggatctatac
cggaagaattaataaacaaacaaccaaaaaaatatggaattgtattttctacccataagttatctctaacaaaactattt
agttatggcactcttattatagacgaagttcatgagcatgatcaaataggagatattattatagcagtagcgagaaagc
atcatacgaaaatagattctatgtttttaatgactgccacgttagaggatgacagggaacggctaaaagtatttttaccta
atcccgcatttatacatattcctggagatacactgtttaaaattagcgaggtatttattcataataagataaatccatcttcc
agaatggcatacatagaagaagaaaagagaaatttagttactgctatacagatgtatactcctcctgatggatcatcc
ggtatagtctttgtggcatccgttgcacagtgtcacgaatataaatcatatttagaaaaaagattaccgtatgatatgtata
ttattcatggtaaggtcttagatatagacgaaatattagaaaaagtgtattcatcacctaatgtatcgataattatttctactc
cttatttggaatccagcgttactatacgcaatgttacacacatttatgatatgggtagagtttttgtccccgctccttttggag
gatcgcaagaatttatttctaaatctatgagagatcaacgaaaaggaagagtaggaagagttaatcctgggacatac
gtatatttctatgatctgtcttatatgaagtctatacagcgaatagattcagaatttctacataattatatattgtacgctaata
agtttaatctaacactccccgaagatttgtttataatccctacaaatttggatattctatggcgtacaaaggaatatataga
ctcgttcgatattagtacagaaacatggaataaattattatccaattattatatgaagatgatagagtatgctaaactttatg
tactaagtcctattctcgctgaggagttggataattttgagaggacgggagaattaactagtattgtacaagaagccattt
tatctctaaatttacgaattaagattttaaattttaaacataaagatgatgatacgtatatacacttttgtaaaatattattcgg
tgtctataacggaacaaacgctactatatattatcatagacctctaacgggatatatgaatatgatttcagatactatattt
gttcctgtagataataactaaaaatcaaactctaatgaccacatctttttttagagatgaaaaattttccacatctccttttgt
agacacgactaaacattttgcagaaaaaagtttattagtgtttagataatcgtatacttcatcagtgtagatagtaaatgtg
aacagataaaaggtattcttgctcaatagattggtaaattccatagaatatattaatcctttcttcttgagatcccacatcatt
tcaaccagagacgttttatccaatgatttacctcgtactataccacatacaaaactagattttgcagtgacgtcgtatctgg
tattcctaccaaacaaaattttacttttagttcttttagaaaattctaaggtagaatctctatttgccaatatgtcatctatggaa
ttaccactagcaaaaaatgatagaaatatatattgatacatcgcagctggttttgatctactatactttaaaaacgaatca
gattccataattgcctgtatatcatcagctgaaaaactatgttttacacgtattccttcggcatttctttttaatgatatatcttgtt
tagacaatgataaagttatcatgtccatgagagacgcgtctccgtatcgtataaatatttcattagatgttagacgcttcatt
aggggtatacttctataaggtttcttaatcagtccatcattggttgcgtcaagaactactatcggatgttgttgggtatctcta
gtgttacacatggccttactaaagtttgggtaaataactatgatatctctattaattatagatgcatatatttcatttgtcaagg
atattagtatcgacttgctatcgtcattaatacgtgtaatgtaatcatataaatcatgcgatagccaaggaaaatttaaata
gatgttcatcatataatcgtcgctataattcatattaatacgttgacattgactaatttgtaatatagcctcgccacgaagaa
agctctcgtattcagtttcatcgataaaggataccgttaaatataactggttgccgatagtctcatagtctattaagtggtaa
gtttcgtacaaatacagaatccctaaaatattatctaatgttggattaatctttaccataactgtataaaatggagacgga
gtcataactattttaccgtttgtacttactggaatagacgaaggaataatctccggacatgctggtaaagacccaaatgt
ctgtttgaagaaatccaatgttccaggtcctaatctcttaacaaaaattacgatattcgatcccgatatcctttgcattctattt
accagcatatcacgaactatattaagattatctatcatgtctattctcccaccgttatataaatcgcctccgctaagaaac
gttagtatatccatacaatggaatacttcatttctaaaatagtattcgttttctaattctttaatgtgaaatcgtatactagaaa
gggaaaaattatctttgagttttccgttagaaaagaaccacgaaactaatgttctgattgcgtccgattccgttgctgaatt
aatggatttacaccaaaaactcatataacttctagatgtagaagcattcgctaaaaaattagtagaatcaaaggatata
agtagatgttccaacaagtgagcaattcccaagatttcatctatatcattctcgaatccgaaattagaaattcccaagta
gatatcctttttcatccgatcgttgatgaaaatacgaactttattcggtaagacaatcatttactaaggagtaaaatagga
agtaatgttcgtatgtcgttatcatcgtataaattaaaggtgtgttttttaccattaagtgacattataattttaccaatattgga
attataatataggtgtatttgcgcactcgcgacggttgatgcatcggtaaatatagctgtatctaatgttctagtcggtatttc
atcatttcgctgtctaataatagcgttttctctatctgtttccattacagctgcctgaagtttattggtcggataatatgtaaaat
aataagaaatacatacgaataacaaaaataaaataagatataataaagatgccatttagagatctaattttgtttaactt
gtccaaattcctacttacagaagatgaggaatcgttggagatagtgtcttccttatgtagaggatttgaaatatcttatgat
gacttgataacttactttccagataggaaataccataaatatatttctaaagtatttgaacatgtagatttatcggaggaatt
aagtatggaattccatgatacaactctgagagatttagtctatcttagattgtacaagtattccaagtgtatacggccgtgt
tataaattaggagataatctaaaaggcatagttgttataaaggacaggaatatttatattagagaagcaaatgatgactt
gatagaatatctcctcaaggaatacactcctcagatttatacatattctaatgagcgcgtccccataactggttcaaaatt
aattctttgtggattttctcaagttacatttatggcgtatacaacgtcgcatataacaacaaataaaaaggtagatgttctc
gtttccaaaaaatgtatagatgaactagtcgatccaataaattatcaaatacttcaaaatttatttgataaaggaagcgg
aacaataaacaaaatactcaggaagatattttattcggtaaccggtggccaaactccataatttgctttttctatttcggatt
ttagaatttccaaattcaccagcgatttatcggttttggtgaaatccaaggatttattaatgtccacaaatgccatttgttttgt
ctgtggattgtatttgaaaatggaaacgatgtagttagatagatgcgctgcaaagtttcctattagggttccgcgctttacg
tcacccagcatacttgaatcaccatcctttaaaaaaaatgataagatatcaacatggagtatatcatactcggattttaat
tcttctactgcatcactgacattttcacaaatactacaatacggtttaccgaaaataatcagtacgttcttcatttatgggtat
caaaaacttaaaatcgttactgctggaaaataaatcactgacgatattagatgataatttatacaaagtatacaatgga
atatttgtggatacaatgagtatttatatagccgtcgccaattgtgtcagaaacttagaagagttaactacggtattcataa
aatacgtaaacggatgggtaaaaaagggagggcatgtaaccctttttatcgatagaggaagtataaaaattaaaca
agacgttagagacaagagacgtaaatattctaaattaaccaaggacagaaaaatgctagaattagaaaagtgtaca
tccgaaatacaaaatgttaccggatttatggaagaagaaataaaggcagaaatgcaattaaaaatcgataaactca
catttcaaatatatttatctgattctgataacataaaaatatcattgaatgagatactaacacatttcaacaataatgagaa
tgttacattattttattgtgatgaacgagacgcagaattcgttatgtgtctcgaggctaaaacacatttctctaccacagga
gaatggccgttgataataagtaccgatcaggatactatgctatttgcatctactgataatcatcctaagatgataaaaaa
cttaactcaactgtttaaatttgttccctcggcagaggataactatttagcaaaattaacggcgttagtgaatggatgtgat
ttctttcctggactctatggggcatctataacacccaccaacttaaacaaaatacaattgtttagtgattttacaatcgata
atatagtcactagtttggcaattaaaaattattatagaaagactaactctaccgtagacgtgcgtaatattgttacgtttata
aacgattacgctaatttagacgatgtctactcgtatgttcctccttgtcaatgcactgttcaagaatttatattttccg cattag
atgaaaaatggaacaattttaaatcatcttatttagagaccgttccgttaccctgccaattaatgtatgcattagaaccac
gcaaggagattgatgtttcagaagttaaaactttatcatcttatatagatttcgaaaatactaaatcagatatcgatgttata
aaatctatatcttcgatcttcggatattctaacgaaaactgtaacactatagtgttcggcatctataaggataatttactact
gagtataaatagttcattttactttaacgatagtctgttaataaccaatactaaaagtgataatataataaatataggttact
agattaaaaatggtgttccaactcgtgtgctctacgtgcggcaaagatatttctcacgaacgatataaattgattatacga
aaaaaatcattaaaggatgtactcgtcagtgtaaagaacgaatgttgtaggttaaaattatctacacaaatagaacctc
aacgtaacttaacagtgcaacctctattggatataaactaatatggatccggttaattttatcaagacatatgcgcctaga
ggttctattatttttattaattataccatgtcattaacaagtcatttgaatccatcgatagaaaaacatgtgggtatttattatgg
tacgttattatcggaacacttggtagttgaatctacctatagaaaaggagttcgaatagtcccattggatagtttttttgaag
gatatcttagtgcaaaagtatacatgttagagaatattcaagttatgaaaatagcagctgatacgtcattaactttattggg
tattccgtatggatttggtcataatagaatgtattgttttaaattggtagctgaatgttataaaaatgccggtattgatacatc
gtctaaacgaatattaggtaaagatatttttctgagccaaaacttcacagatgataatagatggataaagatatatgattc
taataatttaacattttggcaaattgattaccttaaagggtgagttaatatgcataactactcctccgttgttttttccctcgttct
ttttcttaacgttgtttgccatcactctcataatgtaaagatattctaaaatggtaaacttttgcatatcggacgcagaaattg
gtataaatgttgtaattgtattatttcccgtcaatggactagtcacagctccatcagttttatatcctttagagtatttctcactc
gtgtctaacattctagagcattccatgatctgtttatcgttgatattggccggaaagatagattttttattttttattatattactatt
ggcaattgtagatataacttctggtaaatatttttctaccttttcaatctcttctattttcaagccggctatatattctgctatattgt
tgctagtatcaataccttttctggctaagaagtcatatgtggtattcactatatcagttttaactggtagttccattagcctttcc
acttctgcagaataatcagaaattggttctttaccagaaaatccagctactataataggctcaccgatgatcattggcaa
aatcctatattgtaccagattaatgagagcatatttcatttccaataattctgctagttcttgagacattgatttatttgatgaat
ctagttggttctctagatactctaccatttctgccgcatacaataacttgttagataaaatcagggttatcaaagtgtttagc
gtggctagaatagtgggcttgcatgtattaaagaatgcggtagtatgagtaaaccgttttaacgaattatatagtctccag
aaatctgtggcgttacatacatgagccgaatgacatcgaagattgtccaatatttttaatagctgctctttgtccattatttct
atatttgactcgcaacaattgtagataccattaatcactgattcctttttcgatgccggacaatagcacaattgtttagctttg
gactctatgtattcagaattaatagatatatctctcaatacagattgcactatacattttgaaactatgtcaaaaattgtaga
acgacgctgttctgcagccatttaactttaaataatttacaaaaatttaaaatgagcatccgtataaaaatcgataaactg
cgccaaattgtggcatatttttcagagttcagtgaagaagtatctataaatgtagactcgacggatgagttaatgtatatttt
tgccgccttgggcggatctgtaaacatttgggccattatacctctcagtgcatcagtgttctaccgcggagccgaaaac
attgtgtttaatcttcctgtgtccaaggtaaaatcgtgtttgtgtagttttcacaatgatgccatcatagatatagaacctgatc
tggaaaataatctagtaaaactttctagttatcatgtagtaagtgtcgattgtaacaaggaactgatgcctattaggacag
atactactatttgtctaagtatagatcaaaagaaatcttacgtgtttaattttcacaagtatgaagaaaaatgttgtggtag
aaccgtcattcatttagaatggttgttgggctttatcaagtgtattagtcagcatcagcatttggctattatgtttaaagatga
caatattattatgaagactcctggtaatactgatgcgttttccagggaatattctatgactgaatgttctcaagaactacaa
aagttttctttcaaaatagctatctcgtctctcaacaaactacgaggattcaaaaagagagtcaatgtttttgaaactaga
atcgtaatggataatgacgataacattctaggaatgttgttttcggatagagttcaatcctttaagatcaacatctttatgac
gtttttagattaatactttcaatgagataaatatgggtggcagagtaagtgttgagctccctaaacgggatccgcctccgg
gagtacccactgatgagatgttattaaacgtggataaaatgcatgacgtgatagctcccgctaagcttttagaatatgtg
catataggaccactagcaaaagataaagaggataaagtaaagaaaagatatccagagtttagattagtcaacaca
ggacccggtggtctttcggcattgttaagacaatcgtataatggaaccgcacccaattgctgtcgcacttttaatcgtact
cattattggaaaaaggatggaaagatatcagataagtatgaagagggtgcagtattagaatcgtgttggccagacgtt
cacgacactggaaaatgcgatgttgatttattcgactggtgtcagggggatacgttcgatagaaacatatgccatcagt
ggatcggttcagcctttaataggagtgatagaactgtagagggtcaacaatcgttaataaatctgtataataagatgca
aacattatgtagtaaagatgctagtgtaccaatatgtgaatcatttttgcatcatttacgcgcacacaatacagaagata
gcaaagagatgatcgattatattctaagacaacagtctgcggactttaaacagaaatatatgagatgtagttatcccac
tagagataagttagaagagtcattaaaatatgcggaacctcgagaatgttgggatccagagtgttcgaatgccaatgtt
aatttcttactaacacgtaattataataatttaggactttgcaatattgtacgatgtaatactagcgtgaacaacttacagat
ggataaaacttcctcattaagattgtcatgtggattaagcaatagtgatagattttctactgttcccgtcaatagagcaaa
agtagttcaacataatattaaacattcgttcgacctaaaattgcatttgatcagtttattatctctcttggtaatatggatacta
attgtagctatttaaatgggtgccgcggcaagcatacagacgacggtgaatacactcagcgaacgtatctcgtctaaa
ttagaacaagaagcgaacgctagtgctcaaacaaaatgtgatatagaaatcggaaatttttatatccgacaaaacca
tggatgtaacctcactgttaaaaatatgtgctctgcggacgcggatgctcagttggatgctgtgttatcagccgctacag
aaacatatagtggattaacaccggaacaaaaagcatacgtaccagctatgtttactgctgcgttaaacattcagacga
gtgtaaacactgttgttagagattttgaaaattatgtgaaacagacttgtaattctagcgcggtcgtcgataacaaattaa
agatacaaaacgtaatcatagatgaatgttacggagccccaggatctccaacaaatttggaatttattaatacaggatc
tagcaaaggaaattgtgccattaaagcgttgatgcaattgacgactaaggccactactcaaatagcacctagacaag
ttgctggtacaggagttcagttttatatgattgttatcggtgttataatattggcagcgttgtttatgtactatgccaagcgtatg
ttgttcacatccaccaatgataaaatcaaacttattttagccaataaggaaaacgtccattggactacttacatggacac
attctttagaacttctccgatggttattgctaccacggatatgcaaaactgaaaatatattgataatattttaatagattaac
atggaagttatcgctgatcgtctagacgatatagtgaaacaaaatatagcggatgaaaaatttgtagattttgttatacac
ggtctagagcatcaatgtcctgctatacttcgaccattaattaggttgtttattgatatactattatttgttatagtaatttatatttt
tacggtacgtctagtaagtagaaattatcaaatgttgttggcgttggtggcgctagtcatcacattaactattttttattacttt
atactataatagtactagactgacttctaacaaacatctcacctgccataaataaatgcttgatattaaagtcttctatttct
aacactattccatctgtggaaaataatactctgacattatcgctaattgacacatcggtgagtgatatgcctataaagtaa
taatcttctttgggcacatataccagtgtaccaggttctaacaacctatttactggtgctcctgtagcatactttttctttacctt
gagaatatccatcgtttgcttggtcaatagcgatatgtgattttttatcaaccactcaaaaaagtaattggagtgttcatatc
ctctacgggctattgtctcatggccgtgtatgaaatttaagtaacacgactgtggtagatttgttctatagagccggttgcc
gcaaatagatagaactaccaatatgtctgtacaaatgttaaacattaattgattaacagaaaaaacaatgttcgttctgg
gaatagaaaccagatcaaaacaaaattcgttagaatatatgccacgtttatacatggaatataaaataactacagtttg
aaaaataacagtatcatttaaacatttaacttgcggggttaatttcacaactttactgtttttaaactgttcaaaatatagcat
cgatccatgagaaatacgtttagccgcctttaatagaggaaatcccaccgcctttctggatctcaccaacgacgatagt
tctgaccagcaacttatttcttcatcatccacctgttttaacatataataggcaggagatagatatccgtcattgcaatattc
cttttcgtaggcacacaatctaatattgataaaatctccattctcttctctgcatttattatcttgtttcggtggctgattaggctg
tagtcttggtttaggctttggtatatcgttgttgaatctattttggtcattaaatctttcatttcttcctggtatatttctatcacctcgtt
tggttggatttttgtctatattatcgtttgtaacatcggtacgggtattcatttatcacaaaaaaaacttctctaaatgagtcta
ctgctagaaaacctcatcgaagaagataccatattttttgcaggaagtatatctgagtatgatgatttacaaatggttattg
ccggcgcaaaatccaaatttccaagatctatgctttctatttttaatatagtacctagaacgatgtcaaaatatgagttgga
gttgattcataacgagaatatcacaggggcaatgtttaccacaatgtataatataagaaacaatttgggtctaggagat
gataaactaactattgaagccattgaaaactatttcttggatcctaacaatgaggttatgcctcttatcattaataatacgg
atatgactgccgtcattcctaaaaaaagtggtaggagaaagaataagaacatggttattttccgtcaaggatcatcacc
tatcttgtgtattttcgaaactcgtaaaaagattaatatttataaagaaaatatggaatccgcgtcgactgagtatacacct
atcggagacaacaaggctttgatatctaaatatgcgggaattaatgtcctgaatgtgtattctccttccacatccatgaga
ttgaatgccatttacggattcaccaataaaaataaactagagaaacttagtactaataaggaactagaatcgtatagtt
ctagccctcttcaagaacccattaggttaaatgattttctgggactattggaatgtgttaaaaagaatattcctctaacaga
tattccgacaaaggattgattactataaatggagaatgttcctaatgtatactttaatcctgtgtttatagagcccacgttta
aacattctttattaagtgtttataaacacagattaatagttttatttgaagtattcattgtattcattctaatatatgtattttttagat
ctgaattaaatatgttcttcatgcctaaacgaaaaatacccgatcctattgatagattacgacgtgctaatctagcgtgtg
aagacgataaattaatgatctatggattaccatggatgacaactcaaacatctgcgttatcaataaatagtaaaccgat
agtgtataaagattgtgcaaagcttttgcgatcaataaatggatcacaaccagtatctcttaacgatgttcttcgcagatg
atgattcattttttaagtatttggctagtcaagatgatgaatcttcattatctgatatattgcaaatcactcaatatctagacttt
ctgttattattattgatccaatcaaaaaataaattagaagccgtgggtcattgttatgaatctctttcagaggaatacagac
aattgacaaaattcacagactttcaagattttaaaaaactgtttaacaaggtccctattgttacagatggaagggtcaaa
cttaataaaggatatttgttcgactttgtgattagtttgatgcgattcaaaaaagaatcctctctagctaccaccgcaatag
atcctattagatacatagatcctcgtcgcgatatcgcattttctaacgtgatggatatattaaagtcgaataaagtgaaca
ataattaattctttattgtcatcatgaacggcggacatattcagttgataatcggccccatgttttcaggtaaaagtacaga
attaattagacgagttagacgttatcaaatagctcaatataaatgcgtgactataaaatattctaacgataatagatacg
gaacgggactatggacgcatgataagaataattttgaagcattggaagcaactaaactatgtgatgtcttggaatcaat
tacagatttctccgtgataggtatcgatgaaggacagttctttccagacattgttgaattctgtgagcgtatggcaaacga
aggaaaaatagttatagtagccgcactcgatgggacatttcaacgtaaaccgtttaataatattttgaatcttattccatta
tctgaaatggtggtaaaactaactgctgtgtgtatgaaatgctttaaggaggcttccttttctaaacgattgggtgaggaa
accgagatagagataataggaggtaatgatatgtatcaatcggtgtgtagaaagtgttacgtcggctcataatattatat
tttttatctaaaaaactaaaaataaacattgattaaattttaatataatacttaaaaatggatgttgtgtcgttagataaacc
gtttatgtattttgaggaaattgataatgagttagattacgaaccagaaagtgcaaatgaggtcgcaaaaaaactgccg
tatcaaggacagttaaaactattactaggagaattattttttcttagtaagttacagcgacacggtatattagatggtgcca
ccgtagtgtatataggatctgctcccggtacacatatacgttatttgagagatcatttctataatttaggagtgatcatcaaa
tggatgctaattgacggccgccatcatgatcctattttaaatggattgcgtgatgtgactctagtgactcggttcgttgatga
ggaatatctacgatccatcaaaaaacaactgcatccttctaagattattttaatttctgatgtgagatccaaacgaggag
gaaatgaacctagtacggcggatttactaagtaattacgctctacaaaatgtcatgattagtattttaaaccccgtggcgt
ctagtcttaaatggagatgcccgtttccagatcaatggatcaaggacttttatatcccacacggtaataaaatgttacaa
ccttttgctccttcatattcagctgaaatgagattattaagtatttataccggtgagaacatgagactgactcgagttacca
aatcagacgctgtaaattatgaaaaaaagatgtactaccttaataagatcgtccgtaacaaagtagttgttaactttgatt
atcctaatcaggaatatgactattttcacatgtactttatgctgaggaccgtgtactgcaataaaacatttcctactactaa
agcaaaggtactatttctacaacaatctatatttcgtttcttaaatattccaacaacatcaactgaaaaagttagtcatgaa
ccaatacaacgtaaaatatctagcaaaaattctatgtctaaaaacagaaatagcaagagatccgtacgcagtaataa
atagaaacgtactactgagatatactaccgatatagagtataatgatttagttactttaataaccgttagacataaaattg
attctatgaaaactgtgtttcaggtatttaacgaatcatccataaattatactccggttgatgatgattatggagaaccaat
cattataacatcgtatcttcaaaaaggtcataacaagtttcctgtaaattttctatacatagatgtggtaatatctgacttattt
cctagctttgttagactagatactacagaaactaatatagttaatagtgtactacaaacaggcgatggtaaaaagactc
ttcgtcttcccaaaatgttagagacggaaatagttgtcaagattctctaccgtcctaatataccattaaaaattgttagatttt
tccgcaataacatggtaactggagtagagatagccgatagatctgttatttcagtcgctgattaatcaattagtagagat
gagataagaacattataataatcaataatatattttatatcttatatcttgtttagaaaaatgctaatattaaaatagctaac
gctagtaatccaatcggaagccatttgatatctataatagggtatctaatttcctgattcagatagcggacagctatattct
cggtagctactcgtttggaatcacaaacattatttacatctaatttactatctgtaatggaaacgtttcccaatgaaatggta
caatccgatacattgcattttgttatatttttttttaaagaggctggtaacaacgcatcgcttcgtttacatggctcgtaccaa
caataatagggtaatcttgtatctattcctatccgtactatgcttttatcaggataaatacatttacatcgtatatcgtctttgtta
gcatcacagaatgcataaatttgttcgtccgtcatgataaaaatttaaagtgtaaatataactattattttatagttgtaataa
aaagggaaatttgattgtatactttcggttctttaaaagaaactgacttgataaaaatggctgtaatctctaaggttacgtat
agtctatatgatcaaaaagagattaatgctacagatattatcattagtcatgttaaaaatgacgacgatatcggtaccgtt
aaagatggtagactaggtgctatggatggggcattatgtaaaacttgtgggaaaacggaattggaatgtttcggtcact
ggggtaaagtaagtatttataaaactcatatagttaagcctgaatttatttcagaaattattcgtttactgaattatatatgtat
tcactgcggattattgcgttcacgagaaccgtattccgacgatattaacctaaaagagttatcgggacacgctcttagg
agattaaaggataaaatattatccaagaaaaagtcatgttggaacagtgaatgtatgcaaccgtatcaaaaaattactt
tttcaaagaaaaaggtttgtttcgtcaacaagttggatgatattaacgttcctaattctctcatctatcaaaagttaatttctat
tcatgaaaagttttggccattattagaaattcatcaatatccagctaacttattttatacagactactttcccatccctccgttg
attattagaccggctattagtttttggatagatagtatacccaaagaaaccaatgaattaacttacttattaggtatgatcgt
taagaattgtaacttgaatgctgatgaacaggttatccagaaggcggtaatagaatacgatgatattaaaattatttcta
ataacacttccagtatcaatttatcatatatcacatccggcaaaaataatatgattagaagttatatcgtcgcccggcga
aaagatcagaccgctagatctgtaattggtcccagtacatctatcaccgttaatgaggtaggaatgcccgcatatatta
gaaatacacttacagaaaagatatttgttaatgcctttacagtggataaagttaaacaactattagcgtcaaaccaagtt
aaattttactttaataaacgattaaaccaattaacaagaatacgccaaggaaagtttatcaaaaataaaatacatttatt
gcctggtgattgggtagaagtagctgttcaagaatatacaagtattatttttggaagacagccgtctctacatagataca
acgtcatcgcttcatctatcagagctaccgaaggagatactatcaaaatatctcccggaattgtcaactctcaaaatgct
gatttcgacggagatgaagaatggatgatattggagcaaaatcctaaagccgtaattgaacaaagtattcttatgtatc
cgacgacgttactcaaacacgatattcatggagcccccgtttatggatctattcaagatgaaatcgtagcagcgtattca
ttgtttaggatacaagatctttgtttagatgaagtattgaacatcttggggaaatatggaagagagttcgatcctaaaggt
aaatgtaaattcagcggtaaagatatctatacttacttgataggtgaaaagattaattatccgggtctcttaaaggatggt
gaaattattgcaaacgacgtagatagtaattttgttgtggctatgaggcatctgtcattggctggactcttatccgatcata
agtcgaacgtggaaggtatcaactttattatcaagtcatcttatgtttttaagagatatctatctatttacggttttggggtgac
attcaaagatctgagaccaaattcgacgttcactaataaattggaggccatcaacgtagaaaaaatagaacttatcaa
agaagcatacgccaaatatctcaacgatgtaagagacgggaaaatagttccattatctaaagctttagaggcggact
atgtggaatccatgttatccaacttgacaaatcttaatatccgagagatagaagaacatatgagacaaacgctgatag
atgatccagataataacctcctgaaaatggccaaagcgggttataaagtaaatcccacagaactaatgtatattctag
gtacttatggacaacagaggattgatggtgaaccagcagagactcgagtattgggtagagtcttaccttactatcttcca
gactctaaggatccagaaggaagaggttacattcttaattctttaacaaaaggattaacgggttctcaatattacttttcg
atgctggttgcaagatctcaatctactgatatcgtctgtgaaacatcacgtaccggaacactggctagaaaaatcatta
aaaagatggaggatatggtggtcgacggatacggacaagtagttataggtaatacgctcatcaagtacgccgccaa
ttataccaaaattctaggctcagtatgtaaacctgtagatcttatctatccagatgagtccatgacttggtatttggaaatta
gtgctctgtggaataaaataaaacagggattcgtttactctcagaaacagaaacttgcaaaaaagacattggcgccgt
ttaatttcctagtattcgtcaaacccaccactgaggataatgctattaaggttaaggatctgtacgatatgattcataacgt
cattgatgatgtgagagagaaatacttctttacggtatctaatatagattttatggagtatatattcttgacgcatcttaatcct
tctagaattagaattacaaaagaaacggctatcactatctttgaaaagttctatgaaaaactcaattatactctaggtggt
ggaactcctattggaattatttctgcacaggtattgtctgagaagtttacacaacaagccctgtccagttttcacactactg
aaaaaagtggtgccgtcaaacaaaaacttggtttcaacgagtttaataacttgactaatttgagtaagaataagaccg
aaattatcactctggtatccgatgatatctctaaacttcaatctgttaagattaatttcgaatttgtatgtttgggagaattaaa
tccaaacatcactcttcgaaaagaaacagataggtatgtagtagatataatagtcaatagattatacatcaagagagc
agaaattaccgaattagtcgtcgaatatatgattgaacgattcatctcctttagcgtcattgtaaaggaatggggtatgga
aacattcattgaggatgaggataatattagatttactgtctacctaaatttcgttgaaccggaagaattgaatcttagtaag
tttatgatggttcttccgggtgccgccaacaagggcaagattagtaaattcaagattcctatctctgattatacgggatatg
acgacttcaatcaaacaaaaaagctcaataagatgactgtagaactcatgaatctaaaagaattgggttctttcgattt
ggaaaacgtcaacgtgtatcctggagtatggaatacatacgatatcttcggtatcgaggccgctcgtgaatacttgtgc
gaagccatgttaaacacctatggagaagggttcgattatctgtatcagccttgtgatcttctcgctagtttactatgtgctag
ttacgaaccagaatcagtgaataaattcaagttcggcgcagctagtactcttaagagagctacgttcggagacaataa
agcattgttaaacgcggctcttcataaaaagtcagaacctattaacgataatagtagctgccacttttttagcaaggtcc
ctaatataggaactggatattacaaatactttatcgacttgggtcttctcatgagaatggaaaggaaactatctgataag
atatcttctcaaaagatcaaggaaatggaagaaacagaagacttttaattcttatcaataacatatttttctatgatctgtct
tttaaacgatggattttccacaaatgcgcctctcaagtccctcatagaatgatacacgtataaaaaatatagcataggc
aatgactccttatttttagacattagatatgccaaaatcatagccccgcttctatttactcccgcagcacaatgaaccaac
acgggctcgtttcgttgatcacatttagataaaaaggcggttacgtcgtcaaaatatttactaatatcggtagttgtatcatc
taccaacggtatatgaataatattaatattagagttaggtaatgtatatttatccatcgtcaaatttaaaacatatttgaactt
aacttcagatgatggtgcatccatagcatttttataatttcccaaatacacattattggttactcttgtcattatagtgggagat
ttggctttgtgcatatctccagttgaacgtagtagtaagtatttatacaaacttttcttatccatttataacgtacaaatggata
aaactactttatcggtaaacgcgtgtaatttagaatacgttagagaaaaggctatagtaggcgtacaagcagccaaa
acatcaacacttatattctttgttattatattggcaattagtgcgctattactctggtttcagacgtctgataatccagtctttaat
gaattaacgagatatatgcgaattaaaaatacggttaacgattggaaatcattaacggatagcaaaacaaaattaga
aagtgatagaggtagacttctagccgctggtaaggatgatatattcgaattcaaatgtgtggatttcggcgcctattttata
gctatgcgattggataagaaaacatatctgccgcaagctattaggcgaggtactggagacgcgtggatggttaaaaa
ggcggcaaaggtcgatccatctgctcaacaattttgtcagtatttgataaaacacaagtctaataatgttattacttgtggt
aatgagatgttaaatgaattaggttatagcggttattttatgtcaccgcattggtgttccgattttagtaatatggaatagtgtt
agataaatgcggtaacgaatgttcctgtaaggaaccataacagcttagatttaacgttaaagatgagcataaacataa
taaacaaaattacaatcaaacctataacattaatatcaaacaatccaaaaaatgaaatcagtggagtagtaaacgc
gtacataactcctggataacgtttagcagctgccgttcctattctagaccaaaaattcggtttcatgttttcgaaacggtatt
ctgcaacaagtcgaggatcgtgttctacatatttggcggcgttatccagtatctgcctattgatcttcatttcgttttcgattctg
gctatttcaaaataaaatcccgatgatagacctccagactttataatttcatctacgatgttcagcgccgtagtaactctaa
taatataggctgataagctaacatcataccctcctgtatatgtgaatatggcatgatttttgtccattacaagctcggttttaa
ctttattgcctgtaataatttctctcatctgtaggatatctatttttttgtcatgcattgccttcaagacgggacgaagaaacgt
aatatcctcaataacgttatcgttttctacaataactacatattctacctttttattttctaactcggtaaaaaaattagaatcc
catagggctaaatgtctagcgatatttcttttcgtttcctctgtacacatagtgttacaaaaccctgaaaagaagtgagtat
acttgtcatcatttctaatgtttcctccagtccactgtataaacgcataatccttgtaatgatctggatcatccttgactacca
caacatttcttttttctggcataacttcattgtcctttacatcatcgaacttctgatcattaatatgctcatgaacattaggaaat
gtttctgatggaggtctatcaataactggcacaacaataacaggagttttcaccgccgccatttagttattgaaattaatc
atatacaactctttaatacgagttatattttcgtctatccattgtttcacatttacatatttcgacaaaaagatataaaatgcgt
attccaatgcttctctgtttaatgaattactaaaatatacaaacacgtcactgtctggcaataaatgatatcttagaatattgt
aacaatttattttgtattgcacatgttcgtgatctatgagttcttcttcgaatggcataggatctccgaatctgaaaacgtata
aataggagttagaataataatatttgagagtattggtaatatataaactctttagcggtataattagtttttttctctcgatttct
atttttagatgtgatggaaaaatgactaattttgtagcattagtatcatgaactctaatcgagatcttaatatcttcgtcacac
gttagttctttgaagtttttaagagatgcatcagttggttcgaccgatggagtaggtgcaacaattttttgttcgatgtatgtat
gtactggagccattgtcttaactataatggtgcttgtatcgaaaaactttaatgcagataatggaagctcttcgccgcgac
tttctacatcgtaattgggttctaacgccgatctctgaatggatactagttttctaagttctaatgtgattctctgaaaatgtaa
atccaattcctccggcattatagatgtgtatacatcggtaaataaaactatagtatccaacgatcccttctcgcaaattcta
gtcttaaccaaaaaatcgtatataaccacggagatggcgtatttaagagtggattcttctaccgttttgttcttggatttcata
taagaaactataaagtccgcactactgttaagaatgattactaacgcaactatatagtttaaattaagcatcttggaaac
ataaaataactctgtagacgatacttgactttcgaataagtttgcagacaaacgaagaaagaacagacctctcttaattt
cagaagaaaactttttttcgtattcctgacgtctagagtttatatcaataagaaagttaagaattagtcggttaatgttgtattt
cattacccaagtttgagatttcataatattatcaaaagacatgataatattaaagataaagcgctgactatgaacgaaat
agctatatggttcgctcaagaatatagtcttgttaaacgtggaaacgataactgtatttttaatcacgtcagcggcatctaa
attaaatataggtatatttattccacacactctacaatatgccacaccatcttcataataaataaattcgttagcaaaattat
taattttagtgaaatagttagcgtcaactttcatagcttccttcaatctaatttgatgctcacacggtgcgaattccactctaa
catcccttttccatgcctcaggttcatcgatctctataatatctagttttttgcgtttcacaaacacaggctcgtctctcgcgat
gagatctgtatagtaactatgtaaatgataactagatagaaagatgtagctatatagatgacgatcctttaagagaggt
atgatgactttaccccaatcagatagactgttgttatggtcttcggaaaaagaatttttataaatttttccagtattttccaaat
atacgtacttaacatctaaaaaatccttaatgataataggaatggataatccgtctattttataaagaaatacatatcgca
cattatacttttttttggaaatgggaataccgatgtgtctacataaatatgcaaagtctaaatattttttagagaatcttagttg
gtccaaattcttttccaagtacggtaatagatttttcatattgaacggtatcttcttaatctctggttctagttccgcattaaatg
atgaaactaagtcactatttttataactaacgattacatcacctctaacatcatcatttaccagaatactgatcttcttttgtcg
taaatacatgtctaatgtgttaaaaaaaagatcatacaagttatacgtcatttcatctgtggtattcttgtcattgaaggata
aactcgtactaatctcttctttaacagcctgttcaaatttatatcctatatacgaaaaaatagcaaccagtgtttgatcatcc
gcgtcaatattctgttctatcgtagtgtataacaatcgtatatcttcttctgtgatagtcgatacgttataaaggttgataacg
aaaatatttttatttcgtgagataaagtcatcgtaggattttggacttatattcgcgtctagtagatatgcttttatttttggaatg
atctcaattagaatagtctctttagagtccatttaaagttacaaacaactaggaaattggtttatgatgtataatttttttagtttt
tatagattctttattctatacttaaaaaatgaaaataaatacaaaggttcttgagggttgtgttaaattgaaagcgagaaat
aatcataaattatttcattatcgcgatatccgttaagtttgtatcgtaatggcgtggtcaattacaaataaagcggatacta
gtagcttcacaaagatggctgaaatcagagctcatctaaaaaatagcgctgaaaataaagataaaaacgaggatat
tttcccggaagatgtaataattccatctactaagcccaaaaccaaacgagccactactcctcgtaaaccagcggctac
taaaagatcaaccaaaaaggaggaagtggaagaagaagtagttatagaggaatatcatcaaacaactgaaaaa
aattctccatctcctggagtcagcgacattgtagaaagcgtggctgctgtagagctcgatgatagcgacggggatgat
gaacctatggtacaagttgaagctggtaaagtaaatcatagtgctagaagcgatctttctgacctaaaggtggctaccg
acaatatcgttaaagatcttaagaaaattattactagaatctctgcagtatcgacggttctagaggatgttcaagcagct
ggtatctctagacaatttacttctatgactaaagctattacaacactatctgatctagtcaccgagggaaaatctaaagtt
gttcgtaaaaaagttaaaacttgtaagaagtaaatgcgtgcacttttttataaagatggtaaactctttaccgataataattt
tttaaatcctgtatcagacgataatccagcgtatgaggttttgcaacatgttaaaattcctactcatttaacagatgtagtag
tatatgaacaaacgtgggaggaggcgttaactagattaatttttgtgggaagtgattcaaaaggacgtagacaatactt
ttacggaaaaatgcatgtacagaatcgcaacgctaaaagagatcgtatttttgttagagtatataacgttatgaaacga
attaattgttttataaacaaaaatataaagaaatcgtccacagattccaattatcagttggcggtttttatgttaatggaaac
tatgttttttattagatttggtaaaatgaaatatcttaaggagaatgaaacagtagggttattaacactaaaaaataaaca
catagaaataagtcccgatgaaatagttatcaagtttgtaggaaaggacaaagtttcacatgaatttgttgttcataagtc
taatagactatataaaccgctattgaaactgacggatgattctagtcccgaagaatttctgttcaacaaactaagtgaac
gaaaggtatacgaatgtatcaaacagtttggtattagaatcaaggatctccgaacgtatggagtcaattatacgtttttat
ataatttttggacaaatgtaaagtccatatctcctcttccgtcaccaaaaaagttaatagcgttaactatcaaacaaactg
ctgaagtggtaggtcatactccatcaatttcaaaaagagcttatatggcaacgactattttagaaatggtaaaggataa
aaattttttagatgtagtatctaaaactacgttcgatgaattcctatctatagtcgtagatcacgttaaatcatctacggatg
gatgatatagatctttacacaaataattacaagaccgataaatggaaatggataagcgtatgaaatctctcgcaatga
cagctttcttcggagagctaaacacattagatattatggcattgataatgtctatatttaaacgccatccaaacaatacca
ttttttcagtggataaggatggtcagtttatgattgatttcgaatacgataattataaggcttctcaatatttggatctgaccct
cactccgatatctggagatgaatgcaagactcacgcatcgagtatagccgaacaattggcgtgtgtggatattattaaa
gaggatattagcgaatatatcaaaactactccccgtcttaaacgatttataaaaaaataccgcaatagatcagatactc
gtatcagtcgagatacagaaaagcttaaaatagctctagctaaaggcatagattacgaatatataaaagacgcttgtt
aataagtaaatgaaaaaaaactagtcgtttataataaaacacaatatggatgccaacatagtatcatcttctactattgc
aacgtatatagacgctttagcgaagaatgcttcagaattagaacagaggtctaccgcatacgaaataaataatgaatt
ggaactagtatttattaagccgccattaattactttgacaaatgtagtgaatatctctacgattcaggaatcgtttattcgatt
taccgttactaataaggaaggtgttaaaattagaactaagattccattatctaaggtacatggtctagatgtaaaaaatgt
acagttagtagatgctatagataacatagtttgggaaaagaaatcattagtgacggaaaatcgtcttcacaaagaatg
cttgttgagactatcgacagaggaacgtcatatatttttggattacaagaaatatggatcctctatccgactagaattagtc
aatcttattcaagcaaaaacaaaaaactttacgatagactttaagctaaaatattttctaggatccggtgcccagtctaa
aagttctttattacacgctattaatcatccaaagtcaaggcctaatacatctctggaaatagaattcacacctagagaca
atgaaaaagttccatatgatgaactaataaaggaattgacgactctatcacgtcatatatttatggcttctccagagaat
gtaattctttctccgcctattaacgcgcctataaaaacctttatgttgcctaaacaagatatagtaggtttggatctggaaa
atctatatgccgtaactaagactgacggaattcctataactatcagagttacatcaaaagggttgtattgttattttacacat
cttggttatattattagatatcctgttaagagaataatagattccgaagtagtagtctttggtgaggcagttaaggataaga
actggaccgtatatctcattaagctaatagagcctgtgaatgcaatcaatgatagactagaagaaagtaagtatgttga
atctaaactagtggatatttgtgatcggatagtattcaagtcaaagaaatacgaaggtccgtttactacaactagtgaag
tcgtcgatatgttatctacatatttaccaaagcaaccagaaggtgttattctgttctattcaaagggacctaaatctaacatt
gattttaaaattaaaaaggaaaatactatagaccaaactgcaaatgtagtatttaggtacatgtccagtgaaccaattat
ctttggagaatcgtctatctttgtagagtataagaaatttagcaacgataaaggctttcctaaagaatatggttctggtaag
attgtgttatataacggcgttaattatctaaataatatctattgtttggaatatattaatacacataatgaagtgggtattaagt
ccgtggttgtacctattaagtttatagcagaattcttagttaatggagaaatacttaaacctagaattgataaaaccatga
aatatattaactcagaagattattatggaaatcaacataatatcatagtcgaacatttaagagatcaaagcatcaaaat
aggagatatctttaacgaggataaactatcggatgtgggacatcaatacgccaataatgataaatttagattaaatcca
gaagttagttattttacgaataaacgaactagaggaccgttgggaattttatcaaactacgtcaagactcttcttatttctat
gtattgttccaaaacatttttagacgattccaacaaacgaaaggtattggcgattgattttggaaacggtgcggacctgg
aaaaatacttttatggagagattgcgttattggtagcgacggatccggatgctgatgctatagctagaggaaatgaaag
atacaacaaattaaactctggaattaaaaccaagtactacaaatttgactacattcaggaaactattcgatccgataca
tttgtctctagtgtcagagaagtattctattttggaaagtttaatatcatcgactggcagtttgctatccattattcttttcatccg
agacattatgctaccgtcatgaataacttatccgaactaactgcttctggaggcaaggtattaatcactaccatggacg
gagacaaattatcaaaattaacagataaaaagacttttataattcataagaatttacctagtagcgaaaactatatgtct
gtagaaaaaatagctgatgatagaatagtggtatataatccatcaacaatgtctactccaatgactgaatacattatca
aaaagaacgatatagtcagagtgtttaacgaatacggatttgttcttgtagataacgttgatttcgctacaattatagaac
gaagtaaaaagtttattaatggcgcatctacaatggaagatagaccgtctacaaaaaactttttcgaactaaatagag
gagccattaaatgtgaaggtttagatgtcgaagacttacttagttactatgttgtttatgtcttttctaagcggtaaataataat
atggtatgggttctgatatccccgttctaaatgcattaaataattccaatagagcgatttttgttcctataggaccttccaact
gtggatactctgtattgttaatagatatattaatacttttgtcgggtaacagaggttctacgtcttctaaaaataaaagtttgat
aacatctggcctgttcataaataaaaacttggcgattctatatatactcttattatcaaatctagccattgtcttatagatgtg
agctactgtaggtgtaccatttgattttctttctaatactatatatttctctcgaagaagttcttgcacatcatctgggaataaa
atactactgttgagtaaatcagttattttttttatatcgatattgatggacatttttatagttaaggataataagtatcccaaagt
cgataacgacgataacgaagtatttatacttttaggaaatcacaatgactttatcagatcaaaattaacaaaattaaag
gagcatgtatttttttctgaatatattgtgactccagatacatatggatctttatgcgtcgaattaaatgggtctagttttcagc
acggtggtagatatatagaggtggaggaatttatagatgctggaagacaagttagatggtgttctacatccaatcatat
atctgaagatatgcacactgataaatttgtcatttatgatatttatacgtttgattcgttcaagaataaacgattggtatttgta
caggtgcctccatcattaggagatgatagctatttgactaatccgttattgtctccgtattatcgtaattcagtagccagac
aaatggtcaatgatatgatttttaatcaagattcatttttaaaatatttattagaacatctgattagaagccactatagagtttc
taaacatataacaatagttagatacaaggataccgaagaattaaatctaacgagaatatgttataatagagataagttt
aaggcgtttgtattcgcttggtttaacggcgtttcggaaaatgaaaaggtactagatacgtataaaaaggtatctaatttg
atataatgaattcagtgactgtatcacacgcgccatatactattacttatcacgatgattgggaaccagtaatgagtcaat
tggtagagttttataacgaagtagccagttggctgctacgagacgagacgtcgcctattcctgataagttctttatacagtt
gaaacaaccgcttagaaataaacgagtatgtgtgtgtggtatagatccgtatccgaaagatggaactggtgtaccgtt
cgaatcaccaaattttacaaaaaaatcaattaaggagatagcttcatctatatctagattaaccggagtaattgattata
aaggttataaccttaatataatagacggggttataccctggaattattacttaagttgtaaattaggagaaacaaaaagt
cacgcgatctactgggataagatttccaagttactgctgcagcatataactaaacacgttagtgttctttattgtttgggtaa
aacagatttctcgaatatacgggcaaagttagaatccccggtaactaccatagtcggatatcatccagcggctagag
accgccaattcgagaaagatagatcatttgaaattatcaacgttttactggaattagacaacaaggcacctataaattg
ggctcaagggtttatttattaatgctttagtgaaattttaacttgtgttctaaatggatgcaactattagaggtaatgatgttat
ctttgttcttaagactataggtgtcccgtcagcgtgcagacaaaatgaagatccaagatttgtagaagcatttaaatgcg
acgagttagaaagatatattgagaataatccagaatgtacactattcgaaagtcttagggatgaggaagcatactctat
agtcagaattttcatggatgtagatttagacgcgtgtctagacgaaatagattatttaacggctattcaagattttattatcg
aggtgtcaaactgtgtagctagattcgcgtttacagaatgcggtgccattcatgaaaatgtaataaaatccatgagatct
aatttttcattgactaagtctacaaatagagataaaacaagttttcatattatctttttagacacgtataccactatggatac
attgatagctatgaaacgaacactattagaattaagtagatcatctgaaaatccactaaccagatcgatagacactgc
cgtatataggagaaaaacaactcttcgggttgtaggtactaggaaaaatccaaattgcgacactattcatgtaatgca
accaccgcatgataatatagaagattacctattcacttacgtggatatgaacaacaatagttattacttttctctacaacg
acgattggaggatttagttcctgataagttatgggaaccagggtttatttcattcgaagacgctataaaaagagtttcaaa
aatattcattaattctataataaactttaatgatctcgatgaaaataattttacaacggtaccactggtcatagattacgtaa
caccttgtgcattatgtaaaaaacgatcgcataaacatccgcatcaactatcgttggaaaatggtgctattagaatttac
aaaactggtaatccacatagttgtaaagttaaaattgttccgttggatggtaataaactgtttaatattgcacaaagaatttt
agacactaactctgttttattaaccgaacgaggagaccatatagtttggattaataattcatggaaatttaacagcgaag
aacccttgataacaaaactaattctgtcaataagacatcaactacctaaggaatattcaagcgaattactctgtccgag
gaaacgaaagactgtagaagctaacatacgagacatgttaatagattcagtggagaccgatacctatccggataaa
cttccgtttaaaaatggtgtattggacctggtagacggaatgttttactctggagatgatgctaaaaaatatacgtgtactg
tatcaaccggatttaaatttgacgatacaaagttcgtcgaagacagtccagaaatggaagagttaatgaatatcattaa
cgatatccaaccattaacggatgaaaataagaaaaatagagagttgtacgaaaaaactttatctagttgtttatgcggt
gctaccaaaggatgtttaacattcttttttggagaaactgcaactggaaagtcgacaaccaaacgtttgttaaagtctgct
atcggtgacctgtttgttgagacgggtcaaacaattttaacagatgtattggataaaggacctaatccatttatcgctaac
atgcatttgaaaagatctgtattctgtagcgaactacctgattttgcctgtagtggatcaaagaaaattagatctgacaat
attaaaaagttgacagaaccttgtgtcattggaagaccgtgtttctccaataaaattaataatagaaaccatgcgacaa
tcattatcgatactaattacaaacctgtctttgataggatagataacgcattaatgagaagaattgccgtcgtgcgattca
gaacacacttttctcaaccttctggtagagaggctgctgaaaataatgacgcgtacgataaagtcaaactattagacg
aggggttagatggtaaaatacaaaataatagatatagattcgcatttctatacttgttggtgaaatggtacaaaaaatat
catgttcctattatgaaactatatcctacaccggaagagattccggactttgcattctatctcaaaataggtactctgttagt
atctagctctgtaaagcatattccattaatgacggacctctccaaaaagggatatatattgtacgataatgtggttactctt
ccgttgactactttccaacagaaaatatccaagtattttaattctagactatttggacacgatatagagagcttcatcaata
gacataagaaatttgccaatgttagtgatgaatatctgcaatatatattcatagaggatatttcatctccgtaaatatatgct
catatatttatagaagatatcacatatctaaatgaataccggaatcatagatttatttgataatcatgttgatagtataccaa
ctatattacctcatcagttagctactctagattatctagttagaactatcatagatgagaacagaagcgtgttattgttccat
attatgggatcaggtaaaacaataatcgctttgttgttcgccttggtagcttccagatttaaaaaggtttacattctagtgcct
aatattaacattttgaaaatttttaattataatatgggtgtagctatgaacttgtttaatgacgaattcatagctgagaatatct
ttattcattccacaacaagtttttattctcttaattataacgataacgtcattaattataacggattatctcgctacaataactct
atttttatcgttgatgaggcacataatatctttgggaataatactggagaacttatgaccgtgataaaaaataaaaacaa
gattccttttttactattgtctggatctcccattactaacacacctaatactctgggtcatattatagatttaatgtccgaagag
acgatagattttggtgaaattattagtcgtggtaagaaagtaattcagacacttcttaacgaacgaggtgtgaatgtactt
aaggatttgcttaaaggaagaatatcatattacgaaatgcctgataaagatctaccaacgataagatatcacggacgt
aagtttctagatactagagtagtatattgtcacatgtctaaacttcaagagagagattatatgattactagacgacagcta
tgttatcatgaaatgtttgataaaaatatgtataacgtgtcaatggcagtattgggacaacttaatctgatgaataatttag
atactttatttcaggaacaggataaggaattgtacccaaatctgaaaataaataatggcgtgttatacggagaagaatt
ggtaacgttaaacattagttccaaatttaaatactttattaatcggatacagacactcaacggaaaacattttatatactttt
ctaattctacatatggcggattggtaattaaatatatcatgctcagtaatggatattctgaatataatggttctcagggaact
aatccacatatgataaacggcaaaccaaaaacatttgctatcgttactagtaaaatgaaatcgtctttagaggatctatt
agatgtgtataattctcctgaaaacgatgatggcagtcaattgatgtttttgttttcgtcaaacattatgtccgaatcctatact
ctgaaagaggtaaggcatatttggtttatgactatcccagatactttttctcaatacaaccaaattcttggacgatctattag
aaaattctcttacgccgatatttctgaaccagttaatgtatatcttttagccgccgtatattccgatttcaatgacgaagtga
cgtcattaaacgattacacacaggatgaattaattaatgttttaccatttgacatcaaaaagctgttgtatctaaaatttaa
gactaaagaaacgaatagaatatactctattcttcaagagatgtctgaaacgtattctcttccaccacatccatcaattgt
aaaagttttattgggagaattggtcagacaatttttttataataattctcgtattaagtataacgataccaagttacttaaaat
ggttacatcagttataaaaaataaagaagacgctaggaattacatagatgatattgtaaacggtcacttctttgtatcga
ataaagtatttgataaatctcttttatacaaatacgaaaacgatattattacagtaccgtttagactttcctacgaaccatttg
tttggggagttaactttcgtaaagaatataacgtggtatcttctccataaaactgatgagatatataaagaaataaatgtc
gagctttgttaccaatggataccttccagttacattggagccacacgagctgacgttagacataaaaactaatattagg
aatgccgtatataagacgtatctccatagagaaattagtggtaaaatggccaagaaaatagaaattcgtgaagacgt
ggaattacctctcggcgaaatagttaataattctgtagttataaacgttccgtgtgtaataacctacgcgtattatcacgttg
gggatatagtcagaggaacattaaacatcgaagatgaatcaaatgtaactattcaatgtggagatttaatctgtaaact
aagtagagattcgggtactgtatcatttagcgattcaaagtactgcttttttcgaaatggtaatgcgtatgacaatggcag
cgaagtcactgccgttctaatggaggctcaacaaggtatcgaatctagttttgtttttctcgcgaatatcgttgactcataa
aaaagagaatagcggtaagtataaacacgaatactatggcaataattgcgaatgttttattcccttcgatatatttttgata
atatgaaaaacatgtctctctcaaatcggacaaccatctcataaaatagttctcgcgcgctggagaggtagttgctgctc
gtataatctccccagaataatatacttgcgtgtcgtcgttcaatttatacggatttctatagttctctgttatataatgcggttttc
catcatgattagacgacgacaatagtgttctaaatttagatagttgatcagaatgaatgtttattggcgttggaaaaattat
ccatacagcgtctgcagagtggttgatagttgttcctagatatgtaaaataatccaacttactaggcagcaaattgtctag
ataaaatactgaatcaaacggtgcagacgtattggtggatctaatggaatccaattgattaactatcttttgaaaatatac
atttttatgatccgatacttgtaagaatatagaaataatgataagtccatcatcgtgtttttttgcctcttcataagaactatatt
ttttcttattccaatgaacaagattaatctctccagagtatttgtacacatctatcaagtgattggatccataatcgtcttccttt
ccccaatatatacgtagtgatgataacacatattcattggggagaaaccctccacttatatatcctcctttaaaattaatcc
ttactagttttccagtgttctggatagtggttggtttcgactcattataatgtatgtctaacggcttcaatcgcgcgttagaaatt
gcttttttagtttctatattaataggagatagttgttgcggcatagtaaaaatgaaatgataactgtttaaaaatagctcttag
tatgggaattacaatggatgaggaagtgatatttgaaactcctagagaattaatatctattaaacgaataaaagatattc
caagatcaaaagacacgcatgtgtttgctgcgtgtataacaagtgacggatatccgttaataggagctagaagaactt
cattcgcattccaagcgatattatctcaacaaaattcagattctatctttagagtatccactaaactattacggtttatgtact
acaatgaactaagagaaatctttagacggttgagaaaaggttctatcaacgatatcgatcctcactttgaagagttaat
attattgggtggtaaactagataaaaaggaatctattaaagattgtttaagaagagaattaaaagaggaaagtgatga
acgtataacagtaaaagaatttggaaatgtaattctaaaacttacaacacgggataaattatttaataaagtatatataa
gttattgcatggcgtgttttattaatcaatcgttggaggatttatcgcatactagtatttacaatgtagaaattagaaagatta
aatcattaaatgattgtattaacgacgataaatacgaatatctgtcttatatttataatatgctagttaatagtaaatgaactt
ttacagatctagtataattagtcagattattaagtataatagacgactagctaagtctattatttgcgaggatgactctcaa
attattacactcacggcattcgttaaccaatgcctatggtgtcataaacgagtatccgtgtccgctattttattaactactgat
aacaaaatattagtatgtaacagacgagatagttttctctattctgaaataattagaactagaaacatgtctagaaagaa
acgattatttctgaattattccaattatttgtccaaacaggaaagaagtatactatcgtcatttttttctctagatccagctact
actgataatgatagaatagatgctatttatccgggtggcatacccaaaaggggtgagaatgttccagagtgtttatcca
gggaaattaaagaagaagttaatatagacaattcttttgtattcatagacactcggttttttattcatggcatcatagaagat
accattattaataaattttttgaggtaatcttctttgtcggaagaatatctttaacgagtgatcaaatcattgatacatttaaaa
gtaatcatgaaatcaaggatctaatatttttagatccgaattcaggtaatggactccaatacgaaattgcaaaatatgct
ctagatactgcaaaactcaaatgttatggccatagaggatgttattacgaatcattaaaaaaattaactgaggatgattg
attagaaaatataaattaatttaccatcgtgtatttttataacgggattgtccggcatatcatgtagatagttaccgtctacat
cgtatactcgaccatctacgcctttaaatcctctatttattgacattaatctattagaattggaataccaaatattagtaccct
caattagtttattggtaatattttttttagacgatagatcgatggctcttgaaaccaaggttttccaaccggactcattgtcgat
cggtgagaagtctttttcattagcatgaatccattctaatgatgtatgtttaaacactctaaacaattggacaaattcttttga
tttgctttgaatgatttcaaataggtcttcgtctacagtaggcataccattagataatctagccattataaagtgcacgtttac
atatctacgttctggaggagtaagaacgtgactattgagacgaatggctcttcctactatctgacgaagagacgcctcg
ttccatgtcatatctaaaatgaagatatcattaattgagaaaaaactaataccctcgcctccactagaagagaatacgc
atgttttaatgcattctccgttagtgtttgattcttggttaaactcagccaccgccttgattctagtatcttttgttctagatgaga
actctatattagagataccaaagactttgaaatatagtaataagatttctattcctgactgattaacaaatggttcaaaga
ctagacatttaccatgggatgctaatattcccaaacatacatctataaatttgacgcttttctcttttaattcagtaaatagag
agatatcagccgcactagcatcccctttcaatagttctccctttttaaaggtatctaatgcggatttagaaaactctctatct
cttaatgaatttttaaaatcattatatagtgttgctatctcttgcgcgtattcgcccggatcacgattttgtctttcaggaaagct
atcgaacgtaaacgtagtagccatacgtctcagaattctaaatgatgatatacctgtttttatttcagcgagtttagccttttg
ataaatttcttcttgctttttcgacatattaacgtatcgcattaatactgttttcttagcgaatgatgcagacccttctacgtcatc
aaaaatagaaaactcgttattaactatgtacgaacataggcctcctagtttggagactaattctttctcatcaactagacg
tttattctcaaatagcgattggtgttgtaaggatcctggtcgtagtaagttaaccaacatggtgaattcttgcacactattaa
cgataggtgtagccgataaacaaatcatcttatggttttttaatgcgatggtcttagataaaaaattatatactgaacgagt
aggacggatcttaccatcttctttgattaatgatttagaaatgaagttatgacattcatcaataatgacgcatattctactctt
ggaattaatagttttgatattagtaaaaaatttatttctaaaattttgatcatcgtaattaataaaaatacaatccttcgttatct
ctggagcgtatctgagtatagtgttcatccaaggatcttctatcaaagcctttttcaccaataagataatagcccaattcgt
ataaatatccttaagatgtttgagaatatatacagtagtcattgttttaccaacacccgtttcatggaacaataaaagaga
atgcatactgtctaatcctaagaaaactcttgctacaaaatgttgataatccttgaggcgtactacgtctgttcccatcattt
caacaggcatattagtagttctgcgcaatgcataatcgatataggccgcgtgtgatttactcatttatgagtgataagtaa
taactatgttttaaaaatcacagcagtagtttaactagtcttctctgatgtttgttttcgatactttttgaatcagaagtcatact
agaataaagcaacgagtgaacgtaatagagagcttcgtatactctattcgaaaactctaagaacttattaatgaattcc
gtatccactggattgtttaaaatactaaattgaacactgttcacatccttccaagaagaagacttagtgacggacttaac
atgagacataaataaatccaaattttttttacaaacatcactagccaccataatggcgctatctttcaaccagctatcgctt
acgcattttagcagtctaacatttttaaagagactacaatatattctcatagtatcgattacacctctaccgaataaagttg
gaagtttaataatacaatatttttcgtttacaaaatcaaataatggtcgaaacacgtcgaaggttaacatcttataatcgct
aatgtatagattgttttcagtgagatgattattagatttaatagcatctcgttcacgtttgaacagtttattgtgtgcgctgaggt
cggcaactacggcgtccgctttagtactcctcccataatactttacgctattaatctttaaaatttcatagactttatctagat
cgctttctggtaacatgatatcatgtgtaaaaagttttaacatgtcggtcggcattctatttagatcattaactctagaaatct
gaagaaagtaattagctccgtattccagactaggtaatgggcttttacctaaagacaagttaagttctggcaatgtttcat
aaaatggaagaaggacatgcgttccctcccggatattttttacaatttcatccatttacaactctatagtttgttttcattattat
tagttattatctcccataatcttggtaatacttaccccttgatcgtaagataccttatacaggtcattacatacaactaccaat
tgtttttgtacataatagattggatggttgacatccatggtggaataaactactcgaacagatagtttatctttccccctagat
acattggccgtaatagttgtcggcctaaagaatatctttggtgtaaagttaaaagttagggttcttgttccattattgctttttgt
cagtagttcattataaattctcgagatgggtccgttctctgaatatagaacatcatttccaaatctaacttctagtctagaaa
taatatcggtcttattcttaaaatctattcccttgatgaagggatcgttaatgaacaaatccttggcctttgattcggctgatct
attatctccgttatagacgttacgttgactagtccaaagacttacaggaatagatgtatcgatgatgttgatactatgtgat
atgtgagcaaagattgttctcttagtggcatcactatatgttccagtaatggcggaaaactttttagaaatgttatatataaa
agaattttttcgtgttccaaacattagcagattagtatgaagataaacactcatattatcaggaacattatcaatttttacat
acacatcagcatcttgaatagaaacgataccatcttctggaacctcaacaatctcggcagactccggataaccagtc
ggtgggccatcactaacaataactagatcatccaacaatctactcacatatgcatctatataatctttttcatcttgtgagta
ccctggatacgaaataaatttattatccgtatttccataataaggtttagtataaacagagagcgatgttgccgcatgaac
ttcagttacagtcgccgttggttggtttatttgacctattactctcctaggtttctctataaacgatggtttaatttgtacattctta
accatatatccaataaagctcaattcaggaacataaacaaattctttgttgaacgtttcaaagtcgaacgaagagtcac
gaataacgatatcggatactggattgaaggttaccgttacggtaatttttgaatcggatagtttaagactgctgaatgtatc
ttccacatcaaacggagttttaatataaacgtatactgtagatggttctttaatagtgtcattaggagttaggccaatagaa
atatcattaagttcactagaatatccagagtgtttcaaagcaattgtattattgatacaattattatataattcttcgccctcaa
tttcccaaataacaccgttacacgaagagatagatacgtgattaatacatttatatccaacatatggtacgtaaccgaat
cttcccatacctttaacttctggaagttccaaactcagaaccaaatgattaagcgcagtaatatactgatccctaatttcg
aagctagcgatagcctgattgtctggaccatcgtttgtcataactccggatagagaaatatattgcggcatatacaaagt
tggaatttgactatcgactgcgaagacattagaccgtttaatagagtcatccccaccgatcaaagaattaatgatagtat
tattcattttctatttaaaatggaaaaagcttacaataaactccgtagagaaatatctataatttgtgagttttccttaaagta
acagcttccgtaaacgccgtctttatctcttagtaggtttattgtatttatgaccttttccttatcttcatagaatactaaaggca
acaaagaaatttttggttcttctctaagagctacgtgagacttaaccatagaagccaacgaatccctacatattttagaa
cagaaatacccaacttcaccacccttgaatgtctcaatactaataggtctaaaaaccaaatcttgattacaaaaccaa
cacttatcaattacactatttgtcttaatagacacatctgccatagatttataatactttggtagtatacaagcgagtgcttctt
ctttagcgggcttaaagactgctttaggtgctgaaataaccacatctggaaggcttactcgcttagccatttaattacgga
actatttttttatacttctaatgagcaagtagaaaacctctcatctacaaaaacatactcgtgtccataatcctctaccatag
ttacacgttttttagatctcatatgtgctaaaaagttttcccatactaattggttactattatttttcgtataatttttaacagtttga
ggttttagatttttagttacagaagtgatatcgaatattttatccaaaaagaatgaataattaattgtcttagaaggagtgtttt
cttggcaaaagaataccaagtgcttaaatatttctactacttcattaatcttttctgtactcagattcagtttctcatcttttactt
gattgattatttcaaagactaacttataatcctttttatttattctctcgttagccttaagaaaactagatacaaaatttgcatct
acatcatccgtggatatttgatttttttccatgatatccaagagttccgagataatttctccagaacattgatgagacaataa
tctccgcaatacatttctcaaatgaataagtttattagacacatggaagtttgactttttttgtacctttgtacatttttgaaatac
agactcgcaaaaaatacaatattcatatccttgttcagatactataccgttgtgtctacaaccgctacataatcgtagattc
atgttaacactctacgtatctcgtcgtccaatattttatataaaaacattttatttctagacgttgccagaaaatcctgtaatat
ttttagttttttgggctgtgaataaagtatcgccctaatatggttaccgtcctccgccaatatagtagttaaattatccgcaca
tgcagaagaacaccgcttaggcggattcagtacaatgttatatttttcgtaccaactcatttaaatatcataatctaaaata
gttctgtaatatgtctagcgctaatatattgatcataatcctgtgcataaattaagatacaacaatgtctcgaaatcatcga
catggcttcttccatagttagaagatcgtcgtcaaagttagcaacgtgattcatcaacatttgctgttttgaggcagcaaat
actgaaccgtcgccattcaaccattcataaaaaccatcgtctgaatccattgataatttcttgtactggtttttgagagctcg
catcaatctagcatttctagctcccggattgaaaacagaaagaggatcgtacatccagggtccattttctgtaaataga
atcgtataatgtcccttcaagaagatatcagacgatccacaatcaaagaattggtctccgagtttgtaacaaactgcgg
actttaacctatacatgataccgtttagcatgatttctggtgatacgtcaatcggagtatcatctattagagatctaaagcc
ggtgtaacattctccaccaaacatattcttattctgacgtcgttctacataaaacatcattgctccattaacgataacaggg
gaatgaacagcactacccatcacattagttcccaatggatcaatgtgtgtaactccagaacatcttccatatcctatgtta
ggaggagcgaacaccactcttccactattgccatcgaatgccatagaataaatatccttggaattgatagaaatcgga
ctgtcggatgttgtgatcatcttcataggattaacaactatgtatggtgccgcctgaagtttcatatcgtaactgatgccgttt
ataggtctagccacagaaaccaacgtaggtctaaatccaactatagacaaaatagaagccaatatctgttcttcatct
gtcataacttgagagcatccagtatgaataatcttcattagatggggatctaccgcatcatcatcgttacaataaaaaatt
cccattctaatgttcataattgcttttctaatcatggtatgcatgtttgctctctgaatctctgtggaaattagatctgatacacc
tgtaatcactatcggattatcctccgtaagacgattaaccaacaacatataattataagactttacttttctaaattcataaa
gttgctggattaggctataggtgtctccatgtacatacgcgttctcgagcgcaggaagtttaataccgaatagtgccatc
agaataggatgaatatagtaattagtttctggttttctataaataaaagacaaatcttgtgaactagacatatcggtaaaa
tgcatggattggaatcgtgtagtcgacagaagaatatgatgattagatggagagtatattttatctaactctttgagttggt
caccgattctaggactagctcgagaatgaataagtactaaaggatgagtacatttcacagaaacactagcattgttca
atgtgctctttacatgggtaaggagttgaaatagctcgtttctatttgttctgacaatatttagtttattcataatgttaagcatat
cctgaatagtaaagttagatgtgtcatacttgttagtagttagatatttagcaattgcattcccatcatttctcaatctcgtact
ccaatcatgcgtggatgctacttcgtcgatggaaaccatacaatcctttttgataggctgttgagattgattatttcctgcac
gtttaggtttggtacgttgatttctagcccctgcggatataaagtcatcgtctacaattttggataatgaattgcatacactac
aagacaaagatttatcagaagtgtgaatatgatcttcatctaccaaagaaagagtttgattagtataactagattttagtc
ctgcgttagatgttaaaaaaacatcgctattgaccacggcttccattatttatattcgtagtttttactcgaaagcgtgatttta
atattcaatcttattacttttggaatcgttcaaaacctttgactaattgtagaatttgatctattgccctacgcgtatactccctt
gcatcatatacgttcgtcaccagatcgtttgtttcggcctgaagttggtgcatatctctttcaacattcgacatgagatcctta
agggccatatcgtctagattttgttgagatgctgctcctggatttggattttgttgtgctgttgtacatactgtaccaccagtag
gtgtaggagtacatacagtggccacaataggaggttgaggaggtgtaaccgttggagtagtacaagaaatacttcca
tccgattgttgtgtacatgtagttgttggtaacgtctgagaaggttgggtagatggcggcgtcgtcgtcttttgatctttattaa
atttagagataatatcctgaacagcattgctcggcgtcaacgctggaaggagtgaactcgccggcgcatcagtatctg
cagacagccaatcaaaaagattagacatatcagatgatgtattagtttgttgtcgtggttttggtgtaggagccggtgtag
ctgttggaaccggctgtggagttatatgaatagttggttgtagcggttggataggctgtctgctggcggccatcatattatc
tctagctagttgttctcgcaactgtctttgataatacgactcttgagactttagtcctatttcaatcgcttcatcctttttcgtatcc
ggatccttttcttcagaataatagattgacgactttggtgtagaggattctgccagcccctgtgagaacttgttaaagaagt
ccatttaaggctttaaaattgaattgcgattataagattaaatggcagacacagacgatattatcgactatgaatccgat
gatctcaccgaatacgaggatgatgaagaagaggaagaagatggagagtcactagaaactagtgatatagatccc
aaatcttcttataagattgtagaatcagcatccactcatatagaagatgcgcattccaatcttaaacatatagggaatcat
atatctgctcttaaacgacgctatactagacgtataagtctatttgaaatagcgggtataatagcagaaagctataactt
gcttcaacgaggaagattacctctagtttcagaattttctgacgaaacgatgaagcaaaatatgctacatgtaattatac
aagagatagaggagggttcttgtcctatagtcatcgaaaagaacggagaattgttgtcggtaaacgattttgacaaag
atggtctaaaattccatctagactatattatcaaaatttggaaacttcaaaaacgatattagaatttatacgaatatcgttct
ctaaatgtcacaatcaagtctcgcatgttcagcaatttattgtcgtactttatatcgtgttcattaacgatatcttgcaaaata
gtaatgattctatcttccttcgatagatattcttcagagattattgtcttatattctttcttgttatcagatatgaatttgataagact
ttgaacattattgatacccgtctgtttaattttttctacagatattttagttttggcagattctatcgtatctgtcaatagacatcca
acatcgacattcgacgtcaattgtctataaatcaacgtataaattttagaaataacattagcgaattgttgtgcattgatgt
cgttattctgaaacagtatgattttaggtagcattttcttaacaaagagaacgtatttattgttactcagttgaacagatgata
tatccagattactaacgcatctgattccatataccaaactttcagaagaaatggtgtacaattgtttgtattcattcaatgtct
ctttttcagaaattagtttagagtcgaatactgcaataattttcaagagatagttttcatcagataagattttatttagtgtaga
tatgataaaactattgttttgttggagaacttgatacgccgcgttctctgtagtcgacgctctcaaatgggaaacaatctcc
attatttttttggaatcggatacaatatcttcggtatcttgacgcaatctagtatacatagagttaagagaaattagagtttgt
acattaagcaacatgtctctaaatgtggctgcaaacttttccttttccacatcatctagtttattatataccgatttcacaacg
gcaccagatttaaggaaccagaatgaaaaactctgataactacaatatttcatcatagttacgattttatcatcttctatag
ttggtgtaatagcgcatacctttttctccaagactggaaccaacgtcataaaaatgtttaaatcaaaatccatatcaacat
ctgatgcgctaagaccagtctcgcgttcaagattatctttactaatggtgacgaactcatcgtatagaactctaagtttgtc
cattatttatttacagatttagttgtttaatttatttgtgctcttccagagttgggatagtatttttctaacgtcggtattatattattag
gatctacgttcatatgtatcataatattaatcatccacgttttgataaatctatctttagcttctgaaataacgtatttaaacaa
aggagaaaaatatttagctacggcatcagacgcaataacattttttgtaaatgtaacgtatttagacgacagatcttcgtt
aaaaagttttccatctatgtagaatccatcggttgttaacaccattcccgcgtcagattgaataggagtttgaatagtttgttt
tggaaatagatccttcaataacttatagttgggtgggaaaaaatcgattttatcactagactctttcttttttactatcattacct
catgaactatttcttgaatgagtatatgtattttctttcctatatcggacgcgttcattggaaaatataccatgtcgttaactata
agaatatttttatcctcgtttacaaactgaataatatcagatgtagttcgtaaacgaactatatcatcaccagcacaacat
ctaactatatgatatccactagtttcctttagtcgtttattatcttgttccatattagcagtcattccatcatttaagaaggcgtc
aaagataatagggagaaatgacattttggattctgttacaactttaccaaaattaaggatatacggacttactatctttttct
caacgtcgatttgatgaacacacgatgaaaatgtacttcgatgagattgatcatgtagaaaacaacaagggatacaa
tatttccacatatcatgaaatatattaagaaatcccaccttattatatttccccaaaggatccatgcatgtaaacattatgcc
gttatcattaataaagacttctttctcatcggatctgtaaaagttgttactgatttttttcattccaggatctagataattaataat
gatgggttttctattcttattctttgtattttggcatatcctagaccagtaaacagtttccactttggtaaaatcagcagacttttg
aacgctattaaacatggcattaatggcaataactaaaaatgtaaaatatttttctatgttaggaatatggtttttcactttaat
agatatatggtttttggccaaaatgatagatatttttttatccgaggatagtaaaatattattagtcgccgtctctataaaaat
gaagctagtctcgatatccaattttattctagaattgataggagtcgccaaatgtaccttatacgttatatctcccttgatgc
gttccatttgtgtatctatatcggacacaagatctgtaaatagttttacgttattaatcatcacggtatcgccgtcgctagata
acgctaatgtaccatccaagtcccaaatggagagatttaactgttcatcgtttagaataaaatgattaccggtcatattaa
taaagtgttcatcgtatctagataacaacgacttataattaatgtccaagtcttgaactcgctgaatgatcttttttaaccca
gttagttttagattggtacgaaatatattgttaaactttgattctacagtaatgtccaaatctagttgtggaaatacttccatca
acattgtttcaaacttgataatattattatctacatcttcatacgatccaaattccggaatagatgtatcgcacgctctggcc
acccagataaccaaaaagtcacacgctccaggatatacattgtataaaaagctatcgttttttagtagtgtttttttctgagt
atatacgaagggattaaaaatagtattatcaacgtaactatattccaaattattcttatgagaatagataataatatcgtcc
ttaatatctaacaaatttcctaaatatccctttaattgagtcattcgaagcgtcaatagaatatgtctcttaactatttccggct
gttgtatatttaaatgacttcgtaaaaaataatatatgggcgacttctcatctatgtaatcatatggagtgagatatagggct
cgttctacctcctgccccttacccacctgtaataccaattgcggacttactatatatcgcatatttatatcgtggggtaaagt
gaaaatctactaccgatgatgtaagtcttacaatgttcgaaccagtaccagatcttaatttggaggcctccgtagaacta
ggggaggtaaatatagatcaaacaacacctatgataaaggaaaatagcggttttatatcccgtagtagacgtctattc
gcccatagatctaaggatgatgagagaaaactagcactacgattctttttacaaagactttattttttagatcatagagag
attcattatttgttcagatgcgttgacgctgtaaaagacgtcactattaccaaaaaaaataacattatcgtggcgccttata
tagcacttttaactatcgcatcaaaaggatgcaaacttacagaaacaatgattgaagcattctttccagaactatataat
gaacatagtaagaaatttaaattcaactctcaagtatccatcatccaagaaaaactcggataccagtttggaaactatc
acgtttatgattttgaaccgtattactctacagtagctctggctattcgagatgaacattcatctggcatttttaatatccgtca
agagagttatctggtaagttcattatctgaaataacatatagattttatctaattaatctaaaatctgatcttgttcaatggagt
gctagtacgggcgctgtaattaatcaaatggtaaatactgtattgattacagtgtatgaaaagttacaactggtcataga
aaatgattcacaatttacatgttcattggctgtggaatcaaaacttccaataaaattacttaaagatagaaatgaattattt
acaaaattcatcaacgagttaaaaaagaccagttcattcaagataagcaaacgcgataaggatacgctactaaaat
attttacttaggactggagttagaatttatagacgactcatttcgtttatcattattagtattcttcttgttatcttgttcagaaatat
acagcaatgctatgcctaatactaaatacattatcatgcttgcaatggctctaacaacgacgaaccaaaatgaatttgg
tcgtagcttttgttcacaaaaatacataaagaaatgtctacataaatctatggcgccattggctacttgaaatagcgcca
gtcctcctacagattttaatatagctgtataacatgacatttattcatcatcaaaagagacagagtcaccatctgtcatattt
agattttttttcatgtgttcaaagtatcctctactcatttcattataatagtttatcatacttagaattttaggacggatcaatgag
taagacttgactagatcgtcagtagtaatttgtgcatcgtctattctgcatccgcttcgtcgaataatgtatagcatcgctttg
agattctccatagctatcaagtctttatacaatgacatggaaatatctgtgaatactttatacttctccaacatcgatgcctt
aacatcatcgcctactttagcattgaaaatacgttctattgtgtagatggatgtagcaagatttttaaacaacaatgccattt
tacacgatgattgcctcaagtctccaatcgtttgtttagaacgattagctacagagtccaatgcttggctgactagcatatt
attatctttagaaattgtattcttcaatgaggcgtttatcatatctgtgatttcgttagtcatattacagtctgactgggttgtaat
gttatccaacatatcacctatggatacggtacacgtaccagcatttgtaataatcctatctaagatgttgtatggcattgcg
cagaaaatatcttctcctgtaatatttccactctcgataaatctactcagattattcttaaatgccttattctctggagaaaag
atatcagtgtccatcatttcattaatagtatacgcagaaaagataccacgagtatcaattctatccaagatacttatcggtt
ccgagtcacagataatggtttcctctccttcgggagatcctgcatagaaatatctaggacaatagtttctatactgtctgta
actctgataatctctaaagtcactaactgataccatgaaattgagaagatcaaacgctgaagtaattaatttttctgcctc
gtttttactacaactagttttcatcaatgtagtgacgatgtattgtttagttactcttggtctaatactgatgatagagatattatt
acttcccataatggatcttctagtagtcaccttaaagcccattgatgcaaatagcagatagataaagtcttggtatgactc
ctttctaatatagtacggactacctttgtcacccaactttatacccacataagccataacaacctctttaatagccgtttcat
gaggtttatcagccatgagcctgagtagttggaagaatctcatgaatcctgtctcagaaagtcctatatgcatgatagatt
tatctttcctgggaaactctcgtatagtcatagatgaaatactcttcaaagtttctgaaataagattagtaacagtcttacct
ccgactactctaggtaacaaacaaactctaataggtgttttctctgcggagataatatcagaaaggatagagcaataa
gtagtattattgtgattataaagaccgaatacataacaggtagaatttataaacatcatgtcctgaaggtttttagacttgta
ttcctcgtaatccataccgtcccaaaacatggatttggtaactttgatagccgtagatctttgttccttcgccaacaggttaa
agaaattaataaagaatttgtggtttctacctatgtctacaaattgcacgtttggaagcgccacggttacattcactgcag
cattttgaggatcgcgagtatgaagtacgatgttattgtttactggtatatctggaaagaattctaccagtctaggaataag
agattgatatcgcatagaaatacaaaagttcataatctcatcatctaagagcattttgttaccattgtaataaatatccact
ctgtcatatgtataaatgaagtactgttcaaacatgatgagatgtttatatgttggcatagtagtgagatctacgtttggtaa
tggcaatgtattaagattaactccataatgtctagcagcatctgcgatgttataagcgttgtcaaagcggggtcgatcttgt
gctgttatatattgtctaacacctataagattatcaaaatcttgtctgcttaatacaccgttaacaatttttgccttgaattctttt
attggtgcattaataacatccttatagaggatgttaaacaaataagtgttatcaaagttaagatctggatatttcttttctgct
agaacatccattgagtcggagccatctggtttaatataaccaccgataaatctagctctgtattctgtatccgtcaatctaa
tattaagaaggtgttgagtgaaaggtggaagatcgtaaaagctgtgagtattaatgataggattagtttccgaactaatg
ttaattggggtattaataatatctatatttccagcgttaagtgtaacattaaacagttttaattcacgtgacgtggtatcaatta
aataattaatgcccaatttggatatagcagcctgaagctcatcttgtttagttacggatcctaatgagttattaagcaatat
atcgaacggatgaacgaaggttgttttaagttggtcacatactttgtaatctagacatagatgcggaagaacggtagaa
actatacgaaataaatattcagagtcctctaattgatcaagagtaactattgacttaataggcatcatttatttagtattaaa
tgacgaccgtaccagtgacggatatacaaaacgatttaattacagagttttcagaagataattatccatctaacaaaaa
ttatgaaataactcttcgtcaaatgtctattctaactcacgttaacaacgtggtagatagagaacataatgccgccgtagt
gtcatctccagaggaaatatcctcacaacttaatgaagatctatttccagatgatgattctccggccactattatcgaacg
agtacaacctcatactactattattgacgatactccacctcctacgtttcgtagagagttattgatatcggaacaacgtca
acaacgagaaaaaagatttaatattacagtatcgaaaaatgctgaagcaataatggaatctagatctatgatatcttct
atgccaacacaaacaccatccttgggagtagtttatgataaagataaaagaattcagatgttggaggatgaagtggtt
aatcttagaaatcaacgatctaatacaaaatcatctgataatttagataattttaccagaatactatttggtaagactccgt
ataaatcaacagaagttaataagcgtatagccatcgttaattatgcaaatttgaacgggtctcccttatcagtcgaggac
ttggatgtttgttcagaggatgaaatagatagaatctataaaacgattaaacaatatcacgaaagtagaaaacgaaa
aattatcgtcactaacgtgattattattgtcataaatattatcgagcaagcattgctaaaactcggatttgaagaaatcaa
aggactgagtaccgatatcacttcagaaattatcgatgtggagatcggagatgactgcgatgctgtagcatcaaaact
aggaatcggtaacagtccggttcttaatattgtattgtttatactcaagatattcgttaaacgaattaaaattatttaatttaat
acattcccatatccagacaacaatcgtctggattaatctgttcctgtcgtctcataccggacgacatattaatctttttattag
taggcatctttttagatggtttctttttcccagcattaactgagtcgatacctagaagatcgtgattgatctctccgaccattcc
acgaacttctaattggccgtctctgacggtaccataaactattttaccagcattagtaacagcttggacaatctgaccatc
catcgcattgtacgatgtagtagtaactgttgttctacgtctaggagcaccagaagtatttttggagcccttggatgttgatg
tagaagaagacgaggattttgattttggtttacatgtaatacattttgtatcacatgcgccggcagtcacatctgtttgaga
attaagattattgttgcctcctttgacggctgcatctccaccgatttgcgctagtagatttttaagctgtggtgtaatcttattaa
ctgtttcgatataatcatcgtaactgcttctaacggctaaattttttttatccgccatttagaagctaaaaatatttttatttatgc
agaagatttaactagattatacaatgaactaatatgatccttttccagattatttacaaacttggtattttttggttctggagga
ggcgaatttaaattcggacttggatttggattttgtgggttcttgatcttattatacagcgcatataggatggcgacggtaac
tgctacgcaaataccgatcaacaaaagaataccaatcatttattgacaataacttcactattgatcaagtatgcaatata
tcatcttttcactaaataagtagtaataatgattcaacaatgtcgagatatatggacgataataatttagttcatggaaatat
cgctatgattggtatgaatgactccgctaactctgtggggcgcgcagtgctttccccacatagaataaattagcattccg
actgtgataataataccaagtataaacgccataatactcaatactttccatgtacgagtgggactggtagacttactaaa
gtcaataaaggcgaagatacacgaaagaatcaaaagaatgattccagcgattagcacgccggaaaaataatttcc
aatcataagcatcatatccatttaactaataaaaattttaaatcgccgaatgaacaaagtggaatataaaccatataaa
aacaatagtttgtactgcaaaaataatatctatttttgttttcgaagatatggtaaaattaaatagtagtacacagcatgtta
taactaacagcagcaacggctcgtaattacttatcatttactagacgaaaaggtggtgggatattttcttgctcaaataat
acgaatatatcacccatccattttatgcgatgtttatatactctaatctttaatagatctatagacgacgggtttaccaacaa
tatagattttatcgattcatctaatttaaacccttccttaaacgtgaatgatctattatctggcataacgatgactctacccga
tgaatcggacaatgtactgggccatgtagaataaattatcaacgaattatcgtctacgaacatttatatcatttgttttaattt
tagtacgcgaataaatagatataaaatagaaaataacagatattacaaccaatgttatggccgcgcccggccggcc
gcggccgctttttttctagacactaaataaatagtaagattaaattaattataaaattatgtatataatattaattataaaatt
atgtatatgatttactaactttagttagataaattaataatacataaattttagtatattaatattataaattaataatacataaa
ttttagtatattaatattatattttaaatatttatttagtgtctagaaaaaaaggcctagcaggcc

Sequence of sMVA fragment 3 (F3, 62068 bp in length) (SEQ ID NO: 14):

ggcctagcaggcctttttttctagacactaaataaatagtaagattaaattaattataaaattatgtatataatattaattata
aaattatgtatatgatttactaactttagttagataaattaataatacataaattttagtatattaatattataaattaataatac
ataaattttagtatattaatattatattttaaatatttatttagtgtctagaaaaaaaggccggccgggtattaataatatctat
atttccagcgttaagtgtaacattaaacagttttaattcacgtgacgtggtatcaattaaataattaatgcccaatttggata
tagcagcctgaagctcatcttgtttagttacggatcctaatgagttattaagcaatatatcgaacggatgaacgaaggtt
gttttaagttggtcacatactttgtaatctagacatagatgcggaagaacggtagaaactatacgaaataaatattcaga
gtcctctaattgatcaagagtaactattgacttaataggcatcatttatttagtattaaatgacgaccgtaccagtgacgga
tatacaaaacgatttaattacagagttttcagaagataattatccatctaacaaaaattatgaaataactcttcgtcaaat
gtctattctaactcacgttaacaacgtggtagatagagaacataatgccgccgtagtgtcatctccagaggaaatatcc
tcacaacttaatgaagatctatttccagatgatgattctccggccactattatcgaacgagtacaacctcatactactatta
ttgacgatactccacctcctacgtttcgtagagagttattgatatcggaacaacgtcaacaacgagaaaaaagatttaa
tattacagtatcgaaaaatgctgaagcaataatggaatctagatctatgatatcttctatgccaacacaaacaccatcct
tgggagtagtttatgataaagataaaagaattcagatgttggaggatgaagtggttaatcttagaaatcaacgatctaat
acaaaatcatctgataatttagataattttaccagaatactatttggtaagactccgtataaatcaacagaagttaataag
cgtatagccatcgttaattatgcaaatttgaacgggtctcccttatcagtcgaggacttggatgtttgttcagaggatgaaa
tagatagaatctataaaacgattaaacaatatcacgaaagtagaaaacgaaaaattatcgtcactaacgtgattattat
tgtcataaatattatcgagcaagcattgctaaaactcggatttgaagaaatcaaaggactgagtaccgatatcacttca
gaaattatcgatgtggagatcggagatgactgcgatgctgtagcatcaaaactaggaatcggtaacagtccggttctt
aatattgtattgtttatactcaagatattcgttaaacgaattaaaattatttaatttaatacattcccatatccagacaacaat
cgtctggattaatctgttcctgtcgtctcataccggacgacatattaatctttttattagtaggcatctttttagatggtttctttttc
ccagcattaactgagtcgatacctagaagatcgtgattgatctctccgaccattccacgaacttctaattggccgtctctg
acggtaccataaactattttaccagcattagtaacagcttggacaatctgaccatccatcgcattgtacgatgtagtagt
aactgttgttctacgtctaggagcaccagaagtatttttggagcccttggatgttgatgtagaagaagacgaggattttga
ttttggtttacatgtaatacattttgtatcacatgcgccggcagtcacatctgtttgagaattaagattattgttgcctcctttga
cggctgcatctccaccgatttgcgctagtagatttttaagctgtggtgtaatcttattaactgtttcgatataatcatcgtaact
gcttctaacggctaaattttttttatccgccatttagaagctaaaaatatttttatttatgcagaagatttaactagattataca
atgaactaatatgatccttttccagattatttacaaacttggtattttttggttctggaggaggcgaatttaaattcggacttgg
atttggattttgtgggttcttgatcttattatacagcgcatataggatggcgacggtaactgctacgcaaataccgatcaac
aaaagaataccaatcatttattgacaataacttcactattgatcaagtatgcaatatatcatcttttcactaaataagtagt
aataatgattcaacaatgtcgagatatatggacgataataatttagttcatggaaatatcgctatgattggtatgaatgac
tccgctaactctgtggggcgcgcagtgctttccccacatagaataaattagcattccgactgtgataataataccaagta
taaacgccataatactcaatactttccatgtacgagtgggactggtagacttactaaagtcaataaaggcgaagatac
acgaaagaatcaaaagaatgattccagcgattagcacgccggaaaaataatttccaatcataagcatcatatccattt
aactaataaaaattttaaatcgccgaatgaacaaagtggaatataaaccatataaaaacaatagtttgtactgcaaaa
ataatatctatttttgttttcgaagatatggtaaaattaaatagtagtacacagcatgttataactaacagcagcaacggct
cgtaattacttatcatttactagacgaaaaggtggtgggatattttcttgctcaaataatacgaatatatcacccatccatttt
atgcgatgtttatatactctaatctttaatagatctatagacgacgggtttaccaacaatatagattttatcgattcatctaatt
taaacccttccttaaacgtgaatgatctattatctggcataacgatgactctacccgatgaatcggacaatgtactgggc
catgtagaataaattatcaacgaattatcgtctacgaacatttatatcatttgttttaattttagtacgcgaataaatagatat
aaaatagaaaataacagatattacaaccaatgttatggccgcgcccaaccaggtaggcagttttattttatcttttactac
aggttctcctggatgtacgtcaccaacggcggacgtagttctagtacaattagacgtaagttccgcttgggaattttttaa
cgctaaagagttaacgttaatcgtgcacccaacgtatttacatctagttctttgaacatcttgattataatataaccattttct
atctctagattcgtcggtgcactcatgtaaccaacataccctaggtcctaaatatttatctccggaattagattttggataat
tcgcgcaccaacaatttctatttcctttatgatcgttacaaaagacgtataatgccgtatccccaaaagtaaaataatcag
gacgaataattctaataaactcagaacaatatctcgcatccatatgtttggagcaaatatcggaataagtagacatagc
cggtttccgttttgcacgtaaccattctaaacaattggggtttccaggatcgtttctacaaaatccagtcatgaaatcatca
caatgttctgtcttgtaattattattaaatatttttggacagtgtttggtatttgtcttagaacaacattttgccacgctatcactat
cgcccaggagataatccttttttataaaatgacatcgttgcccggatgctatataatcagtagcgtgttttaaatccttaata
tattcaggagttacctcgttctgataatagattaatgatccaggacgaaatttgaaagaactacatggttctccatgaatt
aatacatattgtttagcaaattcaggaactataaaactactacaatgatctatcgacataccatctatcaaacaaaactt
gggtttaatttctcccggagatgtttcataatagtacgtataactttcttctgcaaacttaacagctctattatattcaggataa
ttaaaacctaattccatatatttgtctcgtatatctgctattcctggtgctattttgattctattaagagtaacggctgcccccatt
tttaataatcgtcagtatttaaactgttaaatgttggtatatcaacatttaccttatttcccgcagtataaggtttgttgcaggta
tactgttcaggaatggttacatttatactttttctatagtcctgtctttcgatgttcatcacatatgcaaagaacagaataaac
aaaataatgtaagaaataatattaaatatctgtgaattcgtaaatacattgattgccataataattacagcagctacaata
catacaatagacattcccacagtgttgccattacctccacgatacatttgagttactaagcaataggtaataactaagct
agtaagaggcaatagaaaagatgagataaatatcatcaatatagagattagaggagggctatatagagccaagac
gaacaaaatcaaaccgagtaacgttctaacatcattatttttgaagattcccaaataatcattcattcctccataatcgtttt
gcatcatacctccatctttaggcataaacgattgctgctgttcctctgtaaataaatctttatcaagcactccagcacccgc
agagaagtcgtcaagcatattgtaatatcttaaataactcatttatatattaaaaaatgtcactattaaagatggagtata
atctttatgccgaactaaaaaaaatgacttgtggtcaacccctaagtctttttaacgaagacggggatttcgtagaagttg
aaccgggatcatcctttaagtttctgatacctaagggattttacgcctctccttccgtaaagacgagtctagtatttgaaac
attaacaacgaccgataataaaattactagtatcaatccaacaaatgcgccaaagttatatcctcttcaacgcaaagtc
gtatctgaagtagtttctaatatgaggaaaatgatcgaatcaaaacgtcctctatacatcactcttcacttggcgtgtggat
ttggtaagactattaccacgtgttatcttatggctacacacggtagaaaaaccgtcatttgcgtacccaataaaatgttaa
tacatcaatggaagacacaggtagaggcagtcggattggaacataagatatccatagatggagtaagtagtctatta
aaggaactaaagactcaaagtccggatgtattaatagtagtcagtagacatctgacaaacgatgccttttgtaaatata
tcaataagcattatgatttgttcatcttggatgaatcacatacgtataatctgatgaacaatacagcagttacaagattttta
gcgtattatcctccgatgatgtgttattttttaactgctacacctagaccatctaacagaatttattgtaacagtattattaatat
tgccaagttatccgatctaaaaaaaactatctatgcagtagatagtttttttgagccatattccacagataatattagacat
atgataaaacgattagatggaccatctaataaatatcatatatataccgagaagttattatctgtagacgagcctagaa
atcaacttattcttaataccctggtagaagaattcaagtcaggaactattaatcgcattttagttattactaaactacgtgaa
catatggtattcttctacaaacgattattagattttttcggatcagaggttgtatttataggagacgcccaaaatagacgta
ctccagatatggtcaaatcaatcaaggaactaaatagatttatattcgtatccaccttattttattccggtactggtttagata
ttcctagtttggattctttgttcatttgctcggcagtaatcaacaatatgcaaatagagcaattactagggagggtatgtcg
agaaacagaactattagataggacggtatatgtatttcctagcacatccatcaaagaaataaagtacatgataggaa
atttcgttcaacgaattattagtctgtctgtagataaactaggatttaaacaaaaaagttatcggaaacatcaagaatcc
gatcccacttctgtatgtacaacatcctccagagaagaacgtgtattaaatagaatatttaactcgcaaaatcgttaaga
agtttaagcgacgatccgcatgctgcgcaggccagtgtattacccctcatagtattaatataatccaatgatacttttgtg
atgtcggaaatcttaaccaatttagactgacaggcagaacacgtcatgcaatcatcatcgtcatcgataactgtagtctt
gggcttctttttgcggctcttcattccggaacgcacattggtgctatccatttaggtagtaaaaaataagtcagaatatgcc
ctatagcacgatcgtgcaaaacctggtatatcgtctctatctttatcacaatatagtgtatcgacatctttattattattgacct
cgtttatcttggaacatggaatgggaacatttttgttatcaacggccacctttgccttaattccagatgttgtaaaattataac
taaacagtctatcatcgacacaaatgaaattcttgtttagacgtttgtagtttacgtatgcggctcgttcgcgtctcattttttc
agatattgcaggtactataatattaaaaataagaatgaaataacataggattaaaaataaagttatcatgacttctagc
gctgatttaactaacttaaaagaattacttagtctgtacaaaagtttgaaattttcagattctgcggctatagaaaagtata
attctttggtagaatggggaacatctacttactggaaaataggcgtgcaaaaggtagctaatgtcgagacgtcaatatc
tgattattatgatgaggtaaaaaataaaccgtttaatattgatccgggctattacattttcttaccggtatattttgggagcgt
ctttatttattcgaagggtaaaaatatggtagaacttggatctggaaactcttttcaaataccagatgatatgcgaagtgc
gtgtaacaaagtattagacagcgataacggaatagactttctgagatttgttttgttaaacaatagatggataatggaag
atgctatatcaaaatatcagtctccagttaatatatttaaactagctagtgagtacggattaaacatacccaaatatttag
aaattgaaatagaggaagacacattatttgacgacgagttatactctattatagaacgctctttcgatgataaatttccaa
aaatatccatatcgtatattaagttgggagaacttaggcggcaagttgtagactttttcaaattctcattcatgtatattgagt
ccatcaaggtagatcgtataggagataatatttttattcctagcgttataacaaaatcaggaaaaaagatattagtaaaa
gatgtagaccatttaatacgatccaaggttagagaacatacatttgtaaaagtaaaaaagaaaaacacattttccatttt
atacgactatgatggaaacggaacagaaactagaggagaagtaataaaacgaattatagacactataggacgag
actattatgttaacggaaagtatttctctaaggttggtagtgcaggcttaaagcaattgactaataaattagatattaatga
gtgcgcaactgtcgatgagttagttgatgagattaataaatccggaactgtaaaacgaaaaataaaaaaccaatcag
catttgatttaagcagagaatgtttgggatatccagaagcggattttataacgttagttaataacatgcggttcaaaatag
aaaattgtaaggttgtaaatttcaatattgaaaatactaattgtttaaataacccgagtattgagactatatatggaaacttt
aaccagttcgtctcaatctttaatatcgtcaccgatgtcaaaaaaagattattcgagtgaaataatatgcgcctttgatata
ggtgcaaaaaatcctgccagaactgttttagaagtcaaggataactccgttagggtattggatatatcaaaattagact
ggagttctgattgggaaaggcacatagctaaagatttgtcacaatatgaatacactacagttcttctagaacgtcagcct
agaaggtcgccgtacgtcaaatttatctattttattaaaggctttttatatcatacatcggctgccaaagttatttgcgtctcg
cctgtcatgtctggtaattcatatagagatcgaaaaaagagatcggtcgaagcatttcttgattggatggacacattcgg
attgcgagactccgttccggatagacgcaaattagacgatgtagcggatagtttcaatttggctatgagatacgtattag
ataaatggaatactaattatacaccttataataggtgtaaatctagaaattacataaaaaaaatgtaataacgttagtaa
cgccattatggataatctatttacctttctacatgaaatagaagatagatatgccagaactatttttaactttcatctaataag
ttgcgatgaaataggagatatatatggtcttatgaaagaacgcatttcctcagaggatatgtttgataatatagtgtataat
aaagatatacatcatgccattaagaaactagtgtattgcgacatccaacttactaaacacattattaatcagaatacgta
tccggtatttaacgattcttcacaagtgaaatgttgtcattatttcgatataaactcagataatagcaatattagctctcgtac
agtagagatatttgagagggaaaagtcatctcttgtatcatatattaaaactaccaataagaagagaaaggtcaattat
ggggaaataaagaaaactgtacatggaggcactaatgcaaattacttttccggtaaaaagtctgatgagtatctgagc
actacagtcaggtccaacattaatcaaccttggatcaaaaccatttctaagagaatgagagtagatatcattaatcact
ctatagtaacgcgtggaaaaagctctatattacaaactatagaaattatttttactaatagaacatgtgtgaaaatattca
aggattctactatgcacattattctatccaaggacaaggatgaaaaggggtgtatacacatgattgacaaattattctat
gtctattataatttatttctgttgttcgaggatatcatccaaaacgagtactttaaagaagtagctaatgttgtaaaccacgt
actcacggctacggcattagatgagaaattattcctaattaagaaaatggctgaacacgatgtttatggagttagcaatt
tcaaaatagggatgtttaacctgacatttattaagtcgttggatcataccgttttcccctctctgttagatgaggatagcaaa
ataaagttttttaaggggaaaaagctcaatattgtagcattacgatctctggaggattgtataaattacgtgactaaatcc
gagaatatgatagaaatgatgaaggaaagatcgactattttaaatagcatagatatagaaacggaatcggtagatcg
tctaaaagaattgcttctaaaatgaaaaaaaaacactaattcagaaatggatcaacgactcggatataagtttttggtg
cctgatcctaaagccggagttttttatagaccgttacatttccaatatgtatcgtattctaattttatattgcatcgattgcatga
aatcttgaccgtcaagcggccactcttatcgtttaagaataatacagaacgaattatgatagaaattagcaatgttaaa
gtgactcctccagattactcacctataatcgcgagtattaaaggtaagagttatgacgcattagccacgttcactgtaaa
tatctttaaagaggtaatgaccaaagagggtatatccatcactaaaataagtagttatgagggaaaagattctcatttga
taaaaattccgctactaataggatacgggaataaaaatccacttgatacagccaagtatcttgttcctaatgtcataggt
ggagtctttatcaataaacaatctgtcgaaaaagtaggaattaatctagtagaaaagattacaacatggccaaaattta
gggttgttaagccaaactcattcactttctcgttttcctccgtatcccctcctaatgtattaccgacaagatatcgccattaca
agatatctctggatatatcacaattggaagcgttgaatatatcatcgacaaagacatttataacggtcaatattgttttgct
gtctcaatatttatctagagtgagtctagaattcattagacgtagtttatcatacgatatgcctccagaagttgtctatctagt
aaacgcgataatagatagtgctaaacgaattactgaatctattactgactttaatattgatacatacattaatgacctggt
ggaagctgaacacattaaacaaaaatctcagttaacgatcaacgagttcaaatatgaaatgctgcataactttttacct
catatgaactatacacccgatcaactaaagggattttatatgatatctttactaagaaagtttctctactgtatctaccaca
cttctagatatccagatagagattcgatggtttgtcatcgcatcctaacgtacggcaaatattttgagacgttggcacatg
atgaattagagaattacataggcaacatccgaaacgatatcatgaacaatcacaagaacagaggcacttacgcggt
aaacattcatgtactaacaactcctggacttaatcatgcattttctagtctattgagtggaaagttcaaaaagtcagacgg
tagttatcgaacacatcctcactattcatggatgcagaatatttctattcctaggagtgttggattttatccggatcaagtaa
agatttcaaagatgttttctgtcagaaaataccatccaagtcaatatctttacttttgttcatcggacgttccggaaagaggt
cctcaggtaggtttagtatctcaattgtctgtcttgagttccattacaaatatactaacgtctgagtatttggatttggaaaag
aaaatttgtgagtatatcagatcatattataaagatgatataagttactttgaaacaggatttccaatcactatagaaaat
gctctagtcgcatctcttaatccaaatatgatatgtgattttgtaactgactttagacgtagaaaacggatgggattcttcgg
taacttggaggtaggtattactttagttagggatcacatgaatgaaattcgcattaatattggagcgggaagattagtca
gaccattcttggttgtggataacggagagctcatgatggatgtgtgtccggagttagaaagcagattagacgacatga
cattctctgacattcagaaagagtttccgcatgtcatcgaaatggtagatatagaacaatttacttttagtaacgtatgtga
atcggttcaaaaatttagaatgatgtcaaaggatgaaagaaagcaatacgatttatgtgactttcctgccgaatttagag
atggatatgtagcatcttcactagtgggaatcaatcacaattctggacccagagctattcttggatgtgctcaagctaaa
caagctatctcttgtctgagctcggatatacgaaataaaatagacaatggaattcatttgatgtatccagagaggccaat
cgtgattagtaaggctttagaaacttcaaagattgcggctaattgcttcggccaacatgttactatagcattaatgtcgtac
aaaggtatcaatcaagaggatggaattatcatcaaaaaacaatttattcagagaggcggtctcgatattgttacagcc
aagaaacatcaagtagaaattccgttggaaaactttaataacaaagaaagagataggtctaacgcctattcaaaatt
agaaagtaatggattagttagactgaatgctttcttggaatccggagacgctatagcacgaaatatctcatcaagaact
cttgaagatgattttgctagagataatcagattagctttgatgtttccgaaaaatataccgatatgtacaaatctcgcgttg
aacgagtacaagtagaacttactgacaaagttaaggtacgagtattaaccatgaaagaaagaagacccattctagg
agacaaatttaccactagaacgagtcaaaagggaacagtcgcgtatatcgcggatgaaacggaacttccatacgat
gaaaatggtatcacaccagatgtcattattaattctacatccatcttctctagaaaaactatatctatgttgatagaggttatt
ttaacagccgcatattctgctaagccgtacaacaataagggagaaaaccgacctgtctgttttcctagtagtaacgaa
acatccatcgatacatatatgcaattcgctaaacaatgttatgagcattcaaatccgaaattgtctgatgaagaattatcg
gataaaatcttttgtgaaaagattctctatgatcctgaaacggataagccttatgcatccaaagtattttttggaccaattta
ttacttgcgtctgaggcatttaactcaggacaaggcaaccgttagatgtagaggtaaaaagacgaagctcattagaca
ggcgaatgagggacgaaaacgtggaggaggtatcaagttcggagaaatggagagagactgtttaatagcgcatg
gtgcagccaatactattacagaagttttgaaagattcggaagaagattatcaagatgtgtatgtttgtgaaaattgtgga
gacatagcagcacaaatcaagggtattaatacatgtcttagatgttcaaaacttaatctctctcctctcttaacaaaaatt
gataccacgcacgtatctaaagtatttcttactcaaatgaacgccagaggcgtaaaagttaaattagatttcgaacgaa
ggcctccttcgttttataaaccattagataaagttgatctcaaaccgtcttttctggtgtaatattctagtttggtagtagatac
atatcaatatcatcaaattcgagatccgaattataaaatgggcgtggattgttaactatagaatcggacgtctgatattcg
aaaatctgtggagttttaggttttggtggaggtgtaactgctacttgggatactgaagtctgatattcagaaagctggggg
atgttctggttcgacatccaccgatggtgtcacatcactaatcggttcggtaacgtctgtggacgatggaggcaccactt
ctacaggttctggttctttatcctcagtcatcaacggagctacttcaatgcgaggaaatgtataatttggtaatggtttctcat
gtggatctgaagaagaggtaagatatctactagaaagataccgatcacgttctagttctcttttgtagaacttaactttttct
ttctccgcatctagttgatattccaacctcttcacgttcgcatgggttacctccgcagtttttacgagcgatttcacgttcagc
cttcatgcgtcttatagcatgaattcgcttatcgttatcgggtttagcttctgtcaccttagcaattccttttttattaaactctaca
taatcatatccatttctattgtttgttctaatataaacgagtatagcatcattgctaaatttttcaatagtatcgaaaacagaat
atcctaaaccatataatatatattcaggaacactcaaactaaatgtccaggattctcctaaatacgtaaactttaatagtg
cgaaatcattcaaaaatctaccacttatagatagatagatagtacataaatgcgtatagtagtctacctatctctttattatg
aaaaccggcattacgatcatatatgtcgtgatatacctgtgatccgtttacgttaaaccataaatacatgggtgatcctat
aaacatgaatttatttctaattctcagagctatagttaattgaccgtgtaatatttgcttacatgcatacttgatacgatcatta
ataagatttttatcattgctcgttatttcagaatcgtatatataaggagtaccatcgtgattcttaccagatattatacaaaat
actatatataaaatatattgacccacgttagtaatcatgtaaatgtttaacgttttaaattttgtattcaatgatccattatcata
cgctagcatggtcttatgatattcattctttaaaatataatattgtgttagccattgcattggggctcctaatggagattttttatt
ctcatccattttaggataggctttcataaagtccctaataacttcgtgaataatgtttctatgttttctactgatgcatgtatttgc
ttcgatttttttatcccatgtttcatctatcatagatttaaacgcagtaatgctcgcaacattaacatcttgaaccgttggtaca
attccgttccataaatttataatgttcgccatttatataactcattttttgaatatacttttaattaacaaaagagttaagttactc
atatggacgccgtccagtctgaacatcaatctttttagccagagatatcatagccgctcttagagtttcagcgtgattttcc
aacctaaatagaacttcatcgttgcgtttacaacacttttctatttgttcaaactttgttgttacattagtaatctttttttccaaatt
agttagccgttgtttgagagtttcctcattgtcgtcttcatcggctttaacaattgcttcgcgtttagcctctggctttttagcagc
cgttgtagaaaaaaattcagttgctggaattgcaagatcgtcatctccggggaaaagagttccgtccatttaaagtaca
gattttagaaactgacactctgcgttatttatatttggtacaacacatggattataaatattgatgttaataacatcagaaaa
tgtaaagtctatacattgttgcatcgtgttaaattttctaatggatctagtattattgggtccaacttctgcctgaaatccaaat
atggaagcggatacaaaaccgtttcctggataaaccacacatctccacttttgctttacatcagaaattgtgtcgttgaca
tcttgaactctcctatctaatgccggtgttccacctatagattttgaatattcgaatgctgcatgagtagcattaaattcctta
atattgccataattttcatatattgagtaaccctggataaaaagtaaacacaccgcagccgtagctaccacaataaaa
aaaattgatagagagttcatttataatctattagaagctgacaaaatttttttacacgcatcagacaatgctttaataaata
gttcaacatctacttttgtcatatcgaaccgatggtatgattctaacctagaattacatccgaaaaagttgactatgttcata
gtcattaagtcattaacaaacaacattccagactctggattataagacgatactgtttcgtcacaattacctaccttaatca
tgtgattatgaatattggctattagagcaccttctaagaaatctataatatctttgaaacacgatttaaaatcaaaccacg
aatatacttctacgaagaaagttagtttacccataggagaaataactataaatggagatctaaatacaaaatccggat
ctatgatagttttaacattattatattctctattaaatacctccacatctaaaaatgttaattttgaaactatgtcttcgtttattac
cgtacctgaactaaacgctataagctctattgtttgagaactctttaaacgatattcttgaaatacatgtaacaaagtttcct
ttaactcggtcggtttatctaccatagttacagaatttgtatccttatctataatataataatcaaaatcgtataaagttatata
attatcgcgttcagattgggatcttttcaaatagactaaaaaccccatttctctagtaagtatcttatgtatatgtttgtaaaat
atcttcatggtgggaatatgctctaccgcagttagccattcctcattgacagcggtagatgtattagacaaaactattcca
atgtttaacaagggccattttacgagattattaaatccttgtttgataaatgtagccaatgagggttcgagttcaacgacg
attgaattctcttcccgcggatgctgcatgatgaacgacgggatgttgttcgattgatttggaattctttttcgactttttgtttat
attaaatattttaaaatttatagcggatagcaattcatgtaccacggataatgtagacgcgtattgcgcatcgatatctttat
tattagataaatttatcaataaatgtgagaagtttgcctcgttaaggtcttccatttaaatattatataaacatttgtgtttgtatc
ttattcgtcttttatggaatagttttttactagtaaagctgcaattacacactttgtccgtaaaacataaatataaacaccagc
ttttatcaatcgttccaaaaagtcgacggcggacatttttaacatggcatctattttaaatacacttaggtttttggaaaaaa
catcattttataattgtaacgattcaataactaaagaaaagattaagattaaacataagggaatgtcatttgtattttataa
gccaaagcattctaccgttgttaaatacttgtctggaggaggtatatatcatgatgatttggttgtattggggaaggtaaca
attaatgatctaaagatgatgctattttacatggatttatcatatcatggagtgacaagtagtggaacaatttacaaattgg
gatcgtctatcgatagactttctctaaataggactattgttacaaaagttaataattataattatgatacattttttgacgatga
tgattgatcgctattgcacaattttgtttttttactttctaatatagcgtttagattctttttcatgtgcgaatattgatttactaaaat
atctatgtttaacttttgttctataacgtccttatcggcggtatcggtacatatacgtaattcaccttcacaaaatacggagtc
ttcgataataatagccaatcgattattggatctagctgtctgtatcatattcaacatgtttaatatatcctttcgtttcccctttac
aggcatcgatcgtagcatattttccgcgtctgagatggaaatgttaaaactacaaaaatgcgtaatgttagcccgtccta
atattggtacgtgtctataagtttggcatagtagaataatagacgtgtttaaatgccttccaaagtttaagaattctattaga
gtattgcattttgatagtttatcgcctacatcatcaaaaataagtaaaaagtgtgctgattttttatgattttgtgcgacagca
atacatttttctatgttacttttagttcgtatcagattatattctagagattcctgactactaacgaaattaatatgatttggcca
aatgtatccatcataatctggattataaacgggtgtaaacaagaatacatgtttatattttttaactagtgtagaaaacaga
gatagtaaatagatagtttttccagatccagatcctcccgttaaaaccattctaaacggcatttttaataaattttctcttgaa
aattgtttttcttggaaacaattcataattatatttacagttactaaattaatttgataataaatcaaaatatggaaaactaag
gtcgttagtagggaggagaacaaagaaggcacatcgtgacataaataacatttattatcatgatgacaccagaaaa
cgacgaagagcagacatctgtgttctccgctactgtttacagagacaaaattcagggaaagaataaacgcaaacgc
gtgattggtctatgtattagaatatctatggttatttcactactatctatgattaccatgtccgcgtttctcatagtgcgcctaaa
tcaatgcatgtctgctaacgaggctgctattactgacgccgctgttgccgttgctgctgcatcatctactcatagaaaggtt
gcgtctagcactacgcaatatgatcacaaagaaagctgtaatggtttatattaccagggttcttgttatatattacattcag
actaccagttattctcggatgctaaagcaaattgcactgcggaatcatcaacactacccaataaatccgatgtcttgact
acctggctcattgattatgttaaggatacatggggatctgatggtaatccaattacaaaaactacatccgattatcaagat
tctgatgtatcacaagaagttagaaagtatttttgtgttaaaacaatgaactaatatttatttttgtacattaataaatgaaat
cgcttaatagacaaactgtaagtaggtttaagaagttgtcggtgccggtcgctataatgatgatactctcaaccattatta
gtggcataggaacatttctgcattacaaagaagaactgatgcctagtgcttgcgccaatggatggatacaatacgata
aacattgttatttagatactaacattaaaatgtctacagataatgcggtttatcagtgtcgtaaattacgagccagattgcc
tagaccggatactagacatctgagagtattgtttagtattttttataaagattattgggtaagtttaaaaaagaccaatgat
aaatggttagatattaataatgataaagatatagatattagtaaattaacaaattttaaacaactaaacagtacgacgg
atgctgaagcgtgttatatatacaagtctggaaaactggttaaaacagtatgtaaaagtactcaatctgtactatgtgtta
aaaaattctacaagtgacaacaaaaaatgaattaataataagtcgttaacgtacgccgccatggacgccgcgtttgtt
attactccaatgggtgtgttgactataacagatacattgtatgatgatctcgatatctcaatcatggactttataggaccata
cattataggtaacataaaaactgtccaaatagatgtacgggatataaaatattccgacatgcaaaaatgctactttagc
tataagggtaaaatagttcctcaggattctaatgatttggctagattcaacatttatagcatttgtgccgcatacagatcaa
aaaataccatcatcatagcatgcgactatgatatcatgttagatatagaagataaacatcagccattttatctattcccat
ctattgatgtttttaacgctacaatcatagaagcgtataacctgtatacagctggagattatcatctaatcatcaatccttca
gataatctgaaaatgaaattgtcgtttaattcttcattctgcatatcagacggcaatggatggatcataattgatgggaaat
gcaatagtaattttttatcataaaagttgtaaagtaaataataaaacaataaatattgaactagtagtacgtatattgagc
aatcagaaatgatgctggtacctcttatcacggtgaccgtagttgcgggaacaatattagtatgttatatattatatatttgt
aggaaaaagatacgtactgtctataatgacaataaaattatcatgacaaaattaaaaaagataaagagttctaattcc
agcaaatctagtaaatcaactgatagcgaatcagactgggaggatcactgtagtgctatggaacaaaacaatgacg
tagataatatttctaggaatgagatattggacgatgatagcttcgctggtagtttaatatgggataacgaatccaatgttat
ggcgcctagcacagaacacatttacgatagtgttgctggaagcacgctgctaataaataatgatcgtaatgaacaga
ctatttatcagaacactacagtagtacttaatgaagataccaaacagaatcctaactattcatccaatcctttcgtaaatt
ataataaaaccagtatttgtagcaagtcaaatccgttcattacagaactcaacaataaatttagtgagaataatccgttt
agacgagcacatagcgatgattatcttaataagcaagaacaagatcatgaacacgatgatatagaatcattggtgtg
attagtttcctttttataaaattgaagtaatatttagtattattgctgccgtcacgttgtacaaatggagatattccctgtattcg
gcatttctaaaattagcaattttattgctaataatgactgtagatattatatagatacagaacatcaaaaaattatatctgat
gagatcaatagacagatggatgaaacggtacttcttaccaacatcttaagcgtagaagttgtaaatgacaatgagatg
taccatcttattcctcatagattatcgacgattatactctgtattagttctgtcggaggatgtgttatctctatagataatgacgt
caatggcaaaaatattctaacctttcccattgatcatgctgtaatcatatccccactgagtaaatgtgtcgtagttagcaa
gggtcctacaaccatattggttgttaaagcggatatacctagcaaacgattggtaacatcgtttacaaacgacatactgt
atgtaaacaatctatcactgattaattattcgccgttgtctgtattcattattagacgagttaccgactatttggatagacaca
tatgcgatcagatatttgcgaataataagtggtattccattataaccatcgacaataagcagtttcctattccatcaaactg
tataggtatgtcctctgccaagtacataaattctagcatcgagcaagatactttaatacatgtttgtaacctcgagcatcc
attcgacttagtatacaaaaaaatgcagtcgtacaattctgtacctatcaaggaacaaatattgtacggtagaattgata
atataaatatgagcattagtatttctgtggattaatagatttctagtatggggatcattaatcatctctaatctctaaatacctc
ataaaacgaaaaaaaagctattatcaaatactgtacggaatggattcattctcttctctttttatgaaactctgttgtatatct
actgataaaactggaagcaaaaaatctgataaaaagaataagaataagatcaaggattattataaaataacaatag
ttcctggttcctcttccacgtctactagctcgtggtattatacacatgcctagtaatagtctctttgcgttgacggaaagcag
actagaaataacaggctaaaatgttcagacaccataatagttcccaacccagataataacagagtaccatcaacac
attcctttaaactcaatcccaaacccaaaaccgttaaaatgtatccggccaattgatagtagataatgaggtgtacagc
gcatgatgatttacacagtaaccaaaatgaaaatactttagtaattataagaaatatagatggtaacgtcatcatcaac
aatccaataatatgccggagagtaaacattgacggataaaacaaaaatgctccgcataactctatcatggcaataac
acaaccaaatacttgtaagattcctaaattagtagaaaatacaacggatatcgatgtataagtgatctcgagaaataat
aagaataaagtaatgcccgtaaagataaacatcaacattgtttggtaatcattaaaccaattagtatgaagttgaacta
atttcacagtagattttattccagtattatccccgcatgtataagtacctggtaagatatctttatattccataatcaatgaga
catcactatctgataacgaatgaagtctagcactagtatgccatttacttaatattgtcgtcttggaagttttattataagtta
aaatatcatggttatccaatttccatctaatatactttgtcggattatctatagtacacggaataatgatggtatcattacatg
ctgtatactctatggtctttgtagttgttataacaaccaacgtatagaggtatatcaacgatattctaactcttgacattttttatt
tatttaaaatgatacctttgttatttattttattctattttgctaacggtattgaatggcataagtttgaaacgagtgaagaaata
atttctacttacttattagacgacgtattatacacgggtgttaatggggcggtatacacattttcaaataataaactaaaca
aaactggtttaactaataataattatataacaacatctataaaagtagaggatgcggaaccaataacggaaatccca
aatgttggaaaatagacggttcagacgacccaaaacatagaggtagaggatacgctccttatcaaaatagcaaagt
aacgataatcagtcacaacggatgtgtactatctgacataaacatatcaaaagaaggaattaaacgatggagaaga
tttgacggaccatgtggttatgatttatacacggcggataacgtaattccaaaagatggtttacgaggagcattcgtcga
taaagatggtacttatgacaaagtttacattcttttcactgatactatcggctcaaagagaattgtcaaaattccgtatata
gcacaaatgtgcctaaacgacgaaggtggtccatcatcattgtctagtcatagatggtcgacgtttctcaaagtcgaatt
agaatgtgatatcgacggaagaagttatagacaaattattcattctagaactataaaaacagataatgatacgatacta
tatgtattcttcgatagtccttattccaagtccgcattatgtacctattctatgaataccattaaacaatctttttctacgtcaaa
attggaaggatatacaaagcaattgccgtctccagctcctggtatatgtttaccagctggaaaagttgttccacatacca
cgtttgaagtcatagaaaaatataatgtactagatgatattataaagcctttatctaaccaacctatcttcgaaggaccgt
ctggtgttaaatggttcgatataaaggagaaggaaaatgaacatcgggaatatagaatatacttcataaaagaaaatt
ctatatattcgttcgatacaaaatctaaacaaactcgtagctcgcaagtcgatgcgcgactattttcagtaatggtaactt
cgaaaccgttatttatagcagatatagggataggagtaggaatgccacaaatgaaaaaaatacttaaaatgtaatctt
aatcgagtacaccgcacgacaatgaacaaacataagacagattatgctggttatgcttgctgcgtaatatgcggtcta
attgttggaattatttttacagcgacactattaaaagttgtagaacgtaaattagttcatacaccatcaatagataaaacg
ataaaagatgcatatattagagaagattgtcctactgactggataagctataataataaatgtatccatttatctactgatc
gaaaaacctgggaggaaggacgtaatgcatgcaaagctctaaatccaaattcggatctaattaagatagagactcc
aaacgagttaagttttttaagaagcattagacgcggatattgggtaggagaatccgaaatattaaaccagacaaccc
catataattttatagctaaaaatgccacgaagaatggaactaaaaaacggaaatatatttgtagtacaacgaatactc
ccaaactacatttttatcataccactacttcggttagatgttttagaaaaaaataaatatcgccgtaccgttcttgtttttataa
aaataacaattaacaattatcaaattttttctttaatattttacgtggttgaccattcttggtggtaaaataatctcttagtgttgg
aatggaatgctgtttaatgtttccgcactcatcgtatattttgacgtatgcagtcacatcgtttacgcaatagtcagactgta
gttctatcatgcttcctacatcagaaggaggaacagttttaaagtctcttggttttaatctattgccattagttttcatgaaatc
ctttgttttatccacttcacattttaaataaatgtccactatacattcttctgttaattttactagatcgtcatgggtcatagaattt
ataggttccgtagtccatggatccaaactagcaaacttcgcgtatacggtatcgcgattagtgtatacaccaactgtatg
aaaattaagaaaacagtttaataaatcaacagaaatatttaatcctccgtttgatacagatgcgccatatttatggatttc
ggattcacacgttgtttgtctgaggtgttcgtctagtgttgcttctacgtaaacttcgattcccatatattctttattgtcagaatc
gcataccgatttatcatcatacactgtttgaaaactaaatggtatacacatcaaaataataaataataacgagtacattct
gcaatattgttatcgtaattggaaaaatagtgttcgagtgagttggattatgtgagtattggattgtatattttattttatattttat
attttgtagtaagaatagaatgctaatgtcaagtttattccaatagatgtcttattaaaaaacatatataataaataacaat
ggctgaatggcataaaattatcgaggatatctcaaaaaataataagttcgaggatgccgccatcgttgattacaagac
tacaaagaatgttctagctgctattcctaacagaacatttgccaagattaatcctctcatcactaatcgtaatattctaaaa
cctcttattggtcagaaatattgtattgtatatactaactctctaatggatgagaacacgtatgctatggagttgcttactgg
gtacgcccctgtatctccgatcgttatagcgagaactcataccgcacttatatttttgatgggtaagccaacaacatcca
gacgtgacgtgtatagaacgtgtagagatcacgctacccgtgtacgtgcaactggtaattaaaataaaaagtaatatt
catatgtagtgtcaattttaaatgatgatgatgaaatggataatatccatattgacgatgtcaataatgccggtattggcat
acagttcatcgatttttagatttcattcagaggatgtggaattatgttatgggcatttgtattttgataggatctataatgtagta
aatataaaatataatccgcatattccatatagatataattttattaatcgcacgttaaccgtagatgaactagacgataat
gtcttttttacacatggttattttttaaaacacaaatatggttcacttaatcctagtttgattgtctcattatcaggaaacttaaa
atataatgatatacaatgctcagtaaatgtatcgtgtctcattaaaaatttggcaacgagtacatctactatattaacatct
aaacataagacttattctctacatcggtccacgtgtattactataataggatacgattctattatatggtataaagatataa
atgacatctatgattttactgcaatatgtatgctaatagcgtctacattgatagtgaccatatacgtgtttaaaaaaataaa
aatgaactcttaattatgctatgctattagaaatggataaaatcaaaattacggttgattcaaaaattggtaatgttgttacc
atatcgtataacttggaaaagataactattgatgtcacacctaaaaagaaaaaagaaaaggatgtattattagcgcaa
tcagttgctgtcgaagaggcaaaagatgtcaaggtagaagaaaaaaatattatcgatattgaagatgacgatgatat
ggatgtagaaagcgcataatacgatctataaaaataagtatataaatactttttatttactgtactcttactgtgtagtggtg
ataccctactcgattatttttttaaaaaaaaatacttattctgattcttctagccatttccgtgttcgttcgaatgccacatcgac
gttaaagataggggagtagttgaaatctagttctgcattgttggtacgcacctcaaatgtagtgttggatatcttcaacgta
tagttgttgagtagtgatggttttctaaatagaattctcttcatatcattcttgcacgcgtacatttttagcatccatcttggaatc
ctagatccttgttctattcccaatggtttcatcaatagaagattaaacatatcgtacgaacacgatggagagtaatcgtag
caaaagtaagcatttcctttaatctcagatcccggatactggatatattttgcagccaacacgtgcatccatgcagcattt
cctacatatacccggctatgtaccgcgttatcatcgactgtacgatacataatgttaccgtgttgcttacattgctcgtaaa
agactttcatcaatttgtctccttctccgtaaattccagtgggtcttaggcaacaagtatacaattttgctccattcatgattac
ggaattattggctttcataaccagttgctcggccatacgtttactttttgcgtatacatgtcctggtgatatatcataaagggt
atgctcatggccgatgaatggatcaccgtgtttattgggtcctattgcttccatgctactagtatagatcaaatacttgattc
ctaggtccacacaagctgccaatatagtctgtgttccataatagtttactttcatgatttcattatcggtgtattttccaaatac
atccactagagcagctgtatgaataatcagatttaccccatctagcgcttctcttaccttatcaaagtcgtttatatcacatt
gtatatagtttataaccttaactttcgaggttattggttgtggatcttctacaatatctatgactctgatttcttgaacatcatctg
cactaattaacagttttactatatacctgcctagaaatccggcaccaccagtaaccgcgtacacggccattgctgccac
tcataatatcagactacttattctattttactaaataatggctgtttgtataatagaccacgataatatcagaggagttattta
ctttgaaccagtccatggaaaagataaagttattggattaaaatccggaacgtatagtttgataattcatcgttacggag
atattagtcaaggatgtgattccataggcagtccagaaatatttatcggtaacatctttgtaaacagatatggtgtagcat
atgtttatttagatacagatgtaaatatatctacaattattggaaaggcgttatctatttcaaaaaatgatcagagattagc
gtgtggagttattggtatttcttacataaatgaaaagataatacattttcttacaattaacgagaatggcgtttgatatatca
gttaatgcgtctaaaacaataaatgcattagtttacttttctactcagcaaaataaattagtcatacgtaatgaagttaatg
atacacactacactgtcgaatttgatagggacaaagtagttgacacgtttatttcatataatagacataatgactccatag
agataagaggggtgcttccagaggaaactaatattggttgcgcggttaatacgccggttagtatgacttacttgtataat
aagtatagttttaaactgattttagcagaatatataagacacagaaatactatatccggcaatatttattcggcattgatga
cactagatgatttggctattaaacagtatggagacattgatctattatttaatgagaaacttaaagtagactccgattcgg
gactatttgactttgtcaactttgtaaaggatatgatatgttgtgattctagaatagtagtagctctatctagtctagtatctaa
acattgggaattgacaaataaaaaatataggtgtatggcattagccgaacatatatctgatagtattccaatatctgagc
tatctagactacgatacaatctatgtaagtatctacgcgggcacactgagagcatagaggatgaatttgattattttgaa
gacgatgattcgtctacatgttctgccgtaaccgacagggaaacggatgtataattttttttatagcgtgaaggatatgat
aaaaaatataattgttgtatttatcccattccaatcaccttatatgattctgtaacacaatgaaggagtcttatagatgtata
gaggtcagatactggtttgataaactgtttattccacataagtatgtttgactttatggttagacccgcatactttaacaaatc
actgaaaattggagttaggtattgacctctcagaatcagttgccgttctggaacattaaatgtattttttatgatatactcca
acgcatttatgtgggcatacaacaagtcattactaatggagtattccaagagaagagatttcaacagactgtttatgaa
ctcgaatgccgcctcattgtcgcttatattgatgatgtcgaattctcccaatatcatcaccgatgagtagctcatcttgttatc
gggatccaagttttctaaagatgtcattaaaccctcgatcatgaatggatttatcatcatcgtttttatgttggacatgagctt
agtccgtttgtccacatctatagacgacgatttctgaattatttcatatatccctctctttaactccaggaacttgtcaggatg
gtctactttaatatgttctcgtctaagagatgaaaatctttggatggttgcacgcgacttttctctaaaggatcctctcttaaat
gaatccatcttatccttggacaagatggacagtctattttccttagatggtttaatatttttgttacccatgatctataaaggta
gacctaatcgtctcggatgaccatatatttattttcagttttattatacgcataaattgtaaaaaatatgttaggtttacaaaa
atgtctcgtggggcattaatcgtttttgaaggattggacaaatctggaaaaacaacacaatgtatgaacatcatggaat
ctataccggcaaacacgataaaatatcttaactttcctcagagatccactgtcactggaaagatgatagatgactatct
aactcgtaaaaaaacctataatgatcatatagttaatctattattttgtgcaaatagatgggagtttgcatcttttatacaag
aacaactagaacagggaattactttaatagttgatagatacgcattctctggagtagcgtatgccgccgctaaaggcg
cgtcaatgactctcagtaagagttatgaatctggattgcctaaacccgacttagttatattcttggaatctggtagcaaag
aaattaatagaaacgtcggcgaggaaatttatgaagatgttacattccaacaaaaggtattacaagaatataaaaaa
atgattgaagaaggagatattcattggcaaattatttcttctgaattcgaggaagatgtaaagaaggagttgattaagaa
tatagttatagaggctatacacacggttactggaccagtggggcaactgtggatgtaatagtgaaattacattttttataa
atggatgaagcatattactctggcaacttggaatcagtactcggatacgtgtccgatatgcataccgaactcgcatcaa
tatctcaattagttattgccaagatagaaactatagataatgatatattaaacaaggacattgtaaattttatcatgtgtag
atcaaacttggataatccatttatctctttcctagatactgtatatactattatagatcaagagatctatcagaccgaattgat
taattcattagacgacaatgaaattatcgattgtatagttaacaagtttatgagcttttataaggataacctagaaaatata
gtagatgctatcattactctaaaatatataatgaataatccagattttaaaactacgtatgccgaagtactcggttccaga
atagcggatatagatattaaacaagtgatacgtgagaatatactacaattgtctaataatatccgcgaacgatatttgtg
aaaatattaaaaaaaaatactttttttattaaatgacgtcgcttcgcgaatttagaaaattatgctgtgatatatatcacgca
tcaggatataaagaaaaatctaaattaattagagactttataacagatagggatgataaatatttgatcattaagctattg
cttcccggattagacgatagaatttataacatgaacgataaacaaattataaaattatatagtataatatttaaacaatct
caggaagatatgctacaagatttaggatacggatatataggagacactattaggactttcttcaaagagaacacaga
aatccgtccacgagataaaagcattttaactttagaagaagtggatagttttttaactacgttatcatccgtaactaaaga
atcgcatcaaataaaattattgactgatatcgcatccgtttgtacatgtaatgatttaaaatgtgtagtcatgcttattgataa
agatctaaaaattaaagcgggccctcggtacgtacttaacgctattagtcctcatgcctatgatgtgtttagaaaatctaa
taacttgaaagagataatagaaaatgcatctaaacaaaatctagactctatatctatttctgttatgactccaattaatccc
atgttagcggaatcgtgtgattctgtcaataaggcgtttaaaaaatttccatcaggaatgtttgcggaagtcaaatacgat
ggtgaaagagtacaagttcataaaaataataacgagtttgccttctttagtagaaacatgaaaccagtactctctcata
aagtggattatctcaaagaatacataccgaaagcatttaaaaaagctacgtctatcgtattggattctgaaattgttcttgt
agacgaacataatgtaccgctcccgtttggaagtttaggtatacacaaaaagaaagaatataaaaactctaacatgt
gtttgttcgtgtttgactgtttgtactttgatggattcgatatgacggacattccattgtacgaacgaagatcttttctcaaaga
tgttatggttgaaatacccaatagaatagtattctcagagttgacgaatattagtaacgagtctcagttaactgacgtattg
gatgatgcactaacgagaaaattagaaggattggtcttaaaagatattaatggagtatacgaaccgggaaagagaa
gatggttaaaaataaagcgagactatttgaacgagggttccatggcagattctgccgatttagtagtactaggtgcttac
tatggtaaaggagcaaagggtggtatcatggcagtctttctaatgggttgttacgacgatgaatccggtaaatggaaga
cggttaccaagtgttcaggacacgatgataatacgttaagggagttgcaagaccaattaaagatgattaaaattaaca
aggatcccaaaaaaattccagagtggttagtagttaataaaatctatattcccgattttgtagtagaggatccaaaaca
atctcagatatgggaaatttcaggagcagagtttacatcttccaagtcccataccgcaaatggaatatccattagatttc
ctagatttactaggataagagaggataaaacgtggaaagaatctactcatctaaacgatttagtaaacttgactaaatc
ttaatagttacatacaaattaaaataacactatttagttggtggtcgccatggatggtgttattgtatactgtctaaacgcgtt
agtaaaacatggcgaggaaataaatcatataaaaaatgatttcatgattaaaccatgttgtgaaaaagtcaagaacg
ttcacattggcggacaatctaaaaacaatacagtgattgcagatttgccatatatggataatgcggtatccgatgtatgc
aattcactgtataaaaagaatgtatcaagaatatccagatttgctaatttgataaagatagatgacgatgacaagactc
ctactggtgtatataattattttaaacctaaagatgccattcctgttattatatccataggaaaggatagagatgtttgtgaa
ctattaatctcatctgataaagcgtgtgcgtgtatagagttaaattcatataaagtagccattcttcccatggatgtttccttttt
taccaaaggaaatgcatcattgattattctcctgtttgatttctctatcgatgcggcacctctcttaagaagtgtaaccgata
ataatgttattatatctagacaccagcgtctacatgacgagcttccgagttccaattggttcaagttttacataagtataaa
gtccgactattgttctatattatatatggttgttgatggatctgtgatgcatgcaatagctgataatagaacttacgcaaatat
tagcaaaaatatattagacaatactacaattaacgatgagtgtagatgctgttattttgaaccacagattaggattcttgat
agagatgagatgctcaatggatcatcgtgtgatatgaacagacattgtattatgatgaatttacctgatgtaggcgaattt
ggatctagtatgttggggaaatatgaacctgacatgattaagattgctctttcggtggctggtatttggaaagttttataggt
agttgatagaacaaaatacataattttgtaaaaataaatcactttttatactaatatgacacgattaccaatacttttgttact
aatatcattagtatacgctacaccttttcctcagacatctaaaaaaataggtgatgatgcaactttatcatgtaatcgaaat
aatacaaatgactacgttgttatgagtgcttggtataaggagcccaattccattattcttttagctgctaaaagcgacgtctt
gtattttgataattataccaaggataaaatatcttacgactctccatacgatgatctagttacaactatcacaattaaatcat
tgactgctagagatgccggtacttatgtatgtgcattctttatgacatcgcctacaaatgacactgataaagtagattatga
agaatactccacagagttgattgtaaatacagatagtgaatcgactatagacataatactatctggatctacacattcac
cggaaactagttctgagaaacctgattatatagataattctaattgctcgtcggtattcgaaatcgcgactccggaacca
attactgataatgtagaagatcatacagacaccgtcacatacactagtgatagcattaatacagtaagtgcatcatctg
gagaatccacaacagacgagactccggaaccaattactgataaagaagaagatcatacagttacagacactgtct
catacactacagtaagtacatcatctggaattgtcactactaaatcaaccaccgatgatgcggatctttatgatacgtac
aatgataatgatacagtaccatcaactactgtaggcggtagtacaacctctattagcaattataaaaccaaggactttgt
agaaatatttggtattaccgcattaattatattgtcggccgtggcaatattctgtattacatattatatatataataaacgttca
cgtaaatacaaaacagagaacaaagtctagatttttgacttacataaatgtctgggatagtaaaatctatcatattgagc
gggccatctggtttaggaaagacagccatagccaaaagactatgggaatatatttggatttgtggtgtcccataccact
agatttcctcgtcctatggaacgagaaggtgtcgattaccattacgttaacagagaggccatctggaagggaatagcc
gccggaacatactgagtttttaggaaatatttacggaacttctaaaactgctgtgaatacagcggctattaataatcgtat
ttgtgtgatggatctaaacatcgacggtgttagaagttttaaaaatacttacctaatgccttactcggtgtatataagacct
acctctcttaaaatggttgagaccaagcttcgttgtagaaacactgaagcggatgatgagattcatcgtcgtgtgatgttg
gcaaaaactgacatggatgaggcaggtgaagccggtctattcgacactattatcattgaagatgatgtgaatttagcat
atagtaagttaattcagatactacaggaccgtattagaatgtattttaacactaattagagacttaagacttaaaacttgat
aattaataatataactcgtttttatatgtggctatttcaacgtctaatgtattagttaaatattaaaacttaccacgtaaaactt
aaaatttaaaatgatatttcattgacagatagatcacacattatgaactttcaaggacttgtgttaactgacaattgcaaa
aatcaatgggtcgttggaccattaataggaaaaggtggatttggtagtatttatactactaatgacaataattatgtagta
aaaatagagcccaaagctaacggatcattatttaccgaacaggcattttatactagagtacttaaaccatccgttatcg
aagaatggaaaaaatctcacaatataaagcacgtaggtcttatcacgtgcaaggcatttggtctatacaaatccattaa
tgtggaatatcgattcttggtaattaatagattaggtgcagatctagatgcggtgatcagagccaataataatagattacc
aaaaaggtcggtgatgttgatcggaatcgaaatcttaaataccatacaatttatgcacgagcaaggatattctcacgg
agatattaaagcgagtaatatagtcttggatcaaatagataagaataaattatatctagtggattacggattggtttctaa
attcatgtctaatggcgaacatgttccatttataagaaatccaaataaaatggataacggtactctagaatttacacctat
agattcgcataaaggatacgttgtatctagacgtggagatctagaaacacttggatattgtatgattagatggttgggag
gtatcttgccatggactaagatatctgaaacaaagaattgtgcattagtaagtgccacaaaacagaaatatgttaaca
atactgcgactttgttaatgaccagtttgcaatatgcacctagagaattgctgcaatatattaccatggtaaactctttgac
atattttgaggaacccaattacgacaagtttcggcacatattaatgcagggtgtatattattaagtgtggtgtttggttgatgt
aaaatttttgtcgataaaaattaaaaaataacttaatttattattgatctcgtgtgtacaaccgaaatcatggcgatgttttac
gcacacgctctcggtgggtacgacgagaatcttcatgcctttcctggaatatcatcgactgttgccaatgatgtcagtttat
aataacaagtatgacattgtaaaagacaaatatatgtggtgttacagtcaggtgaacaagagatatattggagcactg
ctgcctatgtttgagtgcaatgaatatctacaaattggagatccgatccatgatcaagaaggaaatcacatatcgccac
aaaaactactatgctctaagcggaatcgggtacgagagtctagacttgtgtttggaaggagtagggattcatcatcac
gtacttgaaacagaaaacgctgtatatggaaaagttcaacatgattattctactatcaaagagaaggccaaagaaat
gaatgcactcagttcaggacctatcatcgattaccacgtctggataggagattgtatctgtcaagttactgctgtggacgt
acatggaaaggaaattatgagaatgagattcaaaaagggtgcggtgcttccgatcccaaatctggtaaaagttaaac
ttggggagaatgatacagaaaatctttcttctactatatcggcggcaccatcgaggtaaccacctctctggaagacagt
gtgaatcatgtactcatgaaacgtttggaatctatacgccatatgtggtctgttgtatatgatcattttgatattgtgaatggta
aagaatgctgttatgtgcatacgcatttgtctaatcaaaatcttataccgagtactgtaaaaacaaatttgtacatgaaga
ctatgggatcatgcattcaaatgagtatcttagcgaactgaaggaatcaggtggatggagtcccagaccagaaatgc
aggaatttgaatatccagatggagtggaagacactgaatcaattgagagattggtagaggagttcttcaatagatcag
aacttcaggctggtaaatctattaatgttaaacatacatctgtttcagctaagcaactaagaacacgtatactctcatctttt
gccaacacagagggtggatatttgttcattggagttgataataatacacacaaagtatttggattcacggtgggttacga
ctacctcagactgatagagaatgatatagaaaagcatatcaaaagactttgtgttgtgtatttctgtgagaagaaagag
gacatcaagtacacgtgtcgattcatcaaggtatataaacctggggatgaggctacctcgacatacgtgtgcgctatc
aaagtggaaagatgctgttgtgctgtgtttgcagattggccagaatcatggtatcaagaagtattctccagatgaatagg
tgtcacatataaaattttaattaatgtaactatagagaacaaataataggttgtaatatcatatagacaataactaacaat
taattagtaactgttatctctttttaactaactaactatacctattaatacatcgtaattatagttcttaacatctattaatcattga
ttcgcttctttaattttttataaaccaacattgttaattgaaaagggataacatgttacagaatataaattatatatggattttttt
taaaaaggaaatacttgactggagtgtatatttatctcttcattatatagcacgcgtgttttccaatttttccacatcccatata
atacaggattataatctcgttcgaacatacgagaaagtggataaaacaatagttgattttttatctaggttgccaaatttatt
ccatattttagaatatggggaaaatattctacatatttattctatggatgatgctaatacgaatattataattttttttctagata
gagtattaaatattaataagaacgggtcatttatacacaatctcaggttatcatcatccattaatataaaagaatatgtata
tcaattagttaataatgatcatccagataataggataagactaatgcttgaaaatggacgtagaacaagacattttttgtc
ctatatatcagatacagttaatatctatatatgtattttatatagatgccgaagacagttacggttgtacattattacatagat
gtatatatcactataagaaatcagaatcagaatcagaatcatacaatgaattaattaagatattgttaaataatggatca
gatgtagataaaaaagatacgtacggaaacacaccttttatcctattatgtaaacacgatatcaacaacgtggaattgt
ttgagatatgtttagagaatgctaatatagactctgtagactttaatagatatacacctcttcattatgtctcatgtcgtaata
aatatgattttgtaaagttattaatttctaaaggagcaaatgttaatgcgcgtaataaattcggaactactccattttattgtg
gaattatacacggtatctcgcttataaaactatatttggaatcagacacagagttagaaatagataatgaacatatagtt
cgtcatttaataatttttgatgctgttgaatctttagattatctattatccagaggagttattgatattaactatcgtactatttacg
acgctgtcagttataatgcgtataatacgttggtctatctattaaacagaaatggtgattttgagacgattactactagtgg
atgtacatgtatttcggaagcagtcgcaaacaacaacaaaataataatggaagtactattgtctaaacgaccatctttg
aaaattatgatacagtctatgatagcaattactaaacataaacagcataatgcagatttattgaaaatgtgtataaaata
tactgcgtgtatgaccgattatgatactcttatagatgtacaatcactacagcaatataaatggtatattttaagatgtttcg
atgaaatagatatcatgaagagatgttatataaaaaataaaactgtattccaattagttttttgtatcaaagacattaatac
tttaatgagatacggtaaacatccttctttcgtgaagtgcactagtctcgacgtatacggaagtcgtgtacgtaatatcata
gcatctattagatatcgtcagagattaattagtctattatccaagaagctggatcctggagataaatggtcgtgttttccta
acgaaataaaatataacgataacgaactgtccacatatctaaaaatcttataaacactattaaaatataaaatcacact
acatcattgtttccttttagtgctcgacagtgtatactatttttaacgctcataaataaaaatgaaaacgatttccgttgttacg
ttgttatgcgtactacctgctgttgtttattcaacatgtactgtacccactatgaataacgctaaattaacgtctaccgaaac
atcgtttaataataaccagaaagttacgtttacatgtgatcagggatatcattcttcggatccaaatgctgtctgcgaaac
agataaatggaaatacgaaaatccatgcaaaaaaatgtgcacagtttctgattacatctctgaactatataataaacc
gctatacgaagtgaattccaccatgacactaagttgcaacggcgaaacaaaatattttcgttgcgaagaaaaaaatg
gaaatacttcttggaatgatactgttacgtgtcctaatgcggaatgtcaacctcttcaattagaacacggatcgtgtcaac
cagttaaagaaaaatactcatttggggaatatataactatcaactgtgatgttggatatgaggttattggtgcttcgtacat
aagttgtacagctaattcttggaatgttattccatcatgtcaacaaaaatgtgatataccgtctctatctaatggattaatttc
cggatctacattttctatcggtggcgttatacatcttagttgtaaaagtggttttatactaacgggatctccatcatccacatg
tatcgacggtaaatggaatcccatactcccaacatgtgtacgatctaacgaaaaatttgatccagtggatgatggtccc
gacgatgagacagatttgagcaaactctcgaaagacgttgtacaatatgaacaagaaatagaatcgttagaagcaa
cttatcatataatcatagtggcgttaacaattatgggcgtcatatttttaatctccgttatagtattagtttgttcctgtgacaaa
aataatgaccaatataagttccataaattgctaccgtgaatataaatccgttaaaataatgaataattaataattaataatt
taataacaaacaagtatcaaaagattaaagacttatagctagaatcaattgagatgtcttcttcagtggatgttgatatct
acgatgccgttagagcatttttactcaggcactattataacaagagatttattgtgtatggaagaagtaacgccatattac
ataatatatacaggctatttacaagatgcgccgttataccgttcgatgatatagtacgtactatgccaaatgaatcacgtg
ttaaacaatgggtgatggatacacttaatggtataatgatgaatgaacgcgatgtttctgtaagcgttggcaccggaata
ctattcatggaaatgtttttcgattacaataaaaatagtatcaacaatcaactaatgtatgatataattaatagcgtatctat
aattctagctaatgagagatatagaagcgcttttaacgacgatggtatatacatccgtagaaatatgattaacaagttgt
acggatacgcatctctaactactattggcacgatcgctggaggtgtttgttattatctgttgatgcatctagttagtttgtataa
ataattatttcaatatactagttaaaattttaagattttaaatgtataaaaaactaataacgtttttatttgtaataggtgcatta
gcatcctattcgaataatgagtacactccgtttaataaactgagtgtaaaactctatatagatggagtagataatataga
aaattcatatactgatgataataatgaattggtgttaaattttaaagagtacacaatttctattattacagagtcatgcgacg
tcggatttgattccatagatatagatgttataaacgactataaaattattgatatgtctactattcaacgcagaggtcacac
gtgtagaatatctaccaaattatcatgccattatgataagtacccttatattcacaaatatgatggtgatgagcgacaata
ttctattactgcagagggaaaatgctataaaggaataaaatatgaaataagtatgatcaacgatgatactctattgaga
aaacatactcttaaaattggatctacttatatatttgatcgtcatggacatagtaatacatattattcaaaatatgatttttaaa
aatttaaaatatattatcacttcagtgacagtagtcaaataacaaacaacaccatgagatatattataattctcgcagtttt
gttcattaatagtatacacgctaaaataactagttataagtttgaatccgtcaattttgattccaaaattgaatggactggg
gatggtctatacaatatatcccttaaaaattatggcatcaagacgtggcaaacaatgtatacaaatgtaccagaagga
acatacgacatatccgcatttccaaagaatgatttcgtatctttctgggttaaatttgaacaaggcgattataaagtggaa
gagtattgtacgggaccaccgactgtaacattaactgaatacgacgaccatccgtatgctactagaggtagcaaaaa
gattcctatttacaaacgcggtgacatgtgtgatatctacttgttgtatacggctaacttcacattcggagattctaaagaa
ccagtaccatatgatatcgatgactacgattgcacgtctacaggttgcagcatagactttgtcacaacagaaaaagtgt
gcgtgacagcacagggagccacagaagggtttctcgaaaaaattactccatggagttcgaaagtatgtctgacacct
aaaaagagtgtatatacatgcgcaattagatccaaagaagatgttcccaatttcaaggacaaaatggccagagttat
caagagaaaatttaactaaatttctcggtagcacatcaaatgatgttaccacttttcttagcatgcttaacttgactaaatat
tcataactaatttttattaatgatacaaaaacgaaataaaactgcatattatacactggttaacgcccttataggctctaac
cattttcaagatgaggtccctgattatagtccttctgttcccctctatcatctactccatgtctattagacgatgtgagaagac
tgaagaggaaacatggggattgaaaatagggttgtgtataattgccaaagatttctatcccgaaagaactgattgcagt
gttcatctcccaactgcaagtgaaggcaatggattcagggatatacgaaacaccgataaattataaaaaaagcaatg
tgtccgctgtttccgttaataatactattttcgtaactggcggattattcataaataactctaatagcacgatcgtggttaaca
atatggaaaaacttgacatttataaagacaaacaatggtcgattatagaaatgcctatggctagggtatatcacggcat
cgactcgacatttggaatgttatattttgccggaggtctatccgttaccgaacaatatggtaatttagagaaaaacaacg
agatatcttgttacaatcctagaacgaataagtggtttgatatttcatatactatttataagatatccatatcatcattgtgtaa
actaaataacgtcttctatgtatttagtaaggacattggatatgtggaaaagtatgatggtctccccgctataaaggcatt
atcaacttctccttattgattgaaaatgaaaatataaatagtttttatgtatagcagtattaccctatagttttattgcttactact
aacatggatacagatacagatgttacaaatgtagaagatatcatgaatgaaatagatagagagaaagaagaaata
ctaaaaaatgtagaaattgaaaataataaaaacattaacaagaatcatcccaatgaatatattagagaagcactcgtt
attaatacaagtagtaatagtgattccattgataaagaagttatagaatgtatcagtcacgatgtaggaatatagatcat
atctactaatttttataatcaatacaaaacataaaaaacaactcgttattacatagcaggcatggaatccttcaagtattgt
tttgataacgatggcaagaaatggattatcggaaatactttatattctggtaattcaatactctataaggtcagaaaaaatt
tcactagttcgttctacaattacgtaatgaagatagatcacaaatcacacaagccattgttgtctgaaatacgattctatat
atctgtattggatcctttgactatcgacaactggacacgggaacgtggtataaagtatttggctattccagatctgtatgga
attggagaaaccgatgattatatgttcttcgttataaagaatttgggaagagtattcgccccaaaggatactgaatcagt
cttcgaagcatgcgtcactatgataaacacgttagagtttatacactctcgaggatttacccatggaaaaatagaaccg
aggaatatactgattagaaataaacgtctttcactaattgactattctagaactaacaaactatacaagagtggaaact
cacatatagattacaacgaggacatgataacttcaggaaatatcaattatatgtgtgtagacaatcatcttggagcaac
agtttcaaaacgaggagatttagaaatgttgggatattgcatgatagaatggttcggtggcaaacttccatggaaaaac
gaaagtagtataaaagtaataaaacaaaaaaaagaatataaaaaatttatagctactttctttgaggactgttttcctga
aggaaatgaacctctggaattagttagatatatagaattagtatacacgttagattattctcaaactcctaattatgacag
actacgtagactgtttatacaagattgaaatattctttttttatagagtgtggtagtgttacggatatctaatattaatattaga
ctatctctatcgcgctacacgaccaatatcgattactatggatatcttctatgaaaggagagaatgtattcatttctccagc
gtcaatctcgtcagtattgacaatactgtattatggagctaatggatccactgctgaacagctatcaaaatatgtagaaa
aggaggagaacacggataaggttagcgctcagaatatctcattcaaatccatgaataaagtatatgggcgatattctg
ccgtgtttaaagattcctttttgagaaaaattggcgataagtttcaaactgttgacttcactgattgtcgcactatagatgca
atcaacaagtgtgtagatatctttactgaggggaaaatcaatccactattggatgaacaattgtctcctagcaattagtgc
cgtatactttaaagcaaaatggttgacgccattcgaaaaggaatttaccagtgattatcccttttacgtatcaccaacgg
aaatggtagacgtaagtatgatgtctatgtacggcgagctatttaatcacgcatctgtaaaagaatcattcggtaactttt
caatcatagaactgccatatgttggagatactagtatgatggtcattcttccagacaagattgatggattagaatccata
gaacaaaatctaacagatacaaattttaagaaatggtgtaactctctggaagctacgtttatcgatgttcacattcccaa
gtttaaggtaacaggctcgtataatctggtggatactctagtaaagtcaggactgacagaggtgttcggttcaactgga
gattatagcaatatgtgtaatttagatgtgagtgtcgacgctatgatccacaaaacgtatatagatgtcaatgaagagtat
acagaagcagctgcagcaacttgtgcactggtgtcagactgtgcatcaacaattacaaatgagttctgtgtagatcatc
cgttcatctatgtgattaggcatgttgatggaaaaatttttttcgttggtagatattgctctccgacaactaattgttaaccattt
tttttaaaaaaatagaaaaaacatgtggtattagtgcaggtcgttattcttccaattgcaattggtaagatgacggccaac
tttagtacccacgtcttttcaccacagcactgtggatgtgacagactgaccagtattgatgacgtcaaacaatgtttgact
gaatatatttattggtcgtcctatgcataccgcaacaggcaatgcgctggacaattgtattccacactcctctcttttagag
atgatgcggaattagtgttcatcgacattcgcgagctggtaaaaaatatgccgtgggatgatgtcaaagattgtgtaga
aatcatccgttgttatataccggatgagcaaaaaaccatcatcggactttgtgcatatgctgctacttactggggaggtg
aagaccatcccactagtaacagtctgaacgcattgtttgtgatgcttgagatgctaaattacgtggattataacatcatatt
ccggcgtatgaattgatgagttgtacatcttgacattttcttctttcttctcttctccctttcccagaaacaaactttttttacccac
tataaaataaaatgagtatactacctgttatatttctttctatatttttttattcttcattcgttcagacttttaacgcgcctgaatgt
atcgacaaagggcaatattttgcatcattcatggagttagaaaacgagccagtaatcttaccatgtcctcaaataaata
cgctatcatccggatataatatattagatattttatgggaaaaacgaggagcggataatgatagaattataccgatagat
aatggtagcaatatgctaattctgaacccgacacaatcagactctggtatttatatatgcattaccacgaacgaaacct
actgtgacatgatgtcgttaaatttgacaatcgtgtctgtctcagaatcaaatatagatcttatctcgtatccacaaatagta
aatgagagatctactggcgaaatggtatgtcccaatattaatgcatttattgctagtaacgtaaacgcagatattatatgg
agcgggcatcgacgccttagaaataagagacttaaacaacggacacctggaattattaccatagaagatgttagaa
aaaatgatgctggttattatacatgtgttttagaatatatatacggtggcaaaacatataacgtaaccagaattgtaaaat
tagaggtacgggataaaataataccttctactatgcaattaccagaaggtgttgtaacttcaataggtagtaatttgacta
ttgcgtgtagagtatcgttgagacctcccacaacggatgcagacgtcttttggataagtaatggtatgtattacgaagaa
gatgatggggacggagacggtagaataagtgtagcaaataaaatctatatgaccgataagagacgtgttattacatc
ccggttaaacattaatcctgtcaaggaagaagatgctacaacgtttacgtgtatggcgtttactattcctagcatcagca
aaacagttactgttagtataacgtgaatgtatgttgttacatttccatgtcaattgagtttataagaatttttatacattatcttcc
aacaaacaattgacgaacgtattgctatgattaactcccacgatactatgcatattattaatcattaacttgcagactata
cctagtgctattttgacatactcatgttcttgtgtaattgcggtatctatattattaaagtacgtaaatctagctatagttttattat
ttaattttagataatataccgtctccttatttttaaaaattgccacatcctttattaaatcatgaatgggaatttctatgtcatcgtt
aatatattgtgaacaacaagagcagatatctataggaaagggtggaatgcgatacattgatctatgtagttttaaaaca
cacgcgaactttgaagaatttatataaatcattccatcgatacatccttctatgttgacatgtatatatccaggaattcttttat
taatgtcaggaaatgtataaactaaaacattgcccgaaagcggtgcctctatctgcgttatatccgttcttaacttacaaa
atgtaaccaatacctttgcatgacttgttttgttcggcaacgttagtttaaacttgacgaatggattaattacaatagcatga
tccgcgcatctattaagtttttttactttaacgcccttgtatgtttttacagagactttatctaaatttctagtgcttgtatgtgttata
aatataacgggatatagaactgaatcacctaccttagatacccaattacattttatcagatccagataataaacaaatttt
gtcgccctaactaattctatattgttatatattttacaattggttatgatatcatgtaataacttggagtctaacgcgcatcgtc
gtacgtttatacaattgtgatttagtgtagtatatctacacatgtatttttccgcactatagtattctggactagtgataaaact
atcgttatatctgtcttcaatgaactcatcgagatattgctctctgtcatattcatacacctgcataaactttctagacatctta
caatccgtgttattttaggatcatatttacatatttacgggtatatcaaagatgttagattagttaatgggaatcgtctataata
atgaatattaaacaattatatgaggacttttaccacaaagcatcataaaaatgagtcgtcgtctgatttatgttttaaatatc
aaccgcgaatcaactcataaaatacaagagaatgaaatatatacatattttagtcattgcaatatagaccatacttctac
agaacttgattttgtagttaaaaactatgatctaaacagacgacaacctgtaactgggtatactgcactacactgctattt
gtataataattactttacaaacgatgtactgaagatattattaaatcatggagtggatgtaacgatgaaaaccagtagcg
gacgtatgcctgtttatatattgcttactagatgttgtaatatttcacatgatgtagtgatagatatgatagacaaagataaa
aaccacttatcgcatagagactattccaacctactactagagtatataaaatctcgttacatgttattgaaggaagagga
tatcgatgagaacatagtatccactttattagataagggaatcgatcctaactttaaacaagacggatatacagcgttac
attattattatttgtgtctcgcacacgtttataaaccaggtgagtgtagaaaaccgataacgataaaaaaggccaagcg
aattatttctttgtttatacaacatggagctaatctaaacgcgttagataattgtggtaatacaccattccatttgtatcttagt
attgaaatgtgtaataatattcatatgactaaaatgctgttgacttttaatccgaatttcaaaatatgtaataatcatggatta
acgcctatactatgttatataacttccgactacatacaacacgatattcttgttatgttaatacatcactatgaaacaaatgt
tggagaaatgccgatagatgagcgtcgtataatcgtattcgagtttatcaaaacatattctacacgtccggcagattcga
taacttatttgatgaataggtttaaaaatatagatatttatacccgctatgaaggaaagacattattacacgtagcatgtga
atataataatacacacgtaatagattatcttatacgtatcaacggagatataaatgcgttaaccgacaataacaaacac
gctacacaactcattatagataacaaagaaaattccccatataccattaattgtttactgtatatacttagatatattgtaga
taagaatgtgataagatcgttggtggatcaacttccatctctacctatcttcgatataaaatcatttgagaaattcatatcct
actgtatacttttagatgacacattttacaatagacacgttaggaatcgcaattctaaaacgtatcgatacgcattttcaaa
atacatgtcgtttgataaatacgatggtataataactaaatgtcataaagaaacaatattgctcaaactatccactgttct
agacactacactatatgcagttttaagatgccataattcgaaaaagttaagaagatacctcaacgagttaaaaaaata
taataacgataagtcctttaaaatatattctaatattatgaatgagagataccttaatgtatattataaagatatgtacgtgt
caaaggtatatgataaactatttcctgttttcacagataaaaattgtctactaacattactaccttcagaaattatatacgaa
atattatacatgctgacaattaacgatctttataatatatcgtatccacctaccaaagtatagttgtatttttctcatgcgatgt
gtgtaaaaaaactgatattatataaatattttagtgccgtataatgaagatgacgatgaaaatgatggtacatatatatttc
gtatcattattgttattgctattccacagttacgccatagacatcgaaaatgaaatcacagaattcttcaataaaatgaga
gatactctaccagctaaagactctaaatggttgaatccagcatgtatgttcggaggcacaatgaatgatatagccgctc
taggagagccattcagcgcaaagtgtcctcctattgaagacagtcttttatcgcacagatataaagactatgtggttaaa
tgggagaggctagaaaaaaatagacggcgacaggtttctaataaacgtgttaaacatggtgatttatggatagccaa
ctatacatctaaattcagtaaccgtaggtatttgtgtaccgtaactacaaagaatggtgactgtgttcagggtatagttag
atctcatattaaaaaacctccttcatgcattccaaaaacatatgaactaggtactcatgataagtatggcatagacttata
ctgtggaattctttacgcaaaacattataataatataacttggtataaagataataaggaaattaatatcgacgatattaa
gtattcacaaacgggaaagaaattaattattcataatccagagttagaagatagtggaagatacaactgttacgttcatt
acgacgacgttagaatcaagatgtaaaatacttacggttataccgtcgcaagaccacaggtttaaactaatactagat
ccaaaaatcaacgtaacgataggagaacctgccaatataacatgcactgctgtgtcaacgtcattattgattgacgat
gtactgattgaatgggaaaatccatccggatggcttataggattcgattttgatgtatactctgttttaactagtagaggcg
gtatcaccgaggcgaccttgtactttgaaaatgttactgaagaatatataggtaatacatataaatgtcgtggacacaa
ctattattttgaaaaaacccttacaactacagtagtattggagtaaatacacaatgcatttttatatacattactgaataatt
attattattatttatatcgtatttgtgctataacgcgactatctaggtatttgtatctcaccgatagagaacatataaatgtaga
ctctattaaacagttgtgtaaaatatcagatcctaatagatgtggatgtacggctttagaaatgagttcattaaaatatgtg
atatcaacggaacatatttatataattatactattgctgttagtataattattgattccacggaagaactaccaacagttact
ccaattacaacaacatataattatactatcgatgatagcactactgaagaactacaagtgactcctcatatggatctcca
tcgatgatacatgtattaaaatactttccgaataagtcttttaaatattgtattaattatgaaaaactatgctatgcgagtatg
atacgatactagattttatctctagcgagagatgtcgttagaatcatttatcaacgaatatcgataacatgtgtcatttatac
gttaaagtctgtccgtcttctctattgtttagactgtttgtagaatgctgtgatataaacaaactagtagacacaaatatttaa
ctcatgatgaagttgagaatgatatgctttagctaatataaaaatatattaatccactatatattctagacttgatttaaaac
cgataaactactactacgtactgtataagttaggagcagaccctaattatgtagatgatagaggtaatacttctgcatcta
tatgtccacttatgagaaaacgtcatttaataagatgcatcgtgaaaagaaatttattaaagagttggtaaaatatgaaa
ccgaaagtaaataatataggaaatacacctctacataactacgtatctcaatatgatatcactctcattcctcatccaca
acccattaaaaaatggaaattaaagccctctattagcataaacggctacaggtctacctttacaatggcctttccttgtgc
ccagttcagaccctgtcattgccacgctactaaggactccctgaataccgtggccgacgtcagacattgtctgactgaa
tacatcctgtgggtttctcatagatggacccatagagaaagcgcagggtctctctacaggcttctcatctctttcagaact
gatgcaacggagctctttggtggtgagttgaaggattcacttccgtggagatcattaaatgactccatgaaaaccgccg
aagaacttcgtgcaatcattggactttgtactcaatcagctatcgtctctggaagagtcttcaacgataagtatatcgaca
tactacttatgctgcgaaagattctgaacgagaacgactatctcaccctcttggatcatatccgcactgctaaatactaa
atctccttcatgctctctcactacactttttatcatcttatgaggaataattagcaccagaatagctatggattgcacatgtatt
ctatgtcgtctactggatgaagatgtgacgtacaaaaaaataaaactagaaattgaaacgtgtcacaacttatcaaaa
catatagatagacgaggaaacaatgcgctacattgttacgtctccaataaatgcgatacagacattaagattgttcgac
tgttactctctcgcggagtcgagagactttgtagaaacaacgaaggattaactccgctaggagcatacagtaagcata
gatacgtaaaatctcagattgtgcatctactgatatccagctattcgaattcctctaacgaactcaagtcgaatataaatg
atttcgacttacgtctgctaaaatacctaattgtggataaacggatacgtccgtccaagaatacgaattatgcaatcaat
ggtctcggattggtggatatatacgtaacgacgcctaatccgagaccagaagtattgctatggcttcttaaatcagaatg
ttacagcaccggttacgtatttcgtacctgtatgtacaacagtgatatgtgtaagaactctcttcattactatatatcgtctca
tagagaatctctatccaaggatgtaattaaatgtttgatcgataacaatgtttccatccaatactactggtcttgctcaacc
atagatatagagattattaataaaggatgtggacacgtgtagagtatacgacgtcagccctatattagaggcgtattatc
taaacaagcgatttagagtaaccccatataatgtagacatggaaatcgttaatcttcttattgagagacgtcatactcttgt
cgacgtaatgcgtagtattacttcgtacgattccagagaatataaccactacatcatcgataacattctaaagagattta
gacaacaggatgaatccatcgtacaagccatactgataaactacttacattacggcgatatggtaagtatacctatcat
tcaatgcatgttggataagacgacggacaacaactttgttaataataatctcgtcgatgtaaacgtcgtaaggtttatcgt
ggaaaatatggacacgcggctgtaaatcacatatctaacaatggccgtctatgtatgtacggtctgatattatcgagattt
aataattgcgggtatcactgttatgaagatgtatttgatatactaagcaagtacatggatgatatagatatgatcgataac
tctactatattacgcggtcgatgtcaataatatacaatttgcaaagcggttattggaatatggagcgagtgtcacgctcga
taatcaatacggccatccagaaaagcagttaccaaagagaagctagttgatttattactgagttaccatcccactctag
agactatgattgacgcatttaatagagatatacgctatctatatcctgaaccattattcgcctgtatcagatacgccttaat
cctagatgatgattttccttctaaagtaagtatgatatcgccggtcgtcataaggaactaaagcgctatagagcagacat
taatagaatgaagaatgcctacatatcaggcgtctccatgtttgatatattatttaaacgaagcaaacgccacagattga
gatacgcaaagaacaatgagaggatcgactccattaaataatttatcatggagtgataatgtcctgtttccatggcatat
tacaaaatcgattccgtccaagatgataaaaacatttaccggcatcataaacacggagtttattttatatgtctcgcataa
acattactaaaaaaatatattgttcggttttctttcacatctttaattatgaaaaagtaaatcattatgagatggacgcatcgt
tcgcgacagtatgtggtacatacctaacgtatttatggacgacggtaagaatgaaggtcacgtttctgtcaacaatgtcg
acgcgatcgtgtaacacgactcacaatagaatctgtgaatgctctcccgatcatggatgcaaggcatgtgtttcccaaa
caaaatgtggaataggatacggagtatccggagacgtcatctgttctccgtgtggtctcggaacatattctcacaccgtc
tcttccgcagataaatgcgaacccgtacccagaaatacctttaactatatcgatgtggaaattaacctgtatccagttaa
cgacacatcgtgtactcggacgaccactaccggtctcagcgaatccatctcaacgtcggaactaactattactatgaat
cataaagactgcgatcccgtcttcttaataaggtagcgacttcaggtttctttacaggagaaaggtgtgcactctgaattt
cgagattaaatgcaataacaaagattcttcctccaaacagttaacgaaagcaaagaatgatactatcatgccgcattc
ggagacagtaactctagcgtcgacatctatatactatatagtaataccaatactcaagactacgaaactgatacaatct
cttatcatgtgggtaatgtagccatatgcccggtagttgcgatatacataaactgatcactaattccaaacccacccgctt
tttatagtaagtttttcacccataaatacaataattaatttctcgtaaaagtagaaaatatattctaatttattgcacggtaag
gaagtagaatcataaagaacagtactcaatcaatagcaatcatgaaacaatatatcgtactggcatgcatgtgcctgc
cagtcttcagcaatcatcctcatcgtgtacggaagaagaaaacaaacatcatatgggaatcgatgttattatcaaagtc
acaaagcaagaccaaacaccgaccgatgataagatttgccaatccgtaacggaaattacagagtccgagtcagat
ccagatcccgaggtggaatcagtcgaggatgtagatcctcctaccacttattactccatcatcggtggaggtctgagaa
tgaactttggattcaccaaatgtcctcagattaaatccatctcagaatccgctgatggaaagactgtgaggtgtctatcg
acatcagatgtagcgaagaagagaaagacagcgacatcaagacccatccagtactcgggtctaacatctctcataa
gaaagtgagttacgaagatatcatcggttcaacgatcgtcgatacaaaatgtgtcaagaatctagagtttagcgttcgt
atcggagacatgtgcaaggaatcatctgaacttgaggtcaagtatgtcgacggatcggcatctgaaggtgcaaccga
tgatacttcactcatcgattcaacaaaactcaaagcgtgtgtctgaatcgataactctattcatctgaaattggatgagta
gggttaatcgaacgattcaggcacaccacgaattaaaaaagtgtaccggacactatattccggtttgcaaaacaaaa
agttacctctcgcgacttcttctttttctgtctcaatagtgtgatacgattatgacactattcctatttcctttcagggtatcacaa
aaatattaaacctctttctgatggtctcatacaaaaatatttttattctctttctctctttgatggtctcataaaaaatatttttattct
ctttctctctttgatggtctcataaaatatttttattctctttctctctttgatggtctcataaaaaatatttttattctctttctctctttga
tggtctcataaaatatttttattctctttctctctttgatggtctcataaaaaatatttttattctctttctctctttgatggtctcataaa
aaatattaaacctctttctgatggtgtcactaaaatatttttattctctttctctcttcaatggagtcataaaatatttttattctcttt
ctctcttcgatggtctcacaaaaatattaaacctctttctgatggtgtcactaaaatatttttattctctttctctcttcaatggagt
cataaaatatttttattctctttctctctttgatggtctcacaaaaatatttttattctctttctctctttgatggtctcacaaaaatatt
tttattctctttctctctttgatggtctcacaaaaatatttttattctctttctctctttgatggtctcataaaaaaagttttacaaaaa
tatttttattctctttctctctttgatggtctcataaaaaaagttttacaaaaatatttttattctctttctctctttgatggtctcataaa
aaaagttttacaaaaatatttttattctctttctctctttgatggtctcataaaaaatattaaacctctttctgatggtgtcactaa
aatatttttattctcattctctcttcaatggagtcataaaatatttttattctctttctctcttcgatggtctcacaaaaatattaaac
ctctttctgatggtgtcactaaaatatttttattctcattctctcttcaatggagtcataaaatatttttattctctttctctcttcgatg
gtctcacaaaaatattaaacctctttctgatggtgtcactaaaatatttttattctcattctctcttcaatggagtcataaaatat
ttttattctctttctctctttgatggtctcataaaaaaagttttacaaaaatatttttattctctttctctctttgatggtctcataaaaa
atattaaacctctttctgatggtgtcactaaaatatttttattctctttctctcttcaatggagtcataaaatatttttattctctttctc
tcttcgatggtctcacaaaaatattaaacctctttctgatggtgtcactaaaatatttttattctcattctctcttcaatggagtc
ataaaatatttttattctctttctctctttgatggtctcataaaaaaagttttacaaaaatatttttattctctttctctctttgatggtct
cataaaaaaagttttacaaaaatatttttattctctttctctctttgatggtctcataaaaaaagttttacaaaaatatttttattct
ctttctctctttgatggtctcataaaaaaagttttacaaaaatatttttattctctttctctctttgatggtctcataaaaaatattaa
acctctttctgatggtgtcactaaaatatttttattctcattctctcttcaatggagtcataaaatatttttattctctttctctcttcga
tggtctcacaaaaatattaaacctctttctgatggtgtcactaaaatatttttattctcattctctcttcaatggagtcataaaat
atttttattctctttctctctttgatggtctcataaaaaaagttttacaaaaatatttttattctctttctctctttgatggtctcataaa
aaaagttttacaaaaatatttttattctctttctctctttgatggtctcataaaaaaagttttacaaaaatatttttattctctttctct
ctttgatggtctcataaaaaaagttttacaaaaatatttttattctctttctctctttgatggtctcacaaaaatattaaacctcttt
ctgatggagtcgtaaaaaagttttatctctttctccttcgatggtctcacaaaaatattaaacctctttctgatggagtcgtaa
aaaagttttatctctttctctcttcgatggtctcacaaaaatattaaacctctttctgatggagtcgtaaaaaagttttatctcttt
ctctcttcgatggtctcactaaaatattttttattctctttctgatgcatcaactatttcttaaacaataacgtccaacaacatat
actcgtcgagcttatcaacatcccctatgcccatctaggttaccagacaattgtatatcataaaataatgtttataatttttac
aaaaatatttttattctctttctctctttgatggtctcataaaaaaagttttacaaaaatatttttattctctttctctctttgatggtct
cataaaaaatattaaacctctttctgatggtgtcactaaaatatttttattctcattctctcttcaatggagtcataaaatattttt
attctctttctctcttcgatggtctcacaaaaatattaaacctctttctgatggtgtcactaaaatatttttattctcattctctcttc
aatggagtcataaaatatttttattctctttctctctttgatggtctcataaaaaaagttttacaaaaatatttttattctctttctctc
tttgatggtctcataaaaaaagttttacaaaaatatttttattctctttctctctttgatggtctcataaaaaaagttttacaaaa
atatttttattctctttctctctttgatggtctcataaaaaaagttttacaaaaatatttttattctctttctctctttgatggtctcataa
aaaaagttttacaaaaatatttttattctctttctctctttgatggtctcataaaaaaagttttacaaaaatatttttattctctttct
ctctttgatggtctcataaaaaaagttttacaaaaatatttttattctctttctctctttgatggtctcataaaaaatattaaacct
ctttctgatggtgtcactaaaatatttttattctctttctctcttcaatggagtcataaaatatttttattctctttctctcttcgatggt
ctcacaaaaatattaaacctctttctgatggtgtcactaaaatatttttattctcattctctcttcaatggagtcataaaatatttt
tattctctttctctctttgatggtctcataaaaaaagttttacaaaaatatttttattctctttctctctttgatggtctcataaaaaa
agttttacaaaaatatttttattctctttctctctttgatggtctcataaaaaaagttttacaaaaatatttttattctctttctctctttg
atggtctcataaaaaaagttttacaaaaatatttttattctctttctctctttgatggtctcataaaaaaagttttacaaaaatat
ttttattctctttctctctttgatggtctcataaaaaatattaaacctctttctgatggtgtcactaaaatatttttattctctttctctct
tcaatggagtcataaaatatttttattctctttctctcttcgatggtctcacaaaaatattaaacctctttctgatggtgtcacta
aaatatttttattctctttctctcttcaatggagtcataaaatatttttattctctttctctctttgatggtctcataaaaaaagttttac
aaaaatatttttattctctttctctctttgatggtctcataaaaaaagttttacaaaaatatttttattctctttctctctttgatggtct
cataaaaaaagttttacaaaaatatttttattctctttctctctttgatggtctcataaaaaaagttttacaaaaatatttttattct
ctttctctctttgatggtctcataaaaaaagttttacaaaaatatttttattctctttctctctttgatggtctcataaaaaaagtttt
acaaaaatatttttattctctttctctctttgatggtctcataaaaaaagttttacaaaaatatttttattctctttctctctttgatggt
ctcataaaaaatattaaacctctttctgatggtgtcactaaaatatttttattctcattttctctttctctcttcaatggagtcataa
aatatttttattctctttctctctttgatggtctcataaaaaatattaaacctctttctgatggtgtcactaaaatatttttattctcatt
ctctcttcaatggagtcataaaaaagttttatctctttctctcttcgatggtctcacaaaaatattaaacctctttctgatggag
tcgtaaaaaagttttatctctttctctcttcgatggtctcacaaaaatattaaacctctttctgatgcatcaactatttcttaaac
aataacgtccaacaacatatactcatcccctatgcccatctaggttaccagacaattgtatatcataaaataatgtttata
atttacacgttaaaatcatataataaaacgtagatcgtataatattttttggtatataaatgatctagtaaaatccatgtagg
ggatactgctcacattttttctttggtacaaaatttcacacaagtttttatacagacaaattcttgtccatatattttaaaacatt
gacttttgtactaagaaaaatatctagactaactatctctttctctttctctcttcgatggtctttctgatggagtcgtaaaaaa
gttttatctctttctctcttcgatggtctcacaaaaatattaaacctctttctgatggagtcgtaaaaaagttttatctctttctctct
tcgatggtctcacaaaaatattaaacctctttctgatggagtcgtaaaaaagttttatctctttctccttcgatggtctcacaa
aaatattaaacctctttctgatggagtcgtaaaaaagttttatctctttctctcttcgatggtctcacaaaaatattaaacctct
ttctgatggtgtcactaaaatatttttattctctttctctcttcgatggtctcacaaaaatattaaacctctttctgatggagtcgt
aaaaaagttttatctctttctccttcgatggtctcacaaaaatattaaacctctttctgatggagtcgtaaaaaagttttatctc
tttctctcttcgatggtctcacaaaaatattaaacctctttctgatggagtcgtaaaaaagttttatctctttctccttcgatggtc
tcacaaaaatattaaacctctttctgatggagtcgtaaaaaagttttatctctttctccttcgatggtctcacaaaaatattaa
acctctttctgatggagtcgtaaaaaagttttatctctttctccttcgatggtctcacaaaaatattaaacctctttctgatgga
gtcgtaaaaaagttttatctctttctctcttcgatggtctcacaaaaatattaaacctctttctgatggagtcgtaaaaaagttt
tatctctttctctcttcgatggtctcacaaaaatattaaacctctttctgatggagtcgtaaaaaagttttatctctttctccttcg
atggtctcacaaaaatattaaacctctttctgatggtctctataaagcgattgatttttcttaccctctagagtttcctacggtc
gttggtcacacatttttttctagacactaaataaatagtaagattaaattaattataaaattatgtatataatattaattataaa
attatgtatatgatttactaactttagttagataaattaataatacataaattttagtatattaatattataaattaataatacata
aattttagtatattaatattatattttaaatatttatttagtgtctagaaaaaaaggccggcctagcaggcc

Alternatively, the sequence of sMVA F3 may contain 1 nucleotide alteration in a non-coding determining region at 88 bp close to the end of the ITR sequence (deposited with NCBI under Accession No. MW030459, www.ncbi.nlm.nih.gov/nuccore/MW030459.1/) (SEQ ID NO: 15):

ggcctagcaggcctttttttctagacactaaataaatagtaagattaaattaattataaaattatgtatataatattaattata
aaattatgtatatgatttactaactttagttagataaattaataatacataaattttagtatattaatattataaattaataat
acataaattttagtatattaatattatattttaaatatttatttagtgtctagaaaaaaaggccggccgggtattaataatatc
tatatttccagcgttaagtgtaacattaaacagttttaattcacgtgacgtggtatcaattaaataattaatgcccaatttgga
tatagcagcctgaagctcatcttgtttagttacggatcctaatgagttattaagcaatatatcgaacggatgaacgaaggttgt
tttaagttggtcacatactttgtaatctagacatagatgcggaagaacggtagaaactatacgaaataaatattcagagtcctc
taattgatcaagagtaactattgacttaataggcatcatttatttagtattaaatgacgaccgtaccagtgacggatatacaaa
acgatttaattacagagttttcagaagataattatccatctaacaaaaattatgaaataactcttcgtcaaatgtctattctaa
ctcacgttaacaacgtggtagatagagaacataatgccgccgtagtgtcatctccagaggaaatatcctcacaacttaatgaag
atctatttccagatgatgattctccggccactattatcgaacgagtacaacctcatactactattattgacgatactccacctc
ctacgtttcgtagagagttattgatatcggaacaacgtcaacaacgagaaaaaagatttaatattacagtatcgaaaaatgctg
aagcaataatggaatctagatctatgatatcttctatgccaacacaaacaccatccttgggagtagtttatgataaagataaaa
gaattcagatgttggaggatgaagtggttaatcttagaaatcaacgatctaatacaaaatcatctgataatttagataatttta
ccagaatactatttggtaagactccgtataaatcaacagaagttaataagcgtatagccatcgttaattatgcaaatttgaacg
ggtctcccttatcagtcgaggacttggatgtttgttcagaggatgaaatagatagaatctataaaacgattaaacaatatcacg
aaagtagaaaacgaaaaattatcgtcactaacgtgattattattgtcataaatattatcgagcaagcattgctaaaactcggat
ttgaagaaatcaaaggactgagtaccgatatcacttcagaaattatcgatgtggagatcggagatgactgcgatgctgtagcat
caaaactaggaatcggtaacagtccggttcttaatattgtattgtttatactcaagatattcgttaaacgaattaaaattattt
aatttaatacattcccatatccagacaacaatcgtctggattaatctgttcctgtcgtctcataccggacgacatattaatctt
tttattagtaggcatctttttagatggtttctttttcccagcattaactgagtcgatacctagaagatcgtgattgatctctcc
gaccattccacgaacttctaattggccgtctctgacggtaccataaactattttaccagcattagtaacagcttggacaatctg
accatccatcgcattgtacgatgtagtagtaactgttgttctacgtctaggagcaccagaagtatttttggagcccttggatgt
tgatgtagaagaagacgaggattttgattttggtttacatgtaatacattttgtatcacatgcgccggcagtcacatctgtttg
agaattaagattattgttgcctcctttgacggctgcatctccaccgatttgcgctagtagatttttaagctgtggtgtaatctt
attaactgtttcgatataatcatcgtaactgcttctaacggctaaattttttttatccgccatttagaagctaaaaatattttt
atttatgcagaagatttaactagattatacaatgaactaatatgatccttttccagattatttacaaacttggtattttttggt
tctggaggaggcgaatttaaattcggacttggatttggattttgtgggttcttgatcttattatacagcgcatataggatggcg
acggtaactgctacgcaaataccgatcaacaaaagaataccaatcatttattgacaataacttcactattgatcaagtatgcaa
tatatcatcttttcactaaataagtagtaataatgattcaacaatgtcgagatatatggacgataataatttagttcatggaaa
tatcgctatgattggtatgaatgactccgctaactctgtggggcgcgcagtgctttccccacatagaataaattagcattccga
ctgtgataataataccaagtataaacgccataatactcaatactttccatgtacgagtgggactggtagacttactaaagtcaa
taaaggcgaagatacacgaaagaatcaaaagaatgattccagcgattagcacgccggaaaaataatttccaatcataagcatca
tatccatttaactaataaaaattttaaatcgccgaatgaacaaagtggaatataaaccatataaaaacaatagtttgtactgca
aaaataatatctatttttgttttcgaagatatggtaaaattaaatagtagtacacagcatgttataactaacagcagcaacggc
tcgtaattacttatcatttactagacgaaaaggtggtgggatattttcttgctcaaataatacgaatatatcacccatccattt
tatgcgatgtttatatactctaatctttaatagatctatagacgacgggtttaccaacaatatagattttatcgattcatctaa
tttaaacccttccttaaacgtgaatgatctattatctggcataacgatgactctacccgatgaatcggacaatgtactgggcca
tgtagaataaattatcaacgaattatcgtctacgaacatttatatcatttgttttaattttagtacgcgaataaatagatataa
aatagaaaataacagatattacaaccaatgttatggccgcgcccaaccaggtaggcagttttattttatcttttactacaggtt
ctcctggatgtacgtcaccaacggcggacgtagttctagtacaattagacgtaagttccgcttgggaattttttaacgctaaag
agttaacgttaatcgtgcacccaacgtatttacatctagttctttgaacatcttgattataatataaccattttctatctctag
attcgtcggtgcactcatgtaaccaacataccctaggtcctaaatatttatctccggaattagattttggataattcgcgcacc
aacaatttctatttcctttatgatcgttacaaaagacgtataatgccgtatccccaaaagtaaaataatcaggacgaataattc
taataaactcagaacaatatctcgcatccatatgtttggagcaaatatcggaataagtagacatagccggtttccgttttgcac
gtaaccattctaaacaattggggtttccaggatcgtttctacaaaatccagtcatgaaatcatcacaatgttctgtcttgtaat
tattattaaatatttttggacagtgtttggtatttgtcttagaacaacattttgccacgctatcactatcgcccaggagataat
ccttttttataaaatgacatcgttgcccggatgctatataatcagtagcgtgttttaaatccttaatatattcaggagttacct
cgttctgataatagattaatgatccaggacgaaatttgaaagaactacatggttctccatgaattaatacatattgtttagcaa
attcaggaactataaaactactacaatgatctatcgacataccatctatcaaacaaaacttgggtttaatttctcccggagatg
tttcataatagtacgtataactttcttctgcaaacttaacagctctattatattcaggataattaaaacctaattccatatatt
tgtctcgtatatctgctattcctggtgctattttgattctattaagagtaacggctgcccccatttttaataatcgtcagtatt
taaactgttaaatgttggtatatcaacatttaccttatttcccgcagtataaggtttgttgcaggtatactgttcaggaatggt
tacatttatactttttctatagtcctgtctttcgatgttcatcacatatgcaaagaacagaataaacaaaataatgtaagaaat
aatattaaatatctgtgaattcgtaaatacattgattgccataataattacagcagctacaatacatacaatagacattcccac
agtgttgccattacctccacgatacatttgagttactaagcaataggtaataactaagctagtaagaggcaatagaaaagatga
gataaatatcatcaatatagagattagaggagggctatatagagccaagacgaacaaaatcaaaccgagtaacgttctaacatc
attatttttgaagattcccaaataatcattcattcctccataatcgttttgcatcatacctccatctttaggcataaacgattg
ctgctgttcctctgtaaataaatctttatcaagcactccagcacccgcagagaagtcgtcaagcatattgtaatatcttaaata
actcatttatatattaaaaaatgtcactattaaagatggagtataatctttatgccgaactaaaaaaaatgacttgtggtcaac
ccctaagtctttttaacgaagacggggatttcgtagaagttgaaccgggatcatcctttaagtttctgatacctaagggatttt
acgcctctccttccgtaaagacgagtctagtatttgaaacattaacaacgaccgataataaaattactagtatcaatccaacaa
atgcgccaaagttatatcctcttcaacgcaaagtcgtatctgaagtagtttctaatatgaggaaaatgatcgaatcaaaacgtc
ctctatacatcactcttcacttggcgtgtggatttggtaagactattaccacgtgttatcttatggctacacacggtagaaaaa
ccgtcatttgcgtacccaataaaatgttaatacatcaatggaagacacaggtagaggcagtcggattggaacataagatatcca
tagatggagtaagtagtctattaaaggaactaaagactcaaagtccggatgtattaatagtagtcagtagacatctgacaaacg
atgccttttgtaaatatatcaataagcattatgatttgttcatcttggatgaatcacatacgtataatctgatgaacaatacag
cagttacaagatttttagcgtattatcctccgatgatgtgttattttttaactgctacacctagaccatctaacagaatttatt
gtaacagtattattaatattgccaagttatccgatctaaaaaaaactatctatgcagtagatagtttttttgagccatattcca
cagataatattagacatatgataaaacgattagatggaccatctaataaatatcatatatataccgagaagttattatctgtag
acgagcctagaaatcaacttattcttaataccctggtagaagaattcaagtcaggaactattaatcgcattttagttattacta
aactacgtgaacatatggtattcttctacaaacgattattagattttttcggatcagaggttgtatttataggagacgcccaaa
atagacgtactccagatatggtcaaatcaatcaaggaactaaatagatttatattcgtatccaccttattttattccggtactg
gtttagatattcctagtttggattctttgttcatttgctcggcagtaatcaacaatatgcaaatagagcaattactagggaggg
tatgtcgagaaacagaactattagataggacggtatatgtatttcctagcacatccatcaaagaaataaagtacatgataggaa
atttcgttcaacgaattattagtctgtctgtagataaactaggatttaaacaaaaaagttatcggaaacatcaagaatccgatc
ccacttctgtatgtacaacatcctccagagaagaacgtgtattaaatagaatatttaactcgcaaaatcgttaagaagtttaag
cgacgatccgcatgctgcgcaggccagtgtattacccctcatagtattaatataatccaatgatacttttgtgatgtcggaaat
cttaaccaatttagactgacaggcagaacacgtcatgcaatcatcatcgtcatcgataactgtagtcttgggcttctttttgcg
gctcttcattccggaacgcacattggtgctatccatttaggtagtaaaaaataagtcagaatatgccctatagcacgatcgtgc
aaaacctggtatatcgtctctatctttatcacaatatagtgtatcgacatctttattattattgacctcgtttatcttggaaca
tggaatgggaacatttttgttatcaacggccacctttgccttaattccagatgttgtaaaattataactaaacagtctatcatc
gacacaaatgaaattcttgtttagacgtttgtagtttacgtatgcggctcgttcgcgtctcattttttcagatattgcaggtac
tataatattaaaaataagaatgaaataacataggattaaaaataaagttatcatgacttctagcgctgatttaactaacttaaa
agaattacttagtctgtacaaaagtttgaaattttcagattctgcggctatagaaaagtataattctttggtagaatggggaac
atctacttactggaaaataggcgtgcaaaaggtagctaatgtcgagacgtcaatatctgattattatgatgaggtaaaaaataa
accgtttaatattgatccgggctattacattttcttaccggtatattttgggagcgtctttatttattcgaagggtaaaaatat
ggtagaacttggatctggaaactcttttcaaataccagatgatatgcgaagtgcgtgtaacaaagtattagacagcgataacgg
aatagactttctgagatttgttttgttaaacaatagatggataatggaagatgctatatcaaaatatcagtctccagttaatat
atttaaactagctagtgagtacggattaaacatacccaaatatttagaaattgaaatagaggaagacacattatttgacgacga
gttatactctattatagaacgctctttcgatgataaatttccaaaaatatccatatcgtatattaagttgggagaacttaggcg
gcaagttgtagactttttcaaattctcattcatgtatattgagtccatcaaggtagatcgtataggagataatatttttattcc
tagcgttataacaaaatcaggaaaaaagatattagtaaaagatgtagaccatttaatacgatccaaggttagagaacatacatt
tgtaaaagtaaaaaagaaaaacacattttccattttatacgactatgatggaaacggaacagaaactagaggagaagtaataaa
acgaattatagacactataggacgagactattatgttaacggaaagtatttctctaaggttggtagtgcaggcttaaagcaatt
gactaataaattagatattaatgagtgcgcaactgtcgatgagttagttgatgagattaataaatccggaactgtaaaacgaaa
aataaaaaaccaatcagcatttgatttaagcagagaatgtttgggatatccagaagcggattttataacgttagttaataacat
gcggttcaaaatagaaaattgtaaggttgtaaatttcaatattgaaaatactaattgtttaaataacccgagtattgagactat
atatggaaactttaaccagttcgtctcaatctttaatatcgtcaccgatgtcaaaaaaagattattcgagtgaaataatatgcg
cctttgatataggtgcaaaaaatcctgccagaactgttttagaagtcaaggataactccgttagggtattggatatatcaaaat
tagactggagttctgattgggaaaggcacatagctaaagatttgtcacaatatgaatacactacagttcttctagaacgtcagc
ctagaaggtcgccgtacgtcaaatttatctattttattaaaggctttttatatcatacatcggctgccaaagttatttgcgtct
cgcctgtcatgtctggtaattcatatagagatcgaaaaaagagatcggtcgaagcatttcttgattggatggacacattcggat
tgcgagactccgttccggatagacgcaaattagacgatgtagcggatagtttcaatttggctatgagatacgtattagataaat
ggaatactaattatacaccttataataggtgtaaatctagaaattacataaaaaaaatgtaataacgttagtaacgccattatg
gataatctatttacctttctacatgaaatagaagatagatatgccagaactatttttaactttcatctaataagttgcgatgaa
ataggagatatatatggtcttatgaaagaacgcatttcctcagaggatatgtttgataatatagtgtataataaagatatacat
catgccattaagaaactagtgtattgcgacatccaacttactaaacacattattaatcagaatacgtatccggtatttaacgat
tcttcacaagtgaaatgttgtcattatttcgatataaactcagataatagcaatattagctctcgtacagtagagatatttgag
agggaaaagtcatctcttgtatcatatattaaaactaccaataagaagagaaaggtcaattatggggaaataaagaaaactgta
catggaggcactaatgcaaattacttttccggtaaaaagtctgatgagtatctgagcactacagtcaggtccaacattaatcaa
ccttggatcaaaaccatttctaagagaatgagagtagatatcattaatcactctatagtaacgcgtggaaaaagctctatatta
caaactatagaaattatttttactaatagaacatgtgtgaaaatattcaaggattctactatgcacattattctatccaaggac
aaggatgaaaaggggtgtatacacatgattgacaaattattctatgtctattataatttatttctgttgttcgaggatatcatc
caaaacgagtactttaaagaagtagctaatgttgtaaaccacgtactcacggctacggcattagatgagaaattattcctaatt
aagaaaatggctgaacacgatgtttatggagttagcaatttcaaaatagggatgtttaacctgacatttattaagtcgttggat
cataccgttttcccctctctgttagatgaggatagcaaaataaagttttttaaggggaaaaagctcaatattgtagcattacga
tctctggaggattgtataaattacgtgactaaatccgagaatatgatagaaatgatgaaggaaagatcgactattttaaatagc
atagatatagaaacggaatcggtagatcgtctaaaagaattgcttctaaaatgaaaaaaaaacactaattcagaaatggatcaa
cgactcggatataagtttttggtgcctgatcctaaagccggagttttttatagaccgttacatttccaatatgtatcgtattct
aattttatattgcatcgattgcatgaaatcttgaccgtcaagcggccactcttatcgtttaagaataatacagaacgaattatg
atagaaattagcaatgttaaagtgactcctccagattactcacctataatcgcgagtattaaaggtaagagttatgacgcatta
gccacgttcactgtaaatatctttaaagaggtaatgaccaaagagggtatatccatcactaaaataagtagttatgagggaaaa
gattctcatttgataaaaattccgctactaataggatacgggaataaaaatccacttgatacagccaagtatcttgttcctaat
gtcataggtggagtctttatcaataaacaatctgtcgaaaaagtaggaattaatctagtagaaaagattacaacatggccaaaa
tttagggttgttaagccaaactcattcactttctcgttttcctccgtatcccctcctaatgtattaccgacaagatatcgccat
tacaagatatctctggatatatcacaattggaagcgttgaatatatcatcgacaaagacatttataacggtcaatattgttttg
ctgtctcaatatttatctagagtgagtctagaattcattagacgtagtttatcatacgatatgcctccagaagttgtctatcta
gtaaacgcgataatagatagtgctaaacgaattactgaatctattactgactttaatattgatacatacattaatgacctggtg
gaagctgaacacattaaacaaaaatctcagttaacgatcaacgagttcaaatatgaaatgctgcataactttttacctcatatg
aactatacacccgatcaactaaagggattttatatgatatctttactaagaaagtttctctactgtatctaccacacttctaga
tatccagatagagattcgatggtttgtcatcgcatcctaacgtacggcaaatattttgagacgttggcacatgatgaattagag
aattacataggcaacatccgaaacgatatcatgaacaatcacaagaacagaggcacttacgcggtaaacattcatgtactaaca
actcctggacttaatcatgcattttctagtctattgagtggaaagttcaaaaagtcagacggtagttatcgaacacatcctcac
tattcatggatgcagaatatttctattcctaggagtgttggattttatccggatcaagtaaagatttcaaagatgttttctgtc
agaaaataccatccaagtcaatatctttacttttgttcatcggacgttccggaaagaggtcctcaggtaggtttagtatctcaa
ttgtctgtcttgagttccattacaaatatactaacgtctgagtatttggatttggaaaagaaaatttgtgagtatatcagatca
tattataaagatgatataagttactttgaaacaggatttccaatcactatagaaaatgctctagtcgcatctcttaatccaaat
atgatatgtgattttgtaactgactttagacgtagaaaacggatgggattcttcggtaacttggaggtaggtattactttagtt
agggatcacatgaatgaaattcgcattaatattggagcgggaagattagtcagaccattcttggttgtggataacggagagctc
atgatggatgtgtgtccggagttagaaagcagattagacgacatgacattctctgacattcagaaagagtttccgcatgtcatc
gaaatggtagatatagaacaatttacttttagtaacgtatgtgaatcggttcaaaaatttagaatgatgtcaaaggatgaaaga
aagcaatacgatttatgtgactttcctgccgaatttagagatggatatgtagcatcttcactagtgggaatcaatcacaattct
ggacccagagctattcttggatgtgctcaagctaaacaagctatctcttgtctgagctcggatatacgaaataaaatagacaat
ggaattcatttgatgtatccagagaggccaatcgtgattagtaaggctttagaaacttcaaagattgcggctaattgcttcggc
caacatgttactatagcattaatgtcgtacaaaggtatcaatcaagaggatggaattatcatcaaaaaacaatttattcagaga
ggcggtctcgatattgttacagccaagaaacatcaagtagaaattccgttggaaaactttaataacaaagaaagagataggtct
aacgcctattcaaaattagaaagtaatggattagttagactgaatgctttcttggaatccggagacgctatagcacgaaatatc
tcatcaagaactcttgaagatgattttgctagagataatcagattagctttgatgtttccgaaaaatataccgatatgtacaaa
tctcgcgttgaacgagtacaagtagaacttactgacaaagttaaggtacgagtattaaccatgaaagaaagaagacccattcta
ggagacaaatttaccactagaacgagtcaaaagggaacagtcgcgtatatcgcggatgaaacggaacttccatacgatgaaaat
ggtatcacaccagatgtcattattaattctacatccatcttctctagaaaaactatatctatgttgatagaggttattttaaca
gccgcatattctgctaagccgtacaacaataagggagaaaaccgacctgtctgttttcctagtagtaacgaaacatccatcgat
acatatatgcaattcgctaaacaatgttatgagcattcaaatccgaaattgtctgatgaagaattatcggataaaatcttttgt
gaaaagattctctatgatcctgaaacggataagccttatgcatccaaagtattttttggaccaatttattacttgcgtctgagg
catttaactcaggacaaggcaaccgttagatgtagaggtaaaaagacgaagctcattagacaggcgaatgagggacgaaaacgt
ggaggaggtatcaagttcggagaaatggagagagactgtttaatagcgcatggtgcagccaatactattacagaagttttgaaa
gattcggaagaagattatcaagatgtgtatgtttgtgaaaattgtggagacatagcagcacaaatcaagggtattaatacatgt
cttagatgttcaaaacttaatctctctcctctcttaacaaaaattgataccacgcacgtatctaaagtatttcttactcaaatg
aacgccagaggcgtaaaagttaaattagatttcgaacgaaggcctccttcgttttataaaccattagataaagttgatctcaaa
ccgtcttttctggtgtaatattctagtttggtagtagatacatatcaatatcatcaaattcgagatccgaattataaaatgggc
gtggattgttaactatagaatcggacgtctgatattcgaaaatctgtggagttttaggttttggtggaggtgtaactgctactt
gggatactgaagtctgatattcagaaagctgggggatgttctggttcgacatccaccgatggtgtcacatcactaatcggttcg
gtaacgtctgtggacgatggaggcaccacttctacaggttctggttctttatcctcagtcatcaacggagctacttcaatgcga
ggaaatgtataatttggtaatggtttctcatgtggatctgaagaagaggtaagatatctactagaaagataccgatcacgttct
agttctcttttgtagaacttaactttttctttctccgcatctagttgatattccaacctcttcacgttcgcatgggttacctcc
gcagtttttacgagcgatttcacgttcagccttcatgcgtcttatagcatgaattcgcttatcgttatcgggtttagcttctgt
caccttagcaattccttttttattaaactctacataatcatatccatttctattgtttgttctaatataaacgagtatagcatc
attgctaaatttttcaatagtatcgaaaacagaatatcctaaaccatataatatatattcaggaacactcaaactaaatgtcca
ggattctcctaaatacgtaaactttaatagtgcgaaatcattcaaaaatctaccacttatagatagatagatagtacataaatg
cgtatagtagtctacctatctctttattatgaaaaccggcattacgatcatatatgtcgtgatatacctgtgatccgtttacgt
taaaccataaatacatgggtgatcctataaacatgaatttatttctaattctcagagctatagttaattgaccgtgtaatattt
gcttacatgcatacttgatacgatcattaataagatttttatcattgctcgttatttcagaatcgtatatataaggagtaccat
cgtgattcttaccagatattatacaaaatactatatataaaatatattgacccacgttagtaatcatgtaaatgtttaacgttt
taaattttgtattcaatgatccattatcatacgctagcatggtcttatgatattcattctttaaaatataatattgtgttagcc
attgcattggggctcctaatggagattttttattctcatccattttaggataggctttcataaagtccctaataacttcgtgaa
taatgtttctatgttttctactgatgcatgtatttgcttcgatttttttatcccatgtttcatctatcatagatttaaacgcag
taatgctcgcaacattaacatcttgaaccgttggtacaattccgttccataaatttataatgttcgccatttatataactcatt
ttttgaatatacttttaattaacaaaagagttaagttactcatatggacgccgtccagtctgaacatcaatctttttagccaga
gatatcatagccgctcttagagtttcagcgtgattttccaacctaaatagaacttcatcgttgcgtttacaacacttttctatt
tgttcaaactttgttgttacattagtaatctttttttccaaattagttagccgttgtttgagagtttcctcattgtcgtcttca
tcggctttaacaattgcttcgcgtttagcctctggctttttagcagccgttgtagaaaaaaattcagttgctggaattgcaaga
tcgtcatctccggggaaaagagttccgtccatttaaagtacagattttagaaactgacactctgcgttatttatatttggtaca
acacatggattataaatattgatgttaataacatcagaaaatgtaaagtctatacattgttgcatcgtgttaaattttctaatg
gatctagtattattgggtccaacttctgcctgaaatccaaatatggaagcggatacaaaaccgtttcctggataaaccacacat
ctccacttttgctttacatcagaaattgtgtcgttgacatcttgaactctcctatctaatgccggtgttccacctatagatttt
gaatattcgaatgctgcatgagtagcattaaattccttaatattgccataattttcatatattgagtaaccctggataaaaagt
aaacacaccgcagccgtagctaccacaataaaaaaaattgatagagagttcatttataatctattagaagctgacaaaattttt
ttacacgcatcagacaatgctttaataaatagttcaacatctacttttgtcatatcgaaccgatggtatgattctaacctagaa
ttacatccgaaaaagttgactatgttcatagtcattaagtcattaacaaacaacattccagactctggattataagacgatact
gtttcgtcacaattacctaccttaatcatgtgattatgaatattggctattagagcaccttctaagaaatctataatatctttg
aaacacgatttaaaatcaaaccacgaatatacttctacgaagaaagttagtttacccataggagaaataactataaatggagat
ctaaatacaaaatccggatctatgatagttttaacattattatattctctattaaatacctccacatctaaaaatgttaatttt
gaaactatgtcttcgtttattaccgtacctgaactaaacgctataagctctattgtttgagaactctttaaacgatattcttga
aatacatgtaacaaagtttcctttaactcggtcggtttatctaccatagttacagaatttgtatccttatctataatataataa
tcaaaatcgtataaagttatataattatcgcgttcagattgggatcttttcaaatagactaaaaaccccatttctctagtaagt
atcttatgtatatgtttgtaaaatatcttcatggtgggaatatgctctaccgcagttagccattcctcattgacagcggtagat
gtattagacaaaactattccaatgtttaacaagggccattttacgagattattaaatccttgtttgataaatgtagccaatgag
ggttcgagttcaacgacgattgaattctcttcccgcggatgctgcatgatgaacgacgggatgttgttcgattgatttggaatt
ctttttcgactttttgtttatattaaatattttaaaatttatagcggatagcaattcatgtaccacggataatgtagacgcgta
ttgcgcatcgatatctttattattagataaatttatcaataaatgtgagaagtttgcctcgttaaggtcttccatttaaatatt
atataaacatttgtgtttgtatcttattcgtcttttatggaatagttttttactagtaaagctgcaattacacactttgtccgt
aaaacataaatataaacaccagcttttatcaatcgttccaaaaagtcgacggcggacatttttaacatggcatctattttaaat
acacttaggtttttggaaaaaacatcattttataattgtaacgattcaataactaaagaaaagattaagattaaacataaggga
atgtcatttgtattttataagccaaagcattctaccgttgttaaatacttgtctggaggaggtatatatcatgatgatttggtt
gtattggggaaggtaacaattaatgatctaaagatgatgctattttacatggatttatcatatcatggagtgacaagtagtgga
acaatttacaaattgggatcgtctatcgatagactttctctaaataggactattgttacaaaagttaataattataattatgat
acattttttgacgatgatgattgatcgctattgcacaattttgtttttttactttctaatatagcgtttagattctttttcatg
tgcgaatattgatttactaaaatatctatgtttaacttttgttctataacgtccttatcggcggtatcggtacatatacgtaat
tcaccttcacaaaatacggagtcttcgataataatagccaatcgattattggatctagctgtctgtatcatattcaacatgttt
aatatatcctttcgtttcccctttacaggcatcgatcgtagcatattttccgcgtctgagatggaaatgttaaaactacaaaaa
tgcgtaatgttagcccgtcctaatattggtacgtgtctataagtttggcatagtagaataatagacgtgtttaaatgccttcca
aagtttaagaattctattagagtattgcattttgatagtttatcgcctacatcatcaaaaataagtaaaaagtgtgctgatttt
ttatgattttgtgcgacagcaatacatttttctatgttacttttagttcgtatcagattatattctagagattcctgactacta
acgaaattaatatgatttggccaaatgtatccatcataatctggattataaacgggtgtaaacaagaatacatgtttatatttt
ttaactagtgtagaaaacagagatagtaaatagatagtttttccagatccagatcctcccgttaaaaccattctaaacggcatt
tttaataaattttctcttgaaaattgtttttcttggaaacaattcataattatatttacagttactaaattaatttgataataa
atcaaaatatggaaaactaaggtcgttagtagggaggagaacaaagaaggcacatcgtgacataaataacatttattatcatga
tgacaccagaaaacgacgaagagcagacatctgtgttctccgctactgtttacagagacaaaattcagggaaagaataaacgca
aacgcgtgattggtctatgtattagaatatctatggttatttcactactatctatgattaccatgtccgcgtttctcatagtgc
gcctaaatcaatgcatgtctgctaacgaggctgctattactgacgccgctgttgccgttgctgctgcatcatctactcatagaa
aggttgcgtctagcactacgcaatatgatcacaaagaaagctgtaatggtttatattaccagggttcttgttatatattacatt
cagactaccagttattctcggatgctaaagcaaattgcactgcggaatcatcaacactacccaataaatccgatgtcttgacta
cctggctcattgattatgttaaggatacatggggatctgatggtaatccaattacaaaaactacatccgattatcaagattctg
atgtatcacaagaagttagaaagtatttttgtgttaaaacaatgaactaatatttatttttgtacattaataaatgaaatcgct
taatagacaaactgtaagtaggtttaagaagttgtcggtgccggtcgctataatgatgatactctcaaccattattagtggcat
aggaacatttctgcattacaaagaagaactgatgcctagtgcttgcgccaatggatggatacaatacgataaacattgttattt
agatactaacattaaaatgtctacagataatgcggtttatcagtgtcgtaaattacgagccagattgcctagaccggatactag
acatctgagagtattgtttagtattttttataaagattattgggtaagtttaaaaaagaccaatgataaatggttagatattaa
taatgataaagatatagatattagtaaattaacaaattttaaacaactaaacagtacgacggatgctgaagcgtgttatatata
caagtctggaaaactggttaaaacagtatgtaaaagtactcaatctgtactatgtgttaaaaaattctacaagtgacaacaaaa
aatgaattaataataagtcgttaacgtacgccgccatggacgccgcgtttgttattactccaatgggtgtgttgactataacag
atacattgtatgatgatctcgatatctcaatcatggactttataggaccatacattataggtaacataaaaactgtccaaatag
atgtacgggatataaaatattccgacatgcaaaaatgctactttagctataagggtaaaatagttcctcaggattctaatgatt
tggctagattcaacatttatagcatttgtgccgcatacagatcaaaaaataccatcatcatagcatgcgactatgatatcatgt
tagatatagaagataaacatcagccattttatctattcccatctattgatgtttttaacgctacaatcatagaagcgtataacc
tgtatacagctggagattatcatctaatcatcaatccttcagataatctgaaaatgaaattgtcgtttaattcttcattctgca
tatcagacggcaatggatggatcataattgatgggaaatgcaatagtaattttttatcataaaagttgtaaagtaaataataaa
acaataaatattgaactagtagtacgtatattgagcaatcagaaatgatgctggtacctcttatcacggtgaccgtagttgcgg
gaacaatattagtatgttatatattatatatttgtaggaaaaagatacgtactgtctataatgacaataaaattatcatgacaa
aattaaaaaagataaagagttctaattccagcaaatctagtaaatcaactgatagcgaatcagactgggaggatcactgtagtg
ctatggaacaaaacaatgacgtagataatatttctaggaatgagatattggacgatgatagcttcgctggtagtttaatatggg
ataacgaatccaatgttatggcgcctagcacagaacacatttacgatagtgttgctggaagcacgctgctaataaataatgatc
gtaatgaacagactatttatcagaacactacagtagtacttaatgaagataccaaacagaatcctaactattcatccaatcctt
tcgtaaattataataaaaccagtatttgtagcaagtcaaatccgttcattacagaactcaacaataaatttagtgagaataatc
cgtttagacgagcacatagcgatgattatcttaataagcaagaacaagatcatgaacacgatgatatagaatcattggtgtgat
tagtttcctttttataaaattgaagtaatatttagtattattgctgccgtcacgttgtacaaatggagatattccctgtattcg
gcatttctaaaattagcaattttattgctaataatgactgtagatattatatagatacagaacatcaaaaaattatatctgatg
agatcaatagacagatggatgaaacggtacttcttaccaacatcttaagcgtagaagttgtaaatgacaatgagatgtaccatc
ttattcctcatagattatcgacgattatactctgtattagttctgtcggaggatgtgttatctctatagataatgacgtcaatg
gcaaaaatattctaacctttcccattgatcatgctgtaatcatatccccactgagtaaatgtgtcgtagttagcaagggtccta
caaccatattggttgttaaagcggatatacctagcaaacgattggtaacatcgtttacaaacgacatactgtatgtaaacaatc
tatcactgattaattattcgccgttgtctgtattcattattagacgagttaccgactatttggatagacacatatgcgatcaga
tatttgcgaataataagtggtattccattataaccatcgacaataagcagtttcctattccatcaaactgtataggtatgtcct
ctgccaagtacataaattctagcatcgagcaagatactttaatacatgtttgtaacctcgagcatccattcgacttagtataca
aaaaaatgcagtcgtacaattctgtacctatcaaggaacaaatattgtacggtagaattgataatataaatatgagcattagta
tttctgtggattaatagatttctagtatggggatcattaatcatctctaatctctaaatacctcataaaacgaaaaaaaagcta
ttatcaaatactgtacggaatggattcattctcttctctttttatgaaactctgttgtatatctactgataaaactggaagcaa
aaaatctgataaaaagaataagaataagatcaaggattattataaaataacaatagttcctggttcctcttccacgtctactag
ctcgtggtattatacacatgcctagtaatagtctctttgcgttgacggaaagcagactagaaataacaggctaaaatgttcaga
caccataatagttcccaacccagataataacagagtaccatcaacacattcctttaaactcaatcccaaacccaaaaccgttaa
aatgtatccggccaattgatagtagataatgaggtgtacagcgcatgatgatttacacagtaaccaaaatgaaaatactttagt
aattataagaaatatagatggtaacgtcatcatcaacaatccaataatatgccggagagtaaacattgacggataaaacaaaaa
tgctccgcataactctatcatggcaataacacaaccaaatacttgtaagattcctaaattagtagaaaatacaacggatatcga
tgtataagtgatctcgagaaataataagaataaagtaatgcccgtaaagataaacatcaacattgtttggtaatcattaaacca
attagtatgaagttgaactaatttcacagtagattttattccagtattatccccgcatgtataagtacctggtaagatatcttt
atattccataatcaatgagacatcactatctgataacgaatgaagtctagcactagtatgccatttacttaatattgtcgtctt
ggaagttttattataagttaaaatatcatggttatccaatttccatctaatatactttgtcggattatctatagtacacggaat
aatgatggtatcattacatgctgtatactctatggtctttgtagttgttataacaaccaacgtatagaggtatatcaacgatat
tctaactcttgacattttttatttatttaaaatgatacctttgttatttattttattctattttgctaacggtattgaatggca
taagtttgaaacgagtgaagaaataatttctacttacttattagacgacgtattatacacgggtgttaatggggcggtatacac
attttcaaataataaactaaacaaaactggtttaactaataataattatataacaacatctataaaagtagaggatgcggaacc
aataacggaaatcccaaatgttggaaaatagacggttcagacgacccaaaacatagaggtagaggatacgctccttatcaaaat
agcaaagtaacgataatcagtcacaacggatgtgtactatctgacataaacatatcaaaagaaggaattaaacgatggagaaga
tttgacggaccatgtggttatgatttatacacggcggataacgtaattccaaaagatggtttacgaggagcattcgtcgataaa
gatggtacttatgacaaagtttacattcttttcactgatactatcggctcaaagagaattgtcaaaattccgtatatagcacaa
atgtgcctaaacgacgaaggtggtccatcatcattgtctagtcatagatggtcgacgtttctcaaagtcgaattagaatgtgat
atcgacggaagaagttatagacaaattattcattctagaactataaaaacagataatgatacgatactatatgtattcttcgat
agtccttattccaagtccgcattatgtacctattctatgaataccattaaacaatctttttctacgtcaaaattggaaggatat
acaaagcaattgccgtctccagctcctggtatatgtttaccagctggaaaagttgttccacataccacgtttgaagtcatagaa
aaatataatgtactagatgatattataaagcctttatctaaccaacctatcttcgaaggaccgtctggtgttaaatggttcgat
ataaaggagaaggaaaatgaacatcgggaatatagaatatacttcataaaagaaaattctatatattcgttcgatacaaaatct
aaacaaactcgtagctcgcaagtcgatgcgcgactattttcagtaatggtaacttcgaaaccgttatttatagcagatataggg
ataggagtaggaatgccacaaatgaaaaaaatacttaaaatgtaatcttaatcgagtacaccgcacgacaatgaacaaacataa
gacagattatgctggttatgcttgctgcgtaatatgcggtctaattgttggaattatttttacagcgacactattaaaagttgt
agaacgtaaattagttcatacaccatcaatagataaaacgataaaagatgcatatattagagaagattgtcctactgactggat
aagctataataataaatgtatccatttatctactgatcgaaaaacctgggaggaaggacgtaatgcatgcaaagctctaaatcc
aaattcggatctaattaagatagagactccaaacgagttaagttttttaagaagcattagacgcggatattgggtaggagaatc
cgaaatattaaaccagacaaccccatataattttatagctaaaaatgccacgaagaatggaactaaaaaacggaaatatatttg
tagtacaacgaatactcccaaactacatttttatcataccactacttcggttagatgttttagaaaaaaataaatatcgccgta
ccgttcttgtttttataaaaataacaattaacaattatcaaattttttctttaatattttacgtggttgaccattcttggtggt
aaaataatctcttagtgttggaatggaatgctgtttaatgtttccgcactcatcgtatattttgacgtatgcagtcacatcgtt
tacgcaatagtcagactgtagttctatcatgcttcctacatcagaaggaggaacagttttaaagtctcttggttttaatctatt
gccattagttttcatgaaatcctttgttttatccacttcacattttaaataaatgtccactatacattcttctgttaattttac
tagatcgtcatgggtcatagaatttataggttccgtagtccatggatccaaactagcaaacttcgcgtatacggtatcgcgatt
agtgtatacaccaactgtatgaaaattaagaaaacagtttaataaatcaacagaaatatttaatcctccgtttgatacagatgc
gccatatttatggatttcggattcacacgttgtttgtctgaggtgttcgtctagtgttgcttctacgtaaacttcgattcccat
atattctttattgtcagaatcgcataccgatttatcatcatacactgtttgaaaactaaatggtatacacatcaaaataataaa
taataacgagtacattctgcaatattgttatcgtaattggaaaaatagtgttcgagtgagttggattatgtgagtattggattg
tatattttattttatattttatattttgtagtaagaatagaatgctaatgtcaagtttattccaatagatgtcttattaaaaaa
catatataataaataacaatggctgaatggcataaaattatcgaggatatctcaaaaaataataagttcgaggatgccgccatc
gttgattacaagactacaaagaatgttctagctgctattcctaacagaacatttgccaagattaatcctctcatcactaatcgt
aatattctaaaacctcttattggtcagaaatattgtattgtatatactaactctctaatggatgagaacacgtatgctatggag
ttgcttactgggtacgcccctgtatctccgatcgttatagcgagaactcataccgcacttatatttttgatgggtaagccaaca
acatccagacgtgacgtgtatagaacgtgtagagatcacgctacccgtgtacgtgcaactggtaattaaaataaaaagtaatat
tcatatgtagtgtcaattttaaatgatgatgatgaaatggataatatccatattgacgatgtcaataatgccggtattggcata
cagttcatcgatttttagatttcattcagaggatgtggaattatgttatgggcatttgtattttgataggatctataatgtagt
aaatataaaatataatccgcatattccatatagatataattttattaatcgcacgttaaccgtagatgaactagacgataatgt
cttttttacacatggttattttttaaaacacaaatatggttcacttaatcctagtttgattgtctcattatcaggaaacttaaa
atataatgatatacaatgctcagtaaatgtatcgtgtctcattaaaaatttggcaacgagtacatctactatattaacatctaa
acataagacttattctctacatcggtccacgtgtattactataataggatacgattctattatatggtataaagatataaatga
catctatgattttactgcaatatgtatgctaatagcgtctacattgatagtgaccatatacgtgtttaaaaaaataaaaatgaa
ctcttaattatgctatgctattagaaatggataaaatcaaaattacggttgattcaaaaattggtaatgttgttaccatatcgt
ataacttggaaaagataactattgatgtcacacctaaaaagaaaaaagaaaaggatgtattattagcgcaatcagttgctgtcg
aagaggcaaaagatgtcaaggtagaagaaaaaaatattatcgatattgaagatgacgatgatatggatgtagaaagcgcataat
acgatctataaaaataagtatataaatactttttatttactgtactcttactgtgtagtggtgataccctactcgattattttt
ttaaaaaaaaatacttattctgattcttctagccatttccgtgttcgttcgaatgccacatcgacgttaaagataggggagtag
ttgaaatctagttctgcattgttggtacgcacctcaaatgtagtgttggatatcttcaacgtatagttgttgagtagtgatggt
tttctaaatagaattctcttcatatcattcttgcacgcgtacatttttagcatccatcttggaatcctagatccttgttctatt
cccaatggtttcatcaatagaagattaaacatatcgtacgaacacgatggagagtaatcgtagcaaaagtaagcatttccttta
atctcagatcccggatactggatatattttgcagccaacacgtgcatccatgcagcatttcctacatatacccggctatgtacc
gcgttatcatcgactgtacgatacataatgttaccgtgttgcttacattgctcgtaaaagactttcatcaatttgtctccttct
ccgtaaattccagtgggtcttaggcaacaagtatacaattttgctccattcatgattacggaattattggctttcataaccagt
tgctcggccatacgtttactttttgcgtatacatgtcctggtgatatatcataaagggtatgctcatggccgatgaatggatca
ccgtgtttattgggtcctattgcttccatgctactagtatagatcaaatacttgattcctaggtccacacaagctgccaatata
gtctgtgttccataatagtttactttcatgatttcattatcggtgtattttccaaatacatccactagagcagctgtatgaata
atcagatttaccccatctagcgcttctcttaccttatcaaagtcgtttatatcacattgtatatagtttataaccttaactttc
gaggttattggttgtggatcttctacaatatctatgactctgatttcttgaacatcatctgcactaattaacagttttactata
tacctgcctagaaatccggcaccaccagtaaccgcgtacacggccattgctgccactcataatatcagactacttattctattt
tactaaataatggctgtttgtataatagaccacgataatatcagaggagttatttactttgaaccagtccatggaaaagataaa
gttattggattaaaatccggaacgtatagtttgataattcatcgttacggagatattagtcaaggatgtgattccataggcagt
ccagaaatatttatcggtaacatctttgtaaacagatatggtgtagcatatgtttatttagatacagatgtaaatatatctaca
attattggaaaggcgttatctatttcaaaaaatgatcagagattagcgtgtggagttattggtatttcttacataaatgaaaag
ataatacattttcttacaattaacgagaatggcgtttgatatatcagttaatgcgtctaaaacaataaatgcattagtttactt
ttctactcagcaaaataaattagtcatacgtaatgaagttaatgatacacactacactgtcgaatttgatagggacaaagtagt
tgacacgtttatttcatataatagacataatgactccatagagataagaggggtgcttccagaggaaactaatattggttgcgc
ggttaatacgccggttagtatgacttacttgtataataagtatagttttaaactgattttagcagaatatataagacacagaaa
tactatatccggcaatatttattcggcattgatgacactagatgatttggctattaaacagtatggagacattgatctattatt
taatgagaaacttaaagtagactccgattcgggactatttgactttgtcaactttgtaaaggatatgatatgttgtgattctag
aatagtagtagctctatctagtctagtatctaaacattgggaattgacaaataaaaaatataggtgtatggcattagccgaaca
tatatctgatagtattccaatatctgagctatctagactacgatacaatctatgtaagtatctacgcgggcacactgagagcat
agaggatgaatttgattattttgaagacgatgattcgtctacatgttctgccgtaaccgacagggaaacggatgtataattttt
tttatagcgtgaaggatatgataaaaaatataattgttgtatttatcccattccaatcaccttatatgattctgtaacacaatg
aaggagtcttatagatgtatagaggtcagatactggtttgataaactgtttattccacataagtatgtttgactttatggttag
acccgcatactttaacaaatcactgaaaattggagttaggtattgacctctcagaatcagttgccgttctggaacattaaatgt
attttttatgatatactccaacgcatttatgtgggcatacaacaagtcattactaatggagtattccaagagaagagatttcaa
cagactgtttatgaactcgaatgccgcctcattgtcgcttatattgatgatgtcgaattctcccaatatcatcaccgatgagta
gctcatcttgttatcgggatccaagttttctaaagatgtcattaaaccctcgatcatgaatggatttatcatcatcgtttttat
gttggacatgagcttagtccgtttgtccacatctatagacgacgatttctgaattatttcatatatccctctctttaactccag
gaacttgtcaggatggtctactttaatatgttctcgtctaagagatgaaaatctttggatggttgcacgcgacttttctctaaa
ggatcctctcttaaatgaatccatcttatccttggacaagatggacagtctattttccttagatggtttaatatttttgttacc
catgatctataaaggtagacctaatcgtctcggatgaccatatatttattttcagttttattatacgcataaattgtaaaaaat
atgttaggtttacaaaaatgtctcgtggggcattaatcgtttttgaaggattggacaaatctggaaaaacaacacaatgtatga
acatcatggaatctataccggcaaacacgataaaatatcttaactttcctcagagatccactgtcactggaaagatgatagatg
actatctaactcgtaaaaaaacctataatgatcatatagttaatctattattttgtgcaaatagatgggagtttgcatctttta
tacaagaacaactagaacagggaattactttaatagttgatagatacgcattctctggagtagcgtatgccgccgctaaaggcg
cgtcaatgactctcagtaagagttatgaatctggattgcctaaacccgacttagttatattcttggaatctggtagcaaagaaa
ttaatagaaacgtcggcgaggaaatttatgaagatgttacattccaacaaaaggtattacaagaatataaaaaaatgattgaag
aaggagatattcattggcaaattatttcttctgaattcgaggaagatgtaaagaaggagttgattaagaatatagttatagagg
ctatacacacggttactggaccagtggggcaactgtggatgtaatagtgaaattacattttttataaatggatgaagcatatta
ctctggcaacttggaatcagtactcggatacgtgtccgatatgcataccgaactcgcatcaatatctcaattagttattgccaa
gatagaaactatagataatgatatattaaacaaggacattgtaaattttatcatgtgtagatcaaacttggataatccatttat
ctctttcctagatactgtatatactattatagatcaagagatctatcagaccgaattgattaattcattagacgacaatgaaat
tatcgattgtatagttaacaagtttatgagcttttataaggataacctagaaaatatagtagatgctatcattactctaaaata
tataatgaataatccagattttaaaactacgtatgccgaagtactcggttccagaatagcggatatagatattaaacaagtgat
acgtgagaatatactacaattgtctaataatatccgcgaacgatatttgtgaaaatattaaaaaaaaatactttttttattaaa
tgacgtcgcttcgcgaatttagaaaattatgctgtgatatatatcacgcatcaggatataaagaaaaatctaaattaattagag
actttataacagatagggatgataaatatttgatcattaagctattgcttcccggattagacgatagaatttataacatgaacg
ataaacaaattataaaattatatagtataatatttaaacaatctcaggaagatatgctacaagatttaggatacggatatatag
gagacactattaggactttcttcaaagagaacacagaaatccgtccacgagataaaagcattttaactttagaagaagtggata
gttttttaactacgttatcatccgtaactaaagaatcgcatcaaataaaattattgactgatatcgcatccgtttgtacatgta
atgatttaaaatgtgtagtcatgcttattgataaagatctaaaaattaaagcgggccctcggtacgtacttaacgctattagtc
ctcatgcctatgatgtgtttagaaaatctaataacttgaaagagataatagaaaatgcatctaaacaaaatctagactctatat
ctatttctgttatgactccaattaatcccatgttagcggaatcgtgtgattctgtcaataaggcgtttaaaaaatttccatcag
gaatgtttgcggaagtcaaatacgatggtgaaagagtacaagttcataaaaataataacgagtttgccttctttagtagaaaca
tgaaaccagtactctctcataaagtggattatctcaaagaatacataccgaaagcatttaaaaaagctacgtctatcgtattgg
attctgaaattgttcttgtagacgaacataatgtaccgctcccgtttggaagtttaggtatacacaaaaagaaagaatataaaa
actctaacatgtgtttgttcgtgtttgactgtttgtactttgatggattcgatatgacggacattccattgtacgaacgaagat
cttttctcaaagatgttatggttgaaatacccaatagaatagtattctcagagttgacgaatattagtaacgagtctcagttaa
ctgacgtattggatgatgcactaacgagaaaattagaaggattggtcttaaaagatattaatggagtatacgaaccgggaaaga
gaagatggttaaaaataaagcgagactatttgaacgagggttccatggcagattctgccgatttagtagtactaggtgcttact
atggtaaaggagcaaagggtggtatcatggcagtctttctaatgggttgttacgacgatgaatccggtaaatggaagacggtta
ccaagtgttcaggacacgatgataatacgttaagggagttgcaagaccaattaaagatgattaaaattaacaaggatcccaaaa
aaattccagagtggttagtagttaataaaatctatattcccgattttgtagtagaggatccaaaacaatctcagatatgggaaa
tttcaggagcagagtttacatcttccaagtcccataccgcaaatggaatatccattagatttcctagatttactaggataagag
aggataaaacgtggaaagaatctactcatctaaacgatttagtaaacttgactaaatcttaatagttacatacaaattaaaata
acactatttagttggtggtcgccatggatggtgttattgtatactgtctaaacgcgttagtaaaacatggcgaggaaataaatc
atataaaaaatgatttcatgattaaaccatgttgtgaaaaagtcaagaacgttcacattggcggacaatctaaaaacaatacag
tgattgcagatttgccatatatggataatgcggtatccgatgtatgcaattcactgtataaaaagaatgtatcaagaatatcca
gatttgctaatttgataaagatagatgacgatgacaagactcctactggtgtatataattattttaaacctaaagatgccattc
ctgttattatatccataggaaaggatagagatgtttgtgaactattaatctcatctgataaagcgtgtgcgtgtatagagttaa
attcatataaagtagccattcttcccatggatgtttccttttttaccaaaggaaatgcatcattgattattctcctgtttgatt
tctctatcgatgcggcacctctcttaagaagtgtaaccgataataatgttattatatctagacaccagcgtctacatgacgagc
ttccgagttccaattggttcaagttttacataagtataaagtccgactattgttctatattatatatggttgttgatggatctg
tgatgcatgcaatagctgataatagaacttacgcaaatattagcaaaaatatattagacaatactacaattaacgatgagtgta
gatgctgttattttgaaccacagattaggattcttgatagagatgagatgctcaatggatcatcgtgtgatatgaacagacatt
gtattatgatgaatttacctgatgtaggcgaatttggatctagtatgttggggaaatatgaacctgacatgattaagattgctc
tttcggtggctggtatttggaaagttttataggtagttgatagaacaaaatacataattttgtaaaaataaatcactttttata
ctaatatgacacgattaccaatacttttgttactaatatcattagtatacgctacaccttttcctcagacatctaaaaaaatag
gtgatgatgcaactttatcatgtaatcgaaataatacaaatgactacgttgttatgagtgcttggtataaggagcccaattcca
ttattcttttagctgctaaaagcgacgtcttgtattttgataattataccaaggataaaatatcttacgactctccatacgatg
atctagttacaactatcacaattaaatcattgactgctagagatgccggtacttatgtatgtgcattctttatgacatcgccta
caaatgacactgataaagtagattatgaagaatactccacagagttgattgtaaatacagatagtgaatcgactatagacataa
tactatctggatctacacattcaccggaaactagttctgagaaacctgattatatagataattctaattgctcgtcggtattcg
aaatcgcgactccggaaccaattactgataatgtagaagatcatacagacaccgtcacatacactagtgatagcattaatacag
taagtgcatcatctggagaatccacaacagacgagactccggaaccaattactgataaagaagaagatcatacagttacagaca
ctgtctcatacactacagtaagtacatcatctggaattgtcactactaaatcaaccaccgatgatgcggatctttatgatacgt
acaatgataatgatacagtaccatcaactactgtaggcggtagtacaacctctattagcaattataaaaccaaggactttgtag
aaatatttggtattaccgcattaattatattgtcggccgtggcaatattctgtattacatattatatatataataaacgttcac
gtaaatacaaaacagagaacaaagtctagatttttgacttacataaatgtctgggatagtaaaatctatcatattgagcgggcc
atctggtttaggaaagacagccatagccaaaagactatgggaatatatttggatttgtggtgtcccataccactagatttcctc
gtcctatggaacgagaaggtgtcgattaccattacgttaacagagaggccatctggaagggaatagccgccggaacatactgag
tttttaggaaatatttacggaacttctaaaactgctgtgaatacagcggctattaataatcgtatttgtgtgatggatctaaac
atcgacggtgttagaagttttaaaaatacttacctaatgccttactcggtgtatataagacctacctctcttaaaatggttgag
accaagcttcgttgtagaaacactgaagcggatgatgagattcatcgtcgtgtgatgttggcaaaaactgacatggatgaggca
ggtgaagccggtctattcgacactattatcattgaagatgatgtgaatttagcatatagtaagttaattcagatactacaggac
cgtattagaatgtattttaacactaattagagacttaagacttaaaacttgataattaataatataactcgtttttatatgtgg
ctatttcaacgtctaatgtattagttaaatattaaaacttaccacgtaaaacttaaaatttaaaatgatatttcattgacagat
agatcacacattatgaactttcaaggacttgtgttaactgacaattgcaaaaatcaatgggtcgttggaccattaataggaaaa
ggtggatttggtagtatttatactactaatgacaataattatgtagtaaaaatagagcccaaagctaacggatcattatttacc
gaacaggcattttatactagagtacttaaaccatccgttatcgaagaatggaaaaaatctcacaatataaagcacgtaggtctt
atcacgtgcaaggcatttggtctatacaaatccattaatgtggaatatcgattcttggtaattaatagattaggtgcagatcta
gatgcggtgatcagagccaataataatagattaccaaaaaggtcggtgatgttgatcggaatcgaaatcttaaataccatacaa
tttatgcacgagcaaggatattctcacggagatattaaagcgagtaatatagtcttggatcaaatagataagaataaattatat
ctagtggattacggattggtttctaaattcatgtctaatggcgaacatgttccatttataagaaatccaaataaaatggataac
ggtactctagaatttacacctatagattcgcataaaggatacgttgtatctagacgtggagatctagaaacacttggatattgt
atgattagatggttgggaggtatcttgccatggactaagatatctgaaacaaagaattgtgcattagtaagtgccacaaaacag
aaatatgttaacaatactgcgactttgttaatgaccagtttgcaatatgcacctagagaattgctgcaatatattaccatggta
aactctttgacatattttgaggaacccaattacgacaagtttcggcacatattaatgcagggtgtatattattaagtgtggtgt
ttggttgatgtaaaatttttgtcgataaaaattaaaaaataacttaatttattattgatctcgtgtgtacaaccgaaatcatgg
cgatgttttacgcacacgctctcggtgggtacgacgagaatcttcatgcctttcctggaatatcatcgactgttgccaatgatg
tcagtttataataacaagtatgacattgtaaaagacaaatatatgtggtgttacagtcaggtgaacaagagatatattggagca
ctgctgcctatgtttgagtgcaatgaatatctacaaattggagatccgatccatgatcaagaaggaaatcacatatcgccacaa
aaactactatgctctaagcggaatcgggtacgagagtctagacttgtgtttggaaggagtagggattcatcatcacgtacttga
aacagaaaacgctgtatatggaaaagttcaacatgattattctactatcaaagagaaggccaaagaaatgaatgcactcagttc
aggacctatcatcgattaccacgtctggataggagattgtatctgtcaagttactgctgtggacgtacatggaaaggaaattat
gagaatgagattcaaaaagggtgcggtgcttccgatcccaaatctggtaaaagttaaacttggggagaatgatacagaaaatct
ttcttctactatatcggcggcaccatcgaggtaaccacctctctggaagacagtgtgaatcatgtactcatgaaacgtttggaa
tctatacgccatatgtggtctgttgtatatgatcattttgatattgtgaatggtaaagaatgctgttatgtgcatacgcatttg
tctaatcaaaatcttataccgagtactgtaaaaacaaatttgtacatgaagactatgggatcatgcattcaaatgagtatctta
gcgaactgaaggaatcaggtggatggagtcccagaccagaaatgcaggaatttgaatatccagatggagtggaagacactgaat
caattgagagattggtagaggagttcttcaatagatcagaacttcaggctggtaaatctattaatgttaaacatacatctgttt
cagctaagcaactaagaacacgtatactctcatcttttgccaacacagagggtggatatttgttcattggagttgataataata
cacacaaagtatttggattcacggtgggttacgactacctcagactgatagagaatgatatagaaaagcatatcaaaagacttt
gtgttgtgtatttctgtgagaagaaagaggacatcaagtacacgtgtcgattcatcaaggtatataaacctggggatgaggcta
cctcgacatacgtgtgcgctatcaaagtggaaagatgctgttgtgctgtgtttgcagattggccagaatcatggtatcaagaag
tattctccagatgaataggtgtcacatataaaattttaattaatgtaactatagagaacaaataataggttgtaatatcatata
gacaataactaacaattaattagtaactgttatctctttttaactaactaactatacctattaatacatcgtaattatagttct
taacatctattaatcattgattcgcttctttaattttttataaaccaacattgttaattgaaaagggataacatgttacagaat
ataaattatatatggattttttttaaaaaggaaatacttgactggagtgtatatttatctcttcattatatagcacgcgtgttt
tccaatttttccacatcccatataatacaggattataatctcgttcgaacatacgagaaagtggataaaacaatagttgatttt
ttatctaggttgccaaatttattccatattttagaatatggggaaaatattctacatatttattctatggatgatgctaatacg
aatattataattttttttctagatagagtattaaatattaataagaacgggtcatttatacacaatctcaggttatcatcatcc
attaatataaaagaatatgtatatcaattagttaataatgatcatccagataataggataagactaatgcttgaaaatggacgt
agaacaagacattttttgtcctatatatcagatacagttaatatctatatatgtattttatatagatgccgaagacagttacgg
ttgtacattattacatagatgtatatatcactataagaaatcagaatcagaatcagaatcatacaatgaattaattaagatatt
gttaaataatggatcagatgtagataaaaaagatacgtacggaaacacaccttttatcctattatgtaaacacgatatcaacaa
cgtggaattgtttgagatatgtttagagaatgctaatatagactctgtagactttaatagatatacacctcttcattatgtctc
atgtcgtaataaatatgattttgtaaagttattaatttctaaaggagcaaatgttaatgcgcgtaataaattcggaactactcc
attttattgtggaattatacacggtatctcgcttataaaactatatttggaatcagacacagagttagaaatagataatgaaca
tatagttcgtcatttaataatttttgatgctgttgaatctttagattatctattatccagaggagttattgatattaactatcg
tactatttacgacgctgtcagttataatgcgtataatacgttggtctatctattaaacagaaatggtgattttgagacgattac
tactagtggatgtacatgtatttcggaagcagtcgcaaacaacaacaaaataataatggaagtactattgtctaaacgaccatc
tttgaaaattatgatacagtctatgatagcaattactaaacataaacagcataatgcagatttattgaaaatgtgtataaaata
tactgcgtgtatgaccgattatgatactcttatagatgtacaatcactacagcaatataaatggtatattttaagatgtttcga
tgaaatagatatcatgaagagatgttatataaaaaataaaactgtattccaattagttttttgtatcaaagacattaatacttt
aatgagatacggtaaacatccttctttcgtgaagtgcactagtctcgacgtatacggaagtcgtgtacgtaatatcatagcatc
tattagatatcgtcagagattaattagtctattatccaagaagctggatcctggagataaatggtcgtgttttcctaacgaaat
aaaatataacgataacgaactgtccacatatctaaaaatcttataaacactattaaaatataaaatcacactacatcattgttt
ccttttagtgctcgacagtgtatactatttttaacgctcataaataaaaatgaaaacgatttccgttgttacgttgttatgcgt
actacctgctgttgtttattcaacatgtactgtacccactatgaataacgctaaattaacgtctaccgaaacatcgtttaataa
taaccagaaagttacgtttacatgtgatcagggatatcattcttcggatccaaatgctgtctgcgaaacagataaatggaaata
cgaaaatccatgcaaaaaaatgtgcacagtttctgattacatctctgaactatataataaaccgctatacgaagtgaattccac
catgacactaagttgcaacggcgaaacaaaatattttcgttgcgaagaaaaaaatggaaatacttcttggaatgatactgttac
gtgtcctaatgcggaatgtcaacctcttcaattagaacacggatcgtgtcaaccagttaaagaaaaatactcatttggggaata
tataactatcaactgtgatgttggatatgaggttattggtgcttcgtacataagttgtacagctaattcttggaatgttattcc
atcatgtcaacaaaaatgtgatataccgtctctatctaatggattaatttccggatctacattttctatcggtggcgttataca
tcttagttgtaaaagtggttttatactaacgggatctccatcatccacatgtatcgacggtaaatggaatcccatactcccaac
atgtgtacgatctaacgaaaaatttgatccagtggatgatggtcccgacgatgagacagatttgagcaaactctcgaaagacgt
tgtacaatatgaacaagaaatagaatcgttagaagcaacttatcatataatcatagtggcgttaacaattatgggcgtcatatt
tttaatctccgttatagtattagtttgttcctgtgacaaaaataatgaccaatataagttccataaattgctaccgtgaatata
aatccgttaaaataatgaataattaataattaataatttaataacaaacaagtatcaaaagattaaagacttatagctagaatc
aattgagatgtcttcttcagtggatgttgatatctacgatgccgttagagcatttttactcaggcactattataacaagagatt
tattgtgtatggaagaagtaacgccatattacataatatatacaggctatttacaagatgcgccgttataccgttcgatgatat
agtacgtactatgccaaatgaatcacgtgttaaacaatgggtgatggatacacttaatggtataatgatgaatgaacgcgatgt
ttctgtaagcgttggcaccggaatactattcatggaaatgtttttcgattacaataaaaatagtatcaacaatcaactaatgta
tgatataattaatagcgtatctataattctagctaatgagagatatagaagcgcttttaacgacgatggtatatacatccgtag
aaatatgattaacaagttgtacggatacgcatctctaactactattggcacgatcgctggaggtgtttgttattatctgttgat
gcatctagttagtttgtataaataattatttcaatatactagttaaaattttaagattttaaatgtataaaaaactaataacgt
ttttatttgtaataggtgcattagcatcctattcgaataatgagtacactccgtttaataaactgagtgtaaaactctatatag
atggagtagataatatagaaaattcatatactgatgataataatgaattggtgttaaattttaaagagtacacaatttctatta
ttacagagtcatgcgacgtcggatttgattccatagatatagatgttataaacgactataaaattattgatatgtctactattc
aacgcagaggtcacacgtgtagaatatctaccaaattatcatgccattatgataagtacccttatattcacaaatatgatggtg
atgagcgacaatattctattactgcagagggaaaatgctataaaggaataaaatatgaaataagtatgatcaacgatgatactc
tattgagaaaacatactcttaaaattggatctacttatatatttgatcgtcatggacatagtaatacatattattcaaaatatg
atttttaaaaatttaaaatatattatcacttcagtgacagtagtcaaataacaaacaacaccatgagatatattataattctcg
cagttttgttcattaatagtatacacgctaaaataactagttataagtttgaatccgtcaattttgattccaaaattgaatgga
ctggggatggtctatacaatatatcccttaaaaattatggcatcaagacgtggcaaacaatgtatacaaatgtaccagaaggaa
catacgacatatccgcatttccaaagaatgatttcgtatctttctgggttaaatttgaacaaggcgattataaagtggaagagt
attgtacgggaccaccgactgtaacattaactgaatacgacgaccatccgtatgctactagaggtagcaaaaagattcctattt
acaaacgcggtgacatgtgtgatatctacttgttgtatacggctaacttcacattcggagattctaaagaaccagtaccatatg
atatcgatgactacgattgcacgtctacaggttgcagcatagactttgtcacaacagaaaaagtgtgcgtgacagcacagggag
ccacagaagggtttctcgaaaaaattactccatggagttcgaaagtatgtctgacacctaaaaagagtgtatatacatgcgcaa
ttagatccaaagaagatgttcccaatttcaaggacaaaatggccagagttatcaagagaaaatttaactaaatttctcggtagc
acatcaaatgatgttaccacttttcttagcatgcttaacttgactaaatattcataactaatttttattaatgatacaaaaacg
aaataaaactgcatattatacactggttaacgcccttataggctctaaccattttcaagatgaggtccctgattatagtccttc
tgttcccctctatcatctactccatgtctattagacgatgtgagaagactgaagaggaaacatggggattgaaaatagggttgt
gtataattgccaaagatttctatcccgaaagaactgattgcagtgttcatctcccaactgcaagtgaaggcaatggattcaggg
atatacgaaacaccgataaattataaaaaaagcaatgtgtccgctgtttccgttaataatactattttcgtaactggcggatta
ttcataaataactctaatagcacgatcgtggttaacaatatggaaaaacttgacatttataaagacaaacaatggtcgattata
gaaatgcctatggctagggtatatcacggcatcgactcgacatttggaatgttatattttgccggaggtctatccgttaccgaa
caatatggtaatttagagaaaaacaacgagatatcttgttacaatcctagaacgaataagtggtttgatatttcatatactatt
tataagatatccatatcatcattgtgtaaactaaataacgtcttctatgtatttagtaaggacattggatatgtggaaaagtat
gatggtctccccgctataaaggcattatcaacttctccttattgattgaaaatgaaaatataaatagtttttatgtatagcagt
attaccctatagttttattgcttactactaacatggatacagatacagatgttacaaatgtagaagatatcatgaatgaaatag
atagagagaaagaagaaatactaaaaaatgtagaaattgaaaataataaaaacattaacaagaatcatcccaatgaatatatta
gagaagcactcgttattaatacaagtagtaatagtgattccattgataaagaagttatagaatgtatcagtcacgatgtaggaa
tatagatcatatctactaatttttataatcaatacaaaacataaaaaacaactcgttattacatagcaggcatggaatccttca
agtattgttttgataacgatggcaagaaatggattatcggaaatactttatattctggtaattcaatactctataaggtcagaa
aaaatttcactagttcgttctacaattacgtaatgaagatagatcacaaatcacacaagccattgttgtctgaaatacgattct
atatatctgtattggatcctttgactatcgacaactggacacgggaacgtggtataaagtatttggctattccagatctgtatg
gaattggagaaaccgatgattatatgttcttcgttataaagaatttgggaagagtattcgccccaaaggatactgaatcagtct
tcgaagcatgcgtcactatgataaacacgttagagtttatacactctcgaggatttacccatggaaaaatagaaccgaggaata
tactgattagaaataaacgtctttcactaattgactattctagaactaacaaactatacaagagtggaaactcacatatagatt
acaacgaggacatgataacttcaggaaatatcaattatatgtgtgtagacaatcatcttggagcaacagtttcaaaacgaggag
atttagaaatgttgggatattgcatgatagaatggttcggtggcaaacttccatggaaaaacgaaagtagtataaaagtaataa
aacaaaaaaaagaatataaaaaatttatagctactttctttgaggactgttttcctgaaggaaatgaacctctggaattagtta
gatatatagaattagtatacacgttagattattctcaaactcctaattatgacagactacgtagactgtttatacaagattgaa
atattctttttttatagagtgtggtagtgttacggatatctaatattaatattagactatctctatcgcgctacacgaccaata
tcgattactatggatatcttctatgaaaggagagaatgtattcatttctccagcgtcaatctcgtcagtattgacaatactgta
ttatggagctaatggatccactgctgaacagctatcaaaatatgtagaaaaggaggagaacacggataaggttagcgctcagaa
tatctcattcaaatccatgaataaagtatatgggcgatattctgccgtgtttaaagattcctttttgagaaaaattggcgataa
gtttcaaactgttgacttcactgattgtcgcactatagatgcaatcaacaagtgtgtagatatctttactgaggggaaaatcaa
tccactattggatgaacaattgtctcctagcaattagtgccgtatactttaaagcaaaatggttgacgccattcgaaaaggaat
ttaccagtgattatcccttttacgtatcaccaacggaaatggtagacgtaagtatgatgtctatgtacggcgagctatttaatc
acgcatctgtaaaagaatcattcggtaacttttcaatcatagaactgccatatgttggagatactagtatgatggtcattcttc
cagacaagattgatggattagaatccatagaacaaaatctaacagatacaaattttaagaaatggtgtaactctctggaagcta
cgtttatcgatgttcacattcccaagtttaaggtaacaggctcgtataatctggtggatactctagtaaagtcaggactgacag
aggtgttcggttcaactggagattatagcaatatgtgtaatttagatgtgagtgtcgacgctatgatccacaaaacgtatatag
atgtcaatgaagagtatacagaagcagctgcagcaacttgtgcactggtgtcagactgtgcatcaacaattacaaatgagttct
gtgtagatcatccgttcatctatgtgattaggcatgttgatggaaaaatttttttcgttggtagatattgctctccgacaacta
attgttaaccattttttttaaaaaaatagaaaaaacatgtggtattagtgcaggtcgttattcttccaattgcaattggtaaga
tgacggccaactttagtacccacgtcttttcaccacagcactgtggatgtgacagactgaccagtattgatgacgtcaaacaat
gtttgactgaatatatttattggtcgtcctatgcataccgcaacaggcaatgcgctggacaattgtattccacactcctctctt
ttagagatgatgcggaattagtgttcatcgacattcgcgagctggtaaaaaatatgccgtgggatgatgtcaaagattgtgtag
aaatcatccgttgttatataccggatgagcaaaaaaccatcatcggactttgtgcatatgctgctacttactggggaggtgaag
accatcccactagtaacagtctgaacgcattgtttgtgatgcttgagatgctaaattacgtggattataacatcatattccggc
gtatgaattgatgagttgtacatcttgacattttcttctttcttctcttctccctttcccagaaacaaactttttttacccact
ataaaataaaatgagtatactacctgttatatttctttctatatttttttattcttcattcgttcagacttttaacgcgcctga
atgtatcgacaaagggcaatattttgcatcattcatggagttagaaaacgagccagtaatcttaccatgtcctcaaataaatac
gctatcatccggatataatatattagatattttatgggaaaaacgaggagcggataatgatagaattataccgatagataatgg
tagcaatatgctaattctgaacccgacacaatcagactctggtatttatatatgcattaccacgaacgaaacctactgtgacat
gatgtcgttaaatttgacaatcgtgtctgtctcagaatcaaatatagatcttatctcgtatccacaaatagtaaatgagagatc
tactggcgaaatggtatgtcccaatattaatgcatttattgctagtaacgtaaacgcagatattatatggagcgggcatcgacg
ccttagaaataagagacttaaacaacggacacctggaattattaccatagaagatgttagaaaaaatgatgctggttattatac
atgtgttttagaatatatatacggtggcaaaacatataacgtaaccagaattgtaaaattagaggtacgggataaaataatacc
ttctactatgcaattaccagaaggtgttgtaacttcaataggtagtaatttgactattgcgtgtagagtatcgttgagacctcc
cacaacggatgcagacgtcttttggataagtaatggtatgtattacgaagaagatgatggggacggagacggtagaataagtgt
agcaaataaaatctatatgaccgataagagacgtgttattacatcccggttaaacattaatcctgtcaaggaagaagatgctac
aacgtttacgtgtatggcgtttactattcctagcatcagcaaaacagttactgttagtataacgtgaatgtatgttgttacatt
tccatgtcaattgagtttataagaatttttatacattatcttccaacaaacaattgacgaacgtattgctatgattaactccca
cgatactatgcatattattaatcattaacttgcagactatacctagtgctattttgacatactcatgttcttgtgtaattgcgg
tatctatattattaaagtacgtaaatctagctatagttttattatttaattttagataatataccgtctccttatttttaaaaa
ttgccacatcctttattaaatcatgaatgggaatttctatgtcatcgttaatatattgtgaacaacaagagcagatatctatag
gaaagggtggaatgcgatacattgatctatgtagttttaaaacacacgcgaactttgaagaatttatataaatcattccatcga
tacatccttctatgttgacatgtatatatccaggaattcttttattaatgtcaggaaatgtataaactaaaacattgcccgaaa
gcggtgcctctatctgcgttatatccgttcttaacttacaaaatgtaaccaatacctttgcatgacttgttttgttcggcaacg
ttagtttaaacttgacgaatggattaattacaatagcatgatccgcgcatctattaagtttttttactttaacgcccttgtatg
tttttacagagactttatctaaatttctagtgcttgtatgtgttataaatataacgggatatagaactgaatcacctaccttag
atacccaattacattttatcagatccagataataaacaaattttgtcgccctaactaattctatattgttatatattttacaat
tggttatgatatcatgtaataacttggagtctaacgcgcatcgtcgtacgtttatacaattgtgatttagtgtagtatatctac
acatgtatttttccgcactatagtattctggactagtgataaaactatcgttatatctgtcttcaatgaactcatcgagatatt
gctctctgtcatattcatacacctgcataaactttctagacatcttacaatccgtgttattttaggatcatatttacatattta
cgggtatatcaaagatgttagattagttaatgggaatcgtctataataatgaatattaaacaattatatgaggacttttaccac
aaagcatcataaaaatgagtcgtcgtctgatttatgttttaaatatcaaccgcgaatcaactcataaaatacaagagaatgaaa
tatatacatattttagtcattgcaatatagaccatacttctacagaacttgattttgtagttaaaaactatgatctaaacagac
gacaacctgtaactgggtatactgcactacactgctatttgtataataattactttacaaacgatgtactgaagatattattaa
atcatggagtggatgtaacgatgaaaaccagtagcggacgtatgcctgtttatatattgcttactagatgttgtaatatttcac
atgatgtagtgatagatatgatagacaaagataaaaaccacttatcgcatagagactattccaacctactactagagtatataa
aatctcgttacatgttattgaaggaagaggatatcgatgagaacatagtatccactttattagataagggaatcgatcctaact
ttaaacaagacggatatacagcgttacattattattatttgtgtctcgcacacgtttataaaccaggtgagtgtagaaaaccga
taacgataaaaaaggccaagcgaattatttctttgtttatacaacatggagctaatctaaacgcgttagataattgtggtaata
caccattccatttgtatcttagtattgaaatgtgtaataatattcatatgactaaaatgctgttgacttttaatccgaatttca
aaatatgtaataatcatggattaacgcctatactatgttatataacttccgactacatacaacacgatattcttgttatgttaa
tacatcactatgaaacaaatgttggagaaatgccgatagatgagcgtcgtataatcgtattcgagtttatcaaaacatattcta
cacgtccggcagattcgataacttatttgatgaataggtttaaaaatatagatatttatacccgctatgaaggaaagacattat
tacacgtagcatgtgaatataataatacacacgtaatagattatcttatacgtatcaacggagatataaatgcgttaaccgaca
ataacaaacacgctacacaactcattatagataacaaagaaaattccccatataccattaattgtttactgtatatacttagat
atattgtagataagaatgtgataagatcgttggtggatcaacttccatctctacctatcttcgatataaaatcatttgagaaat
tcatatcctactgtatacttttagatgacacattttacaatagacacgttaggaatcgcaattctaaaacgtatcgatacgcat
tttcaaaatacatgtcgtttgataaatacgatggtataataactaaatgtcataaagaaacaatattgctcaaactatccactg
ttctagacactacactatatgcagttttaagatgccataattcgaaaaagttaagaagatacctcaacgagttaaaaaaatata
ataacgataagtcctttaaaatatattctaatattatgaatgagagataccttaatgtatattataaagatatgtacgtgtcaa
aggtatatgataaactatttcctgttttcacagataaaaattgtctactaacattactaccttcagaaattatatacgaaatat
tatacatgctgacaattaacgatctttataatatatcgtatccacctaccaaagtatagttgtatttttctcatgcgatgtgtg
taaaaaaactgatattatataaatattttagtgccgtataatgaagatgacgatgaaaatgatggtacatatatatttcgtatc
attattgttattgctattccacagttacgccatagacatcgaaaatgaaatcacagaattcttcaataaaatgagagatactct
accagctaaagactctaaatggttgaatccagcatgtatgttcggaggcacaatgaatgatatagccgctctaggagagccatt
cagcgcaaagtgtcctcctattgaagacagtcttttatcgcacagatataaagactatgtggttaaatgggagaggctagaaaa
aaatagacggcgacaggtttctaataaacgtgttaaacatggtgatttatggatagccaactatacatctaaattcagtaaccg
taggtatttgtgtaccgtaactacaaagaatggtgactgtgttcagggtatagttagatctcatattaaaaaacctccttcatg
cattccaaaaacatatgaactaggtactcatgataagtatggcatagacttatactgtggaattctttacgcaaaacattataa
taatataacttggtataaagataataaggaaattaatatcgacgatattaagtattcacaaacgggaaagaaattaattattca
taatccagagttagaagatagtggaagatacaactgttacgttcattacgacgacgttagaatcaagatgtaaaatacttacgg
ttataccgtcgcaagaccacaggtttaaactaatactagatccaaaaatcaacgtaacgataggagaacctgccaatataacat
gcactgctgtgtcaacgtcattattgattgacgatgtactgattgaatgggaaaatccatccggatggcttataggattcgatt
ttgatgtatactctgttttaactagtagaggcggtatcaccgaggcgaccttgtactttgaaaatgttactgaagaatatatag
gtaatacatataaatgtcgtggacacaactattattttgaaaaaacccttacaactacagtagtattggagtaaatacacaatg
catttttatatacattactgaataattattattattatttatatcgtatttgtgctataacgcgactatctaggtatttgtatc
tcaccgatagagaacatataaatgtagactctattaaacagttgtgtaaaatatcagatcctaatagatgtggatgtacggctt
tagaaatgagttcattaaaatatgtgatatcaacggaacatatttatataattatactattgctgttagtataattattgattc
cacggaagaactaccaacagttactccaattacaacaacatataattatactatcgatgatagcactactgaagaactacaagt
gactcctcatatggatctccatcgatgatacatgtattaaaatactttccgaataagtcttttaaatattgtattaattatgaa
aaactatgctatgcgagtatgatacgatactagattttatctctagcgagagatgtcgttagaatcatttatcaacgaatatcg
ataacatgtgtcatttatacgttaaagtctgtccgtcttctctattgtttagactgtttgtagaatgctgtgatataaacaaac
tagtagacacaaatatttaactcatgatgaagttgagaatgatatgctttagctaatataaaaatatattaatccactatatat
tctagacttgatttaaaaccgataaactactactacgtactgtataagttaggagcagaccctaattatgtagatgatagaggt
aatacttctgcatctatatgtccacttatgagaaaacgtcatttaataagatgcatcgtgaaaagaaatttattaaagagttgg
taaaatatgaaaccgaaagtaaataatataggaaatacacctctacataactacgtatctcaatatgatatcactctcattcct
catccacaacccattaaaaaatggaaattaaagccctctattagcataaacggctacaggtctacctttacaatggcctttcct
tgtgcccagttcagaccctgtcattgccacgctactaaggactccctgaataccgtggccgacgtcagacattgtctgactgaa
tacatcctgtgggtttctcatagatggacccatagagaaagcgcagggtctctctacaggcttctcatctctttcagaactgat
gcaacggagctctttggtggtgagttgaaggattcacttccgtggagatcattaaatgactccatgaaaaccgccgaagaactt
cgtgcaatcattggactttgtactcaatcagctatcgtctctggaagagtcttcaacgataagtatatcgacatactacttatg
ctgcgaaagattctgaacgagaacgactatctcaccctcttggatcatatccgcactgctaaatactaaatctccttcatgctc
tctcactacactttttatcatcttatgaggaataattagcaccagaatagctatggattgcacatgtattctatgtcgtctact
ggatgaagatgtgacgtacaaaaaaataaaactagaaattgaaacgtgtcacaacttatcaaaacatatagatagacgaggaaa
caatgcgctacattgttacgtctccaataaatgcgatacagacattaagattgttcgactgttactctctcgcggagtcgagag
actttgtagaaacaacgaaggattaactccgctaggagcatacagtaagcatagatacgtaaaatctcagattgtgcatctact
gatatccagctattcgaattcctctaacgaactcaagtcgaatataaatgatttcgacttacgtctgctaaaatacctaattgt
ggataaacggatacgtccgtccaagaatacgaattatgcaatcaatggtctcggattggtggatatatacgtaacgacgcctaa
tccgagaccagaagtattgctatggcttcttaaatcagaatgttacagcaccggttacgtatttcgtacctgtatgtacaacag
tgatatgtgtaagaactctcttcattactatatatcgtctcatagagaatctctatccaaggatgtaattaaatgtttgatcga
taacaatgtttccatccaatactactggtcttgctcaaccatagatatagagattattaataaaggatgtggacacgtgtagag
tatacgacgtcagccctatattagaggcgtattatctaaacaagcgatttagagtaaccccatataatgtagacatggaaatcg
ttaatcttcttattgagagacgtcatactcttgtcgacgtaatgcgtagtattacttcgtacgattccagagaatataaccact
acatcatcgataacattctaaagagatttagacaacaggatgaatccatcgtacaagccatactgataaactacttacattacg
gcgatatggtaagtatacctatcattcaatgcatgttggataagacgacggacaacaactttgttaataataatctcgtcgatg
taaacgtcgtaaggtttatcgtggaaaatatggacacgcggctgtaaatcacatatctaacaatggccgtctatgtatgtacgg
tctgatattatcgagatttaataattgcgggtatcactgttatgaagatgtatttgatatactaagcaagtacatggatgatat
agatatgatcgataactctactatattacgcggtcgatgtcaataatatacaatttgcaaagcggttattggaatatggagcga
gtgtcacgctcgataatcaatacggccatccagaaaagcagttaccaaagagaagctagttgatttattactgagttaccatcc
cactctagagactatgattgacgcatttaatagagatatacgctatctatatcctgaaccattattcgcctgtatcagatacgc
cttaatcctagatgatgattttccttctaaagtaagtatgatatcgccggtcgtcataaggaactaaagcgctatagagcagac
attaatagaatgaagaatgcctacatatcaggcgtctccatgtttgatatattatttaaacgaagcaaacgccacagattgaga
tacgcaaagaacaatgagaggatcgactccattaaataatttatcatggagtgataatgtcctgtttccatggcatattacaaa
atcgattccgtccaagatgataaaaacatttaccggcatcataaacacggagtttattttatatgtctcgcataaacattacta
aaaaaatatattgttcggttttctttcacatctttaattatgaaaaagtaaatcattatgagatggacgcatcgttcgcgacag
tatgtggtacatacctaacgtatttatggacgacggtaagaatgaaggtcacgtttctgtcaacaatgtcgacgcgatcgtgta
acacgactcacaatagaatctgtgaatgctctcccgatcatggatgcaaggcatgtgtttcccaaacaaaatgtggaataggat
acggagtatccggagacgtcatctgttctccgtgtggtctcggaacatattctcacaccgtctcttccgcagataaatgcgaac
ccgtacccagaaatacctttaactatatcgatgtggaaattaacctgtatccagttaacgacacatcgtgtactcggacgacca
ctaccggtctcagcgaatccatctcaacgtcggaactaactattactatgaatcataaagactgcgatcccgtcttcttaataa
ggtagcgacttcaggtttctttacaggagaaaggtgtgcactctgaatttcgagattaaatgcaataacaaagattcttcctcc
aaacagttaacgaaagcaaagaatgatactatcatgccgcattcggagacagtaactctagcgtcgacatctatatactatata
gtaataccaatactcaagactacgaaactgatacaatctcttatcatgtgggtaatgtagccatatgcccggtagttgcgatat
acataaactgatcactaattccaaacccacccgctttttatagtaagtttttcacccataaatacaataattaatttctcgtaa
aagtagaaaatatattctaatttattgcacggtaaggaagtagaatcataaagaacagtactcaatcaatagcaatcatgaaac
aatatatcgtactggcatgcatgtgcctgccagtcttcagcaatcatcctcatcgtgtacggaagaagaaaacaaacatcatat
gggaatcgatgttattatcaaagtcacaaagcaagaccaaacaccgaccgatgataagatttgccaatccgtaacggaaattac
agagtccgagtcagatccagatcccgaggtggaatcagtcgaggatgtagatcctcctaccacttattactccatcatcggtgg
aggtctgagaatgaactttggattcaccaaatgtcctcagattaaatccatctcagaatccgctgatggaaagactgtgaggtg
tctatcgacatcagatgtagcgaagaagagaaagacagcgacatcaagacccatccagtactcgggtctaacatctctcataag
aaagtgagttacgaagatatcatcggttcaacgatcgtcgatacaaaatgtgtcaagaatctagagtttagcgttcgtatcgga
gacatgtgcaaggaatcatctgaacttgaggtcaagtatgtcgacggatcggcatctgaaggtgcaaccgatgatacttcactc
atcgattcaacaaaactcaaagcgtgtgtctgaatcgataactctattcatctgaaattggatgagtagggttaatcgaacgat
tcaggcacaccacgaattaaaaaagtgtaccggacactatattccggtttgcaaaacaaaaagttacctctcgcgacttcttct
ttttctgtctcaatagtgtgatacgattatgacactattcctatttcctttcagggtatcacaaaaatattaaacctctttctg
atggtctcatacaaaaatatttttattctctttctctctttgatggtctcataaaaaatatttttattctctttctctctttga
tggtctcataaaatatttttattctctttctctctttgatggtctcataaaaaatatttttattctctttctctctttgatggt
ctcataaaatatttttattctctttctctctttgatggtctcataaaaaatatttttattctctttctctctttgatggtctca
taaaaaatattaaacctctttctgatggtgtcactaaaatatttttattctctttctctcttcaatggagtcataaaatatttt
tattctctttctctcttcgatggtctcacaaaaatattaaacctctttctgatggtgtcactaaaatatttttattctctttct
ctcttcaatggagtcataaaatatttttattctctttctctctttgatggtctcacaaaaatatttttattctctttctctctt
tgatggtctcacaaaaatatttttattctctttctctctttgatggtctcacaaaaatatttttattctctttctctctttgat
ggtctcataaaaaaagttttacaaaaatatttttattctctttctctctttgatggtctcataaaaaaagttttacaaaaatat
ttttattctctttctctctttgatggtctcataaaaaaagttttacaaaaatatttttattctctttctctctttgatggtctc
ataaaaaatattaaacctctttctgatggtgtcactaaaatatttttattctcattctctcttcaatggagtcataaaatattt
ttattctctttctctcttcgatggtctcacaaaaatattaaacctctttctgatggtgtcactaaaatatttttattctcattc
tctcttcaatggagtcataaaatatttttattctctttctctcttcgatggtctcacaaaaatattaaacctctttctgatggt
gtcactaaaatatttttattctcattctctcttcaatggagtcataaaatatttttattctctttctctctttgatggtctcat
aaaaaaagttttacaaaaatatttttattctctttctctctttgatggtctcataaaaaatattaaacctctttctgatggtgt
cactaaaatatttttattctctttctctcttcaatggagtcataaaatatttttattctctttctctcttcgatggtctcacaa
aaatattaaacctctttctgatggtgtcactaaaatatttttattctcattctctcttcaatggagtcataaaatatttttatt
ctctttctctctttgatggtctcataaaaaaagttttacaaaaatatttttattctctttctctctttgatggtctcataaaaa
aagttttacaaaaatatttttattctctttctctctttgatggtctcataaaaaaagttttacaaaaatatttttattctcttt
ctctctttgatggtctcataaaaaaagttttacaaaaatatttttattctctttctctctttgatggtctcataaaaaatatta
aacctctttctgatggtgtcactaaaatatttttattctcattctctcttcaatggagtcataaaatatttttattctctttct
ctcttcgatggtctcacaaaaatattaaacctctttctgatggtgtcactaaaatatttttattctcattctctcttcaatgga
gtcataaaatatttttattctctttctctctttgatggtctcataaaaaaagttttacaaaaatatttttattctctttctctc
tttgatggtctcataaaaaaagttttacaaaaatatttttattctctttctctctttgatggtctcataaaaaaagttttacaa
aaatatttttattctctttctctctttgatggtctcataaaaaaagttttacaaaaatatttttattctctttctctctttgat
ggtctcacaaaaatattaaacctctttctgatggagtcgtaaaaaagttttatctctttctccttcgatggtctcacaaaaata
ttaaacctctttctgatggagtcgtaaaaaagttttatctctttctctcttcgatggtctcacaaaaatattaaacctctttct
gatggagtcgtaaaaaagttttatctctttctctcttcgatggtctcactaaaatattttttattctctttctgatgcatcaac
tatttcttaaacaataacgtccaacaacatatactcgtcgagcttatcaacatcccctatgcccatctaggttaccagacaatt
gtatatcataaaataatgtttataatttttacaaaaatatttttattctctttctctctttgatggtctcataaaaaaagtttt
acaaaaatatttttattctctttctctctttgatggtctcataaaaaatattaaacctctttctgatggtgtcactaaaatatt
tttattctcattctctcttcaatggagtcataaaatatttttattctctttctctcttcgatggtctcacaaaaatattaaacc
tctttctgatggtgtcactaaaatatttttattctcattctctcttcaatggagtcataaaatatttttattctctttctctct
ttgatggtctcataaaaaaagttttacaaaaatatttttattctctttctctctttgatggtctcataaaaaaagttttacaaa
aatatttttattctctttctctctttgatggtctcataaaaaaagttttacaaaaatatttttattctctttctctctttgatg
gtctcataaaaaaagttttacaaaaatatttttattctctttctctctttgatggtctcataaaaaaagttttacaaaaatatt
tttattctctttctctctttgatggtctcataaaaaaagttttacaaaaatatttttattctctttctctctttgatggtctca
taaaaaaagttttacaaaaatatttttattctctttctctctttgatggtctcataaaaaatattaaacctctttctgatggtg
tcactaaaatatttttattctctttctctcttcaatggagtcataaaatatttttattctctttctctcttcgatggtctcaca
aaaatattaaacctctttctgatggtgtcactaaaatatttttattctcattctctcttcaatggagtcataaaatatttttat
tctctttctctctttgatggtctcataaaaaaagttttacaaaaatatttttattctctttctctctttgatggtctcataaaa
aaagttttacaaaaatatttttattctctttctctctttgatggtctcataaaaaaagttttacaaaaatatttttattctctt
tctctctttgatggtctcataaaaaaagttttacaaaaatatttttattctctttctctctttgatggtctcataaaaaaagtt
ttacaaaaatatttttattctctttctctctttgatggtctcataaaaaatattaaacctctttctgatggtgtcactaaaata
tttttattctctttctctcttcaatggagtcataaaatatttttattctctttctctcttcgatggtctcacaaaaatattaaa
cctctttctgatggtgtcactaaaatatttttattctctttctctcttcaatggagtcataaaatatttttattctctttctct
ctttgatggtctcataaaaaaagttttacaaaaatatttttattctctttctctctttgatggtctcataaaaaaagttttaca
aaaatatttttattctctttctctctttgatggtctcataaaaaaagttttacaaaaatatttttattctctttctctctttga
tggtctcataaaaaaagttttacaaaaatatttttattctctttctctctttgatggtctcataaaaaaagttttacaaaaata
tttttattctctttctctctttgatggtctcataaaaaaagttttacaaaaatatttttattctctttctctctttgatggtct
cataaaaaaagttttacaaaaatatttttattctctttctctctttgatggtctcataaaaaatattaaacctctttctgatgg
tgtcactaaaatatttttattctcattttctctttctctcttcaatggagtcataaaatatttttattctctttctctctttga
tggtctcataaaaaatattaaacctctttctgatggtgtcactaaaatatttttattctcattctctcttcaatggagtcataa
aaaagttttatctctttctctcttcgatggtctcacaaaaatattaaacctctttctgatggagtcgtaaaaaagttttatctc
tttctctcttcgatggtctcacaaaaatattaaacctctttctgatgcatcaactatttcttaaacaataacgtccaacaacat
atactcatcccctatgcccatctaggttaccagacaattgtatatcataaaataatgtttataatttacacgttaaaatcatat
aataaaacgtagatcgtataatattttttggtatataaatgatctagtaaaatccatgtaggggatactgctcacattttttct
ttggtacaaaatttcacacaagtttttatacagacaaattcttgtccatatattttaaaacattgacttttgtactaagaaaaa
tatctagactaactatctctttctctttctctcttcgatggtctttctgatggagtcgtaaaaaagttttatctctttctctct
tcgatggtctcacaaaaatattaaacctctttctgatggagtcgtaaaaaagttttatctctttctctcttcgatggtctcaca
aaaatattaaacctctttctgatggagtcgtaaaaaagttttatctctttctccttcgatggtctcacaaaaatattaaacctc
tttctgatggagtcgtaaaaaagttttatctctttctctcttcgatggtctcacaaaaatattaaacctctttctgatggtgtc
actaaaatatttttattctctttctctcttcgatggtctcacaaaaatattaaacctctttctgatggagtcgtaaaaaagttt
tatctctttctccttcgatggtctcacaaaaatattaaacctctttctgatggagtcgtaaaaaagttttatctctttctctct
tcgatggtctcacaaaaatattaaacctctttctgatggagtcgtaaaaaagttttatctctttctccttcgatggtctcacaa
aaatattaaacctctttctgatggagtcgtaaaaaagttttatctctttctccttcgatggtctcacaaaaatattaaacctct
ttctgatggagtcgtaaaaaagttttatctctttctccttcgatggtctcacaaaaatattaaacctctttctgatggagtcgt
aaaaaagttttatctctttctctcttcgatggtctcacaaaaatattaaacctctttctgatggagtcgtaaaaaagttttatc
tctttctctcttcgatggtctcacaaaaatattaaacctctttctgatggagtcgtaaaaaagttttatctctttctccttcga
tggtctcacaaaaatattaaacctctttctgatagtctctataaagcgattgatttttcttaccctctagagtttcctacggtc
gttggtcacacatttttttctagacactaaataaatagtaagattaaattaattataaaattatgtatataatattaattataa
aattatgtatatgatttactaactttagttagataaattaataatacataaattttagtatattaatattataaattaataata
cataaattttagtatattaatattatattttaaatatttatttagtgtctagaaaaaaaggccggcctagcaggcc

Also disclosed are examples of synthetic Vaccinia virus (sVAC) fragments to reconstitute the complete Vaccinia virus genome. An example sequence of sVAC fragment 1 (F1, 66679 bp in length) (SEQ ID NO: 16):

tttttttctagacactaaataaaatatttaaaatataatattaatgtactaaaacttatatattattaatttatctaactaaagtta
gtaaattatatatataattttataattaatttaattttactaattttatttagtgtctagaaaaaaatgtgtgacccatgactgtag
gaaactctagagtgtaagaaagatcgatcgctttatagagaccatcagaaagaggtttaatatttttgtgagaccatcgaagagaga
aagagataaaacttttttacgactccatcagaaagaggtttaatatttttgtgagaccatcgaagagagaaagagataaaacttttt
tacgactccatcagaaagaggtttaatatttttgtgagaccatcgaaggagaaagagataaaacttttttacgactccatcagaaag
aggtttaatatttttgtgagaccatcgaaggagaaagagataaaacttttttacgactccatcagaaagaggtttaatatttttgtg
agaccatcgaagagagaaagagataaaacttttttacgactccatcagaaagaggtttaatatttttgtgagaccatcgaagagaga
aagagataaaacttttttacgactccatcagaaagaggtttaatatttttgtgagaccatcgaaggagaaagagataaaactttttt
acgactccatcagaaagaggtttaatatttttgtgagaccatcgaagagagaaagagataaaacttttttacgactccatcagaaag
aggtttaatatttttgtgagaccatcgaagagagaaagagataaaacttttttacgactccatcagaaagaggtttaatatttttgt
gagaccatcgaagagagaaagagataaaacttttttacgactccatcagaaagaggtttaatatttttgtgagaccatcgaagagag
aaagagataaaacttttttacgactccatcagaaagaggtttaatatttttgtgagaccatcgaagagagaaagagataaaactttt
ttacgactccatcagaaagaggtttaatatttttgtgagaccatcgaagagagaaagagaaagagatagttgatctagatatttttc
ttagtacaaaagtcaatgttttaaaatatatggacaagaatttgtctgtataaaaacttgtgtgaaattttgtaccaaagaaaaaat
gtgagcagtatcccctacatggattttactagatcatttatataccaaaaaatattatacgatctacgttttattatatgattttaa
cgtgtaaattataaacattattttatgatatacaattgtctggtaacctagatgggcataggggatgttgataagctcgacgagtat
atgttgttggacgttattgtttaagaaatagttgatgcatcagaaagagaataaaaaatattttagtgagaccatcgaagagagaaa
gagataaaacttttttacgactccatcagaaagaggtttaatatttttgtgagaccatcgaagagagaaagagataaaactttttta
cgactccatcagaaagaggtttaatatttttgtgagaccatcgaagagagaaagagataaaacttttttacgactccatcagaaaga
ggtttaatatttttgtgagaccatcgaagagagaaagagataaaacttttttacgactccatcagaaagaggtttaatatttttgtg
agaccatcgaagagagaaagagataaaacttttttacgactccatcagaaagaggtttaatatttttgtgagaccatcgaagagaga
aagagataaaacttttttacgactccatcagaaagaggtttaatatttttgtgagaccatcgaagagagaaagagataaaacttttt
tacgactccatcagaaagaggtttaatatttttgtgagaccatcgaagagagaaagagataaaacttttttacgactccatcagaaa
gaggtttaatatttttgtgagaccatcgaagagagaaagagataaaacttttttacgactccatcagaaagaggtttaatatttttg
tgagaccatcgaagagagaaagagataaaacttttttacgactccatcagaaagaggtttaatatttttgtgagaccatcgaagaga
gaaagagataaaacttttttacgactccatcagaaagaggtttaatatttttgtgagaccatcgaagagagaaagagataaaacttt
tttacgactccatcagaaagaggtttaatatttttgtgagaccatcgaagagagaaagagataaaacttttttacgactccatcaga
aagaggtttaatatttttgtgagaccatcgaagagagaaagagataaaacttttttacgactccatcagaaagaggtttaatatttt
tgtgagaccatcgaagagagaaagagataaaacttttttacgactccatcagaaagaggtttaatatttttgtgagaccatcgaaga
gagaaagagataaaacttttttacgactccatcagaaagaggtttaatatttttgtgagaccatcgaagagagaaagagataaaact
tttttacgactccatcagaaagaggtttaatatttttgtgagaccatcgaagagagaaagagataaaacttttttacgactccatca
gaaagaggtttaatatttttgtgagaccatcgaagagagaaagagaataaaaatattttatgactccattgaagagagaaagagaaa
atgagaatgagaataaaaatattttagtgacaccatcagaaagaggtttaatatttttgtgagaccatcgaagagagaaagagaata
aaaatattttatgactccattgaagagagaaagagaaaatgagaatgagaataaaaatattttagtgacaccatcagaaagaggttt
aatattttttatgagaccatcaaagagagaaagagaataaaaatatttttgtaaaactttttttatgagaccatcaaagagagaaag
agaataaaaatatttttgtaaaactttttttatgagaccatcaaagagagaaagagaataaaaatatttttgtaaaactttttttat
gagaccatcaaagagagaaagagaataaaaatatttttgtaaaactttttttatgagaccatcaaagagagaaagagaataaaaata
tttttgtaaaactttttttatgagaccatcaaagagagaaagagaataaaaatatttttgtaaaactttttttatgagaccatcaaa
gagagaaagagaataaaaatatttttgtaaaactttttttatgagaccatcaaagagagaaagagaataaaaatatttttgtaaaac
tttttttatgagaccatcagaaagaggtttaatatttttgtgataccctgaaaggaaataggaataggaataggaatagtgtcataa
tcgtatcacactattgagacagaaaaagaagaagtcgcgagaggtaactttttgtgaatgtagttaagaacatttttgttttgcaaa
ccggaatatagtgtccggtacacttttttaattcgtggtgtgcctgaatcgttcgattaaccctactcatccaatttcagatgaata
gagttatcgattcagacacacgctttgagttttgttgaatcgatgagtgaagtatcatcggttgcaccttcagatgccgatccgtcg
acatacttaaatccatccttgacctcaagttcagatgattccttgcacatgtctccgatacgaacgctaaactctagattcttgaca
cattttgtatcgacgatcgttgaaccgatgatatcttcgtaactcactttcttatgagagatgttagacccgagtactggatgggtc
ttgatgtcgctgtctttctcttcttcgctacatctgatgtcgatagacacctcacagtctttgatcatagccagagcttcttcatga
gtgatcgcgggagagtccttaccttgtcctggggacacgctggacaatctagcattcactgtgtttccatcagcggattctgagatg
gatttaatctgaggacatttggtgaatccaaagttcattctcagacctccaccgatgatggagtaataagtggtaggaggatctaca
tcctcgactgatgtggaatcatcttctgattccacctcgggatctggatctgactcggactctgtaatttccgttacggattggcaa
atcttatcattggtcggtgtttggtcttgctttgtgactttgataataacatcgattcccatatgatgtttgttttcttcttccgta
cacgaggaggaggatgaggatgattgctgaagactggcaggcacatgcatgccaggacgatatattgtttcatgattgctattgatt
gagtactgttctttatgattctacttccttaccgtgcaataaattagaatatattttctacttttacgagaaattaattattgtatt
tattatttatgggtgaaaaacttactataaaaagcgggtgggtttggaattagtgatcagtttatgtatatcgcaactaccgggcat
atggctatcgacatcgagaacattacccacatgataagagattgtatcagtttcgtagtcttgagtattggtattactatatagtat
atagatgtcgacgctagatagacagtctccgaatgcggcatgataccgtcatcattctttgctttcgttaactgtttggaggaaaaa
tttttgttattgcatttaatctcgaaattcagagtgcacacctttctcctgtaaagaaacctgaagttgctaccttattaaggacgg
agaagtattcctcacgaaatacgggattacagtctttatgattcatagtaatagttagttccgacgttgagatggattcgctgagac
cggtagtggtcgttaactggatacagattaatttccacatcgatatagttaaaggtattactgggtacgggttcgcatttatctgcg
gaagagacggtgtgagaatatgttccgagaccacacggagaacagatgacgtctccggatactccgtatcctattccacattttgtt
tgggaaacacatgccttgcatccggatgatcctttgagaagacaataatatccgggagagcattcacagattctattgtgagtcgtg
ttacacggtcgcgtcttccgttacaacttagacaagcgggtaaatgattattgcgagatgtgaaggtacccgaaccacacggcgtac
attgtgtgttagtcttgctatcgcataatctggaagcgtatgttcccggacacaaattatggcgtttgtattcgttgtctttacact
ttccatcggatggtgcatgcggtgctatatctcttccgtttattattatacatgagagaaacaatatatacgagtataatacggact
tcatgatttaataatgtagtaatcgtcgtcttgttcctgtttcctacttctccaatcatatagatattttctttctatcatggataa
tatttgtaatggttcttttcgtacaacatactgtttagatgatattgcgcataatttccggaggcaaatacgatagtctagattgac
cgatggtagactctaatttattgagtgctttgtcgacgagtttacttttacgctccatcgatagatggcactgttctatgagatcgt
cgtacatgggaaatgaaatgtgactgtctgaatgtatggctttaagatagctgtgataccgtatacaggtcggtgtcggagattcga
atctctttaaggcgacttatgtcacgatgatggaatctatcttatcgaatgatatatttttcataaatacacttttatagtcctcgt
ttaaacagaatttactatgtagttccgcgaatgactcgtcccttaataggcagtaggctattatcttctttacgtagtaatcgtcgt
agggagagacatcttgtagaacaacgatttaatcataggtagagatactttcagtctgtggtggatgatgtcattcacaacatccgc
cttgtatatgatgtttctgttttcaaacaccaagtcgaataccgtctttagtcggaaggttgatgtcgtatccgatgtatgaggcaa
cattgttgttacaattttgaaaggcggtattatagtattcgtctttctgaatgtcgaacctatctagtagataccgtagtatattga
gagtgtatccttgattatgttttatgaatagataaagtagatgttgtccttcttccttttgttcgtgccaattgagtaacattatga
gaatatgacctgttgcacaatcgttccatgatgggtgtacaatcaagattattacgtatcctcgtatcggctcctcgagataaaaga
gcatacaccacacgaggactatgtttggtatactgttgaaggtaagtgtgtaaccgcgttaatgtttgctccataatctattatcgc
gtagatgaatcgcttctcggctcgcatcttagtgtgacttaacttgtaataattgcttttgtagaacgtggatatgtgtttacagta
gtaatgaagagaagtgagttcatcctcgtcggatcctttgtacagaacgtaatagtttaagctcccattgaatttatatctaagata
acacagcaatagatcggatgatttactaaagtcatcaatggtgtccgttagtatatcaaagatcttgttatcgattgatagtggtgt
cctttttcatccttgctatcaaagttacgcatgccgtggtgtaacaatatctttaatacagatggattaaatcgtgtattcatcgta
tagcaatgtaatggagagttacctcgtttattcagatcgcagtgtttaataactagcttaaacagatgagacgatgtatccacatca
aagaacgtgaaatacatatgacagacattgttgacagaaacgtgaccttcattcttaccgtcgtccataaatacgttaggtatgtac
cacatactgtcgcgaacgatgcgtacaatctcgtccatctcataatgatttactttttcataattaaagatgtgaaagaaaaacaga
acaatatatttttttagtaatgtttatgcgagacatataaaataaactccgtgtttatgatcatttttaacagcaacacattcaata
ttgtattgttatttttatattatttacacaattaacaatatattattagtttatattactgaattaataatataaaattcccaatct
tgtcataaacacacactgagaaacagcataaacacaaaatccatcaaaaatgtcgatgaaatatctgatgttgttgttcgctgctat
gataatcagatcattcgccgatagtggtaacgctatcgaaacgacatcgccagaaattacaaacgctacaacagatattccagctat
cagattatgcggtccagagggagatggatattgtttacacggtgactgtatccacgctagagatattgacggtatgtattgtagatg
ctctcatggttatacaggcattagatgtcagcatgtagtattagtagactatcaacgttcagaaaacccaaacactacaacgtcata
tatcccatctcccggtattatgcttgtattagtaggcattattattattacgtgttgtctattatctgtttataggttcactcgacg
aactaaactacctatacaagatatggttgtgccataatttttataaatttttttatgagtatttttacaaaaaaaatgtataaagtg
tatgtcttatgtatatttataaaaatgctaagtatgcgatgtatctatgttatttgtatttatctaaacaatacctctacctctaga
tattatacaaaaattttttatttcggcatattaaagtaaaatctagttaccttgaaaatgaatacagtgggtggttccgtatcacca
gtaagaacataatagtcgaatacagtatccgattgagattttgcatacaatactagtctagaaagaaatttgtaatcatcttctgtg
acgggagtccatatatctgtatcatcgtctagtttatcagtgtcccatgctatattcctgttatcatcattagttaatgaaaataac
tctcgtgcttcagaaaagtcaaatattgtatccatacatacatctccaaaactatcgcttatacgtttatctttaacgatacctata
cctagatggttatttactaacagacattttccagatctattgactataactcctatagtttccacatcaaccaagtaatgatcatct
attgttatataacaataacataactcttttccatttttatcagtatgtatatctatatcaacgtcgtcgttgtagtgaatagtagtc
attgatctattatatgaaacggatatgtctagaacggcaattgttttacgtccagttaacactttctttgatttaaagtctagagtc
tttgcaaacataatatccttatccgactttatatttcctgtagggtggtataattttattttgcctccacatatcggtgtttccaaa
tatattactagacaatattccatatagttattagttaagggtacccaattagaacacgtacgcttattatcatcatttggatcgtat
ttcataaaagttattgtactatcgatgtcaacacattctacattttttaatcgtctatatagtatttttctgatattttctataata
tcagaattgtcttccatcggaagttgtatactatcggaatcagttacatgtttaaataattctctgatgtcattccttatacaatca
aattcattattaaacagtttaatagtctgtagacctttatcgtcgtaaatatccattgtcttattagttacgcttatttttatgtgt
tttacgttgctttattatattttataagaatgattgtttgacgaatcacgagaactattaagacacattattaggtatatattataa
aaaagtttttgattacgatgttataagaggaaagaggacacattaacatcatacatcaattaactacattcttataacatcgtaatc
aaaagaattgcaattttgatgtataacaactgtcaatgggttatggaattgtatattacatattatacggtatgttggtaacgacaa
ataccgatcggtaattgtctgccggtgtaatagaattatatatatctatctattacaccggctgagtatgcataataataagttgtg
gtagtatgatctccatatttataatttaggactttgtattcagtatttttggaatcataaaaaataaaaaaaagttttactaattta
aaatttaaaaagtatttacatttttttcactgtttagtcgcggatatggaattcgatcctgccaaaatcaatacatcatctatagat
catgtaacaatattacaatacatagatgaaccaaatgatataagactaacagtatgcattatccgaaatattaataacattacatat
tatatcaatatcacaaaaataaatacacatttggctaatcaatttcgggcttggaaaaaacgtatcgccggaagggactatatgact
aacttatctagagatacaggaatacaacaatcaaaacttactgaaactatacgtaactgtcaaaaaaatagaaacatatatggtcta
tatatacactacaatttagttattaatgtggttattgattggataaccgatgtgattgttcaatcaatattaagagggttggtaaat
tggtacatagctaataatacctatacacccaataatacaacaaccatttctgagttggatatcatcaaaatactggataaatacgag
gacgtgtatagagtaagtaaagaaaaagaatgtggaatttgctatgaagttgtttactcaaaacgatagatactttggtttattgga
ttcgtgtactcatatattttgcataacatgcatcaatatatggcataaaacacgaagagaaaccggtgcgtcggataattgtcctat
atgtcgtacccgttttagaaacataacaatgagcaagttctataagctagttaactaataaataaaaagtttaatttgttgacgacg
tatgtcgttatttttctcgtatgaaagattaaattcaattcaattcgttgtttctaatataatctgccgtattggatggattctcaa
gacaattgcatttagattatattatcatgaataaaaatagtagcacgcactacttcagccaaatattcttttttgaaacgccatcta
tcgtagtgaggacacaagtgaacctataattatcaaatttattagtatcagtcacatgaaggactttctgtagagtgacgattctac
catctatggtactaacggtttcatcctccttgataccctcacccaaatgttctataaatttagcatcctcgtccgatctcatatcct
ttgccaaccaatacatgtagctaaaattaggcataaatttcacacatccagtgcaacgaaattctccagaagatgttacgatgttta
ggttaggacatttgatttcgtcggcattaacatatgggtgaacacacccatacatgaaagcgatgagaaataggattctcatcttgc
caaaatatcactagaaaaaatttatttatcaattttaaaggtataaaaaatacttattgttgctcgaatattttgtatttgatggta
tacggaagattagaaatgtaggtattatcatcaactgattctatggttttatgtattctatcatgtttcactattgcgtcggaaata
atatcatatgcttccacatatattttattttgttttaactcataatactcacgtaattctggattattggcatatctatgaataatt
ttagctccatgatcagtaaatattaatgagaacatagtattaccacctaccattatttttttcatttcgttcaattcttgattgcaa
agatctatataatcattatagcgttgacttatggactctggaatcttagacgatgtacagtcatctataatcatggcatatttaata
cattgttttatagcatagtagttatctacgatgttagatatttctctcaatgaatcaatcacacaatctaatgtaggtttatgacat
aatagcattttcagcagttcaatgtttctagattcgttgatggcaatggctatacatgtatatccgttatttgatctaatgttgaca
tctgaaccggattctagcagtaaagatactagagattgtttattatatctaacagccttgtgaagaagtgtttctcctcgtttgtca
atcatgttaatgtctttaagataaggtaggcaaatgtttatagtactaagaattgggcaagcataagacatgtcacaaagacccttt
ttgtatgtataagtgtaaaaattataacattcatagttggatttacataggtgtccaatcgggatctctccatcatcgagataattg
atggcatctcccttccttttttagtagatatttcatcgtgtaagaatcaatattaatatttctaaagtattcgtgtatagcctcttt
atttaccacagttccatattccactagagggatatcgccgaatgtcatatactcaattagtatatgttggaggacatccgagttcat
tgttttcaatatcaaaaagatggtttccttatcatttctccatagtggtacaatactacacattattccgtgcggctttccattttc
caaaaacaatttgaccaaatctaaatctacatctttattgtatctataatcactatttagataatcagccataattactcgagtgca
acatgttagatcgtctatatatgaataagcagtgttatctattcctttcattaacaatttaacgatgtctatatctatatgagatga
cttaatataatattgaagagctgtacaatagtttttatctatagaagacggcttgattccgtgattaattagacatttaacaacttc
cggacgcacatatgctctcgtatccgactttgaatacagatgagagatgatatacagatgcaatacggtaccgcaatttcgtagttg
ataatcatcatacgcgtatcagtactcgtcctcataaagaacactgcagccattttctatgaacaaatcaataattttaggaacagg
atcattgtcattacataattttctataactgaacgatggttttcacatttaacactcaagtcaaatccatgttctaccaacaccttt
atcaagtcaacgtctacatttttggatttcatatagctgaatatattaaagtcatttatgttgctaaatccagtggcttctagtaga
gccatcgctatatcctttaactttaacatgtctactatttgtgtattcttctaatggggtagctgtctccaatttttgcgtaatgga
ttagtgccactgtctagtagtagtttgacgacctcgacattattacaatgctcattaaaaaggtatgcgtgtaaagcattattcttg
aattggttcctggtatcattaggatctctgtctctcaacatctgtttaagttcatcgagagccacctcctcattttccagatagtca
aacattttgactgaatgagctactgtgaactctatacacccacacaactaatgtcattaaatattatttttttgaatgtatttatac
catgtcaaaaacttgtacaattattaataaaaataatttagtgtttaaattttaccagttccagattttacacctccgttaacccca
ctttttacaccactggacgatcctcctccccacattccaccgccaccagatgtataagttttagatcctttattactaccatcatgt
ccatggataaagacactccacatgccgccactactaccccctttagaagacatattaataagacttaaggacaagtttaacaataaa
attaatcacgagtaccctactaccaacctacactattatatgattatagtttctatttttacagtaccttaactaaagtctctagtc
acaagagcaatactaccaacctacactattatatgattatagtttctatttttataggaacgcgtacgagaaaatcaaatgtctaat
ttctaacggtagtgttgataaacgattatcgtcaatggatacctcctctatcatgtcgtctattttcttactttgttctattaactt
attagcattatatattatttgattataaaacttatattgcttattagcccaatctgtaaatatcggattattaacatatcgtttctt
tgtaggtttatttaacatgtacatcactgtaagcatgtccgtaccatttattttaatttgacgcatatccgcaatttctttttcgca
gtcggttataaattctatatatgatggatacatgctacatgtgtacttataatcgactaatatgaagtacttgatacatattttcag
taacgatttattattaccacctatgaataagtacctgtgatcgtctaggtaatcaactgttttcttaatacattcgatggttggtaa
tttactcagaataatttccaatatcttaatatataattctgctatttctgggatatatttatctgccagtataacacaaatagtaat
acatgtaaacccatattttgttattatattaatgtctgcgccattatctattaaccattctactaggctgacactatgcgacttaat
acaatgataaagtatactacatccatgtttatctattttgtttatatcatcaatatacggcttacaaagttttagtatcgataacac
atccaactcacgcatagagaaggtagggaataatggcataatatttattaggttatcatcattgtcattatctacaactaagtttcc
attttttaaaatatactcgacaactttaggatctctattgccaaatttttgaaaatatttatttatatgcttaaatctatataatgt
agctccttcatcaatcatacatttaataacattgatgtatactgtatgataagatacatattctaacaatagatcttgtatagaatc
tgtatatcttttaagaattgtggatattaggatattattacgtaaactattacacaattctaaaatataaaacgtatcacggtcgaa
taatagttgatcaactatataattatcgattttgtgatttttcttcctaaactgtttacgtaaatagttagatagaatattcattag
ttcatgaccactatagttactatcgaataacgcgtcaaatatttcccgtttaatatcgcatttgtcaagataataatagagtgtggt
atgttcacgataagtataataacgcatctcttttttgtgtgaaattaaatagtttatcacgtccaaagatgtagcataaccatcttg
tgacctagtaataatataataatagagaactgttttacccattctatcatcataatcagtggtgtagtcgtaatcgtaatcgtctaa
ttcatcatcccaattataatattcaccagcacgtctaatctgttctattttgatcttgtatccatactgtatgttgctacatgtagg
tattcctttatccaataatagtttaaacacatctacattgggatttgatgttgtagcgtatttctctacaatattaataccattttt
gatactatttatttctatacctttcgaaattagtaatttcaataagtctatatcgatgttatcagaacatagatattcgaatatatc
aaaatcattgatatttttatagtcgactgacgacaataacaaaatcacaacatcgtttttgatattattatttttcttggtaacgta
tgcctttaatggagtttcaccatcatactcatataatggatttgcaccactttctatcaatgattgtgcactgctggcatcgatgtt
aaatgttttacaactatcatagagtatcttatcgttaaccatgattggttgttgatgctatcgcattttttggtttctttcatttca
gttatgtatggatttagcacgtttgggaagcatgagctcatatgatttcagtactgtagtgtcagtactattagtttcgatcagatc
aatgtctagatctatagaatcaaaacacgataggtcagaagataatgaatatctgtacgcttctttttgtactgtaacttctggttt
tgttagatggttgcatcgtgctttaacatcaatggtacaaattttatcctcgctttgtgtatcatattcgtctctagtataaaattc
tatattcagattatcatgcgatgtgtatacgctaacggtatcaataaacggagcacaccatttagtcataacagtaatccaaaattt
tttaaagtatatcttaacgaaagaagttgtgtcattgtctacggtgtatggtactagatcctcataagtgtatatatctagagtaat
gtttaatttattaaatggttgataatatggatcctcatgacaatttccgaagatggaaatgagatatagacatgcaataaatctaat
cgaagacatggttactccttaaaaaaatacgaataatcaccttggctatttagtaagtgtcatttaacactatactcatattaatcc
atggactcataatctctatacgggattaacggatgttctatatacggggatgagtagttttcttctttaactttatactttttacta
atcatatttagactgatgtatgggtaatagtgtttaaagagttcgttctcatcatcagaataaatcaatatctctgtttttttgtta
tacagatgtattacagcctcatatattacgtaatagaacgtgtcatctaccttattaactttcaccgcatagttgtttgcaaatacg
gttaatcctttgacctcgtcgatttccgaccaatctgggcgtataatgaatctaaactttaatttcttgtaatcattcgaaataatt
tttagtttgcatccgtagttatcccctttatgtaactgtaaatttctcaacgcgatatctccattaataatgatgtcgaattcgtgc
tgtatacccatactgaatggatgaactaacgaatatcaacggcgttaatagtaatttactttttcatctttacatattgggtactag
ttttactatcataagtttataaattccacaagctactatggaataagccaaccatcttagtataccacacatgtcttaaagtttatt
aattaattacatgttgttttatatatatcgctacgaatttaaagagaaatcagtttaggaagaaaaaaattatctatctacatcatc
acgtctctgtattctacgatagagtgctactttaagatgagacatatccgtgtcatcaaaaatatactccattaaaatgattattcc
ggcagcgaacttgatattggatatatcacaacctttgttaatatctacgacaatagacagcagtcccatggttccataaacagtgag
tttatctttctttgaagcgatagtttgtagagatcttataaaaccgtcaaacgacatcgcatttatatctttagctaattcatatat
gttaccatcgtaatatctaaccgcgtctatcttaaacgtttccatcgctttaaagacgtttccgatagatggtctcatttcatcagt
catactgagccaacaaatataatcgtgtataacatctttgatagaatcagactctaaagaaaacgaatcggctttattatacgcatt
catgataaacttaatgaaaaatgtttttcgttgtttaagttggatgaatagtatgtcttaataattgttattatttcattaattaat
atttagtaacgagtacactctataaaaacgagaatgacataactagttatcaaagtgtctaggacgcgtaattttcatatggtatag
atcctgtaagcattgtctgtattctggagctattttctttatcgcattagtaagttcagaatatgttataaatttaaatcgaataac
gaacatgactttagtaaagtcgtctatattaactcttttattttctagccatcgtaataccatgtttaagatagtatattctctagt
tactacgatctcatcgttgtctagaatatcacatactgaatctacatccaattttagaaattggtctgtgttacatatctcttctat
attattgttgatgtattgtcgtagaaaactattacgtagaccattttctttataaaacgaatatatagtactccaattatctttacc
gatatatttgcacacataatccattctctcaatcactacatctttaagattttcgttgttaagatatttggctaaactatataattc
tattagatcatcaacagaatcagtatatatttttctagatccaaagacgaactctttggcgtcctctataatattcccagaaaagat
attttcgtgttttagtttatcgagatctgatctgttcatatacgccatgattgtacggtacgttatgataaccgcataaaataaaaa
tccattttcatttttaaccaatactattcataattgagattgatgtaatactttgttactttgaacgtaaagacagtacacggatcc
gtatctccaacaagcacgtagtaatcaaatttggtgttgttaaacttcgcaatattcatcaatttagatagaaacttatactcatca
tctgttttaggaatccatgtattattaccactttccaacttatcattatcccaggctatgtttcgtccatcatcgttgcgcagagtg
aataattcttttgtattcggtagttcaaatatatgatccatgcatagatcggcaaagctattgtagatgtgatttttcctaaatcta
atataaaactcgtttactagcaaacactttcctgatttatcgaccaagacacatatggtttctaaatctatcaagtggtggggatcc
atagttatgacgcagtaacatagattattacattcttgactgtcgctaatatctaaatatttattgttatcgtattggattctgcat
atagatggcttgtatgtcaaagatatagaacacataaccaatttatagtcgcgctttacattctcgaatctaaagttaagagattta
gaaaacattatatcctcggatgatgttatcactgtttctggagtaggatatattaaagtctttacagatttcgtccgattcaaataa
atcactaaataatatcccacattatcatctgttagagtagtatcattaaatctattatattttatgaaagatatatcactgctcacc
tctatatttcgtacatttttaaactgtttgtataatatctctctgatacaatcagatatatctattgtgtcggtagacgataccgtt
acatttgaattaatggtgttccattttacaacttttaacaagttgaccaattcatttctaatagtatcaaactctccatgattaaat
attttaatagtatccattttatatcactacggacacaaagtagctgacataaaccattgtataatttttatgttttatgtttattag
cgtacacattttggaagttccggcttccatgtatttcctggagagcaagtagatgatgaggaaccagatagtttatatccgtacttg
cacttaaagtctacattgtcgttgtatgagtatgatcttttaaacccgctagacaagtatccgtttgatattgtaggatgtggacat
ttaacaatctgacacgtgggtggatcggaccattctcctcctgaacacaggacaccagagttaccaatcaacgaatatccactattg
caactataagttacaacgctcccatcggtataaaaatcctcgtatccgttatgtcttccgttggatatagatggaggggattggcat
ttaacagattcacaaataggtgcctcgggattccataccatagatccagtagatcctaattcacaatacgatttagattcaccgatc
aaatgatatccgctattacaagagtacgttatactagagccaaagtctactccaccaatatcaagttggccattatcgatatctcga
ggcgatgggcatctccgtttaatacattgattaaagagtgtccatccagtacctgtacatttagcatatataggtcccattttttgc
tttctgtatccaggtagacatagatattctatagtgtctcctatgttgtaattagcattagcatcagtctccacactattcttaaat
ttcatattaatgggtcgtgacggaatagtacagcatgatagaacgcatcctattcccaacaatgtcaggaacgtcacgctctccacc
ttcatatttatttatccgtaaaaatgttatcctggacatcgtacaaataataaaaagcccatatatgttcgctattgtagaaattgt
ttttcacagttgctcaaaaacgatggcagtgacttatgagttacgttacactttggagtctcatctttagtaaacatatcataatat
tcgatattacgagttgacatatcgaacaaattccaagtatttgattttggataatattcgtattttgcatctgctataattaagata
taatcaccgcaagaacacacgaacatctttcctacatggttaaagtacatgtataattctatccatttgtcttccttaactatatat
ttgtatagataattacgagtctcgtgagtaattccagtaattacatagatgtcgccgtcgtactctacagcataaactatactatga
tgtctaggcatgggagacttttttatccaacgatttttagtgaaacattccacatcgtttaatactacatatttttcatacgtggta
taaactccacccattacatatatatcatcgtttacgaataccgacgcgcctgaatatctaggagtaattaagtttggaagtcttatc
catttcgaagtgccgtgtttcaaatattctgccacacccgttgaaatagaaaattctaatcctcctattacatataactttccatcg
ttaacacaagtactaacttctgattttaacgacgacatattagtaaccgttttccattttttcgtttcaagatctacccgcgatacg
gaataaacatgtctattgttaatcatgccgccaataatgtatagacaattatgtaaaacatttgcattatagaattgtctatctgta
ttaccgactatcgtccaatattctgttctaggagagtaatgggttattgtggatatataatcagagtttttaatgactactatatta
tgttttataccatttcgtgtcactggctttgtagatttggatatagttaatcccaacaatgatatagcattgcgcatagtattagtc
ataaacttgggatgtaaaatgttgatgatatctacatcgtttggatttttatgtatccactttaataatatcatagctgtaacatcc
tcatgatttacgttaacgttttcgtgggataagatagttgtcagttcatcctttgataattttccaaattctggatcggatgtcacc
gcagtaatattgttgattatttctgacatcgacgcattatatagttttttaattccatatcttttagaaaagttaaacatccttata
caatttgtggaattaatattatgaatcatagtttttacacatagatctactacaggcggaacatcaattattatggcagcaactagt
atcatttctacattgtttatggtgatgtttatcttcttccagcgtatatagtctaatagcgattcaaacgcgtgatagtttatacca
ttcaatataatcgcttcatcctttagatggtgatcctgaattcgtttaaaaaaattatacggagatgccgtaataatttccttattc
acttgtataatttccccattgatagaaaatatcacgctttccattcttgaagtactataagtaattatagtataatgtaaaggttta
tatattcaatattttttataaaaaaatcattttgacattaattcctttttaaatttccgtctatcatctatagaaacgtattctatg
aatttataaaatgcttttacgtgtcctatcgtaggcgatagaaccgctaaaaagcctatcgaatttctacaaaagaatctgttatat
ggtatagggagagtataaaacattaaatgtccgtacttattaaagtattcagtagccaatcctaactctttcgaatacttattaatg
gctcttgttctgtacgaatctatttttttgaacaacggacctagtggtatatcttgttctatgtatctaaaataatgtctgactaga
tccgttagtttaatatcctcagtcatcttgtctagaatggcaaatctaactgcgggtttaggctttagtttagtttttatatctaca
tctatgtctttatctaacaccaaaaatataatagctaatattttattacaatcatccggatattcttctacgatctcactaactaat
gtttctttggttatactagtatagtcactatcggacaaataaagaaaatcagatgatcgatgaataatacatttaaattcatcatct
gtaagatttttgagatgtctcattaaaatattattagggtcagtactcattatcattcggcagctattacttattttatttttctgt
attttattatttttcaccatatagatcaatcattagatcatcaaaatatgtttcaatcatcctaaagagtatggtgaatgactcttc
ccatctaatttctgaacgttcaccaatgtctctagccactttggcactaatagcgatcattcgcttagcgtcttctatattattaac
tggttgattcaatctatctagcaatggaccgtcggacagcgtcattctcatgttcttaatcaatgtacatacatcgccgtcatctac
caattcatccaacaacataagctttttaaaatcatcattataataggtttgatcgttgtcatttctccaaagaatatatctaataag
tagagtcctcatgcttagtaatttaactattttagttaacaactattttttatgttaaatcaattagtacaccgctatgtttaatac
ttattcatattttagtttttaggattgagaatcaatacaaaaaattaatgcatcattaattttagaaatacttagtttccacgtagt
taatgaaacatttgaactcatcgtacaggacgttctcgtacaggacgtaactataaaccggtttatatttgttcaagatagatacaa
atccgataactttttttacgaattctacgggatccactttaaaagtgtcataccgggttctttttattcttttaaacagatcaatgg
tgtgatgttgattaggtcttttacgaatttgatatagaatagcgttcacatatcctccataatggtcaatcgccatttgttcgtatg
tcataaattctttaattatatgacactgtgtattatttagttcatccttgttcatcattaggaatctatccaaaatggcaattatac
tagaactataggtgcgttgtatacacatattgatgtgtctgtttatacaatccatgatatttggatccatgctactaccttcgggta
aaattgtagcatcatataccatttctagtactttaggttcattattatccattgcagaggacgtcatgatcgaatcctaaaaaaata
tattatttttatgttattttgttaaaaataatcatcgaatacttcgtaagatactccttcatgaacataatcagttacaaaacgttt
atatgaagtaaagtatctacgatttttacaaaagtccggatgcataagtacaaagtacgcgataaacggaataataatagatttatc
tagtctatctttttctatagctttcatagttagatacatggtctcagaagtaggattatgtaacatcagcttcgataaaatgactgg
gttatttagtcttacacattcgctcatacatgtatgaccgttaactacaaagtctacactaaaatgattgaacaatagatagtctac
cattgtttcgtattcagatagtacagcgtagtacatagcatcttcacaaattatatcattgtctaatagatatttgacgcatcttat
ggatcccacttcaacagccatcttaaaatcggtagaatcatattgctttcctttatcattaataatttctaaaacatcatctctatc
ataaaagatacaaatattaactgtttgatccgtaataacattgctagtcgatagcaatttgttaataagatgcgctgggctcaatgt
cttaataagaagtgtaagaggactatctccgaatttgttttgtttattaacatccgttgatggaagtaaaagatctataatgtctac
attcttgactgttttagagcatacaatatggagaggtgtatttccatcatgatctggttttgagggactaattcctagtttcatcat
ccatgagattgtagaagcttttggattgtctgacataagatgtctatgaatatgatttttgccaaatttatccactatcctggcttc
gaatccgatggacattatttttttaaacactctttctgaaggatctgtacacgccaacaacggaccacatccttcttcatcaaccga
gttgttaatcttggctccatactgtaccaataaatttattctctctatgacttcatcatctgttcccgagagataatatagaggtgt
tttattatgtttatcacacgcgtttggatctgcgccgtgcgtcagcagcatcgcgactattctattattattaattttagaagctat
atgcaatggataatttccatcatcatccgtctcatttggagagtatcctctatgaagaagttcttcgacaaatcgttcatctagtcc
tttaattccacaatacgcatgtagaatgtgataattatttccagaaggttcgatagcttgtagcatattcctaaatacatctaaatt
tttactattatatttggcataaagagatagataatactcggccgacataatgttgtccattgtagtataaaaattaatatttctatt
tctatttctgtatatttgcaacaatttactctctataacaaatatcataacttagttcttttatgtcaagaaggcactggtttagtt
catctataaatgtcacgccataactaccacgcatgctatactcagaattatgataaagatatttatccttggggtgtaggtaatggg
gattaatctttgttggatcagtctctaagttaacacatgtcacacatgatccatttatagttatatcacacgatgatgatttatgaa
ttgattccggaagatcgctatcgtattttgtggttccacaattcatttccatacatgttattgtcacactaatattatgatgaactt
tatctagccgctgagtggtaaacaacagaacagatagtttattatctttaccaacaccctcagccgctgccacaaatctctgatccg
tatccatgatggtcatgtttatttctagtccgtatccagtcaacactatgttagcatttctgtcgatatagctttcactcatatgac
actcaccaataatagtagaattaatgtcgtaatttacaccaatagtgagttcggcggcaaagtaccaataccggtaatcttgtcgag
gaggacatatagtattcttgtattctaccgaatacccgagagatgcgatacaaaagagtaagactaatttgtaaaccatcttactca
aaatatgtaacaatagtacgatgcaatgagtaagacaataggaaatctatcttatatacacataattattctatcaattttaccaat
tagttagtgtaatgttaacaaaaatgtgggagaatctaattagtttttctttacacaattgacgtacatgagtctgagttccttgtt
tttgctaattatttcatccaatttattattcttgactatatcgagatcttttgtataggagtcagacttgtattcaacatgcttttc
tataatcattttagctatttcggcatcatccaatagtacattttccagattagcagaatagatattaatgtcgtatttgaacagagc
ctgtaacatctcaatgtctttattatctatagccaatttaatgtccggaatgaagagaagggaattattggtgtttgtcgacgtcat
atagtcgagcaagagaatcatcatatccacgtgtccattttttatagtgatgtgaatacaactaaggagaatagccagatcaaaagt
agatggtatctctgaaagaaagtaggaaacaatacttacatcattaagcatgacggcatgataaaatgaagttttccatccagtttt
cccatagaacatcagtctccaatttttcttaacaaacagttttaccgtttgcatgttaccactatcaaccgcataatacaatgcggt
gtttcccttgtcatcaaattgtgaatcatccagtccactgaatagcaaaatctttactattttggtatcttccaatgtggctgcctg
atgtaatggaaattcattctctagaagatttttcaatgctccagcgttcaacaacgtacatactagacgcacgttattatcagctat
tgcataatacaaggcactatgtccatggacatccgccttaaatgcatctttgctagagagaaagcttttcagctgcttagacttcca
agtattaattcgtgacagatccatgtctgaaacgagacgctaattagtgtatattttttcattttttataattttgtcatattgcac
cagaattaataatatctctaatagatctgattagtagatacatggctatcgcaaaacaacatatacacatttaataaaaataatatt
tattaagaaaattcagatttcacgtacccatcaatataaataaaataatgattccttacaccgtacccatattaaggagattccacc
ttacccataaacaatataaatccagtaatatcatgtctgatgatgaacacaaatggtgtattaaattccagtttttcaggagatgat
ctcgccgtagctaccataatagtagatgcctctgctacagttccttgttcgtcgacatctatctttgcattctgaaacattttataa
atatataatgggtccctagtcatatgtttaaacgacgcattatctggattaaacatactaggagccatcatttcggctatcgactta
atatccctcttattttcgatagaaaatttagggagtttaagattgtacactttattccctaattgaaacgaccaatagtctaatttt
gcagccgtaatagaatctgtgaaatgggtcatattatcacctattgccaggtacatactaatattagcatccttatacggaaggcgt
accatatcatattcttcgtcatcgattgtgattgtatttccttgcaatttagtaactacgttcatcatgggaaccgttttcgtaccg
tacttattagtaaaactagcattgcgtgttttagtgatatcaaacggatattgccatatacctttaaaatatatagtattaatgatt
gcccatagagtattattgtcgagcatattagaatctactacattagacataccggatctacgttctactatagaattaattttatta
accgcatctcgtctaaagtttaatctatataggccgaatctatgatattgttgataatacgacggtttaatgcacacagtattatct
acgaaactttgataagttagatcagtgtacgtatatttagatgttttcagcttagctaatcctgatattaattctgtaaatgctgga
cccagatctctttttctcaaatccatagtcttcaataattctattctagtattacctgatgcaggcaatagcgacataaacatagaa
aacgaataaccaaacggtgagaagacaatattatcatcttgaatatttttatacgctactataccggcattggtaaatccttgcaga
cgataggtagacactgaacacgttaacgatagtatcaataacgcaatcatgattttatggtattaataattaaccttatttttatgt
tcggtataaaaattattgatgtctacacatccttttgtaattgacatctatatatccttttgtataatcaactctaatcactttaac
ttttacagttttccctaccagtttatccctatattcaacatatctatccatatgcatcttaacactctctgccaagatagcttcaaa
gtgaggatagtcaaaaagataaatatatagagcataatccttctcgtatactctgccctttattacatcacccgcattgggcaacga
ataacaaaatgcaagcatcttgttaacgggctcgtaaattgggataaaaattatgtttttatatctattttattcaagagaatattc
aggaatttctttttccggttgtatctcatcgcagtatatatcatttgtacattgtttcatattttttaatagtctacaccttttagt
aggactagtatcgtacaattcatagctgtattttgaattccaatcacgcataaaaatatcttccaattgttgacgaagacctaatcc
atcatccggtgtaatattaatagatgctccacatgtatccgtaaagtaatttcctgtccaatttgaggtacctatatacgccgtttt
atcggttaccatatatttggcatggtttaccctagaatacggaatgggaggatcagcatctggtacaataaatagctttacttctat
atttatgtttttagattttagcatagcgatagatcttaaaaagtttctcatgataaacgaagatcgttgccagcaactaatcaatag
cttaactgacacttgtctgtctatagcggctcttcttaattcatcttctatataaggccaaaacaaaatattgcctgccttcgaata
aataatagggataaagttcataacagatacataaacgaatttactcgcatttctgatacatgacaataaagcggttaaatcattggt
tctttccatagtacatagttgttgcggtgcagaagcaataaatacagagtgtggaacgccgcttacgttaatactaagaggatgatc
tgtattataatacgacggataaaagtttttccaattatatggtagattgttaactccaagataccagtatacctcaaaaatttgagt
gagatccgctgccaagttcctattattgaagatcgcaatacccaattctttgacctgagttagtgatctccaatccatgttagcgct
tcctaaataaatatgtgtattatcagatatccaaaattttgtatgaagaactcctcctaggatatttgtaatatctatgtatcgtac
ttcaactccggccatttgtagtctttcaacatcctttaatggtttgttagatttattgacggctactctaactcgtactcctctttt
gggtaattgtacaatcttgtttaatattatcgtgccgaaattcgtacccacttcatccgataaactccaataaaaagatgatatatc
tagtgtttttgtggtattggatagaatttccctccacatgttaaatgtagacaaatatactttatcaaattgcatacctataggaat
agtctctgtaatcactgcgattgtattatccggattcattttatttgttaaaagaataatcctatatcacttcactctattaaaaat
ccaagtttctatttctttcatgactgattttttaacttcatccgtttccttatgaagatgatgtttggcaccttcataaatttttat
ttctctattacaatttgcatgttgcatgaaataatatgcacctaaaacatcgctaatcttattgtttgttccctggagtatgagagt
cggggggtgttaatcttggaaattatttttctaaccttgttggtagccttcaagacctgactagcaaatccagccttaattttttca
tgattgattaatgggtcgtattggtatttataaactttatccatatctctagatactgattctggacatagctttccgactggcgca
tttagtgtgatggttcccataagtttggcagctagcagattcagttttgaaacagcatctgcattaactagaggagacattagaatc
attgctgtaaacaagtttggattatcgtaagaggctagctcccatggaatgacccaataagtagatttaatagttaccacgtgctgt
accaaagtcatcaatcatcattttttcaccattacttcttccatgtccaatatgatcatgtgagaatactaaaattcctaacgatga
tatgttttcagctagttcgtcataacgtccagaatgtttaccagctccatgacttatgaatactaatgccttaggatatgtaatagg
tttccaatatatgtaatcattgtccagattgaacatacagtttgcactcatgattcacgttatataactatcaatattaacagttcg
tttgatgatcatattatttttatgttttattgataattgtaaaaacatacaattaaatcaatatagaggaaggagacggctactgtc
ttttgtgagatagtcatggcgactaaattagattatgaggatgctgttttttactttgtggatgatgataaaatatgtagtcgcgac
tccatcatcgatctaatagatgaatatattacgtggagaaatcatgttatagtgtttaacaaagatattaccagttgtggaagactg
tacaaggaattgatgaagttcgatgatgtcgctatacggtactatggtattgataaaattaatgagattgtcgaagctatgagcgaa
ggagaccactacatcaattttacaaaagtccatgatcaggaaagtttattcgctaccataggaatatgtgctaaaatcactgaacat
tggggatacaaaaagatttcagaatctagattccaatcattgggaaacattacagatctgatgaccgacgataatataaacatcttg
atactttttctagaaaaaaaattgaattgatgatataggggtcttcataacgcataattattacgttagcattctatatccgtgtta
aaaaaaattatcctatcatgtatttgagagttttatatgtagcaaacatgatagctgtgatgccaataagctttagatattcacgcg
tgctagtgttagggatggtattatctggtggtgaaatgtccgttatataatctacaaaataatcatcgcatatagtatgcgatagta
gagtaaacatttttatcgtttctactgggttcatacatcgtctacccaattcggttataaatgaaattgtcgccaatcttacaccca
accccttgttatccattagtatagtattaacttcgttatttatgtcataaactgtaaatgattttgtagatgccatatcatacatga
tattcatgtccctattataatcattactaactttatcacaatatatgttgataatatctatatatgatctagtctttgtgggcaact
gtctatacaagtcgtctaaacgttgtttactcatatagtatcgaacagccatcattacatggtcccgttccgttgatagataatcga
gtatgttagtggacttgtcaaatctatataccatattttctggaagtggatatacatagtcgtgatcaacattattgctagcctcat
cttctatatcctgtactataccattatctatatcatctacataatctacgatattattacacataaacatcgacaacatactattgt
ttattatctaagtcctgttgatccaaacccttgatctcctctatttgtactatctagagattgtacttcttccagttctggataata
tatacgttgatagattagctgagctattctatctccagtatttacattaaacgtacattttccattattaataagaatgactcctat
gtttcccctataatcttcgtctattacaccgcctcctatatcaatgccttttagggacagaccagacctaggagctattctaccata
gcagaacttaggcatggacatactaatatctgtcttaattaactgtcgttctcctggagggatagtataatcgtaagcgctatataa
atcatatccggcggcgtaaggtgattgcctagtaggagatttagctctgttagtttccttaacaaatctaactggtgagttaatatt
catgttgaacataaaactaatattttatttcaaaattatttaccatcccatatattccatgaataagtgtgatgattgtacacttct
atagtatctatatacgattcacgataaaatcctcctatcaatagcagtttattatccactatgatcaattctggattatccctcgga
taaataggatcatctatcagagtccatgtattgctggattcacaataaaattccgcatttctaccaaccaagaataaccttctaccg
aacactaacgcgcatgatttataatgaggataataagtggatggtccaaactgccactgatcatgattgggtagcaaatattctgta
gttgtatcagtttcagaatgtcctcccattacgtatataacattgtttatggatgccactgctggattacatctaggtttcagaaga
ctcggcatattaacccaagcagcatccccgtggaaccaacgctcaacagatgtgggatttggtagacctcctactacgtataattta
ttgttagcgggtatcccgctagcatacagtctggggctattcatcggaggaattggaatccaattgtttgatatataatttaccgct
atagcattgttatgtatttcattgttcatccatccaccgatgagatatactacttctccaacatgagtacttgtacacatatggaat
atatctataatttgatccatgttcataggatactctatgaatggatacttgtatgatttgcgtggttgtttatcacaatgaaatatt
ttggtacagtctagtatccattttacattatttatacctctgggagaaagataatttgacctgattacatttttgataaggagtagc
agatttcctaatttatttcttcgctttatataccacttaatgacaaaatcaactacataatcctcatctggaacatttagttcatcg
ctttctagaataagtttcatagatagataatcaaaattgtctatgatgtcatcttccagttccaaaaagtgtttggcaataaagttt
ttagtatgacataagagattggatagtccgtattctatacccatcatgtaacactcgacacaatattcctttctaaaatctcgtaag
ataaagtttatacaagtgtagatgataaattctacagaggttaatatagaagcacgtaataaattgacgacgttatgactatctata
tatacctttccagtatacgagtaaataactatagaagttaaactgtgaatgtcaaggtctagacaaacccttgtaactggatcttta
tttttcgtgtatttttgacgtaaatgtgtgcgaaagtaaggagataactttttcaatatcgtagaattgactattatattgccacct
atagcatcaataattgttttgaatttcttagtcatagacaatgctaatatattcttacagtacacagtattaacaaatatcggcatt
tatgtttctttaaaagtcaacatctaaagaaaaatgattatcttcttgagacataactcccattttttggtattcacccacacgttt
ttcgaaaaaattagtttttccttccaatgatatattttccatgaaatcaaacggattggtaacattataaatttttttaaatcccaa
ttcagaaatcaatctatccgcgacgaattctatatatgttttcatcatttcacaattcattcctataagtttaactggaagagccgc
agtaagaaattcttgttcaatggataccgcatctgttataatagatctaacggtttcttcactcggtggatgcaataaatgtttaaa
catcaaacatgcgaaatcgcagtgcagaccctcgtctctactaattagttcgttggaaaacgtgagtccgggcattaggccacgctt
tttaagccaaaatatggaagcgaatgatccagaaaagaagattccttctactgcagcaaaggcaataagtctctctccataaccggc
gctgtcatgtatccacttttgagcccaatcggccttcttttttacacaaggcatcgtttctatggcattaaagagatagtttttttc
attactatctttaacataagtatcgatcaaaagactatacatttccgaatgaatgttttcaatggccatctgaaatccgtagaaaca
tctagcctcggtaatctgtacttctgtacaaaatcgttccgccaaattttcattcactattccgtcactggctgcaaaaaacgccaa
tacatgttttataaaatatttttcgtctggtgttagtttattccaatcattgatatctttagatatatctacttcttccactgtcca
aaatgatgcctctgcctttttatacatgttccagatgtcataatattggattgggaaaataacaaatctatttggatttggtgcaag
gatgggttccataactaaattaacaataacaataaattttttttcagttatctatatgcctgtacttggatcttttgtacatcgata
tcgccgcaatcactacaataattacaagtattattgatagcattgttattagtactatcataattaaattatcgacattcatgggtg
ctgaataatcgttattatcatcattatcattttgtaattgtgacatcatactagataaatcgtttgcgagattgttgtgggaagcgg
gcatggaggatgaattatcgttattattatttaacgcctcccattcggattcacaaatgttacgcacattcaacattttatggaaac
tataattttgtgaaaacagataacaagaaaactcgtcatcgttcaaatttttaacgatagtaaaccgattaaacgtcgagctaattt
ctaacgctagcgactctgttggatatgggtttccagatatatatcttttcagttcccctacgtatctataatcatctgtaggaaatg
gaagatatttccatttatctactgttcctaatatcatatgtggtggtgtagtagaaccattaagcgcgaaagatgttatttcgcatc
gtattttaacttcgcaataatttctggttagataacgcactctaccagtcaagtcaatgatattagcctttacagatatattcatag
tagttgtaacgatgactccatcttttagatgcgatactcctttgtatgtaccagaatcttcgtaccgcaaactcgatatatttaaac
aagttaatgagatattaacgcgttttatgaatgatgatatataaccagaagttttatcctcggtggctagcgctataaccttatcat
tataataccaactagtgtgattaatatgtgacacgttagtgtgggtacaaatatgtacattatcgtctacgtcgtattcgatacatc
cgcatacagccaacaaatataaaatgacaaatactctaacgccgttcgtacccatcttgatgcggtttaataaatgttttgatttca
atttattgtaaaaaaagattcggttttatactgttcgatattctcattgcttatattttcatctatcatctccacacagtcaaatcc
gtggttagcatgcacctcatcaaccggtaaaagactatcggactcttctatcattataactctagaatatttaatttggtcattatt
aatcaagtcaattatcttatttttaacaaacgtgagtattttactcattttttataaaaacttttagaaatatacagactctatcgt
gtgtctatatcttctttttatatccaatgtatttatgtctgatttttcttcatttatcatatataatggtccaaattctacacgtgc
ttcggattcatccagatcattaaggttcttataattgtaacatccttctcttccctcttctacatcttccttcttattcttattctt
agcgtcacagaatctaccacagcaggatcccatgacgagcgtcatattaaactaatccattttcaattataatatacgattagtaat
gaccattaaaataaaaaatattcttcataaccggcaagaaagtgaaaagttcacattgaaactatgtcagtagtatacatcatgaaa
tgatgatatatatatactctattttggtggaggattatatgatataattcgtggataatcattcttaagacacatttcttcattcgt
aaatcttttcacgttaaatgagtgtccatattttgcaatttcttcatatgatggcggtgtacgtggacgaagctgctcctgttcttg
ttgtagtcgccgactgtcgtgtttgcgtttagatccctccattatcgcgattgcgtagatggagtactattatataccttgtaatta
aatttttttattaattaaacgtataaaaacgttccgtatctgtatttaagagccagatttcgtctaatagaacaaatagctacagta
aaaataactagaataattgctacacccactagaaaccacggatcgtaatacggcaatcggttttcgataataggtggaacgtatatt
ttatttaaggacttaacaattgtctgtaaaccacaatttgcttccgcggatcctgtattaactatctgtaaaagcatatgttgaccg
ggcggagccgaacattctccgatatctaatttctgtatatctataatattattaacctccgcatacgcattacagttcttttctagc
ttggataccgcactaggtacatcgtctagatctattcctatttcttcagcgatagctcttctatccttttccggaagcaatgaaatc
acttcaataaatgattcaaccatgagtgtgaaactaagtcgagaattactcatgcatttgttagttattcggagcgcgcaattttta
aactgtcctataacctctcctatatgaatagcacaagtgacattagtagggatagaatgttgagctaatttttgtaaataactatct
ataaaaagattatacaaagttttaaactctttagtttccgccatttatccagtctgagaaaatgtctctcataataaatttttccaa
gaaactaattgggtgaagaatggaaacctttaatctatatttatcacagtctgttttggtacacatgatgaattcttctaatgctgt
actaaattcgatatctttttcgatttctggatatgtttttaataaagtatgaacaaagaaatggaaatcgtaataccagttatgttc
aactttgaaattgttttttattttcttgttaatgattccagccacttgggaaaagtcaaagtcgtttaatgccgatttaatacgttc
attaaaaacaaactttttatcctttagatgaattattattggttcattggaatcaaaaagtaagatattatcgggtttaagatctgc
gtgtaaaaagttgtcgcaacagggtagttcgtagattttaatgtataacagagccatctgtaaaaagataaactttatgtattgtac
caaagatttaaatcctaatttgatagctaactcggtatctactttatctgccgaatacagtgctaggggaaaaattataatatttcc
tctttcgtattcgtagttagttctcttttcatgttcgaaaaagtgaaacatgcggttaaaatagtttataacattaatattactgtt
aataactgccggataaaagtgggatagtaatttcacgaatttgatactgtcctttctctcgttaaacgcctttaaaaaaactttaga
agaatatctcaatgagagttcctgaccatccatagtttgtatcaataatagcaacatatgaagaacccgtttatacagagtatgtaa
aaatgttaatttatagtttaatcccatggcccacgcacacacgattaattttttttcatctccctttagattgttgtatagaaattt
gggtactgtgaactccgccgtagtttccatgggactatataattttgtggcctcgaatacaaattttactacatagttatctatctt
aaagactataccatatcctcctgtagatatgtgataaaaatcgtcgtttataggataaaatcgtttatccttttgttggaaaaagga
tgaattaatgtaatcattctcttctatctttagtagtgtttccttattaaaattcttaaaataatttaacaatctaactgacggagc
ccaattttggtgtaaatctaattgggacattatgttgttaaaatacaaacagtctcctaatataacagtatctgataatctatgggg
agacatccattgatattcaggggatgaatcattggcaacacccatttattgtacaaaaagccccaatttacaaacgaaagtccaggt
ttgatagagacaaacaattaactattttgtctctgtttttaatttctttggtaatgaaattattcacaatatcagtatcttctttat
ctaccagagattttactaacttgataaccttggctgtctcattcaatagggtagtaatatttgtatgtgtgatattgatatcttttt
gaattgtttcttttagaagtgattctttgatggtgccagcatacgaattacaataatgcagaaactcagttaacatgcaggaattat
agtaagccaattccaattgttgcctgtattgtattagagtattaatatgcgcaatggtgtccttgcgtttctctgatagaatgcgag
cagcgattttggcgttatcatttgacgatatttctggaatgacgaatcctgtttctactaactttttggtaggacaaagtgaaacaa
tcaagaagatagcttctcctcctatttgtggaagaaattgaactcctctagatgatctactgacgatagtatctccttgacagatat
tggaccgaattacagaagtacctggaatgtaaagccctgaaaccccctcattttttaagcagattgttgccgtaaatcctgcactgt
gaccaagatagagagctcctttggtgaatccatctctatgtttcagtttaaccaagaaacagtcagctggtctaaaatttccatctc
tatctaatacagcatctaacttgatgtcaggaactatgaccggtttaatgttatatgtaacattgagtaaatccttaagttcataat
catcactgtcatcagttatgtacgatccaaacaatgtttctactggcatagtggatacgaagatgctatccatcagaatgtttccct
gattagtattttctatatagctattcttctttaaacgattttccaaatcagtaactatgttcatttttttaggagtaggacgcctag
ccagtatggaagaggattttctagatcctctcttcaacatctttgatctcgatggaatgcaaaaccccatagtgaaacaaccaacga
taaaaataatattgtttttcactttttataattttaccatctgactcatggattcattaatatctttataagagctactaacgtata
attctttataactgaactgagatatatacaccggatctatggtttccataattgagtaaatgaatgctcggcaataactaatggcaa
atgtatagaacaacgaaattatactagagttgttaaagttaatattttctatgagctgttccaataaattatttgttgtaactgcgt
tcaagtcataaatcatcttgatactatccagtaaaccgtgtttaagttctggaatattatcatcccattgtaaagcccctaattcga
ctatcgaatatcctgctctgatagcagtttcaatatcgacggacgtcaatactgtaataaaggtggtagtattgtcatcatcgtgat
aaactacgggaatatggtcgttagtaggtacggtgactttacacaacgcgatatataactttccttttgtaccatttttaacgtagt
tgggacgtcctgcagggtattgttttgaagaaatgatatcgagaacagatttgatacgatatttgttggattcctgattattcacta
taatataatctagacagatagatgattcgataaatagagaaggtatatcgttggtaggataatacatccccattccagtattctcgg
atactctattgatgacactagttaagaacatgtcttctattctagaaaacgaaaacatcctacatggactcattaaaacttctaacg
ctcctgattgtgtctcgaatgcctcgtacaaggatttcaaggatgccatagattctttgaccaacgatttagaattgcgtttagcat
ctgatttttttattaaatcgaatggtcggctctctggtttgctaccccaatgataacaatagtcttgtaaagataaaccgcaagaaa
atttatacgcatccatccaaataaccctagcaccatcggatgatattaatgtattattatagattttccatccacaattattgggcc
agtatactgttagcaacggtatatcgaatagattactcatgtaacctactagaatgatagttcgtgtactagtcataatatctttaa
tccaatctaagaaatttaaaattagattttttacactgttaaagttaacaaaggtattacccggatacgtggatatcatatatggca
ttggtccattatcagtaatagctccataaactgatacggcgatggtttttatatgtgtttgatctaacgaggaagaaattcgcgccc
acaattcatctctagatatgtatttaatatcaaacggtaacacatcaatttcgggacgcgtatatgtttctaaatttttaatccaaa
tataatgatgacctatatgccctattatcatactgtcaactatagtacacctagagaacttacgatacatctgtttcctataatcgt
taaattttacaaatctataacatgctaaaccttttgacgacagccattcattaatttctgatatggaatctgtattctcgataccgt
attgttctaaagccagtgctatatctccctgttcgtgggaacgctttcgtataatatcgatcaacggataatctgaagtttttggag
aataatatgactcatgatctatttcgtccataaacaatctagacataggaattggaggcgatgatcttaattttgtgcaatgagtcg
tcaatcctataacttctaatcttgtaatattcatcatcgacataatactatctatgttatcatcgtatattagtataccacggcctt
cttcatttcgtgccaaaataatatacagtcttaaataattacgcaatatctcaatagtttcataattgttagctgttttcatcaaga
tttgtaccctgtttaacatgatggcgttctatacgtctctattttcttttttttaaatttttaacgatttactgtggctagataccc
aatctctctcaaatatttttttagcctcgcttacaagctgtttatctatactattaaaactgacgaatccgtgattttggtaatggg
ttccgtcgaaatttgccgaagtgatatgaacatattcgtcgtcgactatcaacaattttgtattattctgaatagtgaaaaccttca
cagatagatcattttgaacacacaacgcgtctagacttctggcggttgccatagaatatacgtcgttcttatcccaattaccaacta
gaagtctgatcttaactcctctattaatggctgcttctataatggagttgtaaatgtcgggccaatagtagctattaccgtcgacac
gtgtagtgggaactatggccaaatgttcaatatctatactagtcttagccgacttgagtttatcaataactacatcggtatctagat
ctctagaatatcccaataggtgttccggagaatcagtaaagaacactccacctataggattcttaatatgatacgcagtgctaactg
gcaaacaacaagccgcagagcataaattcaaccatgaattttttgcgctattaaaggctttaaaagtatcaaatcttctacgaagat
ctgtggccagcgggggataatcagaatatacacctaacgttttaatcgtatgtatagatcctccagtaaatgacgcgtttcctacat
aacatctttcatcatctgacacccaaaaacaaccgagtagtagtcccacattattttttttatctatattaacggttataaaattta
tatccgggcagtgactttgtagctctcccagatttcttttccctcgttcatctagcaaaactattattttaatccctttttcagatg
cctcttttagtttatcaaaaataagcgctcccctagtcgtactcagaggattacaacaaaaagatgctatgtatatatatttcttag
ctagagtgataatttcgttaaaacattcaaatgttgttaaatgatcggatctaaaatccatattttctggtagtgtttctaccagcc
tacattttgctcccgcaggtaccgatgcaaatggccacatttagttaacataaaaacttatacatcctgttctatcaacgattctag
aatatcatcggctatatcgctaaaattttcatcaaagtcgacatcacaacctaactcagtcaatatattaagaagttccatgatgtc
atcttcgtctatttctatatccgtatccattgtagattgttgaccgattatcgagtttaaatcattactaatactcaatccttcaga
atacaatctgtgtttcattgtaaatttataggcggtgtatttaagttggtagattttcaattatgtatcaatatagcaacagtagtt
cttgctcctccttgattctagcatcctcttcattattttcttctacgtacataagcatgtccaatacgttagacaacacaccgacga
tggcggccgccacagacacgaatatgactagaccgatgaccatttaaaaacccctctctagctttcacttaaactgtatcgattatt
cttttagaacatgtataatataaaaacattattctatttcgaatttaggcttccaaaaatttttcatccgtaaaccgataataatat
atatagacttgttaatagtcggaataaatagattaatgcttaaactatcatcatctccacgattagagatacaatatttacattttt
tttgctgtttcgaaactttatcaatacacgttaatacaaacccaggaaggagatattgaaactgaggctgttgaaaatgaaacggtg
aatacaataattcagataatgtaaaatcatgattccgtattctgatgatattagaactgctaatggatgtcgatggtatgtatctag
gagtatctattttaacaaagcatcgatttgctaatatacaattatccttttgattaattgttattttattcatattcttaaaaggtt
tcatatttatcaattcttctacattaaaaatttccatttttaatttatgtagcccccgcaatactcctcattacgtttcattttttg
tctataatatccattttgttcatctcggtacatagattatccaattgagaagcgcatttagtagttttgtacattttaagtttattg
acgaatcgtcgaaaactagttatagttaacattttattatttgataccctgatattaatacccctgccgttactattatttataact
gatgtaatccacgtaacattagaattaattatcgatagtaatgcatcgacgcttccaaaattgtctattataaactcaccgataatt
tttttattgcatgttttcatattcattaggattatcaaatctttaatcttactacgattgtatgcgttgatattgcaagacgtcatt
ctaaaagacggaggatctccatcaaatgccagacaatcacgtacaaagtacatggaaataggttttgttctattgcgcatcatagat
ttatatagaacacccgtagaaatactaatttgttttactctataaaatactaatgcatctatttcatcgttttgtataacgtctttc
caagtgtcaaattccaaatttttttcattgatagtaccaaattcttctatctctttaactacttgcatagataggtaattacagtga
tgcctacatgccgttttttgaaactgaatagatgcgtctagaagcgatgctacgctagtcacaatcaccactttcatatttagaata
tatgtatgtaaaaatatagtagaatttcattttgtttttttctatgctataaatgaattctcattttgcatctgctcatactccgtt
ttatatcaataccaaagaaggaagatatctggttctaaaagccgttaaagtatgcgatgttagaactgtagaatgcgaaggaagtaa
agcttcctgcgtactcaaagtagataaaccctcatcgcccgcgtgtgagagaagaccttcgtccccgtccagatgcgagagaatgaa
taaccctggaaaacaagttccgtttatgaggacggacatgctacaaaatatgttcgcggctaatcgcgacaacgtggcgtcgagact
tttgaactaaaatacaattatatccttttcgatattaataaatccgtgtcgtccaggttttttatctctttcagtatgtgaatagat
aggtattttatctctattcatcatcgaatttaagagatccgataaacattgtttgtattctccagatgtcagcatctgatacaacaa
tatatgtgcacataaacctctggcacttatttcatgtaccttccccttatcactaaggagaatagtatttgagaaatatgtatacat
gatattatcatgaattagatatacagaatttgtaacactctcgaaatcacacgatgtgtcggcgttaagatctaatatatcactcga
taacacattttcatctagatacactagacattttttaaagctaaaatagtctttagtagtgacagtaactatgcgattattttcatc
gatgatacatttcatcggcatattattacgcttaccatcaaagactataccatgtgtatatctaacgtattctagcatggttgccat
acgcgcattaaacttttcaggatctttggatagatcttccaatctatctatttgagaaaacatttttatcatgttcaatagttgaaa
cgtcggatccactatatagatattatctataaagattttaggaactacgttcatggtatcctggcgaatattaaaactatcaatgat
atgattatcgttttcatcttttatcaccatatagtttctaagatatgggattttacttaatataatattatttcccgtgataaattt
tattagaaaggccaaatctataagaaaagttctagaattagtctgaagaatatctatatcgccgtaccgtatatttggattaattag
atatagagaatatgatccgtaacatatacaacttttattatggcgtctaagatattcttccatcaacttattaacatttttgactag
ggaagatacattatgacgtcccattacttttgccttgtctattactgcgacgttcatagaatttagcatatctcttgccaattcttc
cattgatgttacattataagaaattttagatgaaattacatttggagctttaatagtaagaactcctaatatgtccgtgtatgtggt
cactaatacagattgtagttctataatcgtaaataatttacctatattatatgtttgagtctgtttagaaaagtagctaagtatacg
atcttttatttctgatgcagatgtattaacatcggaaaaaaatctttttttattcttttttactaaagatacaaatatgtctttgtt
aaaaacagttattttttgaatatttctagcttgtaattttaacatatgatattcgttcacactaggtactctgcctaaataggtttc
tataatctttaatgtaatattaggaagagtattctgatcaggattcctattcattttgaggatttaaaactctgattattgtctaat
atggtctctacgcaaactttttcacagagcgatagagtttttgataactcgtttttcttaagaaatataaaactactgtctccagag
ctcgctctatcttttattttatttaattcgatacaaactcctgatactggttcagaaagtaattcattaattttcagtcctttatag
aagatatttaatatagataatacaaaattttcagttcttgatatcgatctgattgatcctagaactagatatattaataacgtgctc
attaggcagtttatggcagcttgataattagatatagtatattccagttcatatttattagataccgcattgcccagattttgatat
tctatgaattcctctgaaaataaatccaaaataactaaacattctattttttgtggattagtgtactctcttccctctatcatgttc
actactggtgtccacgatgataaatatctagagggaatataatatagtccataggatgccaatctagcaatgtcgaataactgtaat
ttgattcttcgttcttcattatgaattgattcttgaggtataaacctaacacaaattatattattagacttttcgtatgtaatgtct
ttcatgttataagtttttaatcctggaatagaatctattttaatgaggcttttaaacgcagagttctccaacgagtcaaagcataat
actctgttgtttttcttatatacgatgttacgattttcttctttgaatggaataggtttttgaattagtttataattacaacataat
agataaggaagtgtgcaaatagtacgcggaaaaaacataatagctcccctgttttcatccatggttttaagtaaatgatcactggct
tctttagtcaatggatattcgaacattaaccgtttcatcatcattggacagaatccatatttcttaatgtaaagagtgatcaaatca
ttgtgtttattgtaccatcttgttgtaaatgtgtattcggttatcggatctgctcctttttctattaaagtatcgatgtcgatctcg
tctaagaattcaactatatcgacatatttcatttgtatacacataaccattactaacgtagaatgtataggaagagatgtaacggga
acagggtttgttgattcgcaaactattctaatacataattcttctgttaatacgtcttgcacgtaatctattatagatgccaagata
tctatataattattttgtaagatgatgttaactatgtgatctatataagtagtgtaataattcatgtatttcgatatatgttccaac
tctgtctttgtgatgtctagtttcgtaatatctatagcatcctcaaaaaatatattcgcatatattcccaagtcttcagttctatct
tctaaaaaatcttcaacgtatggaatataataatctattttacctcttctgatatcattaatgatatagtttttgacactatcttct
gtcaattgattcttattcactatatctaagaaacggatagcgtccctaggacgaactactgccattaatatctctattatagcttct
ggacataattcatctattataccagaattaatgggaactattccgtatctatctaacatagttttaagaaagtcagaatctaagacc
tgatgttcatatattggttcatacatgaaatgatctctattgatgatagtgactatttcattctctgaaaattggtaactcattcta
tatatgctttccttgttgatgaaggatagaatatactcaatagaatttgtaccaacaaactgttctcttatgaatcgtatatcatca
tctgaaataatcatgtaaggcatacatttaacaattagagacttgtctcctgttatcaatatactattcttgtgataatttatgtgt
gaggcaaatttgtccacgttctttaattttgttatagtagatatcaaatccaatggagctacagttcttggcttaaacagatatagt
ttttctggaacgaattctacaacattattataaaggactttgggtagataagtgggatgaaatcctattttaattaatgcgatagcc
ttgtcctcgtgcagatatccaaacgcttttgtgatagtatggcattcattgtctagaaacgctctacgaatatctgtgacagatatc
atctttagagaatatactagtcgcgttaatagtactacaatttgtattttttaatctatctcaataaaaaaattaatatgtatgatt
caatgtataactaaactactaactgttattgataactagaatcagaatctaatgatgacgtaaccaagaagtttatctactgccaat
ttagctgcattatttttagcatctcgtttagattttccatctgccttatcgaatactcttccgtcgatgtctacacaggcataaaat
gtaggagagttactaggccccactgattcaatacgaaaagaccaatctctcctagtaatttggcagtactcattaataacggtgaca
gggttagcacctttccaatcaataatttttttagccggaataacatcatcaaaagacttatgatcctctctcattgatttttcgcgg
gatacatcatctattatgacgtcagccatagcatcagcatccggcttatccgcctccgttgtcataaaccaacgaggaggaatatcg
tcggagctgtacaccatagcactacgttgaagatcgtacagagctttattaacttctcgcttctccatattaagttgtctagttagt
tgtgcagcagtagctccttcgattccaatggttttaatagcctcacacacaatctctgcgttagaacgctcgtcgatatagatttta
gacatttttagagagaactaacgcaatcagtaataaaactaatttattttatcattttttttattcatcatcctctggtggttcgtc
gtttctatcgaatgtagctctgattaacccgtcatctataggtgatgctggttctggagattctggaggagatggattattatctgg
aagaatctctgttatttccttgttttcatgtatcgattgcgttgtaacattaagattgcgaaatgctctaaatttgggaggcttaaa
gtgttgtttgcaatctctacacgcgtgtctaactagtggaggttcgtcagcggctctagtttgaatcatcatcggcgtagtattcct
acttttacagttaggacacggtgtattgtatttctcgtcgagaacgttaaaataatcgttgtaactcacatcctttattttatctat
attgtattctactcctttcttaatgcattttataccgaataagagatagcgaaggaattctttttcggtgccgctagtacccttaat
catatcacatagtgttttatattccaaatttgtggcaatagacggtttatttctatacgatagtttgtttctggaatcctttgagta
ttctataccaatattattctttgattcgaatttagtttcttcgatattagattttgtattacctatattcttgatgtagtactttga
tgatttttccatggcccattctattaagtcttccaagttggcatcatccacatattgtgatagtaattctcggatatcagtagcggt
taccgccattgatgtttgttcattggatgagtaactactaatgtatacattttccatttataacacttatgtattaactttgttcat
ttatattttttcattattatgttgatattaacaaaagtgaatatatatatgttaataattgtattgtggttatacggctacaatttt
ataattagtgaaagtcagtgtccgatgatcaatgacgatagctttactctgaaaagaaagtatcaaatcgatagtgcggagtcaaca
ataaaaatggataagaagaggacaaagtttcagaatagagccaaaatggtaaaagaaataaatcagacaataagagcagcacaaact
cattacgagacattgaaactaggatacataaaatttaagagaatgattaggactactactctagaagatatagcaccatctattcca
aataatcagaaaacttataaactattctcggacatttcagccatcggcaaagcatcacggaatccaagtaagatggtatatgctctg
ctgctttacatgtttcccaatttgtttggagatgatcatagattcattcgttatagaatgcatccaatgagtaaaatcaaacacaag
atcttctctcctttcaaacttaatcttattagaatattagtggaagaaagattctataataatgaatgcagatctaataaatggaga
ataattggaacacaagttgataaaatgttgatagctgaatctgataaatatacaatagatgcaaggtataacctaaaacccatgtat
agaatcaagggaaaatctgaagaagataccctctttatcaaacagatggtagaacaatgtgtgacatcccaggaattggtggaaaaa
gtgttgaagatactgtttagagatttgttcaagagtggagaatacaaagcgtacagatacgatgatgatgtagaaaatggatttatt
ggattggatacactaaaattaaacattgttcatgatatagttgaaccatgtatgcctgttcgtaggccagtggctaagatactgtgt
aaagaaatggtaaataaatactttgagaatccgctacatattattggtaaaaatcttcaagagtgcattgactttgttagtgaatag
gcatttcatctttctccaatactaattcaaattgttaaattaataatggatagtataaatagttattagtgataaaatagtaaaaat
aattattagaataagagtgtagtatcatagataactctcttctataaaaatggattttattcgtagaaagtatcttatatacacagt
agaaaataatatagattttttaaaggatgatacattaagtaaagtaaacaattttaccctcaatcatgtactagctctcaagtatct
agttagcaattttcctcaacatgttattactaaggatgtattagctaataccaatttttttgttttcatacatatggtacgatgttg
taaagtgtacgaagcggttttacgacacgcatttgatgcacccacgttgtacgttaaagcattgactaagaattatttatcgtttag
taacacaatacaatcgtacaaggaaaccgtgcataaactaacacaagatgaaaaatttttagaggttgccaaatacatggacgaatt
aggagaacttataggcgtaaattatgacttagttcttaatccattatttcacggaggggaacccatcaaagatatggaaatcatttt
tttaaaactgtttaagaaaacagacttcaaagttgttaaaaaattaagtgttataagattacttatttgggcttacctaagcaagaa
agatacaggcatagagtttgcggataatgatagacaagatatatatactctatttcaacaaactggtagaatcgtccatagcaatct
aacagaaacgtttagagattatatctttcccggagataagactagctattgggtgtggttaaacgaaagtatagctaatgatgcgga
tattgttcttaatagacacgccattaccatgtatgataaaattcttagttatatatactctgagataaaacaaggacgcgttaataa
aaacatgcttaagttagtttatatctttgagcctgaaaaagatatcagagaacttctgctagaaatcatatatgatattcctggaga
tatcctatctattattgatgcaaaaaacgacgattggaaaaaatattttattagtttttataaagctaattttattaacggtaatac
atttattagtgatagaacgtttaacgaggacttattcagagttgttgttcaaatagatcccgaatatttcgataatgaacgaattat
gtctttattctctacgagtgctgcggacattaaacgatttgatgagttagatattaataacagttatatatctaatataatttatga
ggtgaacgatatcacattagatacaatggatgatatgaagaagtgtcaaatctttaacgaggatacgtcgtattatgttaaggaata
caatacatacctgtttttgcacgagtcggatcccatggtcatagagaacggaatactaaagaaactgtcatctataaaatccaagag
tagacggctgaacttgtttagcaaaaacattttaaaatattatttagacggacaattggctcgtctaggtcttgtgttagatgatta
taaaggagacttgttagttaaaatgataaaccatcttaagtctgtggaggatgtatccgcattcgttcgattttctacagataaaaa
ccctagtattcttccatcgctaatcaaaactattttagctagttataatatttccatcatcgtcttatttcaaaggtttttaagaga
taatctatatcatgtagaagaattcttggataaaagcatccatctaaccaagacggataagaaatatatacttcaattgataagaca
cggtagatcatagaacagaccaaatatattattaataatttgtatatacatagatataattatcacatattaaaaattcacacattt
ttgataaatgggaactgctgcaacaattcagactcccaccaaattaatgaataaagaaaatgcagaaatgattttggaaaaaattgt
tgatcatatagttatgtatattagtgacgaatcaagtgattcagaaaataatcctgaatatattgattttcgtaacagatacgaaga
ctatagatctctcattataaaaagtgatcacgagtttgtaaagctatgtaaaaatcatgcagagaaaagttctccagaaacgcaaca
aatgattatcaaacacatatacgaacaatatcttattccagtatctgaagtactattaaaacctataatgtccatgggtgacataat
tacatataacggatgtaaagacaatgaatggatgctagaacaactctctaccctaaactttaacaatctccgcacatggaactcatg
tagcataggcaatgtaacgcgtctgttttatacattttttagttatctgatgaaagataaactaaatatataagtataatcccattc
taatactttaacctgatgtattagcatcttattagaatattaacctaactaaaagacataacataaaaactcattacatagttgata
aaaagcggtaggatataaatattatggctgccaccgttccgcgttttgacgacgtgtacaaaaatgcacaaagaagaattctagatc
aagaaacattttttagtagaggtctaagtagaccgttaatgaaaaacacatatctatttgataattacgcgtatggatggataccag
aaactgcaatttggagtagtagatacgcaaacttagatgcaagtgactattatcccatttcgttgggattacttaaaaagttcgagt
ttctcatgtctctatataaaggtcctattccagtatacgaagaaaaagtaaatactgaattcattgctaatggatcgttctctggta
gatacgtatcatatcttcgaaagttttctgctcttccaacaaacgagtttattagttttttgttactgacttccattccaatctata
atatcttgttctggtttaaaaatactcagtttgatattactaaacacacattattcagatacgtctatacagataatgccaaacacc
tggcgttggctaggtatatgcatcaaacaggagactataagcctttgtttagtcgtctcaaagagaattatatatttaccggtcccg
ttccaataggtatcaaagatataaatcaccctaatcttagtagagcaagaagtccatccgattatgagacattagctaatattagta
ctatattgtactttaccaagtatgatccggtattaatgtttttattgttttacgtacctgggtattcaattactacaaaaattactc
cagccgtagaatatctaatggataaactgaatctaacaaagagcgacgtacaactgttgtaaattattttatgcttcgtaaaatgta
ggttttgaaccaaacattctttcaaagaatgagatgcataaaactttattatccaatagattgactatttcggacgtcaatcgttta
aagtaaacttcgtaaaatattctttgatcactgccgagtttaaaacttctatcgataattgtttcatatgttttaatatttacaagt
tttttggtccatggtacattagccggacaaatatatgcaaaataatatcgttctccaagttctatagtttctggattatttttatta
tattcagtaaccaaatacatattagggttatctgcggatttataatttgagtgatgcattcgactcaacataaataattctagagga
gacgatctactatcaaattcggatcgtaaatctgtttctaaagaacggagaatatctatacatacctgattagaattcatccgtcct
tcagacaacatctcagacagtctggtcttgtatgtcttaatcatattcttatgaaacttggaaacatctcttctagtttcactagta
cctttattaattctctcaggtacagattttgaattcgacgatgccgagtatttcatcgttgtatatttcttcttcgattgcataatc
agattcttatataccgcctcaaactctattttaaaattattaaacaatactctattattaatcagtcgttctaactcctttgctatt
tctatggacttatctacatcttgactgtctatctctgtaaacacggagtcggtatctccatacacgctacgaaaacgaaatctgtaa
tctataggcaacgatgttttcacaatcggattaatatctctatcgtccatataaaatggattacttaatggattggcaaaccgtaac
ataccgttagataactctgctccatttagtaccgattctagatacaagatcattctacgtcctatggatgtgcaactcttagccgaa
gcgtatgagtatagagcactatttctaaatcccatcagaccatatactgagttggctactatcttgtacgtatattgcatggaatca
tagatggccttttcagttgaactggtagcctgttttaacatctttttatatctggctctctctgccaaaaatgttcttaatagtcta
ggaatggttccttctatcgatctatcgaaaattgctatttcagagatgaggttcggtagtctaggttcacaatgaaccgtaatatat
ctaggaggtggatatttctgaagcaagagctgattatttatttcttcttccaatctattggtactaacaacgacaccgactaatgtt
tccggagatagatttccaaagatacacacattaggatacagactgttataatcaaagattaatacattattactaaacattttttgt
tttggagcaaataccttaccgccttcataaggaaacttttgttttgtttctgatctaactaagatagttttagtttccaacaatagc
tttaacagtggacccttgatgactgtactcgctctatattcgaataccatggattgaggaagcacatatgttgacgcacccgcgtct
gtttttgtttctactccataatactcccacaaatactgacacaaacaagcatcatgaatacagtatctagccatatctaaagctatg
tttagattataatccttatacatctgagctaaatcaacgtcatcctttccgaaagataatttatatgtatcattaggtaaagtagga
cataatagtacgactttaaatccattttcccaaatatctttacgaattactttacatataatatcctcatcaacagtcacataatta
cctgtggttaaaacctttgcaaatgcagcggctttgcctttcgcgtccgtagtatcgtcaccgatgaacgtcatttctctaactcct
ctatttaatactttacccatgcaactgaacgcgttcttggatatagaatccaatttgtacgaatccaatttttcagatttttgaatg
aatgaatatagatcgaaaaatatagttccattattgttattaacgtgaaacgtagtattggccatgccgcctactcccttatgacta
gactgatttctctcataaatacagagatgtacagcttcctttttgtccggagatctaaagataatcttctctcctgttaataactct
agacgattagtaatatatctcagatcaaagttatgtccgttaaaggtaacgacatagtcgaacgttagttccaacaattgtttagct
attcgtaacaaaactatttcagaacataaaactagttctcgttcgtaatccatttccattagtgactgtatcctcaaacatcctcta
tcgacggcttcttgtatttcctgttccgttaacatctcttcattaatgagcgtaaacaataatcgtttaccacttaaatcgatataa
cagtaacttgtatgcgagattgggttaataaatacagaaggaaacttcttatcgaagtgacactctatatctagaaataagtacgat
cttgggatatcgaatctaggtatttttttagcgaaacagttacgtggatcgtcacaatgataacatccattgttaatctttgtcaaa
tattgctcgtccaacgagtaacatccgtctggagatatcccgttagaaatataaaaccaactaatattgagaaattcatccatggtg
gcattttgtatgctgcgtttctttggctcttctatcaaccacatatctgcgacggagcattttctatctttaatatctagattataa
cttattgtctcgtcaatgtctatagttctcatctttcccaacggcctcgcattaaatggaggaggagacaatgactgatatatttcg
tccgtcactacgtaataaaagtaatgaggaaatcgtataaatacggtctcaccatttcgacatctggatttcagatataaaaatctg
ttttcaccgtgactttcaaaccaattaatgcaccgaacatccatttatagaatttagaaatatattttcatttaaatgaatcccaaa
cattggggaagagccgtatggaccattatttttatagtactttcgcaagcgggtttagacggcaacatagaagcgtgtaaacgaaaa
ctatatactatagttagcactcttccatgtcctgcatgtagacggcacgcgactatcgctatagaggacaataatgtcatgtctagc
gatgatctgaattatatttattattttttcatcagattatttaacaatttggcatctgatcccaaatacgcgatcgatgtgacaaag
gttaaccctttataaacttaacccattataaaacttatgattagtcacgactgaaataaccgcgtgattattttttggtataattct
acacggcatggtttctgtgactatgaattcaacccccgttacattagtgaaatctttaacaaacagcaagggttcgtcaaagacata
aaactcattgtttacaatcgaaatagaccccctatcacacttaaaataaaaaatatccttatcctttaccaccaaataaaattctga
ttggtcaatgtgaatgtattcacttaacagttccacaaatttatttattaactccgaggcacatacatcgtcggtattttttatggc
aaactttactcttccagcatccgtttctaaaaaaatattaacgagttccatttatatcatccaatattattgaaatgacgttgatgg
acaaatgatacaaataagaaggtacggtacctttgtccaccatctcctccaattcatgctctattttgtcattaactttaatgtatg
aaaacagtacgccacatgcttccatgacagtgtgtaacactttggatacaaaatgtttgacattagtataattgttcaagactgtca
atctataatagatagtagctataatatattctatgatggtattgaagaagatgacaaccttggcatattgatcatttaacacagaca
tggtatcaacagatagcttgaatgaaagagaatcagtaattggaataagcgtcttctcgatggagtgtccgtataccaacatgtctg
atattttgatgtattccattaaattatttagttttttctttttattctcgttaaacagcatttctgtcaacggaccccaacatcgtt
gaccgattaagttttgattgatttttccgtgtaaggcgtatctagtcagatcgtatagcctatccaataatccatcgtctgtgtgta
gatcacatcgtacactttttaattctctatagaagagcgacagacatctggagcaattacagacagcaatttctttattctctacag
atgtaagatacttgaagacattcctatgatgatgcagaattttggataacacggtattgatggtatctgttaccataattcctttga
tggctgatagtgtcagagcacaagatttccaatctttgacaatttttagcaccattatctttgttttgatatctatatcagacagca
tggtgcgtctgacaacacaaggattaagacggaaagatgaaatgattctctcaacatcttcaatggataccttgctattttttctgg
cattatctatatgtgcgagaatatcctctagagaatcagtatcctttttgatgatagtggatctcaatgacatgggacgtctaaacc
ttcttattctatcaccagattgcatggtgatttgtcttctttcttttatcataatgtaatctctaaattcatcggcaaattgtctat
atctaaaatcataatatgagatgtttacctctacaaatatctgttcgtccaatgttagagtatttacatcagttttgtattccaaat
taaacatggcaacggatttaattttatattcctctattaagtcctcgtcgataataacagaatgtagataatcatttaatccatcgt
acatggttggaagatgctcgttgacaaaatctttaattgtcttgatgaaggtgggactatatctaacatcttgattaataaaattta
taacattgtccataggatactttgtaactagttttatacacatctcttcatcggtaagtttagacagaatatcgtgaacaggtggta
tattatattcatcagatatacgaagaacaatgtccaaatctatattgtttaatatattatatagatgtagtgtagctcctacaggaa
tatctttaactaagtcaatgatttcatcaaccgttagatctattttaaagttaatcatataggcattgatttttaaaaggtatgtag
ccttgactacattctcattaattaaccattccaagtcactgtgtgtaagaagattatattctatcataagcttgactacatttggtc
ccgataccattaaagaattcttatgatataaggaaacagattttaggtactcatctactctacaagaattttggagagccttaacga
tatcagtgacgtttattatttcaggaggaaaaaacctaacattgagaatatcggaattaatagcttccagatacagtgattttggca
atagtccgtgtaatccataatccagtaacacgagctggtgcttgctagacaccttttcaatgtttaatttttttgaaataagctttg
ataaagccttcctcgcaaattccggatacatgaacatgtcggcgacatgattaagtattgttttttcattatttttatattttctca
acaagttctcaataccccaatagatgatagaatatcacccaatgcgtccatgttgtctatttccaacaggtcgctatatccaccaat
agaagttttcccaaaaaagattctaggaacagttctaccaccagtaatttgttcaaaatagtcacgcaattcattttcgggtttaaa
ttctttaatatcgacaatttcatacgctcctcttttgaaactaaacttatttagaatatccagtgcatttctacaaaaaggacatgt
atacttgacaaaaattgtcactttgttattggccaacctttgttgtacaaattcctcggccattttaatatttaagtgatataaaac
tatctcgacttatttaactctttagtcgagatatatggacgcagatagctatatgatagccaactacagaaggcaaacgctataaaa
aacataattacgacgagcatatttataaatatttttattcagcattacttgatatagtaatattaggcacagtcaaacattcaacca
ctctcgatacattaactctctcattttctttaacaaattctgcaatatcttcgtaaaaagattcttgaaactttttagaatatctat
cgactctagatgaaatagcgttcgtcaacatactatgttttgtatacataaaggcgcccattttaacagtttctagtgacaaaatgc
tagcgatcctaggatcctttagaatcacatagattgacgattcgtctctcttagtaactctagtaaaataatcatacaatctagtac
gcgaaataatattatccttgacttgaggagatctaaacaatctagttttgagaacatcgataagttcatcgggaatgacatacatac
tatctttaatagaactcttttcatccagttgaatggattcgtccttaaccaactgattaatgagatcttctattttatcattttcca
gatgatatgtatgtccattaaagttaaattgtgtagcgcttctttttagtctagcagccaatactttaacatcactaatatcgatat
acaaaggagatgatttatctatggtattaagaattcgtttttcgacatctgtcaaaaccaattcctttttgcctgtatcatccagtt
ttccatcctttgtaaagaaattattttctactagactattaataagactgataaggattcctccataattgcacaatccaaactttt
tcacaaaactagactttacaagatctacaggaatgcgtacttcaggtttcttagcttgtgattttttcttttgtggacattttcttg
tgaccaactcatctaccatttcattgattttagcagtgaaataagctttcaatgcacgggcactgatactattgaaaacgagttgat
cttcaaattccgccatttaagttcaccaaacaacttttaaatacaaatatatcaatagtagtagaataagaactataaaaaaaataa
taattaaccaataccaaccccaacaaccggtattattagttgatgtgactgttttctcatcacttagaacagatttaacaatttcta
taaagtctgtcaaatcatcttccggagaccccataaatacaccaaatatagcggcgtacaacttatccatttatacattgaatattg
gcttttctttatcgctatcttcatcatattcatcatcaatatcaacaagtcccagattacgagccagatcttcttctacattttcag
tcattgatacacgttcactatctccagagagtccgataacgttagccaccacttctctatcaatgattagtttcttgagtgcgaatg
taatttttgtttccgttccggatctatagaaaactacaggtgtgataattgccttggccaattgtctttctcttttactgagtgatt
ctagttcaccttctatagatctgagaatggatgattctccagtcgaaacatattctaccatggctccgtttaatttgttgatgaaga
tggattcatccttaaatgttttctctgtaatagtttccaccgaaagactatgcaaagaatttggaatgcgttccttgtgcttaatgt
ttccatagacggcttctagaagttgatacaacataggactagccgcggtaacttttatttttagaaagtatccatcgcttctatctt
gtttagatttatttttataaagtttagtctctccttccaacataataaaagtggaagtcatttgactagataaactatcagtaagtt
ttatagagatagacgaacaattagcgtattgagaagcatttagtgtaacgtattcgatacattttgcattagatttactaatcgatt
ttgcatactctataacacccgcacaagtctgtagagaatcgctagatgcagtaggtcttggtgaagtttcaactctcttcttgatta
ccttactcatgattaaacctaaataattgtactttgtaatataatgatatatattttcactttatctcatttgagaataaaaatgtt
tttgtttaaccactgcatgatgtacagatttcggaatcacaaaccaccggtggttttattttatccttgtccaatgtgaattgaatg
ggagcggatgcgggtttcgtacgtagatagtacattcccgtttttagaccgagactccatccgtaaaaatgcatactcgttagtttg
gaataactcggatctgctatatggatattcatagattgactttgatcgatgaaggctcccctgtctgcagccatttttatgatcgtc
ttttgtggaatttcccaaatagttttataaactcgcttaatatcttctggaaggtttgtattctgaatggatccaccatctgccata
atcctattcttgatctcatcattccataattttctctcggttaaaactctaaggagatgcggattaactacttgaaattctccagac
aatactctccgagtgtaaatattactggtatacggttccaccgactcattatttcccaaaatttgagcagttgatgcagtcggcata
ggtgccaccaataaactatttctaagaccgtatgttctgattttatcttttagaggttcccaattccaaagatccgacggtacaaca
ttccaaagatcatattgtagaataccgttactggcgtacgatcctacatatgtatcgtatggtccttccttctcagctagttcacaa
ctcgcctctaatgcaccgtaataaatggtttcgaagatcttcttatttagatcttgtgcttccaggctatcaaatggataatttaag
agaataaacgcgtccgctaatccttgaacaccaataccgataggtctatgtctcttattagagatttcagcttctggaataggataa
taattaatatctataattttattgagatttctgacaattactttgaccacatccttcagtttgagaaaatcaaatcgcccatctatt
acaaacatgttcaaggcaacagatgccagattacaaacggctacctcattagcatccgcatattgtattatctcagtgcaaagatta
ctacacttgatagttcctaaattttgttgattactctttttgttacacgcatccttataaagaatgaatggagtaccagtttcaatc
tgagattctataatcgctttccagacgactcgagcctttattatagatttgtatctcctttctctttcgtatagtgtatacaatcgt
tcgaactcgtctccccaaacattgtccaatccaggacattcatccggacacatcaacgaccactctccgtcatccttcactcgtttc
ataaagagatcaggaatccaaagagctataaatagatctctggttctatgttcctcgtttcctgtattctttttaagatcgaggaac
gccataatatcagaatgccacggttccaagtatatggccataactccaggccgtttgtttcctccctgatctatgtatctagcggtg
ttattataaactctcaacattggaataataccgtttgatataccattggtaccggagatatagcttccactggcacgaatattacta
attgatagacctattccccctgccattttagagattaatgcgcatcgttttaacgtgtcatagataccctctatgctatcatcgatc
atgttaagtagaaaacagctagacatttggtgacgactagttcccgcattaaataaggtaggagaagcgtgcgtaaaccatttttca
gaaagtagattgtacgtctcaatagctgagtctatatcccattgatgaattcctactgcgacacgcattaacatgtgctgaggtctt
tcaacgatcttgttgtttattttcaacaagtaggatttttccaaagttttaaaaccaaaatagttgtatgaaaagtctcgttcgtaa
ataataaccgagttgagtttatccttatatttgttaactatatccatggtgatacttgaaataatcggagaatgtttcccattttta
ggattaacatagttgaataaatcctccatcacttcactaaatagtttttttgtttccttgtgtagatttgatacggctattctggcg
gctagaatggcataatccggatgttgtgtagtacaagtggctgctatttcggctgccagagtgtccaattctaccgttgttactcca
ttatatattccttgaataaccttcatagctattttaataggatctatatgatccgtgtttaagccataacataattttctaatacga
gacgtgattttatcaaacatgacattttccttgtatccatttcgtttaatgacaaacatttttgttggtgtaataaaaaaattattt
aacttttcattaatagggatttgacgtacgtagcgtacaaaatgattgttcctggtatatagataaagagtcctatatatttgaaaa
tcgttacggctcgattaaactttaatgattgcatagtgaatatatcattaggatttaactccttgactatcagggcggcaccagaaa
ttaccatcaaaagcattaatacagttatgcctatcgcagttagaacggttatagcatccaccatttatatctaaaaattagatcaaa
gaatatgtgacaaagtcctagttgtatattgagaattgacaaaacaatgtttcttacatattttttttttattagtaaccgacttaa
tagtaggaactggaaaactagacttgattattctataagtatagatacccttccaaataatattctctttgataaaagttccagaaa
atgtagaattttttaaaaagttatcttttgctattaccaagattgtgtttagacgcttattattaatatgagtgatgaaatccacac
cgcctctagatatcgcctttatttccacattagatggtaaatccaatagtgaaactatctttttaggaatgtatggactcgcgttta
gaggagtgaacgtcttgggcgtcggaaaggatgattcgtcaaacgaataaacaatttcacaaatggatgttaatgtattagtaggaa
attttttgacgctagtggaattgaaaattctaatggatgatgttctacctatttcatccgataacatgttaatttccgacaccaacg
gttttaatatttcgatgatatacggtagtctctctttcggacttatatagcttattccacaatacgagtcattatatactccaaaaa
acaaaataactagtataaaatctgtatcgaatgggaaaaacgaaattatcgacataggtatagaatccggaacattgaacgtattaa
tacttaattctttttctgtggtaagtaccgataggttattgacattgtatggttttaaatattctataacttgagacttgatagata
ttagtgatgaattgaaaattatttttatcaccacgtgtgtttcaggatcatcgtcgacgcccgtcaaccaaccgaacggagtaaaat
aaatatcattaatatatgctctagatattagtatttttatcaatcctttgattatcatcttctcgtaggcgaatgattccatgatca
agagtgatttaagaacatcctccggagtattaatgggcttagtaaacagtccatcgttgcaataataaaagttatccaagttaaagg
atattatgcattcgtttaaagatatcacctcatctgacggagacaattttttggtaggttttagagactttgaagctacttgtttaa
caaagttattcatcgtcgtttactattctatttaattttgtagttaatttatcacatatcacattaattgactttttggtccatttt
tccatacgtttatattcttttaatcctgcgttatccgtttccgttatatccagtgatagatcgtgcaggttaaatagaatgctctta
aataatgtcatttttttatccgctaaaaatttaaagaatgtataaacttttttcaaagatttaaaacttttaggtggtgtcctagta
cacaatatcataaacaaactaataaacatcccgcattcagattccaacagctgattaacttccacattaatacagcctattttcgct
ccaaatgtacattcgaaaaatctgaataaaacatcaatgtcgcaatttgtattatccaatacagaatgtttgtgattcgtgttaaaa
ccatcggagaaggaatagaaataaaaattattatagtggtggaattcagttggaatattgcctccggagtcataaaaggatactaaa
cattgttttttatcataaattacacatttccaatgagacaaataacaaaatccaaacattacaaatctagaggtagaacttttaatt
ttgtctttaagtatatacgataagatatgtttattcataaacgcgtcaaatttttcatgaatcgctaaggagtttaagaatctcatg
tcaaattgtcctatataatccacttcggatccataagcaaactgagagactaagttcttaatacttcgattgctcatccaggctcct
ctctcaggctctattttcatcttgacgacctttggattttcaccagtatgtattccttttttttctagacactaaataaaatattta
aaatataatattaatgtactaaaacttatatattattaatttatctaactaaagttagtaaattatatatataattttataattaat
ttaattttactaattttatttagtgtctagaaaaaaa

An example of sVAC fragment 2 (F2, 66679 bp in length) (SEQ ID NO: 17):

tttttttctagacactaaataaaatatttaaaatataatattaatgtactaaaacttatatattattaatttatctaactaaagtta
gtaaattatatatataattttataattaatttaattttactaattttatttagtgtctagaaaaaaaccccctgccattttagagatt
aatgcgcatcgttttaacgtgtcatagataccctctatgctatcatcgatcatgttaagtagaaaacagctagacatttggt
gacgactagttcccgcattaaataaggtaggagaagcgtgcgtaaaccatttttcagaaagtagattgtacgtctcaat
agctgagtctatatcccattgatgaattcctactgcgacacgcattaacatgtgctgaggtctttcaacgatcttgttgtttat
tttcaacaagtaggatttttccaaagttttaaaaccaaaatagttgtatgaaaagtctcgttcgtaaataataaccgagttg
agtttatccttatatttgttaactatatccatggtgatacttgaaataatcggagaatgtttcccatttttaggattaacatagtt
gaataaatcctccatcacttcactaaatagtttttttgtttccttgtgtagatttgatacggctattctggcggctagaatggca
taatccggatgttgtgtagtacaagtggctgctatttcggctgccagagtgtccaattctaccgttgttactccattatatatt
ccttgaataaccttcatagctattttaataggatctatatgatccgtgtttaagccataacataattttctaatacgagacgtg
attttatcaaacatgacattttccttgtatccatttcgtttaatgacaaacatttttgttggtgtaataaaaaaattatttaactttt
cattaatagggatttgacgtacgtagcgtacaaaatgattgttcctggtatatagataaagagtcctatatatttgaaaatc
gttacggctcgattaaactttaatgattgcatagtgaatatatcattaggatttaactccttgactatcagggcggcaccag
aaattaccatcaaaagcattaatacagttatgcctatcgcagttagaacggttatagcatccaccatttatatctaaaaat
tagatcaaagaatatgtgacaaagtcctagttgtatattgagaattgacaaaacaatgtttcttacatattttttttttattagt
aaccgacttaatagtaggaactggaaaactagacttgattattctataagtatagatacccttccaaataatattctctttg
ataaaagttccagaaaatgtagaattttttaaaaagttatcttttgctattaccaagattgtgtttagacgcttattattaatat
gagtgatgaaatccacaccgcctctagatatcgcctttatttccacattagatggtaaatccaatagtgaaactatcttttt
aggaatgtatggactcgcgtttagaggagtgaacgtcttgggcgtcggaaaggatgattcgtcaaacgaataaacaa
tttcacaaatggatgttaatgtattagtaggaaattttttgacgctagtggaattgaaaattctaatggatgatgttctaccta
tttcatccgataacatgttaatttccgacaccaacggttttaatatttcgatgatatacggtagtctctctttcggacttatata
gcttattccacaatacgagtcattatatactccaaaaaacaaaataactagtataaaatctgtatcgaatgggaaaaac
gaaattatcgacataggtatagaatccggaacattgaacgtattaatacttaattctttttctgtggtaagtaccgataggtt
attgacattgtatggttttaaatattctataacttgagacttgatagatattagtgatgaattgaaaattatttttatcaccacgt
gtgtttcaggatcatcgtcgacgcccgtcaaccaaccgaacggagtaaaataaatatcattaatatatgctctagatatt
agtatttttatcaatcctttgattatcatcttctcgtaggcgaatgattccatgatcaagagtgatttaagaacatcctccgga
gtattaatgggcttagtaaacagtccatcgttgcaataataaaagttatccaagttaaaggatattatgcattcgtttaaag
atatcacctcatctgacggagacaattttttggtaggttttagagactttgaagctacttgtttaacaaagttattcatcgtcgt
ttactattctatttaattttgtagttaatttatcacatatcacattaattgactttttggtccatttttccatacgtttatattcttttaatc
ctgcgttatccgtttccgttatatccagtgatagatcgtgcaggttaaatagaatgctcttaaataatgtcatttttttatccgct
aaaaatttaaagaatgtataaacttttttcaaagatttaaaacttttaggtggtgtcctagtacacaatatcataaacaaac
taataaacatcccgcattcagattccaacagctgattaacttccacattaatacagcctattttcgctccaaatgtacattc
gaaaaatctgaataaaacatcaatgtcgcaatttgtattatccaatacagaatgtttgtgattcgtgttaaaaccatcgga
gaaggaatagaaataaaaattattatagtggtggaattcagttggaatattgcctccggagtcataaaaggatactaa
acattgttttttatcataaattacacatttccaatgagacaaataacaaaatccaaacattacaaatctagaggtagaact
tttaattttgtctttaagtatatacgataagatatgtttattcataaacgcgtcaaatttttcatgaatcgctaaggagtttaaga
atctcatgtcaaattgtcctatataatccacttcggatccataagcaaactgagagactaagttcttaatacttcgattgctc
atccaggctcctctctcaggctctattttcatcttgacgacctttggattttcaccagtatgtattcctttacgtgataaatcatc
gattttcaaatccatttgtgagaagtctatcgccttagatactttttcccgtagtcgaggtttaaagaaatacgctaacggta
tactagtaggtaactcaaaaacatcatatatagaatggtaacgcgtctttaactcgtcggttaactctttcttttgatcgagtt
cgtcgctactattgggtctgctcaggtgccccgactctactagttccaacatcataccgataggaatacaagacactttg
ccggcggttgtagatttatcatatttttccactacatatccgttacaatttgttaaaaatttagatacatctatattgctacataa
tccagctagtgaatatatatgacataataaattggtaaatcctagttctggtattttactaattactaaatctgtatatctttcc
atttatcatggaaaagaatttaccagatatcttcttttttccaaactgcgttaatgtattctcttacaaatattcacaagatgaa
ttcagtaatatgagtaaaacggaacgtgatagtttctcattggccgtgtttccagttataaaacatagatggcataacgc
acacgttgtaaaacataaaggaatatacaaagttagtacagaagcacgtggaaaaaaagtatctcctccatcactag
gaaaacccgcacacataaacctaaccgcgaagcaatatatatacagtgaacacacaataagctttgaatgttatagt
tttctaaaatgtataacaaatacagaaatcaattcgttcgatgagtatatattaagaggactattagaagctggtaatagt
ttacagatattttccaattccgtaggtaaacgaacagatactataggtgtactagggaataagtatccatttagcaaaatt
ccattggcctcattaactcctaaagcacaacgagagatattttcagcgtggatttctcatagacctgtagttttaactgga
ggaactggagtgggtaagacgtcacaggtacccaagttattgctttggtttaattatttatttggtggattctctactctagat
aaaatcactaactttcacgaaagaccagtcattctatctcttcctaggatagctttagttagattgcatagcaataccatttt
aaaatcattgggatttaaggtactagatggatctcctatttctttacggtacggatctataccggaagaattaataaacaa
acaaccaaaaaaatatggaattgtattttctacccataagttatctctaacaaaactatttagttatggcactcttattatag
acgaagttcatgagcatgatcaaataggagatattattatagcagtagcgagaaagcatcatacgaaaatagattcta
tgtttttaatgactgccacgttagaggatgaccgagaacggctaaaagtatttttacctaatcccgcatttatacatattcct
ggagatacactgtttaaaattagcgaggtatttattcataataagataaatccatcttccagaatggcatacatagaaga
agaaaagagaaatttagttactgctatacagatgtatactcctcctgatggatcatccggtatagtctttgtggcatccgtt
gcacagtgtcacgaatataaatcatatttagaaaaaagattaccgtatgatatgtatattattcatggtaaggtcttagata
tagacgaaatattagaaaaagtgtattcatcacctaatgtatcgataattatttctactccttatttggaatccagcgttacta
tacgcaatgttacacacatttatgatatgggtaaagtttttgtccccgctccttttggaggatcgcaagaatttatttctaaat
ctatgagagatcaacgaaaaggaagagtaggaagagttaatcctggtacatacgtctatttctatgatctgtcttatatg
aagtctatacagcgaatagattcagaatttctacataattatatattgtacgctaataagtttaatctaacactccccgaag
atttgtttataatccctacaaatttggatattctatggcgtacaaaggaatatatagactcgttcgatattagtacagaaac
atggaataaattattatccaattattatatgaagatgatagagtatgctaaactttatgtactaagtcctattctcgctgagg
agttggataactttgagaggacgggagaattaactagtattgtacgagaagccattttatctctaaatttacgaattaaga
ttttaaattttaaacataaagatgatgatacgtatatacacttttgtaaaatattattcggtgtctataacggaacaaacgct
actatatattatcatagacctctaacgggatatatgaatatgatttcagatactatatttgttcctgtagataataactaaaa
atcaaactctaatgaccacatctttttttagagatgaaaaattttctacatctccttttgtagacacgactaaacattttgcaa
aaaaaagtttattagtgtttagataatcgtatacttcatcagtgtagatagtaaatgtgaacaaataaaaggtattcttact
caatagattggtaaattccatagaatatattaatcctttcttcttgagatcccacatcatttcaaccagagacgttttatcca
atgatttacctcgtactataccacatacaaaactagattttgcagtgacgtcgtacctggtattcctaccaaacaaaatttt
acttttagttcttttagaaaattctaaggtagaatctctatttgccaatatgtcatctatggaattaccactagcaaaaaatg
atagaaatatatattgatacatcgcagctggttttgatctactatactttaaaaacgaatcagattccataattgcctgtata
tcatcagctgaaaaactatgttttacacgtattccttcggcatttctttttaatgatatatcttgtttagacaatgataaagttatc
atgtccatgagagacgcgtctccgtatcgtataaatatttcattagatgttagacgcttcattaggggtatacttctataagg
tttcttaatcagtccatcattggttgcgtcaagaactactatcggatgttgttgggtatctctagtgttacacatggccttacta
aagtttgggtaaataactatgatatctctattaattatagatgcatatatttcattcgtcaaggatattagtatcgacttgctat
cgtcattaatacgtgtaatgtaatcatataaatcatgcgatagccaaggaaaattcaaatagatgttcatcatataatcgt
cgctataattcatattaatacgttgacattgactaatttgtaatatagcctcgccacgaagaaagctctcgtattcagtttca
tcgataaaggataccgttaaatataactggttgccgatagtctcatagtctattaagtggtaagtttcgtacaaatacaga
atccctaaaatattatctaatgttggattaatctttaccataactgtataaaatggagacggagtcataactattttaccgttt
gtacttactggaatagacgaaggaataatctccggacatgctggtaaagacccaaatgtctgtttgaagaaatccaat
gttccaggtcctaatctcttaacaaaaattacgatattcgatcccgatatcctttgcattctatttaccagcatatcacgaac
tatattaagattatctatcatgtctattctcccaccgttatataaatcgcctccgctaagaaacgttagtatatccatacaatg
gaatacttcatttctaaaatagtattcgttttctaattctttaatgtgaaatcgtatactagaaagggaaaaattatctttgagtt
ttccgttagaaaagaaccacgaaactaatgttctgattgcgtccgattccgttgctgaattaatggatttacaccaaaaa
ctcatataacttctagatgtagaagcattcgctaaaaaattagtagaatcaaaggatataagtagatgttccaacaagt
gagcaattcccaagatttcatctatatcattctcgaatccgaaattagaaattcccaagtagatatcctttttcatccgatcg
ttgatgaaaatacgaactttattcggtaagacaatcatttactaaggagtaaaataggaagtaatgttcgtatgtcgttatc
atcgtataaattaaaggtgtgttttttaccattaagtgacattataattttaccaatattggaattataatataggtgtatttgcg
cactcgcgacggttgatgcatcggtaaatatagctgtatctaatgttctagtcggtatttcatcatttcgctgtctaataatag
cgttttctctatctgtttccattacagctgcctgaagtttattggtcggataatatgtaaaataataagaaatacatacgaat
aacaaaaataaaataagatataataaagatgccatttagagatctaattttgttcaacttgtccaaattcctacttacaga
agatgaggaatcgttggagatagtgtcttccttatgtagaggatttgaaatatcttatgatgacttgataacttactttccag
ataggaaataccataaatatatttctaaagtatttgaacatgtagatttatcggaggaattaagtatggaattccatgata
caactttgcgagatttagtctatcttagattgtacaagtattccaagtgtatacggccgtgttataaattaggagataatcta
aaaggcatagttgttataaaggacaggaatatttatattagggaagcaaatgatgacttgatagaatatctcctcaagg
aatacactcctcagatttatacatattctaatgagcgcgtccccataactggttcaaaattaattctttgtggattttctcaag
ttacatttatggcgtatacaacgtcgcatataacaacaaataaaaaggtagatgttctcgtttccaaaaaatgtatagat
gaactagtcgatccaataaattatcaaatacttcaaaatttatttgataaaggaagcggaacaataaacaaaatactc
aggaagatattttattcggtaacaggtggccaaactccataggtagctttttctatttcggattttagaatttccaaattcacc
agcgatttatctgttttggtgaaatccaaggatttattaatgtccacaaatgccatttgttttgtctgtggattgtatttgaaaat
ggaaacgatgtagttagatagatgcgctgcgaagtttcctattagggttccgcgcttcacgtcacccagcatacttgaat
caccatcctttaaaaaaaatgataagatatcaacatggagtatatcatactcggattttaattcttctactgcatcactgac
attttcacaaatactacaatacggtttaccgaaaataatcagtacgttcttcatttatgggtatcaaaaacttaaaatcgtta
ctgctggaaaataaatcactgacgatattagatgataatttatacaaagtatacaatggaatatttgtggatacaatgag
tatttatatagccgtcgccaattgtgtcagaaacttagaagagttaactacggtattcataaaatacgtaaacggatggg
taaaaaagggagggcatgtaaccctttttatcgatagaggaagtataaaaattaaacaagacgttagagacaagag
acgtaaatattctaaattaaccaaggacagaaaaatgctagaattagaaaagtgtacatccgaaatacaaaatgtta
ccggatttatggaagaagaaataaaggcagaaatgcaattaaaaatcgataaactcacatttcaaatatatttatctga
ttctgataacataaaaatatcattgaatgagatactaacacatttcaacaataatgagaatgttacattattttattgtgatg
aacgagacgcagaattcgttatgtgtctcgaggctaaaacacatttctctaccacaggagaatggccgttgataataa
gtaccgatcaggatactatgctatttgcatctgctgataatcatcctaagatgataaaaaacttaactcaactgtttaaata
tgttccatctgcagaggataactatttagcaaaattaacggcgttagtgaatggatgtgatttctttcctggactctatggg
gcatctataacacccaacaacttaaacaaaatacaattgtttagtgattttacaatcgataatatagtcactagtttggca
attaaaaattattatagaaagactaactctaccgtagacgtgcgtaatattgttacgtttataaacgattacgctaatttag
acgatgtctactcgtatattcctccttgtcaatgcactgttcaagaatttatattttccgcattagatgaaaaatggaatgaat
ttaaatcatcttatttagaaagcgtgccgttaccctgccaattaatgtatgcgttagaaccacgcaaggagattgatgtttc
agaagttaaaactttatcatcttatatagatttcgaaaatactaaatcagatatcgatgttataaaatctatatcctcgatctt
cggatattctaacgaaaactgtaacacgatagtattcggcatctataaggataatttactactgagtataaatagttcattt
tactttaacgatagtctgttaataaccaatactaaaagtgataatataataaatataggttactagattaaaaatggtgttc
caactcgtgtgctctacgtgcggcaaagatatttctcacgaacgatataaattgattatacgaaaaaaatcattaaagg
atgtactcgtcagtgtaaagaacgaatgttgtaggttaaaattatctacacaaatagaacctcaacgtaacttaacagt
gcaacctctattggatataaactaatatggatccggttaattttatcaagacatatgcgcctagaggttctattatttttatta
attataccatgtcattaacaagtcatttgaatccatcgatagaaaaacatgtgggtatttattatggtacgttattatcggaa
cacttggtagttgaatcaacatatagaaaaggagttcgaatagtcccattggatagtttttttgaaggatatcttagtgcaa
aagtatacatgttagagaatattcaagttatgaaaatagcagctgatacgtcattaactttattgggtattccgtatggattt
ggtcatgatagaatgtattgttttaaattggtagctgactgttataaaaatgccggtattgatacatcgtctaaacgaatatt
aggtaaagatatttttctgagccaaaacttcacagacgataatagatggataaagatatatgattctaataatttaacattt
tggcaaattgattaccttaaagggtgagttaatatgcataactactcctccgttgttttttccctcgttctttttcttaacgttgttt
gccatcactctcataatgtaaagatattctaaaatggtaaacttttgcatatcggacgcagaaattggtataaatgttgta
attgtattatttcccgtcaatggactagtcacagctccatcagttttatatcctttagagtatttctcactcgtgtctaacattcta
gagcattccatgatctgtttatcgttgatattggccggaaagatagattttttattttttattatattactattggcaattgtagata
taacttctggtaaatatttttctaccttttcaatctcttctattttcaagccggctatatattctgctatattgttgctagtatcaata
ccttttctggctaagaagtcatatgtggtattcactatatcagttttaactggtagttccattagcctttccacttctgcagaat
aatcagaaattggttctttaccagaaaatccagctactataataggctcaccgatgatcattggcaaaatcctatattgta
ccagattaatgagagcatatttcatttccaataattctgctagttcttgagacattgatttatttgatgaatctagttggttctct
agatactctaccatttctgccgcatacaataacttgttagataaaatcagggttatcaaagtgtttagcgtggctagaata
gtgggcttgcatgtattaaagaatgcggtagtatgagtaaaccgttttaacgaattatatagtctccagaaatctgtggcg
ttacatacatgagccgaatgacatcgaagattgtccaatatttttaatagctgctctttgtccattatttctatatttgactcgc
aacaattgtagataccattaatcaccgattcctttttcgatgccggacaatagcacaattgtttagctttggactctatgtatt
cagaattaatagatatatctcttaatacagattgcactatacattttgaaactatgtcaaaaattgtagaacgacgctgttc
tgcagccatttaactttaaataatttacaaaaatttaaaatgagcatccgtataaaaatcgataaactgcgccaaattgt
ggcatatttttcagagttcagtgaagaagtatctataaatgtagactcgacggatgagttaatgtatatttttgccgccttgg
gcggatctgtaaacatttgggccattatacctctcagtgcatcagtgttctaccgcggagccgaaaatattgtgtttaatct
tcctgtgtccaaggtaaaatcgtgtttgtgtagttttcacaatgatgccatcatagatatagaacctgatctggaaaataat
ctagtaaaactttctagttatcatgtagtaagtgtcgattgtaataaggaactgatgcctattaggacagatactactatttg
tctaagtatagatcaaaagaaatcttacgtgtttaattttcacaagtatgaagaaaaatgttgtggtagaaccgtcattcat
ttagaatggttgttgggctttatcaagtgtattagtcagcatcagcatttggctattatgtttaaagatgacaatattattatga
agactcctggtaatactgatgcattttccagggaatattctatgactgaatgttctcaagaactacaaaagttttctttcaaa
atagctatctcgtctctcaacaaactacgaggattcaaaaagagagtcaatgtttttgaaactagaatcgtaatggata
atgacgataacattttaggaatgttgttttcggatagagttcaatcctttaagatcaacatctttatggcgtttttagattaata
ctttcaatgagataaatatgggtggcggagtaagtgttgagctccctaaacgggatccgcctccgggagtacccactg
atgagatgttattaaacgtggataaaatgcatgacgtgatagctcccgctaagcttttagaatatgtgcatataggacca
ctagcaaaagataaagaggataaagtaaagaaaagatatccagagtttagattagtcaacacaggacccggtggt
ctttcggcattgttaagacaatcgtataatggaaccgcacccaattgctgtcgcacttttaatcgtactcattattggaaga
aggatggaaagatatcagataagtatgaagagggtgcagtattagaatcgtgttggccagacgttcacgacaccgg
aaaatgcgatgttgatttattcgactggtgtcagggggatacgttcgatagaaacatatgccatcagtggatcggttcag
cctttaataggagtaatagaactgtagagggtcaacaatcgttaataaatctgtataataagatgcaaacattatgtagt
aaagatgctagtgtaccaatatgtgaatcatttttgcatcatttacgcgcacacaatacagaagatagcaaagagatg
atcgattatattctaagacaacagtctgcggactttaaacagaaatatatgagatgtagttatcccactagagataagtt
agaagagtcattaaaatatgcggaacctcgagaatgttgggatccagagtgttcgaatgccaatgttaatttcttgctaa
cacgtaattataataatttaggactttgcaatattgtacgatgtaatactagcgtgaacaacttacagatggataaaactt
cctcattaagattgtcatgtggattaagcaatagtgatagattttctactgttcccgtcaatagagcaaaagtagttcaaca
taatattaaacactcgttcgacctaaaattgcatttgatcagtttattatctctcttggtaatatggatactaattgtagctattt
aaatgggtgccgcggcaagcatacagacgacggtgaatacactcagcgaacgtatctcgtctaaattagaacaag
aagcgaatgctagtgctcaaacaaaatgtgatatagaaatcggaaatttttatatccgacaaaaccatggatgtaacc
tcactgttaaaaatatgtgctctgcggacgcggatgctcagttggatgctgtgttatcagccgctacagaaacatatagt
ggattaacaccggaacaaaaagcatacgtgccagctatgtttactgctgcgttaaacattcagacgagtgtaaacact
gttgttagagattttgaaaattatgtgaaacagacttgtaattctagcgcggtcgtcgataacaaattaaagatacaaaa
cgtaatcatagatgaatgttacggagccccaggatctccaacaaatttggaatttattaatacaggatctagcaaagga
aattgtgccattaaggcgttgatgcaattgacgactaaggccactactcaaatagcacctaaacaagttgctggtaca
ggagttcagttttatatgattgttatcggtgttataatattggcagcgttgtttatgtactatgccaagcgtatgttgttcacatc
caccaatgataaaatcaaacttattttagccaataaggaaaacgtccattggactacttacatggacacattctttagaa
cttctccgatggttattgctaccacggatatgcaaaactgaaaatatattgataatattttaatagattaacatggaagttat
cactgatcgtctagacgatatagtgaaacaaaatatagcggatgaaaaatttgtagattttgttatacacggtctagagc
atcaatgtcctgctatacttcgaccattaattaggttgtttattgatatactattatttgttatagtaatttatatttttacggtacgt
ctagtaagtagaaattatcaaatgttgttggcgttggtggcgctagtcatcacattaactattttttattactttatactataata
gtactagactgacttctaacaaacatctcacctgccataaataaatgcttgatattaaagtcttctatttctaacactattcc
atctgtggaaaataatactctgacattatcgctaattgacacatcggtgagtgatatgcctataaagtaataatcttctttg
ggcacatataccagtgtaccaggttctaacaacctatttactggtgctcctatagcatactttttctttaccttgagaatatcc
atcgtttgcttggtcaatagcgatatgtgattttttatcaaccactcgaaaaagtaattggagtgttcatatcctctacgggct
attgtctcatggccgtgtatgaaatttaagtaacacgactgtggtagatttgttctatagagccggttgccgcaaatagat
agaactaccaatatgtctgtacaaatgttaaacattaattgattaacagaaaaaacaatgttcgttctgggaatagaaa
ccagatcaaaacaaaattcgttagaatatatgccacgtttatacattgaatataaaataactacagtttgaaaaataac
agtatcatttaaacatttaacttgcggggttaatctcacaactttactgtttttgaactgttcaaaatatagcatagatccgtg
agaaatacgtttagccgcctttaatagaggaaatcccaccgcctttctggatctcaccaacgacgatagttctgaccag
caactcatttcttcatcatccacctgttttaacatataataggcaggagatagatatccgtcattgcaatattccttctcgtag
gcacacaatctaatattgataaaatctccattctcttctctgcatttattatcttgtttcggtggctgattaggctgtagtcttggt
ttaggctttggtatatcgttgttgaatctattttggtcattaaatctttcatttcttcctggtatatttttatcacctcgtttggttggatt
tttgtctatattatcgtttgtaacatcggtacgggtattcatttatcacaaaaaaaacttctctaaatgagtctactgctagaa
aacctcatcgaagaagataccatattttttgcaggaagtatatctgagtatgatgatttacaaatggttattgccggcgca
aaatccaaatttccaagatctatgctttctatttttaatatagtacctagaacgatgtcaaaatatgagttggagttgattcat
aacgaaaatatcacaggagcaatgtttaccacaatgtataatataagaaacaatttgggtctaggagatgataaacta
actattgaagccattgaaaactatttcttggatcctaacaatgaagttatgcctcttattattaataatacggatatgactgc
cgtcattcctaaaaaaagtggtaggagaaagaataagaacatggttatcttccgtcaaggatcatcacctatcttgtgta
ttttcgaaactcgtaaaaagattaatatttataaagaaaatatggaatccgcgtcgactgagtatacacctatcggaga
caacaaggctttgatatctaaatatgcgggaattaatatcctaaatgtgtattctccttccacatccataagattgaatgcc
atttacggattcaccaataaaaataaactagagaaacttagtactaataaggaactagaatcgtatagttctagccctct
tcaagaacccattaggttaaatgattttctgggactattggaatgtgttaaaaagaatattcctctaacagatattccgac
aaaggattgattactataaatggagaatgttcctaatgtatactttaatcctgtgtttatagagcccacgtttaaacattcttt
attaagtgtttataaacacagattaatagttttatttgaagtattcgttgtattcattctaatatatgtattttttagatctgaattaa
atatgttcttcatgcctaaacgaaaaatacccgatcctattgatagattacgacgtgctaatctagcgtgtgaagacgat
aaattaatgatctatggattaccatggatgacaactcaaacatctgcgttatcaataaatagtaaaccgatagtgtataa
agattgtgcaaagcttttgcgatcaataaatggatcacaaccagtatctcttaacgatgttcttcgcagatgatgattcattt
tttaagtatttggctagtcaagatgatgaatcttcattatctgatatattgcaaatcactcaatatctagactttctgttattatta
ttgatccaatcaaaaaataaattagaagccgtgggtcattgttatgaatctctttcagaggaatacagacaattgacaa
aattcacagactttcaagattttaaaaaactgtttaacaaggtccctattgttacagatggaagggtcaaacttaataaa
ggatatttgttcgactttgtgattagtttgatgcgattcaaaaaagaatcctctctagctaccaccgcaatagatcctgttag
atacatagatcctcgtcgcaatatcgcattttctaacgtgatggatatattaaagtcgaataaagtgaacaataattaatt
ctttattgtcatcatgaacggcggacatattcagttgataatcggccccatgttttcaggtaaaagtacagaattaattaga
cgagttagacgttatcaaatagctcaatataaatgcgtgactataaaatattctaacgataatagatacggaacggga
ctatggacgcatgataagaataattttgaagcattggaagcaactaaactatgtgatgtcttggaatcaattacagatttc
tccgtgataggtatcgatgaaggacagttctttccagacattgttgaattctgtgagcgtatggcaaacgaaggaaaaa
tagttatagtagccgcactcgatgggacatttcaacgtaaaccgtttaataatattttgaatcttattccattatctgaaatgg
tggtaaaactaactgctgtgtgtatgaaatgctttaaggaggcttccttttctaaacgattgggtgaggaaaccgagata
gaaataataggaggtaatgatatgtatcaatcggtgtgtagaaagtgttacatcgactcataatattatattttttatctaaa
aaactaaaaataaacattgattaaattttaatataatacttaaaaatggatgttgtgtcgttagataaaccgtttatgtatttt
gaggaaattgataatgagttagattacgaaccagaaagtgcaaatgaggtcgcaaaaaaactgccgtatcaagga
cagttaaaactattactaggagaattattttttcttagtaagttacagcgacacggtatattagatggtgccaccgtagtgt
atataggatctgctcccggtacacatatacgttatttgagagatcatttctataatttaggagtgatcatcaaatggatgct
aattgacggccgccatcatgatcctattttaaatggattgcgtgatgtgactctagtgactcggttcgttgatgaggaatat
ctacgatccatcaaaaaacaactgcatccttctaagattattttaatttctgatgtgagatccaaacgaggaggaaatga
acctagtacggcggatttactaagtaattacgctctacaaaatgtcatgattagtattttaaaccccgtggcgtctagtctt
aaatggagatgcccgtttccagatcaatggatcaaggacttttatatcccacacggtaataaaatgttacaaccttttgct
ccttcatattcagctgaaatgagattattaagtatttataccggtgagaacatgagactgactcgagttaccaaatcaga
cgctgtaaattatgaaaaaaagatgtactaccttaataagatcgtccgtaacaaagtagttgttaactttgattatcctaat
caggaatatgactattttcacatgtactttatgctgaggaccgtgtactgcaataaaacatttcctactactaaagcaaag
gtactatttctacaacaatctatatttcgtttcttaaatattccaacaacatcaactgaaaaagttagtcatgaaccaatac
aacgtaaaatatctagcaaaaattctatgtctaaaaacagaaatagcaagagatccgtacgcagtaataaatagaa
acgtactactgagatatactaccgatatagagtataatgatttagttactttaataaccgttagacataaaattgattctatg
aaaactgtgtttcaggtatttaacgaatcatccataaattatactccggttgatgatgattatggagaaccaatcattataa
catcgtatcttcaaaaaggtcataacaagtttcctgtaaattttctatacatagatgtggtaatatctgacttatttcctagcttt
gttagactagatactacagaaactaatatagttaatagtgtactacaaacaggcgatggtaaaaagactcttcgtcttcc
caaaatgttagagacggaaatagttgtcaagattctctatcgccctaatataccattaaaaattgttagatttttccgcaat
aacatggtaactggagtagagatagccgatagatctgttatttcagtcgctgattaatcaattagtagagatgagataag
aacattataataatcaataatatatcttatatctcgtttagaaaaatgctaatattaaaatagctaacgctagtaatccaat
cggaagccatttgatatctataatagggtatctaatttcctgattcagatagcggacagctatattctcggtagctactcgtt
tggaatcacaaacattatttacatctaatttactatctgtaatggaaacgtttcccaatgaaatggtacaatccgatacatt
gcattttgttatatttttttttaaagaggctggtaacaacgcatcgcttcgtttacatggctcgtaccaacaataatagggtaa
tcttgtatctattcctatccgtactatgcttttatcaggataaatacatttacatcgtatatcgtctttgttagcatcacagaatg
cataaatttgttcgtccgtcatgataaaaatttaaagtgtaaatataactattatttttatagttgtaataaaaagggaaattt
gattgtataccttcggttctttaaaagaaactgacttgataaaaatggctgtaatctctaaggttacgtatagtctatatgat
caaaaagagattaatgctacagatattatcattagtcatgttaaaaatgacgacgatatcggtaccgttaaagatggta
gactaggtgctatggatggggcattatgtaagacttgtgggaaaacggaattggaatgtttcggtcactggggtaaagt
aagtatttataaaactcatatagttaagcctgaatttatttcagaaattattcgtttactgaatcatatatgtattcactgcgga
ttattgcgttcacgagaaccgtattccgacgatattaacctaaaagagttatcgggacacgctcttaggagattaaagg
ataaaatattatccaagaaaaagtcatgttggaacagtgaatgtatgcaaccgtatcaaaaaattactttttcaaagaa
aaaggtttgtttcgtcaacaagttggatgatattaacgttcctaattctctcatctatcaaaagttaatttctattcatgaaaa
gttttggccattattagaaattcatcaatatccagctaacttattttatacagactactttcccatccctccgttgattattagac
cggctattagtttttggatagatagtatacccaaagaaaccaatgaattaacttacttattaggtatgatcgttaagaattgt
aacttgaatgctgatgaacaggttatccagaaggcggtaatagaatacgatgatattaaaattatttctaataacacttc
cagtatcaatttatcatatattacatccggcaaaaataatatgattagaagttatatcgtcgcccgacgaaaagatcaga
cggctagatctgtaattggtcccagtacatctatcaccgttaatgaggtaggaatgcccgcatatattagaaatacactt
acagaaaagatatttgttaatgcctttacagtggataaagttaaacaactattagcatcaaaccaagttaaattttacttta
ataaacgattaaaccaattaacaagaatacgccaaggaaagtttatcaaaaataaaatacatttattgcctggtgattg
ggtagaagtagctgttcaagaatatacaagtattatttttggaagacagccgtctctacatagatacaacgtcatcgcttc
atctatcagagctaccgaaggagatactatcaaaatatctcccggaattgccaactctcaaaatgctgatttcgacgga
gatgaagaatggatgatattagaacaaaatcctaaagctgtaattgaacaaagtattcttatgtatccgacgacgttact
caaacacgatattcatggagcccccgtttatggatctattcaagatgaaatcgtagcagcgtattcattgtttaggataca
agatctttgtttagatgaagtattgaacatcttggggaaatatggaagagagttcgatcctaaaggtaaatgtaaattca
gcggtaaagatatctatacttacttgataggtgaaaagattaattatccgggtctcttaaaggatggtgaaattattgcaa
acgacgtagatagtaattttgttgtggctatgaggcatctgtcattggctggactcttatccgatcataagtcgaacgtgga
aggtatcaactttattatcaagtcatcttatgtttttaagagatatctatctatttacggttttggggtgacattcaaagatctga
gaccaaattcgacgttcactaataaattggaggccatcaacgtagaaaaaatagaacttatcaaagaagcatacgc
caaatatctcaacgatgtaagagacgggaaaatagttccattatctaaagctttagaggcggactatgtggaatccat
gttatccaacttgacaaatcttaatatccgagagatagaagaacatatgagacaaacgctgatagatgatccagata
ataacctcctgaaaatggccaaagcgggttataaagtaaatcctacagaactaatgtatattctaggtacgtatggaca
acaaaggattgatggtgaaccagcagagactcgagtattgggtagagtcttaccttactatcttccagactctaaggat
ccagaaggaagaggttacattcttaattctttaacaaaaggattaacaggttctcaatattacttttcgatgctggttgcaa
gatctcaatctactgatatcgtctgtgaaacatcacgtaccggaacactggctagaaaaatcattaaaaagatggagg
atatggtggtcgacggatacggacaagtagttataggtaatacgctcatcaagtacgccgccaattataccaaaattct
aggctcagtatgtaaacctgtagatcttatctatccagatgagtccatgacttggtatttggaaattagtgctctgtggaat
aaaataaaacagggattcgtttactctcagaaacagaaacttgcaaagaagacattggcgccgtttaatttcctagtatt
cgtcaaacccaccactgaggataatgctattaaggttaaggatctgtacgatatgattcataacgtcattgatgatgtga
gagagaaatacttctttacggtatctaatatagattttatggagtatatattcttgacgcatcttaatccttctagaattagaat
tacaaaagaaacggctatcactatctttgaaaagttctatgaaaaactcaattatactctaggtggtggaactcctattgg
aattatttctgcacaggtattgtctgagaagtttacacaacaagccctgtccagttttcacactactgaaaaaagtggtgc
cgtcaaacaaaaacttggtttcaacgagtttaataacttgactaatttgagtaagaataagaccgaaattatcactctgg
tatccgatgatatctctaaacttcaatctgttaagattaatttcgaatttgtatgtttgggagaattaaatccagacatcactct
tcgaaaagaaacagataggtatgtagtagatataatagtcaatagattatacatcaagagagcagaaattaccgaat
tagtcgtcgaatatatgattgaacgattcatctcctttagcgtcattgtaaaggaatggggtatggaaacattcattgagg
acgaggataatattagatttactgtctatctaaatttcgttgaaccagaggaattgaatcttagtaagtttatgatggttcttc
cgggggcagccaacaagggaaagattagtaaattcaagattcctatctctgattatacgggttatgacgacttcaatca
aacaaaaaagctcaataagatgactgtagaactcatgaatctaaaagaattaggttctttcgatttggaaaacgtcaa
cgtgtatcctggagtatggaatacatacgatatcttcggtatcgaggccgctcgtgaatacttgtgcgaagccatgttaa
acacctatggagaagggttcgattatctgtatcagccttgtgatcttctcgctagtttactatgtgctagttacgaaccaga
atcagtgaataaattcaagttcggcgcagctagtactcttaagagagctacgttcggagacaataaagcattgttaaac
gcggctcttcataaaaagtcagaacctattaacgataatagtagctgccacttttttagcaaggtccctaatataggaac
tggatattacaaatactttatcgacttgggtcttctcatgagaatggaaaggaaactatctgataagatatcttctcaaaa
gatcaaggaaatggaagaaacagaagacttttaattcttatcaataacatatttttctatgatctgtcttttaaacgatggat
tttccacaaatgcgcctctcaagtccctcatagaatgatacacgtataaaaaatatagcataggcaatgactccttattttt
agacattagatatgccaaaatcatagccccgcttctatttactcccgcagcacaatgaaccaacacgggctcgtttcgtt
gatcacatttagataaaaaggcggttacgtcgtcaaaatatttactaatatcggtagttgtatcatctaccaacggtatat
gaataatattaatattagagttaggtaatgtatatttatccatcgtcaaatttaaaacatatttgaacttaacttcagatgatg
gtgcatccatagcatttttataatttcccaaatacacattattggttacccttgtcattatagtgggagatttggctctgtgcat
atctccagttgaacgtagtagtaagtatttatacaaacttttcttatccatttataacgtacaaatggataaaactactttatc
ggtaaacgcgtgtaatttagaatacgttagagaaaaggctatagtaggcgtacaagcagccaaaacatcaacactt
atattctttgttattatattggcaattagtgcgctattactctggtttcagacgtctgataatccagtctttaatgaattaacgag
atatatgcgaattaaaaatacggttaacgattggaaatcattaacggatagcaaaacaaaattagaaagtgatagag
gtagacttctagccgctggtaaggatgatatattcgaattcaaatgtgtggatttcggcgcctattttatagctatgcgattg
gataagaaaacatatctgccgcaagctattaggcgaggtactggagacgcgtggatggttaaaaaggcggcaaag
gtcgatccatctgctcaacaattttgtcagtatttgataaaacacaagtctaataatgttattacttgtggtaatgagatgtta
aatgaattaggttatagcggttattttatgtcaccgcattggtgttccgattttagtaatatggaatagtgttagataaatgcg
gtaacgaatgttcctgtaaggaaccataacagtttagatttaacgttaaagatgagcataaacataataaacaaaatta
caatcaaacctataacattaatatcaaacaatccaaaaaatgaaatcagtggagtagtaaacgcgtacataactcct
ggataacgtttagtagctgccgttcctattctagaccaaaaattcggtttcatgttttcgaaacggtgttctgcaacaagtc
ggggatcgtgttctacatatttggcggcattatccagtatctgcctattgatcttcatttcgttttcaattctggctatttcaaaat
aaaatcccgatgatagacctccagactttataatttcatctacgatgttcagcgccgtagtaactctaataatataggctg
ataagctaacatcataccctcctgtatatgtgaatatggcatgatttttgtccattacaagctcggttttaactttattgcctgt
aataatttctctcatctgtaggatatctatttttttgtcatgcattgccttcaagacgggacgaagaaacgtaatatcctcaat
aacgttatcgttttctacaataactacatattctacctttttattttctaactcggtaaaaaaattagaatcccatagggctaa
atgtctagcgatatttcttttcgtttcctctgtacacatagtgttacaaaaccctgaaaagaagtgagtatacttgtcatcattt
ctaatgtttcctccagtccactgtataaacgcataatccttgtaatgatctggatcatccttgactaccacaacatttctttttt
ctggcataacttcgttgtcctttacatcatcgaacttctgatcattaatatgctcatgaacattaggaaatgtttctgatggaa
gtctatcaataactggcacaacaataacaggagttttcgccgccgccatttagttattgaaattaatcatatacaactcttt
aatacgagttatattttcgtctatccattgtttcacatttacatatttcgacaaaaagatataaaatgcgtattccaatgcttct
ctgtttaatgaattactaaaatatacaaacacgtcactgtctggcaataaatgatatcttagaatattgtaacaatttattttg
tattgcacatgttcgtgatctatgagttcttcttcgaatggcataggatctccgaatctgaaaacgtataaataggagttag
aataataatatttgagagtattggtaatatataaactctttagcggtataattagtttttttctctcaatttctatttttagatgtgat
ggaaaaatgactaattttgtagcattagtatcatgaactctaatcaaaatcttaatatcttcgtcacacgttagctctttgaa
gtttttaagagatgcatcagttggttcgaccgatggagtaggtgcaacaattttttgttcgatgtatgtatgtactggagcca
ttgttttaactataatggtgcttgtatcgaaaaactttaatgcagatagcggaagctcttcgccgcgactttctacatcgtaa
ttgggttctaacgccgatctctgaatggatactagttttctaagttctaatgtgattctctgaaaatgtaaatccaattcctcc
ggcattatagatgtgtatacatcggtaaataaaactatagtatccaacgatcccttctcgcaaattctagtcttaaccaaa
aaatcgtatataaccacggagatggcgtatttaagagtggattcttctaccgttttgttcttggatgtcatataggaaactat
aaagtccgcactactgttaagaatgattactaacgcaactatatagttcaaattaagcattttggaaacataaaataact
ctgtagacgatacttgactttcgaataagtttgcagacaaacgaagaaagaacagacctctcttaatttcagaagaaa
actttttttcgtattcctgacgtctagagtttatatcaataagaaagttaagaattagtcggttaatgttgtatttcattacccaa
gtttgagatttcataatattatcaaaagacatgataatattaaagataaagcgctgactatgaacgaaatagctatatgg
ttcgctcaaaaatatagtcttgttaaacgtggaaacgataactgtatttttaatcacgtcagcggcatctaaattaaatata
ggtatatttattccacacactctacaatatgccacaccatcttcataataaataaattcgttagcaaaattattaattttagtg
aaatagttagcgtcaactttcatagcttccttcaatctaatttgatgctcacacggtgcgaattccactctaacatcccttttc
catgcctcaggttcatcgatctctataatatctagttttttgcgtttcacaaacacaggctcgtctctcgcgatgagatctgta
tagtaactatgtaaatgataactagatagaaagatgtagctatatagatgacgatcctttaagagaggtataataacttt
accccaatcagatagactgttgttatggtcttcggaaaaagaatttttataaatttttccagtattttccaaatatacgtactta
acatctaaaaaatccttaatgataataggaatggataatccgtctattttataaagaaatacatatcgcacattatacttttt
tttggaaatgggaataccgatgtgtctacataaatatgcaaagtctaaatattttttagagaatcttaattggtccaaattctt
ttccaagtacggtaatagatttttcatattgaacggtatcttcttaatctctggttctagttccgcattaaatgatgaaactaag
tcactatttttataactaacgattacatcacctctaacatcatcatttaccagaatactgatcttcttttgtcgtaaatacatgtc
taatgtgttaaaaaaaagatcatacaagttatacgtcatttcatctgtggtattcttgtcattgaaggataaactcgtactaa
tctcttctttaacagcctgttcaaatttatatcctatatacgaaaaaatagcaaccagtgtttgatcatccgcgtcaatattct
gttctatcgtagtgtataacaatcgtatatcttcttctgtgatagtcgatacgttataaaggttgataacgaaaatatttttattt
cgtgaaataaagtcatcgtaggattttggacttatattcgcgtctagtagatatgcttttatttttggaatgatctcaattagaa
tagtctctttagagtccatttaaagttacaaacaactaggaaattggtttatgatgtataatttttttagtttttatagattctttatt
ctatacttaaaaaatgaaaataaatacaaaggttcttgagggttgtgttaaattgaaagcgagaaataatcataaattat
ttcattatcgcgatatccgttaagtttgtatcgtaatggcgtggtcaattacaaataaagcggatactagtagcttcacaaa
gatggctgaaatcagagctcatctaaaaaatagcgctgaaaataaagataaaaacgaggatattttcccggaagat
gtaataattccatctactaagcccaaaaccaaacgagccactactcctcgtaaaccagcggctactaaaagatcaac
caaaaaggaggaagtggaagaagaagtagttatagaggaatatcatcaaacaactgaaaaaaattctccatctcct
ggagtcagcgacattgtagaaagcgtggccgctgtagagctcgatgatagcgacggggatgatgaacctatggtac
aagttgaagctggtaaagtaaatcatagtgctagaagcgatctctctgacctaaaggtggctaccgacaatatcgttaa
agatcttaagaaaattattactagaatctctgcagtatcgacggttctagaggatgttcaagcagctggtatctctagaca
atttacttctatgactaaagctattacaacactatctgatctagtcaccgagggaaaatctaaagttgttcgtaaaaaagtt
aaaacttgtaagaagtaaatgcgtgcacttttttataaagatggtaaactctttaccgataataattttttaaatcctgtatca
gacgataatccagcgtatgaggttttgcaacatgttaaaattcctactcatttaacagatgtagtagtatatgaacaaac
gtgggaggaggcgttaactagattaatttttgtgggaagtgattcaaaaggacgtagacaatacttttacggaaaaatg
catgtacagaatcgcaacgctaaaagagatcgtatttttgttagagtatataacgttatgaaacgaattaattgttttataa
acaaaaatataaagaaatcgtccacagattccaattatcagttggcggtttttatgttaatggaaactatgttttttattagat
ttggtaaaatgaaatatcttaaggagaatgaaacagtagggttattaacactaaaaaataaacacatagaaataagt
cccgatgaaatagttatcaagtttgtaggaaaggacaaagtttcacatgaatttgttgttcataagtctaatagactatata
agccgctattgaaactgacggatgattctagtcccgaagaatttctgttcaacaaactaagtgaacgaaaggtatatga
atgtatcaaacagtttggtattagaatcaaggatctccgaacgtatggagtcaattatacgtttttatataatttttggacaa
atgtaaagtccatatctcctcttccatcaccaaaaaagttaatagcgttaactatcaaacaaactgctgaagtggtaggt
catactccatcaatttcaaaaagagcttatatggcaacgactattttagaaatggtaaaggataaaaattttttagatgta
gtatctaaaactacgttcgatgaattcctatctatagtcgtagatcacgttaaatcatctacggatggatgatatagatcttt
acacaaataattacaaaaccgataaatggaaatggataagcgtatgaaatctctcgcaatgaccgctttctttgggga
gctaagcacattagatattatggcattgataatgtctatatttaaacgccatccaaacaataccattttttcagtggataag
gatggtcagtttatgattgatttcgaatacgataattataaggcttctcaatatttggatctgaccctcactccgatatctgga
gatgaatgcaagactcacgcatcgagtatagccgaacaattggcgtgtgtggatattattaaagaggatattagcgaa
tatatcaaaactactccccgtcttaaacgatttataaaaaaataccgcaatagatcagatactcgcatcagtcgagata
cagaaaagcttaaaatagctctagctaaaggcatagattacgaatatataaaagacgcttgttaataagtaaatgaaa
aaaaactagtcgtttataataaaacacgatatggatgccaacgtagtatcatcttctactattgcgacgtatatagacgct
ttagcgaagaatgcttcggaattagaacagaggtctaccgcatacgaaataaataatgaattggaactagtatttatta
agccgccattgattactttgacaaatgtagtgaatatctctacgattcaggaatcgtttattcgatttaccgttactaataag
gaaggtgttaaaattagaactaagattccattatctaaggtacatggtctagatgtaaaaaatgtacagttagtagatgc
tatagataacatagtttgggaaaagaaatcattagtgacggaaaatcgtcttcacaaagaatgcttgttgagactatcg
acagaggaacgtcatatatttttggattacaagaaatatggatcctctatccgactagaattagtcaatcttattcaagca
aaaacaaaaaactttacgatagactttaagctaaaatattttctaggatccggtgcccagtctaaaagttctttattacac
gctattaatcatccaaagtcaaggcctaatacatctctggaaatagaatttacacctagagacaatgaaacagttccat
atgatgaactaataaaggaattgacgactctctcgcgtcatatatttatggcttctccagagaatgtaattctttctccgcct
attaacgcgcctataaaaacctttatgttgcctaaacaagatatagtaggtttggatctggaaaatctatatgccgtaact
aagactgacggcattcctataactatcagagttacatcaaacgggttgtattgttattttacacatcttggttatattattaga
tatcctgttaagagaataatagattccgaagtagtagtctttggtgaggcagttaaggataagaactggaccgtatatct
cattaagctaatagagcctgtgaatgcaatcaatgatagactagaagaaagtaagtatgttgaatctaaactagtggat
atttgtgatcggatagtattcaagtcaaagaaatacgaaggtccgtttactacaactagtgaagtcgtcgatatgttatct
acatatttaccaaagcaaccagaaggtgttattctgttctattcaaagggacctaaatctaacattgattttaaaattaaa
aaggaaaatactatagaccaaactgcaaatgtagtatttaggtacatgtccagtgaaccaattatctttggagagtcgt
ctatctttgtagagtataagaaatttagcaacgataaaggctttcctaaagaatatggttctggtaagattgtgttatataac
ggcgttaattatctaaataatatctattgtttggaatatattaatacacataatgaagtgggtattaagtccgtggttgtacct
attaagtttatagcagaattcttagttaatggagaaatacttaaacctagaattgataaaaccatgaaatatattaactca
gaagattattatggaaatcaacataatatcatagtcgaacatttaagagatcaaagcatcaaaataggagatatcttta
acgaggataaactatcggatgtgggacatcaatacgccaataatgataaatttagattaaatccagaagttagttatttt
acgaataaacgaactagaggaccgttgggaattttatcaaactacgtcaagactcttcttatttctatgtattgttccaaaa
catttttagacgattccaacaaacgaaaggtattggcgattgattttggaaacggtgctgacctggaaaaatacttttatg
gagagattgcgttattggtagcgacggatccggatgctgatgctatagctagaggaaatgaaagatacaacaaatta
aactctggaattaaaaccaagtactacaaatttgactacattcaggaaactattcgatccgatacatttgtctctagtgtc
agagaagtattctattttggaaagtttaatatcatcgactggcagtttgctatccattattcttttcatccgagacattatgcta
ccgtcatgaataacttatccgaactaactgcttctggaggcaaggtattaatcactaccatggacggagacaaattatc
aaaattaacagataaaaagacttttataattcataagaatttacctagtagcgaaaactatatgtctgtagaaaaaata
gctgatgatagaatagtggtatataatccatcaacaatgtctactccaatgactgaatacattatcaaaaagaacgata
tagtcagagtgtttaacgaatacggatttgttcttgtagataacgttgatttcgctacaattatagaacgaagtaaaaagttt
attaatggcgcatctacaatggaagatagaccatctacaagaaactttttcgaactaaatagaggagccattaaatgt
gaaggtttagatgtcgaagacttacttagttactatgttgtttatgtcttttctaagcggtaaataataatatggtatgggttct
gatctcccagttctaaatgcattaaataattccaatagagcgatttttgttcctataggaccttccaactgtggatactctgt
attgttaatagatatattaatacttttgtcgggtaacagaggttctacgtcttttaaaaataaaagtttgataacatctggcct
gttcataaataaaaacttggcgattctatatatactcttattatcaaatctagccattgtcttatagatgtgagctactgtagg
tgtaccatttgattttctttctaatactatatatttctctcgaagaagttcttgcacatcatctgggaataaaatactactgttga
gtaaatcagttattttttttatatcgatattgatggacatttttatagttaaggataataagtatcccaaagtagataacgacg
ataacgaagtatttatacttttaggaaatcacaatgactttatcagattaaaattaacaaaattaaaggagcatgtatttttt
tctgaatatattgtgactccagatacatatggatctttatgcgtcgaattaaatgggtctagttttcagcacggcggtagat
atatagaggtggaggaatttatagatgctggaagacaagttagatggtgttctacatccaatcatatatctaaagatata
cccgaagatatgcacactgataaatttgtcatttatgatatatacacttttgacgctttcaagaataaacgattggtattcgt
acaggtacctccgtcgttaggagatgatagtcatttgactaatccgttattgtctccgtattatcgtaattcagtagccaga
caaatggtcaatgatatgatttttaatcaagattcatttttaaaatatttattagaacatctgattagaagccactatagagttt
ctaaacatataacaatagttagatacaaggataccgaagaattaaatctaacgagaatatgttataatagagataagt
ttaaggcgtttgtattcgcttggtttaacggcgtttcggaaaatgaaaaggtactagatacgtataaaaaggtatctaattt
gatataatgaattcagtgactgtatcacacgcgccatatactattacttatcacgatgattgggaaccagtaatgagtca
attggtagagttttataacgaagtagccagttggctgctacgagacgagacgtcgcctattcctgataagttctttataca
gttgaaacaaccgcttagaaataaacgagtatgtgtgtgcggtatagatccgtatccgaaagatggaactggtgtacc
gttcgaatcaccaaattttacaaaaaaatcaattaaggagatagcttcatctatatctagattaaccggagtaattgatta
taaaggttataaccttaatataatagacggggttataccctggaattattacttaagttgtaaattaggagaaacaaaaa
gtcacgcgatctactgggataagatttccaagttactgctgcagcatataactaaacacgttagtgttctttattgtttgggt
aaaacagatttctcgaatatacgggccaagttagaatccccggtaactaccatagtcggatatcatccagcggctaga
gaccgccaattcgagaaagatagatcatttgaaattatcaacgttttactggaattagacaacaaggcacctataaatt
gggctcaagggtttatttattaatgctttagtgaaattttaacttgtgttctaaatggatgcggctattagaggtaatgatgtta
tctttgttcttaagactataggtgtcccgtcagcgtgcagacaaaatgaagatccaagatttgtagaagcatttaaatgcg
acgagttagaaagatatattgagaataatccagaatgtacactattcgaaagtcttagggatgaggaagcatactctat
agtcagaattttcatggatgtagatttagacgcgtgtctagacgaaatagattatttaacggctattcaagattttattatcg
aggtgtcaaactgtgtagctagattcgcgtttacagaatgcggcgccattcatgaaaatgtaataaaatccatgagatct
aatttttcattgactaagtctacaaatagagataaaacaagttttcatattatctttttagacacgtataccactatggatac
attgatagctatgaaacgaacactattagaattaagtagatcatctgaaaatccactaacaagatcgatagacactgc
cgtatataggagaaaaacaactcttcgggttgtaggtactaggaaaaatccaaattgcgacactattcatgtaatgca
accaccgcatgataatatagaagattacctattcacttacgtggatatgaacaacaatagttattacttttctctacaaca
acgattggaggatttagttcctgataagttatgggaaccagggtttatttcattcgaagacgctataaaaagagtttcaaa
aatattcattaattctataataaactttaatgatctcgatgaaaataattttacaacggtaccactggtcatagattacgtaa
caccttgtgcattatgtaaaaaacgatcgcataaacatccgcatcaactatcgttggaaaatggtgctattagaatttac
aaaactggtaatccacatagttgtaaagttaaaattgttccgttggatggtaataaactgtttaatattgcacaaagaatttt
agacactaactctgttttattaaccgaacgaggagaccatatagtttggattaataattcatggaaatttaacagcgaag
aacccttgataacaaaactaattttgtcaataagacatcaactacctaaggaatattcaagcgaattactctgtccaag
aaaacgaaagactgtagaagctaacatacgagacatgttagtagattcagtagagaccgatacctatccggataaa
cttccgtttaaaaatggtgtattggacctggtagacggaatgttttactctggagatgatgctaaaaaatatacgtgtactg
tatcaaccggatttaaatttgacgatacaaagttcgtcgaagacagtccagaaatggaagagttaatgaatatcattaa
cgatatccaaccattaacggatgaaaataagaaaaatagagagctatatgaaaaaacattatctagttgtttatgcggt
gctaccaaaggatgtttaacattcttttttggagaaactgcaactggaaagtcgacaaccaaacgtttgttaaagtctgct
atcggtgacctgtttgttgagacgggtcaaacaattttaacagatgtattggataaaggacctaatccatttatcgctaac
atgcatttgaaaagatctgtattctgtagcgaactacctgattttgcctgtagtggatcaaagaaaattagatctgacaat
attaaaaagttgacagaaccttgtgtcattggaagaccgtgtttctccaataaaattaataatagaaaccatgcgacaa
tcattatcgatactaattacaaacctgtttttgataggatagataacgcattaatgagaagaattgccgtcgtgcgattca
gaacacacttttctcaaccttctggtagagaggctgctgaaaataatgacgcgtacgataaagtcaaactattagacg
aggggttagatggtaaaatacaaaataatagatatagattcgcatttctatacttgttggtgaaatggtacagaaaatat
catgttcctattatgaaactatatcctacacccgaagagattcctgactttgcattctatctcaaaataggtactctgttagta
tctagctctgtaaagcatattccattaatgacggacctctccaaaaagggatatatattgtacgataatgtggtcactcttc
cgttgactactttccaacagaaaatatccaagtattttaattctagactatttggacacgatatagagagcttcatcaatag
acataagaaatttgccaatgttagtgatgaatatctgcaatatatattcatagaggatatttcatctccgtaaatatatgctc
atatatttatagaagatatcacatatctaaatgaataccggaatcatagatttatttgataatcatgttgatagtataccaac
tatattacctcatcagttagctactctagattatctagttagaactatcatagatgagaacagaagcgtgttattgttccatat
tatgggatcaggtaaaacaataatcgctttgttgttcgccttggtagcttccagatttaaaaaggtttacattctagtgccta
atatcaacattttgaaaatttttaattataatatgggtgtagctatgaacttgtttaatgacgaattcatagctgagaatatctt
tattcattccacaacaagtttttattctcttaattataacgataacgtcattaattataacggattatctcgctacaataactct
atttttatcgttgatgaggcacataatatctttgggaataatactggagaacttatgaccgtgataaaaaataaaaacaa
gattccttttctactattgtctggatctcccattactaacacacctaatactctgggtcatattatagatttaatgtccgaagag
acgatagattttggtgagattattagtcgtggtaagaaagtaattcagacacttcttaacgaacgcggtgtgaatgtactt
aaggatttgcttaaaggaagaatatcatattacgaaatgcctgataaagatctaccaacgataagatatcacggacgt
aagtttctagatactagagtagtatattgtcacatgtctaaacttcaagagagagattatatgattactagacgacagcta
tgttatcatgaaatgtttgataaaaatatgtataacgtgtcaatggcagtattgggacaacttaatctgatgaataatttag
atactttatttcaggaacaggataaggaattgtacccaaatctgaaaataaataatggcgtgttatacggagaagaatt
ggtaacgttaaacattagttccaaatttaaatactttattaatcggatacagacactcaacggaaaacattttatatactttt
ctaattctacatatggcggattggtaattaaatatatcatgctcagtaatggatattctgaatataatggttctcagggaact
aatccacatatgataaacggcaaaccaaaaacatttgctatcgttactagtaaaatgaaatcgtctttagaggatctatt
agatgtgtataattctcctgaaaacgatgatggtagtcaattgatgtttttgttttcgtcaaacattatgtccgaatcctatact
ctgaaagaggtaaggcatatttggtttatgactatcccagatactttttctcaatacaaccaaattcttggacgatctattag
aaaattctcttacgccgatatttctgaaccagttaatgtatatcttttagccgccgtatattccgatttcaatgacgaagtaa
cgtcattaaacgattacacacaggatgaattgattaatgttttaccatttgacatcaaaaagctgttatatctaaaatttaa
gactaaagaaacgaatagaatatactctattcttcaagagatgtctgaaacgtattctcttccaccacatccatcaattgt
aaaagttttattgggagaattggtcagacaatttttttataataattctcgtattaagtataacgactccaagttacttaaaat
ggttacatcagttataaaaaataaagaagacgctaggaattacatagatgatattgtaaacggtcacttctttgtatcga
ataaagtatttgataaatctcttttatacaaatacgaaaacgatattattacagtaccgtttagactttcctacgaaccatttg
tttggggagttaactttcgtaaagaatataacgtggtatcttctccataaaactgatgaaatatataaagaaataaatgtc
gagctttgttaccaatggataccttccagttacattggaaccacacgagctgacgttagacataaaaactaatattagg
aatgccgtatataagacgtatctccatagagaaattagtggtaaaatggccaagaaaatagaaattcgtgaagacgt
ggaattacctctcggcgaaatagttaataattctgtagttataaacgttccgtgtgtaataacctacgcgtattatcacgttg
gggatatagtcagaggaacattaaacatcgaagatgaatcaaatgtaactattcaatgtggagatttaatctgtaaact
aagtagagattcgggtactgtatcatttagcgattcaaagtactgcttttttcgaaatggtaatgcgtatgacaatggcag
cgaagtcactgccgttctaatggaggctcaacaaggtatcgaatctagttttgtttttctcgcgaatatcgtcgactcataa
aaaagagaatagcggtaagtataaacacgaatactatggcaataattgcgaatgttttattctcttcgatatatttttgata
atatgaaaaacatgtctctctcaaatcggacaaccatctcataaaatagttctcgcgcgctggagaggtagttgctgctc
gtataatctccccagaataatatacttgcgtgtcgtcgttcaatttatacggatttctatagttctctgttatataatgcggttttc
catcatgattagacgacgacaatagtgttctgaatttagatagttgatcagaatgaatgtttattggcgttggaaaaattat
ccatacagcgtctgcagagtggttgatagttgttcctagatatgtaaaataatccaacttactaggcagcaaattgtctag
ataaaatactgaatcaaacggtgcagacgtattggcggatctaatggaatccaattgattaactatcttttgaaaatata
catttttatgatccaatacttgtaagaatatagaaataatgataagtccatcatcgtgtttttttgcctcttcataagaactata
ttttttcttattccaatgaacaagattaatctctccagagtatttgtacacatctatcaagtgattggatccataatcgtcttcct
ttccccaatatatatgtagtgatgataacacatattcattggggagaaaccctccacttatatatcctcctttaaaattaatc
cttactagttttccagtgttctggatagtggttggtttcgactcattataatgtatgtctaacggcttcaatcgcgcgttagaaa
ttgcttttttagtttctatattaataggagatagttgttgcggcatagtaaaaatgaaatgataactgtttaaaaatagctctta
gtatgggaattacaatggatgaggaagtgatatttgaaactcctagagaattaatatctattaaacgaataaaagatatt
ccaagatcaaaagacacgcacgtgtttgctgcgtgtataacaagtgacggatatccgttaataggagctagaagaac
ttcattcgcgttccaggcgatattatctcaacaaaattcagattctatctttagagtatccactaaactattacggtttatgta
ctacaatgaactaagagaaatctttagacggttgagaaaaggttctatcaacaatatcgatcctcactttgaagagtta
atattattgggtggtaaactagataaaaaggaatctattaaagattgtttaagaagagaattaaaagaggaaagtgat
gaacgtataacagtaaaagaattcggaaatgtaattctaaaacttacaacgcgcgataaattatttaataaagtatatat
aggttattgcatggcgtgttttattaatcaatcgttggaggatttatcgcatactagtatttacaatgtagaaattagaaaga
ttaaatcattaaatgattgtattaacgacgataaatacgaatatctgtcttatatttataatatgctagttaatagtaaatgaa
cttttacagatctagtataattagtcagattattaagtataatagacgactagctaagtctattatttgcgaggatgactctc
aaattattacactcacggcattcgttaaccaatgcctatggtgtcataaacgagtatccgtgtccgctattttattaactact
gataacaaaatattagtatgtaacagacgagatagttttctctattctgaaataattagaactagaaacatgtttagaaa
gaaacgattatttctgaattattccaattatttgaacaaacaggaaagaagtatactatcgtcatttttttctctagatccagc
tactgctgataatgatagaatagacgctatttatccgggtggcatacccaaaaggggtgagaatgttccagagtgtttat
ccagggaaattaaagaagaagttaatatagacaattcttttgtattcatagacactcggttttttattcatggcatcataga
agataccattattaataaattttttgaggtaatcttctttgtcggaagaatatctctaacgagtgatcaaatcattgatacattt
aaaagtaatcatgaaatcaaggatctaatatttttagatccgaattcaggtaatggactccaatacgaaattgcaaaat
atgctctagatactgcaaaactcaaatgttatggccatagaggatgttattacgaatcattaaaaaaattaactgaggat
gattgattagaaaatataaattaatttaccatcgtgtatttttataacgggattgtccggcatatcatgtagatagttaccgtc
tacatcgtatactcgaccatctacgcctttaaatcctctatttattgacattaatctattagaattggaataccaaatattagt
accctcaattagtttattggtaatatttttgttagacgatagatcgatggctcttgaaaccaaggttttccaaccggactcatt
gtcgatcggtgagaagtctttttcattagcatgaatccattctaatgatgtatgtttaaacactctaaacaattggacaaatt
cttttgatttgctttgaatgatttcaaataggtcttcgtctacagtaggcataccattagataatctagccattataaagtgca
cgtttacatatctacgttctggaggagtaagaacgtgactattgagacgaatggctcttcctactatctgacgaagagac
gcctcgttccatgtcatatctaaaatgaagatatcattgattgagaagaagctaataccctcgcctccactagaagaga
atacgcatgttttaatgcattctccgttagtgtttgattcttggttaaactcagccaccgccttgattctagtatcttttgttctag
atgagaactctatattagagataccaaagactttgaaatatagtaataagatttctattcctgactgattaacaaatggttc
aaagactagacatttaccatgggatgctaatattcccaaacatacatctataaatttgacgcttttctcttttaattcagtaa
atagagagatatcagccgcactagcatcccctcccaatagttctccccttttaaaggtatctaatgcagatttagaaaatt
ctctatctcttaatgaatttttaaaatcattatatagtgttgctatctcttgcgcgtattcgcccggatcacgattttgtctttcag
gaaagctatcgaacgtaaacgtagtagccatacgtctcagaattctaaatgatgatatacctgtttttatttcagcgagttt
agccttttgataaatttcttcttgctttttcgacatattaacgtatcgcattaatactgttttcttagcgaatgatgcagacccttc
tacgtcatcaaaaatagaaaactcgttattaactatgtacgaacataggcctcctagtttggagactaattctttttcatca
actagacgtttattctcaaatagcgattggtgttgtaaggatcctggtcgtagtaagttaaccaacatggtgaattcttgca
cactattgacgataggtgtagccgataaacaaatcatcttatggttttttaatgcgatggtcttagataaaaaattatatact
gaacgagtaggacggatcttaccatcttctttgattaatgatttagaaatgaagttatgacattcatcaataatgacgcat
attctactcttggaattaatagttttgatattagtaaaaaatttatttctaaaattttgatcatcgtaattaataaaaatacaatc
cttcgttatctctggagcgtatctgagtatagtgttcatccaaggatcttctatcaaagcctttttcaccaataagataatag
cccaattcgtataaatatccttaagatgtttgagaatatatacagtagtcattgttttaccgacacccgtttcatggaacaat
aaaagagaatgcatactgtctaatcctaagaaaactcttgctacaaaatgttgataatccttgaggcgtactacgtccg
accccatcatttcaacaggcatattagtagttctgcgcaatgcataatcgatataggccgcgtgtgatttactcatttatga
gtgataagtaataactatgttttaaaaatcacagcagtagtttaactagtcttctctgatgtttgttttcgatactttttgaatca
gaagtcatactagaataaagcaacgagtgaacgtaatagagagcttcgtatactctattcgaaaactctaagaactta
ttaatgaattccgtatccactggattgtttaaaatactaaattgaacactgttcacatccttccaagaagaagacttagtga
cggacttaacatgagacataaataaatccaaattttttttacaaacatcactagccaccataatggcgctatcttttaacc
agctatcgcttacgcattttagcagtctaacatttttaaagagactacaatatattctcatagtatcgattacacctctaccg
aataaagttggaagtttaataatacaatatttttcgtttacaaaatcaaataatggtcgaaacacgtcgaaggttaacatc
ttataatcgctaatgtatagattgttttcagtgagatgattattagatttaatagcatctcgttcacgtttgaacagtttattgcgt
gcgctgaggtcggcaactacggcgtccgctttagtactcctcccataatactttacgctattaatctttaaaatttcataga
ctttatctagatcgctttctggtaacatgatatcatgtgtaaaaagttttaacatgtcggtcggcattctatttagatcattaact
ctagaaatctgaagaaagtaattagctccgtattccagactaggtaatgggcttttacctagagacagattaagttctgg
caatgtttcataaaatggaagaaggacatgcgttccctcccggatattttttacaatttcatccatttacaactctatagtttg
ttttcattattattagttattatctcccataatcttggtaatacttaccccttgatcgtaagataccttatacaggtcattacatac
aactaccaattgtttttgtacataatagattggatggttgacatccatggtggaataaactactcgaacagatagtttatctt
tccccctagatacattagccgtaatagttgtcggcctaaagaatatctttggtgtaaagttaaaagttagggttcttgttcca
ttattgctttttgtcagtagttcattataaattctcgagatgggtccgttctctgaatatagaacatcatttccaaatctaacttct
agtctagaaataatatcggtcttattcttaaaatctattcccttgatgaagggatcgttaatgaacaaatccttggcctttgat
tcggctgatctattatctccgttatagacgttacgttgactagtccaaagacttacaggaatagatgtatcgatgatgttgat
actatgtgatatgtgagcaaagattgttctcttagtggcatcactatatgttccagtaatggcggaaaactttttagaaatgt
tatatataaaagaattttttcgtgttccaaacattagcagattagtatgaagataaacactcatattatcaggaacattatc
aatttttacatacacatcagcatcttgaatagaaacgataccatcttctggaacctctacgatctcggcagactccggat
aaccagtcggtggaccatcgctaacaataactagatcatccaacaatctactcacatatgcatctatataatctttttcat
cttgtgagtaccctggatacgaaataaatttattatccgtatttccataataaggtttagtataaacagagagcgatgttgc
cgcatgaacttcagttacagtcgccgttggttggtttatttgacctattactctcctaggtttctctataaatgatggtttaatttg
tacattcttaaccatatatccaataaagctcaattcaggaacataaacaaattctttgttgaacgtttcaaagtcgaacga
agagtcacgaataacgatatcggatactggattgaaggttaccgttacggtaatttttgaatcggatagtttaagactgct
gaatgtatcttccacatcaaacggagttttaatataaacgtatactgtagatggttctttaatagtgtcattaggagttaggc
caatagaaatatcattaagttcactagaatatccagagtgtttcaaagcaattgtattattgatacaattattatataattctt
cgccctcaatttcccaaataacaccgttacacgaagagatagatacgtgattaatacatttatatccaacatatggtacg
taaccgaatcttcccatacctttaacttctggaagttccaaactcagaaccaaatgattaagcgcagtaatatactgatc
cctaatttcgaagctagcgatagcctgattgtctggaccatcgtttgtcataactccggatagagaaatatattgcggcat
atataaagttggaatttgactatcgactgcgaagacattagaccgtttaatagagtcatccccaccgatcaaagaatta
atgatagtattattcattttctatttaaaatggaaaaagcttacaataaactccgtagagaaatatctataatttgtgagttttc
cttaaagtaacagcttccgtaaacgccgtctttatctcttagtaagtttattgtatttataaccttttccttatcttcatagaatac
taaaggcaacaaagaaatttttggttcttctctaagagctacgtgagacttaaccatagacgccaacgaatccctacat
attttagaacagaaatacccaacttcaccacccttgaatgtctcaatactaataggtttaaaaaccaaatcttgattaca
aaaccaacacttatcaattacactatttgtcttaatagacacatctgccatagatttataatactttggtagtatacaagcg
agtgcttcttctttagcgggcttaaagactgctttaggtgctgaaataaccacatctggaaggcttactcgcttagccattt
aattacggaactatttttttatacttctaatgagcaagtagaaaacctctcatctacaaaaacatactcgtgtccataatcc
tctaccatagttacacgttttttagatctcatatgtgctaaaaagttttcccatactaattggttactattatttttcgtataattttta
acagtttgaggttttagatttttagttacagaagtgatatcgaatattttatccaaaaagaatgaataattaattgtcttagaa
ggagtgttttcttggcaaaagaataccaagtgcttaaatatttctactacttcattaatcttttctgtactcagattcagtttctc
atcttttacttgattgattatttcaaagactaacttataatcctttttatttattctctcgttagccttaagaaaactagatacaaa
atttgcatctacatcatccgtggatatttgatttttttccatgatatccaagagttccgagataatttctccagaacattgatga
gacaataatctccgcaatacatttctcaaatgaataagtttattagacacatggaagtttgactttttttgtacctttgtacattt
ttgaaataccgactcgcaaaaaatacaatattcatatccttgttcagatactataccgttgtgtctacaaccgctacataat
cgtagattcatgttaacactctacgtatctcgtcgtccaatattttatataaaaacattttatttctagacgttgccagaaaat
cctgtaatatttttagttttttgggctgtgaataaagtatcgccctaatattgttaccgtcttccgccaatatagtagttaaattat
ccgcacatgcaaaagaacaccgcttaggcggattcagtacaatgttatatttttcgtaccaactcatttaaatatcataat
ctaaaatagttctgtaatatgtctagcgctaatatattgatcataatcctgtgcataaattaagatacaacaatgtctcgaa
atcatcgacatggcttcttccatagttagaagatcgtcgtcaaagttagcaacgtgattcatcaacatttgctgttttgagg
cagcaaatactgaaccgtcgccattcaaccattcataaaaaccatcgtctgaatccattgataatttcttgtactggtttttg
agagctcgcatcaatctagcatttctagctcccggattgaaaacagaaagaggatcgtacatccagggtccattttctgt
aaatagaatcgtataatgtcccttcaagaagatatcagacgatccacaatcaaagaattggtctccgagtttgtaacaa
actgcggactttaacctatacatgataccgtttagcatgatttctggtgatacgtcaatcggagtatcatctattagagatct
aaagccggtgtaacattctccaccaaacatattcttattctgacgtcgttctacataaaacatcattgctccattaacgata
acaggggaatgaacagcactacccatcacattagttcccaatggatcaatgtgtgtaactccagaacatcttccatatc
ctatgttaggaggagcgaacaccactcttccactattgccatcgaatgccatagaataaatatccttggaattgataga
aatcggactgtcggatgttgtgatcatcttcataggattaacaactatgtatggtgccgcctgaagtttcatatcgtaactg
atgccgtttataggtctagccacagaaaccaacgtaggtctaaatccaactatagacaaaatagaagccaatatctgt
tcctcatctgtcataacttgagagcatccagtatgaataatcttcattagatggggatctaccgcatcatcatcgttacaat
aaaaaattcccattctaatgttcataattgcttttctaatcatggtatgcatgtttgctctctgaatctctgtggaaattagatct
gatacacctgtaatcactatcggattatcctccgtaagacgattaaccaacaacatataattataagactttacttttctaa
attcataaagttgctggattaggctataggtgtctccatgtacatacgcgttctcgagcgcaggaagtttaataccgaata
gtgccatcagaataggatgaatatagtaattagtttctggttttctataaataaaagacaaatcttgtgaactagacatatc
ggtaaaatgcatggattggaatcgtgtagtcgacagaagaatatgatgattagatggagagtatattttatctaactcttt
gagttggtcaccgattctaggactagctcgagaatgaataagtactaaaggatgagtacatttcacagaaacactagc
attgttcaatgtgctctttacatgggtaaggagttgaaatagctcgtttctatttgttctgacaatatttagtttattcataatgtta
agcatatcctgaatagtaaagttagatgtgtcatacttgttagtagttagatatttagcaattgcattcccatcatttctcaatc
tcgtactccaatcatgtgtagatgctacttcgtcgatggaaaccatacaatcctttttgataggctgttgagattgattatttc
ctgcacgtttaggtttggtacgttgatttctagcccctgcggatataaagtcatcgtctacaatttgggacaatgaattgcat
acactacaagacaaagatttatcagaagtgtgaatatgatcttcatctaccaaagaaagagtttgattagtataactag
attttagtcctgcgttagatgttaaaaaaacatcgctattgaccacggcttccattatttatattcgtagtttttactcgaaagc
gtgattttaatatccaatcttattacttttggaatcgttcaaaacctttgactagttgtagaatttgatctattgccctacgcgtat
actcccttgcatcatatacgttcgtcaccagatcgtttgtttcggcctgaagttggtgcatatctctttcaacattcgacatga
gatccttaagggccatatcgtctagattttgttgagatgctgctcctggatttggattttgttgtgctgttgtacatactgtacca
ccagtaggtgtaggagtacatacagtggccacaataggaggttgaggaggtgtaaccgttggagtagtacaagaaa
tatttccatccgattgttgtgtacatgtagttgttggtaacgtctgagaaggttgggtagatggcggtgtcgtcgtcttttgatc
tttattaaatttagagataatatcctgaacagcattgctcggcgtcaacgctggaaggagtgaactcgccggcgcatca
gtatcttcagacagccaatcaaaaagattagacatatcagatgatgtattagtttgttgtcgtggttttggtgtaggaacag
tactactaggtagaagaataggagccggtgtagctgttggaaccggctgtggagttatatgaatagttggttgtagcggt
tggataggctgtctgctggcgaccgtcatattatctctagctagttgttctcgcaactgtctttgataatacgactcttgagac
tttagtcctatttcaatcgcttcatcctttttcgtatccggatccttttcttcagaataatagattgacgactttggtgtagaggat
tctgccagcccctgtgagaacttgttaaagaagtccatttaaggctttaaaattgaattgcgattataagattaaatggca
gacacagacgatattatcgactatgaatccgatgatctcaccgaatacgaggatgatgaagaagaggaagaagat
ggagagtcactagaaactagtgatatagatcccaaatcttcttataagattgtagaatcagcatccactcatatagaag
atgcgcattccaatcttaaacatatagggaatcatatatctgctcttaaacgacgctatactagacgtataagtctatttga
aatagcgggtataatagcagaaagctataacttgcttcaacgaggaagattacctctagtttcagaattttctgacgaa
acgatgaagcaaaatatgctacatgtaattatacaagagatagaggagggttcttgtcctatagtcatcgaaaagaac
ggagaattgttgtcggtaaacgattttgacaaagatggtctaaaattccatctagactatattatcaaaatttggaaacttc
aaaaacgatattagaatttatacgaatatcgttctctaaatgtcacaatcaagtctcgcatgttcagcaatttattgtcgtac
tttatatcgtgttcattaacgatatcttgcaaaatagtaatgattctatcttccttcgatagatattcttcagagattattgtcttat
attttttcttgttatccgatatgaatttgataagactttgaacattattgatacccgtctgtttaattttttctacagatattttagtttt
ggcagattctatcgtatctgtcaatagacatccaacatcgacattcgacgtcaattgtctataaatcaacgtataaatttta
gaaataacattagcgaattgttgtgcattgatgtcgttattctgaaacagtatgattttaggtagcattttcttaacaaagag
aacgtatttattgttactcagttgaacagatgatatatccagattactaacgcatctgattccgtataccaaactttcagaa
gaaatggtgtacaattgtttgtattcattcaatgtctctttttcagaaattagtttagagtcgaatactgcaataattttcaaga
gatagttttcatcagataagattttatttagtgtagatatgataaaactattgttttgttggagaacttgatacgccgcgttctct
gtagtcgacgctctcaaatgggaaacaatctccattatttttttggaatcggatactatatcttcggtatcttgacgcagtct
agtatacatagagttaagagagattagagtttgtacattaagcaacatgtctctaaatgtggctacaaacttttcctttttca
cataatctagtttattatataccgatttcacaacggcaccagatttaaggaaccagaatgaaaaactctgataactaca
atatttcatcatagttacgattttatcatcttctatagttggtgtaatagcgcatacctttttctccaagactggaaccaacgtc
ataaaaatgtttaaatcaaaatccatatcaacatctgatgcgctaagaccagtctcgcgttcaagattatctttactaatg
gtgacgaactcatcatatagaactctaagtttgtccattatttatttacagatttagttgtttaatttatttgtgctcttccagagtt
gggatagtatttttctaacgtcggtattatattattaggatctacgttcatatgtatcataatattaatcatccacgttttgataa
atctatctttagcttctgaaataacgtatttaaacaaaggagaaaaatatttagctacggcatcagacgcaataacatttt
ttgtaaatgtaacgtatttagacgacagatcttcgttaaaaagttttccatctatgtagaatccatcggttgttaacaccattc
ccgcgtcagattgaataggagtttgaatagtttgttttggaaatagatccttcaataacttatagttgggtgggaaaaaatc
gattttatcactagactctttcttttttactatcattacctcatgaactatttcttgaatgagtatatgtattttctttcctatatcgga
cgcgttcattggaaaatataccatgtcgttaactataagaatatttttatcctcgtttacaaactgaataatatcagatgtag
ttcgtaaacgaactatatcatcaccagcacaacatctaactatatgatatccactagtttcctttagccgtttattatcttgttc
catattagcagtcattccatcatttaagaaggcgtcaaagataatagggagaaatgacattttggattctgttacgacttt
accaaaattaaggatatacggacttactatctttttctcaacgtcgatttgatgaacacacgatgaaaatgtgcttctatga
gattgatcatgtagaaaacaacaagggatacaatatttccgcatatcatgaaatatattaagaaatcccaccttattata
tttccccaaaggatccatgcatgtaaacattatgccgttatcattaataaagacttctttctcatcggatttgtaaaagttgtt
actgatttttttcattccaggatctagataattaataatgatgggttttctattcttattctttgtattttggcatatcctagaccagt
aaacagtttccactttggtaaaatcagcagacttttgaacgctattaaacatggcattaatggcaataactaaaaatgta
aaatatttttctatgttaggaatatggtttttcactttaatagatatatggtttttggccaaaatgatagatatttttttatccgagg
atagtaaaatattattagtcgccgtctctataaaaatgaagctagtctcgatatccaattttattctagaattgataggagtc
gccaaatgtaccttatacgttatatctcccttgatgcgttccatttgtgtatctatatcggacacaagatctgtaaatagtttta
cgttattaatcatcacggtatcgccgtcgctagataacgctaatgtaccatccaagtcccaaatggagagatttaactgtt
catcgtttagaataaaatgattaccggtcatattaataaagtgttcatcgtatctagataacaacgacttataattaatgtc
caagtcttgaactcgctgaatgatcttttttaacccagttagttttagattggtacgaaatatattgttaaactttgattctaca
gtaatgtccaaatctagttgtggaaatacttccatcaacattgtttcaaacttgataatattattatctacatcttcatacgatc
caaattccggaatagatgtatcacatgctcttgccacccagataaccaaaaagtcacacgctccaggatatacattgt
ataaaaagctatcgttttttagtagtgtttttttctgagtatatacgaagggattaaaaatagtattatcaacgtaactatattc
caaattattcttatgagaatagataataatatcgtccttaatatctaacaaatttcctaaatatccctttaattgagtcattcga
agcgtcaatagaatatgtctcttaactatttccggctgttgtatatttaaatgacttcgtaaaaaataatatatgggcgactt
ctcatctatgtaatcatatggagtgagatatagggctcgttctacctcctgccccttacccacctgtaataccaattgcgg
acttactatatatcgcatatttatatcgtggggtaaagtgaaaatctactaccgatgatgtaagtcttacaatgttcgaacc
agtaccagatcttaatttggaggcctccgtagaactaggggaggtaaatatagatcaaacaacacctatgataaagg
aaaatagcggttttatatcccgtagtagacgtctattcgcccatagatctaaggatgatgagagaaaactagcactacg
attctttttacaaagactttattttttagatcatagagagattcattatttgttcagatgcgttgacgctgtaaaagacgtcact
attaccaaaaaaaataacattatcgtggcgccttatatagcacttttaactatcgcatcaaaaggatgcaaacttacag
aaacaatgattgaagcattttttccagaactatataatgaacatagtaagaaatttaaattcaactctcaagtatccatca
tccaagaaaaactcggataccagtttggaaactatcacgtttatgattttgaaccgtattactctacagtagctctggctat
tcgagatgaacattcatctggcatttttaatatccgtcaagagagttatctggtaagttcattatctgaaataacatatagat
tttatctaattaatctaaaatctgatcttgttcaatggagtgctagtacgggcgctgtaattaatcaaatggtaaatactgtat
tgattacagtgtatgaaaagttacaactggtcatagaaaatgattcacaatttacatgttcattggctgtggaatcaaaac
ttccaataaaattacttaaagatagaaatgaattatttacaaaattcattaacgagttaaaaaagaccagttcattcaag
ataagcaaacgcgataaggatacgctactaaaatattttacttaggactggagttagaatttatagacgactcatttcgtt
tatcattgttactattattactattactatcattattagtgttggcattattagtattcttcttgtcatcttgttcagaaatatacagc
aatgctatacctaatactaaatacattatcatgctcgcaatggctctaacaacaacgaaccaaaatgaatttggtcgta
gcttttgttcacaaaaatacataaagaaatgtctacataaatctatggcgccattggctacttgaaatagcgccagtcct
cctacagattttaatatagctgtataacatgacatttattcatcatcaaaagagacagagtcaccatctgtcatatttagatt
ttttttcatgtgttcaaagtatcctctactcatttcattataatagtttatcatacttagaattttaggacggatcaatgagtaag
acttgactagatcgtcagtagtaatttgtgcatcgtctattctgcatccgcttcgtcgaataatgtatagcatcgctttgagat
tctccatagctatcaagtctttatacaatgacatggaaatatctgtgaatactttatacttctccaacatcgatgccttaacat
catcgcctactttagcattgaaaatacgttctattgtgtagatggatgtagcaagatttttaaacaacaatgccatcttaca
cgatgattgcctcaagtctccaatcttttgtttagaacgattagctacagagttcaacgcttggctgactagcatattattat
ctttagaaattgtattcttcaatgaggcgtttatcatatctgtgatttcgttagtcatattacagtctgactgggttgtaatgttat
ccaacatatcacctatggatacggtacacgtaccagcatttgtaataatcctatctaagatgttgtatggcattgcgcag
aaaatatcttctcctgtaatatctccactctcgataaatctactcagattattcttaaatgccttattctctggagaaaagata
tcagtgtccatcatttcattaatagtatacgcagaaaagataccacgagtatcaattctatccaagatacttatcggttcc
gagtcacagataatggtttcctctccttcgggagatcctgcatagaaatatctaggacaatagtttctatactgtctgtaac
tctgataatctctaaagtcactaactgataccatgaaattgagaagatcaaacgctgaagtaatcaatttttctgcctcgtt
tttactacaactagttttcatcaatgtagcgacgatgtattgtttagttactcttggtctaatactgatgatagagatattattgc
ttcccataatggatcttctagtagtcaccttaaagcccattgatgcgaatagcagatagataaagtcttggtatgactcctt
tctaatatagtacggactacctttgtcacccaactttatacccacataagccataacaacctctttaatagccgtttcatga
ggtttatcagccatgagcctgagtagttggaagaatctcatgaatcccgtctcagaaagtcctatatgcatgatagattta
tctttcctgggaaactctcgtatagtcatagatgaaatactcttcaaagtttctgaaataagattagtaacagtcttacctcc
gactactctgggtaacaaacatactctaataggtgttttctctgcggagataatatcagaaaggatagagcaataagta
gtattattgtgattataaagaccgaatacataacaggtagaatttataaacatcatgtcctgaaggtttttagacttgtattc
ctcgtaatccataccgtcccaaaacatggatttggtaactttgatagccgtagatctttgttccttcgccaacaggttaaag
aaattaataaagaatttgttgtttctatttatgtccacaaattgcacgtttggaagcgccacggttacattcactgcagcattt
tgaggatcgcgagtatgaagtacgatgttattgtttactggtatatctggaaagaaatctaccagtctaggaataagaga
ttgatatcgcatagaaatagtaaagtttataatctcatcatcgaagagcattttgttaccattgtaataaatatccactctgtc
atatgtataaatgaagtactgttcaaacatgatgagatgtttatatgttggcatagtagtgagatcgacgtttggtaatggc
aatgtattaagattaactccataatgtctagcagcatctgcgatgttataagcgtcgtcaaagcggggtcgatcttgtattg
ttatatattgtctaacacctataagattatcaaaatcttgtctgcttaatacaccgttaacaatttttgccttgaattcttttattgg
tgcattaataacatccttatagaggatgttaaacaaataagtgttatcaaagttaagatctggatatttcttttctgctagaa
catccattgagtcggagccatctggtttaatataaccaccgataaatctagctctgtattctgtatccgtcaatctaatatta
agaaggtgttgagtgaaaggtggaagatcgtaaaagctgtgagtattaatgataggattagtttccgaactaatgttaat
tggggtattaataatatctatatttccagcgttaagtgtaacattaaacagttttaattcacgtgacgtggtatcaattaaata
attaatgcccaatttggatatagcagcctgaagctcatcttgtttagttacggatcctaatgagttattaagcaatatatcga
acggatgaacgaaggttgttttaagttggtcacatactttgtaatctagacatagatgcggaagaacggtagaaactat
acgaaataaatattcagagtcctctaattgatcaagagtaactattgacttaataggcatcatttatttagtattaaatgac
gaccgtaccagtgacggatatacaaaacgatttaattacagagttttcagaagataattatccatctaacaaaaattat
gaaataactcttcgtcaaatgtctattctaactcacgttaacaacgtggtagatagagaacataatgccgccgtagtgtc
atctccagaggaaatatcctcacaacttaatgaagatctatttccagatgatgattcaccggccactattatcgaacgag
tacaacctcatactactattattgacgatactccacctcctacttttcgtagagagttattgatatcggaacaacgtcaaca
acgagaaaaaagatttaatattacagtatcgaaaaatgctgaagcaataatggaatctagatctatgataacttctatg
ccaacacaaacaccatccttgggagtagtttatgataaagataaaagaattcagatgttggaggatgaagtggttaat
cttagaaatcaacgatctaatacaaaatcatctgataatttagataattttaccaaaatactatttggtaagactccgtata
aatcaacagaagttaataagcgtatagccatcgttaattatgcaaatttgaacgggtctcccttatcagtcgaggacttg
gatgtttgttcagaggatgaaatagatagaatctataaaacgattaaacaatatcacgaaagtagaaaacaaaaaat
tatcgtcactaacgtgattattattgtcataaacattatcgagcaagcattgctaaaactcggatttgaagaaatcaaag
gactgagtaccgatatcacttcagaaattatcgatgtggagatcggagatgactgcgatgctgtagcatcaaaactag
gaatcggtaacagtccggttcttaatattgtattgtttatactcaagatattcgttaaacgaattaaaattatttaatttaatac
attcccatatccagacaacaatcgtctggattaatctgttcctgtcgtctcataccggacgacatattaatctttttattagta
ggcatctttttagatggtttctttttcccagcattaactgagtcgatacctagaagatcgtgattgatctctccgaccattcca
cgaacttctaattggccgtctctaacggtaccataaactattttaccagcattagtaacagcttggacaatctgaccatcc
atcgcattgtacgatgtagtagtaactgttgttctacgtctaggagcaccagaagtatttttggagcccttggaggctgatg
tagaagaagacgaggattttgattttggtttacatgtaatacattttgaactctttgattttgtatcacatgcgccggcagtca
catctgtttgagaattaagattattgttgcctcctttgacggctgcatctccaccgatttgcgctagtagatttttaagctgtgg
tgtaatcttattaactgtttcgatataatcatcgtaactgcttctaacggctaaattttttttatccgccatttagaagctaaaaa
tatttttatttatacagaagatttaactagattatacaatgaactaatatgatccttttccagattatttacaaacttggtatttttt
ggttctggaggaggcgaatttaaattcggacttggattcggattttgtgagttcttgatcttattatacatcgagtataggatg
gcgacggtaactgctacacaaataccgatcaacaaaagaataccaatcatttattgacaataacttcactattgatca
agtatgcaatatatcatcttttcactaaataagtagtaataatgattcaacaatgtcgagatatatggacgataataattta
gttcatggaaatatcgctatgattggtatgaatgactccgctaactctgtggggcgcgcagtgctttccccacatagaat
aaattagcattccgactgtgataataataccaagtataaacgccataatactcaatactttccatgtacgagtgggactg
gtagacttactaaagtcaataaaggcgaagatacacgaaagaatcaaaagaatgattccagcgattagcacgccg
gaaaaataatttccaatcataagcatcatgtccatttaactaataaaaattttaaatcgccgaatgaacaaagtggaat
ataaaccatataaaaacaatagtttgtactgcaaaaataatatctatttttgttttcgaagatatggtaaaattaaatagta
gtacacagcatgttataactaacagcagcaacggctcgtaattacttatcatttactagacgaaaaggtggtgggatatt
ttcttgctcaaataatacgaatatatcacccatccattttatgcgatgtttatatactctaatctttaatagatctatagacgac
gggtttaccaacaatatagattttatcgattcatctaatttaaacccttccttaaacgtgaatgatctattatctggcataacg
atgaccctacctgatgaatcggacaatgtactgggccatgtagaataaattatcaacgaattatcgtctacgaacattta
tatcatttgttttaattttaggacgcgaataaatagatataaaatagaaaataacagatattacaaccagtgttatggccg
cgcccaaccaggtaggcagttttattttatcttttactacaggttctcctggatgtacgtcaccaacggcggacgtagttct
agtacaattagacgtaagttccgcttgggaattttttaacgctaaagagttaacgttaatcgtgcacccaacgtatttacat
ctagttctttgaacatcttgattataatataaccattttctatctctagattcgtcggtgcactcatgtaaccaacataccctag
gtcctaaatatttatctccggaattagattttggataattcgcgcaccaacaatttctatttcctttatgatcgttacaaaaga
cgtataatgccgtatccccaaaagtaaaataatcaggacgaataattctaataaactcagaacaatatctcgcatcca
tatgtttggagcaaatatcggaataagtagacatagccggtttccgttttgcacgtaaccattctaaacaattggggtttcc
aggatcgtttctacaaaatccagtcatgaaatcgtcacaatgttctgtcttgtaattattattaaatatttttggacagtgtttg
gtatttgtcttagaacaacattttgccacgctatcactatcgcccaggagataatccttttttataaaatgacatcgttgccc
ggatgctatataatcagtagcgtgttttaaatccttaatatattcaggagttacctcgttctgataatagattaatgatccag
gacgaaatttgaaagaactacatggttctccatgaattaatacatattgtttagcaaattcaggaactataaaactacta
caatgatctatcgacataccatctatcaaacaaaacttgggtttaatttctcccggagatgtttcataatagtacgtataac
tttcttctgcaaacttaacagctctattatattcaggataattaaaacctaattccatatatttgtctcgtatatctgctattcctg
gtgctattttgattctattaagagtaacagctgcccccattcttaataatcgtcagtatttaaactgttaaatgttggtatatca
acatctaccttatttcccgcagtataaggtttgttgcaggtatactgttcaggaatggttacatttatacttcttctatagtcctg
tctttcgatgttcatcacatatgcaaagaacagaataaacaaaataatgtaagaaataatattaaatatctgtgaattcgt
aaatacattgattgccataataattacagcagctacaatacacacaatagacattcccacagtgttgccattacctcca
cgatacatttgagttactaagcaataggtaataactaagctagtaagaggcaatagaaaagatgagataaatatcatc
aatatagagattagaggagggctatatagagccaagacgaacaaaatcaaaccgagtaacgttctaacatcattatt
tttgaagattcccaaataatcattcattcctccataatcgttttgcatcatacctccatctttaggcataaacgattgctgctgt
tcctctgtaaataaatctttatcaagcactccagcacccgcagagaagtcgtcaagcatattgtaatatcttaaataactc
atttatatattaaaaaatgtcactattaaagatggagtataatctttatgccgaactaaaaaaaatgacttgtggtcaacc
cctaagtctttttaacgaagacggggatttcgtagaagttgaaccgggatcatcctttaagtttctgatacctaagggatttt
acgcctctccttccgtaaagacgagtctagtatttgaaacattaacaacgaccgataataaaatcactagtatcaatcc
aacaaatgcgccaaagttatatcctcttcaacgcaaagtcgtatctgaagtagtttctaatatgaggaaaatgatcgaat
caaaacgtcctctatacattactcttcacttggcgtgtggatttggtaagactattaccacgtgttatcttatggctacacac
ggtagaaaaaccgtcatttgcgtacccaataaaatgttaatacatcaatggaagacacaggtagaggcagtcggatt
ggaacataagatatccatagatggagtaagtagtctattaaaggaactaaagactcaaagtccggatgtattaatagt
agtcagtagacatctgacaaacgatgccttttgtaaatatatcaataagcattatgatttgttcatcttggatgaatcacat
acgtataatctgatgaacaatacagcagttacaagatttttagcgtattatcctccgatgatgtgttattttttaactgctaca
cctagaccagctaaccgaatttattgtaacagtattattaatattgccaagttatccgatctaaaaaaaactatctatgcg
gtagatagtttttttgagccatattccacagacaatattagacatatggtaaaacgactagatggaccatctaataaatat
catatatataccgagaagttattatctgtagacgagcctagaaatcaacttattcttaataccctggtagaagaattcaag
tcaggaactattaatcgcattttagttattactaaactacgtgaacatatggtattattctacaaacgattattagatcttttcg
gaccagaggttgtatttataggagacgcccaaaatagacgtactccagatatggtcaaatcaatcaaggaactaaat
agatttatattcgtatccaccttattttattccggtactggtttagatattcctagtttggattcgttgttcatttgctcggcagtaat
caacaatatgcaaatagagcaattactagggagggtatgtcgagaaacagaactattagataggacggtatatgtatt
tcctaacacatccatcaaagaaataaagtacatgataggaaatttcatgcaacgaattattagtctgtctgtagataaac
taggatttaaacaagaaagttatcggaaacatcaagaatccgatcccacttctgtatgtacaacatcctccagagaag
aacgtgtattaaatagaatatttaactcgcaaaatcgttaagaagtttaagcgacgatccgcatgctgcgcaggccagt
gtattacccctcatagtattaatataatccaatgatacttttgtgatgtcggaaatcttaaccaatttagactgacaggcag
aacacgtcatgcaatcatcatcgtcatcgataactgtagtcttgggcttctttttgcggctcttcattccggaacgcacattg
gtgctatccatttaggtagtaaaaaataagtcagaatatgccctataacacgatcgtgcaaaacctggtatatcgtctct
atctttatcacaatatagtgtatcgacatctttattattattgacctcgtttatcttggaacatggaatgggaacatttttgttatc
aacggccacctttgccttaattccagatgttgtaaaattataactaaacagtctatcatcgacacaaatgaaattcttgttt
agacgtttgtagtttacgtatgcggctcgttcgcgtctcattttttcagatattgcaggtactataatattaaaaataagaatg
aaataacataggattaaaaataaagttatcatgacttctagcgctgatttaactaacttaaaagaattacttagtctgtac
aaaagtttgaaattttcagattctgcggctatagaaaagtataattctttggtagaatggggaacatctacttactggaaa
ataggcgtgcaaaaggtagctaatgtcgagacgtcaatatctgattattatgatgaggtaaaaaataaaccgtttaatat
tgatccgggctattacattttcttaccggtatattttgggagcgtctttatttattcgaagggtaaaaatatggtagaacttgg
atctggaaactcttttcaaataccagatgatatgcgaagtgcgtgtaacaaagtattagacagcgataacggaataga
ctttctgagatttgttttgttaaacaatagatggataatggaagatgctatatcaaaatatcagtctccagttaatatatttaa
actagctagtgagtacggattaaacatacccaaatatttagaaattgaaatagaggaagacacattatttgacgacga
gttatactctattatagaacgctcttttgatgataaatttccaaaaatatccatatcgtatattaagttgggagaacttaggc
ggcaagttgtagactttttcaaattctcgttcatgtatattgagtccatcaaggtagatcgtataggagataatatttttattcc
tagcgttataacaaaatcaggaaaaaagatattagtaaaagatgtagaccatttaatacgatctaaggttagagaac
atacatttgtaaaagtaaaaaagaaaaacacattttccattttatacgactatgatgggaacggaacagaaactagag
gagaagtaataaaacgaattatagacactataggacgagactattatgttaacggaaagtatttctctaaggttggtag
tgcaggcttaaagcaattgactaataaattagatattaatgagtgcgcaactgtcgatgagttagttgatgagattaata
aatccggaactgtaaaacgaaaaataaaaaaccaatcagcatttgatttaagcagagaatgtttgggatatccagaa
gcagattttataacgttagttaataacatgcggttcaaaatagaaaattgtaaggttgtaaatttcaatattgaaaatacta
attgtttaaataacccgagtattgaaactatatatggaaactttaaccagttcgtctcaatctttaatatcgtcaccgatgtc
aaaaaaagattattcgagtgaaataatatgcgcctttgatataggtgcaaaaaatcctgccagaactgttttagaagtc
aaggataactccgttagggtattggatatatcaaaattagactggagttctgattgggaaaggcgcatagctaaagattt
gtcacaatatgaatacactacagttcttctagaacgtcagcctagaaggtcgccgtatgttaaatttatctattttattaaag
gctttttatatcatacatcggctgccaaagttatttgcgtctcgcctgtcatgtctggtaattcatatagagatcgaaaaaag
agatcggtcgaagcatttcttgattggatggacacattcggattgcgagactccgttccggatagacgcaaattagacg
atgtagcggatagtttcaatttggctatgagatacgtattagataaatggaatactaattatacaccttataataggtgtaa
atctagaaattacataaaaaaaatgtaataacgttagtaacgccattatggataatctatttacctttctacatgaaatag
aagatagatatgccagaactatttttaactttcatctaataagttgcgatgaaataggagatatatatggtcttatgaaag
aacgcatttcctcagaggatatgtttgataatatagtatataataaagatatacatcctgccattaagaaactagtgtattg
cgacatccaacttactaaacacattattaatcagaatacgtatccggtatttaacgattcttcacaagtgaaatgttgtcat
tatttcgacataaactcagataatagcaatattagctctcgtacagtagagatatttgagagggaaaagtcatctcttgta
tcatatattaaaactaccaataagaagagaaaggtcaattacggcgaaataaagaaaactgttcatggaggcacta
atgcaaattacttttccggtaaaaagtctgacgagtatctgagtactacagttagatccaacattaatcaaccttggatca
aaaccatttctaagagaatgagagtagatatcattaatcactctatagtaacgcgtggaaaaagctctatattacaaac
tatagaaattatttttactaatagaacatgtgtgaaaatattcaaggattctactatgcacattattctatccaaggacaag
gatgaaaaggggtgtatacacatgattgacaaattattctatgtctattataatttatttctgttgttcgaagatatcatccaa
aacgagtactttaaagaagtagctaatgttgtaaaccacgtacttacggctacggcattagatgagaaattattcctaat
taagaaaatggctgaacacgatgtttatggagttagcaatttcaaaatagggatgtttaacctgacatttattaagtcgttg
gatcataccgttttcccctctctgttagatgaggatagcaaaataaagttttttaaggggaaaaagctcaatattgtagca
ttacgatctctggaggattgtataaattacgtgactaaatccgagaatatgatagaaatgatgaaggaaagatcgacta
ttttaaatagcatagatatagaaacggaatcggtagatcgtctaaaagaattgcttctaaaatgaaaaaaaacactgat
tcagaaatggatcaacgactagggtataagtttttggtgcctgatcctaaagccggagttttttatagaccgttacatttcc
aatatgtatcgtattctaattttatattgcatcgattgcatgaaatcttgaccgtcaagcggccactcttatcgtttaagaata
atacagaacgaattatgatagaaattagcaatgttaaagtgactcctccagattactcacctataatcgcgagtattaaa
ggtaagagttatgatgcattagccacgttcactgtaaatatctttttttctagacactaaataaaatatttaaaatataatatt
aatgtactaaaacttatatattattaatttatctaactaaagttagtaaattatatatataattttataattaatttaattttactaat
tttatttagtgtctagaaaaaaa

An example sequence of sVAC fragment 3 (F3, 66679 bp in length) (SEQ ID NO: 18):

tttttttctagacactaaataaaatatttaaaatataatattaatgtactaaaacttatatattattaatttatctaactaaagtta
gtaaattatatatataattttataattaatttaattttactaattttatttagtgtctagaaaaaaaattttgggagcgtctttatttat
tcgaagggtaaaaatatggtagaacttggatctggaaactcttttcaaataccagatgatatgcgaagtgcgtgtaaca
aagtattagacagcgataacggaatagactttctgagatttgttttgttaaacaatagatggataatggaagatgctatat
caaaatatcagtctccagttaatatatttaaactagctagtgagtacggattaaacatacccaaatatttagaaattgaa
atagaggaagacacattatttgacgacgagttatactctattatagaacgctcttttgatgataaatttccaaaaatatcc
atatcgtatattaagttgggagaacttaggcggcaagttgtagactttttcaaattctcgttcatgtatattgagtccatcaa
ggtagatcgtataggagataatatttttattcctagcgttataacaaaatcaggaaaaaagatattagtaaaagatgtag
accatttaatacgatctaaggttagagaacatacatttgtaaaagtaaaaaagaaaaacacattttccattttatacgac
tatgatgggaacggaacagaaactagaggagaagtaataaaacgaattatagacactataggacgagactattat
gttaacggaaagtatttctctaaggttggtagtgcaggcttaaagcaattgactaataaattagatattaatgagtgcgca
actgtcgatgagttagttgatgagattaataaatccggaactgtaaaacgaaaaataaaaaaccaatcagcatttgatt
taagcagagaatgtttgggatatccagaagcagattttataacgttagttaataacatgcggttcaaaatagaaaattgt
aaggttgtaaatttcaatattgaaaatactaattgtttaaataacccgagtattgaaactatatatggaaactttaaccagtt
cgtctcaatctttaatatcgtcaccgatgtcaaaaaaagattattcgagtgaaataatatgcgcctttgatataggtgcaa
aaaatcctgccagaactgttttagaagtcaaggataactccgttagggtattggatatatcaaaattagactggagttct
gattgggaaaggcgcatagctaaagatttgtcacaatatgaatacactacagttcttctagaacgtcagcctagaaggt
cgccgtatgttaaatttatctattttattaaaggctttttatatcatacatcggctgccaaagttatttgcgtctcgcctgtcatgt
ctggtaattcatatagagatcgaaaaaagagatcggtcgaagcatttcttgattggatggacacattcggattgcgaga
ctccgttccggatagacgcaaattagacgatgtagcggatagtttcaatttggctatgagatacgtattagataaatgga
atactaattatacaccttataataggtgtaaatctagaaattacataaaaaaaatgtaataacgttagtaacgccattatg
gataatctatttacctttctacatgaaatagaagatagatatgccagaactatttttaactttcatctaataagttgcgatga
aataggagatatatatggtcttatgaaagaacgcatttcctcagaggatatgtttgataatatagtatataataaagatat
acatcctgccattaagaaactagtgtattgcgacatccaacttactaaacacattattaatcagaatacgtatccggtatt
taacgattcttcacaagtgaaatgttgtcattatttcgacataaactcagataatagcaatattagctctcgtacagtagag
atatttgagagggaaaagtcatctcttgtatcatatattaaaactaccaataagaagagaaaggtcaattacggcgaa
ataaagaaaactgttcatggaggcactaatgcaaattacttttccggtaaaaagtctgacgagtatctgagtactacag
ttagatccaacattaatcaaccttggatcaaaaccatttctaagagaatgagagtagatatcattaatcactctatagtaa
cgcgtggaaaaagctctatattacaaactatagaaattatttttactaatagaacatgtgtgaaaatattcaaggattcta
ctatgcacattattctatccaaggacaaggatgaaaaggggtgtatacacatgattgacaaattattctatgtctattata
atttatttctgttgttcgaagatatcatccaaaacgagtactttaaagaagtagctaatgttgtaaaccacgtacttacggct
acggcattagatgagaaattattcctaattaagaaaatggctgaacacgatgtttatggagttagcaatttcaaaatagg
gatgtttaacctgacatttattaagtcgttggatcataccgttttcccctctctgttagatgaggatagcaaaataaagtttttt
aaggggaaaaagctcaatattgtagcattacgatctctggaggattgtataaattacgtgactaaatccgagaatatga
tagaaatgatgaaggaaagatcgactattttaaatagcatagatatagaaacggaatcggtagatcgtctaaaagaa
ttgcttctaaaatgaaaaaaaacactgattcagaaatggatcaacgactagggtataagtttttggtgcctgatcctaaa
gccggagttttttatagaccgttacatttccaatatgtatcgtattctaattttatattgcatcgattgcatgaaatcttgaccgt
caagcggccactcttatcgtttaagaataatacagaacgaattatgatagaaattagcaatgttaaagtgactcctcca
gattactcacctataatcgcgagtattaaaggtaagagttatgatgcattagccacgttcactgtaaatatctttaaagag
gtaatgaccaaagagggtatatccatcactaaaataagtagttatgagggaaaagattctcatttgataaaaattccgc
tactaataggatacgggaataaaaatccacttgatacagccaagtatcttgttcctaatgtcataggtggagtctttatca
ataaacaatctgtcgaaaaagtaggaattaatctagtagaaaagattacaacatggccaaaatttagggttgttaagc
caaactcattcactttctcgttttcctccgtatcccctcctaatgtattaccgacaagatatcgccattacaagatatctctgg
atatatcacaattggaagcgttgaatatatcatcgacaaagacatttataacggtcaatattgttttgctgtctcaatatttat
ctagagtgagtctagaattcattagacgtagtttatcatacgatatgcctccagaagttgtctatctagtaaacgcgataat
agatagtgctaaacgaattactgaatctattactgactttaatattgatacatacattaatgacctggtggaagctgaaca
cattaaacaaaaatctcagttaacgatcaacgagttcaaatatgaaatgctgcataactttttacctcatatgaactatac
acccgatcaactaaagggattttatatgatatctttactaagaaagtttctctactgtatctaccacacttctagatatccag
atagagattcgatggtttgtcatcgcatcctaacgtacggcaaatattttgagacgttggcacatgatgaattagagaatt
acataggcaacatccgaaacgatatcatgaacaatcacaagaacagaggcacttacgcggtaaacattcatgtact
aacaactcccggacttaatcacgcgttttctagcttattgagtggaaagttcaaaaagtcagacggtagttatcgaaca
catcctcactattcatggatgcagaatatttctattcctaggagtgttggattttatccggatcaagtaaagatttcaaagat
gttttctgtcagaaaataccatccaagtcaatatctttacttttgttcatcggacgttccggaaagaggtcctcaggtaggtt
tagtatctcaattgtctgtcttgagttccattacaaatatactaacgtctgagtatttggatttggaaaagaaaatttgtgagt
atatcagatcatattataaagatgatataagttactttgaaacaggatttccaatcactatagaaaatgctctagtcgcat
ctcttaatccaaatatgatatgtgattttgtaactgactttagacgtagaaaacggatgggattcttcggtaacttggaggt
aggtattactttagttagggatcacatgaatgaaattcgcattaatattggagcgggaagattagtcagaccattcttggtt
gtggataacggagagctcatgatggatgtgtgtccggagttagaaagcagattagacgacatgacattctctgacatt
cagaaagagtttccgcatgtcatcgaaatggtagatatagaacaatttacttttagtaacgtatgtgaatcggttcaaaa
atttagaatgatgtcaaaggatgaaagaaagcaatacgatttatgtgactttcctgccgaatttagagatggatatgtgg
catcttcattagtgggaatcaatcacaattctggacccagagctattcttggatgtgctcaagctaaacaagctatctcttg
tctgagttcggatatacgaaataaaatagacaatggaattcatttgatgtatccagagaggccaatcgtgattagtaag
gctttagaaacttcaaagattgcggctaattgcttcggccaacatgttactatagcattaatgtcgtacaaaggtatcaat
caagaggatggaattatcatcaaaaaacaatttattcagagaggcggtctcgatatagttaccgcaaagaaacatca
agtagaaattccgttggaaaactttaataacaaagaaagagataggtctaacgcctattcaaaattagaaagtaatgg
attagttagactgaatgctttcttggaatccggagacgctatggcacgaaatatctcatcaagaactcttgaagatgatttt
gctagagataatcagattagcttcgatgtttccgagaaatataccgatatgtacaaatctcgcgttgaacgagtacaagt
agaacttactgacaaagttaaggtacgagtattaaccatgaaagaaagaagacccattctaggagataaatttacca
ctagaacgagtcaaaagggaacagtcgcgtatgtcgcggatgaaacggaacttccatacgacgaaaatggtatca
caccagatgtcattattaattctacatccatcttctctagaaaaactatatctatgttgatagaggttattttaacagccgcat
attctgctaagccgtacaacaataagggagaaaaccgacctgtctgttttcctagtagtaacgaaacatccatcgatac
atatatgcaattcgctaaacaatgttatgagcattcaaatccgaaattgtccgatgaagaattatcggataaaatcttttgt
gaaaagattctctatgatcctgaaacggataagccttatgcatccaaagtattttttggaccaatttattacttgcgtctgag
gcatttaactcaggacaaggcaaccgttagatgtagaggtaaaaagacgaagctcattagacaggcgaatgaggg
acgaaaacgtggaggaggtatcaagttcggagaaatggagagagactgtttaatagcgcatggcgcagccaatac
tattacagaagttttaaaagactcagaagaggattatcaagatgtgtatgtttgtgaaaattgtggagacatagcagca
caaatcaagggtattaatacatgtcttagatgttcaaaacttaatctctctcctctcttaacaaaaattgataccacgcacg
tatctaaagtatttcttactcaaatgaacgccagaggcgtaaaagtcaaattagatttcgaacgaaggcctccttcgtttt
ataaaccattagataaagttgatctcaagccgtcttttctggtgtaatattctagtttggtagtagatacatatcaatatcatc
aaattcgagatccgaattataaaatgggcgtggattgttaactatagaatcggacgtctgatattcgaaaatctgtggag
ttttaggttttggtggaggtgtaactgctacttgggatactgaagtctgatattcagaaagctgggggatgttctggttcgac
atccaccgatggtgtcacatcactaatcggttcggtaacgtctgtggatggaggtgctacttctacagaacctgtagcct
cagttgtcaacggagatacatttttaatgcgaggaaatgtataatttggtaatggtttctcatgtggatctgaagaagagg
taagatatctactagaaagataccgatcacgttctagttctcttttgtagaacttaactttttctttctcagcatctagttgatatt
ccaacctcttcacgttactacgttcagattccaattcacgttcgcatgggttacctccgcagtttttacgagcgatttcacgtt
cagccttcatgcgtctctccttctctctatcgagtttatcagagcagtctttctgaaggcgatcgaactccataaatttctcca
acgctttgattgtttccatagatttccgaagttcagcttttaggactgtgattctttttctttcgaattcacagctggatgtgcaa
ccgtttccattaccgccatctctaagtttcttttctagatcggcaacatttcatccccatgccttttacattcctcgagtctactg
tcgtcgaaatatcgttccagctccttttcgacatcaataactttagcacgttgtctctcaagctctcttttgtagttatctgattc
cctggcacgtttaagatcttcatgcaattgagtcagctcttaacacaatctcttgcttcttcgtcatagtacttacaatcacta
tgggatccattgttaccacgtctacactcggcgagctcgcgtttaagagattcaatttcccgtttgtattggtccatgtttcca
ttgctaccaccattagatttacaggctgctagttgtcgttcgagatcagaaatacgggttttcttggaattgatttcgtcgatg
tacttggcatcgaaacacttattaagttctttttccaattctacgattttatttctttcgcgagtcaattccctcctgtagtaactat
ctgttttgtcagattcacgctctctacgtagactttcttgcaagttactaatttgttccctagcacgtccgagtttagttttatatg
ctgaatagagttctgattcatcctttgagcagatctctagcgatcgtttaagattcctgattctagtctttagcctatttacctcc
tcagaagatgttccgttaccgttgcgtttacactcgttaagctgtctatcaagatccatgattctatctctaagacgttgcatc
tctctttccatatcagcattgctttcattattacgtctgcagtcactcaactgtctttcaatatctgagattctatctctaagacgt
cgcatctctctctgtttcggcattggtttcattattacgtctacagtcgttcaactgtctttcaagatctgatattctagattggag
tctgctaatctctgtagcattttcacggcattcactcagttgtctttcaagatctgagattttagattggagtctgctaatctctg
taagatttcctcctccgctctcgatgcagtcggtcaacttattctctagttctctaatacgcgaacgcagtgcatcaacttctt
gcgtgtcttcctggttgcgtgtacattcatcgagtctagattcgagatctctaacgcgtcgtcgttcttcctcaagttctctgc
gtactacagaaagcgtgtccctatcttgttgatatttagcaatttctgattctagagtactgattttgcttacgtagttactaata
gttgtcttggccttatcaagatcctccttgtatttgtcgcattccttgatatccctacgaagtctggacagttcccattcgacat
tacgacgtttatcgatttcagctcggagatcgtcatcgcgttgttttagccacatacgactgagttcaagttctcgttgacaa
gatccatctacttttccattcctaatagtatccagttccttttctagttctgaacgcatttctcgttccctatcaagcgattctctc
aattctcggatagtcttcttatcaatttctaataaatctgaaccatcatctgtcccattttgaatatccctgtgttctttgatctcttt
tgtaagtcggtcgattctttcggttttataaacagaatccctttccaaagtcctaatcttactgagtttatcactaagttctgca
ttcaattcggtgagttttctcttggcttcttccaactctgttttaaactctccactatttccgcattcttcctcgcatttatctaacca
ttcaattagtttattaataactagttggtaatcagcgattcctatagccgttcttgtaattgtgggaacataattaggatcttct
aatggattgtatggcttgatagcatcatctttatcattattagggggatggacaaccttaattggttggtcctcatctcctcca
gtagcgtgtggttcttcaataccagtgttagtaataggcttaggcaaatgcttgtcgtacgcgggcacttcctcatccatca
agtatttataatcgggttctacgtctgaatattcttttctaagagacgcgacttcgggagttagtagaagaactctgtttctgt
atctatcaacgctggaatcaatactcaagttaaggatagcgaatacctcatcgtcatcatccgtatcttctgaaacacca
tcatatgacatttcatgaagtctaacgtattgataaatagaatcagatttagtattaaacagatccttaacctttttagtaaa
cgcatatgtatattttagatctccagatttcataatatgatcacatgccttaaatgtcagtgcttccatgatataatctggaac
actaatgggtgacgaaaaagatacagcaccatatgctacgttgataaataaatctgaaccactaagtagataatgatt
aatgttaaggaagaggaaatattcagtatatagatatgccttagcatcatatcttgtactaaacacgctaaacagtttatt
gatgtgatcaatttccaacagaataattagagcagcgggaataccaacaaacatattaccacatccgtattttctatga
atatcacatatcatgttaaaaaatcttgatagaagagcgaatatctcgtctgacttaatgagtcgtagttcagcagcaac
ataagtcataactgtaaatagaacatactttcctgtagtgttgattctagactccacatcaacaccattattaaaaatagttt
tatatacatctttaatctgctctccgttaatcgtcgaacgttctagtatacggaaacactttgatttcttatctgtagttaatgac
ttagtgatatcacgaagaatattacgaattacatttcttgtttttcttgagagacctgattcagaactcaactcatcgttccat
agtttttttacctcagtggcgaaatctttggagtgcttggtacatttttcaataaggttcgtgacctccatttattataaaaaatt
ttattcaaaacttaactacaatcgggtaattataaaatcgtagatctcccatgtggtggaatactaccatctatcgcatgtg
gatggacagtaggtaatggccatgggaacagtaatgtttgcatatttatctttcttgccagtattactgcatattgtcccaat
gtttcgatgtgatgttctaacctatcaactgccgctgtatcacaacaatagtgtccgatgaaattaagattatgatccaatg
tgtttaatatatgattatcaagtcttatacgatccgcgtcttttttgacaggatcaggttcttctacaggaagaagtttcggcct
cttatgatattcatgtctgggaaacggtggtctagggtgaggctccggtatcggagtgggttttggattataatcatcatcg
tctatgacatcatcttcgacttcgatatttattttgctatcttgatgatgtcctgtatcagttgcattttcagcactcgactgaata
ttagcgcattcattgtctattattaccatatttctaaacccaaaatgtatgtgttgaacatcagtactatcgttgatgagtcttat
agcatgaattcgcttatcgttatcgggtttatcttctgtcaccttagcaattccttttttattaaactctacataatcatatccattt
ctattgtttgttctaatataaacgagtatagcatcattgctaaatttttcaatagtatcgaaaacagaatatcctaaaccata
taatatatattcagggacactcaaactaaatgtccaggattctcctaaatacgtaaactttaatagtgcgaaatcattcaa
aaatctaccacttatagatagatagtacataaatgcgtatagtagtctacctatctctttattatgaaaaccggcattacga
tcatatatgtcgtgatatacctgtgatccgtttacgttaaaccataaatacatgggtgatcctataaacatgaatttatttcta
attctcagagctatagttaattgaccgtgtaatatttgcttacatgcatacttgatacgctcattaataaaatttttatcattgct
cgttatctcagaatcgtatatataaggagtaccatcgtgattcttaccagatattatacaaaatactatatataaaatatatt
gaccaacgttagtaatcatataaatgtttaacgttttaaattttgtattcaatgatccattatcatacgctagcatggtcttatg
atattcattctttaaaatataatattgtgttagccattgcattggggctcctaatggagattttttattctcatccattttaggata
ggctttcataaagtccctaataacttcgtgaataatgtttctatgttttctactgatgcatgtatttgcttcgatttttttatcccatg
tttcatctatcatagatttaaacgcagtaatgctcgcaacattaacatcttgaaccgttggtacaattccgttccataaattt
ataatgttcgccatttatataactcattttttgaatatacttttaattaacaaaagagttaagttactcatatgggcgccgtcca
gtctgaacatcaatctttttagccagagatatcatagccgctcttagagtttcagcgtgattttccaacctaaatagaacttc
atcgttgcgtttacaacacttttctatttgttcaaactttgttgttacattagtaatctttttttccaaattagttagccgttgtttgag
agtttcctcattgtcgtcttcatcggctttaacaattgcttcgcgtttagcctctggctttttagcagcctttgtagaaaaaaatt
cagttgctggaattgcaagatcgtcatctccggggaaaagagttccgtccatttaaagtacagattttagaaactgaca
ctctgcgttatttatatttggtacaacacatggattataaatatcgatgttaataacatcagaaaatgtaaagtctatacatt
gttgcatcgtgttaaattttctaatggatctagtattattgggtccaacttctgcctgaaatccaaatatggaagcggataca
aaaccgtttcctggataaaccacacatctccacttttgctttacatcagaaattgtgtcgttgacatcttgaactctcctatct
aatgccggtgttccacctatagattttgaatattcgaatgctgcatgagtagcattaaattccttaatattgccataattttcat
atattgagtaaccctggataaaaagtaaacacaccgcagccgtcgctaccacaataaaaaaaattgatagagagtt
catttataatctattagaagctgacaaaatttttttacacgcatcagacaatgctttaataaatagttcaacatctacttttgtc
atatcgaaccgatggtatgattctaacctagaattacatccgaaaaagttgactatgttcatagtcattaagtcattaaca
aacaacattccagactctggattataagacgatactgtttcgtcacaattacctaccttaatcatgtgattatgaatattgg
ctattagagcaccttctaagaaatctataatatctttgaaacacgatttaaaatcaaaccacgaatatacttctacgaag
aaagttagtttacccataggagaaataactataaatggagatctaaatacaaaatccggatctatgatagttttaacatt
attatattctctattaaatacctccacatctaaaaatgttaattttgaaactatgtcttcgtttattaccgtacctgaactaaac
gctataagctctattgtttgagaactctttaaacgatattcttgaaatacatgtaacaaagtttcctttaactcggtcggtttat
ctaccatagttacagaatttgtatccttatctataatataataatcaaaatcgtataaagttatataattatcgcgttcagatt
gggatcttttcaaatagactaaaaaccccatttctctagtaagtatcttatgtatatgtttgtaaaatatcttcatggtgggaa
tatgctctaccgcagttagccattcctcattgacagcggtagatgtattagacaaaactattccaatgtttaacaagggcc
attttacgagattattaaatccttgtttgataaatgtagccaatgagggttcgagttcaacgacgattgaattctcttcccgc
ggatgctgcatgatgaacgacgggatgttgttcgattgatttggaattctttttcgactttttgtttatattaaatattttaaaattt
atagcggatagcaattcatgtaccacggataatgtagacgcgtattgcgcatcgatatctttattattagataaatttatca
ataaatgtgagaagtttgcctcgttaaggtcttccatttaaatattatataaacatttgtgtttgtatcttattcgtcttttatggaa
tagttttttactagtaaagctgcaattacacactttgtccgtaaaacataaatataaacaccagcttttatcaatcgttccaa
aaagtcgacggcggacatttttaacatggcatctattttaaatacacttaggtttttggaaaaaacatcattttataattgta
acgattcaataactaaagaaaagattaagattaaacataagggaatgtcatttgtattttataagccaaagcattctacc
gttgttaaatacttgtctggaggaggtatatatcatgatgatttggttgtattggggaaggtaacaattaataatctaaagat
gatgctattttacatggatttatcatatcatggagtgacaagtagtggagcaatttacaaattgggatcgtctatcgataga
ctttctctaaataggactattgttacaaaagttaataattatgatgatacattttttgacgacgatgattgatcgctattgcac
aattttgtttttttactttctaatatagcgtttagattctttttcatgtgcgaatattgatttactaaaatatcgatgtttaacttttgttct
atgacgtccttatcagcggtatcggtacatatacgtaattcaccttcacaaaatacggagtcttcgataataatagccaa
tcgattattggatctagctgtctgtatcatattcaacatgtttaatatatcctttcgtttcccctttacaggcatcgatcgtagcat
attttccgcgtctgagatggaaatgttaaaactacaaaaatgcgtaatgttagcccgtcctaatattggtacgtgtctataa
gtttggcatagtagaataatagacgtgtttaaatgccttccaaagtttaagaattctattagagtattgcattttgatagtttat
cacctacatcatcaaaaataagtaaaaagtgtgctgattttttatgattttgtgcgacagcaatacatttttctatgttactttt
agttcgtatcagattatattctagagattcctgactactaacgaaattaatatgatttggccaaatgtatccatcataatctg
ggttataaacgggtgtaaacaagaatatatgtttatattttttaactagtgtagaaaacagagatagtaaatagatagttttt
ccagatccagatcctcccgttaaaaccattctaaacggcatttttaataaattttctcttgaaaattgtttttcttggaaacaa
ttcataattatatttacagttactaaattaatttgataataaatcaaaatatggaaaactaaggtcgttagtagggaggag
aacaaagaaggcacatcgtgacataaataacatttattatcatgatgacaccagaaaacgacgaagagcagacat
ctgtgttctccgctactgtttacggagacaaaattcaaggaaagaataaacgcaaacgcgtgattggtctatgtattaga
atatctatggttatttcactactatctatgattaccatgtccgcgtttctcatagtgcgcctaaatcaatgcatgtctgctaacg
aggctgctattactgacgccgctgttgccgttgctgctgcatcatctactcatagaaaggttgcgtctagcactacacaat
atgatcacaaagaaagctgtaatggtttatattaccagggttcttgttatatattacattcagactaccagttattctcggat
gctaaagcaaattgcactgcggaatcatcaacactacccaataaatccgatgtcttgattacctggctcattgattatgtt
gaggatacatggggatctgatggtaatccaattacaaaaactacatccgattatcaagattctgatgtatcacaagaa
gttagaaagtatttttgtgttaaaacaatgaactaatatttatttttgtacattaataaatgaaatcgcttaatagacaaactg
taagtaggtttaagaagttgtcggtgccggccgctataatgatgatactctcaaccattattagtggcataggaacatttct
gcattacaaagaagaactgatgcctagtgcttgcgccaatggatggatacaatacgataaacattgttatttagatact
aacattaaaatgtctacagataatgcggtttatcagtgtcgtaaattacgagccagattgcctagaccggatactagac
atctgagagtattgtttagtattttttataaagattattgggtaagtttaaaaaagaccaatgataaatggttagatattaata
atgataaagatatagatattagtaaattaacaaattttaaacaactaaacagtacgacggatgctgaagcgtgttatat
atacaagtctggaaaactggttaaaacagtatgtaaaagtactcaatctgtactatgtgttaaaaaattctacaagtgac
aacaaaaaatgaattaataataagtcgttaacgtacgccgccatggacgccgcgtttgttattactccaatgggtgtgtt
gactataacagatacattgtatgatgatctcgatatctcaatcatggactttataggaccatacattataggtaacataaa
aactgtccaaatagatgtacgggatataaaatattccgacatgcaaaaatgctactttagctataagggtaaaatagtt
cctcaggattctaatgatttggctagattcaacatttatagcatttgtgccgcatacagatcaaaaaataccatcatcata
gcatgcgactatgatatcatgttagatatagaagataaacatcagccattttatctattcccatctattgatgtttttaacgct
acaatcatagaagcgtataacctgtatacagctggagattatcatctaatcatcaatccttcagataatctgaaaatgaa
attgttgtttaattcttcattctgcatatcagacggcaatggatggatcataattgatgggaaatgcaatagtaattttttatca
taaaagttgtaaagtaaataataaaacaataaatattgaactagtagtacgtatattgagcaatcagaaatgatgctgg
tacctcttatcacggtgaccgtagttgcgggaacaatattagtatgttatatattatatatttgtaggaaaaagatacgtact
gtctataatgacaataaaattatcatgacaaaattaaaaaagataaagagttctaattccagcaaatctagtaaatcaa
ctgatagcgaatcagactgggaggatcactgtagtgctatggaacaaaacaatgacgtagataatatttctaggaatg
agatattggacgatgatagcttcgctggtagtttaatatgggataacgaatccaatgtcatggcgcctagcacagaaca
catttacgatagtgttgctggaagcacgctgctaataaataatgatcgtaatgaacagactatttatcagaacactacag
tagtaattaatgagacggagactgttgaagtacttaatgaagataccaaacagaatcctaactattcatccaatcctttc
gtaaattataataaaaccagtatttgtagcaagtcaaatccgttcattacagaactcaacaataaatttagtgagaataa
tccgtttagacgagcacatagcgatgattatcttaataagcaagaacaagatcatgaacacgatgatatagaatcatc
ggtcgtatcattggtgtgattagtttcctttttataaaattgaagtaatatttagtattattgctgccgtcacgttgtacaaatgg
agatattccctgtattcggcatttctaaaattagcaattttattgctaataatgactgtagatattatatagatacagaacatc
aaaaaattatatctgatgagatcaatagacagatggatgaaacggtacttcttaccaacatcttaagcgtagaagttgt
aaatgacaatgagatgtaccatcttattccccatagactatcgactattatactctgtattagttctgtcggaggatgtgttat
ctctatagataatgacatcaatgacaaaaatattctaacatttcccattgatcatgctgtaatcatatccccactgagtaa
atgtgtcgtagttagcaagggtcctacaaccatattggttgttaaagcggatatacccagcaaacgattggtaacatcgt
ttacaaacgacatactgtatgtaaacaatctgtcactgattaattatttgccgttgtctgtattcattattagacgagtcaccg
actatttggatagacacatatgcgatcagatatttgcgaataataagtggtattccattataaccatcgacgataagcaa
tatcctattccatcaaactgtataggtatgtcctctgccaagtacataaattctagcatcgagcaagatactttaatacatg
tttgtaacctcgagcatccattcgacttagtatacaaaaaaatgcagtcgtacaattctgtacctatcaaggaacaaata
ttgtacggtagaattgataatataaatatgagcattagtatttctgtggattaatagatttctagtatggggatcattaatcat
ctctaatctctaaatacctcataaaacgaaaaaaaagctattatcaaatactgtacggaatggattcattctcttctcttttt
atgaaactctgttgtatatctactgataaaactggaagcaaaaaatctgataaaaagaataagaataagatcaagga
ttatatggaacacgattattataaaataacaatagttcctggttcctcttccacgtctactagctcgtggtattatacacatg
cctagtaatagtctctttgcgttgacggaaagcagactagaaataacaggctaaaatgttcagacaccataatagttcc
caacccagataataacagagtaccatcaacacattcctttaaactcaatcccaaacccaaaaccgttaaaatgtatcc
ggccaattgatagtagataatgaggtgtacagcgcatgataatttacacagtaaccaaaatgaaaatactttagtaatt
ataagaaatatagatggtaacgtcatcatcaacaatccaataatatgccggagagtaaacattgacggataaaaca
aaaatgctccgcataactctatcatggcaataacacaaccaaatacttgtaagattcctaaattagtagaaaatacaa
cggatatcgatgtataagtgatctcgagaaataataagaataaagtaatgcccgtaaagataaacatcaacattgtttg
gtaatcattaaaccaattagtatgaagttgaactaatttcacagtagattttattccagtgttatcctcgcatgtatacgtacc
tggtaagatatctttatattctataatcaatgagacatcactatccgataacgaatgaagtctagcactagtatgccattta
cttaatatggtcgtcttggaagttttattataagttaaaatatcatggttgtccaatttccatctaatatactttgtcggattatct
atagtacacggaataatgatggtattattacatgctgtatactctatagtctttgtagatgttataatcataaaagtacagag
gtatatcaacgatattctaactcttgacattttttatttatttaaaatgatacctttgttatttattttattctattttgctaacggtatc
gaatggcataagtttgaaacgagtgaagaaataatttctacttacttattagacgacgtattatacacgggtgttaatggg
gcggtatacacattttcaaataataaactaaacaaaactggtttagctaatactaattatatcacaacatctataaaagt
agaggatgcggataaggatacattagtatgcggaaccaataacggaaatcccaaatgttggaaaatagacggttca
gacgacccaaaacatagaggtagaggatacgctccttatcaaaatagcaaagtaacgataatcagtcacaacgga
tgtgtactatctgacataaacatatcaaaagaaggaattaaacgatggagaagatttgacggaccatgtggttatgattt
atacacggcggataacgtaattccaaaagatggtttacgaggagcattcgtcgataaagatggtacttatgacaaagt
ttacattcttttcactgatactatcggctcaaagagaattgtcaaaattccgtatatagcacaaatgtgcctaaacgacga
aggtggtccatcatcattgtctagtcatagatggtcgacgtttctcaaagtcgaattagaatgtgatatcgacggaagaa
gttatagacaaattattcattctagaactataaaaacagataatgatacgatactatatgtattcttcgatagtccgcattat
gtacctattctatgaataccattaaacaatctttttctacgtcaaaattggaaggatatacaaagcaattgccgtctccag
ctcctggtatatgtctaccagctggaaaagttgttccacataccacgtttgaagtcatagaaaaatataatgtactagatg
atattataaagcctttatctaaccaacctatcttcgaaggaccgtctggtgttaaatggttcgatataaaggagaaggaa
aatgaacatcgggaatatagaatatacttcataaaagaaaattctatatattcgttcgatacaaaatctaaacaaactc
gtagctcgcaagtcgatgcgcgactattttcagtaatggtaacttcgaaaccgttatttatagcagatatagggatagga
gtaggaatgccacaaatgaaaaaaatacttaaaatgtaatcttaatcgagtacaccacacgacaatgaacaaacat
aagacagattatgctggttatgcttgctgcgtaatatgcggtctaattgttggaattatttttacagcgacactattaaaagtt
gtagaacgtaaattagttcatacaccatcaatagataaaacgataaaagatgcatatattagagaagattgtcctactg
actggataagctataataataaatgtatccatttatctactgatcgaaaaacctgggaggaaggacgtaatgcatgca
aagctctaaatccaaattcggatctaattaagatagagactccaaacgagttaagttttttaagaagcattagacgcgg
atattgggtaggagaatccgaaatattaaaccagacaaccccatataattttatagctaagaatgccacgaagaatg
gaactaaaaaacggaaatatatttgtagcacaacgaatactcccaaactgcattcgtgttacactatataacaattaca
ctacatttttatcataccactacttcggttagatgttttagaaaaaaataaatatcgccgtaccgttcttgtttttataaaaata
acaattaacaattatcaaattttttctttaatattttacgtggttgaccattcttggtggtaaaataatctcttagtgttggaatgg
aatgctgtttaatgtttccacactcatcgtatattttgacgtatgtagtcacatcgtttacgcaatagtcagactgtagttctat
catgcttcctacattagaaggaggaacagttttaaagtctcttggttttaatctattaccgttagttttcatgaaatcctttgtttt
atccacttcacattttaaataaatgtccactatacattcttctgttaattttactagatcgtcatgggtcatagaatttataggtt
ccgtagtccatggatccaaactagcaaacttcgcgtatacggtatcgcgattagtgtatacaccaactgtatgaaaatt
aagaaaacagtttaatagatcaacagaaatatttaatcctccgtttgatacagatgcaccatatttatggattttggattca
cacgttgtttgtctgaggggttcgtctagcgttgcttctacataaacttctattcccatatattctttattgtcagaatcgcatac
cgatttatcatcatacactgtttgaaaactaaatggtatacacatcaaaataacaaatactaacgagtacattctgcaat
attgttatcgtaattggaaaaatagtgttcgagtgagttggattatgtgagtattggattgtatattttattttatattttgtaataa
gaataaaatgctaatgtcaagtttattccaatagatgtcttattaaaaacatatataataaataacaatggctgaatggca
taaaattatcgaggatatctcaaaaaataataagttcgaggatgccgccatcgttgattacaagactacaaagaatgtt
ctagctgctattcctaacagaacatttgccaagattaatccgggtgaaattattcctctcatcactaatcgtaatattctaaa
acctcttattggtcagaaatattgtattgtatatactaactctctaatggatgagaacacgtatgctatggagttgcttactg
ggtacgcccctgtatctccgatcgttatagcgagaactcataccgcacttatatttttgatgggtaagccaacaacatcc
agacgtgacgtgtatagaacgtgtagagatcacgctacccgtgtacgcgcaactggtaattaaaataaaaagtaata
ttcatatgtagtgtcaattttaaatgatgatgatgaaatggataatatccatattgacgatgtcaataatgccggtattggca
tacagctcatcgatttttagatttcattcagaggatgtggaattatgttatgggcatttgtattttgataggatctataatgtagt
aaatataaaatataatccgcatattccatatagatataattttattaatcgcacgttaaccgtagatgaactagacgataa
tgtcttttttacacatggttattttttaaaacacaaatatggttcacttaatcctagtttgattgtctcattatcaggaaacttaaa
atataatgatatacaatgctcagtaaatgtatcgtgtctcattaaaaatttggcaacgagtacatctactatattaacatct
aaacataagacttattctctacatcggtccacgtgtattactataataggatacgattctattatatggtataaagatataa
atgacaagtataatggcatctatgattttactgcaatatgtatgctaatagcgtctacattgatagtgaccatatacgtgttt
aaaaaaataaaaatgaactcttaattatgctatgctattagaaatggataaaatcaaaattacggttgattcaaaaattg
gtaatgttgttaccatatcgtataacttggaaaagataactattgatgtcacacctaaaaagaaaaaagaaaaggatgt
attattagcgcaatcagttgctgtcgaagaggcaaaagatgtcaaggtagaagaaaaaaatattatcgatattgaag
atgacgatgatatggatgtagaaagcgcataatacgatctataaaaataagtatataaatactttttatttactgtactctta
ctgtgtagtggtgataccctactcgattatttttttaaaaaaatacttattctgattcttctagccatttccgtgttcgttcgaatg
ccacatcgacgttaaagataggggagtagttgaaatctagttctgcattgttggtacgcacctcaaatgtagtgttggat
atcttcaacgtatagttgttgagtagtgatggttttctaaatagaattctcttcatatcattcttgcacgcgtacatttttagcatc
catcttggaattctagatccttgttctattcccaatggtttcatcaatagaagattaaacatatcgtacgaacacgatggag
agtaatcgtagcaaaagtaagcatttcctttaatctcagatcccggatactggatatattttgcagccaacacgtgcatcc
atgcaacatttcctacatatacccggctatgcaccgcgtcatcatcgactgtacgatacataatgttaccgtgttgcttac
attgctcgtaaaagactttcgtcaatttgtctccttctccgtaaattccagtgggtcttaggcaacaagtatacaattttgctc
cattcatgattacggaattattggctttcataaccagttgctcggccatacgtttactttttgcgtatacatgtcctggtgatat
atcataaagggtatgctcatggccgatgaatggatcaccgtgtttatttggtcctattgcttccatgctactagtatagatca
aatacttgattcctaggtccacacaagctgccaatatagtctgtgttccataatagtttactttcatgatttcattatcggtgta
ttttccaaatacatccactagagcagccgtatgaataatcagatttaccccatctagcgcttctctcaccttatcaaagtcg
tttatatcacattgtatatagtttataaccttaactttcgaggttattggttgtggatcttctacaatatctatgactctgatttcttg
aacatcatctgcactaattaacagttttactatatacctgcctagaaatccggcaccaccagtaaccgcgtacacggcc
attgctgccactcataatatcagactacttattctattttactaaataatggctgtttgtataatagaccacgataatatcaga
ggagttatttactttgaaccagtccatggaaaagataaagttttaggatcagttattggattaaaatccggaacgtatagtt
tgataattcatcgttacggagatattagtcaaggatgtgattccataggcagtccagaaatatttatcggtaacatctttgt
aaacagatatggtgtagcatatgtttatttagatacagatgtaaatatatctacaattattggaaaggcgttatctatttcaa
aaaatgatcagagattagcgtgtggagttattggtatttcttacataaatgaaaagataatacattttcttacaattaacga
gaatggcgtttgatatatcagttaatgcgtctaaaacaataaatgcattagtttacttttctactcagcaaaataaattagtc
atacgtaatgaagttaatgatacacactacactgtcgaatttgatagggacaaagtagttgacacgtttatttcatataat
agacataatgacaccatagagataagaggggtgcttccagaggaaactaatattggttgcgcggttaatacgccggt
tagtatgacttacttgtataataagtatagttttaaactgattttagcagaatatataagacacagaaatactatatccggc
aatatttattcggcattgatgacactagatgatttggctattaaacagtatggagacattgatctattatttaatgagaaactt
aaagtagactccgattcgggactatttgactttgtcaactttgtaaaggatatgatatgttgtgattctagaatagtagtagc
tctatctagtctagtatctaaacattgggaattgacaaataaaaagtataggtgtatggcattagccgaacatatatctga
tagtattccaatatctgagctatctagactacgatacaatctatgtaagtatctacgcggacacactgagagcatagag
gataaatttgattattttgaagacgatgattcgtctacatgttctgccgtaaccgacagggaaacggatgtataatttttttta
tagcgtgaaggatatgataaaaaatataattgttgtatttatcccattccaatcaccttatatgattctgtaacacaatgaa
ggagtctcatagatgtatagaggtcagatactggtttgataaactgtttattccacatgagtatgtttgactttatggttagac
ccgcatactttaacaaatcactgaaaattggagttaggtattgacctctcagaatcagttgccgttctggaacattaaatg
tattttttatgatatactccaacgcatttatgtgggcatacaacaagtcattactaatggagtattccaagagttttagttgtct
agtatttaacaagagaagagatttcaacagactgtttatgaactcgaatgccgcctcattgtcgcttatattgatgatgtcg
aattctcccaatatcatcaccgatgagtagctcatcttgttatcgggatccaagttttctaaagatgtcattaaaccctcgat
catgaatggatttatcatcatcgtttttatgttggacatgagcttagtccgtttgtccacatctatagacgacgatttctgaatt
attttatatatccctctctttaactccaggaacttgtcaggatggtctactttaatatgttctcgtctaagagatgaaaatctttg
gatggttgcacgcgacttttctctaaaggatgacgttgcccaagatcctctcttaaatgaatccatcttatccttggacaag
atggacagtctattttccttagatggtttaatatttttgttacccatgatctataaaggtagacctaatcgtctcggatgaccta
tatatttattttcagttttattatacgcataaattgtaaaaaatatgttaggtttacaaaaatgtctcgtggggcattaatcgtttt
tgaaggattggacaaatctggaaaaacaacacaatgtatgaacatcatggaatctataccggcaaacacgataaa
atatcttaactttcctcagagatccactgtcactggaaagatgatagatgactatctaactcgtaaaaaaacctataatg
atcatatagttaatctattattttgtgcaaatagatgggagtttgcatcttttatacaagaacaactagaacagggaattact
ttaatagttgatagatacgcgttctctggagtagcgtatgccgccgctaaaggcgcgtcaatgactctcagtaagagtta
tgaatctggattgcctaaacccgacttagttatattcttggaatctggtagcaaagaaattaatagaaacgtcggcgag
gaaatttatgaagatgttacattccaacaaaaggtattacaagaatataaaaaaatgattgaagaaggagatattcatt
ggcaaattatttcttctgaattcgaggaagatgtaaagaaggagttgattaagaatatagttatagaggctatacacac
ggttactggaccagtggggcaactgtggatgtaatagtgaaattacattttttataaatagatgttagtacagtgttataaa
tggatgaagcatattactctggcaacttggaatcagtactcggatacgtgtccgatatgcataccgaactcgcatcaat
atctcaattagttattgccaagatagaaactatagataatgatatattaaacaaggacattgtaaattttatcatgtgtaga
tcaaacttggataatccatttatctctttcctagatactgtatatactattatagatcaagagaactatcagaccgaattgatt
aattcattagacgacaatgaaattatcgattgtatagttaataagtttatgagcttttataaggataacctagaaaatatag
tagatgctatcattactctaaaatatataatgaataatccagattttaaaactacgtatgccgaagtactcggttccagaat
agccgatatagatattaaacaagtgatacgtgagaatatactacaattgtctaatgatatccgcgaacgatatttgtgaa
aaatattaaaaaaaaatactttttttattaaatgacgtcgcttcgcgaatttagaaaattatgctgtgatatatatcacgcat
caggatataaagaaaaatctaaattaattagagactttataacagatagggatgataaatatttgatcattaagctattg
cttcccggattagacgatagaatttataacatgaacgataaacaaattataaaattatatagtataatatttaaacaatct
caggaagatatgctacaagatttaggatacggatatataggagacactattaggactttcttcaaagagaacacaga
aatccgtccacgagataaaagcattttaactttagaagacgtggatagtttcttaactacgttatcatccgtaactaaaga
atcgcatcaaataaaattattgactgatatcgcatccgtttgtacatgtaatgatttaaaatgtgtagtcatgcttattgataa
agatctaaaaattaaagcgggtcctcggtacgtacttaacgctattagtcctaatgcctatgatgtgtttagaaaatctaat
aacttgaaagagataatagaaaatgcatctaaacaaaatctagactctatatctatttctgttatgactccaattaatccc
atgttagcggaatcgtgtgattctgtcaataaggcgtttaaaaaatttccatcaggaatgtttgcggaagtcaaatacgat
ggtgaaagagtacaagttcataaaaataataacgagtttgccttctttagtagaaacatgaaaccagtactctctcata
aagtggattatctcaaagaatacataccgaaagcatttaaaaaagctacgtctatcgtattggattctgaaattgttcttgt
agacgaacataatgtaccgctcccgtttggaagtttaggaatacacaaaaagaaagaatataaaaactctaacatgt
gtttgttcgtgtttgactgtttgtactttgatggattcgatatgacggacattccattgtacgaacgaagatcttttctcaaaga
tgttatggttgaaatacccaatagaatagtattctcagagttgacgaatattagtaacgagtctcagttaactgacgtattg
gatgatgcactaacgagaaaattagaaggattggtcttaaaagatattaatggagtatacgaaccgggaaagagaa
gatggttaaaaataaagcgagactatttgaacgagggttccatggcagattctgccgatttagtagtactaggtgcttac
tatggtaaaggagcaaagggtggtatcatggcagtctttctaatgggttgttacgacgatgaatccggtaaatggaaga
cggttaccaagtgttcaggacacgatgataatacgttaagggttttgcaagaccaattaacgatggttaaaattaacaa
ggatcccaaaaaaattccagagtggttggtagttaataaaatctatattcccgattttgtagtagaggatccgaaacaat
ctcagatatgggaaatttcaggagcagagtttacatcttccaagtcccataccgcaaatggaatatccattagatttcct
agatttactaggattagagaagataaaacgtggaaagaatctactcatctaaacgatttagtaaacttgactaaatctta
atagttacatacaaactgaaaattaaaataacactatttagttggtggtcgccatggatggtgttattgtatactgtctaaa
cgcgttagtaaaacatggcgaggaaataaatcatataaaaaatgatttcatgattaaaccatgttgtgaaagagtttgt
gaaaaagtcaagaacgttcacattggcggacaatctaaaaacaatacagtgattgcagatttgccatatatggataat
gcggtatccgatgtatgcaattcactgtataaaaagaatgtatcaagaatatccagatttgctaatttgataaagatagat
gacgatgacaagactcctactggtgtatataattattttaaacctaaagatgttattcctgttatcatatctataggaaagg
ataaagatgtctgtgaactattaatctcatcagacatatcgtgtgcatgcgtggagttaaattcatatcacgtagccattctt
cccatggatgtttccttttttaccaaaggaaatgcatcattgattattctcctgtttgatttctctatcgatgcagcacctctctta
agaagtgtaaccgataataatgttattatatctagacaccagcgtctacatgacgagcttccgagttccaattggttcaa
gttttacataagtataaagtccgactattgttctatattatatatggttgttgatggatctgtgatgcatgcgatagctgataat
agaactcacgcaattattagcaaaaatatattagacaatactacgattaacgatgagtgtagatgctgttattttgaacc
acagattaggattcttgatagagatgagatgctcaatggatcatcgtgtgatatgaacagacattgtattatgatgaattt
acctgatgtaggcaaatttggatctagtatgttggggaaatatgaacctgacatgattaagattgctctttcggtggctggt
aatttaataagaaatcgagactacattcccgggagacgaggatatagctactacgtttacggtatagcctctagataatt
tttttaagcacgaaataaaaaacataattttaaaccaatctatttcatactattttgtgtgatcaccatggacataaagata
gatattagtatttctggtgataaatttacggtgactactaggagggaaaatgaagaaagaaaaaaatatctacctctcc
aaaaagaaaaaactactgatgttatcaaacctgattatcttgagtacgatgacttgttagatagagatgagatgtttacta
ttctagaggaatattttatgtacagaggtctattaggcctcagaataaaatatggacgactctttaacgaaattaaaaaat
tcgacaatgatgcggaagaacaattcggtactatagaagaactcaagcagaaacttagattaaattctgaagaggg
agcagataactttatagattatataaaggtacaaaaacaggatatcgtcaaacttactgtatacgattgcatatctatgat
aggattgtgtgcatgcgtggtagatgtttggagaaatgagaaactgttttctagatggaaatattgtttacgagctattaaa
ctgtttattaatgatcacatgcttgataagataaaatctatactgcagaatagactagtatatgtggaaatgtcatagaaa
gttaaaagttaatgagagcaaaaatatataaggttgtattccatatttgttattttttctgtaatagttagaaaaatacattcg
atggtctatctaccagattattatgtgttataaggtactttttctcataataaactagagtatgagtaagatagtgtttttcaaa
acatataaatctaaaattgatggatgagatatacagctattaatttcgaaaatatattttaatctgataactttaaacatgg
atttttgatggtggtttaacgttttaaaaaaagattttgttattgtagtatatgataatattaaaagatggatataaagaatttg
ctgactgcatgtactattttttacattactacattggctacggcagatatacctactccgccaccaacgggtcatgtgaca
agggagaatatcttgataagaggcataatcaatgttgtaatcggtgtccacctggagaatttgccaaggttagatgtaat
ggtaacgataacacaaaatgtgaacgctgcccacctcatacatataccacaatcccaattattctaatggatgtcatca
atgtagaaaatgcccaaccggatcatttgataaggtaaagtgtaccggaacacagaacagtaaatgttcgtgtcttcct
ggttggtattgtgctactgattcttcacagactgaagattgttgaaattgtgtaccaaaaaggagatgtccatgcggatac
tttggtggaatagatgaacaaggaaatcctatttgtaaatcgtgttgtgttggtgaatattgcgactacctacgtaattatag
acttgatccatttcctccatgcaaactatctaaatgtaattaattatgattttgatgataatgttaccatacattatatcgctact
tggttagtgtattattcagtatgaagacctattaataattacttatcttttgacgatcttgttataattataatataaaaatactta
tggcatagtaactcataattgctgacgcgataaattcgtaataatctgttttgttcaaatttttataaggaatctacaggcat
aaaaataaaaatataatttataatatactcttacagcgcgccatcatgaataacagcagtgaattgattgctgttattaat
ggatttagaaatagtggacgattttgtgatattagtatagttattaatgatgaaaggataaacgctcataaactcatcctat
ctggagcctccgaatatttttccattctgttttccaataattttatcgattctaatgaatacgaagttaatctaagtcatttagatt
atcaaagcgttaacgatttgatcgattacatttatgggatacctttgagcctaactaacgataacgtgaaatatattctttca
accgctgattttttacaaattggatctgctattacggagtgtgaaaattacatacttaaaaatctttgttctaaaaactgtatc
gatttctacatatacgctgataaatataataacaagaaaatagaatcagcgtcgtttaacacaatattacaaaatattttg
agactcatcaacgatgaaaactttaaatacttaacagaggaatcaatgataaaaattttaagcgatgatatgttaaatat
aaaaaatgaggattttgcaccactaattctcattaaatggttagagagtactcaacaatcatgcaccgtcgagttactta
gatgcctcagaatatcattgctttccccacaagttataaaatcactttatagtcatcaactggttagttcaatctacgaatgt
ataacattcttaaacaatatagcattcttggatgaatcatttcctagataccatagcatcgagttgatatctatcggtataag
taattcgcatgataagatttccataaactgctacaatcataaaaaaaatacatgggaaatgatatcttcacgtagatata
ggtgtagtttcgcagtggccgtcctggataatattatctatatgatgggtggatatgatcagtccccgtatagaagttcaa
aggttatagcgtacaatacatgtacaaattcttggatatatgatataccagagctaaaatatcctcgttctaattgtgggg
gactggctgatgacgaatacatttattgtataggcggcatacgcgatcaggattcatcgttgacatctagtattgataaat
ggaagccatcaaaaccatattggcagaagtatgctaaaatgcgcgaaccaaaatgtgatatgggggttgcgatgtta
aacggattaatatatgtcatgggtggaatcgttaaaggtgacacgtgtaccgacgcactagagagtttatcagaagat
ggatggatgaagcatcaacgtcttccaataaaaatgtccaatatgtcgacgattgttcatgatggcaagatttatatatct
ggaggttacaacaatagtagtgtagttaatgtaatatcgaatctagtccttagctataatccgatatatgatgaatggacc
aaattatcatcattaaacattcctagaattaatcccgctctatggtcagcgcataataaattatatgtaggaggaggaat
atctgatgatgttcgaactaatacatctgaaacatacgataaagaaaaagattgttggacattggataatggtcacgtgt
taccacgcaattatataatgtataaatgcgaaccgattaaacataaatatccattggaaaaaacacagtacacgaatg
attttctaaagtatttggaaagttttataggtagttgatagaacaaaatacataattttgtaaaaataaatcactttttatacta
atatgacacgattaccaatacttttgttactaatatcattagtatacgctacaccttttcctcagacatctaaaaaaataggt
gatgatgcaactctatcatgtaatcgaaataatacaaatgactacgttgttatgagtgcttggtataaggagcccaattcc
attattcttttagctgctaaaagcgacgtcttgtattttgataattataccaaggataaaatatcttacgactctccatacgat
gatctagttacaactatcacaattaaatcattgactgctagagatgccggtacttatgtatgtgcattctttatgacatcaac
tacaaatgacactgataaagtagattatgaagaatactccacagagttgattgtaaatacagatagtgaatcgactata
gacataatactatctggatctacacattcaccggaaactagttctaagaaacctgattatatagataattctaattgctcgt
cggtattcgaaatcgcgactccggaaccaattactgataatgtagaagatcatacagacaccgtcacatacactagtg
atagcattaatacagtaagtgcatcatctggagaatccacaacagacgagactccggaaccaattactgataaaga
agatcatacagttacagacactgtctcatacactacagtaagtacatcatctggaattgtcactactaaatcaaccacc
gatgatgcggatctttatgatacgtacaatgataatgatacagtaccaccaactactgtaggcggtagtacaacctctat
tagcaattataaaaccaaggactttgtagaaatatttggtattaccgcattaattatattgtcggccgtggcaattttctgtat
tacatattatatatataataaacgttcacgtaaatacaaaacagagaacaaagtctagatttttgacttacataaatgtct
gggatagtaaaatctatcatattgagcggaccatctggtttaggaaagacagccatagccaaaagactatgggaata
tatttggatttgtggtgtcccataccactagatttcctcgtcctatggaacgagaaggtgttgattaccattacgttaacaga
gaggccatctggaagggaatagccgccggaaactttctagaacatactgagtttttaggaaatatttacggaacttcta
aaacagctgtgaatacagcggctattaataatcgtatttgtgtgatggatctaaacatcgacggtgttagaagtcttaaa
aatacgtacctaatgccttactcggtgtatataagacctacctctcttaaaatggttgagaccaagcttcgttgtagaaac
actgaagctaacgatgagattcatcgtcgcgtgatattggcaaaaacggatatggatgaggccaacgaagcaggtct
attcgacactattatcattgaagatgatgtgaatttagcatatagtaagttaattcagatactacaggaccgtattagaatg
tattttaacactaattagagacttaagacttaaaacttgataattaataatataactcgtttttatatgtggctatttcaacgtct
aatgtattagttaaatattaaaacttaccacgtaaaacttaaaatttaaaatgatatttcattgacagatagatcacacatt
atgaactttcaaggacttgtgttaactgacaattgcaaaaatcaatgggtcgttggaccattaataggaaaaggtggatt
cggtagtatttatactactaatgacaataattatgtagtaaaaatagagcccaaagctaacggatcattatttaccgaac
aggcattttatactagagtacttaaaccatccgttatcgaagaatggaaaaaatctcacaatataaagcacgtaggtctt
atcacgtgcaaggcatttggtctatacaaatccattaatgtggaatatcgattcttggtaattaatagattaggtgcagatc
tagatgcggtgatcagagccaataataatagattaccaaaaaggtcggtgatgttgatcggaatcgaaatcttaaata
ccatacaatttatgcacgagcaaggatattctcacggagatattaaagcgagtaatatagtcttggatcaaatagataa
gaataaattatatctagtggattacggattggtttctaaattcatgtctaatggagaacatgttccatttataagaaatccaa
ataaaatggataacggtactctagaatttacacctatagattcgcataaaggatacgttgtatctagacgtggagatcta
gaaacacttggatattgtatgattagatggttgggaggtatcttgccatggactaagatatctgaaacaaagaattgtgc
attagtaagtgccacaaaacagaaatatgttaacaatactgcgactttgttaatgaccagtttgcaatatgcacctagag
aattgctgcaatatattaccatggtaaactctttgacatattttgaggaacccaattatgacgagtttcggcacatattaatg
cagggtgtatattattaagtgtggtgtttggtcgatgtaaaatttttgtcgataaaaattaaaaaataacttaatttattattgat
ctcgtgtgtacaaccgaaatcatggcgatgttttacgcacacgctctcggtgggtacgacgagaatcttcatgcctttcct
ggaatatcatcgactgttgccaatgatgtcaggaaatattctgttgtgtcagtttataataacaagtatgacattgtaaaag
acaaatatatgtggtgttacagtcaggtgaacaagagatatattggagcactgctgcctatgtttgagtgcaatgaatat
ctacaaattggagatccgatccatgatcaagaaggaaatcaaatctctatcatcacatatcgccacaaaaactactat
gctctaagcggaatcgggtacgagagtctagacttgtgtttggaaggagtagggattcatcatcacgtacttgaaaca
ggaaacgctgtatatggaaaagttcaacatgattattctactatcaaagagaaggccaaagaaatgaatgcacttagt
ccaggacctatcattgattaccacgtctggataggagattgtatctgtcaagttactgctgtggacgtacatggaaagga
aattatgagaatgagattcaaaaagggtgcggtgcttccgatcccaaatctggtaaaagttaaacttggggagaatga
tacagaaaatctttcttctactatatcggcggcaccatcgaggtaaccacctctctggaagacagcgtgaataatgtact
catgaaacgtttggaaactatacgccatatgtggtctgtcgtatatgatcattttgatattgtgaatggtaaagaatgctgtt
atgtgcatacgcatttgtctaatcaaaatcttataccgagtactgtaaaaacaaatttgtacatgaagactatgggatcat
gcattcaaatggattccatggaagctctagagtatcttagcgaactgaaggaatcaggtggatggagtcccagacca
gaaatgcaggaatttgaatatccagatggagtggaagacactgaatcaattgagagattggtagaggagttcttcaat
agatcagaacttcaggctggtgaatcagtcaaatttggtaattctattaatgttaaacatacatctgtttcagctaagcaac
taagaacacgtatacggcagcagcttcctttatactctcatcttttaccaacacaaagggtggatatttgttcattggagtt
gataataatacacacaaagtaattggattcacggtgggtcatgactacctcagactggtagagaatgatatagaaaa
gcatatcaaaagacttcgtgttgtgcatttctgtgagaagaaagaggacatcaagtacacgtgtcgattcatcaaggta
tataaacctggggatgaggctacctcgacatacgtgtgcgctatcaaagtggaaagatgctgttgtgctgtgtttgcaga
ttggccagaatcatggtatatggatactaatggtatcaagaagtattctccagatgaatgggtgtcacatataaaatttta
attaatgtaatagagaacaaataataaggttgtaatatcatatagacaataactaacaattaattagtaactgttatctctt
ttttaactaaccaactaactatatacctattaatacatcgtaattatagttcttaacatctattaatcattaattcgcttctttaatt
ttttataaactaacattgttaattgaaaagggataacatgttacagaatataaattatatatggatttttttaaaaaggaaat
acttgactggagtatatatttatctcttcattatatagcacgcgtgttttccaatttttccacatcccatataatacaggattata
atctcgttcgaacatacgagaaagtggataaaacaatagttgattttttatctaggttgccaaatttattccatattttagaat
atggggaaaatattctacatatttattctatggatgatgctaatacgaatattataattttttttctagatagagtattaaatatt
aataagaacgggtcatttatacacaatctcaggttatcatcatccattaatataaaagaatatgtatatcaattagttaata
atgatcatccagataataggataagactaatgcttgaaaatggacgtagaacaagacattttttgtcctatatatcagat
acagttaatatctatatatgtattttaataaatcatggattttatatagatgccgaagacagttacggttgtacattattacata
gatgtatatatcactataagaaatcagaatcagaatcatacaatgaattaattaagatattgttaaataatggatcagat
gtagataaaaaagatacgtacggaaacacaccttttatcctattatgtaaacacgatatcaacaacgtggaattgtttg
agatatgtttagagaatgctaatatagactctgtagactttaatagatatacacctcttcattatgtctcatgtcgtaataaat
atgattttgtaaagttattaatttctaaaggagcaaatgttaatgcgcgtaatagattcggaactactccattttattgtggaa
ttatacacggtatctcgcttataaaactatatttggaatcagacacagagttagaaatagataatgaacatatagttcgtc
atttaataatttttgatgctgttgaatctttagattatctattatccagaggagttattgatattaactatcgtactatatacaacg
aaacatctatttacgacgctgtcagttataatgcgtataatacgttggtctatctattaaacagaaatggtgattttgagac
gattactactagtggatgtacatgtatttcggaagcagtcgcaaacaacaacaaaataataatggaagtactattgtct
aaacgaccatctttgaaaattatgatacagtctatgatagcaattactaaacataaacagcataatgcagatttattgaa
aatgtgtataaaatatactgcgtgtatgaccgattatgatactcttatagatgtacagtcgctacagcaatataaatggtat
attttaaaatgtttcgatgaaatagatatcatgaagagatgttatataaaaaataaaactgtattccaattagttttttgtatc
aaagacattaatactttaatgagatacggtaaacatccttctttcgtgaaatgcactagtctcgacgtatacggaagtcgt
gtacgtaatatcatagcatctattagatatcgtcagagattaattagtctattatccaagaagctggatcctggagataaa
tggtcgtgttttcctaacgaaataaaatataaaatattggaaaactttaacgataacgaactatccacatatctaaaaat
cttataaacactattaaaatataaaatctaagtaggataaaatcacactacatcattgtttccttttagtgctcgacagtgta
tactatttttaacactcataaataaaaatgaaaacgatttccgttgttacgttgttatgcgtactacctgctgttgtttattcaac
atgtactgtacccactatgaataacgctaaattaacgtctaccgaaacatcgtttaatgataaacagaaagttacgttta
catgtgatcagggatatcattcttcggatccaaatgctgtctgcgaaacagataaatggaaatacgaaaatccatgca
aaaaaatgtgcacagtttctgattacatctctgaattatataataaaccgctatacgaagtgaattccaccatgacacta
agttgcaacggcgaaacaaaatattttcgttgcgaagaaaaaaatggaaatacttcttggaatgatactgttacgtgtc
ctaatgcggaatgtcaacctcttcaattagaacacggatcgtgtcaaccagttaaagaaaaatactcatttggggaata
tatgactatcaactgtgatgttggatatgaggttattggtgcttcgtacataagttgtacagctaattcttggaatgttattcca
tcatgtcaacaaaaatgtgatatgccgtctctatctaatggattaatttccggatctacattttctatcggtggcgttatacat
cttagttgtaaaagtggttttacactaacggggtctccatcatccacatgtatcgacggtaaatggaatcccgtactccca
atatgtgtacgaactaacgaagaatttgatccagtggatgatggtcccgacgatgagacagatttgagcaaactctcg
aaagacgttgtacaatatgaacaagaaatagaatcgttagaagcaacttatcatataatcatagtggcgttaacaatta
tgggcgtcatatttttaatctccgttatagtattagtttgttcctgtgacaaaaataatgaccaatataagttccataaattgct
accgtaaatataaatccgttaaaataatgaataattaataacgaacaagtatcaaaagattaaagaattatagctaga
atcaattgagatgtcttcttcagtggatgttgatatctacgatgccgttagagcatttttactcaggcactattataacaaga
gatttattgtgtatggaagaagtaacgccatattacataatatatacaggctatttacaagatgcgccgttataccgttcg
atgatatagtacgtactatgccaaatgaatcacgtgttaaacaatgggtgatggatacacttaatggtataatgatgaat
gaacgcgatgtttctgtaagcgttggcaccggaatactattcatggaaatgtttttcgattacaataaaaatagtatcaac
aatcaactaatgtatgatataattaatagcgtatctataattctagctaatgagagatatagaagcgcttttaacgacgat
ggtatatacatccgtagaaatatgattaacaagttgtacggatacgcatctctaactactattggcacgatcgctggagg
tgtttgttattatctgttgatgcatctagttagtttgtataaataattatttcaatatactagttaaaattttaagattttaaatgtata
aaaaactaataacgtttttatttgtaataggtgcattagcatcctattcgaataatgagtacactccgtttaataaactgagt
gtaaaactctatatagatggagtagataatatagaaaattcatatactgatgataataatgaattggtgttaaattttaaag
agtacacaatttctattattacagagtcatgcgacgtcggatttgattccatagatatagatgttataaacgactataaaat
tattgatatgtataccattgactcgtctactattcaacgcagaggtcacacgtgtagaatatctaccaaattatcatgccat
tatgataagtacccttatattcacaaatatgatggtgatgagcgacaatattctattactgcagagggaaaatgctataa
aggaataaaatatgaaataagtatgatcaacgatgatactctattgagaaaacatactcttaaaattggatctacttatat
atttgatcgtcatggacatagtaatacatattattcaaaatatgatttttaaaaatttaaaatatattatcacttcagtgacagt
agtcaaataacaaacaacaccatgagatatattataattctcgcagttttgttcattaatagtatacacgctaaaataact
agttataagtttgaatccgtcaattttgattccaaaattgaatggactggggatggtctatacaatatatcccttaaaaatta
tggcatcaagacgtggcaaacaatgtatacaaatgtaccagaaggaacatacgacatatccgcatttccaaagaat
gatttcgtatctttctgggttaaatttgaacaaggcgattataaagtggaagagtattgtacgggactatgcgtcgaagta
aaaattggaccaccgactgtaacattgactgaatacgacgaccatatcaatttgtacatcgagcatccgtatgctacta
gaggtagcaaaaagattcctatttacaaacgcggtgacatgtgtgatatctacttgttgtatacggctaacttcacattcg
gagattctaaagaaccagtaccatatgatatcgatgactacgattgcacgtctacaggttgcagcatagactttgtcac
aacagaaaaagtgtgcgtgacagcacagggagccacagaagggtttctcgaaaaaattactccatggagttcgaa
agtatgtctgacacctaaaaagagtgtatatacatgcgcaattagatccaaagaagatgttcccaatttcaaggacaa
aatggccagagttatcaagagaaaatttaataaacagtctcaatcttatttaactaaatttctcggtagcacatcaaatg
atgttaccacttttcttagcatgcttaacttgactaaatattcataactaatttttattaatgatacaaaaacgaaataaaact
gcatattatacactggttaacgcccttataggctctaaccattttcaagatgaggtccctgattatagtccttctgttcccctc
tatcatctactccatgtctattagacaatgtgagaagactgaagaggaaacatggggattgaaaatagggttgtgtata
attgccaaagatttctatcccgaaagaactgattgcagtgttcatctcccaactgcaagtgaaggattgataactgaag
gcaatggattcagggatatacgaaacaccgataaattataaaaaaagcaatgtgtccgctgtttccgttaataatacta
ttttcgtaactggcggattattcataaataactctaatagcacgatcgtggttaacaatatggaaaaacttgacatttataa
agacaaacaatggtcgattatagaaatgcctatggctagggtatatcacggcatcgactcgacatttggaatgttatatt
ttgccggaggtctatccgttaccgaacaatatggtaatttagagaaaaacaacgagatatcttgttacaatcctagaac
gaataagtggtttgatatttcatatactatttataagatatccatatcatcattgtgtaaactaaataacgtcttctatgtattta
gtaaggacattggatatgtggaaaagtatgatggtgcatggaagttagtacatgatcgtctccccgctataaaggcatt
atcaacttctccttattgattgaaaatgaaaatataaatagtttttatgtatagcagtattaccctatagttttattgcttactact
aacatggatacagatgttacaaatgtagaagatatcataaatgaaatagatagagagaaagaagaaatactaaaa
aatgtagaaattgaaaataataaaaacattaacaagaatcatccaagtggatatattagagaagcactcgttattaat
accagtagtaatagtgattccattgataaagaagttatagaatgtatctgtcacgatgtaggaatatagatcatatctact
aatttttataatcgatacaaaacataaaaaacaactcgttattacatagcaggcatggaatccttcaagtattgttttgata
acgatggcaagaaatggattatcggaaatactttatattctggtaattcaatactatataaggtcagaaaaaatttcacta
gttcgttctacaattacgtaatgaagatagatcacaaatcacacaagccattgttgtctgaaatacgattctatatatctgt
attggatcctttgactatcgacaactggacacgggaacgtggtataaagtatttggctattccagatctgtatggaattgg
agaaaccgatgattatatgttcttcgttataaagaatttgggaagagtattcgccccaaaggatactgaatcagtcttcg
aagcatgcgtcactatgataaacacgttagagtttatacactctcaaggatttacccatggaaaaatagaaccgagga
atatactgattagaaataaacgtctttcactaattgactattctagaactaacaaactatacaagagtggaaactcacat
atagattacaacgaggacatgataacttcaggaaatatcaattatatgtgtgtagacaatcatcttggagcaacagtttc
aagacgaggagatttagaaatgttgggatattgcatgatagaatggttcggtggcaaacttccatggaaaaacgaaa
gtagtataaaagtaataaaacaaaaaaaagaatataaaaaatttatagctactttctttgaggactgttttcctgaagga
aatgaacctctggaattagttagatatatagaattagtatacacgttagattattctcaaactcctaattatgacagactac
gtaaactgtttatacaagattgaaattatattcttttttttatagagtgtggtagtgttacggatatttaatattagactatctctat
cgcgctacacgaccaatatcgattactatggatatcttcagggaaatcgcatcttctatgaaaggagagaatgtattcat
ttctccagcgtcaatctcgtcagtattgacaatactgtattatggagctaatggatccactgctgaacagctatcaaaata
tgtagaaaaggaggagaacatggataaggttagcgctcaaaatatctcattcaaatccataaataaagtatatgggc
gatattctgccgtgtttaaagattcctttttgagaaaaattggcgataagtttcaaactgttgacttcactgattgtcgcactat
agatgcaatcaacaagtgtgtagatatctttactgaggggaaaatcaatccactattggatgaaccattgtctcctgata
cctgtctcctagcaattagtgccgtatactttaaagcaaaatggttgacgccattcgaaaaggaatttaccagtgattatc
ccttttacgtatctccgacggaaatggtagatgtaagtatgatgtctatgtacggcaaggcatttaatcacgcatctgtaa
aggaatcattcggcaacttttcaatcatagaactgccatatgttggagatactagtatgatggtcattcttccagacaaga
ttgatggattagaatccatagaacaaaatctaacagatacaaattttaagaaatggtgtaactctctggaagctacgttt
atcgatgttcacattcccaagtttaaggtaacaggctcgtataatctggtggatactctagtaaagtcaggactgacaga
ggtgttcggttcaactggagattatagcaatatgtgtaattcagatgtgagtgtcgacgctatgatccacaaaacgtatat
agatgtcaatgaagagtatacagaagcagctgcagcaacttgtgcactggtgtcagactgtgcatcaacaattacaa
atgagttctgtgtagatcatccgttcatctatgtgattaggcatgttgatggaaaaattcttttcgttggtagatattgctctcc
gacaactaattgttaaccattttttttaaaaaatagaaaaaacatgtggtattagtgcaggtcgttattcttccaattgcaatt
ggtaagatgacggccaactttagtacccacgtcttttcaccacagcactgtggatgtgacagactgaccagtattgatg
acgtcagacaatgtttgactgaatatatttattggtcgtcctatgcataccgcaacaggcaatgcgctggacaattgtatt
ccacactcctctcttttagagatgatgcggaattagtgttcatcgacattcgcgagctggtaaaaaatatgccgtgggat
gatgtcaaagattgtgcagaaatcatccgttgttatataccggatgagcaaaaaaccatcagagagatttcggccatc
atcggactttgtgcatatgctgctacttactggggaggtgaagaccatcccactagtaacagtctgaacgcattgtttgtg
atgcttgagatgctcaattacgtggattataacatcatattccggcgtatgaattgatgagttgtacatcttgacattttcttctt
tcttctcttctccctttcccagaaacaaactttttttacccactataaaataaaatgagtatactacctgttatatttctttctatat
ttttttattcttcattcgttcagacttttaacgcgcctgaatgtatcgacaaagggcaatattttgcatcattcatggagttaga
aaacgagccagtaatcttaccatgtcctcaaataaatacgctatcatccggatataatatattagatattttatgggaaaa
acgaggagcggataatgatagaattataccgatagataatggtagcaatatgctaattctgaacccgacacaatcag
actctggtatttatatatgcattaccacgaacgaaacctactgtgacatgatgtcgttaaatttgacaatcgtgtctgtctca
gaatcaaatatagatcttatctcgtatccacaaatagtaaatgagagatctactggcgaaatggtatgtcccaatattaa
tgcatttattgctagtaacgtaaacgcagatattatatggagcgggcatcgacgccttagaaataagagacttaaaca
acggacacctggaattattaccatagaagatgttagaaaaaatgatgctggttattatacatgtgttttagaatatatatac
ggtggcaaaacatataacgtaaccagaattgtaaaattagaggtacgggataaaataataccttctactatgcaatta
ccagatggcattgtaacttcaataggtagtaatttgactattgcatgcagagtatcgttgagacctcccacaacggatgc
agacgtcttttggataagtaatggtatgtattacgaagaagatgatggggacggaaacggtagaataagtgtagcaa
ataaaatctatatgaccgataagagacgtgttattacatcccggttaaacattaatcctgtcaaggaagaagatgctac
aacgtttacgtgtatggcgtttactattcctagcatcagtaaaacagttactgttagtataacgtgaatgtatgttgttacattt
ccatgtcaattgagtttataagaatttttatacattatcttccaacaagcaattgacgaacgtattgctatgattaactccca
cgatactatgcatattattaatcattaacttgcagactatacctagagctattttgacatactcgtgttcttgtgtaattgcagt
atctatattattaaagtacgtaaatctagctatagttttattatttaattttagataatataccgtctccttatttttaaaaattgcc
acatcctttattaaatcatgaatgggaatttctatgtcatagttaatatattgtgaacaacaagagcagatatctataggaa
agggtggaatgcgatacattgatctatgtagttttaaaacacacgcaaactttgaagaatttatataaatcattccatcga
tacatccttctatgttgagatgtatatatccaggaattcgtttattaatatcgggaaatgtataaactaaaacattgcccgg
aagcggagcttctaccggagttatatcagtttttaacttacaaaatgtaaccaatacctttgcatgacttgttttgttcggca
acgttagtttaaacttgacgaatggattaattacaatagcatgatccgcgcatctattaagtttttttactttaacgcccttgta
tgtttttacagagactttatctaaatttctagtgcttgtatgtgttataaatataacgggatatagaactgaatcacctacctta
gatacccaattacattttatcagatccagataataaacaaattttgtcgccctaactaattctatattgttatatattttacaatt
ggttatgatatcatgtaataacttggaatctaacgcacatcgtcgtacgtttatacaattgtgatttagtgtagtatatctaca
catgtatttttccgcgctatagtattctggactagtgataaaactatcgttatatctgtcttcaatgaactcatcgagatattgc
tctctgtcatattcatacacctgcataaactttctagacatcttacaatccgtgttattttaggatcatatttacatatttacggg
tatatcaaagatgttagattagttaatgggaatcgtctataataatgaatattaaacaattatatgaggacttttaccacaa
agcatcataaaaatgagtcgtcgtctgatttatgttttaaatatcaaccgcgaatcaactcataaaatacaagagaatg
aaatatatacatattttagtcattgcaatatagaccatacttctacagaacttgattttgtagttaaaaactatgatctaaac
agacgacaacctgtaactgggtatactgcactacactgctatttgtataataattactttacaaacgatgtactgaagata
ttattaaatcatggagtggatgtaacgatgaaaaccagtagcggacgtatgcctgtttatatattgcttactagatgttgca
atatttcacatgatgtagtgatagatatgatagacaaagataaaaaccacttattacatagagactattccaacctatta
ctagagtatataaaatctcgttacatgttattaaaggaagaggatatcgatgagaacatagtatccactttattagataag
ggaatcgatcctaactttaaacaagacggatatacagcgttacattattattatttgtgtctcgcacacgtttataaaccag
gtgagtgtagaaaaccgataacgataaaaaaggccaagcgaattatttctttgtttatacaacatggagctaatctaaa
cgcgttagataattgtggtaatacaccattccatttgtatcttagtattgaaatgtgtaataatattcatatgactaaaatgct
gttgacttttaatccgaatttcgaaatatgtaataatcatggattaacgcctatactatgttatataacttccgactacataca
acacgatattcttgttatgttaatacatcactatgaaacaaatgttggagaaatgccgatagatgagcgtcgtataatcgt
attcgagtttatcaaaacatattctacacgtcctgcagattcgataacttatttgatgaataggtttaaaaatatagatattta
tacccgctatgaaggaaagacattattacacgtagcatgtgaatataataatacacacgtaatagattatcttatacgta
tcaacggagatataaatgcgttaaccgacaataacaaacacgctacacaactcattatagataacaaagaaaattc
cccatataccattaattgtttactgtatatacttagatatattgtagataagaatgtgataagatcgttggtggatcaacttcc
atctctacctatcttcgatataaaatcatttgagaaattcatatcctactgtatacttttagatgacacattttacaatagaca
cgttaggaatcgcgattctaaaacgtatcgatacgcattttcaaaatacatgtcgtttgataaatacgatggtataataac
taaatgtcataaagaaacaatattgctcaaactatccactgttctagacactacactatatgcagttttaagatgccataa
ttcgaaaaagttaagaagatacctcaccgagttaaaaaaatataataacgataagtcctttaaaatatattctaatattat
gaatgagagataccttaatgtatattataaagatatgtacgtgtcaaaggtatatgataaactatttcctgttttcacagata
aaaattgtctactaacattactaccttcagaaattatatacgaaatattatacatgctgacaattaacgatctttataatata
tcgtatccacctaccaaagtatagttgtatttttctcatgcgatgtgtgtaaaaaaactgatattatataaatattttagtgcc
gtataataaagatgacgatgaaaatgatggtacatatatatttcgtatcattattgttattgctattccacagttacgccata
gacatcgaaaatgaaatcacagaattcttcaataaaatgagagatactctaccagctaaagactctaaatggttgaat
ccagcatgtatgttcggaggcacaatgaatgatatagccgctctaggagagccattcagcgcaaagtgtcctcctattg
aagacagtcttttatcgcacagatataaagactatgtggttaaatgggaaaggctagaaaaaaatagacggcgaca
ggtttctaataaacgtgttaaacatggtgatttatggatagccaactatacatctaaattcagtaaccgtaggtatttgtgc
accgtaactacaaagaatggtgactgtgttcagggtatagttagatctcatattagaaaacctccttcatgcattccaaa
aacatatgaactaggtactcatgataagtatggcatagacttatactgtggaattctttacgcaaaacattataataatat
aacttggtataaagataataaggaaattaatatcgacgacattaagtattcacaaacgggaaaggaattaattattcat
aatccagagttagaagatagcggaagatacgactgttacgttcattacgacgacgttagaatcaagaatgatatcgta
gtatcaagatgtaaaatacttacggttataccgtcacaagaccacaggtttaaactaatactagatccaaaaatcaac
gtaacgataggagaacctgccaatataacatgcactgctgtgtcaacgtcattattgattgacgatgtactgattgaatg
ggaaaatccatccggatggcttataggattcgattttgatgtatactctgttttaactagtagaggcggtattaccgaggc
gaccttgtactttgaaaatgttactgaagaatatataggtaatacatataaatgtcgtggacacaactattattttgaaaaa
acccttacaactacagtagtattggagtaaatatacaatgcatttttatatacattactgaataattattattattatttatatcg
tatttgtgctataacgcgactatctaggtatttgtatctcaccgatagagaacatataaatgtagactctattaaacagttgt
gtaaaatatcagatcctaatgcatgttatagatgtggatgtacggctttacatgagtacttttataattatagatcagtcaac
ggaaaatacaagtatagatacaacggttactatcaatattatttatctagcgattatgaaaattataatgaatattattatg
atgattatgatagaactggtatgaacagtgagagtgataatatatcaatcaaaacagaatatgaattctatgatgaaac
acaagatcaaagtacacaactagtaggttacgacattaaactcaaaaccaatgaggatgattttatggctatgataga
tcagtgggtgtccatgattatatagatgaatcaattaataaagtagtatatggaagagagtctcacgtaagatggcggg
atatatggcaagaacataatgatggcgtatacagtataggaaaggagtgcatagataatatatacgaagacaaccat
accgtagacgaattctacaagatagacagcgtatcagatgtagatgacgcggaacacatatctccgataactaatga
tgtatctacacaaacatgggaaaagaaatcagagttagatagatacatggaaatgtatcctcgtcatagatatagtaa
gcattctgtctttaagggattttctgacaaagttagaaaaaatgatttagacatgaatgtggtaaaagaattactttctaac
ggtgcatctctaacaattaaggatagcagtaataaggatccaataaccgtttattttcgaagaacgataatgaatttaga
aatgattgatgaacgaaagtatatagtacactcctatctaaaaaattataaaaatttcgattatccatttttcaggaagtta
gttttgactaataaacattgtctcaacaattattataatataagcgacagcaaatatggaacaccgctacatatattggcg
tctaataaaaaattaataactcctaattacatgaagttattagtgtataacggaaatgatataaacgcacgaggtgaag
atacacaaatgcgaactccattacacaaatatttgtgtaaatttgtatatcataatattgaatatggtatccgatactataat
gaaaagattatagacgcatttatagagttaggagccgatctaactattccaaatgacgatggaatgataccagtagttt
actgtatacactcaaatgccgaatatggttataacaatattactaacataaagataatacgtaaactacttaatcttagta
gacgtgcgtcacataatctatttagagatcgagtcatgcacgattatataagtaatacatatattgatcttgagtgtttagat
attattagatcgttggatggattcgatatcaatggttactttgaaggacgtacaccacttcattgcgctatacaacataactt
cactcagattgctaagtacttattagatcgaggagctgatatagtcgtacccaacacattgattatacatcagtacatac
agtaaatagcatagatatggaggaggatacaaatatttcaaataaagttataaggtacaacactgtcaataatatatg
ggaaacattacctaacttctggactggaactataaatccaggcgtggtctcgcataaagatgatatatatgttgtatgcg
acatcaaagatgaaaaaaatgttaaaacttgtatatttagatataacacgaatacgtataacggatgggaattggtcac
gacgacagaaagcagattatcagctctgcatactattctttataacaataccataatgatgttacattgttatgaatcgtat
atgttacaagatacatttaatgtgtacactcgcgaatggaatcatatgtgtcatcaacattcgaatagttatatcatgtaca
atatactacccatctactaaatataatagaataaaataaatgagtatgatcattttagataacgattgattttatcattaccg
cttcattcttatattctttgcttacggaacctatatttagaaacatctactaacgattttttatgcttgcattattaatggtatgtaat
atgattgattgtgtacgcaataccaatttgttaagtatgaatacggggtacaaacataaactgaagtttaacattatttattt
atgatatatatcgttattgtttggtctataccatggatatctttaaagaactaatcttaaaacacacggatgaaaatgttttga
tttctccagtttctattttatctactttatctattctaaatcatggagcagctggttctacagctgaacaactatcaaaatatata
gagaatatgaatgagaatacacccgatgacaataatgacatggacgtagatattccgtattgtgcgacactagctacc
gcaaataaaatatacggtagcgatagtatcgagttccacgcctccttcctacaaaaaataaaagacgattttcaaact
gtaaactttaataatgctaaccaaacaaaggaactaatcaacgaatgggttaagacaatgacaaatggtaaaattaa
ttccttattgactagtccgctatccattaatactcgtatgacagttgttagcgccgtccattttaaagcaatgtggaaatatcc
attttctaaacatcttacatatacagacaagttttatatttctaagaatatagttaccagtgttgatatgatggtgagcactga
gaataacttgcaatatgtacatattaatgaattattcggaggattctctattatcgatattccatacgagggaaactctagt
atggtaattatactaccggacgacatagaaggtatatataacatagaaaaaaatataacagatgaaaaatttaaaaa
atggtgtggtatgttatctactaaaagtatagacttgtatatgccaaagtttaaagtggaaatgacagaaccgtataatct
ggtaccgattttagaaaatttaggacttactaatatattcggatattatgcagattttagcaagatgtgtaatgaaactatca
ctgtagaaaaatttctacatacgacgtttatagatgttaatgaggagtatacagaagcatcggccgttacaggagtattt
atgactaacttttcgatggtatatcgtacgaaggtctacataaaccatccattcatgtacatgattaaagacaacacagg
acgtatactttttatagggaaatactgctatccgcaataaatataaacaaatagacttttatcacgtttatctatgtctaaat
attacaaatagtaatagtataaactaaagctgataatacttaaaaaaataataatatcatttacaattaatagtataaact
aaaaattaaacaaatcgttattataagtaatatcaaaatgatgatatacggattaatagcgtgtcttatattcgtgacttcat
ccatcgctagtccactttatattcccgttattccacccatttcggaagataaatcgttcaatagtgtagaggtattagtttcctt
gtttagagatgaccaaaaagactatacggtaacttctcagttcaataactacactatcgataccaaagactggactatc
ggcgtactatccacacctgatggtttggatataccattgactaatataacttattggtcacggtttactataggtcgtgcatt
gttcaaatcagagtctgaggatattttccaaaagaaaatgagtattctaggtgtttctatagaatgtaagaagtcgtcgac
attacttacttttttgaccgtgcgtaaaatgactcgagtatttaataaatttccagatatggcttattatcgaggagactgttta
aaagccgtttatgtaacaatgacttataaaaatactaaaactggagagactgattacacgtacctctctaatggggggt
tgcctgcatactatcgtaatggggtcgatggttgattattgattagtatattccttattctttttattcacacaaaaagaacattt
ttataaacatgaaaccactgtctaaatgtaattatgatcttgatttatagatgaagatcagcctttagaggattttaaccagt
atgtttaatatgaaaaaaataaacataacatattttgagattaagcgctattgtgcaagattatattagaatcaaattaatc
tttcatacgagaaaaataacgacatacgtcgtcaacaaattaaactttttatttattagttaactagcttatagaacttgctc
attgttatgtttctaaaacgggtacgacatataggacaattatccgacgcaccggtttctcttcgtgttttatgccatatattg
atgcatgttatgcaaaatatatgagtacacgaatccaataaaccaaagtatctatcgttttgagtaaacaacttcatagc
aaattccacattctttttctttacttactctatacacgtcctcgtatttatccagtattttgatgatatccaactcagaaatggttgt
tgtattattgggtgtattgggagtataggtattattagctatgtaccaatttaccaaccctcttaatattgattgaacaatcac
atcggttatccaatcaataaccacattaataactaaattgtagtgtatatatagaccatatatgtttctatttttttgacagtta
cgtatagtttcagtaagttttgattgttgtattcctgtatctctagataagttagtcatatagtcccttccggcgatacgttttttcc
aagcccgaaattgattagccaaatgtgtatttatttttgtgatattgatataatatgtaatgttattaatatttcggataatgcat
actgttagtcttatatcatttggttcatctatgtattgtaatattgttacatgatctatagatgatgtattgattttggcaggatcga
attccatatccgcgactaaacagtgaaaaaaatgtaaatactttttaaattttaaattagtaaaacttttttttattttttatgatt
ccaaaaatactgaatacaaagtcctaaattataaatatggagatcatactaccacaacttattattatgcatactcagcc
ggtgtaatagatagatatatataattctattacaccggcagacaattaccgatcggtatttgtcgttaccaacataccgtat
aatatgtaatatacaattccataacccattgacagttgttatacatcaaaattgcaattcttttgattacgatgttataagaat
gtagttaattgatgtatgatgttaatgtgtcctctttcctcttataacatcgtaatcaaaaacttttttataatatatacctaataa
tgtgtcttaatagttctcgtgattcgtcaaacaatcattcttataaaatataataaagcaacgtaaaacacataaaaataa
gcgtaactaataagacaatggatatttacgacgataaaggtctacagactattaaactgtttaataatgaatttgattgtat
aaggaatgacatcagagaattatttaaacatgtaactgattccgatagtatacaacttccgatggaagacaattctgat
attatagaaaatatcagaaaaatactatatagacgattaaaaaatgtagaatgtgttgacatcgatagtacaataacttt
tatgaaatacgatccaaatgatgataataagcgtacgtgttctaattgggtacccttaactaataactatatggaatattgt
ctagtaatatatttggaaacaccgatatgtggaggcaaaataaaattataccaccctacaggaaatataaagtcggat
aaggatattatgtttgcaaagactctagactttaaatcaaagaaagtgttaactggacgtaaaacaattgccgttctaga
catatccgtttcatataatagatcaatgactactattcactacaacgacgacgttgatatagatatacatactgataaaaa
tggaaaagagttatgttattgttatataacaatagatgatcattacttggttgatgtggaaactataggagttatagtcaata
gatctggaaaatgtctgttagtaaataaccatctaggtataggtatcgttaaagataaacgtataagcgatagttttgga
gatgtatgtatggatacaatatttgacttttctgaagcacgagagttattttcattaactaatgatgataacaggaatatagc
atgggacactgataaactagacgatgatacagatatatggactcccgtcacagaagatgattacaaatttctttctaga
ctagtattgtatgcaaaatctcaatcggatactgtattcgactattatgttcttactggtgatacggaaccacccactgtatt
cattttcaaggtaactagattttactttaatatgccgaaataaaaaatttttgtataatatctagaggtagaggtattgtttag
ataaatacaaataacatagatacatcgcatacttagcatttttataaatatacataagacatacactttatacattttttttgt
aaaaatactcataaaaaaatttataaaaattatggcacaaccatatcttgtataggtagtttagttcgtcgagtgaaccta
taaacagataatagacaacacgtaataataataatgcctactaatacaagcataataccgggagatgggatatatga
cgttgtagtgtttgggttttctgaacgttgatagtctactaatactacatgctgacatctaatgcctgtataaccatgagagc
atctacaatacataccgtcaatatctctagcgtggatacagtcaccgtgtaaacaatatccatctccctctggaccgcat
aatctgatagctggaatatctgttgtagcgtttgtaatttctggcgatgtcgtttcgatagcgttaccactatcggcgaatgat
ctgattatcatagcagcgaacaacaacatcagatatttcatcgacatttttgatggattttgtgtttatgctgtttctcagtgtgt
gtttatgacaagattgggaattttatattattaattcagtaatataaactaataatatattgttaattgtgtaaataatataaaa
ataacaatacaatattgaatgtgttgctgttaaaaatgatcataaacacggagtttattttatatgtctcgcataaacattac
taaaaaaatatattgttctgtttttctttcacatctttaattatgaaaaagtaaatcattatgagatggacgagattgtacgcat
cgttcgcgacagtatgtggtacatacctaacgtatttatggacgacggtaagaatgaaggtcacgtttctgtcaacaatg
tctgtcatatgtatttcacgttctttgatgtggatacatcgtctcatctgtttaagctagttattaaacactgcgatctgaataaa
cgaggtaactctccattacattgctatacgatgaatacacgatttaatccatctgtattaaagatattgttacaccacggc
atgcgtaactttgatagcaaggatgaaaaaggacaccactatcaatcgataacaagatctttgatatactaacggaca
ccattgatgactttagtaaatcatccgatctattgctgtgttatcttagatataaattcaatgggagcttaaactattacgttct
gtacaaaggatccgacgaggatgaactcacttctcttcattactactgtaaacacatatccacgttctacaaaagcaatt
attacaagttaagtcacactaagatgcgagccgagaagcgattcatctacgcgataatagattatggagcaaacatta
acgcggttacacacttaccttcaacagtataccaaacatagtcctcgtgtggtgtatgctcttttatctcgaggagccgat
acgaggatacgtaataatcttgattgtacacccatcatggaacgattgtgcaacaggtcatattctcataatgttactcaa
ttggcacgaacaaaaggaagaaggacaacatctactttatctattcataaaacataatcaaggatacactctcaatat
actacggtatctactagataggttcgacattcagaaagacgaatactataataccgcctttcaaaattgtaacaacaat
gttgcctcatacatcggatacgacatcaaccttccgactaaagacggtattcgacttggtgtttgaaaacagaaacatc
atatacaaggcggatgttgtgaatgacatcatccaccacagactgaaagtatctctacctatgattaaatcgttgttctac
aagatgtctctccctacgacgattactacgtaaagaagataatagcctactgcctattaagggacgagtcattcgcgg
aactacatagtaaattctgtttaaacgaggactataaaagtgtatttatgaaaaatatatcattcgataagatagattccat
catcgtgacataagtcgccttaaagagattcgaatctccgacaccgacctgtatacggtatcacagctatcttaaagcc
atacattcagacagtcacatttcatttcccatgtacgacgatctcatagaacagtgccatctatcgatggagcgtaaaag
taaactcgtcgacaaagcactcaataaattagagtctaccatcggtcaatctagactatcgtatttgcctccggaaattat
gcgcaatatcatctaaacagtatgttgtacgaaaagaaccattacaaatattatccatgatagaaagaaaatatctata
tgattggagaagtaggaaacaggaacaagacgacgattactacattattaaatcatgaagtccgtattatactcgtata
tattgtttctctcatgtataataataaacggaagagatatagcaccgcatgcaccatccgatggaaagtgtaaagacaa
cgaatacaaacgccataatttgtgtccgggaacatacgcttccagattatgcgatagcaagactaacacacaatgtac
gccgtgtggttcgggtaccttcacatctcgcaataatcatttacccgcttgtctaagttgtaacggaagacgcgaccgtgt
aacacgactcacaatagaatctgtgaatgctctcccggatattattgtcttctcaaaggatcatccggatgcaaggcatg
tgtttcccaaacaaaatgtggaataggatacggagtatccggagacgtcatctgttctccgtgtggtctcggaacatatt
ctcacaccgtctcttccgcagataaatgcgaacccgtacccagtaatacctttaactatatcgatgtggaaattaatctgt
atccagttaacgaccactaccggtctcagcgaatccatctcaacgtcggaactaactattactatgaatcataaagact
gtaatcccgtatttcgtgaggaatacttctccgtccttaataaggtagcaacttcaggtttctttacaggagaaaggtgtgc
actctgaatttcgagattaaatgcaataacaaaaatttttcctccaaacagttaacgaaagcaaagaatgatgacggt
atcatgccgcattcggagactgtctatctagcgtcgacatctatatactatatagtaataccaatactcaagactacgaa
actgatacaatctcttatcatgtgggtaatgttctcgatgtcgatagccatatgcccggtagttgcgatatacataaactga
tcactaattccaaacccacccgctttttatagtaagtttttcacccataaataataaatacaataattaatttctcgtaaaag
tagaaaatatattctaatttattgcacggtaaggaagtagaatcataaagaacagtactcaatcaatagcaatcatgaa
acaatatatcgtcctggcatgcatgtgcctgccagtcttcagcaatcatcctcatcctcctcctcgtgtacggaagaaga
aaacaaacatcatatgggaatcgatgttattatcaaagtcacaaagcaagaccaaacaccgaccaatgataagattt
gccaatccgtaacggaaattacagagtccgagtcagatccagatcccgaggtggaatcagaagatgattccacatc
agtcgaggatgtagatcctcctaccacttattactccatcatcggtggaggtctgagaatgaactttggattcaccaaat
gtcctcagattaaatccatctcagaatccgctgatggaaacacagtgaatgctagattgtccagcgtgtccccaggac
aaggtaaggactctcccgcgatcactcatgaagaagctctggctatgatcaaagactgtgaggtgtctatcgacatca
gatgtagcgaagaagagaaagacagcgacatcaagacccatccagtactcgggtctaacatctctcataagaaag
tgagttacgaagatatcatcggttcaacgatcgtcgatacaaaatgtgtcaagaatctagagtttagcgttcgtatcgga
gacatgtgcaaggaatcatctgaacttgaggtcaaggatggatttaagtatgtcgacggatcggcatctgaaggtgca
accgatgatacttcactcatcgattcaacaaaactcaaagcgtgtgtctgaatcgataactctattcatctgaaattggat
gagtagggttaatcgaacgattcaggcacaccacgaattaaaaaagtgtaccggacactatattccggtttgcaaaa
caaaaatgttcttaactacattcacaaaaagttacctctcgcgacttcttctttttctgtctcaatagtgtgatacgattatgac
actattcctattcctattcctatttcctttcagggtatcacaaaaatattaaacctctttctgatggtctcataaaaaaagtttta
caaaaatatttttattctctttctctctttgatggtctcataaaaaaagttttacaaaaatatttttattctctttctctctttgatggtc
tcataaaaaaagttttacaaaaatatttttattctctttctctctttgatggtctcataaaaaaagttttacaaaaatatttttattc
tctttctctctttgatggtctcataaaaaaagttttacaaaaatatttttattctctttctctctttgatggtctcataaaaaaagtttt
acaaaaatatttttattctctttctctctttgatggtctcataaaaaaagttttacaaaaatatttttattctctttctctctttgatggt
ctcataaaaaaagttttacaaaaatatttttattctctttctctctttgatggtctcataaaaaatattaaacctctttctgatggt
gtcactaaaatatttttattctcattctcattttctctttctctcttcaatggagtcataaaatatttttattctctttctctcttcgatggt
ctcacaaaaatattaaacctctttctgatggtgtcactaaaatatttttattctcattctcattttctctttctctcttcaatggagtc
ataaaatatttttattctctttctctcttcgatggtctcacaaaaatattaaacctctttctgatggagtcgtaaaaaagttttatc
tctttctctcttcgatggtctcacaaaaatattaaacctctttctgatggagtcgtaaaaaagttttatctctttctctcttcgatg
gtctcacaaaaatattaaacctctttctgatggagtcgtaaaaaagttttatctctttctctcttcgatggtctcacaaaaata
ttaaacctctttctgatggagtcgtaaaaaagttttatctctttctctcttcgatggtctcacaaaaatattaaacctctttctga
tggagtcgtaaaaaagttttatctctttctctcttcgatggtctcacaaaaatattaaacctctttctgatggagtcgtaaaaa
agttttatctctttctctcttcgatggtctcacaaaaatattaaacctctttctgatggagtcgtaaaaaagttttatctctttctct
cttcgatggtctcacaaaaatattaaacctctttctgatggagtcgtaaaaaagttttatctctttctctcttcgatggtctcac
aaaaatattaaacctctttctgatggagtcgtaaaaaagttttatctctttctctcttcgatggtctcacaaaaatattaaacc
tctttctgatggagtcgtaaaaaagttttatctctttctctcttcgatggtctcacaaaaatattaaacctctttctgatggagtc
gtaaaaaagttttatctctttctctcttcgatggtctcacaaaaatattaaacctctttctgatggagtcgtaaaaaagttttat
ctctttctctcttcgatggtctcacaaaaatattaaacctctttctgatggagtcgtaaaaaagttttatctctttctctcttcgat
ggtctcacaaaaatattaaacctctttctgatggagtcgtaaaaaagttttatctctttctctcttcgatggtctcacaaaaat
attaaacctctttctgatggagtcgtaaaaaagttttatctctttctctcttcgatggtctcacaaaaatattaaacctctttctg
atggagtcgtaaaaaagttttatctctttctctcttcgatggtctcacaaaaatattaaacctctttctgatggagtcgtaaaa
aagttttatctctttctctcttcgatggtctcacaaaaatattaaacctctttctgatggagtcgtaaaaaagttttatctctttct
ctcttcgatggtctcactaaaatattttttattctctttctgatgcatcaactatttcttaaacaataacgtccaacaacatatac
tcgtcgagcttatcaacatcccctatgcccatctaggttaccagacaattgtatatcataaaataatgtttataatttacacg
ttaaaatcatataataaaacgtagatcgtataatattttttggtatataaatgatctagtaaaatccatgtaggggatactg
ctcacattttttctttggtacaaaatttcacacaagtttttatacagacaaattcttgtccatatattttaaaacattgacttttgta
ctaagaaaaatatctagatcaactatctctttctctttctctcttcgatggtctcacaaaaatattaaacctctttctgatggag
tcgtaaaaaagttttatctctttctctcttcgatggtctcacaaaaatattaaacctctttctgatggagtcgtaaaaaagtttt
atctctttctctcttcgatggtctcacaaaaatattaaacctctttctgatggagtcgtaaaaaagttttatctctttctctcttcg
atggtctcacaaaaatattaaacctctttctgatggagtcgtaaaaaagttttatctctttctctcttcgatggtctcacaaaa
atattaaacctctttctgatggagtcgtaaaaaagttttatctctttctctcttcgatggtctcacaaaaatattaaacctctttc
tgatggagtcgtaaaaaagttttatctctttctccttcgatggtctcacaaaaatattaaacctctttctgatggagtcgtaaa
aaagttttatctctttctctcttcgatggtctcacaaaaatattaaacctctttctgatggagtcgtaaaaaagttttatctctttc
tctcttcgatggtctcacaaaaatattaaacctctttctgatggagtcgtaaaaaagttttatctctttctccttcgatggtctca
caaaaatattaaacctctttctgatggagtcgtaaaaaagttttatctctttctccttcgatggtctcacaaaaatattaaac
ctctttctgatggagtcgtaaaaaagttttatctctttctctcttcgatggtctcacaaaaatattaaacctctttctgatggagt
cgtaaaaaagttttatctctttctctcttcgatggtctcacaaaaatattaaacctctttctgatggtctctataaagcgatcg
atctttcttacactctagagtttcctacagtcatgggtcacacatttttttctagacactaaataaaatatttaaaatataatatt
aatgtactaaaacttatatattattaatttatctaactaaagttagtaaattatatatataattttataattaatttaattttactaat
tttatttagtgtctagaaaaaaa

To evaluate the removal of the bacterial vector sequences following sMVA reconstitution, DNA of sMVA-infected BHK cells upon serial virus passage of sMVA in BHK cells was evaluated by PCR using primers specific for the sopA and cat chloramphenicol resistance gene of the bacterial vector. This PCR analysis showed that residual vector sequences can be detected in sMVA DNA at 7 or 8 dpt/i, although they were undetectable in sMVA DNA following two additional virus passages of sMVA. These results in sum provide evidence that co-transfection of the three synthetic MVA fragments as circular plasmid molecules into FPV-infected BHK cells leads to the formation of sMVA with full-length, vector-free genomes that appear to have similar sequence composition as the published genome sequence of MVA strain Antoine²⁶.

### Example 2: Generation of Recombinant sMVA Expressing a Single Heterologous Gene Sequence

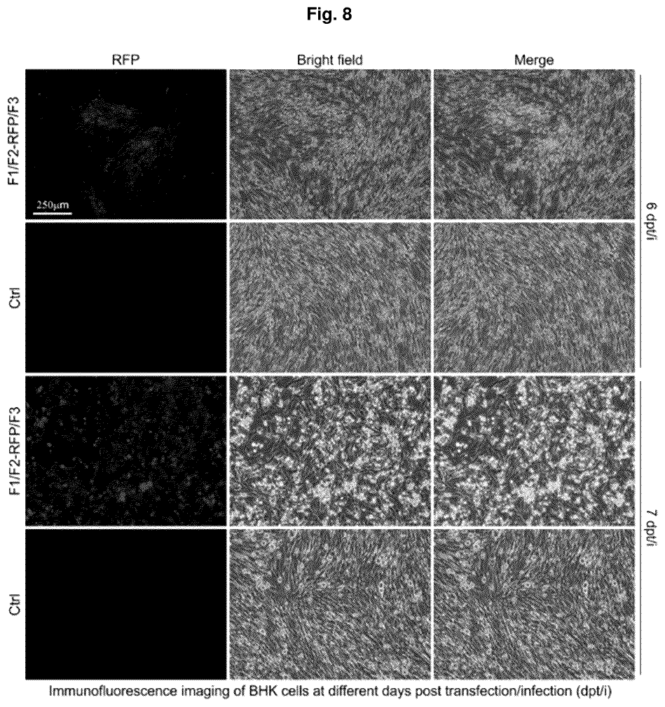

To evaluate the use of the three sMVA fragments to generate rsMVA, the three fragments were transferred into GS1783 E. coli cells⁴⁰, which are used to manipulate large BAC-cloned DNA molecules by En Passant mutagenesis^{39,40}. This highly-efficient method allows the introduction of point mutations or large or small sequence insertions or deletions into BAC cloned DNA molecules by a two-step mutagenesis procedure based on Red recombination without leaving behind any bacterial marker sequences^{39,40}. As an initial test of the sMVA platform to generate rsMVA expressing heterologous gene sequences, the generation of rsMVA expressing a single fluorescence marker was evaluated. As previously described for the insertion of antigen sequences into MVABAC-TK^{18,24}, En passant mutagenesis was used to insert an expression cassette composed of a red fluorescent protein (RFP) marker with upstream mH5 promoter and downstream TTTTTAT vaccinia virus transcription termination signal into the IGR69/70 (also known as G1L/18R) insertion site within sMVA fragment F2 (FIG. 3), resulting in F2-RFP. Sequence integrity of the manipulated sMVA fragments was determined by restriction fragment length polymorphism (RFLP) analysis as well as PCR and Sanger sequencing analysis of the gene insertion sites. The modified sMVA fragment F2 with inserted RFP marker as well as unmodified sMVA fragments F1 and F3 were isolated from E. coli and co-transfected into BHK cells to evaluate the reconstitution of rsMVA in the presence of FPV as a helper virus using the procedure as illustrated in FIG. 5. As shown in FIG. 8, immunofluorescence imaging revealed the formation and progression of red fluorescent viral plaques at 3-6 dpt/i in BHK cell monolayers transfected with the F1/F2-RFP/F3 plasmid combination, while at 7 dpt/i RFP expression was visible in the entire BHK cell monolayer. In contrast, no RFP expression was visible in mock-transfected/infected BHK cell monolayers. These results in sum demonstrate that the sMVA platform can be used to rapidly generate rsMVA expressing a single heterologous gene sequence.

### Example 3: Generation of Recombinant sMVA Expressing Two Heterologous Gene Sequences

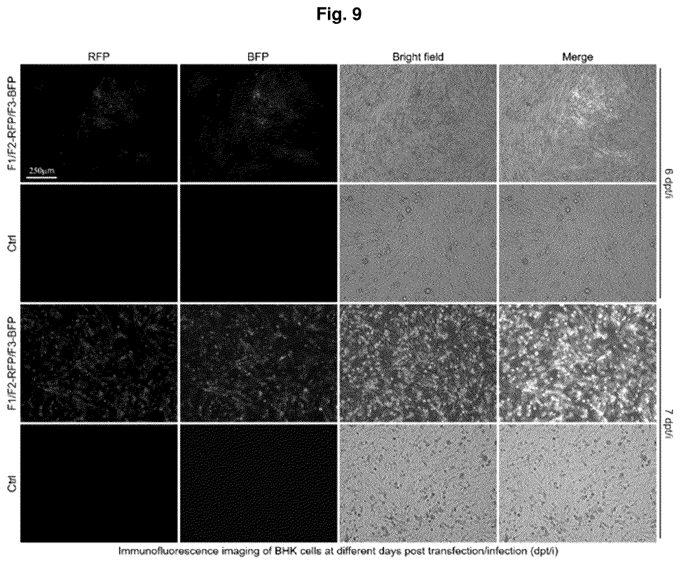

To evaluate the sMVA platform for the generation of rsMVA with multiple heterologous gene sequences inserted into different genome positions, the construction of rsMVA expressing two fluorescence markers was evaluated. Using En passant mutagenesis of GS1783 cells, gene expression cassettes composed of a P11 promoter, an RFP marker or blue fluorescent protein (BFP) marker, and TTTTTAT transcription termination signal were separately inserted into the IGR69/70 of sMVA F2 and the Del3 site of sMVA F3. The modified sMVA F2 with inserted RFP marker (F2-RFP) and the modified sMVA F3 with inserted BFP marker (F3-BFP) were isolated from GS1783 E. coli cells and co-transfected together with unmodified sMVA F1 into BHK cells to evaluate the reconstitution of rsMVA with RFP and BFP marker in the presence of a helper virus using the procedure shown in FIG. 5. As shown in FIG. 9, immunofluorescence imaging demonstrated the formation of double fluorescent (red and blue) viral plaques at 3-6 dpt/i in the BHK cell monolayers co-transfected with the F1/F2-RFP/F3-BFP plasmid combination, and at 7 dpt/i most or all of the BHK cells appeared to show RFP and BFP dual fluorescent protein expression. These results provide evidence that the sMVA platform can be used to rapidly generate rsMVA expressing two heterologous gene sequences inserted into different insertion sites.

### Example 4: Generation of Recombinant sMVA Expressing Three Heterologous Gene Sequences

To further evaluate the sMVA platform for the generation of rsMVA with multiple inserted heterologous gene sequences, the generation of rsMVA expressing three fluorescence markers was investigated. Using En passant mutagenesis in E. coli, a green fluorescent protein (GFP) marker together with upstream P11 promoter and downstream transcription termination signal was inserted into the IGR44/45 of MVA F1. The resulting modified sMVA fragment F1 with inserted GFP marker (F1-GFP) was then tested in different combinations with the fluorescence tagged forms of F2 and F3 (F2-RFP and F3-BFP) as well as unmodified sMVA fragments of F1, F2, and F3 to evaluate the single, double, or triple fluorescent sMVA expression vectors using the procedure shown in FIG. 5. The fragment combinations used for co-transfection included F1-GFP/F2-RFP/F3-BFP, F1-GFP/F2-RFP/F3, F1-GFP/F2/F3-BFP, F1/F2-RFP/F3-BFP, F1-GFP/F2/F3, F1/F2-RFP/F3, and F1/F2/F3-BFP. Co-transfection of the three non-modified sMVA fragments (F1/F2/F3) into FPV-infected BHK cells was evaluated as a negative control. With the exception of the negative control, fluorescent gene expression was observed by immunofluorescence imaging at 3-6 dpt/i in BHK cell monolayers transfected with any of the different fragment combinations. At 7 to 8 dpt/i, fluorescence marker expression was observed in most cells of the BHK monolayers, indicating the reconstitution of fluorescent recombinant sMVA expression vectors. The recombinant sMVA expression vectors were prepared by freeze/thaw technique, titrated on BHK cells, and used to infect BHK cell monolayers at low MOI. At 16-24 hpi, the infected BHK cell monolayers were evaluated by IF imaging to confirm the formation and reconstitution of sMVA expressing different fluorescent marker sequences. This analysis revealed the formation of viral foci that showed triple fluorescence expression in BHK cell monolayers infected with F1-GFP/F2-RFP/F3-BFP-derived sMVA; double fluorescent expression in BHK cell monolayers infected with F1-GFP/F2-RFP/F3, F1-GFP/F2/F3-BFP, or F1/F2-RFP/F3-BFP-derived sMVA; and single fluorescence expression in BHK cell monolayers infected with F1-GFP/F2/F3, F1/F2-RFP/F3, or F1/F2/F3-BFP-derived sMVA. These results in sum indicate that the three sMVA fragments can be manipulated in E. coli and used as circular DNA molecules to reconstitute single, double, or triple recombinant sMVA expression vectors, highlighting the potential of the three sMVA fragments to generate recombinant vaccine vectors.

### Example 5: Construction and Evaluation of an Example of an Infectious Disease Antigen Modified MVA

To demonstrate utility of the sMVA platform to generate a multi-antigenic vaccine vector for infectious disease, the construction of rsMVA expressing the five-member HCMV envelope pentamer complex (PC) composed of gH, gL, UL128, UL130, and UL131A is evaluated. Antigen expression cassettes composed of 2A-linked gH/gL and UL128/UL130/UL131A subunits are separately inserted into the IGR69/70 located in sMVA F2 and the Del3 site located sMVA F3 using En Passant mutagenesis in E. coli. The modified sMVA fragments of F2 and F3 are transfected together with the unmodified sMVA fragment of F1 into FPV-infected BHK cells to reconstitute sMVA expressing all five PC subunits (rsMVA-PC). As a control, rsMVA expressing only the gH/gL or UL128/130/131A subunits is generated as well as sMVA without any inserted antigen sequences. Expression of the antigen sequences is confirmed by Western Blot, and the formation of multi-protein complexes by the PC subunits expressed from rsMVA-PC is verified by flow cytometry (FC) using neutralizing antibodies (NAb) that target conformational epitopes formed by two or more subunits of the PC. To investigate the immunogenicity of the rsMVA-PC vector to stimulate NAb, Balb/c mice are immunized three times with rsMVA-PC or the control vectors by intraperitoneal route in four-week intervals. At various time points post immunization, HCMV-specific NAb responses are measured on MRC-5 fibroblasts (FB) and ARPE-19 epithelial cells (EC). This analysis can reveal potent stimulation of FB and EC-specific NAb responses in mice immunized with rsMVA-PC, while no or only minimal responses are measured in mice immunized with the control vectors. These results can demonstrate that the sMVA vaccine platform can be used to generate a multi-antigenic MVA vaccine vector that expresses all five PC subunits which assemble with each other to form NAb epitopes and stimulate potent NAb responses in mice.

### Example 6: Construction and Evaluation of 2019-nCOV Expressed from sMVA

An example for the utility of the sMVA platform to generate vaccines for the Coronavirus strain 2019-nCOV (Wuhan), which has recently emerged as a new, highly contagious and pathogenic Coronavirus is illustrated. Because the S (Spike) protein of Coronaviruses is involved in receptor recognition, as well as virus attachment and entry, and a dominant target of protective humoral and cellular immunity it represents a potential target for the development of Coronavirus (CoV) vaccines and therapeutics. cDNAs encoding the full-length S protein (˜1273 amino acids), the immunogenic S1 domain of the S protein of the 2019-nCOV strain, or the receptor binding domain (RBD) of the S1 domain are separately inserted into the MVA Del3 site of sMVA fragment F3. The resulting modified fragment of sMVA F3 is then co-transfected with the unmodified versions of sMVA fragment F1 and F2 into BHK cells to initiate the reconstitution of sMVA expressing the new coronavirus S protein (SMVA-2019-nCOV-S), the S1 domain (sMVA-2019-nCOV-S1), or the RBD domain (sMVA-2019-nCOV-RBD) using the procedure as illustrated in FIG. 5. The expression of the S protein, S1 domain, or the RBD domain from the sMVA recombinants is confirmed by Immunoblot and flow cytometry analysis. Convalescent serum from infected humans, mice or rabbits is used as a detection reagent for the immunoblots. In some cases, tags such as HA are covalently attached at the carboxyl terminus, and antibodies against them are used as detection reagents for either immunoblots or flow cytometry. Similarly, additional immunogenic proteins, e.g. N or M antigens from 2019-nCOV are inserted into unique gene insertion regions of sMVA to develop multi-antigen vaccines. For example, one or more of these 2019-nCOV antigens are inserted in one or more insertion sites such as Del3 site of sMVA fragment F3 and IGR69/70 of sMVA fragment F2.

To test the immunogenicity and protective efficacy of the sMVA constructs two animal models can be used: (1) Given the more severe symptoms observed in elderly individuals, 12 months old Balb/c mice are immunized two times by subcutaneous and intramuscular routes in four-week intervals with 1×10⁷-1×108 of sMVA plaque forming units (Pfu); and (2) Considering the potential involvement of human angiotensin-converting enzyme-2 (hACE-2) in nCOV entry into lung cells, transgenic mice expressing the human hACE2 receptor are used to test vaccine efficacy.

To test the immunogenicity and protective efficacy of the sMVA constructs, Balb/c mice transgenic for angiotensin converting enzyme 2 (ACE2) are immunized two times by subcutaneous and intramuscular routes in four-week intervals with 1×10⁷-1×108 of sMVA plaque forming units (Pfu). Neutralizing antibody responses and T cell responses specific for the 2019-nCOV-S protein and derivatives are measured as previously described at 1 week after each immunization. At 45 days post booster immunization, the mice are infected with 7×10⁴ 50% tissue culture infective doses (TICD50) of 2019-nCOV that is homologous to the vaccine antigens, and heterologous strains (SARS-COV and MERS-COV) and 4 days post challenge, the animals are sacrificed, and their lungs are harvested for measurement of viral loads and for histopathological analysis. Large virus loads, on average, >11,000 to >20,000 2019-nCOV genome equivalents/ng of total RNA, are found in both mock-immunized and nonrecombinant MVA-immunized control groups and in animals challenged with heterologous CoV strains. In sharp contrast, the lung tissue of protein (sMVA-nCOV-S) or the S1 domain (sMVA-nCOV-S1) subjects contains significantly lower levels of 2019-nCOV RNA (viral load), indicating efficient inhibition of 2019-nCOV replication by the vaccine-induced immune responses. All placebo control animals (MVA-GFP) succumbed 4 to 8 days postinfection, while MVA-S, MVA-N and MVA-N/S show no or minimal signs of disease, including minimal weight loss. MVA-N/S immunized mice are completely protected.

Histopathology is focused on lung damage that is substantially reduced in the 2019-nCOV subunit antigen immunized animals. The potential for antibody-enhancement of disease is also evaluated and found to be absent in these subunit vaccine models. NAb responses to S protein antigens and their S1 and RBD derivatives are assessed using a pseudotyping strategy for nCOV that is robust for recognition by antigen-specific NAb, but the use of pathogenic strains of CoV is avoided. The serum from animals immunized with S proteins and derivatives develops high titer and neutralizing antibodies against homologous 2019-nCOV strains and not heterologous CoV strains such as the etiologic agents for MERS and SARS. IC90 neutralizing titers are calculated for each mouse serum sample. Based on the success of the vaccine studies in transgenic ACE2 Balb/c mouse strains, progressively larger animal species are investigated, i.e. rabbits, ferrets, and rhesus macaques which have susceptibility to 2019-nCOV challenge and are known to respond to MVA-based vaccines.

### Example 7: Construction and Evaluation of an Example of a Cancer Antigen Modified sMVA

This example demonstrates insertion of a mouse p53 gene that has been previously used in studies with MVA backbones obtained from the ATCC or the NIAID46. The mouse p53 cDNA is inserted into the deletion (DEL3) locus using en passant such that no additional nucleotide additions or subtractions are made in the exact insertion site of the locus. The insertion is verified by sequence analysis to be accurately made and all pertinent regions are intact and 100% sequence verifiable. Subsequent to the modification of plasmid F3, all three plasmids are combined into a single aliquot and using the identical method as described in Example 2, a modified version of sMVA is generated, and live virus is obtained two days after the transfection/infection with FPV. The live virus is concentrated and expanded on BHK cells and stocks are made, and are sequence verified using the Sanger method to establish that the virus has the correct sequence of the p53 gene insertion in the DEL3 locus. Furthermore, virus stocks are made and are frozen and put into long-term storage, while at the same time, working stocks are prepared using the published methods to expand modified sMVA stocks to titers in ≥1×10⁹ pfu/mL. The verification of the expanded stock is conducted using a combination of qPCR, Sanger sequencing, infectious titer, and Western blot analysis. All four of these procedures are performed and the appropriate result is obtained to confirm that a nucleotide sequence has the appropriate size, the infectious titer is within the range of ≥1×10⁹ pfu/mL, and Western blot analysis confirms a protein band at approximately 53 Kd. One skilled in the art can apply this approach using human versions of the p53 gene as a vaccine to protect humans from cancer progression.

Murine (Mu) p53-sMVA

Female 6-8-week old BALB/C mice are obtained from the Jackson Laboratory (Bar Harbor, ME) and maintained in a specific pathogen-free environment. All studies are approved by the Research Animal Care Committee of City of Hope National Medical Center and performed under the American Association for the Accreditation of Laboratory Animal Care guidelines. Meth A sarcoma cells (Meth A) are a kind gift of Dr. LJ Old, Memorial Sloan-Kettering Cancer Center (New York, NY). Meth A is passaged as an ascitic tumor. Anti-CTLA-4 (9H10) is a gift of Dr. James Allison, MD Anderson Cancer Center (Houston, TX). Antibodies are produced using the CELLine device (BD Biosciences, Mountain View, CA). IgG antibodies are purified by absorption over protein G-sepharose (Amersham Biosciences, Uppsala, Sweden) followed by elution with 0.1 M glycine-HCL (pH 2.7). The product is dialyzed against phosphate-buffered normal saline and concentrated using a Sentry Plus centrifugal filter device (Millipore, Bedford, MA). Control Syrian hamster IgG is obtained from Jackson ImmunoResearch (West Grove, PA). The rsMVA titer is determined by immunostaining infected cultures using the VECTASTAIN Elite ABC Kit (Vector Laboratories, Burlingame, CA). Antigen-specific detection of the mouse p53 protein expressed from sMVA uses the anti-p53 antibody, pab122, followed by incubation with a peroxidase-labeled goat anti-mouse secondary antibody provided in the kit. A control sMVA expressing CMV-pp65 is also constructed using the same technique as is used in constructing murine sMVAp53.

### In Vivo Tumor Challenge Experiments

Six-week-old female BALB/C mice are injected by subcutaneous route in the left flank with 5×10⁵ METH A cells. On day three, the mice are treated with 5×10⁷ pfu of sMVAp53 by intraperitoneal injection. Negative control mice are injected with 5×10⁷ pfu of sMVApp65 or phosphate buffered saline. An additional positive control utilizes the identical murine MVAp53 described in the original report. The subcutaneous tumors are evaluated by IVIS imaging methods weekly using a luciferase technique or by calipers if no luciferase gene is inserted into the METH A sarcoma cells. An additional approach is to inject BALB/C mice of the same age as the sMVA infections in the left flank with 1×10⁶ METH A cells. This tumor is shown to produce a rapidly lethal tumor in the majority of mice despite CTLA-4 (9H10) antibody treatment. On day seven post tumor inoculation, mice are injected intraperitoneally with 5×10⁷ pfu of sMVAp53. Controls are the same as described above. Anti-CTLA-4 (9H10) antibody or control hamster antibody are injected intraperitoneally on day six, nine, and twelve post tumor injection at doses of 100, 50, and 50 micrograms, respectively.

### Example 8: Construction and In Vitro and In Vivo Characterization of sMVA

The three sMVA fragments designed as shown in FIG. 1, when co-transfected as circular DNA plasmids into helper virus infected cells, can resolve into linear minichromosomes, recombine with each other via the shared homologous sequences, and are ultimately packaged as full-length MVA genomes. All three sMVA fragments were cloned in E. coli as bacterial artificial chromosome (BAC) clones.

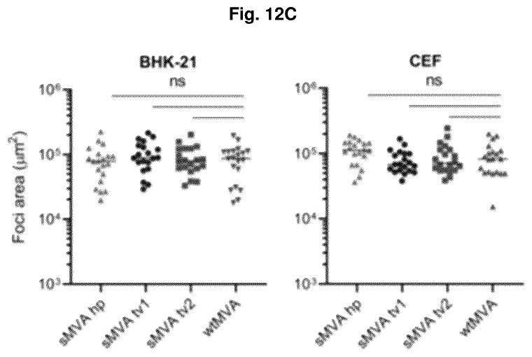

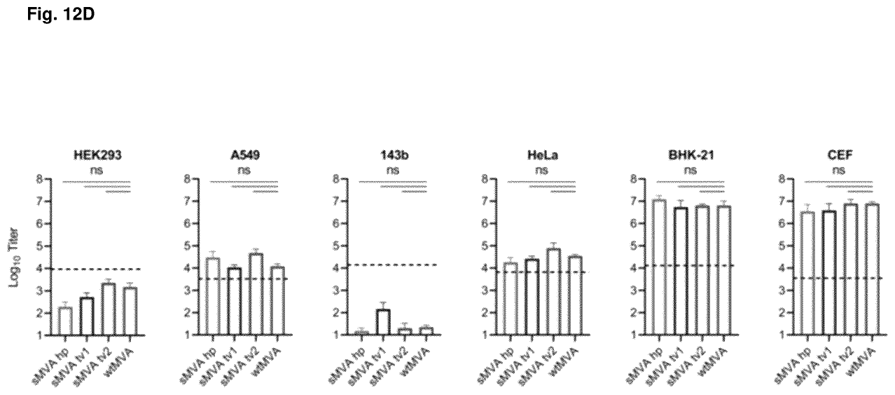

Using a previously employed procedure to rescue MVA from a BAC16,24,25, sMVA virus was reconstituted with Fowl pox (FPV) as a helper virus upon co-transfection of the three DNA plasmids into BHK cells (FIG. 2), which are non-permissive for FPV. Two different FPV strains (HP1.441 and TROVAC) were used to promote sMVA virus reconstitution (FIG. 12A). Ultra-purified sMVA virus was produced following virus propagation in CEF, which are commonly used for MVA vaccine production. The virus titers achieved with reconstituted sMVA virus were similar to virus titers achieved with wild-type MVA (wtMVA) (Table 1 below).

| Vector | Titer* |
|---|---|
| sMVA hp | 6.8 × 10⁹ PFU/ml |
| sMVA tv1 | 4.1 × 10⁹ PFU/ml |
| sMVA tv2 | 2.3 × 10⁹ PFU/ml |
| wtMVA | 4.1 × 10⁹ PFU/ml |

*Stocks were produced on CEF following infection (MOI 0.02) of 30 × 15 cm dishes

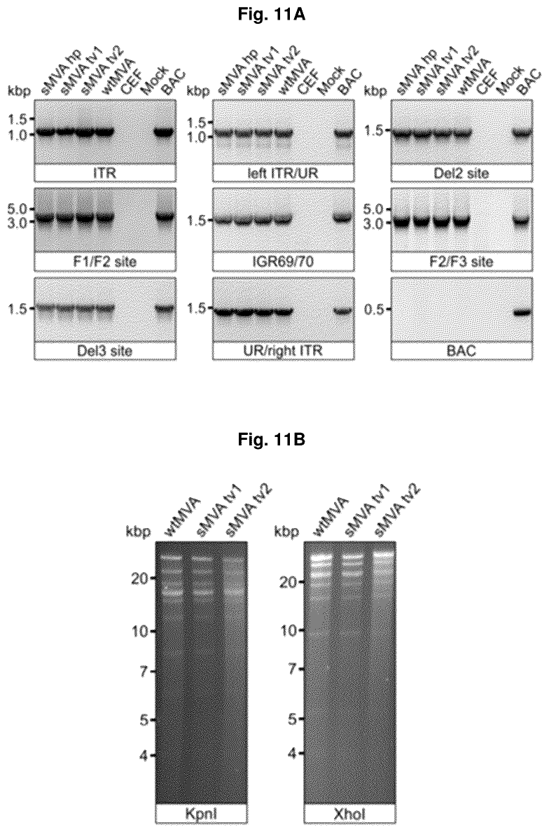

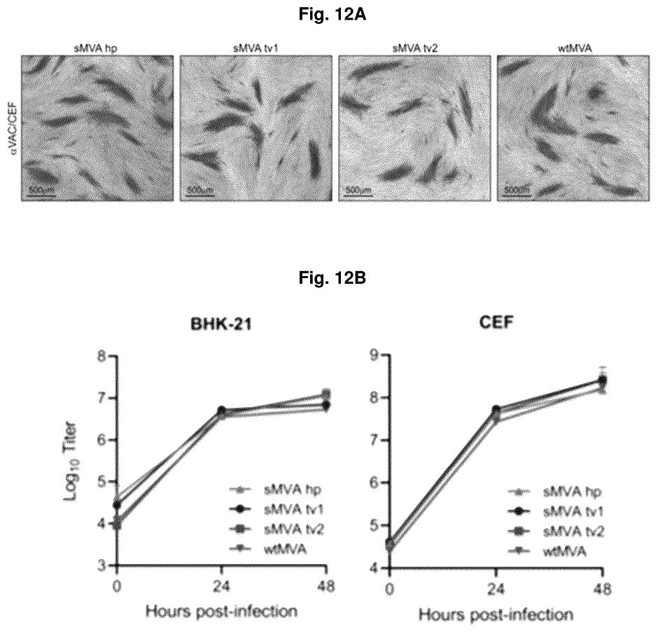

To characterize the viral DNA of sMVA, DNA extracts from sMVA and wtMVA-infected CEF were compared for several MVA genome positions by PCR. Similar PCR results were obtained with sMVA and wtMVA for all evaluated genome positions (FIG. 11A), including the F1/F2 and F2/F3 recombination sites, indicating efficient recombination of the three sMVA fragments. Additional PCR analysis indicated the absence of any BAC vector sequences in sMVA viral DNA (FIG. 11A), suggesting spontaneous and efficient removal of the bacterial vector elements upon sMVA virus reconstitution. Comparison of viral DNA from ultra-purified sMVA and wtMVA virus by restriction enzyme digestion revealed similar genome pattern between sMVA and wtMVA (FIG. 11B). Sequencing analysis of the sMVA viral DNA confirmed the MVA genome sequence at several positions, including the F1/F2 and F2/F3 recombination sites. Furthermore, whole genome sequencing of one of the sMVA virus isolates reconstituted with FPV TROVAC confirmed the assembly of the reference MVA genome sequence and absence of vector-specific sequences in viral DNA originating from the reconstituted sMVA virus.

To characterize the replication properties of sMVA, growth kinetics of sMVA and wtMVA were compared on BHK and CEF cells, two cell types known to support productive MVA replication⁴. This analysis revealed similar growth kinetics of sMVA and wtMVA on both BHK and CEF cells (FIG. 12B). In addition, similar areas of viral foci were determined in BHK and CEF cell monolayers infected with sMVA or wtMVA (FIG. 12C), suggesting similar capacity of sMVA and wtMVA to spread in MVA permissive cells. Compared to the productive replication of sMVA and wtMVA in BHK and CEF cells⁴, only limited virus production was observed with sMVA or wtMVA following infection of various human cell lines (FIG. 12D). These results are consistent with the severely restricted replication properties of MVA and show that the sMVA virus can efficiently propagate in BHK and CEF cells, while it is unable to efficiently propagate in human cells.

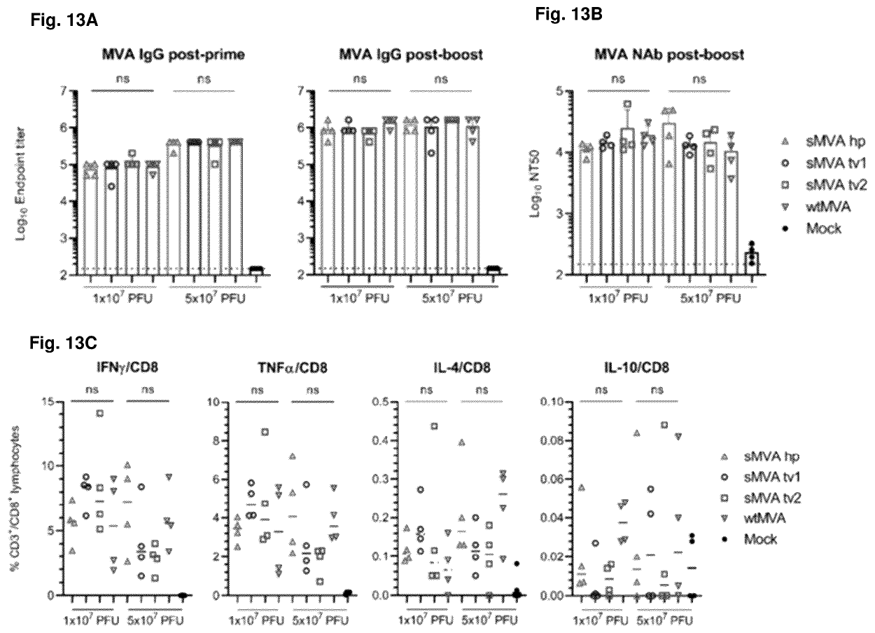

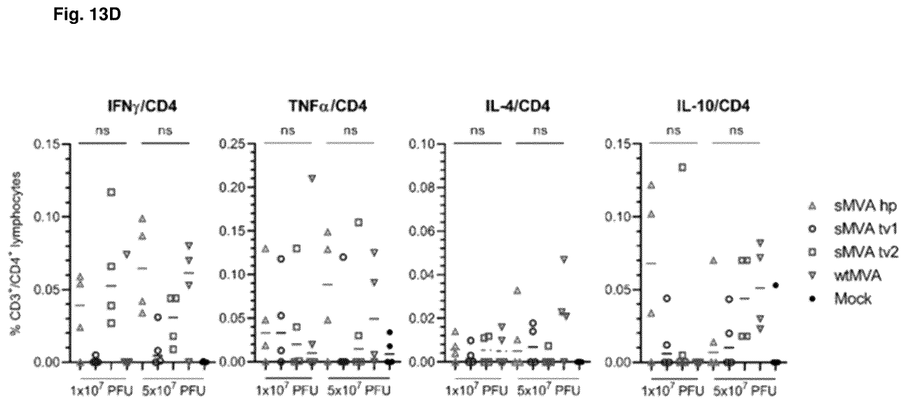

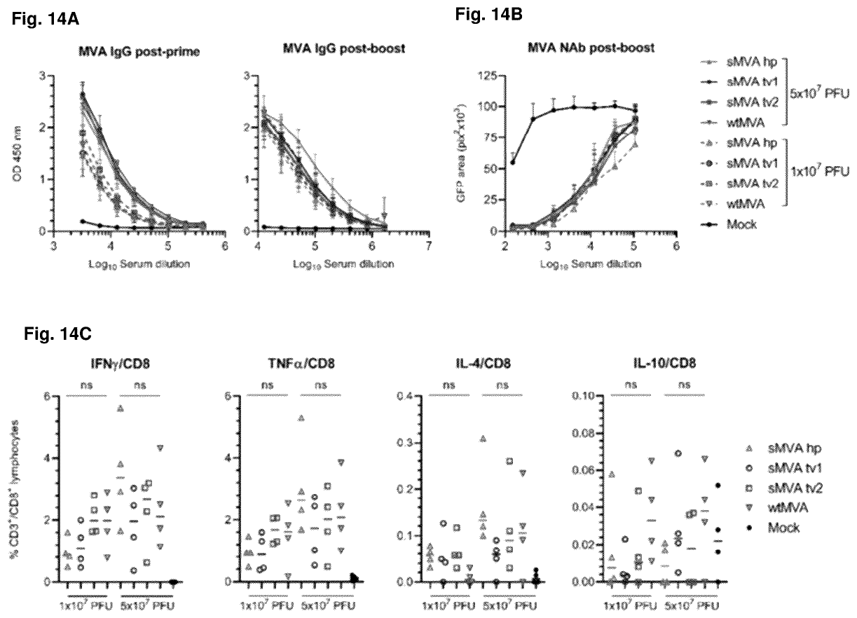

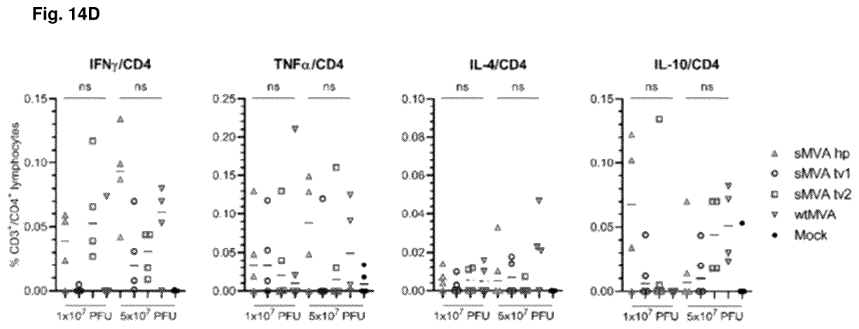

To characterize sMVA in vivo, the immunogenicity of sMVA and wtMVA was compared in C57BL/6 mice following two immunizations at high or low dose. MVA-specific binding antibodies stimulated by sMVA and wtMVA after the first and second immunization were comparable (FIGS. 13A, 14A). While the antibody levels in the high dose vaccine groups exceeded those of the low dose vaccine groups after the first immunization, similar antibody levels in the high and low dose vaccine groups were observed after the second immunization. In addition, no significant differences were detected in the levels of MVA-specific NAb responses induced by sMVA and wtMVA after the second immunization (FIGS. 13B, 14B). MVA-specific T cell responses determined after the booster immunization by ex vivo antigen stimulation using immunodominant peptides⁴⁷ revealed similar MVA-specific T cell levels in mice receiving sMVA or wtMVA (FIGS. 13C-13D and 14C-14D). These results indicate that the sMVA virus has a similar capacity to wtMVA in inducing MVA-specific humoral and cellular immunity in mice.

### REFERENCES

The references, patents and published patent applications listed below, and all references cited in the specification above are hereby incorporated by reference in their entirety, as if fully set forth herein.
1 Volz, A. & Sutter, G. Modified Vaccinia Virus Ankara: History, Value in Basic Research, and Current Perspectives for Vaccine Development. Adv. Virus Res. 97, 187-243, doi: 10.1016/bs.aivir.2016.07.001 (2017).
2 Meisinger-Henschel, C. et al. Genomic sequence of chorioallantois vaccinia virus Ankara, the ancestor of modified vaccinia virus Ankara. The Journal of general virology 88, 3249-3259, doi: 10.1099/vir.0.83156-0 (2007).
3 Meyer, H., Sutter, G. & Mayr, A. Mapping of deletions in the genome of the highly attenuated vaccinia virus MVA and their influence on virulence. The Journal of general virology 72 (Pt 5), 1031-1038 (1991).
4 Carroll, M. W. & Moss, B. Host range and cytopathogenicity of the highly attenuated MVA strain of vaccinia virus: propagation and generation of recombinant viruses in a nonhuman mammalian cell line. Virology 238, 198-211, doi: 10.1006/viro. 1997.8845 (1997).
5 Sutter, G. & Moss, B. Nonreplicating vaccinia vector efficiently expresses recombinant genes. Proc. Natl. Acad. Sci. U.S.A 89, 10847-10851 (1992).
6 Earl, P. L. et al. Immunogenicity of a highly attenuated MVA smallpox vaccine and protection against monkeypox. Nature 428, 182-185, doi: 10.1038/nature02331 (2004).
7 Gilbert, S. C. Clinical development of Modified Vaccinia virus Ankara vaccines. Vaccine 31, 4241-4246, doi: 10.1016/j.vaccine.2013.03.020 (2013).
8 Mayr, A., Stickl, H., Muller, H. K., Danner, K. & Singer, H. [The smallpox vaccination strain MVA: marker, genetic structure, experience gained with the parenteral vaccination and behavior in organisms with a debilitated defence mechanism (author's transl)]. Zentralbl Bakteriol B 167, 375-390 (1978).
9 Wilck, M. B. et al. Safety and immunogenicity of modified vaccinia Ankara (ACAM3000): effect of dose and route of administration. J. Infect. Dis. 201, 1361-1370, doi: 10.1086/651561 (2010).
10 Vollmar, J. et al. Safety and immunogenicity of IMVAMUNE, a promising candidate as a third generation smallpox vaccine. Vaccine 24, 2065-2070, doi: 10.1016/j.vaccine.2005.11.022 (2006).
11 Greenberg, R. N. et al. A Multicenter, Open-Label, Controlled Phase II Study to Evaluate Safety and Immunogenicity of MVA Smallpox Vaccine (IMVAMUNE) in 18-40 Year Old Subjects with Diagnosed Atopic Dermatitis. PloS one 10, e0138348, doi: 10.1371/journal.pone.0138348 (2015).
12 Draper, S. J., Cottingham, M. G. & Gilbert, S. C. Utilizing poxviral vectored vaccines for antibody induction-progress and prospects. Vaccine 31, 4223-4230, doi: 10.1016/j.vaccine.2013.05.091 (2013).
13 Cottingham, M. G. & Carroll, M. W. Recombinant MVA vaccines: dispelling the myths. Vaccine 31, 4247-4251, doi: 10.1016/j.vaccine.2013.03.021 (2013).
14 Chung, V. et al. Evaluation of safety and efficacy of p53MVA vaccine combined with pembrolizumab in patients with advanced solid cancers. Clinical & translational oncology: official publication of the Federation of Spanish Oncology Societies and of the National Cancer Institute of Mexico 21, 363-372, doi: 10.1007/s12094-018-1932-2 (2019).
15 Ishizaki, H. et al. Modified vaccinia Ankara expressing survivin combined with gemcitabine generates specific antitumor effects in a murine pancreatic carcinoma model. Cancer Immunol. Immunother. 60, 99-109, doi: 10.1007/s00262-010-0923-0 (2011).
16 Wussow, F. et al. Human cytomegalovirus vaccine based on the envelope gH/gL pentamer complex. PLOS Pathog 10, e1004524, doi: 10.1371/journal.ppat.1004524 (2014).
17 La Rosa, C. et al. MVA vaccine encoding CMV antigens safely induces durable expansion of CMV-specific T cells in healthy adults. Blood 129, 114-125, doi: 10.1182/blood-2016-07-729756 (2017).
18 Chiuppesi, F. et al. Multiantigenic Modified Vaccinia Virus Ankara Vaccine Vectors To Elicit Potent Humoral and Cellular Immune Reponses against Human Cytomegalovirus in Mice. Journal of virology 92, doi: 10.1128/JVI.01012-18 (2018).
19 Diamond, D. J. et al. A fifty-year odyssey: prospects for a cytomegalovirus vaccine in transplant and congenital infection. Expert review of vaccines 17, 889-911, doi: 10.1080/14760584.2018.1526085 (2018).
20 McFadden, G. Poxvirus tropism. Nature reviews. Microbiology 3, 201-213, doi: 10.1038/nrmicro1099 (2005).
21 Wyatt, L. S., Earl, P. L. & Moss, B. Generation of Recombinant Vaccinia Viruses. Current protocols in protein science 89, 5 13 11-15 13 18, doi: 10.1002/cpps.33 (2017).
22 Wang, Z. et al. Modified H5 promoter improves stability of insert genes while maintaining immunogenicity during extended passage of genetically engineered MVA vaccines. Vaccine 28, 1547-1557, doi: 10.1016/j.vaccine.2009.11.056 (2010).
23 Cottingham, M. G. et al. Recombination-mediated genetic engineering of a bacterial artificial chromosome clone of modified vaccinia virus Ankara (MVA). PloS one 3, e1638, doi: 10.1371/journal.pone.0001638 (2008).
24 Wussow, F. et al. Exploiting 2A peptides to elicit potent neutralizing antibodies by a multi-subunit herpesvirus glycoprotein complex. J. Virol. Methods 251, 30-37, doi: 10.1016/j.jviromet.2017.10.006 (2018).
25 Wussow, F. et al. A vaccine based on the rhesus cytomegalovirus UL128 complex induces broadly neutralizing antibodies in rhesus macaques. Journal of virology 87, 1322-1332, doi: 10.1128/JVI.01669-12 (2013).
26 Antoine, G., Scheiflinger, F., Dorner, F. & Falkner, F. G. The complete genomic sequence of the modified vaccinia Ankara strain: comparison with other orthopoxviruses. Virology 244, 365-396, doi: 10.1006/viro. 1998.9123 (1998).
27 Moss, B. Poxvirus DNA replication. Cold Spring Harbor perspectives in biology 5, doi: 10.1101/cshperspect.a010199 (2013).
28 Geshelin, P. & Berns, K. I. Characterization and localization of the naturally occurring cross-links in vaccinia virus DNA. Journal of molecular biology 88, 785-796, doi: 10.1016/0022-2836 (74) 90399-4 (1974).
29 Baroudy, B. M., Venkatesan, S. & Moss, B. Incompletely base-paired flip-flop terminal loops link the two DNA strands of the vaccinia virus genome into one uninterrupted polynucleotide chain. Cell 28, 315-324, doi: 10.1016/0092-8674 (82) 90349-x (1982).
30 Baroudy, B. M., Venkatesan, S. & Moss, B. Structure and replication of vaccinia virus telomeres. Cold Spring Harbor symposia on quantitative biology 47 Pt 2, 723-729, doi: 10.1101/sqb.1983.047.01.083 (1983).
31 Merchlinsky, M., Garon, C. F. & Moss, B. Molecular cloning and sequence of the concatemer junction from vaccinia virus replicative DNA. Viral nuclease cleavage sites in cruciform structures. Journal of molecular biology 199, 399-413, doi: 10.1016/0022-2836 (88) 90613-4 (1988).
32 Moyer, R. W. & Graves, R. L. The mechanism of cytoplasmic orthopoxvirus DNA replication. Cell 27, 391-401, doi: 10.1016/0092-8674 (81) 90422-0 (1981).
33 DeLange, A. M. & McFadden, G. Efficient resolution of replicated poxvirus telomeres to native hairpin structures requires two inverted symmetrical copies of a core target DNA sequence. Journal of virology 61, 1957-1963 (1987).
34 Merchlinsky, M. Mutational analysis of the resolution sequence of vaccinia virus DNA: essential sequence consists of two separate AT-rich regions highly conserved among poxviruses. Journal of virology 64, 5029-5035 (1990).
35 Garcia, A. D. & Moss, B. Repression of vaccinia virus Holliday junction resolvase inhibits processing of viral DNA into unit-length genomes. Journal of virology 75, 6460-6471, doi: 10.1128/JVI.75.14.6460-6471.2001 (2001).
36 Merchlinsky, M. & Moss, B. Resolution of vaccinia virus DNA concatemer junctions requires late-gene expression. Journal of virology 63, 1595-1603 (1989).
37 DeLange, A. M., Reddy, M., Scraba, D., Upton, C. & McFadden, G. Replication and resolution of cloned poxvirus telomeres in vivo generates linear minichromosomes with intact viral hairpin termini. Journal of virology 59, 249-259 (1986).
38 DeLange, A. M. & McFadden, G. Sequence-nonspecific replication of transfected plasmid DNA in poxvirus-infected cells. Proceedings of the National Academy of Sciences of the United States of America 83, 614-618, doi: 10.1073/pnas.83.3.614 (1986).
39 Tischer, B. K., von Einem, J., Kaufer, B. & Osterrieder, N. Two-step red-mediated recombination for versatile high-efficiency markerless DNA manipulation in Escherichia coli. BioTechniques 40, 191-197 (2006).
40 Tischer, B. K., Smith, G. A. & Osterrieder, N. En passant mutagenesis: a two step markerless red recombination system. Methods in molecular biology 634, 421-430, doi: 10.1007/978-1-60761-652-8_30 (2010).
41 Birnboim, H. C. & Doly, J. A rapid alkaline extraction procedure for screening recombinant plasmid DNA. Nucleic acids research 7, 1513-1523, doi: 10.1093/nar/7.6.1513 (1979).
42 Schmelz, M. et al. Assembly of vaccinia virus: the second wrapping cisterna is derived from the trans Golgi network. Journal of virology 68, 130-147 (1994).
43 Gilbert, S. C. Clinical development of Modified Vaccinia virus Ankara vaccines. Vaccine 31, 4241-4246 (2013).
44 Scheiflinger, F., Dorner, F. & Falkner, F. G. Construction of chimeric vaccinia viruses by molecular cloning and packaging. Proc. Natl. Acad. Sci. U.S.A 89, 9977-9981, doi: 10.1073/pnas.89.21.9977 (1992).
45 Wyatt, L. S. et al. Elucidating and minimizing the loss by recombinant vaccinia virus of human immunodeficiency virus gene expression resulting from spontaneous mutations and positive selection. Journal of virology 83, 7176-7184, doi: 10.1128/JVI.00687-09 (2009).
46 Espenschied J, Lamont J, Longmate J, Pendas S, Wang Z, Diamond DJ, and Ellenhorn JD. CTLA-4 blockade enhances the therapeutic effect of an attenuated poxvirus vaccine targeting p53 in an established murine tumor model. J Immunol 170 (6): 3401-3407, 2003, PMID: 12626601.
47. Taylor, J., Weinberg, R., Kawaoka, Y., Webster, R. G. & Paoletti, E. Protective immunity against avian influenza induced by a fowlpox virus recombinant. Vaccine 6, 504-508 (1988).
48. Mayr, A. & Malicki, K. [Attenuation of virulent fowl pox virus in tissue culture and characteristics of the attenuated virus]. Zentralbl. Veterinarmed. B 13, 1-13 (1966).

1. A vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-COV-2 Nucleocapsid (N) protein, wherein the first DNA sequence is inserted in a first insertion site of the full-length sMVA genome; and
(iii) a second DNA sequence encoding a SARS-COV-2 Spike(S) protein, wherein the second DNA sequence is inserted in a second insertion site of the full-length sMVA genome.

2. The vaccine composition of claim 1, wherein the full-length sMVA genome comprises a nucleotide sequence identical or substantially identical to MVA strain Antoine (Accession No. U94848).

3. The vaccine composition of claim 1, wherein the full length sMVA genome comprises a nucleotide sequence identical or substantially identical to MVA strain Acambis (Accession No. AY603355).

4. The vaccine composition of claim 1, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

5. The vaccine composition of claim 4, wherein the first insertion site is Del2.

6. The vaccine composition of claim 1, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

7. The vaccine composition of claim 6, wherein the second insertion site is Del3.

8. The vaccine composition of claim 1, wherein the rsMVA is reconstituted from homologous recombination of three DNA fragments, F1, F2, and F3, wherein:
F1 comprises a first partial sequence of the full-length sMVA genome and the first DNA sequence encoding the SARS-COV-2 N protein;
F2 comprises a second partial sequence of the full-length sMVA genome; and
F3 comprises a third partial sequence of the full-length sMVA genome and the second DNA sequence encoding SARS-COV-2 S protein;
and wherein an MVA terminal hairpin loop (HL) sequence flanked by MVA concatemeric resolution (CR) sequences (CR/HL/CR) is added to both ends of each of F1, F2, and F3.

9. The vaccine composition of claim 8, wherein the HL sequence is selected from the group consisting of SEQ ID NO: 3, SEQ ID NO: 4, SEQ ID NO: 7, and SEQ ID NO: 8.

10. The vaccine composition of claim 8, wherein the CR sequence comprises SEQ ID NO: 9 or SEQ ID NO: 10.

11. A method of inducing an immune response to a SARS-COV-2 virus in a human subject in need thereof comprising administering to the subject a vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-COV-2 Nucleocapsid (N) protein, wherein the first DNA sequence is inserted in a first insertion site of the full-length sMVA genome; and
(iii) a second DNA sequence encoding a SARS-COV-2 Spike(S) protein, wherein the second DNA sequence is inserted in a second insertion site of the full-length sMVA genome.

12. The method of claim 11, wherein the full-length sMVA genome comprises a nucleotide sequence identical or substantially identical to MVA strain Antoine (Accession No. U94848).

13. The method of claim 11, wherein the full length sMVA genome comprises a nucleotide sequence identical or substantially identical to MVA strain Acambis (Accession No. AY603355).

14. The method of claim 11, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

15. The method of claim 14, wherein the first insertion site is Del2.

16. The method of claim 11, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

17. The method of claim 16, wherein the second insertion site is Del3.

18. The method of claim 11, wherein the rsMVA is reconstituted from homologous recombination of three DNA fragments, F1, F2, and F3, wherein:
F1 comprises a first partial sequence of the full-length sMVA genome and the first DNA sequence encoding the SARS-COV-2 N protein;
F2 comprises a second partial sequence of the full-length sMVA genome; and
F3 comprises a third partial sequence of the full-length sMVA genome and the second DNA sequence encoding SARS-COV-2 S protein;
and wherein an MVA terminal hairpin loop (HL) sequence flanked by MVA concatemeric resolution (CR) sequences (CR/HL/CR) is added to both ends of each of F1, F2, and F3.

19. The method of claim 18, wherein the HL sequence is selected from the group consisting of SEQ ID NO: 3, SEQ ID NO: 4, SEQ ID NO: 7, and SEQ ID NO: 8.

20. The method of claim 18, wherein the CR sequence comprises SEQ ID NO: 9 or SEQ ID NO: 10.

21. A method of preventing a SARS-COV-2 virus infection in a human subject in need thereof comprising administering to the subject a vaccine composition comprising a reconstituted recombinant synthetic modified vaccinia Ankara (rsMVA) virus, the rsMVA comprising:
(i) a full-length synthetic MVA (sMVA) genome;
(ii) a first DNA sequence encoding a SARS-COV-2 Nucleocapsid (N) protein, wherein the first DNA sequence is inserted in a first insertion site of the full-length sMVA genome; and
(iii) a second DNA sequence encoding a SARS-COV-2 Spike(S) protein, wherein the second DNA sequence is inserted in a second insertion site of the full-length sMVA genome.

22. The method of claim 21, wherein the full-length sMVA genome comprises a nucleotide sequence identical or substantially identical to MVA strain Antoine (Accession No. U94848).

23. The method of claim 21, wherein the full length sMVA genome comprises a nucleotide sequence identical or substantially identical to MVA strain Acambis (Accession No. AY603355).

24. The method of claim 21, wherein the first insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site, wherein the open reading frame numbers are based on MVA strain Antoine.

25. The method of claim 24, wherein the first insertion site is Del2.

26. The method of claim 21, wherein the second insertion site is selected from the group consisting of Del2, Del3, intergenic region (IGR) between open reading frame (ORF) 44L and 45L (IGR 44/45), intergenic region (IGR) between open reading frame (ORF) 64L and 65L (IGR 64/65), intergenic region (IGR) between open reading frame (ORF) 69R and 70L (IGR69/70), and Thymidine Kinase (TK) gene insertion site.

27. The method of claim 26, wherein the second insertion site is Del3.

28. The method of claim 21, wherein the rsMVA is reconstituted from homologous recombination of three DNA fragments, F1, F2, and F3, wherein:
F1 comprises a first partial sequence of the full-length sMVA genome and the first DNA sequence encoding the SARS-COV-2 N protein;
F2 comprises a second partial sequence of the full-length sMVA genome; and
F3 comprises a third partial sequence of the full-length sMVA genome and the second DNA sequence encoding SARS-COV-2 S protein;
and wherein an MVA terminal hairpin loop (HL) sequence flanked by MVA concatemeric resolution (CR) sequences (CR/HL/CR) is added to both ends of each of F1, F2, and F3.

29. The method of claim 28, wherein the HL sequence is selected from the group consisting of SEQ ID NO: 3, SEQ ID NO: 4, SEQ ID NO: 7, and SEQ ID NO: 8.

30. The method of claim 28, wherein the CR sequence comprises SEQ ID NO: 9 or SEQ ID NO: 10.
